(19) United States
(12) Patent Application Publication
(10) Pub. No.: US 2015/0018566 A1
(43) Pub. Date: Jan. 15, 2015

(54) TUBULIN BINDING AGENTS

(71) Applicant: The Provost, Fellows, Foundation Scholars, & the other Members of Board, of the College of the Holy, Dublin (IE)

(72) Inventors: John Jarlath Walsh, Dublin (IE); Richard Shah, Dublin (IE); Emmet Martin McCormack, Nesttun (NO); Gillian Joy Hudson, Dublin (IE); Martina White, Dublin (IE); Gary Daniel Stack, Co. Kerry (IE); Brian William Moran, Dublin (IE); Adrian Coogan, Co. Monaghan (IE); Elaine Carmel Breen, Co. Carlow (IE)

(21) Appl. No.: 14/240,985

(22) PCT Filed: Aug. 27, 2012
(86) PCT No.: PCT/EP2012/066627

§ 371 Date: Sep. 19, 2014

(30) Foreign Application Priority Data
Aug. 25, 2011 (EP) 11178918.6
Aug. 25, 2011 (EP) 11178919.4

(51) Int. Cl. C07C 43/23; C07D 313/08; C07D 311/16; C07C 237/22; C07C 259/06; C07C 225/22; C07C 251/44; C07C 49/755; C07F 9/655

(52) U.S. Cl. CPC C07C 43/23; C07C 49/755; C07D 313/08; C07D 311/16; C07F 9/65522; C07F 9/65527; C07C 259/06; C07C 225/22; C07C 251/44; C07C 237/22; C07C 2102/12
USPC 549/220; 568/327; 568/328; 549/355; 549/270; 549/289; 562/623; 564/308; 564/265; 568/633; 564/157

(57) ABSTRACT

The invention provides combretastatin A-4 like compounds that are modified to have enhanced tubulin binding activity and in some embodiments the ability to promote accumulation in the vasculature undergoing angiogenesis (homing activity). The compounds are based on the combretastatin A-4 skeletal structure having a tubulin-binding pharmacophore comprising two fused rings (A and B rings) in which the B ring is substituted with (a) an aromatic ring structure (C ring) and (b) a second substituent/functional group that comes off the B ring. The aromatic ring structure is typically a six membered ring phenolic or aniline structure, or may also be a fused ring structure such as a substituted or unsubstituted naphthalene. The second substituent on the B ring may for example be a substituent which has been found to provide enhanced tubulin binding activity (for example a carbonyl group), or may be a substituent that facilitates functionalisation of the B ring (for example an hydroxyl or amine group), or it may be a binding agent for a target that is preferentially expressed on vasculature undergoing angiogenesis, and not expressed on quiescent vasculature.

### TECHNICAL FIELD

The invention relates to compounds that function as tubulin binding agents capable of inhibiting tubulin assembly and tumour cell proliferation.

### BACKGROUND TO THE INVENTION

Cancer is a global problem and despite many promising leads, the ideal drug for the treatment of the ‘big five’, namely breast cancer, prostate cancer, non-small cell lung cancer (NSCLC), colorectal cancer and pancreatic cancer, still eludes the scientific community. The recent discovery of combretastatin A-4, a tubulin-binding compound that induces apoptosis in proliferating endothelial cells and causes tumour vascular shutdown has focused attention onto the re-direction of tubulin inhibitors to target tumour angiogenesis/vasculature rather than the tumour itself, on the basis that a solid tumour cannot survive or develop without a viable blood supply. Despite the relative success of combretastatin A-4, assessed in eighteen Phase I/II clinical trials in oncology and ophthalmology, its value is still compromised by a lack of specificity of the compound for the target tissue and cases of hypertension, tumor pain and intermittent cardiovascular toxicity.

Combretastatin A-4 analogs are described in WO2006/138427, including compounds having three methoxy substituents at the R4 to R6 positions of the A-ring and a B-ring that is substituted with a C-ring structure (see formula VII). However, the compounds of this document are limited in terms of functionalisation of the B-ring.

It is an object of the invention to overcome at least one of the above-referenced problems.

### STATEMENTS OF INVENTION

The invention provides combretastatin A-4 like compounds that are modified to have enhanced tubulin binding activity and in some embodiments the ability to promote accumulation in the vasculature undergoing angiogenesis (homing activity). The compounds are based on the combretastatin A-4 skeletal structure having a tubulin-binding pharmacophore comprising two fused rings (A and B rings) in which the B ring is substituted with (a) an aromatic ring structure (C ring) and (b) a second substituent/functional group that comes off the B ring. The aromatic ring structure is typically a six membered ring phenolic or aniline structure, or may also be a fused ring structure such as a substituted or unsubstituted naphthalene. The second substituent on the B ring may for example be a substituent which has been found to provide enhanced tubulin binding activity (for example a carbonyl group), or may be a substituent that facilitates functionalisation of the B ring (for example an hydroxyl or amine group), or it may be a binding agent for a target that is preferentially expressed on vasculature undergoing angiogenesis, and not expressed on quiescent vasculature. Examples of such targets are the enzymes aminopeptidase A (APA, EC 3.4.11.7) or aminopeptidase N (APN/CD13, 3.4.11.2). The compounds of the invention are additionally characterised by having three lower alkoxy groups on the A ring, typically at positions R₄ to R₆ in Formula I below. The Applicant has surprisingly discovered that substitution of the A ring with lower alkoxy groups at these positions provides tubulin binding agents with enhanced tubulin binding activity. The Applicant has additionally discovered that the presence of a carbonyl substituent on the B-ring confers enhanced tubulin binding activity on the compound. Further, the Applicant has discovered that the provision of a C-ring having a lower alkoxy substituent in the para position provides for enhanced tubulin binding activity.

The binding agents for a target that is preferentially expressed on vasculature undergoing angiogenesis, and not expressed on quiescent vasculature, are ligands for the target, for example, an inhibitor of the target, a substrate for the target, or antagonist of the target. When the binding agent is an inhibitor of the target, the compounds of the invention will have the additional advantage of having dual activity of tubulin binding and anti-angiogenesis, as APA and APN are required for angiogenesis. Such compounds are hereafter referred to as “dual activity compounds”.

Moreover, the Applicant has discovered that when a bulky side chain is attached as a substituent to the C ring of a tubulin binding compound, the tubulin binding activity of the resultant compound is abrogated until such time as the substituent is removed. This has enabled the generation of a class of pro-drug tubulin binding compounds, that have a bulky substituent engineered as a substituent off the C-ring (for example, an APA or APN substrate engineered into the compounds as a substituent of the C ring), the tubulin binding activity of which compounds are initially inactive until the APA or APN substrate is cleaved in-vivo due to the action of an APA or APN enzyme, respectively, whereupon the compound is activated. These compounds are hereafter referred to as “pro-drug compounds”. As the target enzymes are only expressed in vasculature undergoing angiogenesis, and not on quiescent vasculature, this results in the accumulation of the pro-drug compounds at the site of vasculature undergoing angiogenesis, in which the compounds initially have no tubulin binding activity (pro-drug form) but are activated at the target site by release of the APA or APN substrate.

Accordingly, the invention relates to a compound of general formula I:

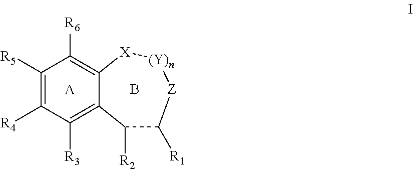

or a pharmaceutically acceptable salt thereof, wherein a dashed line indicates a single or double bond, and wherein:
   n=0, 1 or 2;
   X, Y and Z is each, independently, selected from the group consisting of a heteroatom (such as O or N), CH, CH₂, S═O, C═O, C═S, C(R), C(H)R, C(R)₂, N(R), C═NR, C═C(R)₂, C(L)W, C(H)LW, C(LW)₂, N(LW), C═N(LW), C═C(LW)₂;
   L is absent or any linker typically selected from O, NH, O-alkyl, CH₂O, CH₂NH, and CH₂NHCOCH₂, CO, COO;
   W is a binding agent for a target that is preferentially expressed on vasculature undergoing angiogenesis, and not expressed on quiescent vasculature, or an anti-angiogenic agent;
   R, R₁, R₂, and R₃ are each, independently, any substituent, typically selected from the groups consisting of H, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkoxycarbonylaminohydroxyl, aminocarbonyl, alkylthiocarbonyl, alkoxy, phosphate, phosphonato, phosphinato, cyano, amino including alkyl amino, dialkylamino, arylamino, diarylamino, and alkylarylamino, acylamino including alkylcarbonylamino, arylcarbonylamino, carbamoyl, and ureido, amidino, imino, sulfhydryl, alkylthio, arylthio, thiocarboxylate, sulphate, sulfonato, sulfamoyl, sulfonamido, nitro including nitrile, trifluoromethyl, isocyanate, isothiocyanate, azido, heterocycle, or a substituted or unsubstituted aromatic or heterocyclic ring structure in which the substituents (if included) are each, independently, selected from the groups consisting of W, (L)W, H, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkoxycarbonylaminohydroxyl aminocarbonyl, alkylthiocarbonyl, alkoxy, phosphate, phosphonato, phosphinato, cyano, amino including alkyl amino, dialkylamino, arylamino, diarylamino, and alkylarylamino, acylamino including alkylcarbonylamino, arylcarbonylamino, carbamoyl, and ureido, amidino, imino, sulfhydryl, alkylthio, arylthio, thiocarboxylate, sulphate, sulfonato, sulfamoyl, sulfonamido, nitro including nitrile, trifluoromethyl, isocyanate, isothiocyanate, azido, heterocycle,
   at least one of R₁ and R₂ is a substituted or unsubstituted aromatic or heterocyclic ring structure in which the substituents (if included) are each, independently, selected from the groups consisting of W, (L)W, H, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkoxycarbonylaminohydroxyl aminocarbonyl, alkylthiocarbonyl, alkoxy, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl, and ureido), amidino, imino, sulfhydryl, alkylthio, arylthio, thiocarboxylate, sulphate, sulfonato, sulfamoyl, sulfonamido, nitro (including nitrile), trifluoromethyl, isocyanate, isothiocyanate, azido, and a heterocycle; and
   R₄, R₅, and R₆ are each, independently, lower alkoxy substituents,
with the proviso that at least one of X, Y or Z is not a heteroatom, CH, or CH₂.

Compounds according to the invention have been shown to have effective tubulin binding activity, in some cases comparable to or better than Combretastatin. This activity is at least partly due to the arrangement of the lower alkoxy groups at the R₄ to R₆ positions on the A-ring. The compounds of the invention also have a second functional group coming off the B-ring (the first being the C-ring) that allows for functional group diversification, and can be selected to enhance the tubulin binding activity of the compound, or to provide anti-angiogenic activity mediated by means of an APA or APN inhibitor.

Typically, at least one of X, Y and Z is selected from the group consisting of a heteroatom (such as O or N), S═O, C═O, C═S, C(R), C(H)R, C(R)₂, N(R), C═NR, C═C(R)₂, C(L)W, C(H)LW, C(LW)₂, N(LW), C═N(LW), C═C(LW)₂.

Preferably, at least one of X, Y and Z, and more preferably Y or Z, and ideally Z, is a carbonyl group. The Applicant has surprisingly discovered that functionalisation of the B ring with a carbonyl group enhances the tubulin binding activity of the compound. This enhancement has been found to be further increased when the C-ring is substituted with a lower alkoxy group, preferably at the para position relative to the point of attachment of C-ring onto the B-ring.

Preferably, the other of X, Y and Z are each, independently, selected from CH, CH₂ or a heteroatom, for example O, Suitably, X is a heteroatom (typically 0) or CH or CH₂, and one of Y and Z is preferably CH or CH₂.

Ideally, X is O, n=0 or 1, and at least one of Y or Z is C═O.

The B or C ring may be functionalized with a binding agent for a target that is preferentially expressed on tumour vasculature or an anti-angiogenesis agent. The binding agent W, which may be attached to the B or C ring via a linker, for example an alkyl or aryl linker, is generally selected from an APA substrate, an APA inhibitor, an APN substrate, an APA inhibitor, an alkaline phosphatase substrate, a hydroxamic acid, or an anti-angiogenic drug.

Thus, the invention provides compounds according to the invention that have dual tubulin binding and anti-angiogenic activity. In these compounds, at least one of X, Y and Z, or a substituent on the C-ring, comprises an APA or APN inhibitor, a hydroxamic acid, or an anti-angiogenic agent. Thus, in one embodiment, at least one of X, Y and Z is selected from C(L)W, C(H)LW, C(LW)₂, N(LW), C═N(LW), C═C(LW)₂, and in which L is absent or any linker, and W is selected from an APA or APN inhibitor or hydroxamic acid. In a separate embodiment, at least one of R₁ and R₂ is an aromatic or heterocyclic ring structure having at least one substituent that is (L)W, in which L is absent or any linker, typically O or NH, and W is selected from an APA or APN inhibitor, hydroxamic acid, or an anti-angiogenic agent.

The invention also provides tubulin binding agents in a pro-drug form, in which the C-ring is functionalised with a bulky substituent, for example an amino acid, a phosphate group, a peptide, or an APA or APN inhibitor, in which the tubulin binding activity of the compound is abrogated until the bulky substituent is cleaved from the compound, whereupon the compound is activated. The bulky substituent can be chosen such that it is a substrate for an enzyme that is preferentially expressed at a target site, for example tumour vasculature. In this regard, the enzymes APA and APN are highly expressed at sites of tumour vasculature and in certain tumour cells, especially in solid tumours such as prostate tumours; thus, if an APN substrate such as a neutral amino acid is chosen as the substituent, this will result in increased activation of the prodrug at sites of tumour vasculature. Likewise, if an APA substrate such as an acidic amino acid is chosen as the substituent, this will also result in increased activation of the prodrug at sites of tumour vasculature.

Thus, the invention also provides compounds of the invention in which at least one of R₁ and R₂ is an aromatic or heterocyclic ring structure having at least one substituent that is (L)W, in which L is absent or is O or NH and W is selected from a bulky substituent, such as an APA or APN substrate, an APA or APN inhibitor, a hydroxamic acid, or a phosphate.

In one embodiment, the compound comprises a bulky substituent on the C ring that is susceptible to hydrolysis under certain pH conditions, such as physiological pH. Typically, the C-ring is functionalised with a L(W) substituent, in which L is an ester or amide (preferably ester) linkage and W is a hydroxamic acid. These molecules are susceptible to cleavage in-vivo when the molecule is exposed to physiological pH whereby the tubulin binding activity of the compound is activated. Prior to hydrolysis the compound is tubulin-binding inactive.

The Applicants have surprisingly found that the tubulin binding activity of the compounds of the invention is enhanced when the C-ring is functionalized with one or more lower alkoxy groups, preferably methoxy or ethoxy. Preferably, the lower alkoxy group is attached to the C-ring structure at the para position relative to the point of attachment of C-ring onto the B-ring. Suitably, the C-ring is functionalized with at least one amino or hydroxyl group.

Ideally, the C-ring is functionalized with both a lower alkoxy group (ideally at the para position) and an amino or hydroxyl group. Thus, at least one of R₁ and R₂ is an aromatic or heterocyclic ring structure having at least one substituent that is a lower alkoxy group, preferably in a para position relative to the point of attachment of C-ring onto the B-ring, and/or at least one substituent that is a hydroxyl or amino group.

The compounds of the invention optionally have a general formula II or III:

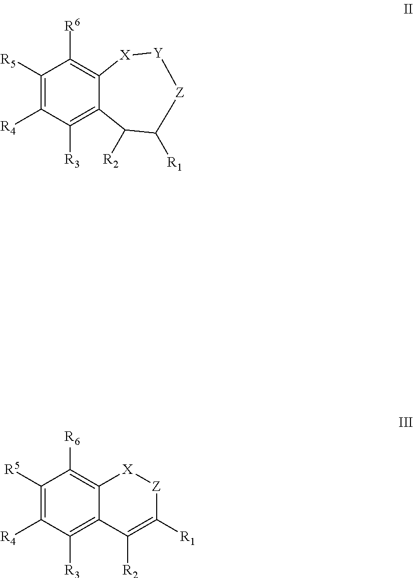

Suitably, the compounds of the invention may have the general formula IA or IB:

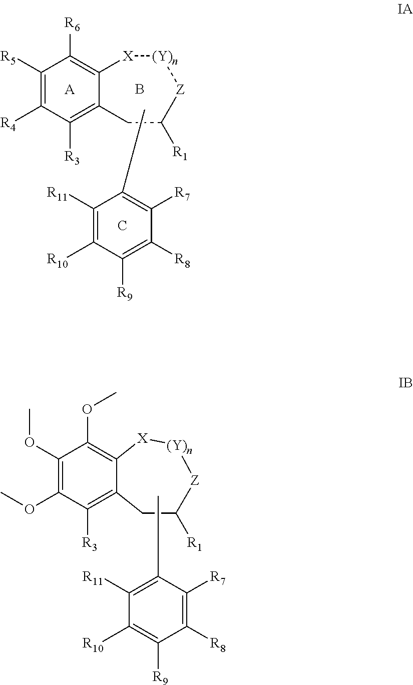

in which X, Y, Z, n, R₁, and R₃ to R₆ are as defined previously, and R₇ to R₁₁ are each, independently, selected from the groups consisting of H, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkoxycarbonylaminohydroxyl aminocarbonyl, alkylthiocarbonyl, alkoxy, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl, and ureido), amidino, imino, sulfhydryl, alkylthio, arylthio, thiocarboxylate, sulphate, sulfonato, sulfamoyl, sulfonamido, nitro (including nitrile), trifluoromethyl, isocyanate, isothiocyanate, azido, heterocyclyl, and L(W).

Typically, the compounds of the invention have the general formula IIA or IIB:

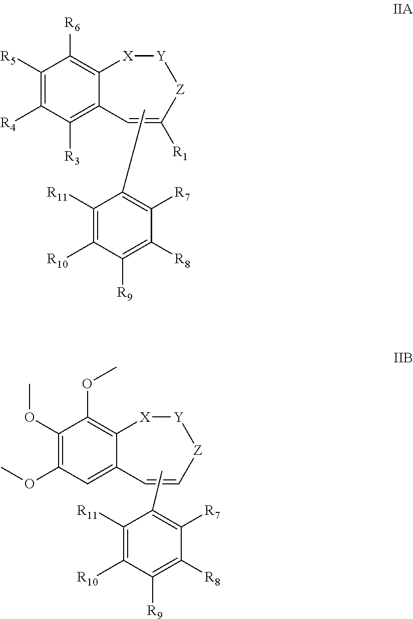

in which X, Y, Z, R₁, R₃ to R₆, and R₇ to R₁₁ are as defined previously.

Ideally, the compounds of the invention have the general formula IIIA or IIIB:

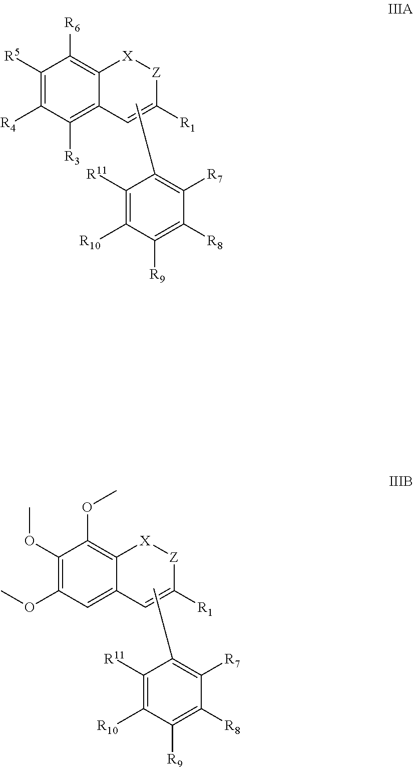

in which X, Z, R₁, R₃ to R₆, and R₇ to R₁₁ are as defined previously.

Suitably, at least one of R₇ to R₁₁ is a lower alkoxy group. Preferably, at least one of R₇ to R₁₁ is selected from OH, NH₂, W or L(W). Ideally, at least one of R₇ to R₁₁ is a lower alkoxy group and at least one of R₇ to R₁₁ is selected from OH, NH₂, W or L(W).

Preferably, R₉ is a lower alkoxy group and R₈ and R₇ is selected from OH, NH₂, W and L(W).

Typically, at least one of X, Y or Z is C═O, at least one of R₇ to R₁₁ is a lower alkoxy group, at least one of R₇ to R₁₁ is a hydroxyl or amino group, and wherein the remainder of R₇ to R₁₁ are H.

Preferably, at least one of X, Y and Z is selected from C(L)W, C(H)LW, C(LW)₂, N(LW), C═N(LW), C═C(LW)₂, in which L is absent or any linker, and in which W is selected from an APA or APN inhibitor or an anti-angiogenic agent.

In one embodiment, at least one of R₁ and R₂ is an aromatic or heterocyclic ring structure having at least one substituent that is (L)W, in which L is absent or any linker such as O or NH and W is selected from an APA or APN substrate or inhibitor, a hydroxamic acid, or an alkaline phosphatase substrate. Preferably, the at least one substituent is an APA or APN inhibitor or a hydroxamic acid.

Generally, when the substituted or unsubstituted aromatic ring structure is attached to the B-ring of formula I, II or III (via R₁ or R₂), then at least two, and preferably three of R₃ to R₆ will consist of a lower alkoxy group, ideally a methoxy, methylenedioxy or ethoxy group. Ideally, three lower alkoxy groups are attached at R₄ to R₆. With this type of structure, then the aromatic ring structure (the C-ring—e below) will preferably be substituted with at least one lower alkoxy group (ideally a methoxy group), and preferably also a hydroxyl, amine or thiol group (ideally a hydroxyl or amine group). Ideally,

In one embodiment of the compounds of the invention, the substituted or unsubstituted aromatic ring structure is a phenyl ring of general Formula IV in which R₇ to R₁₁ are as defined above, and ideally are each, independently, selected from the groups consisting of H, halogen, lower alkyl, lower alkoxy, hydroxyl, amine, thiol and a binding agent for a target that is preferentially expressed on vasculature undergoing angiogenesis, and not expressed on quiescent vasculature. Ideally, the binding agent is selected from an APA, APN or integrin binding agent, suitably an APN inhibitor or substrate, an APA inhibitor or substrate, or an integrin antagonist.

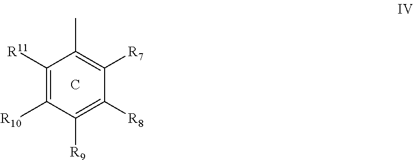

Typically, R₉ is a lower alkoxy, suitably a methoxy group and R₈ is typically R₁₉, OH or NH₂. Suitably, R₇, R₁₀ and R₁₁ are H.

In yet another embodiment, R₁₀ may be R₁₉, OH or NH₂, and R₉ may be a lower alkoxy, typically a methoxy group. Suitably, R₈ is OH. In this case, R₇, R₁₀ and R₁₁ may be H.

In the compounds of the invention, the substituted or unsubstituted aromatic ring structure may be an aromatic ring structure of general Formula V below in which R₇ to R₁₃ are each, independently, selected from the groups consisting of W, L(W), H, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkoxycarbonylaminohydroxyl aminocarbonyl, alkylthiocarbonyl, alkoxy, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl, and ureido), amidino, imino, sulfhydryl, alkylthio, arylthio, thiocarboxylate, sulphate, sulfonato, sulfamoyl, sulfonamido, nitro (including nitrile), trifluoromethyl, isocyanate, isothiocyanate, azido, heterocycle, and ideally are selected from the groups consisting of H, halogen, lower alkyl, lower alkoxy, hydroxyl, amine, thiol and a binding agent for a target that is preferentially expressed on vasculature undergoing angiogenesis, and not expressed on quiescent vasculature. Ideally, R₇ to R₁₃ are each H.

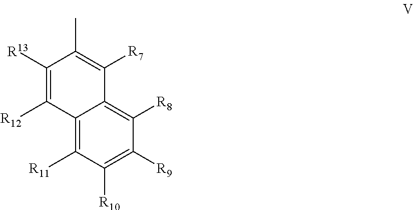

Ideally, the aromatic ring structure of formula V consists of naphthalene, however it may also consist of a substituted naphthalene ring structure.

In the compounds of the invention, one of R₁ and R₂ may be a phenyl ring of general Formula IV in which R₇ to R₁₁ are each, independently, selected from the groups consisting of H, halogen, lower alkyl, lower alkoxy, hydroxyl, amine, thiol, and a binding agent for a target that is preferentially expressed on vasculature undergoing angiogenesis, and not expressed on quiescent vasculature, or an aromatic ring structure of general Formula V in which R₇ to R₁₃ are each, independently, selected from the groups consisting of H, halogen, lower alkyl, lower alkoxy, hydroxyl, amine, thiol, and a binding agent for a target that is preferentially expressed on vasculature undergoing angiogenesis, and not expressed on quiescent vasculature. Ideally, at least three of R₃ to R₆ are alkoxy groups, suitably methoxy groups. Ideally, R₄ to R₆ are each alkoxy groups, preferably methoxy groups. Typically, when one of R₁ and R₂ is a phenyl ring of general formula IV, R₇ to R₁₁ are each, independently, selected from the groups consisting of H, lower alkoxy, hydroxyl, amine, thiol, and a binding agent for a target that is preferentially expressed on vasculature undergoing angiogenesis, and not expressed on quiescent vasculature. In a preferred embodiment, R₂ is the phenyl group of general formula I and R₁ is H.

Suitably, at least four of R₁ to R₁₃ are alkoxy groups. Generally, when one of R₇ to R₁₁ is a hydroxyl or amine group, at least two of R₄ to R₆ are alkoxy groups, and wherein when one of R₄ to R₆ is a hydroxyl or amine group, at least two of R₇ to R₁₁ are alkoxy groups.

Ideally, R₄, R₅ and R₆ are lower alkoxy, especially methoxy groups.

In a preferred embodiment of the invention, Z is C═O. In another embodiment, Y is C═O.

In one embodiment of the invention, one of R₁ and R₂, ideally R₂, is selected from the group consisting general formulae VI, VII, and VIII:

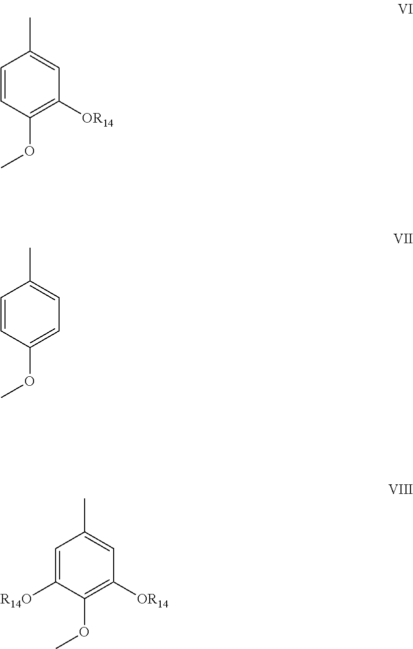

wherein R₁₄ is typically selected from the group consisting of H and a binding agent for a target that is preferentially expressed on vasculature undergoing angiogenesis, and not expressed on quiescent vasculature. Generally, R₁₄ is generally H.

In one embodiment of the invention, one of R₁ and R₂, especially R₂, is a bicyclic ring of general Formula V, such as for example naphthalene or a substituted naphthalene derivative.

### Tubulin Binding Agents

In one embodiment, the invention provides tubulin binding compounds according to the invention that typically have no dual activity. The compounds are based on the combretastatin A-4 skeletal structure having a tubulin-binding pharmacophore comprising two fused rings (A and B rings) in which the B ring is substituted with (a) an aromatic ring structure (C ring) and (b) a second substituent/functional group that comes off the B ring. The A-ring is functionalized with three lower alkoxy groups at the R₄ to R₆ positions, which has been shown to provide improved tubulin binding activity compared to similar structures functionalized at the R₃ to R₅ position. Further, the B-ring is substituted with a second functional group, that confers flexibility and functional diversity on the compound. The tubulin binding compound is suitably of general formula I:

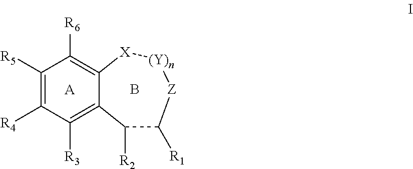

or a pharmaceutically acceptable salt thereof, wherein a dashed line indicates a single or double bond, and wherein:
   n=0, 1 or 2 (preferably 0 or 1);
   X, Y and Z are each, independently, selected from the group consisting of a heteroatom, CH, CH₂, S═O, C═O, C═S, C(R), C(H)R, C(R)₂, N(R), C═NR, C═C(R)₂, in which R is selected from the groups consisting of H, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkoxycarbonylaminohydroxyl, aminocarbonyl, alkylthiocarbonyl, alkoxy, phosphate, phosphonato, phosphinato, cyano, amino including alkyl amino, dialkylamino, arylamino, diarylamino, and alkylarylamino, acylamino including alkylcarbonylamino, arylcarbonylamino, carbamoyl, and ureido, amidino, imino, sulfhydryl, alkylthio, arylthio, thiocarboxylate, sulphate, sulfonato, sulfamoyl, sulfonamido, nitro including nitrile, trifluoromethyl, isocyanate, isothiocyanate, azido, or a heterocycle,
   R₁, R₂, and R₃ are each, independently, any substituent, selected from the groups consisting of H, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkoxycarbonylaminohydroxyl, aminocarbonyl, alkylthiocarbonyl, alkoxy, phosphate, phosphonato, phosphinato, cyano, amino including alkyl amino, dialkylamino, arylamino, diarylamino, and alkylarylamino, acylamino including alkylcarbonylamino, arylcarbonylamino, carbamoyl, and ureido, amidino, imino, sulfhydryl, alkylthio, arylthio, thiocarboxylate, sulphate, sulfonato, sulfamoyl, sulfonamido, nitro including nitrile, trifluoromethyl, isocyanate, isothiocyanate, azido, heterocycle, or a substituted or unsubstituted aromatic or heterocyclic ring structure in which the substituents (if included) are each, independently, selected from the groups consisting of H, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkoxycarbonylaminohydroxyl aminocarbonyl, alkylthiocarbonyl, alkoxy, phosphate, phosphonato, phosphinato, cyano, amino including alkyl amino, dialkylamino, arylamino, diarylamino, and alkylarylamino, acylamino including alkylcarbonylamino, arylcarbonylamino, carbamoyl, and ureido, amidino, imino, sulfhydryl, alkylthio, arylthio, thiocarboxylate, sulphate, sulfonato, sulfamoyl, sulfonamido, nitro including nitrile, trifluoromethyl, isocyanate, isothiocyanate, azido, or a heterocycle,
   at least one of R₁ and R₂ is a substituted or unsubstituted aromatic or heterocyclic ring structure in which the substituents (if included) are each, independently, selected from the groups consisting of H, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkoxycarbonylaminohydroxyl aminocarbonyl, alkylthiocarbonyl, alkoxy, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl, and ureido), amidino, imino, sulfhydryl, alkylthio, arylthio, thiocarboxylate, sulphate, sulfonato, sulfamoyl, sulfonamido, nitro (including nitrile), trifluoromethyl, isocyanate, isothiocyanate, azido, and heterocyclyl; and
   typically R₄, R₅, and R₆ are each, independently, lower alkoxy substituents,
   with the proviso that at least one of X, Y and Z is not CH or CH2 (i.e. at least one of Z, Y and Z comprises a heteroatom or a non-hydrogen substituent that comes off the ring).

Preferably, at least one of X, Y and Z (for example one of Y or Z, and ideally Z) is a carbonyl group. The Applicant has surprisingly discovered that functionalisation of the B ring with a carbonyl group enhances the tubulin binding activity of the compound. This enhancement has been found to be further increased when the C-ring is substituted with a lower alkoxy group, preferably at a para position.

Preferably, one of X, Y and Z is CH, CH₂ or a heteroatom, for example O, Suitably, X is a heteroatom (typically 0) or CH or CH₂, and one of Y and Z is preferably CH or CH₂ or a carbonyl.

Ideally, X is O, n=0 or 1, and at least one of Y or Z is C=O.

Preferably one or both of R1 and R3 are H.

Preferably, the tubulin binding compound of the invention is of general formula IA

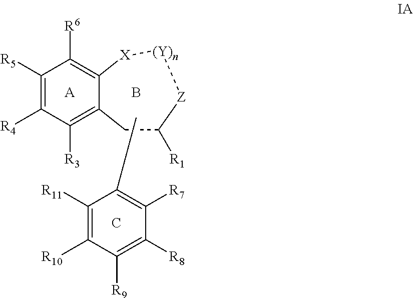

Preferably, one of R₇ to R₁₁ (preferably R₉) is a lower alkoxy group and one of R₇ to R₁₁ (preferably R₈ or R₇) is a hydroxyl or amine group.

### Dual Activity Compounds

In one embodiment, the invention provides dual activity compounds according to the invention. These compounds have tubulin binding activity and anti-angiogenesis activity. A dual active tubulin binding and anti-angiogenesis compound of the invention typically has a general formula I:

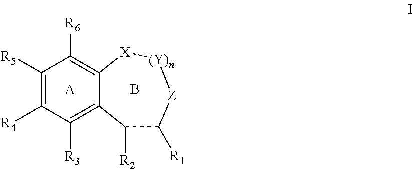

or a pharmaceutically acceptable salt thereof, wherein a dashed line indicates a single or double bond, and wherein:
   n=0 or 1;
   X, Y and Z are each, independently, selected from the group consisting of CH, CH₂, S═O, C═O, C═S, C(R), C(H)R, C(R)₂, N(R), C═NR, C═C(R)₂, C(L)W, C(H)LW, C(LW)₂, N(LW), C═N(LW), C═C(LW)₂, in which L is absent or any linker, typically selected from O, NH, CH₂O, O-alkyl, CH₂NH, and CH₂NHCOCH₂, and W is an APA or APN inhibitor, hydroxamic acid, or an anti-angiogenic drug,
   R, R₁, R₂, and R₃ are each, independently, any substituent, typically selected from the groups consisting of H, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkoxycarbonylaminohydroxyl, aminocarbonyl, alkylthiocarbonyl, alkoxy, phosphate, phosphonato, phosphinato, cyano, amino including alkyl amino, dialkylamino, arylamino, diarylamino, and alkylarylamino, acylamino including alkylcarbonylamino, arylcarbonylamino, carbamoyl, and ureido, amidino, imino, sulfhydryl, alkylthio, arylthio, thiocarboxylate, sulphate, sulfonato, sulfamoyl, sulfonamido, nitro including nitrile, trifluoromethyl, isocyanate, isothiocyanate, azido, heterocyclyl, or a substituted or unsubstituted aromatic or heterocyclic ring structure in which the substituents (if included) are each, independently, selected from the groups consisting of an APA inhibitor, APN inhibitor, or an anti-angiogenic agent, H, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkoxycarbonylaminohydroxyl aminocarbonyl, alkylthiocarbonyl, alkoxy, phosphate, phosphonato, phosphinato, cyano, amino including alkyl amino, dialkylamino, arylamino, diarylamino, and alkylarylamino, acylamino including alkylcarbonylamino, arylcarbonylamino, carbamoyl, and ureido, amidino, imino, sulfhydryl, alkylthio, arylthio, thiocarboxylate, sulphate, sulfonato, sulfamoyl, sulfonamido, nitro including nitrile, trifluoromethyl, isocyanate, isothiocyanate, azido, heterocyclyl,
   at least one of R₁ and R₂ is a substituted or unsubstituted aromatic or heterocyclic ring structure in which the substituents (if included) are each, independently, selected from the groups consisting of H, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkoxycarbonylaminohydroxyl aminocarbonyl, alkylthiocarbonyl, alkoxy, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl, and ureido), amidino, imino, sulfhydryl, alkylthio, arylthio, thiocarboxylate, sulphate, sulfonato, sulfamoyl, sulfonamido, nitro (including nitrile), trifluoromethyl, isocyanate, isothiocyanate, azido, and heterocyclyl; and
   R₄, R₅, and R₆ are preferably each, independently, lower alkoxy substituents,
   wherein: at least one of X, Y and Z (preferably Y or Z, ideally Z) is selected from C(L)W, C(H)LW, C(LW)₂, N(LW), C═N(LW), C═C(LW)₂, in which L is absent or any linker, typically selected from O, NH, CH₂O, O-alkyl, CH₂NH, and CH₂NHCOCH₂, and W is an APA or APN inhibitor, hydroxamic acid, or an anti-angiogenic drug; or at least one of R₁ and R₂ is an aromatic or heterocyclic ring structure having at least one substituent that is (L)W, in which L is absent or any linker such as O or NH and W is selected from an APA or APN inhibitor, hydroxamic acid, or an anti-angiogenic drug.

When W is hydroxamic acid, the linker may be absent or may be an alkyl or aryl group, for example X, Y or Z may be CH—(CH₂)n-CO—N—OH or C═N—O—(CH₂)n-CO—N—OH.

When W is an APA or APN inhibitor, the linker is generally selected from the group O, NH, CH2O, CH2NH.

Preferably, at least one of X, Y and Z (for example one of Y or Z, and ideally Z) is a carbonyl group. The Applicant has surprisingly discovered that functionalisation of the B ring with a carbonyl group enhances the tubulin binding activity of the compound. This enhancement has been found to be further increased when the C-ring is substituted with a lower alkoxy group, preferably at a para position relative to the point of attachment of the C-ring onto the B-ring.

Preferably, one of X, Y and Z is CH, CH₂ or a heteroatom, for example O, Suitably, X is a heteroatom (typically O) or CH or CH₂, and one of Y and Z is preferably CH or CH₂ or a carbonyl.

Ideally, X is O, n=0 or 1, and at least one of Y or Z is C═O.

Preferably one or both of R₁ and R₃ are H.

In embodiment, both the B and C ring are each, independently, substituted with an APA inhibitor, an APN inhibitor or an anti-angiogenic agent.

Preferably, the dual active tubulin binding and anti-angiogenesis compound of the invention has a general formula IA:

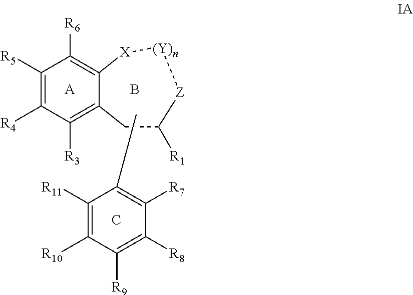

Preferably, one of R₇ to R₁₁ (preferably R₉) is a lower alkoxy group and one of R₇ to R₁₁ (preferably R₈ or R₇) is a hydroxyl or amine group.

### Pro-Drugs

In one embodiment, the tubulin binding compounds of the invention are provided in a pro-drug form in which the tubulin binding activity of the compound is abrogated until the compound is activated. The Applicant has surprisingly discovered that functionalisation of the C ring system of the tubulin binding compounds with a bulky substituent such as an amino acid abrogates the tubulin binding activity of the compound, and that removal of the bulky substituent activates the compound. The Applicant has employed this discovery to design pro-drugs that are (a) susceptible of being activated at a target site, by functionalisation of the C-ring system with a binding agent for a target that is preferentially expressed on vasculature undergoing angiogenesis, and not expressed on quiescent vasculature, and (b) susceptible to cleavage at physiological pH, for example by employing an ester linker susceptible to hydrolysis in-vivo. Pro-drugs of type (a) include binding agents for APA or APN enzymes, especially APN enzymes, that are preferentially expressed on tumour vasculature and some tumour cells.

Thus, the pro-drug suitably has a general formula I:

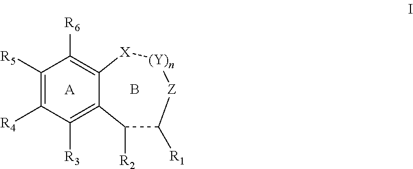

or a pharmaceutically acceptable salt thereof, wherein a dashed line indicates a single or double bond, and wherein:
   n=0, 1 or 2;
   X, Y and Z are each, independently, selected from the group consisting of CH, CH₂, S═O, C═O, C═S, C(R), C(H)R, C(R)₂, N(R), C═NR, C═C(R)₂;
   R, R₁, R₂, and R₃ are each, independently, any substituent, typically selected from the groups consisting of H, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkoxycarbonylaminohydroxyl, aminocarbonyl, alkylthiocarbonyl, alkoxy, phosphate, phosphonato, phosphinato, cyano, amino including alkyl amino, dialkylamino, arylamino, diarylamino, and alkylarylamino, acylamino including alkylcarbonylamino, arylcarbonylamino, carbamoyl, and ureido, amidino, imino, sulfhydryl, alkylthio, arylthio, thiocarboxylate, sulphate, sulfonato, sulfamoyl, sulfonamido, nitro including nitrile, trifluoromethyl, isocyanate, isothiocyanate, azido, heterocyclyl, or a substituted or unsubstituted aromatic or heterocyclic ring structure in which the substituents (if included) are each, independently, selected from the groups consisting of H, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkoxycarbonylaminohydroxyl aminocarbonyl, alkylthiocarbonyl, alkoxy, phosphate, phosphonato, phosphinato, cyano, amino including alkyl amino, dialkylamino, arylamino, diarylamino, and alkylarylamino, acylamino including alkylcarbonylamino, arylcarbonylamino, carbamoyl, and ureido, amidino, imino, sulfhydryl, alkylthio, arylthio, thiocarboxylate, sulphate, sulfonato, sulfamoyl, sulfonamido, nitro including nitrile, trifluoromethyl, isocyanate, isothiocyanate, azido, heterocyclyl; and
   R₄, R₅, and R₆ are each, independently, lower alkoxy substituents,
   wherein at least one of R₁ and R₂ is a substituted aromatic or heterocyclic ring structure in which the at least one substituent is L(W), in which L is absent or any linker and W is a bulky substituent, for example a peptide or amino acid (i.e. an APA inhibitor or substrate or an APN inhibitor or substrate).

Preferably, W is selected from an APA or APN substrate, and ideally is a neutral or acidic amino acid.

For pH sensitive activation, the linker should be susceptible to hydrolysis at physiological pH, for example an amide or ester linker.

Preferably, at least one of X, Y and Z (for example one of Y or Z, and ideally Z) is a carbonyl group. The Applicant has surprisingly discovered that functionalisation of the B ring with a carbonyl group enhances the tubulin binding activity of the compound. This enhancement has been found to be further increased when the C-ring is substituted with a lower alkoxy group, preferably at a para position.

Preferably, one of X, Y and Z is CH, CH₂ or a heteroatom, for example O, Suitably, X is a heteroatom (typically 0) or CH or CH₂, and one of Y and Z is preferably CH or CH₂ or a carbonyl.

Ideally, X is O, n=0 or 1, and at least one of Y or Z is C═O.

Preferably one or both of R1 and R3 are H.

Preferably, the pro-drug compound of the invention has a general formula IIA:

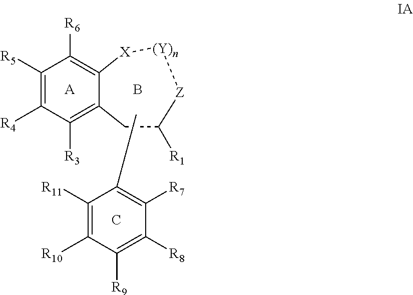

Preferably, one of R₇ to R₁₁ (preferably R₉) is a lower alkoxy group and one of R₇ to R₁₁ (preferably R₈ or R₇) is a hydroxyl or amine group.

### Intermediates

The invention also provides intermediates suitable for preparing the compounds of the invention. An intermediate suitable for preparing a compound of the invention has a general formula X:

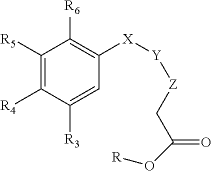

in which:
   X is a heteroatom or CH2;
   Y and Z are each, independently, CH₂ or a carbonyl group
   R₄ to R₆ are lower alkoxy groups;
   R₃ is any substituent; and
   R is an alkyl group.

The invention also relates to a compound selected from the group consisting of Compounds 1 to 56 of Table 1, or a pharmaceutically acceptable salt thereof.

TABLE 1
Compounds

| | |
|---|---|
| 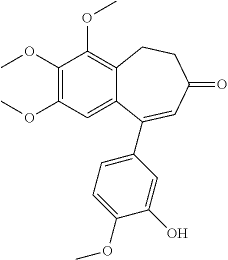 [SMILES: COc1ccc(C2=CC(=O)CCc3c2cc(OC)c(OC)c3OC)cc1O] | 1 |
| 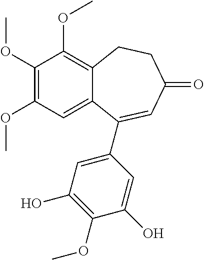 [SMILES: COc1cc2c(c(OC)c1OC)CCC(=O)C=C2c1cc(O)c(OC)c(O)c1] | 2 |
| 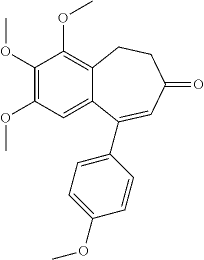 [SMILES: COc1ccc(C2=CC(=O)CCc3c2cc(OC)c(OC)c3OC)cc1] | 3 |
| 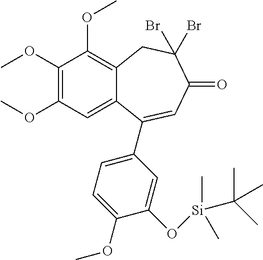 [SMILES: COc1ccc(C2=CC(=O)C(Br)(Br)Cc3c2cc(OC)c(OC)c3OC)cc1O[Si](C)(C)C(C)(C)C] | 4 |
| 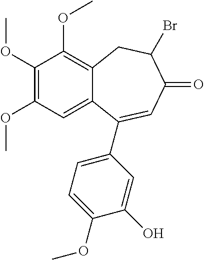 [SMILES: COc1ccc(C2=CC(=O)C(Br)Cc3c2cc(OC)c(OC)c3OC)cc1O] | 5 |
| 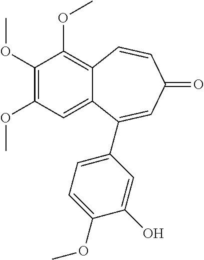 [SMILES: COc1ccc(-c2cc(=O)ccc3c(OC)c(OC)c(OC)cc23)cc1O] | 6 |
| 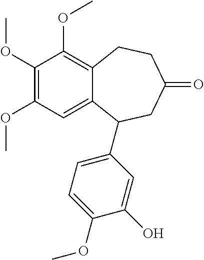 [SMILES: COc1ccc(C2CC(=O)CCc3c2cc(OC)c(OC)c3OC)cc1O] | 7 |
| 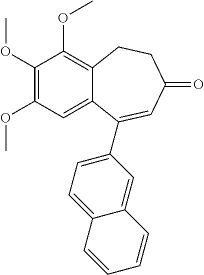 [SMILES: COc1cc2c(c(OC)c1OC)CCC(=O)C=C2c1ccc2ccccc2c1] | 8 |
| 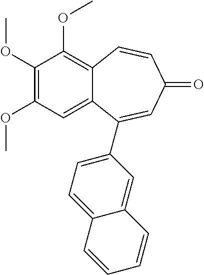 [SMILES: COc1cc2c(-c3ccc4ccccc4c3)cc(=O)ccc2c(OC)c1OC] | 9 |
| 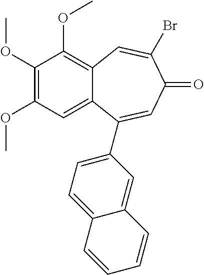 [SMILES: COc1cc2c(-c3ccc4ccccc4c3)cc(=O)c(Br)cc2c(OC)c1OC] | 10 |
| 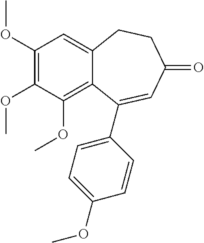 [SMILES: COc1ccc(C2=CC(=O)CCc3cc(OC)c(OC)c(OC)c32)cc1] | 11 |
| 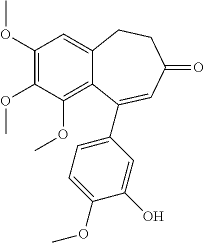 [SMILES: COc1ccc(C2=CC(=O)CCc3cc(OC)c(OC)c(OC)c32)cc1O] | 12 |
| 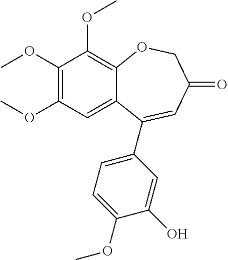 [SMILES: COc1ccc(C2=CC(=O)COc3c2cc(OC)c(OC)c3OC)cc1O] | 13 |
| 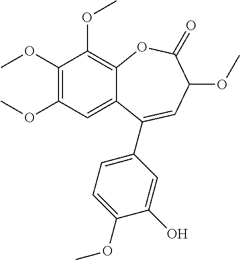 | 14 |
| 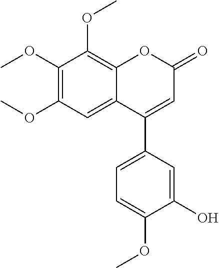 [SMILES: COc1ccc(-c2cc(=O)oc3c(OC)c(OC)c(OC)cc23)cc1O] | 15 |
| 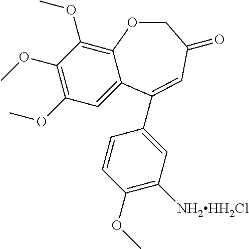 | 16 |
| 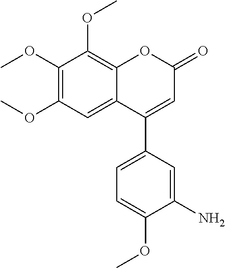 [SMILES: COc1ccc(-c2cc(=O)oc3c(OC)c(OC)c(OC)cc23)cc1N] | 17 |
| 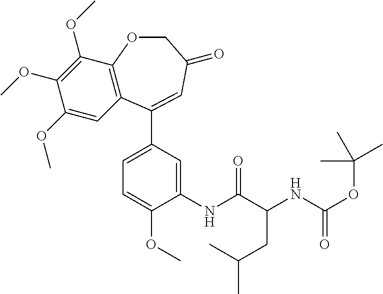 [SMILES: COc1ccc(C2=CC(=O)COc3c2cc(OC)c(OC)c3OC)cc1NC(=O)C(CC(C)C)NC(=O)OC(C)(C)C] | 18 |
| 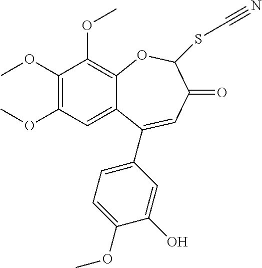 [SMILES: COc1ccc(C2=CC(=O)C(SC#N)Oc3c2cc(OC)c(OC)c3OC)cc1O] | 19 |
| 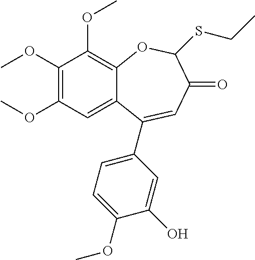 [SMILES: CCSC1Oc2c(cc(OC)c(OC)c2OC)C(c2ccc(OC)c(O)c2)=CC1=O] | 20 |
| 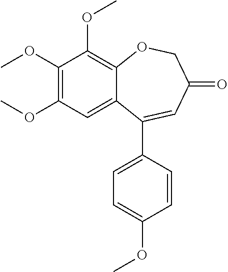 [SMILES: COc1ccc(C2=CC(=O)COc3c2cc(OC)c(OC)c3OC)cc1] | 21 |
| 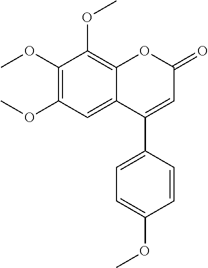 [SMILES: COc1ccc(-c2cc(=O)oc3c(OC)c(OC)c(OC)cc23)cc1] | 22 |
| 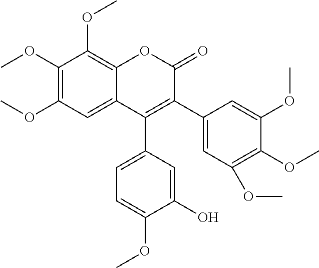 | 23 |
| 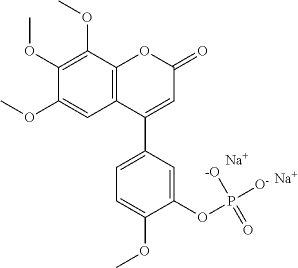 | 24 |
| 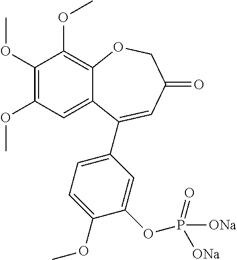 | 25 |
| 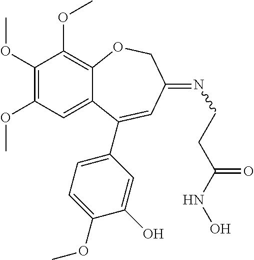 [SMILES: COc1ccc(C2=CC(=NCCC(=O)NO)COc3c2cc(OC)c(OC)c3OC)cc1O] | 26 |
| 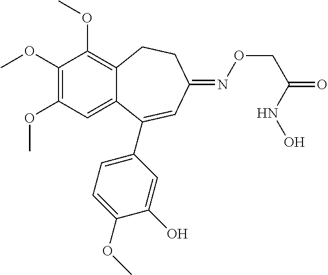 [SMILES: COc1ccc(C2=C/C(=N/OCC(=O)NO)CCc3c2cc(OC)c(OC)c3OC)cc1O] | 27 |
| 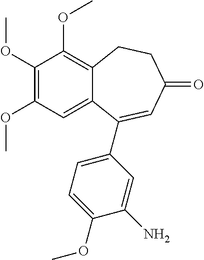 [SMILES: COc1ccc(C2=CC(=O)CCc3c2cc(OC)c(OC)c3OC)cc1N] | 28 |
| 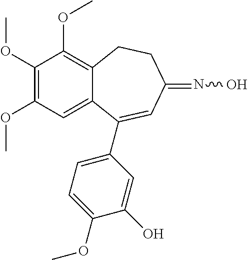 [SMILES: COc1ccc(C2=CC(=NO)CCc3c2cc(OC)c(OC)c3OC)cc1O] | 29 |
| 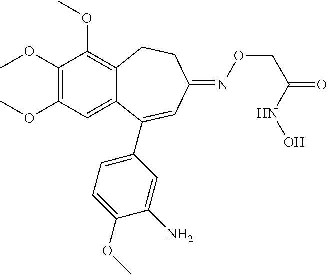 [SMILES: COc1ccc(C2=C/C(=N/OCC(=O)NO)CCc3c2cc(OC)c(OC)c3OC)cc1N] | 30 |
| 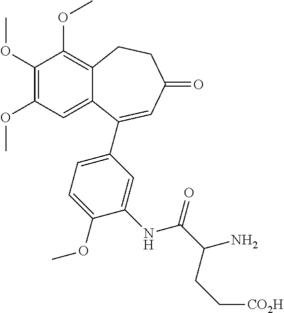 [SMILES: COc1ccc(C2=CC(=O)CCc3c2cc(OC)c(OC)c3OC)cc1NC(=O)C(N)CCC(=O)O] | 31 |
| 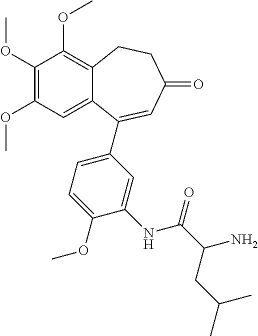 [SMILES: COc1ccc(C2=CC(=O)CCc3c2cc(OC)c(OC)c3OC)cc1NC(=O)C(N)CC(C)C] | 32 |
| 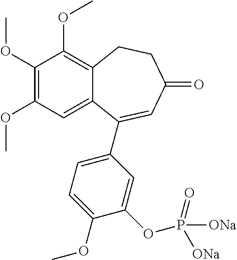 | 33 |
| 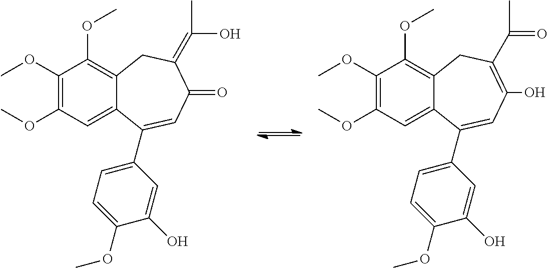 [SMILES: COc1ccc(C2=CC(=O)/C(=C(/C)O)Cc3c2cc(OC)c(OC)c3OC)cc1O ; COc1ccc(C2=CC(O)=C(C(C)=O)Cc3c2cc(OC)c(OC)c3OC)cc1O] | 34 |
| 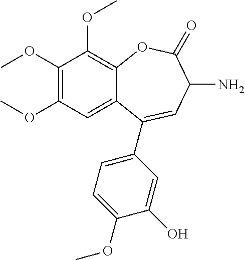 [SMILES: COc1ccc(C2=CC(N)C(=O)Oc3c2cc(OC)c(OC)c3OC)cc1O] | 35 |
| 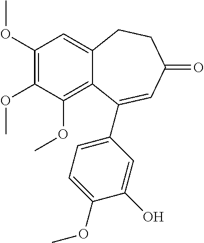 [SMILES: COc1ccc(C2=CC(=O)CCc3cc(OC)c(OC)c(OC)c32)cc1O] | 36 |
| 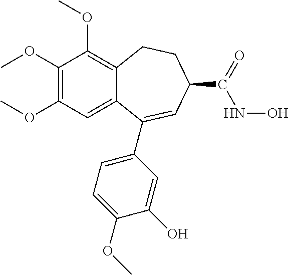 [SMILES: COc1ccc(C2=C[C@H](C(=O)NO)CCc3c2cc(OC)c(OC)c3OC)cc1O] | 37 |
| 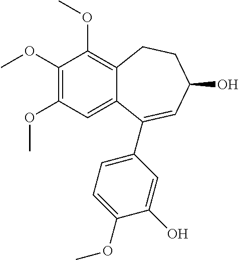 [SMILES: COc1ccc(C2=C[C@H](O)CCc3c2cc(OC)c(OC)c3OC)cc1O] | 38 |
| 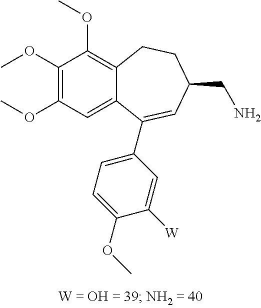 | |
| 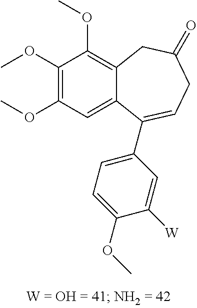 | |
| 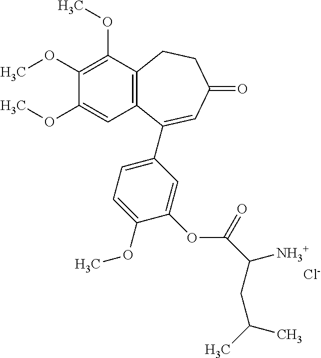 [SMILES: COc1ccc(C2=CC(=O)CCc3c2cc(OC)c(OC)c3OC)cc1OC(=O)C([NH3+])CC(C)C ; [Cl-]] | 43 |
| 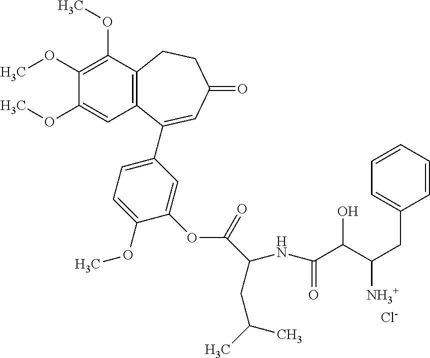 [SMILES: COc1ccc(C2=CC(=O)CCc3c2cc(OC)c(OC)c3OC)cc1OC(=O)C(CC(C)C)NC(=O)C(O)C([NH3+])Cc1ccccc1 ; [Cl-]] | 44 |
| 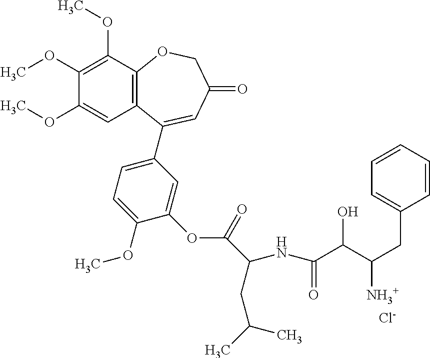 [SMILES: COc1ccc(C2=CC(=O)COc3c2cc(OC)c(OC)c3OC)cc1OC(=O)C(CC(C)C)NC(=O)C(O)C([NH3+])Cc1ccccc1 ; [Cl-]] | 45 |
| 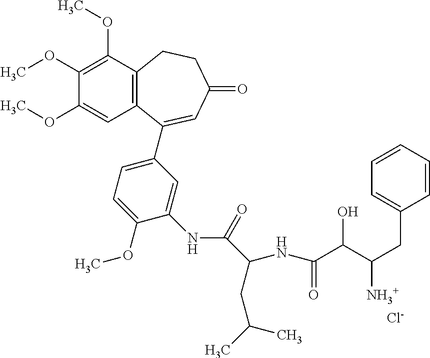 [SMILES: COc1ccc(C2=CC(=O)CCc3c2cc(OC)c(OC)c3OC)cc1NC(=O)C(CC(C)C)NC(=O)C(O)C([NH3+])Cc1ccccc1 ; [Cl-]] | 46 |
| 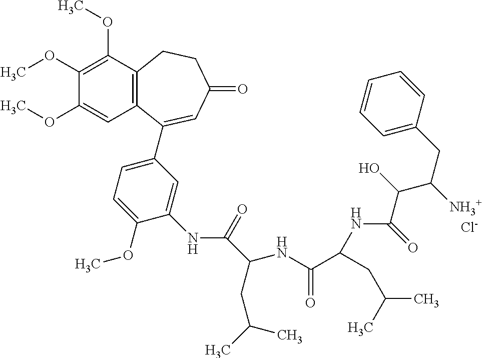 [SMILES: COc1ccc(C2=CC(=O)CCc3c2cc(OC)c(OC)c3OC)cc1NC(=O)C(CC(C)C)NC(=O)C(CC(C)C)NC(=O)C(O)C([NH3+])Cc1ccccc1 ; [Cl-]] | 47 |
| 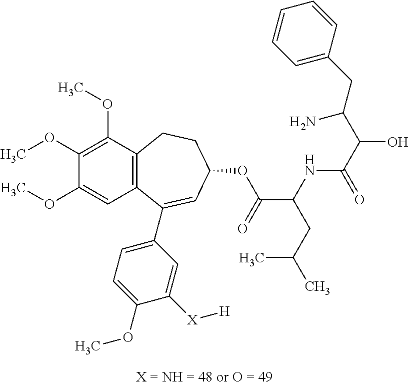 | |
| 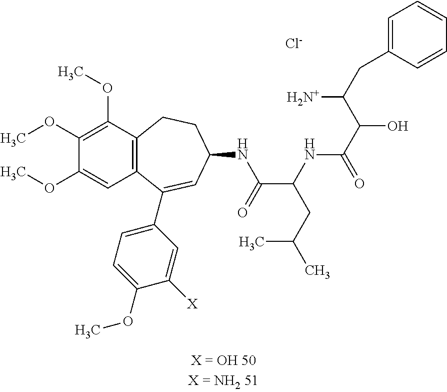 | |
| 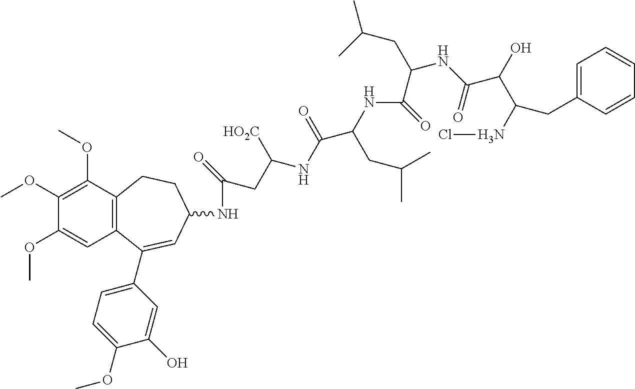 [SMILES: COc1ccc(C2=CC(NC(=O)CC(NC(=O)C(CC(C)C)NC(=O)C(CC(C)C)NC(=O)C(O)C(Cc3ccccc3)NCl)C(=O)O)CCc3c2cc(OC)c(OC)c3OC)cc1O] | 52 |
| 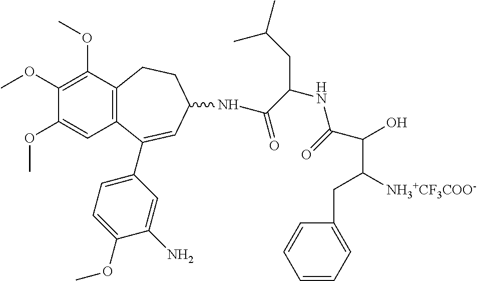 | 53 |
| 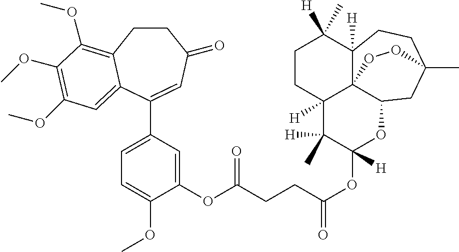 | 54 |
| 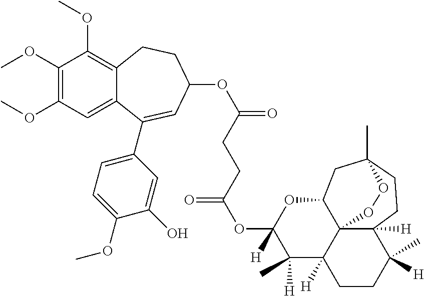 [SMILES: COc1ccc(C2=CC(OC(=O)CCC(=O)O[C@@H]3O[C@@H]4C[C@@]5(C)CC[C@H]6[C@H](C)CC[C@@H]([C@H]3C)[C@@]46OO5)CCc3c2cc(OC)c(OC)c3OC)cc1O] | 55 |
| 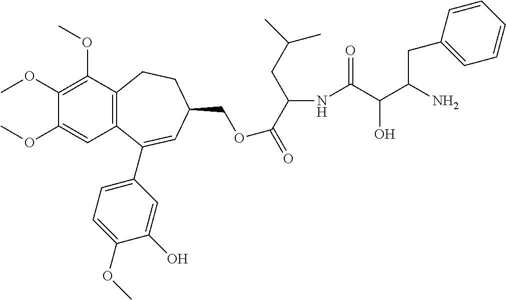 [SMILES: COc1ccc(C2=C[C@H](COC(=O)C(CC(C)C)NC(=O)C(O)C(N)Cc3ccccc3)CCc3c2cc(OC)c(OC)c3OC)cc1O] | 56 |

The invention also relates to the compounds substantially as herein described with reference to the accompanying Description.

The invention also relates to the compounds substantially as herein described with reference to the accompanying Figures.

The invention also relates to a pharmaceutical composition comprising a compound or pro-drug of the invention, in combination with a suitable pharmaceutical carrier.

The invention also relates to a use of a compound or pro-drug of the invention as a medicament.

The invention also relates to a method of treating or preventing cancer in a mammal, especially a human, comprising a step of administering a suitable amount of a compound or a pro-drug to the mammal.

The invention also relates to a method of treatment or prevention of cancer in a mammal, especially a human, by inhibiting angiogenesis at the tumour site comprising a step of administering a suitable amount of a compound or pro-drug of the invention to the mammal.

The invention also relates to a method of treatment or prevention of cancer in a mammal, especially a human, by inhibiting tubulin assembly and angiogenesis at the tumour site comprising a step of administering a suitable amount of a compound or pro-drug of the invention to the mammal.

The invention also relates to a method of inhibiting angiogenesis in a cell, tissue, organ or individual comprising a step of treating the cell, tissue, organ or individual with a compound of the invention. The invention also relates to a method of preventing or treating a disease or condition characterised by an increased level of angiogenesis in an individual, comprising a step of administering to the individual a therapeutic amount of a compound of the invention.

The invention also relates to a method of inhibiting angiogenesis in a cell, tissue, organ or individual comprising a step of treating the cell, tissue, organ or individual with a compound of the invention in which the compound of the invention inhibits mast cell degranulation and release of pro-angiogenic mediators via anti-IgE mediated and non-anti-IgE mediated processes.

The invention also relates to a method of inhibiting or preventing the release of pro-angiogenic mediators from mast cells comprising the step of treating the cells with a compound of the invention.

The invention also relates to a drug-eluting stent comprising, or capable of in-vivo release of, a compound of the invention.

### BRIEF DESCRIPTION OF THE FIGURES

FIG. 1: Tunable molecular scaffolds: (1-5) B-ring functional group diversification; (7-8) stereochemical diversification; and (9-15) ligand diversification

FIG. 2: Increase in optical density of a tubulin solution (100 μl) and DMSO blank (1 μl)

FIG. 2a: Cell cycle histograms of the gated G₀/G₁/S/G₂/M cells. HUVECs were treated for 24 h, stained with PI and analysed using flow cytometry A) and D) Control (0.1% DMSO), B) 0.5 μM 1, C) 1 μM 1, E) 0.5 μM 28 and F) 1 μM 28.

FIG. 2b: Microtubule disruption of endothelial cells. HUVECs were treated for 30 minutes and stained for α-tubulin (green) and nucleus (blue). Images were taken using an Olympus FV1000 Point Scanning Confocal Microscope (×60). A-B) Control (0.1% DMSO), C-D) 1 μM 1 and E-F) 1 μM 28.

FIG. 2c: The effect of 1 and CA-4 on endothelial cell morphology. HUVECs were exposed to the compounds for 40 minutes and photomicrographs (×10) were taken 1-h after drug washout A/E) Control (0.1% DMSO), B) 1 0.1 μM, C) 28 0.5 μM, D) 1 1 μM, F) CA-4 0.1 μM, G) CA-4 0.5 μM and H) CA-4 1 μM.

FIG. 2d The reversible effect of 28 on endothelial cells' morphology. HUVECs were exposed to the compounds for 40 minutes and photomicrographs (×10) were taken 1-h (*) and 3-h (**) after drug washout A) Control (0.1% DMSO) B) 28 0.1 μM and C) 28 0.5 μM.

FIG. 2e: The aortic rings were kept in a humidified CO₂ incubator at 37° C. and the medium was changed three times a week starting from day 3. Test compounds were added on day 8. Photomicrographs of the aortic cultures were taken under bright field microscopy using a digital camera (×4). A-B) Before the addition of test compound, C-D) 100 nM 1 (4 h) and E-F) 100 nM 1 (24 h).

FIG. 2f The aortic rings were kept in a humidified CO₂ incubator at 37° C. and the medium was changed three times a week starting from day 3. Test compounds were added on day 8. Photomicrographs of the aortic cultures were taken under bright field microscopy using a digital camera (×4). A-B) Before the addition of test compound, C-D) 100 nM 28 (4 h) and E-F) 100 nM 28 (24 h).

FIG. 2g: The aortic rings were kept in a humidified CO₂ incubator at 37° C. and the medium was changed three times a week starting from day 3. Test compounds were added on day 8. Photomicrographs of the aortic cultures were taken under bright field microscopy using a digital camera (×4). A) Control (0.1% DMSO), B) 1 nM 1, C) 10 nM 1 and D) 100 nM 1.

FIG. 2g1: The microvessel density of 1-treated cultures was obtained using Image J. The data represents mean pixel density area±SEM (n=3).

FIG. 2h: The aortic rings were kept in a humidified CO₂ incubator at 37° C. and the medium was changed three times a week starting from day 3. Test compounds were added on day 8. Photomicrographs of the aortic cultures were taken under bright field microscopy using a digital camera (×4). A) Control (0.1% DMSO), B) 10 nM 28 and C) 50 nM 28.

FIG. 2h1: The microvessel density of 28 treated cultures was obtained using Image J. The data represents mean pixel density area±SEM (n=3).

FIG. 2i: Microtubule disruption of endothelial cells. HUVECs were treated for 30 minutes and stained for α-tubulin (green) and nucleus using DAPI (blue). Images were taken using an Olympus FV1000 Point Scanning Confocal Microscope (×60). A-B) Control (0.1% DMSO), C-D) 1 μM 44.

FIG. 2j: Microtubule disruption of endothelial cells. HUVECs were treated for 30 minutes and stained for α-tubulin (green) and nucleus using DAPI (blue). Images were taken using an Olympus FV1000 Point Scanning Confocal Microscope (×60). A-B) Control (0.1% DMSO) and C) 1 μM 45.

FIG. 2k: APN inhibition of HUVECs after 2 h incubation with A) bestatin, B) 50 and C) 51. Data represents mean±SEM (n=3).

FIG. 2l: APN inhibition of PC-3 cells after 2 h incubation with A) bestatin, B) 50 and C) 51. Data represents mean±SEM (n=3).

FIG. 2m: APN inhibition by 44 of A) HUVECs and B) PC-3 cells after 2 h incubation. Data represents mean±SEM (n=3).

FIG. 2n: APN inhibition by 45 of A) HUVECs and B) PC-3 cells after 2 h incubation. Data represents mean±SEM (n=3).

FIG. 2o: The effect of 44 on endothelial cells' morphology. HUVECs were exposed to the compounds for 40 minutes and photomicrographs (×10) were taken 1-h (*) and 3-h (**) after drug washout A) Control (0.1% DMSO) B) 44 0.1 μM and C) 44 1 μM.

FIG. 2p: The effect of 45 on endothelial cells' morphology. HUVECs were exposed to the compounds for 40 minutes and photomicrographs (×10) were taken 1-h (*) and 3-h (**) after drug washout A) Control (0.1% DMSO) B) 45 0.1 μM and C) 45 1 μM.

FIG. 2q1: The aortic rings were kept in a humidified CO₂ incubator at 37° C. and the medium was changed three times a week starting from day 3. Test compounds were added on day 8. Photomicrographs of the aortic cultures were taken under bright field microscopy using a digital camera (×4). A-B) Before the addition of test compound and C-D) 1 μM 44 (24 h).

FIG. 2q2: The aortic rings were kept in a humidified CO₂ incubator at 37° C. and the medium was changed three times a week starting from day 3. Test compounds were added on day 8. Photomicrographs of the aortic cultures were taken under bright field microscopy using a digital camera (×4). A-B) Before the addition of test compound, C-D) 50 nM 45 (4 h) and E-F) 50 nM 45 (24 h).

FIG. 2r The aortic rings were kept in a humidified CO₂ incubator at 37° C. and the medium was changed three times a week starting from day 3. Test compounds were added on day 8. Photomicrographs of the aortic cultures were taken under bright field microscopy using a digital camera (×4). A-B) Before the addition of test compound, C-D) 1 μM 30 (4 h) and E-F) 1 μM 30 (24 h).

FIG. 2s: The aortic rings were kept in a humidified CO₂ incubator at 37° C. and the medium was changed three times a week starting from day 3. Test compounds were added on day 1. Photomicrographs of the aortic cultures were taken under bright field microscopy using a digital camera (×4). A) Control (0.1% DMSO), B) 10 nM 44 and C) 100 nM 44.

FIG. 2t: The microvessel density of 44-treated cultures was obtained using Image J. The data represents mean pixel density area±SEM (n=3).

FIG. 2u: The aortic rings were kept in a humidified CO₂ incubator at 37° C. and the medium was changed three times a week starting from day 3. Test compounds were added on day 1. Photomicrographs of the aortic cultures were taken under bright field microscopy using a digital camera (×4). A) Control (0.1% DMSO), B) 10 nM 45 and C) 50 nM 45.

FIG. 2v: The microvessel density of 45-treated cultures was obtained using Image J. The data represents mean pixel density area±SEM (n=3).

FIG. 2w: The aortic rings were kept in a humidified CO₂ incubator at 37° C. and the medium was changed three times a week starting from day 3. Test compounds were added on day 1. Photomicrographs of the aortic cultures were taken under bright field microscopy using a digital camera (×4). A) Control (0.1% DMSO), B) 0.1 μM 30 and C) 1 μM 30.

FIG. 2wi: The microvessel density of 30-treated cultures was obtained using Image J. The data represents mean pixel density area±SEM (n=3).

### DETAILED DESCRIPTION OF THE INVENTION

The invention provides diverse scaffolds, arising from a central three-ring design architecture which in one embodiment targets multiple events arising from interaction with microtubule dynamics and selective expression of aminopeptidases N (APN) and A (APA) on tumour angiogenic vasculature. The compounds of the invention demonstrate precise positioning of substituents on the A-ring, functional group diversification on the B-ring, and, optionally, exploitation of the C-ring for prodrug and pH-sensitive release of the tubulin binding component, and involve the design of novel synthetic methods, mediated primarily through unique bromine substitution reactions, to furnish series of ring-contracted, 4-phenyl-chromen-2-ones (“nature series”) and 5-phenyl-3H-benzo[b]oxepin-2-one series respectively. Inclusion of APN targeting groups onto the B- or C-rings of the scaffold enabled the synthesis of dual-acting hybrids and designed multiple ligands with up to 10-fold higher activity than the prototypical APN inhibitor bestatin while inclusion onto the C-ring of substrates and inhibitors of APN allows for pH-sensitive release of the tubulin-binding component. Selected compounds show potent, low nM synergistic activity, in cellular, ex vivo vascularisation models and in the APN-expressing PC-3 prostate tumour model in vivo. Moreover, specific tuning of the scaffold's A- and C-rings combined with B-ring diversity can yield similarly diverse ligand classes targeting other pathologies.

In one aspect, the present invention provides tumour angiogenesis/vasculature targeting agents (dual activity compounds) that incorporate into their design, components that have the capacity to inhibit both tubulin polymerisation and APN or APA. APN and APA are widely recognised to play pivotal roles in the process of angiogenesis. Pasqualini et al demonstrated APN to be specifically expressed in endothelial and sub-endothelial cells undergoing angiogenesis but not on normal vasculature. APN is involved in several key events in angiogenesis including breakdown of the extracellular matrix, endothelial cell migration and capillary tube formation. Recently Rangel et al demonstrated with APN null mice that APN plays an essential role in pathological angiogenesis but has no effect on vasculogenesis during foetal and embryonic development or on normal adult function. In addition, certain integrins have been shown to be specifically expressed in tumour tissue, and involved in the process of angiogenesis. APN and integrins are therefore very effective targets for inhibition of tumour angiogenesis. Tubulin also plays a vital role in angiogenesis. Microtubules, which are composed of α- and β-tubulin dimers, are essential components of the mitotic spindle and thus play an integral role in cell division. They are also involved in cellular functions such as proliferation, differentiation and apoptosis. Microtubule targeting agents are an important class of anti-tumour drugs, which inhibit EC proliferation and migration, degradation of the basement membrane and the ECM, and capillary-tube formation on Matrigel®. As well as the aforementioned anti-angiogenic effects, many of these agents also act as vascular targeting agents (VTAs) causing rapid and dramatic changes to endothelial cell morphology, which ultimately results in vascular shutdown.

The compounds of the invention are designed to target tumour angiogenesis by typically inhibiting (i) endothelial cell proliferation, (ii) endothelial cell motility, (iii) extracellular matrix breakdown, (iv) capillary tube formation and to cause tumour vasculature shutdown by altering the morphology of endothelial cells.

In this specification, the term “cancer” should be taken to mean a cancer selected from the group consisting of: fibrosarcoma; myxosarcoma; liposarcoma; chondrosarcom; osteogenic sarcoma; chordoma; angiosarcoma; endotheliosarcoma; lymphangiosarcoma; lymphangioendotheliosarcoma; synovioma; mesothelioma; Ewing's tumor; leiomyosarcoma; rhabdomyosarcoma; colon carcinoma; pancreatic cancer; breast cancer; ovarian cancer; prostate cancer; squamous cell carcinoma; basal cell carcinoma; adenocarcinoma; sweat gland carcinoma; sebaceous gland carcinoma; papillary carcinoma; papillary adenocarcinomas; cystadenocarcinoma; medullary carcinoma; bronchogenic carcinoma; renal cell carcinoma; hepatoma; bile duct carcinoma; choriocarcinoma; seminoma; embryonal carcinoma; Wilms' tumor; cervical cancer; uterine cancer; testicular tumor; lung carcinoma; small cell lung carcinoma; bladder carcinoma; epithelial carcinoma; glioma; astrocytoma; medulloblastoma; craniopharyngioma; ependymoma; pinealoma; hemangioblastoma; acoustic neuroma; oligodendroglioma; meningioma; melanoma; retinoblastoma; and leukemias. In a preferred embodiment, the cancer is selected from the group comprising: breast; cervical; prostate; and leukemias, and/or their metastases. In a most preferred embodiment, the cancer is a cancer characterized by local expression of APA or APN at a tumour site, for example a prostate cancer.

The term “binding agent for a target that is preferentially expressed on vasculature undergoing angiogenesis, and not expressed on quiescent vasculature” should be understood to mean a binding agent for an APA or APN enzyme. The term preferably means an APA inhibitor, an APA substrate, an APN inhibitor, an APN substrate, and also a hydroxamic acid moiety. Ideally, the binding agent is an aminopeptidase N (APN) inhibitor.

The term “pro-drug” in the context of the present invention refers to compounds that have no or minimal tubulin binding activity due to the presence of a bulky substituent on the C-ring, and in which removal of the bulky substituent results in activation of the compound. Examples of bulky sub stituents include an amino acid, a peptide, or any other bulky substituent such as a phosphate. Without being bound by theory, it is believed that the presence of the bulky substituent on the C-ring, especially the R₈ or R₁₀ positions on the C-ring, prevents tubulin binding.

The term “homing activity” as applied to a compound of the invention refers to the ability of the compound to preferentially accumulate at a vasculature site that is undergoing angiogenesis compared to a quiescent vasculature site. Compounds of the invention are capable of homing activity due to the compounds including in their architecture a binding agent for a protein (target) which is preferentially expressed in vasculature undergoing angiogenesis. The binding agent may be an inhibitor or antagonist, but is preferably a substrate, of the target. Ideally, the binding agent is an APA or APN substrate, which is typically attached to the C-ring via an esterase sensitive linkage.

The term “dual activity” as applied to the compounds of the invention refers to (a) tubulin binding activity and (b) anti-angiogenesis activity mediated via inhibition of APN or APA. Compounds of the invention that are capable of dual activity will generally include in their architecture an APA or APN inhibitor, expecially an APN inhibitor such as, for example, bestatin or probestin.

The term “anti-angiogenesis activity” as applied to compounds of the invention refers to the ability of the compounds to inhibit, reduce or ameliorate angiogenesis at the site of action specifically through inhibition of APN or APA enzymes, or via antagonism of the integrin receptor.

The term “anti-angiogenic agent” refers to an agent capable of ameliorating angiogenesis at a tumour site. Suitable agents include Artesunate, bevacizumab (Avastin), Sorafenib (Nexavar), Sunitinib (Sutent), Pazopanib (Votrient), Everolimus (Afinotor).

“Aminopeptidase N” (APN,CD13, EC3.4.11.2)” is a further aminopeptidase enzyme characterized by Tokioka-Terao et al, which has also been shown to be specifically expressed in endothelial and sub-endothelial cells undergoing angiogenesis but not on normal vasculature Bhagwat et al (2001). Also APN is a receptor for tumour homing peptides and especially those containing the NGR (asparagine-glycine-arginine) motif, Pasqualini et al 2000.

“Aminopeptidase N substrate” refers to a substrate of the human APN enzyme, typically a peptide substrate, examples of which include R—F(3-H)anilide (Ryan et al, Anal. Biochem. 1993; April 210(1), neutral amino acids (i.e. glycine, alanine, valine, leucine, isoleucine, methionine, phentlalanine, and tyrosine) and perhaps polar uncharged amino acids (i.e. serine, threonine, asparagine, and glutamine).

“Aminopeptidase N inhibitor” refers to inhibitors of the human APN enzyme, typically peptide or peptide-derived inhibitors, examples of which include probestin and bestatin. Examples of APN inhibitors are provided in Su et al (Expert Opin. Ther. Patents (2011) 21(8), WO2007048787, KR2006019361, US2009012153, US2009131509, WO2007057128, WO2008096276, CN101481325, CN101503373, CN101357893, CN101538311, and WO2010072327. In a preferred embodiment of the invention, the APN inhibitor is selected from bestatin, phebestin and probestin, ideally bestatin.

“Aminopeptidase A” (APA, EC 3.4.11.7) is a membrane bound zinc dependent aminopeptidase enzyme encoded by the human ENPEP gene that catalyses the cleavage of glutamic and aspartic acid residues from the N-terminus of polypeptides. The enzyme has been shown by Marchio et al (2004) to be specifically expressed in endothelial and sub-endothelial cells undergoing angiogenesis but not on normal vasculature. It is also known as glutamyl aminopeptidase.

“Aminopeptidase A substrate” refers to a substrate of the human APA enzyme, typically a peptide substrate, examples of which include amino acids, for example neutral amino acids (i.e. glycine, alanine, valine, leucine, isoleucine, methionine, phentlalanine, and tyrosine), polar uncharged amino acids (i.e. serine, threonine, asparagine, and glutamine), acidic amino acids (i.e. glutamic and aspartic acids), and analogs thereof.

The term “aminopeptidase A inhibitor” refers to inhibitors of the human APA enzyme, examples of which include EC33 and RB150 (Bodineau et al. Hypertension 2008: 51: 1318-1325), bestatin and amastatin preferably amastatin.

“Combretastatin” refers to the group of tubulin-binding agents generically described in Pettit et al., (Can. J. Chem. 1982). “Combretastatin analogs” refers to analogs of combretastatin, for example the compounds described in International Patent Application No: PCT/US2006/023251. “Combretastatin-like compounds” refers to compounds that have a tubulin-binding pharmacophore similar to combretastatin A-4, i.e. a fused A-B ring structure and an aromatic ring structure (C-ring structure) as a substituent of the B-ring.

“Tubulin Binding Agent” shall refer to a ligand of tubulin or a compound capable of binding to either Ab-tubulin heterodimers or microtubules and interfering with the assembly or disassembly of microtubules. The terms should be taken to include, but not be restricted to, combretastatin A-4 or combretastatin A-4 analogs, and also includes phenstatin molecules. Examples of tubulin binding agents include Vinca Alkyloids, including Vinblastine, Vincristine, and Taxanes such as Taxol.

“Esterase/amidase-sensitive linker/linkage” refers to a linker group that is susceptible to cleavage by an esterase or phosphatase enzyme, for example an amide link.

The binding agent for a target which is preferentially expressed on vasculature undergoing angiogenesis may be attached to the core molecule via a linker group. The linker may be any linker group, including an aryl or alkyl group. Preferred linkers include O, NH, O-alkyl, CH₂O, CH₂NH, and CH₂NHCOCH₂, CO, COO. When the binding agent is attached to the B ring, the linker will generally be O, NH, O-alkyl, CH₂O, CH₂NH, and CH₂NHCOCH₂, CO, COO. When the binding agent is attached to the C ring, the linker is also generally a N or O, although when for pH responsive compounds, the linker will generally be an esterase sensitive linkage (—O—).

“Lower alkyl” means an alkyl group, as defined below, but having from one to ten carbons, more preferable from one to six carbon atoms (eg. “C—C-alkyl”) in its backbone structure. “Alkyl” refers to a group containing from 1 to 20 carbon atoms and may be straight chained or branched. An alkyl group is an optionally substituted straight, branched or cyclic saturated hydrocarbon group. When substituted, alkyl groups may be substituted with up to four substituent groups, at any available point of attachment. When the alkyl group is said to be substituted with an alkyl group, this is used interchangeably with “branched alkyl group”. Exemplary unsubstituted such groups include methyl, ethyl, propyl, isopropyl, a-butyl, isobutyl, pentyl, hexyl, isohexyl, 4,4-dimethylpentyl, octyl, 2,2,4-trimethylpentyl, nonyl, decyl, undecyl, dodecyl, and the like. Examplary substituents may include but are not limited to one or more of the following groups: halo (such as F, CI, Br, I), Haloalkyl (such as CC13 or CF13), alkoxy, alkylthio, hydroxyl, carboxy (—COOH), alkyloxycarbonyl (—C(O)R), alkylcarbonyloxy (—OCOR), amino (—NH2), carbamoyl (—NHCOOR— or —OCONHR), urea (—NHCONHR—) or thiol (—SH). Alkyl groups as defined may also comprise one or more carbon double bonds or one or more carbon to carbon triple bonds.

“Alkoxy” refers to O-alkyl groups, wherein alkyl is as defined hereinabove, and “Lower alkoxy” refers to O-lower alkyl groups, wherein lower alkyl is as defined above. The (lower) alkoxy group is bonded to the core compound through the oxygen bridge. The (lower) alkoxy group may be straight-chained or branched; although the straight-chain is preferred. Examples include methoxy, ethyloxy, propoxy, butyloxy, t-butyloxy, i-propoxy, and the like. Preferred lower alkoxy groups contain 1-4 carbon atoms, especially preferred lower alkoxy groups contain 1-3 carbon atoms. The most preferred lower alkoxy group is methoxy or ethoxy.

“Phosphate” refers to a phosphate disalt moiety (—OP(O)(O⁻M⁺)₂, a phosphate trimester moiety (—OP(O)(OR)₂), or a phosphate ester salt moiety (—OP(O)(OR)(O⁻M⁺), where M is a salt (i.e. Na, K, Li) and each R is, independently, any suitable alkyl or branched alkyl substituent, or benzyl or aryl groups.

“Nitro” refers to a NO₂ group, and “nitrile” refers to a nitrogen atom bound to the carbon by means of a triple bond.

“Amine” refers to a primary, secondary or tertiary amine group, including an alkylamino group where one or two alkyl groups is bonded to an amino nitrogen, in which the nitrogen is the bridge connecting the alkyl group(s) to the core compound.

“Thiol” refers to an arganosulphur substituent that contains a carbon-bonded sulfhydryl group.

“Sulphonic acid” refers to a group of compounds having the general structure —S(═O)₂—OH.

“Aryl” refers to a 5- and 6-membered single ring aromatic group that may include from zero to four heteroatoms, for example benzene, pyrrole, furan, thiophene, imidazole, oxazole, thiazole, triazole, tetrazole, pyrazole, pyridine, pyrazine, pyridazine, and pyramidine, and the like. Aryl groups also include polycyclic fused aromatic groups such as naphthyl, quinolyl, indolyl, and the like. The aromatic ring can be substituted at one or more ring positions with a substituent selected from the group halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl, and ureido), amidino, imino, sulfhydryl, alkylthio, arylthio, thiocarboxylate, sulphate, sulfonato, sulfamoyl, sulfonamido, nitro, trifluoromethyl, isocyanate, isothiocyanate, azido, heterocyclyl, or an aromatic or heteroaromatic moiety.

“Aroyl” refers to —(C═O)-aryl group, wherein aryl is defined as above. The aryl group is bonded to the core compound via a carbonyl bridge.

“Halogen” means the non-metal elements of Group 17 of the periodic table, namely bromine, chlorine, fluorine, iodine and astatine.

“Salt” is a pharmaceutically acceptable salt and can include acid addition salts such as the hydrochlorides, hydrobromides, phosphates, sulphates, hydrogen sulphates, alkylsulphates, arylsulphonates, acetates, benzoates, citrates, maleates, fumarates, succinates, lactates, and tartrates; alkali metal cations such as Na, K, Li; alkali earth metal salts such as Mg or Ca; or organic amine salts. Exemplary organic amine salts are tromethamine (TRIS) salts and amino acid salts (e.g. histidine salts) of the compounds of the invention.

The bond between Y and Z in FIGS. I, IA, and IB, may be a double or single bond.

### Therapeutic Compositions and Methods of Administration

The invention provides methods of, and compositions for, treatment and prevention by administration to a subject in need of such treatment of a therapeutically or prophylactically effective amount of a compound or pro-drug of the invention. The subject may be an animal or a human, with or without an established disease.

“Treating” (or “treat”) as used herein includes its generally accepted meaning which encompasses prohibiting, preventing, restraining, and slowing, stopping or reversing progression, severity, of a resultant symptom. As such, the methods of this invention encompass both therapeutic and prophylactic administration.

“Effective amount” refers to the amount or dose of the compound, upon single or multiple dose administration to the patient, which provides the desired effect in the patient under diagnosis or treatment. An effective amount can be readily determined by the attending diagnostician, as one skilled in the art, by the use of known techniques and by observing results obtained under analogous circumstances. In determining the effective amount or dose of compound administered, a number of factors are considered by the attending diagnostician, including, but not limited to: the species of mammal; its size, age, and general health; the specific disease involved; the degree of or involvement or the severity of the disease; the response of the individual patient; the particular compound administered; the mode of administration; the bioavailabilty characteristics of the preparation administered; the dose regimen selected; the use of concomitant medication; and other relevant circumstances.

The present invention also provides pharmaceutical compositions. Such compositions comprise a therapeutically effective amount of a compound or pro-drug of the invention, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals and more particularly in humans.

The term “carrier” refers to a diluent, adjuvant, excipient, or vehicle with which the compound or pro-drug of the invention is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol and water.

The composition, if desired, can also contain minor amounts of wetting or emulsifying agents, or pH buffering agents. These compositions can take the form of solutions, suspensions, emulsion, tablets, pills, capsules, powders, sustained-release formulations and the like.

The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound or pro-drug of the invention, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to, ease pain at the, site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In the case of cancer, the amount of the therapeutic of the invention which will be effective in the treatment or prevention of cancer will depend on the type, stage and locus of the cancer, and, in cases where the subject does not have an established cancer, will depend on various other factors including the age, sex, weight, and clinical history of the subject. The amount of therapeutic may be determined by standard clinical techniques. In addition, in vivo and/or in vitro assays may optionally be employed to help predict optimal dosage ranges. The precise dose to be employed in the formulation will also depend on the route of administration, and the seriousness of the cancer, and should be decided according to the judgment of the practitioner and each patient's circumstances. Routes of administration of a therapeutic include, but are not limited to, intramuscularly, subcutaneously or intravenously. Effective doses may be extrapolated from dose-response curves derived from in vitro or animal model test systems.

### Chemical Synthesis

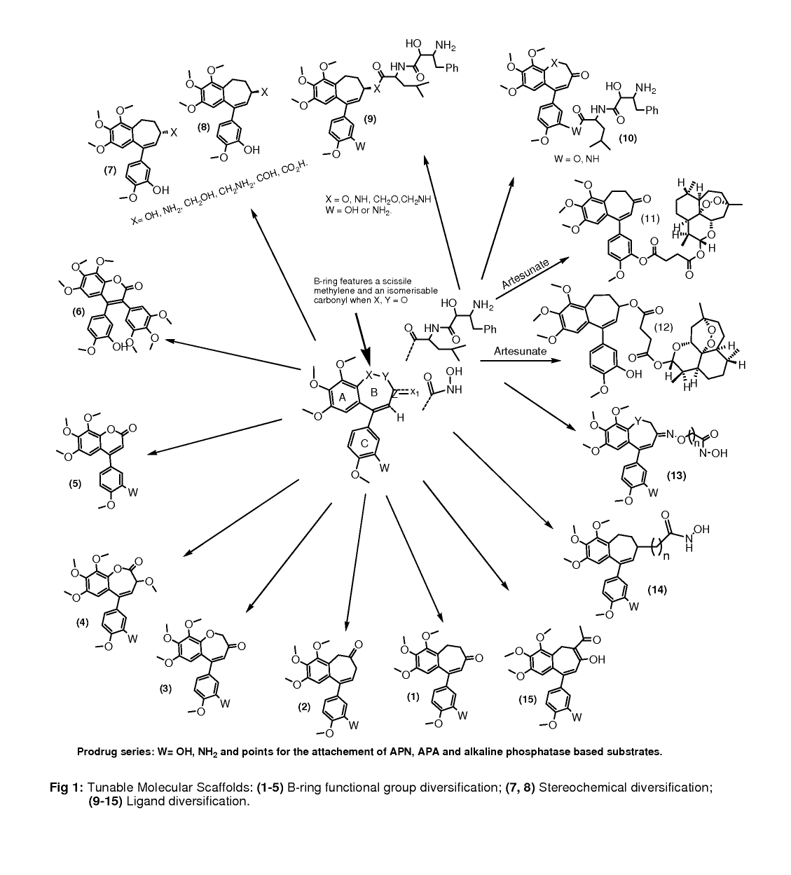

The design principally utilises the A and C-rings for single target specificity while the B-ring acts as the central axis for functional group, stereochemical, ligand diversification and enhancing activity of resultant compounds. The initial study focussed on precise functionalisation on the A- and C-rings and is limited to lower alkoxy-substituents and locations shown in the individual scaffolds for tubulin binding, FIG. 1. In particular, generation of compounds capable of potent inhibition of tubulin polymerisation necessitated inclusion of either carbonyl or hydroxyl groups on the B-ring. Typically the synthesis commenced from 2,3,4-trimethoxybenzaldehyde and in situations where X=CH₂ or O, their synthesis converges after the preparation of their respective aryl-C3 acid side chains. In examples where X=O, their synthesis generally requires three steps to furnish the intermediate acid aryl-C3 acid. It involves Baeyer Villager oxidation of the 2,3,4-trimethoxybenzaldehyde followed by base hydrolysis to give a phenol. Alkylation of the phenol with methyl or ethyl bromoacetate yields an ester intermediate which is hydrolysed to the corresponding acid. Where X=CH₂, 2,3,4-trimethoxybenzaldehyde is treated with a solution of malonic acid in pyridine/piperidine to give an arylpropenoic acid intermediate which is reduced to the corresponding propanoic acid derivative. Coupling with Meldrum's acid followed by methanolysis gives their respective β-keto esters, which following; their stereoselective reduction with Baker's yeast or non-stereoselective reduction with sodium borohydride, protection with t-BDMSC1 and hydrolysis affords acid precursors to the A-B ring. Following acid chloride formation and their respective cyclisation with SnCl₄, the A-B rings are formed. The isomeric A-B ring within scaffold 2, FIG. 1, where Y is functionalised is furnished following reduction of 3,4,5-trimethoxybenzaldehyde with sodium borohydride, hydroxy substitution with PBr₃, followed by cyanide displacement and allylic insertion under modified Barbier conditions to give an allylic ketone. Ketone reduction of said intermediate and protection of the resultant alcohol with TBDPSCl is followed by a borane reaction and oxidation to afford the acid precursor to the A-B ring. Cyclisation of said acid affords the A-B ring intermediate. Attachment of the meta-hydroxy based C-rings can be accomplished through either forming the triflate intermediate of the A-B rings and then attaching the C-ring under Suzuki conditions or following removal of the t-BDPS group from these rings with TBAF and coupling of the C-ring using an organolithium reaction will afford the A-B-C ring structure while oxidation of their B-ring alcohols to carbonyl-containing compounds can be conducted with PDC, PCC or Dess-Martin periodinane. Removal of the t-BDMS group from their C rings can be accomplished with TBAF in THF or NaN₃ in DMF. Attachment of meta amino based C-rings to the A-B ring intermediates was accomplished by coupling N—BOC protected boronic acids to triflate derivatives of the A-B rings. Generation of the “nature series” of 4-phenyl chromen-2-ones, utilised a novel ring contraction reaction, involving initial site-specific insertion of bromide, using phenyltrimethylammonium tribromide, and then displacement by azide, to yield an azo-oxymethylene enone intermediate, which following gaseous expulsion, gives the ring contracted 4-phenyl-chromen-2-one series (scaffold 5, FIG. 1). Moving the “carbonyl” from position Z to Y involved treatment of the same intermediate with methanol to afford the isomerised 3-methoxy series of ligands (scaffold 6, FIG. 1). In the case of scaffold 2, FIG. 1, the central reaction involves utilisation of Barbier type conditions to generate an allylic ketone intermediate from 2,3,4-trimethoxybenzonitrile. The vinylic proton on the chromen-2-one scaffold (5) was exploited to give the designed multiple ligand series following bromination with pyridinium tribromide and attachment of the D-ring under Suzuki conditions. The single enantiomers within scaffolds 7 and 8 required devising appropriate syntheses of the individual 7R and S forms as exemplified within these scaffolds. Central to the synthetic accomplishment of this series of stereoisomers was utilisation of Baker's yeast to facilitate the synthesis of an enantiomerically pure S-alcohol by reduction of the β-keto ester intermediate. The corresponding R-isomers were generally furnished from enantiomercially pure A-B ring intermediates. For example, the corresponding R-alcohol at the Z-position required the carbonyl group of the A-B ring intermediates to be reduced and protected as a MOM derivative. Then the silicon-based protecting group was released at the Z position and following mesylation and treatment with caesium acetate the opposite stereoisomer at this centre was afforded after the acetate hydrolysis under basic conditions. Introduction of a one-carbon spacer group between the amino/hydroxy substituent on the Z position of series 7 and 8 was facilitated by cyanide displacement of the same mesylates as above with the key steps employing a variety of reducing agents; LiAlH₄ to furnish the amines and a double reduction step employing DIBAL and sodium borohydride to furnish the alcohols. Engineering the APN binding component into the Z-position on the B-ring required a sequential approach with the ester linked hybrids (scaffold 9, FIG. 1). Firstly, N-FMOC-leucine can be coupled independently to the R- and S-alcohols, and following N-FMOC deprotection with TBAF, coupling of the second amino acid, N-FMOC-[(2S,3R)-3-amino-2-hydroxy-4-phenylbutanoic acid-pentafluorophenyl ester is effected. Finally liberation of the FMOC group is carried out using TBAF in THF or piperidine in DMF. The amide series within this scaffold required coupling of N—BOC-[(2S,3R)-3-Amino-2-hydroxy-4-phenylbutyryl]-L-leucine to the amino enantiomers using PyBrOP as coupling reagent. Presentation of the tubulin binding component in prodrug form, including examples that also utilise an APN inhibitor on the C-ring involved coupling of N—BOC-leucine to the phenolic or aniline based C-rings, subsequent N—BOC deprotection using anhydrous trifluoroacetic acid in DCM and in the case of the hybrid forms presented in scaffold 12, FIG. 1, a further coupling step was employed using the pentafluorophenylester of N—BOC—N-(2S,3R)-3-amino-2-hydroxy-4-phenylbutanoic acid which was followed by N—BOC deprotection. Extension of this technology to other drugs with anti-angiogenic activity was demonstrated with artensunate (scaffolds 11 and 12, FIG. 1). The designed multiple ligand series of scaffolds, as exemplified by scaffold 13 involves exploitation of the carbonyl group within scaffolds 1-6 (FIG. 1) to append the APN targeting hydroxamic acid moiety via an oximino methyl spacer group. The designed multiple ligand series of hydroxamic acids devoid of a oximino spacer group were furnished following oxidation of the primary alcohols within scaffolds 7 and 8 (FIG. 1) on the B-ring to aldehydes, with Dess-Martin periodinane, and subsequent oxidation with sodium chlorite or pyridinium chlorochromate. Activation of the resultant acids as pentafluorophenyl esters and their displacement with hydroxylamine hydrochloride neatly afforded the hydroxamic acid series. The β-hydroxy ketone series (scaffold 15, FIG. 1) can be generated following acylation alpha to the carbonyl group within scaffolds 1 and 3 (FIG. 1) using pyruvyl nitrile as acylation source and lithium diisopropylamide as base. Note: In scaffolds 9, 13, 14 and 15 of FIG. 1, W is OH or NH₂.

### EXPERIMENTAL

### Synthesis

### Experimental

### Examples

### Preparation of (Z)-8,9-dihydro-5-(3-hydroxy-4-methoxyphenyl)-1,2,3-trimethoxybenzo[7]annulen-7-one 1

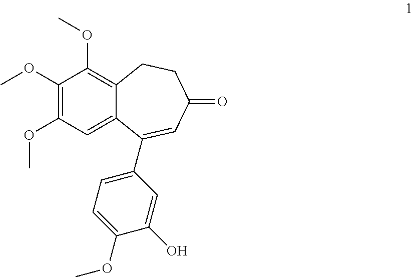 [SMILES: COc1ccc(C2=CC(=O)CCc3c2cc(OC)c(OC)c3OC)cc1O]

### Step 1

### Synthesis of (E)-3-(2,3,4-trimethoxyphenyl)acrylic acid 1.2

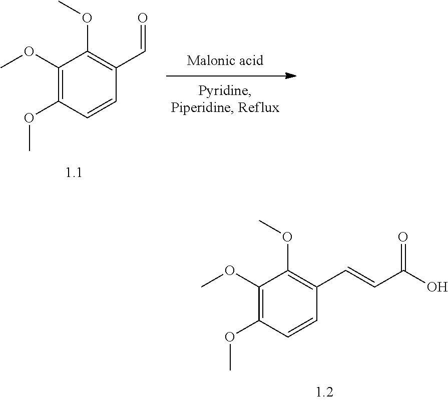 [SMILES: COc1ccc(C=O)c(OC)c1OC ; COc1ccc(/C=C/C(=O)O)c(OC)c1OC]

To a stirred solution of 2,3,4-trimethoxybenzaldehyde 1.1 (10.00 g, 51.00 mmol) in a mixture of pyridine (30 mL) and piperidine (0.6 mL) was added malonic acid (10.60 g, 102.10 mmol).

The mixture was refluxed for 6 h. The reaction was quenched by the addition of 2M aq. HCl (1×100 mL) and extracted with ethyl acetate (3×60 mL). The solvent was removed in vacuo to afford (E)-3-(2,3,4-trimethoxyphenyl)acrylic acid 1.2 as an off-white solid (11.98 g, 99%). The product was not further purified.

¹H NMR (CDCl₃, 400 MHz) δ_{H} ppm: 3.90 (3H, s, OMe, 3.93 (3H, s, OMe, 3.96 (3H, s, OMe, 6.45 (1H, d, J=16.0 Hz, CH═CH), 6.73 (1H, d, J=8.5 Hz, ArH), 7.32 (1H, d, J=8.5 Hz, ArH), 8.01 (1H, d, J=16.0 Hz, CH═CH)

¹³C NMR (CDCl₃, 400 MHz) δ_{c} ppm: 55.63 (OMe, 60.47 (OMe, 61.04 (OMe, 107.15 (ArCH), 115.60 (CH═CH), 120.68 (ArC), 123.13 (ArCH), 141.56 (CH═CH), 141.88 (ArC), 153.08 (ArC), 155.48 (ArC), 172.49 (C═O)

νₘₐₓ (KBr)/cm⁻¹ 3452.6, 2944.1, 1694.2, 1619.4, 1590.0

Melting point: 160-162° C.

### Step 2

### Synthesis of 3-(2,3,4-trimethoxyphenyl)propanoic acid 1.3

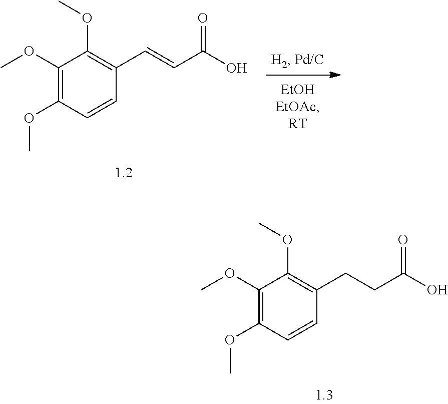 [SMILES: COc1ccc(/C=C/C(=O)O)c(OC)c1OC ; COc1ccc(CCC(=O)O)c(OC)c1OC]

To a stirred solution of (E)-3-(2,3,4-trimethoxyphenyl)acrylic acid 1.2 (500 mg, 2.11 mmol) in a 1:1 mixture of ethanol and ethyl acetate (10 mL) was added at catalytic amount of 10% Pd/C under an atmosphere of hydrogen gas (balloon). After 18 h the reaction mixture was filtered and the solvent was removed in vacuo to afford an off-white solid. The resulting residue redissolved in diethyl ether (20 mL) and was washed with 2.5M aq. NaOH (3×20 mL). The combined aqueous extracts were acidified with 2M aq. HCl and the product was extracted with diethyl ether (3×30 mL). The combined ether extracts were dried over MgSO₄, filtered and concentrated in vacuo to afford 3-(2,3,4-trimethoxyphenyl)propanoic acid 1.3 as a white solid (500 mg, 100%).

¹H NMR (CDCl₃, 400 MHz) δ_{H} ppm: 2.66 (2H, t, J=7.5 Hz, CH₂), 2.90 (2H, t, J=7.5 Hz, CH₂), 3.86 (3H, s, OMe), 3.88 (3H, s, OMe), 3.92 (3H, s, OMe), 6.61 (1H, d, J=8.0 Hz, ArH), 6.87 (1H, d, J=8.0 Hz, ArH)

¹³C NMR (CDCl₃, 400 MHz) δ_{c} ppm: 24.72 (CH₂), 34.45 (CH₂), 55.53 (OMe), 60.27 (OMe), 60.41 (OMe), 106.62 (ArCH), 123.33 (ArCH), 125.60 (ArC), 141.74 (ArC), 151.44 (ArC), 152.06 (ArC), 191.57 (C═O)

νₘₐₓ (KBr)/cm⁻¹ 3004.2, 2834.9, 1712.9, 1599.5

HRMS: calculated 263.0895, found 263.0845, molecular formula (C₁₂H₁₆O₅Na).

Melting point: 65-67° C.

### Step 3

### Synthesis of methyl 5-(2,3,4-trimethoxyphenyl)-3-oxopentanoate 1.5

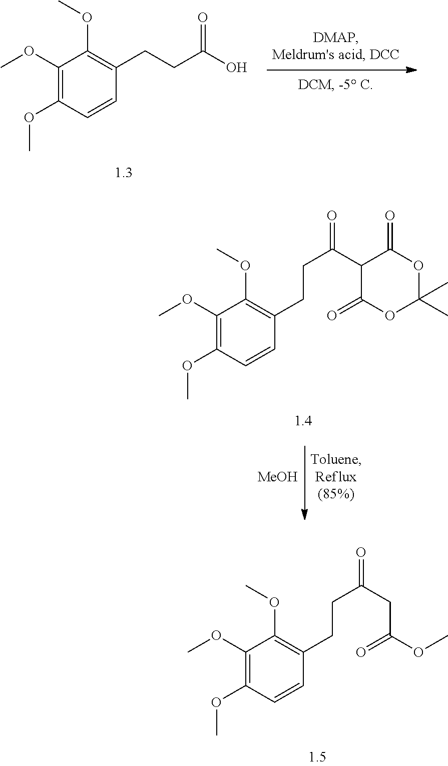

To a stirred solution of 3-(2,3,4-trimethoxyphenyl)propanoic acid 1.3 (2.00 g, 8.31 mmol) in anhydrous DCM (40 mL) was added DMAP (2.00 g, 16.70 mmol) followed by Meldrum's acid (2.41 g, 16.72 mmol) at room temperature under anhydrous conditions. DCC (3.52 g, 16.72 mmol) dissolved in dry DCM (10 mL) was added drop-wise to the reaction mixture at −5° C. The reaction was left stirring at this temperature for 90 min, after which time the flask was removed from the ice and allowed to increase to room temperature. The reaction was stirred at room temperature for a further 3 h. The precipitated dicyclohexyl urea was filtered from the mixture using DCM. The DCM extract was then washed with 2M aq. HCl (2×50 mL) and water (1×50 mL), dried over MgSO₄, filtered and concentrated in vacuo. The resulting residue, a viscous yellow oil, was dissolved in a 4:1 mixture of toluene and methanol, respectively, (36 mL) and was refluxed for 3 h. The solvent was removed from the flask in vacuo. The resulting residue was purified by flash column chromatography (stationary phase; silica gel 230-400 mesh, mobile phase; 4:1, hexane/ethyl acetate). All homogenous fractions were collected and the solvent was evaporated to afford methyl 5-(2,3,4-trimethoxyphenyl)-3-oxopentanoate 1.5 as a yellow oil (2.11 g, 85%).

¹H NMR (CDCl₃, 400 MHz) δ_{H} ppm: 2.82 (4H, s, ArCH₂CH₂), 3.44 (2H, S, COCH₂CO), 3.71 (3H, s, COOCH₃), 3.82 (3H, s, OMe), 3.85 (3H, s, OMe), 3.87 (3H, s, OMe), 6.57 (1H, d, J=8.5 Hz, ArH), 6.81 (1H, d, J=8.5 Hz, ArH)

¹³C NMR (CDCl₃, 400 MHz) δ_{c} ppm: 24.08 (CH₂), 43.77 (CH₂), 48.99 (CH₂), 52.24 (COOMe), 55.88 (OMe), 60.63 (OMe), 60.76 (OMe), 107.07 (ArCH), 123.86 (ArCH), 125.81 (ArC), 141.74 (ArC), 151.33 (ArC), 151.95 (ArC), 167.16 (C═O), 201.79 (C═O)

νₘₐₓ (DCM)/cm⁻¹ 2940.1, 1748.1, 1716.7, 1602.6, 1495.6, 1467.3, 1097.7

HRMS: calculated 319.1158, found 319.1155, molecular formula (C₁₅H₂₀O₆Na).

### Step 4

### Synthesis of methyl 3-hydroxy-5-(2,3,4-trimethoxyphenyl)pentanoate 1.6

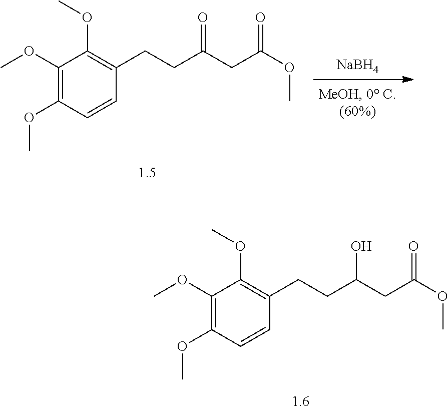

To a stirred solution of methyl 5-(2,3,4-trimethoxyphenyl)-3-oxopentanoate 1.5 (2.00 g, 6.75 mmol) in methanol (30 mL) was added NaBH₄ (0.09 g, 2.25 mmol) at 0° C. The reaction was allowed to stir at this temperature for 1 h. It was then removed from the ice and allowed to increase to room temperature. The progress of the reaction was monitored by TLC and after a total 3 h the reaction was quenched by the addition of water (1×50 mL). The mixture was heated under vacuum to remove the methanol and the product was extracted with diethyl ether (3×30 mL). The organic fractions were combined, dried over MgSO₄, filtered and concentrated in vacuo. The resulting residue was purified by flash column chromatography (stationary phase; silica gel 230-400 mesh, mobile phase; 2:1, hexane/ethyl acetate). All homogenous fractions were collected and the solvent was evaporated to afford methyl 3-hydroxy-5-(2,3,4-trimethoxyphenyl)pentanoate 1.6 as a clear, colourless oil (1.20 g, 60%).

¹H NMR (CDCl₃, 400 MHz) δ_{H} ppm: 1.71 (2H, m, CH₂), 2.51 (2H, m, CH₂), 2.71 (2H, m, CH₂), 3.72 (3H, s, COOCH₃), 3.86 (3H, s, OMe), 3.87 (3H, s, OMe), 3.90 (3H, s, OMe), 4.02 (1H, m, CHOH), 6.63 (1H, d, J=8.5 Hz, ArH), 6.86 (1H, d, J=8.5 Hz, ArH)

¹³C NMR (CDCl₃, 400 MHz) δ_{c} ppm: 25.14 (CH₂), 37.19 (CH₂), 40.72 (CH₂), 51.29 (COOMe), 55.55 (OMe), 60.33 (OMe), 60.56 (OMe), 66.74 (CHOH), 106.94 (ArCH), 123.53 (ArCH), 126.91 (2×ArC), 151.32 (ArC), 151.65 (ArC), 172.83 (C═O)

νₘₐₓ (DCM)/cm⁻¹ 3489.9, 2938.6, 1735.3, 1601.9, 1495.4, 1466.4

HRMS: calculated 321.1314, found 321.1301, molecular formula (C₁₅H₂₂O₆Na).

### Step 5

### Synthesis of Methyl 3-tert-butyl-diphenylsilyloxy-5-(2,3,4-trimethoxyphenyl)pentanoate 1.7

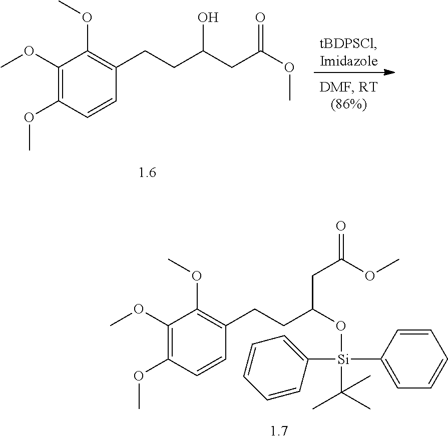

Methyl 3-hydroxy-5-(2,3,4-trimethoxyphenyl)pentanoate 1.6 (3.20 g, 10.80 mmol) was dried in vacuo for 24 h, prior to being dissolved in DMF (20 mL). Tert-butyl-diphenylsilylchloride (4.2 mL, 16.20 mmol) and imidazole (1.20 g, 17.30 mmol) were added to the stirred solution at room temperature under an atmosphere of nitrogen. The reaction was left stirring over night. It was quenched by the addition of sat. aq. NaCl (1×50 mL) and the protected alcohol was extracted with diethyl ether (3×30 mL). The organic fractions were combined, dried over MgSO₄, filtered and concentrated in vacuo. The resulting residue was purified by flash column chromatography (stationary phase; silica gel 230-400 mesh, mobile phase; 9:1, hexane/ethyl acetate). All homogenous fractions were collected and the solvent was evaporated to afford Methyl 3-tert-butyl-diphenylsilyloxy-5-(2,3,4-trimethoxyphenyl)pentanoate 1.7 as a yellow oil (5.00 g, 86%).

¹H NMR (CDCl₃, 400 MHz) δ_{H} ppm: 1.08 (9H, s, C(CH₃)₃), 1.76 (2H, m, CH₂), 2.58 (4H, m, 2×CH₂), 3.57 (3H, s, COOCH₃), 3.78 (3H, s, OMe), 3.85 (3H, s, OMe), 3.86 (3H, s, OMe), 4.30 (1H, qn, J=6.0 Hz, CHOH), 6.55 (1H, d, J=8.5 Hz, ArH), 6.61 (1H, d, J=8.5 Hz, ArH), 7.39 (6H, m, ArH), 7.72 (4H, m, ArH)

¹³C NMR (CDCl₃, 400 MHz) δ_{c} ppm: 18.91 (C(CH₃)₃), 25.00 (CH₂), 26.90 (C(CH₃)₃), 38.20 (CH₂), 41.75 (CH₂), 51.38 (COOMe), 55.94 (OMe), 60.65 (OMe), 60.74 (OMe), 70.28 (CHOH), 107.06 (ArCH), 123.45 (ArCH), 127.08 (ArC), 127.48 (4×ArCH), 129.55 (2×ArCH), 133.54 (ArC), 135.88 (4×ArCH), 141.71 (ArC), 151.28 (ArC), 151.44 (ArC), 171.46 (C═O)

νₘₐₓ (DCM)/cm⁻¹ 2933.4, 1739.9, 1602.7, 1494.9, 1466.9, 1104.1

HRMS: calculated 559.2492, found 559.2488, molecular formula (C₃₁H₄₀O₆NaSi).

### Step 6

### Synthesis of 3-tert-butyl-diphenylsilyloxy-5-(2,3,4-trimethoxyphenyl)pentanoic acid 1.8

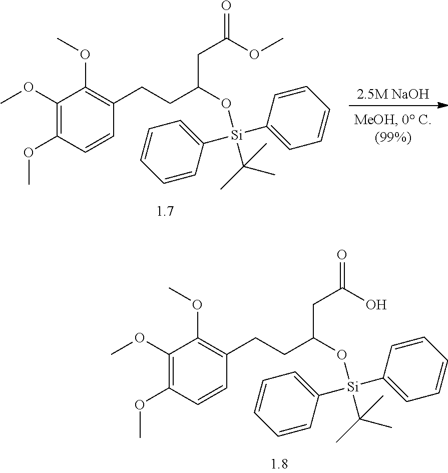 [SMILES: COC(=O)CC(CCc1ccc(OC)c(OC)c1OC)O[Si](c1ccccc1)(c1ccccc1)C(C)(C)C ; "2.5M NaOH" ; COc1ccc(CCC(CC(=O)O)O[Si](c2ccccc2)(c2ccccc2)C(C)(C)C)c(OC)c1OC]

To a stirred solution of methyl 3-tert-butyl-diphenylsilyloxy-5-(2,3,4-trimethoxyphenyl)pentanoate 1.7 (16.00 g, 29.90 mmol) in a mixture of methanol (50 mL) and THF (25 mL) was added 2.5M aq. NaOH (40 mL) at 0° C. The reaction was left stirring for 1 h after which time the flask was removed from the ice and was allowed to increase to room temperature. The reaction proceeded for an additional 13 h. The mixture was acidified with 2M aq. HCl (1×60 mL). The organic solvents were removed from the mixture by heating under vacuum. The acid was extracted with diethyl ether (3×150 mL). The organic fractions were combined, dried over MgSO₄, filtered and concentrated in vacuo. The resulting residue was purified by flash column chromatography (stationary phase; silica gel 230-400 mesh, mobile phase; 4:1, hexane/ethyl acetate). All homogenous fractions were collected and the solvent was evaporated to afford 3-tert-butyl-diphenylsilyloxy-5-(2,3,4-trimethoxyphenyl)pentanoic acid 1.8 as a white solid (15.50 g, 99%).

¹H NMR (CDCl₃, 400 MHz) δ_{H} ppm: 1.08 (9H, s, C(CH₃)₃), 1.76 (2H, m, CH₂), 2.60 (4H, m, 2×CH₂), 3.77 (3H, s, OMe), 3.85 (6H, s, 2×OMe), 4.24 (1H, m, CHOH), 6.53 (1H, d, J=8.5 Hz, ArH), 6.60 (1H, d, J=8.5 Hz, ArH), 7.42 (6H, m, ArH), 7.72 (4H, m, ArH)

¹³C NMR (CDCl₃, 400 MHz) δ_{c} ppm: 18.87 (C(CH₃)₃), 25.09 (CH₂), 26.50 (C(CH₃)₃), 37.92 (CH₂), 41.30 (CH₂), 55.94 (OMe), 60.64 (OMe), 60.73 (OMe), 70.12 (CHOH), 107.07 (ArCH), 123.44 (ArCH), 127.10 (ArC), 127.20 (4×ArCH), 129.55 (2×ArCH), 133.54 (ArC), 135.88 (4×ArCH), 141.71 (ArC), 151.28 (ArC), 151.44 (ArC), 171.46 (C═O)

νₘₐₓ (KBr)/cm⁻¹ 3049.1, 2930.4, 1709.6, 1604.8, 1495.9, 1465.1

HRMS: calculated 545.2335, found 545.2350, molecular formula (C₃₀HO₆NaSi).

Melting point: 110-112° C.

### Step 7

### Synthesis of 7-tert-butyl-diphenyl-silyloxy-6,7,8,9-tetrahydro-1,2,3-trimethoxybenzo[7]annulen-5-one 1.9

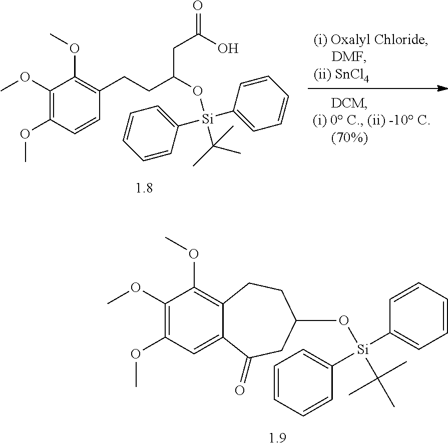 [SMILES: COc1ccc(CCC(CC(=O)O)O[Si](c2ccccc2)(c2ccccc2)C(C)(C)C)c(OC)c1OC ; "(i) Oxalyl Chloride,     DMF,(ii) SnCl4" ; COc1cc2c(c(OC)c1OC)CCC(O[Si](c1ccccc1)(c1ccccc1)C(C)(C)C)CC2=O]

To a stirred solution of 3-tert-butyl-diphenylsilyloxy-5-(2,3,4-trimethoxyphenyl)pentanoic acid 1.8 (500 mg, 0.96 mmol) in anhydrous DCM (4 mL) was added oxalyl chloride (0.4 mL, 4.80 mmol) and DMF (1 drop) at 0° C. After 2 h, the excess oxalyl chloride was removed under reduced pressure to afford the corresponding acid chloride as a brown viscous oil. This oil was dissolved in anhydrous DCM (10 mL) and 1M SnCl₄ (0.32 mL, 0.32 mmol) was added at −10° C. After 30 min the reaction was quenched with sat. aq. NaCl (1×10 mL) and the product was extracted using diethyl ether (4×20 mL). The organic fractions were collected, dried over MgSO₄ and filtered. The solvent was removed in vacuo to afford a viscous yellow oil/foam. The product was purified by flash column chromatography (stationary phase; silica gel 230-400 mesh, mobile phase; 4:1, hexane/ethyl acetate). All homogenous fractions were collected and the solvent was evaporated to afford 7-tert-butyl-diphenyl-silyloxy-6,7,8,9-tetrahydro-1,2,3-trimethoxybenzo[7]annulen-5-one 1.9 as a clear colourless oil (340 mg, 70%).

¹H NMR (CDCl₃, 400 MHz) δ_{H} ppm: 1.08 (9H, s, C(CH₃)₃), 1.96 (2H, m, CH₂), 2.95 (2H, m, CH₂), 3.13 (2H, m, CH₂), 3.85 (3H, s, OMe), 3.88 (3H, s, OMe), 3.95 (3H, s, OMe), 4.36 (1H, m, CHOP), 7.21 (1H, s, ArH), 7.41 (6H, m, ArH), 7.72 (4H, m, ArH)

¹³C NMR (CDCl₃, 400 MHz) δ_{c} ppm: 18.71 (C(CH₃)₃), 20.54 (CH₂), 26.85 (C(CH₃)₃), 36.32 (CH₂), 50.21 (CH₂), 55.89 (OMe), 60.80 (OMe), 61.08 (OMe), 68.22 (CHOP), 107.47 (ArCH), 127.25 (4×ArCH), 129.36 (2×ArCH), 130.40 (ArC), 133.28 (ArC), 133.46 (ArC), 134.21 (ArC), 135.34 (4×ArCH), 145.11 (ArC), 150.65 (ArC), 150.97 (ArC), 199.16 (C═O)

νₘₐₓ (DCM)/cm⁻¹ 3495.7, 2933.8, 1674.2

HRMS: calculated 527.2230, found 527.2222, molecular formula (C₃₀H₃₆O₅NaSi).

### Step 8

### (E)-7-tert-butyl-diphenylsilyloxy-6,7-dihydro-2,3,4-trimethoxy-5H-benzo[7]annulen-9-yl trifluoromethanesulfonate 1.11

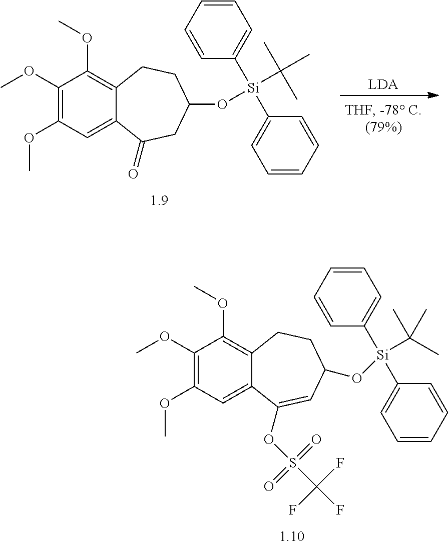 [SMILES: COc1cc2c(c(OC)c1OC)CCC(O[Si](c1ccccc1)(c1ccccc1)C(C)(C)C)CC2=O ; "LDA" ; COc1cc2c(c(OC)c1OC)CCC(O[Si](c1ccccc1)(c1ccccc1)C(C)(C)C)C=C2OS(=O)(=O)C(F)(F)F]

To a dry three-necked round bottom flask containing N,N-diisopropylamine (0.05 mL, 0.33 mmol) was added 2.5M nBuLi (0.13 mL, 0.33 mmol) under dry reaction conditions at −78° C. After 20 min a solution of 7-tert-butyl-diphenyl-silyloxy-6,7,8,9-tetrahydro-1,2,3-trimethoxybenzo[7]annulen-5-one 1.9 (150 mg, 0.30 mmol) in dry THF (2 mL) was transferred to the three-necked flask, drop-wise via a syringe. The resultant suspension was allowed to stir at −78° C. for 2 h and a solution of 2-[N,N-bis(trifluoromethylsulfonyl)amino]-5-chloropyridine (130 mg, 0.33 mmol) in dry THF (2 mL) was added. The reaction was allowed to stir for an additional 3 h at this temperature. The reaction was quenched by the addition of water (1×50 mL) and extracted with diethyl ether (3×50 mL). The combined organic fractions were dried over MgSO₄, filtered and dried under vacuum. The residue was purified by flash column chromatography (stationary phase; silica gel 230-400 mesh, mobile phase; 8:1, hexane/ethyl acetate) to yield (E)-7-tert-butyl-diphenylsilyloxy-6,7-dihydro-2,3,4-trimethoxy-5H-benzo[7]annulen-9-yl trifluoromethanesulfonate 1.10 as a colourless oil (160 mg, 79%).

¹H NMR (CDCl₃, 400 MHz) δ_{H} ppm: 1.10 (9H, s, C(CH₃)₃), 1.91 (1H, m, CH₂), 2.30 (1H, m, CH₂), 2.57 (1H, m, CH₂), 2.99 (1H, m, CH₂), 3.75 (3H, s, OMe), 3.86 (3H, s, OMe), 3.92 (3H, s, OMe), 4.30 (1H, m, CHOP), 6.13 (1H, d, J=4.5 Hz, C═CH), 6.81 (1H, s, ArH), 7.38-7.68 (10H, m, 10×ArH)

¹³C NMR (CDCl₃, 400 MHz) δ_{c} ppm: 18.57 (C(CH₃)₃), 20.43 (CH₂), 26.88 (C(CH₃)₃), 39.75 (CH₂), 55.96 (OMe), 60.95 (OMe), 61.53 (OMe), 68.60 (CHOP), 105.91 (ArCH), 126.09 (C═CH), 126.54 (ArC), 127.22 (2×ArCH), 127.28 (ArC), 127.31 (ArCH), 128.51 (ArCH), 129.21 (ArC), 129.44 (2×ArCH), 132.92 (ArC), 133.10 (2×ArCH), 143.10 (ArC), 143.91 (ArC), 151.09 (ArC), 151.44 (ArC)

¹⁹F NMR (CDCl₃, 400 MHz) δ_{F} ppm: −74.49

νₘₐₓ (DCM)/cm⁻¹ 3467.3, 2932.3, 1595.0, 1419.8, 1211.8, 1113.4

### Step 9

### Synthesis of 5-bromo-2-methoxyphenol 1.13

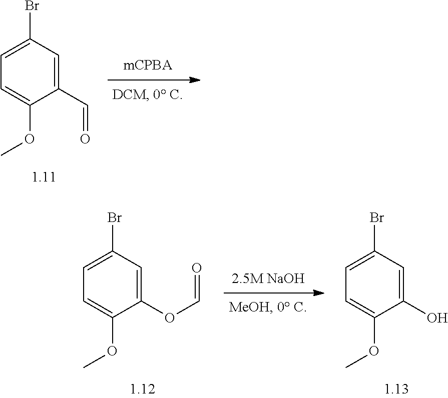

To a stirred solution of 5-bromo-2-methoxy-benzaldehyde 1.11 (5.00 g, 23.30 mmol) in DCM (25 mL) was added a solution of mCPBA (4.80 g, 28.00 mmol) dissolved in DCM (40 mL). After 5 h, the mixture was filtered to remove the precipitated m-chlorobenzoic acid. The filtrate was washed with 5% aq. NaHCO₃ (2×50 mL), water (1×50 mL) and sat. aq. NaCl (1×50 mL). The organic layer was then washed with 2.5M aq. NaOH (2×50 mL); the aqueous layer was acidified with 2M aq. HCl and extracted with DCM (2×50 mL). The organic fractions were collected, dried over MgSO₄ and filtered. The solvent was removed in vacuo to afford 5-bromo-2-methoxyphenol 1.13 as a yellow solid (3.70 g, 79%).

¹H NMR (CDCl₃, 400 MHz) δ_{H} ppm: 3.80 (3H, s, OMe), 5.61 (1H, s, OH), 6.64 (1H, d, J=8.5 Hz, ArH), 6.89 (1H, dd, J=2.5 Hz, 8.5 Hz, ArH), 7.00 (1H, d, J=2.0 Hz, ArH)

¹³C NMR (CDCl₃, 400 MHz) δ_{c} ppm: 55.63 (OMe), 111.37 (ArCH), 113.00 (ArC), 117.36 (ArCH), 122.34 (ArCH), 145.59 (ArC), 146.02 (ArC)

νₘₐₓ (KBr)/cm⁻¹ 3399.6, 1592.6, 621.3

Melting point: 60-62° C.

### Step 10

### Synthesis of (5-bromo-2-methoxyphenoxy)(tert-butyl)dimethylsilane 1.14

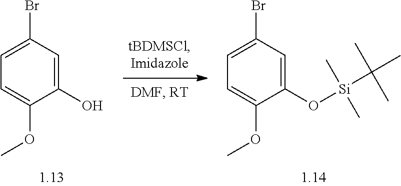

To a stirred solution of 5-bromo-2-methoxyphenol 1.13 (2.30 g, 11.33 mmol) in DMF (15 mL) was added imidazole (1.90 g, 28.30 mmol) and tert-butyl-dimethylsilylchloride (3.40 g, 15.30 mmol) at room temperature, under anhydrous conditions. After 4 h the reaction was quenched by the addition of water (1×25 mL) and the crude product was extracted with diethyl ether (3×20 mL). The combined organic extracts were dried over MgSO₄, filtered and the solvent was removed under reduced pressure to afford a pale yellow oil. The product was purified by flash column chromatography (stationary phase; silica gel 230-400 mesh, mobile phase; 9:1, hexane/ethyl acetate). All homogenous fractions were collected and the solvent was evaporated to afford (5-bromo-2-methoxyphenoxy)(tert-butyl)dimethylsilane 1.14 as a clear, colourless oil (3.30 g, 100%).

¹H NMR (CDCl₃, 400 MHz) δ_{H} ppm: 0.21 (6H, s, 2×Si(CH₃)), 1.05 (9H, s, C(CH₃)₃), 3.80 (3H, s, OMe), 6.73 (1H, d, J=8.5 Hz, ArH), 7.04 (1H, m, ArH), 7.06 (1H, d, J=2.5 Hz, ArH)

¹³C NMR (CDCl₃, 400 MHz) δ_{c} ppm: −5.08 (2×Si(CH₃)), 18.01 (C(CH₃)₃), 25.25 (C(CH₃)₃), 55.08 (OMe), 111.92 (ArC), 112.73 (ArCH), 123.62 (ArCH), 124.00 (ArCH), 145.52 (ArC), 149.98 (ArC)

νₘₐₓ (DCM)/cm⁻¹ 2930.5, 1585.8, 1497.1, 1269.6, 934.9, 623.0

### Step 11

### Preparation of 3-tert-butyl-dimethylsilyloxy-4-methoxyphenylboronic acid 1.15

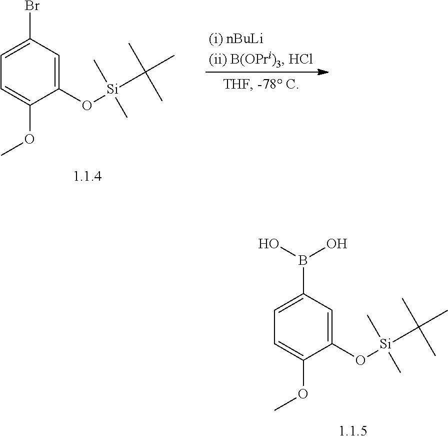 [SMILES: COc1ccc(Br)cc1O[Si](C)(C)C(C)(C)C ; COc1ccc(B(O)O)cc1O[Si](C)(C)C(C)(C)C]

To a stirred solution of (5-bromo-2-methoxyphenoxy)(tert-butyl)dimethylsilane 1.14 (2.22 g, 7.03 mmol) dissolved in anhydrous THF (4 mL) was added 2.5M nBuLi (4.50 mL, 11.25 mmol) drop-wise at −78° C., under anhydrous conditions. After 20 min, whilst maintaining the temperature at −78° C., triisopropyl borate (8.44 mL, 36.56 mmol) was added drop-wise to the reaction. After 2 h the temperature was allowed to increase to −20° C. and after an additional 2 h the temperature was allowed to increase to ambient. The reaction was maintained at this temperature for 24 h. The reaction was quenched by the addition of 2M aq. HCl (1×100 mL). The product was extracted with diethyl ether (3×100 mL). The organic fractions were combined, dried over MgSO₄, filtered and concentrated in vacuo. The resulting residue was purified by flash column chromatography (stationary phase; silica gel 230-400 mesh, mobile phase; 9:1, ethyl acetate/methanol). All homogenous fractions were collected and the solvent was evaporated to afford the 3-tert-butyl-dimethylsilyloxy-4-methoxyphenylboronic acid 1.15 as a white solid (1.3 g, 66%).

¹H NMR (CDCl₃, 400 MHz) δ_{H} ppm: 0.25 (6H, s 2×Si(CH₃)), 1.09 (9H, s, C(CH₃)₃), 3.92 (3H, s, OMe), 7.01 (1H, d, J=8.0 Hz, ArH), 7.69 (1H, d, J=1.5 Hz, ArH), 7.84 (1H, dd, J=1.5 Hz, 8.0 Hz, ArH)

¹³C NMR (CDCl₃, 400 MHz) δ_{c} ppm: −4.96 (2×Si(CH₃)), 18.08 (C(CH₃)₃), 25.38 (C(CH₃)₃), 54.86 (OMe), 110.86 (ArCH), 126.95 (ArCH), 129.83 (ArCH), 144.07 (ArC), 154.33 (ArC)

νₘₐₓ (KBr)/cm⁻¹ 2930.3, 1599.1, 1511.9, 1411.9, 1318.6, 1269.11, 840.5

Melting point: 149-155° C.

### Step 12

### Synthesis of (5-((Z)-7-tert-butyl-diphenylsilyloxy-6,7-dihydro-2,3,4-trimethoxy-5H-benzo[7]annulen-9-yl)-2-methoxyphenoxy)(tert-butyl)dimethylsilane 1.16

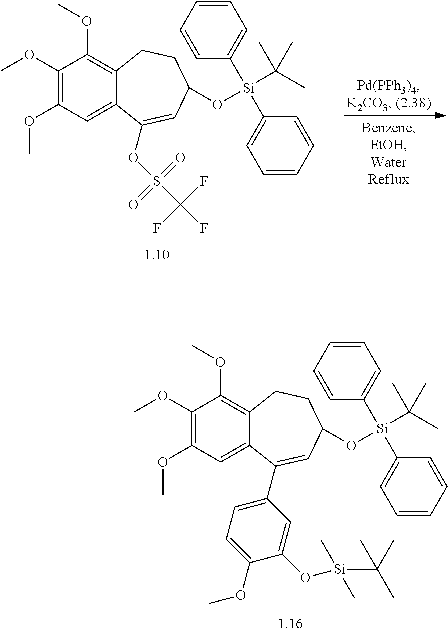 [SMILES: COc1cc2c(c(OC)c1OC)CCC(O[Si](c1ccccc1)(c1ccccc1)C(C)(C)C)C=C2OS(=O)(=O)C(F)(F)F ; COc1ccc(C2=CC(O[Si](c3ccccc3)(c3ccccc3)C(C)(C)C)CCc3c2cc(OC)c(OC)c3OC)cc1O[Si](C)(C)C(C)(C)C]

To a flask containing (E)-7-tert-butyl-diphenylsilyloxy-6,7-dihydro-2,3,4-trimethoxy-5H-benzo[7]annulen-9-yl trifluoromethanesulfonate 1.10 (80 mg, 0.13 mmol) was added 3-tert-butyl-dimethylsilyloxy-4-methoxyphenylboronic acid 1.15 (42 mg, 0.15 mmol), K₂CO₃ (54 mg, 0.39 mmol), and Pd(Ph₃)₄ (8 mg, 0.007 mmol). The mixture was dissolved in a mixture of toluene, ethanol and water (3:1:1, 5 mL). The resulting mixture was refluxed for 30 min. The reaction was quenched by the addition of water (1×20 mL) and the product was extracted with diethyl ether (3×30 mL). The organic fractions were combined, dried over MgSO₄, filtered and concentrated in vacuo. The resulting residue was purified by flash column chromatography (stationary phase; silica gel 230-400 mesh, mobile phase; 8:1, hexane/ethyl acetate). All homogenous fractions were collected and the solvent was evaporated to afford (5-((Z)-7-tert-butyl-diphenylsilyloxy-6,7-dihydro-2,3,4-trimethoxy-5H-benzo[7]annulen-9-yl)-2-methoxyphenoxy)(tert-butyl)dimethylsilane 1.16 as a yellow oil (120 g, 100%).

¹H NMR (CDCl₃, 400 MHz) δ_{H} ppm: 0.20 (3H, s, SiCH₃), 0.22 (3H, s, SiCH₃), 1.06 (9H, s, C(CH₃)₃), 1.13 (9H, s, C(CH₃)₃), 2.33 (3H, m, 2×CH₂), 2.96 (1H, m, 2×CH₂), 3.68 (3H, s, OMe), 3.79 (3H, s, OMe), 3.88 (3H, s, OMe), 3.92 (3H, s, OMe), 4.20 (1H, m, CHOH), 6.25 (1H, s, ArH {A-ring}), 6.31 (1H, d, J=5.0 Hz, C═CH), 6.70 (3H, m, ArH {C-ring}), 6.75 (6H, m, ArH), 7.36 (4H, m, ArH)

¹³C NMR (CDCl₃, 400 MHz) δ_{c} ppm: −4.99 (Si(CH₃)₂), 18.06 (C(CH₃)₃), 18.72 (C(CH₃)₃), 21.40 (CH₂), 25.34 (C(CH₃)₃), 26.53 (C(CH₃)₃), 43.42 (CH₂), 55.11 (OMe), 55.45 (OMe), 60.42 (OMe), 61.13 (OMe), 70.88 (CH), 108.19 (ArCH), 111.19 (ArCH), 120.29 (ArCH), 121.06 (ArCH), 127.02 (2×ArCH), 127.28 (2×ArCH), 127.52 (ArC), 129.09 (ArCH), 129.19 (ArCH), 132.14 (ArC), 133.83 (ArC), 133.97 (ArCH), 134.40 (ArC), 135.36 (2×ArCH), 135.61 (2×ArCH), 137.25 (ArC), 140.75 (ArC), 144.14 (ArC), 150.03 (ArC), 150.19 (ArC), 150.40 (ArC)

νₘₐₓ (DCM)/cm⁻¹ 3468.2, 2931.5, 1509.2, 1113.4

### Step 13

### Synthesis of (Z)-6,7-dihydro-9-(3-hydroxy-4-methoxyphenyl)-2,3,4-trimethoxy-5H-benzo[7]annulen-7-ol 1.17

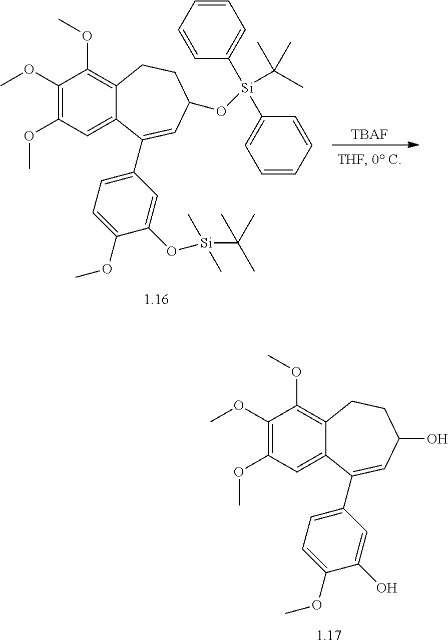

To a stirred solution of the (5-((Z)-7-tert-butyl-diphenylsilyloxy-6,7-dihydro-2,3,4-trimethoxy-5H-benzo[7]annulen-9-yl)-2-methoxyphenoxy)(tert-butyl)dimethylsilane 1.16 (210 mg, 0.29 mmol) in THF (2 mL) was added 1M TBAF (0.58 mL, 0.58 mmol) at 0° C. After 30 min the flask was removed from the ice and the temperature was allowed to increase to ambient. The reaction was allowed to stir for an additional 16 h. The reaction was then quenched by the addition of sat. aq. NaCl (1×10 mL) and the product was extracted with diethyl ether (3×20 mL). The ether extracts were combined, dried over MgSO₄ and filtered. The organic fractions were applied directly to a flash column, without prior concentration of the solution in vacuo. The product was purified by flash column chromatography (stationary phase; silica gel 230-400 mesh, mobile phase; 1:1, hexane/ethyl acetate). All homogenous fractions were collected and the solvent was evaporated to afford (Z)-6,7-dihydro-9-(3-hydroxy-4-methoxyphenyl)-2,3,4-trimethoxy-5H-benzo[7]annulen-7-ol 1.17 as a white solid (80 mg, 74%).

¹H NMR (CDCl₃, 400 MHz) δ_{H} ppm: 2.09 (1H, m, CH₂), 2.35 (1H, m, CH₂), 2.51 (1H, m, CH₂), 3.03 (1H, m, CH₂), 3.66 (3H, s, OMe), 3.87 (3H, s, OMe), 3.89 (6H, s, 2×OMe), 4.00 (1H, m, CHOH), 5.66 (1H, s, br, OH), 6.22 (1H, d, J=5.0 Hz, C═CH), 6.40 (1H, s, ArH {A-ring}), 6.74 (1H, dd, J=2.0 Hz, 8.5 Hz, ArH{C-ring}), 6.76 (1H, d, J=2.0 Hz, ArH{C-ring}), 6.88 (1H, d, J=8.0 Hz, ArH{C-ring})

¹³C NMR (CDCl₃, 400 MHz) δ_{c} ppm: 21.39 (CH₂), 42.28 (CH₂), 54.59 (OMe), 54.61 (OMe), 59.40 (OMe), 60.21 (OMe), 68.41 (CHOH), 108.78 (ArCH), 111.09 (ArCH), 114.59 (ArCH), 119.14 (ArCH), 127.41 (ArCH), 131.19 (ArC), 132.92 (ArC), 135.19 (ArC), 138.12 (ArC), 140.89 (ArC), 145.48 (ArC), 146.93 (ArC), 150.14 (ArC), 150.72 (ArC)

νₘₐₓ (KBr)/cm⁻¹ 3398.5, 2962.3, 1637.2, 1487.8, 1466.1, 1059.1

Melting point: 48-50° C.

### Step 14

### (Z)-8,9-dihydro-5-(3-hydroxy-4-methoxyphenyl)-1,2,3-trimethoxybenzo[7]annulen-7-one 1

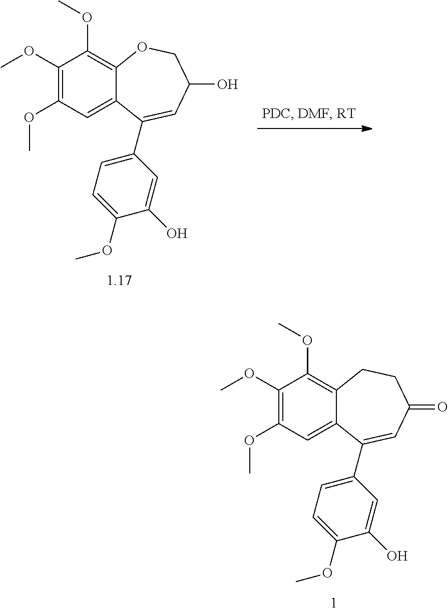 [SMILES: COc1ccc(C2=CC(O)COc3c2cc(OC)c(OC)c3OC)cc1O ; COc1ccc(C2=CC(=O)CCc3c2cc(OC)c(OC)c3OC)cc1O]

To a stirred solution of (Z)-6,7-dihydro-9-(3-hydroxy-4-methoxyphenyl)-2,3,4-trimethoxy-5H-benzo[7]annulen-7-ol 1.17 (140 mg, 0.38 mmol) dissolved in DMF (3 mL) was added pyridinium dichromate (142 mg, 0.75 mmol) at room temperature. The progress of the reaction was monitored by TLC and after 3 h the reaction was quenched by the addition of sat. aq. NaCl (1×15 mL). The product was extracted with diethyl ether (3×20 mL). The organic fractions were combined, dried over MgSO₄, filtered and concentrated in vacuo. The resulting residue was purified by flash column chromatography (stationary phase; silica gel 230-400 mesh, mobile phase; 2:1, hexane/ethyl acetate). All homogenous fractions were collected and the solvent was evaporated to afford (Z)-8,9-dihydro-5-(3-hydroxy-4-methoxyphenyl)-1,2,3-trimethoxybenzo[7]annulen-7-one 1 as a white solid (98 mg, 70%).

¹H NMR (CDCl₃, 400 MHz) δ_{H} ppm: 2.72 (2H, q, J=4.5 Hz, 2.5 Hz, CH₂), 3.14 (2H, t, J=6.0 Hz, CH₂), 3.64 (3H, s, OMe), 3.91 (3H, s, OMe), 3.95 (6H, s, 2×OMe), 5.75 (1H, s, OH), 6.38 (2H, s, ArH {A-ring}& C═CH), 6.89 (3H, m, 3×ArH {C-ring})

¹³C NMR (CDCl₃, 400 MHz) δ_{c} ppm: 19.78 (CH₂), 45.24 (CH₂), 54.54 (OMe), 55.59 (OMe), 60.49 (OMe), 60.98 (OMe), 109.74 (ArCH), 111.43 (ArCH), 114.99 (ArCH), 120.72 (ArCH), 127.68 (ArCH), 128.62 (ArC), 132.02 (ArC), 135.49 (ArC), 142.77 (ArC), 144.78 (ArC), 146.89 (ArC), 149.48 (ArC), 150.62 (ArC), 151.30 (ArC), 203.78 (C═O)

νₘₐₓ (KBr)/cm⁻¹ 3260.6, 2940.5, 1637.5, 1606.6

Melting point: 149-152° C.

Alternative method to synthesise 1 from 1.11

### Synthesis of Intermediate, 9-(3-[1-(tert-butyl)-1,1-dimethylsilyl]oxy-4-methoxyphenyl)-2,3,4-trimethoxy-6,7-dihydro-5H-benzo[a]cyclohepten-7-ol 1.18

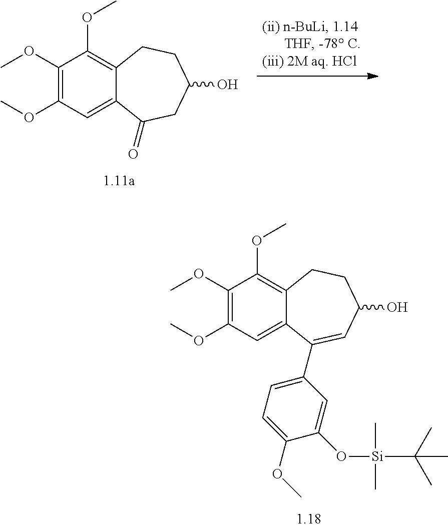 [SMILES: COc1cc2c(c(OC)c1OC)CCC(O)CC2=O ; COc1ccc(C2=CC(O)CCc3c2cc(OC)c(OC)c3OC)cc1O[Si](C)(C)C(C)(C)C]

### Step 1

To a stirred solution of 1.14 (1.40 g, 4.50 mmol) in anhydrous THF (12 mL) was added 2.5M n-BuLi (2.8 mL, 6.80 mmol) dropwise at −78° C., under anhydrous conditions. After 20 min, whilst maintaining the temperature at −78° C., a solution of 1.11a (400 mg, 1.50 mmol) in anhydrous THF (10 mL), was added to the reaction. After 2 h the temperature was allowed to increase to 0° C. and was maintained at this temperature for 20 h. The reaction was quenched by the addition of 2M aq. HCl (1×25 mL). The product was extracted with diethyl ether (3×20 mL). The organic fractions were combined, dried over MgSO₄, filtered and concentrated in vacuo. The resulting residue was purified by flash column chromatography (stationary phase; silica gel 230-400 mesh, mobile phase; 3:1, hexane/ethyl acetate). All homogenous fractions were collected and the solvent was evaporated to afford 1.18 as a yellow oil (440 mg, 60%).

¹H NMR (CDCl₃, 400 MHz) δ_{H} ppm: 0.13 (3H, s, SiCH₃), 0.15 (3H, s, SiCH₃), 0.98 (9H, s, C(CH₃)₃), 2.10-3.03 (4H, m, 2×CH₂), 3.65 (3H, s, OMe {C-ring}), 3.80 (3H, s, OMe), 3.89 (3H, s, OMe), 3.90 (3H, s, OMe), 4.12 (1H, m, CHOH), 6.25 (1H, d, J=5.0 Hz, C═CH), 6.34 (1H, s, Ar{A-ring}), 6.80 (3H, m, ArH{C-ring})

¹³C NMR (CDCl₃, 400 MHz) δ_{c} ppm: −5.00 (Si(CH₃)₂), 17.97 (C(CH₃)₃), 21.34 (CH₂), 25.26 (C(H₃)₃), 42.96 (CH₂), 55.00 (OMe), 55.40 (OMe), 60.40 (OMe), 61.08 (OMe), 69.20 (CHOH), 108.22 (ArCH), 111.16 (ArCH), 120.19 (ArCH), 121.03 (ArCH), 127.52 (ArC), 131.44 (ArCH), 133.61 (ArC), 134.95 (ArC), 137.98 (ArC), 140.85 (ArC), 144.13 (ArC), 150.08 (ArC), 150.21 (ArC), 150.57 (ArC)

νₘₐₓ (DCM)/cm⁻¹ 3411.4, 2931.7, 1596.0

### Synthesis of 9-(3-hydroxy-4-methoxyphenyl)-2,3,4-trimethoxy-6,7-dihydro-5H-benzo[a]cyclohepten-7-one 1

### Step 2

### Oxidation

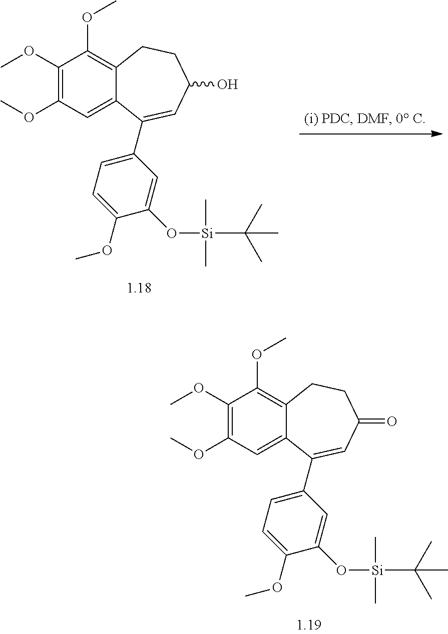

To a stirred solution of 1.18 (200 mg, 0.41 mmol) in DMF (4 mL) was added, pyridinium dichromate (310 mg, 0.82 mmol) at room temperature. The progress of the reaction was monitored by TLC and after 9 h the reaction was quenched by the addition of sat. aq. NaCl (1×15 mL). The product was extracted with diethyl ether (3×20 mL). The organic fractions were combined, dried over MgSO₄, filtered and concentrated in vacuo. The resulting residue was purified by flash column chromatography (stationary phase; silica gel 230-400 mesh, mobile phase; 2:1, hexane/ethyl acetate). All homogenous fractions were collected and the solvent was evaporated to afford 1.19 as a yellow oil (120 mg, 60%).

¹H NMR (CDCl₃, 400 MHz) δ_{H} ppm: 0.16 (6H, s, Si(CH₃)₂), 0.99 (9H, s, C(CH₃)₃), 2.72 (2H, m, CH₂), 3.16 (2H, t, J=5.5 Hz, CH₂), 3.62 (3H, s, OMe), 3.86 (3H, s, OMe), 3.91 (3H, s, OMe), 3.94 (3H, s, OMe), 6.36 (2H, s, ArH {A-ring} & C═CH), 6.84 (2H, m, 2×ArH {C-ring}), 6.92 (1H, dd, J=2.0 Hz, 8.0 Hz, ArH {C-ring})

¹³C NMR (CDCl₃, 400 MHz) δ_{c} ppm: −5.03 (Si(CH₃)₂), 17.99 (C(CH₃)₃), 19.80 (CH₂), 25.22 (C(CH₃)₃), 45.21 (CH₂), 54.96 (OMe), 55.46 (OMe), 60.47 (OMe), 60.96 (OMe), 110.88 (ArCH), 111.34 (ArCH), 121.25 (ArCH), 122.47 (ArCH), 127.47 (ArCH), 128.60 (ArC), 132.16 (ArC), 134.89 (ArC), 142.73 (ArC), 144.13 (ArC), 149.46 (ArC), 150.56 (ArC), 151.31 (ArC), 151.34 (ArC), 203.70 (C═O)

νₘₐₓ (DCM)/cm⁻¹ 2932.8, 1658.8, 1593.1

### Step 3

### Deprotection Step to Furnish 1

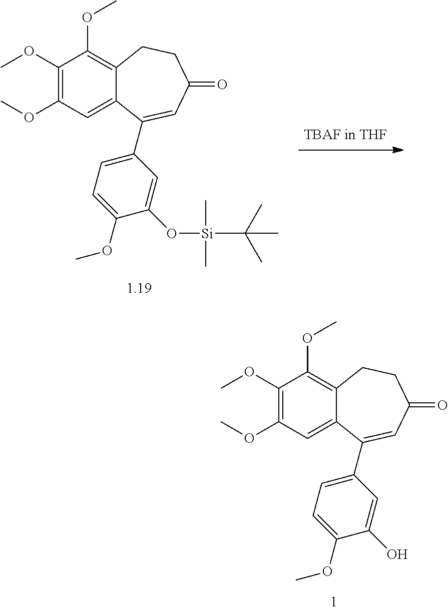

To a stirred solution of 1.19 in THF (0.5 mL) was added 1M TBAF (0.1 mL, 0.10 mmol) drop-wise at 0° C. After 2 h, the reaction was quenched by the addition of water (5 mL) and the product was extracted with diethyl ether (3×5 mL). The organic fractions were collected, dried under sodium sulphate and filtered before the filtrate was concentrated in vacuo. The resulting residue was purified by flash column chromatography (stationary phase; silica gel 230-400 mesh, mobile phase; 1:1 hexane/ethyl acetate). All homogenous fractions were collected and the solvent was evaporated off to afford 1 (Z)-8,9-dihydro-5-(3-hydroxy-4-methoxyphenyl)-1,2,3-trimethoxybenzo[7]annulen-7-one, as a white solid.

### Alternative Synthesis of 9-(3-hydroxy-4-methoxyphenyl)-2,3,4-trimethoxy-5H-benzo[7]annulen-7(6H)-one

### Step 1

### Wittig Reaction Synthesis of (E)-ethyl 3-oxo-5-(2,3,4-trimethoxyphenyl)pent-4-enoate 1.23

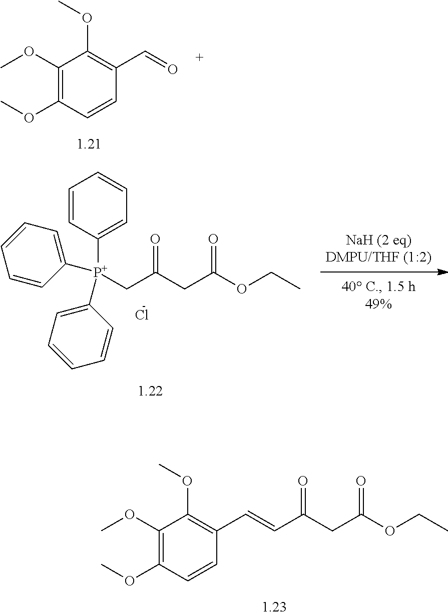

To a stirred solution of [3-(ethoxycarbonyl)-2-oxypropyl]triphenylphosphonium chloride 1.22 (4.35 g, 10.19 mmol) in dry THF (10 mL) and N,N-dimethylpropyleneurea (5 mL) under an atmosphere of nitrogen was added sodium hydride 60% dispersion in mineral oil (0.82 g, 20.39 mmol). After 20 min a solution of 2,3,4-trimethoxybenzaldehyde 1.21 (1.00 g, 5.1 mmol) in dry THF (5 mL) was added to the reaction. The reaction was then heated to 40° C. and left stifling for 90 min. The reaction was quenched by the addition of ammonium chloride saturated aqueous solution (50 mL) and extracted with diethyl ether (3×50 mL). The organic fractions were dried over MgSO₄, filtered and concentrated in vacuo. The resulting oily residue was then subjected to flash column chromatography (stationary phase; silica gel 230-400 mesh, mobile phase; 4:1, hexane/ethyl acetate) to yield a crude bright yellow oil. The mixture containing 1.23 (0.76 g, 49%) was not purified further.

### Step 2

### Reduction: Synthesis of ethyl 3-oxo-5-(2,3,4-trimethoxyphenyl)pentanoate 1.24 and ethyl 3-hydroxy-5-(2,3,4-trimethoxyphenyl)pentanoate (1.25)

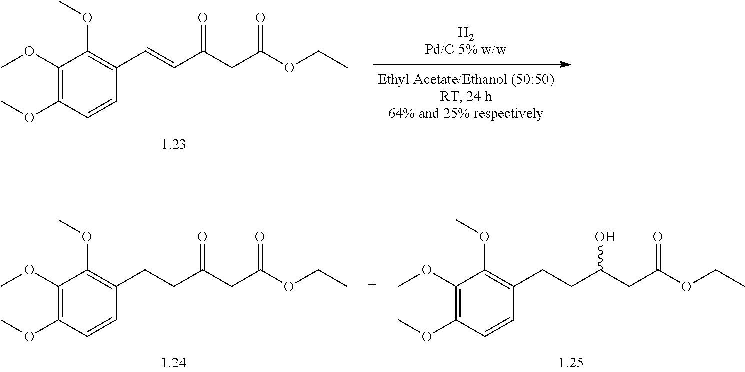 [SMILES: CCOC(=O)CC(=O)/C=C/c1ccc(OC)c(OC)c1OC ; CCOC(=O)CC(=O)CCc1ccc(OC)c(OC)c1OC ; CCOC(=O)CC(O)CCc1ccc(OC)c(OC)c1OC]

To a stirred solution of 1.23 (0.76 g, 2.46 mmol) in a mixture of ethanol (25 mL) and ethyl acetate (25 mL) was added palladium 5% w/w on activated carbon (0.1 g). The reaction was stirred under an atmosphere of hydrogen for 24 h. The palladium on activated carbon was filtered off and the solvent removed in vacuo. The resulting residue was purified by flash column chromatography (stationary phase; silica gel 230-400 mesh, mobile phase; 10:1, hexane/ethyl acetate) to yield (1.24) as a yellow oil (0.49 g, 64%) and (1.25) as a clear oil (0.19 g, 25%).

(1.24)

¹H NMR (CDCl₃, 400 MHz) δ_{H} ppm: 1.17 (3H, t, J=7.27 Hz, CH₃), 2.74 (4H, s, 2×CH₂), 3.36 (2H, s, CH₂), 3.73 (3H, s, OMe), 3.76 (3H, s, OMe), 3.79 (3H, s, OMe), 4.08 (2H, q, J=7.02 Hz, CH₂), 6.50 (1H, d, J=8.7 Hz, ArH), 6.73 (1H, d, J=8.7 Hz, ArH)

¹³C NMR (CDCl₃, 400 MHz) δ_{c} ppm: 13.52 (CH₃), 23.54 (CH₂), 43.22 (CH₂), 48.71 (H₂), 55.34 (CH₃), 60.06 (CH₃), 60.21 (CH₃), 60.66 (CH₂), 106.61 (CH), 123.37 (CH), 125.80 (ArC), 141.64 (ArC), 151.25 (ArC), 151.85 (ArC), 166.64 (C═O), 201.80 (C═O)

vₘₐₓ (DCM)/cm⁻¹: 2979.52, 2938.41, 2836.11, 1743.98, 1716.23, 1602.86

HRMS: calculated 313.3582, found 313.1672, molecular formula (C₁₆H₂₄O₆).

(1.25)

¹H NMR (CDCl₃, 400 MHz) δ_{H} ppm: 1.25 (3H, t, J=7.42 Hz, CH₃), 1.72 (2H, m, CH₂), 2.47 (2H, m, CH₂) 2.69 (2H, m, CH₂), 3.28 (1H, s, CHOH), 3.83 (3H, s, OMe), 3.86 (3H, s, OMe), 3.88 (3H, s, OM), 3.98 (1H, m, CHOH), 4.15 (2H, m, CH₂), 6.60 (1H, d, J=8.5 Hz, ArH), 6.84 (1H, d, J=8.5 Hz, ArH)

¹³C NMR (CDCl₃, 400 MHz) δ_{c} ppm: 13.70 (CH₃), 25.14 (CH₂), 37.20 (CH₂), 40.95 (CH₂), 55.51 (CH₃), 60.15 (CH₂), 60.27 (CH₃), 60.51 (CH₃), 66.78 (CHOH), 106.88 (CH), 123.51 (CH), 127.00 (ArC), 141.70 (ArC), 151.31 (ArC), 151.60 (ArC), 172.39 (C═O)

vₘₐₓ (DCM)/cm⁻¹: 3501.04, 2935.26, 1726.08, 1602.29, 1493.66, 1467.89

HRMS: calculated 335.1471, found 335.1474, molecular formula (C₁₆H₂₄NaO₆).

### Alternative Step 1

### Wittig Reaction Using Commercial Ylide: Synthesis of (E)-ethyl 3-oxo-5-(2,3,4-trimethoxyphenyl)pent-4-enoate 1.23

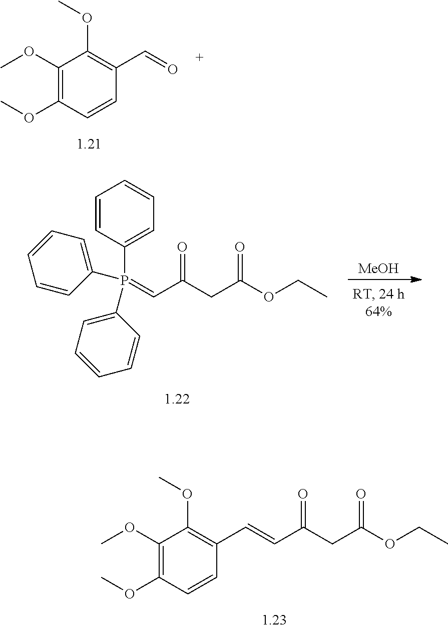

To a stirred solution of 2,3,4-trimethoxybenzaldehyde 1.21 (0.5 g, 2.55 mmol) in methanol (5 mL) was added ethyl 3-oxo-4-(triphenylphosphoranylidene)butyrate 1.22 (1.19 g, 3.06 mmol). After 24 h the solvent was removed under reduced pressure and the resulting residue was subjected to flash column chromatography (stationary phase; silica gel 230-400 mesh, mobile phase; 4:1, hexane/ethyl acetate) to yield 1.23 as constituent of a bright yellow oil (0.5 g, 64%). The product was not purified further.

### Step 3

### Silyl Protection of Alcohol: Synthesis of ethyl 3-(2,2-dimethyl-1,1-diphenylpropoxy)-5-(2,3,4-trimethoxyphenyl)pentanoate 1.27

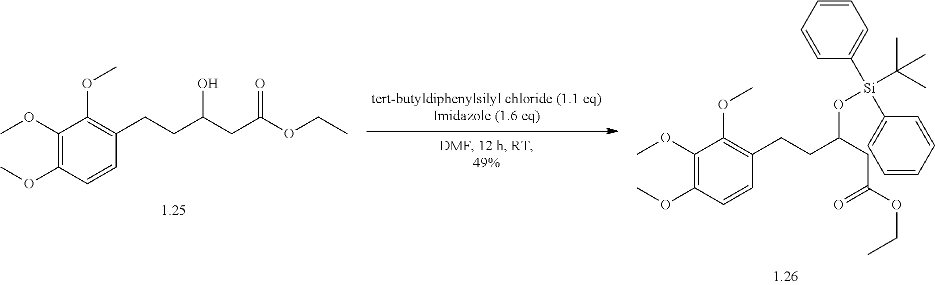 [SMILES: CCOC(=O)CC(O)CCc1ccc(OC)c(OC)c1OC ; CCOC(=O)CC(CCc1ccc(OC)c(OC)c1OC)O[Si](c1ccccc1)(c1ccccc1)C(C)(C)C]

To a stirred solution of 1.25 (0.17 g, 0.57 mmol) in dry DMF (5 mL) under an atmosphere of nitrogen was added imidazole (0.06 g, 0.92 mmol) and tert-butyldiphenylsilyl chloride (0.22 mL, 0.57 mmol). The reaction was left stirring for 12 h and then quenched by the addition of water (50 mL). The reaction mixture was then extracted with diethyl ether (3×25 mL). The organic fractions were dried over MgSO₄, filtered and concentrated in vacuo. The resulting oily residue was then purified by flash column chromatography (stationary phase; silica gel 230-400 mesh, mobile phase; 10:1, hexane/ethyl acetate) to yield (1.26) as bright yellow oil (0.76 g, 49%).

¹H NMR (CDCl₃, 400 MHz) δ_{H} ppm: 1.09 (9H, s, C(CH₃)₃, 1.21 (3H, t, J=7 Hz, CH₃), 1.77 (2H, m, CH₂), 2.49 (2H, m, CH₂), 2.58 (2H, m, CH₂), 3.78 (3H, s, OMe), 3.85 (3H, s, OMe), 3.86 (3H, s, OMe), 4.04 (2H, m, CH₂), 4.31 (1H, m, CHOSi), 6.54 (1H, d, J=9 Hz, ArH), 6.61 (1H, d, J=9 Hz, ArH), 7.41 (6H, m, ArH), 7.73 (4H, m, ArH)

¹³C NMR (CDCl₃, 400 MHz) δ_{c} ppm: 13.66 (CH₃), 18.92 (C(CH₃)₃), 24.61 (CH₂), 26.51 (C(CH₃)₃), 37.77 (CH₂), 41.58 (CH₂), 55.53 (OMe), 59.84 (OMe), 60.25 (OMe), 60.34 (OMe), 69.91 (CHOSi), 106.63 (ArCH), 123.06 (ArCH), 127.06 (2×ArCH), 127.09 (2×ArCH), 127.47 (2×QC), 129.16 (2×ArCH), 133.58 (QC), 135.47 (2×ArCH), 135.52 (2×ArCH), 141.70 (QC), 151.30 (QC), 151.42 (QC), 171.06 (C═O)

vₘₐₓ (DCM)/cm⁻¹: 2960.35, 2934.16, 2091.36, 1734.62, 1644.78, 1494.76, 1466.34

HRMS: [M+K⁺] calculated 573.2618, found 573.2623, molecular formula (C₃₃H₄₂O₆K).

### Step 4

### Hydrolysis of Ethyl Ester: Synthesis of 3-(2,2-dimethyl-1,1-diphenylpropoxy)-5-(2,3,4-trimethoxyphenyl)pentanoic acid 1.8

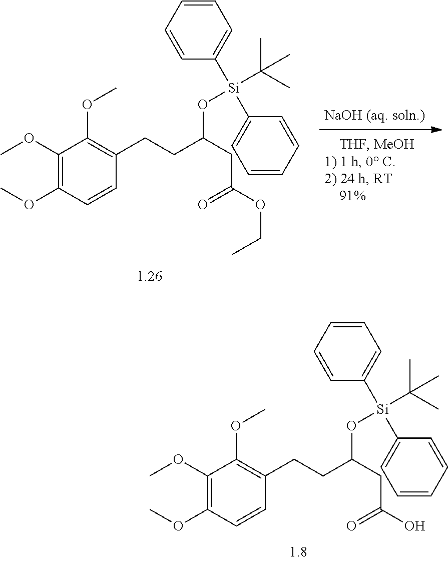 [SMILES: CCOC(=O)CC(CCc1ccc(OC)c(OC)c1OC)O[Si](c1ccccc1)(c1ccccc1)C(C)(C)C ; COc1ccc(CCC(CC(=O)O)O[Si](c2ccccc2)(c2ccccc2)C(C)(C)C)c(OC)c1OC]

To a stirred solution of 1.26 (0.09 g, 0.17 mmol) in a mixture of methanol (0.85 mL) and THF (0.66 mL) at 0° C. was added 2.5M NaOH solution (0.25 mL) dropwise. After 24 h the pH of the solution was adjusted to pH 7 by the addition of 2M HCl aqueous solution. The organic solvent was removed under reduced pressure and the aqueous phase was extracted with diethyl ether (3×10 mL). The organic fractions were dried over MgSO₄, filtered and concentrated in vacuo. The resulting residue was then purified by flash column chromatography (stationary phase; silica gel 230-400 mesh, mobile phase; 2:1, hexane/ethyl acetate) to yield 1.8 as a white solid (0.08 g, 91%).

Data Described Previously

### 4.3 Synthesis of Enantiomerically Pure (S)-9-(3-hydroxy-4-methoxyphenyl)-2,3,4-trimethoxy-6,7-dihydro-5H-benzo[7]annulen-7-ol S-(1.02)

### Bioreduction: Synthesis of (S)-methyl 3-hydroxy-5-(2,3,4-trimethoxyphenyl)pentanoate S-1.10

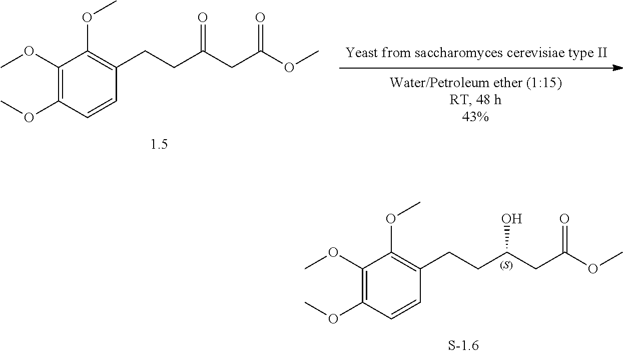

To a clean 250 mL round bottomed flask was added 1.5 (1 g, 3.37 mmol), yeast from saccharomyces cerevisiae type II (10 g), water (10 mL) and petroleum ether (150 mL). The flask was stoppered and shaken for 48 h. The supernatant was decanted through filter paper and the yeast extracted with ethyl acetate (3×100 mL). The organic fractions were combined, dried over MgSO₄ and concentrated in vacuo. The resulting oily residue was then purified by flash column chromatography (stationary phase; silica gel 230-400 mesh, mobile phase; 4:1, hexane/ethyl acetate) to yield S-1.6 as clear colourless oil (0.43 g, 43%).

[α]_{D}²⁵−10.70

Remainder of synthesis accomplished as for racemic 1.6 to yield S-1.01

### Bioreduction: Synthesis of (S)-ethyl 3-hydroxy-5-(2,3,4-trimethoxyphenyl)pentanoate S-1.21

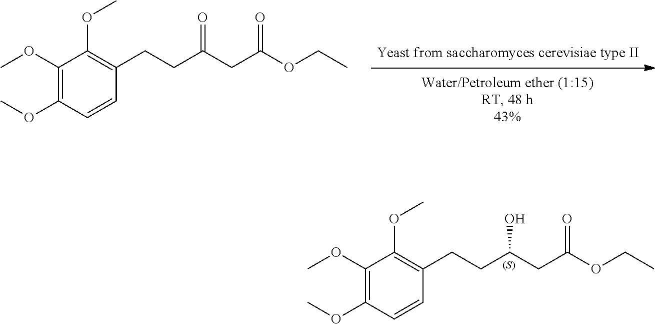

To a clean 250 mL round bottomed flask was added 1.24 (0.45 g, 1.45 mmol), yeast from Saccharomyces cerevisiae, type II (4 g), water (6 mL) and petroleum ether (100 mL). The flask was stoppered and shaken for 48 h. The supernatant was decanted through filter paper and the yeast extracted with ethyl acetate (3×100 mL). The organic fractions were combined, dried over MgSO₄ and concentrated in vacuo. The resulting oily residue was then purified by flash column chromatography (stationary phase; silica gel 230-400 mesh, mobile phase; 4:1, hexane/ethyl acetate) to yield S-1.25 as a clear colourless oil (0.23 g, 51%). Data as described for racemic 1.6 above.

### Formation of 9-(3,5-dihydroxy-4-methoxyphenyl)-2,3,4-trimethoxy-6,7-dihydro-5H-benzo[a]cyclohepten-7-one 2

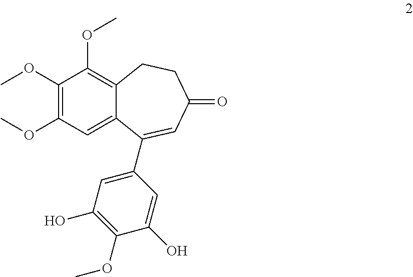 [SMILES: COc1cc2c(c(OC)c1OC)CCC(=O)C=C2c1cc(O)c(OC)c(O)c1]

### 1^{st} Step

### Synthesis of Intermediate, 5-bromo-2-methoxy-1,3-benzenediol 2.2

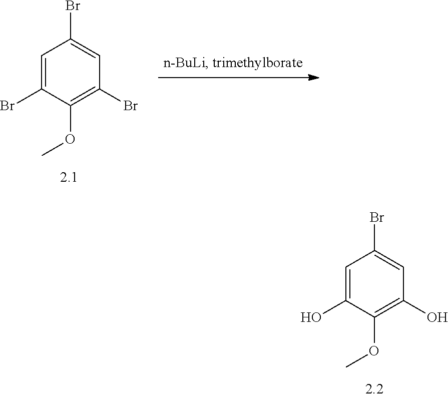

To a stirred suspension of 1,3,5-tribromo-2-methoxybenzene 2.1 (5.0 g, 14.5 mmol) in anhydrous pentane (100 mL) was added 2.5M n-BuLi (28.96 mL, 72.4 mmol) at −20° C. under anhydrous conditions, over a 10-min period. The solution was allowed to warm to −15° C. over 15 min. Under subsequent cooling to −30° C., trimethylborate (8.55 mL, 72.4 mmol) was added all at once. The reaction was subsequently warmed to 0° C. over 30 min and then cooled to −10° C. To this was added 40% solution of peracetic acid/acetic acid (15 mL) over a period of 30 min. Upon completion of the addition, the solution was warmed to 0° C. over 30 min and re-cooled to −10° C. whereupon saturated aqueous NaHSO₃ (15 mL) was added over 30 min. On completion, water (100 mL) was added and the product was extracted with diethyl ether (3×100 mL). The ether fractions were collected, dried over sodium sulphate, filtered and concentrated to an oily residue under reduced pressure. It was purified by flash column chromatography (stationary phase: silica gel G₂₅₄; mobile phase: hexane/ethyl acetate 3:1). All homogeneous fractions were collected and the solvent was removed in vacuo to afford 2.2 as a red solid (2.00 g, 63%). ¹H NMR (CDCl₃, 400 MHz) δ_{H} ppm 3.84 (3H, s, OMe), 5.84 (2H, br, s, 2×OH), 6.56 (1H, d, J=2 Hz, ArH), 6.77 (1H, d, J=2 Hz, ArH). ¹³C NMR δ_{c} ppm 24.47 (CH₂), 59.49 (OMe), 114.62 (qC), 117.58 (2×ArCH), 142.04 (qC), 150.14 (2×qC).

### 2^{nd} Step

### Synthesis of Intermediate, (5-bromo-3-[1-(tert-butyl)-1,1-dimethylsilyl]oxy-2-methoxyphenoxy)(tert-butyl)dimethylsilane 2.3

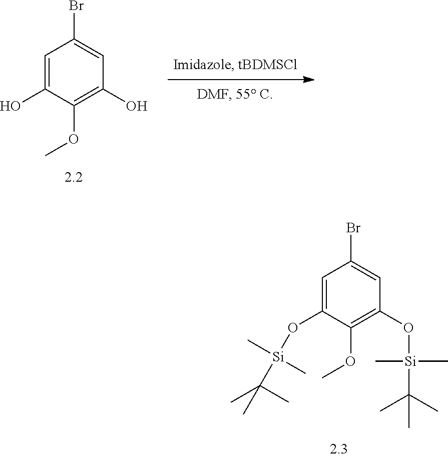 [SMILES: COc1c(O)cc(Br)cc1O ; COc1c(O[Si](C)(C)C(C)(C)C)cc(Br)cc1O[Si](C)(C)C(C)(C)C]

To a stirred solution of 2.2 (3.68 g, 16.8 mmol) in DMF (10 mL) was added imidazole (6.28 g, 92.2 mmol) and tBDMSCl (5.57 g, 36.9 mmol) at 25° C. After 1 hour, the reaction temperature was raised to 55° C. and was allowed to proceed at this temperature for 10 h. On completion, the reaction was quenched by the addition of sat. aq. NaCl (25 mL) and the product extracted with diethyl ether (3×25 mL). The organic extracts were collected, dried over sodium sulphate, filtered, and then concentrated to an oil. It was purified by flash column chromatography (stationary phase: silica gel G₂₅₄; mobile phase: hexane/ethyl acetate 9:1). All homogeneous fractions were collected and the solvent was removed in vacuo to afford 2.3 as a white waxy solid (5.65 g, 75%). M.pt. 52-54° C. νₘₐₓ (KBr)/cm⁻¹ 2930.5, 1574.3, 1482.6, 1085.2, 1011.0. GCMS m/z (%) 447 (100), 375 (94), 73 (99). ¹H NMR (CDCl₃, 400 MHz) δ_{H} ppm 0.12 (6H, s, CH₃SiCH₃), 0.23 (6H, s, CH₃SiCH₃), 0.93 (9H, s, C(CH₃)₃), 1.02 (9H, s, C(CH₃H₃)₃), 3.84 (3H, s, OMe), 6.56 (1H, d, J 2 Hz, ArH), 6.77 (1H, d, J 2 Hz, ArH). ¹³C NMR δ_{c} ppm −5.12 (2×CH₃SiCH₃), 17.86 (2×C(CH₃)₃), 25.20 (2×C(CH₃)₃), 59.49 (OMe), 114.62 (qC), 117.58 (2×ArCH), 142.04 (qC), 150.14 (2×qC).

### 3^{rd} Step

### Synthesis of 5-(7-hydroxy-2,3,4-trimethoxy-6,7-dihydro-5H-benzo[a]cyclohepten-9-yl)-2-methoxy-1,3-benzenediol 2.4 via 9-(3,5-di[1-(tert-butyl)-1,1-dimethylsilyl]oxy-4-methoxyphenyl)-2,3,4-trimethoxy-6,7-dihydro-5H-benzo[a]cyclohepten-7-ol 2.5

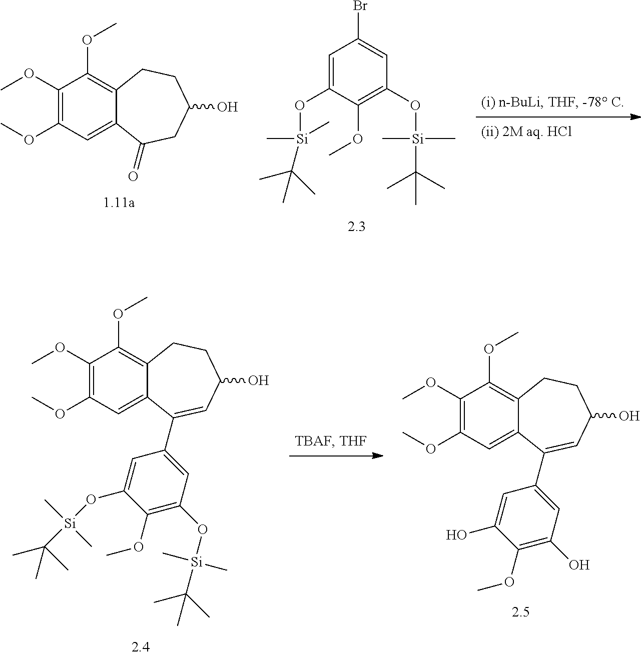 [SMILES: COc1cc2c(c(OC)c1OC)CCC(O)CC2=O ; COc1c(O[Si](C)(C)C(C)(C)C)cc(Br)cc1O[Si](C)(C)C(C)(C)C ; COc1cc2c(c(OC)c1OC)CCC(O)C=C2c1cc(O[Si](C)(C)C(C)(C)C)c(OC)c(O[Si](C)(C)C(C)(C)C)c1 ; COc1cc2c(c(OC)c1OC)CCC(O)C=C2c1cc(O)c(OC)c(O)c1]

To a stirred solution of bromide 2.3 (0.60 g, 1.34 mmol) in anhydrous THF (1 mL) was added 2.5 M n-BuLi (0.54 mL, 1.34 mmol) drop-wise at −78° C. under anhydrous conditions during over 10 min. This was followed by the addition of 1.11a (0.12 g, 0.45 mmol) dissolved in anhydrous THF (1 mL) was added. After 4 h, the temperature was raised to 0° C. and maintained at this temperature for 12 h. On completion, the reaction was quenched by the addition of 2M aq. HCl (5 mL) and the product was extracted using diethyl ether (3×10 mL). The combined ether extracts were dried over sodium sulphate, filtered and the filtrate concentrated to afford 2.4 as a clear oil. 2.4 (0.04 g, 0.065 mmol) was re-dissolved in THF (0.5 mL) was added 1M TBAF (0.065 mL, 0.065 mmol) at room temperature. After 1 hour, the reaction was quenched by the addition of water (2 mL) and the product was extracted with diethyl ether (3×5 mL). The ether extracts were collected, dried over sodium sulphate and reduced in volume before being purified by flash column chromatography (stationary phase: silica gel 230-400 mesh, mobile phase: hexane/ethyl acetate 1:1). All homogenous fractions were collected and the solvent was removed in vacuo to afford 2.5 as a white solid (0.012 g, 48%). M.pt. 60-62° C. νₘₐₓ (CCl₄)/cm⁻¹ 3400.2, 2926.2, 1638.4, 1591.0. HRMS: found 411.1433 (M⁺+Na), requires (C₂₁H₂₄O₇) 388.1522. GCMS m/z (%) 370 (M⁺−18, 100), 323 (20). ¹H NMR (CD₃OD, 400 MHz) δ_{H} ppm 2.11 (1H, m, HCH), 2.33 (1H, m, HCH), 2.50 (1H, m, HCH), 3.02 (1H, m, HCH), 3.71 (3H, s, OMe), 3.91 (3H, s, OMe), 3.93 (3H, s, OMe), 3.93 (3H, s, OMe), 4.16 (1H, m, CHOH), 6.29 (1H, d, J 5.0 Hz, C═CH), 6.38 (1H, s, {A-ring}ArH), 6.47 (2H, s, 2×{C-ring}ArH). ¹³C NMR δ_{c} ppm 21.26 (CH₂), 42.79 (CH₂), 55.67 (OMe), 60.40 (OMe), 60.63 (OMe), 61.08 (OMe), 69.37 (CHOH), 107.36 (2×{C-ring}ArCH), 108.55 ({A-ring}ArCH), 127.47 (qC), 132.01 (C═CH), 133.80 (qC), 134.25 (qC), 137.30 (qC), 138.12 (qC), 148.26 (2×qC), 150.28 (qC), 150.77 (qC).

### 4^{th} Step

### Synthesis of Intermediate, 9-(3,5-di[1-(tert-butyl)-1,1-dimethylsilyl]oxy-4-methoxyphenyl)-2,3,4-trimethoxy-6,7-dihydro-5H-benzo[a]cyclohepten-7-one 2.6

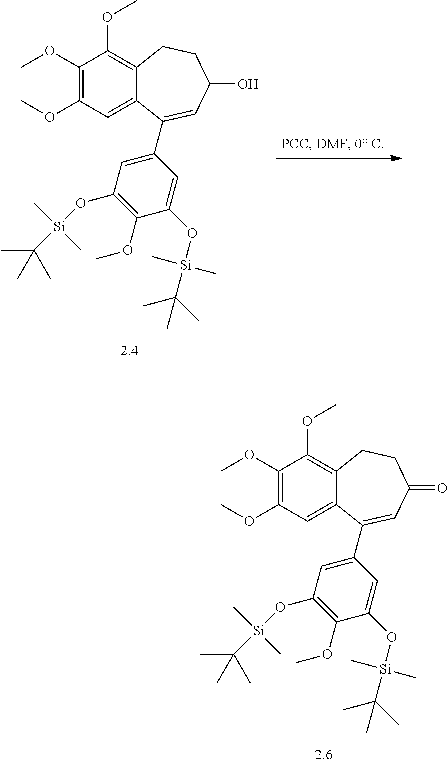 [SMILES: COc1cc2c(c(OC)c1OC)CCC(O)C=C2c1cc(O[Si](C)(C)C(C)(C)C)c(OC)c(O[Si](C)(C)C(C)(C)C)c1 ; COc1cc2c(c(OC)c1OC)CCC(=O)C=C2c1cc(O[Si](C)(C)C(C)(C)C)c(OC)c(O[Si](C)(C)C(C)(C)C)c1]

To a stirred solution of 2.4 (0.10 g, 0.16 mmol) in DMF (1 mL) was added PDC (0.061 g, 0.16 mmol) at 0° C. After 24 h, the reaction was quenched by the addition of water (5 mL) and the product was extracted with diethyl ether (5×5 mL). The organic fractions were collected, dried over sodium sulphate and filtered before being concentrated in vacuo. The resulting residue was purified by flash column chromatography (stationary phase; silica gel 230-400 mesh, mobile phase; 1:1 hexane/ethyl acetate). All homogenous fractions were collected and the solvent was evaporated to afford 2.6 as a clear oil (0.05 g, 50%). ¹H NMR (CDCl₃, 400 MHz) δ_{H} ppm 0.17 (12H, s, 2×CH₃SiCH₃), 1.00 (18H, s, C(CH₃)₃), 2.72 (2H, m, CH₂), 3.14 (2H, m, CH₂), 3.64 (3H, s, OMe), 3.79 (3H, s, OMe), 3.92 (3H, s, OMe), 3.94 (3H, s, OMe), 6.31 (1H, s, C═CH), 6.35 (1H, s, ArH), 6.46 (2H, m, 2×ArH). ¹³C NMR δ_{c} ppm −5.05 (2×CH₃SiCH₃), 17.85 (C(CH₃)₃), 19.80 (CH₂), 25.22 (C(CH₃)₃), 45.17 (CH₂), 55.42 (OMe), 55.57 (OMe), 60.43 (OMe), 60.94 (OMe), 111.34 (ArCH), 115.06 (2×ArCH), 125.67 (C═CH), 128.50 (qC), 132.04 (qC), 137.48 (ArCH), 143.18 (qC), 149.08 (2×qC), 149.30 (qC), 150.63 (qC), 203.68 (C═O).

### 5th Step-Deprotection

### Synthesis of 9-(3,5-dihydroxy-4-methoxyphenyl)-2,3,4-trimethoxy-6,7-dihydro-5H-benzo[a]cyclohepten-7-one 2

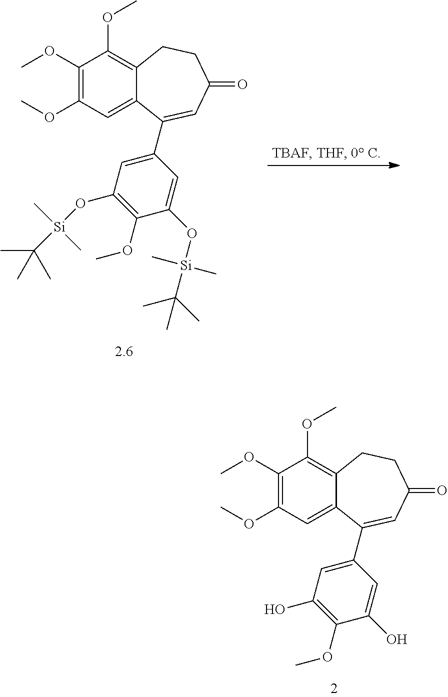

To a stirred solution of 2.6 (0.05 g, 0.08 mmol) in THF (0.5 mL) was added 1M TBAF (0.1 mL, 0.10 mmol) drop-wise at 25° C. After 2 h, the reaction was quenched by the addition of water (5 mL) and the product was extracted with diethyl ether (3×5 mL). The organic fractions were collected, dried over sodium sulphate and filtered before the filtrate was concentrated in vacuo. The resulting residue was purified by flash column chromatography (stationary phase; silica gel 230-400 mesh, mobile phase; 1:1 hexane/ethyl acetate). All homogenous fractions were collected and the solvent was evaporated to afford 2 as an oil (0.03 g, 95%). HRMS: found 387.1481 (MH⁺), requires (C₂₁H₂₂O₇) 386.1366. ¹H NMR (CDCl₃, 400 MHz) δ_{H} ppm 2.73 (2H, m, CH₂), 3.13 (2H, m, CH₂), 3.66 (3H, s, OMe), 3.90 (3H, s, OMe), 3.95 (3H, s, OMe), 3.98 (3H, s, OMe), 5.83 (2H, br, 2×OH), 6.37 (1H, s, C═CH), 6.40 (1H, s, ArH), 6.53 (2H, s, 2×ArH). ¹³C NMR δ_{c} ppm 19.74 (CH₂), 43.16 (CH₂), 55.71 (OMe), 60.45 (OMe), 60.69 (OMe), 60.94 (OMe), 108.67 (2×ArCH), 111.60 (ArCH), 127.94 (C═CH), 128.54 (qC), 131.62 (qC) 134.77 (qC), 138.67 (qC), 142.99 (qC), 148.23 (2×qC), 149.51 (qC), 150.74 (qC), 151.29 (qC), 203.98 (C═O).

### Formation of 2,3,4-trimethoxy-9-(4-methoxyphenyl)-6,7-dihydro-5H-benzo[a]cyclohepten-7-one 3

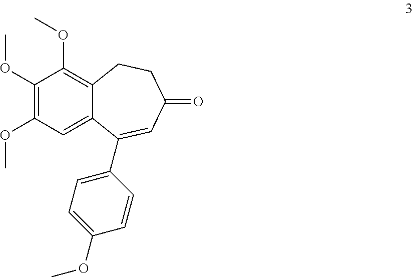 [SMILES: COc1ccc(C2=CC(=O)CCc3c2cc(OC)c(OC)c3OC)cc1]

### Step 1

### Synthesis of 2,3,4-trimethoxy-9-(4-methoxyphenyl)-6,7-dihydro-5H-benzo[a]cyclohepten-7-ol 3

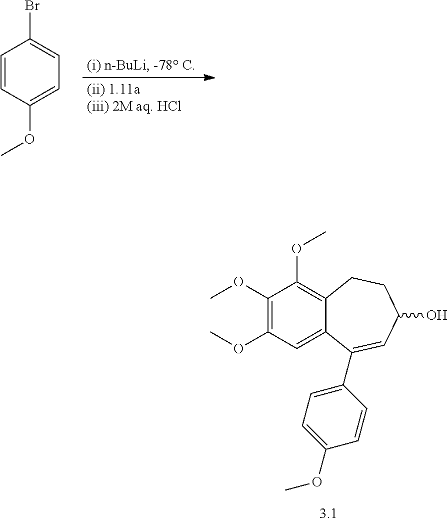

To a stirred solution of para-bromoanisole (0.21 g, 1.12 mmol) in anhydrous THF (2 mL) was added 2.5M n-BuLi (0.45 mL, 1.12 mmol) at −78° C. under anhydrous conditions. After 20 min, whilst maintaining the temperature at −78° C., keto-alcohol 1.11a (0.10 g, 0.37 mmol) dissolved in anhydrous THF (2 mL), was added. The reaction was allowed to continue for 30 min before being quenched by the addition of 2M aq. HCl (6 mL) and the product was extracted with diethyl ether (3×6 mL). The organic fractions were collected, dried over sodium sulphate and filtered before the filtrate was concentrated in vacuo. The resulting residue was purified by flash column chromatography (stationary phase; silica gel 230-400 mesh, mobile phase; 1:1 hexane/ethyl acetate). All homogenous fractions were collected and reduced in volume to afford 3.1 as a white solid (0.084 g, 63%). M.pt. 44-45° C. νₘₐₓ (CCl₄)/cm⁻¹ 3398.2, 2933.4, 1606.4, 1510.0, 1113.9. GCMS m/z (%) 356 (2), 338 (100). HRMS: found 379.1488 (M⁺+Na), requires (C₂₁H₂₄O₅) 356.1624. ¹H NMR (CD₃OD, 400 MHz) δ_{H} ppm 2.06 (1H, m, CH), 2.30 (1H, m, CH), 2.44 (1H, m, CH), 3.05 (1H, m, CH), 3.65 (3H, s, OMe), 3.82 (3H, s, OMe), 3.88 (3H, s, OMe), 3.90 (3H, s, OMe), 4.02 (1H, s, CHOH), 6.23 (1H, m, C═CH), 6.37 (1H, s, ArH), 6.89 (2H, dd, J 2 Hz, 6.5 Hz, 2×ArH), 7.22 (2H, dd, J 2 Hz, 6.5 Hz, 2×ArH). ¹³C NMR δ_{c} ppm 21.00 (CH₂), 42.33 (CH₂), 53.87 (OMe), 54.59 (OMe), 59.39 (OMe), 60.19 (OMe), 68.43 (CHOH), 108.25 (ArCH), 112.90 (2×ArCH), 127.51 (qC), 128.27 (2×ArCH), 130.84 (C═CH), 133.20 (qC), 135.23 (qC), 138.04 (qC), 140.94 (qC), 150.21 (qC), 150.81 (qC), 158.94 (qC).

### Synthesis of 2,3,4-trimethoxy-9-(4-methoxyphenyl)-6,7-dihydro-5H-benzo[a]cyclohepten-7-one 3

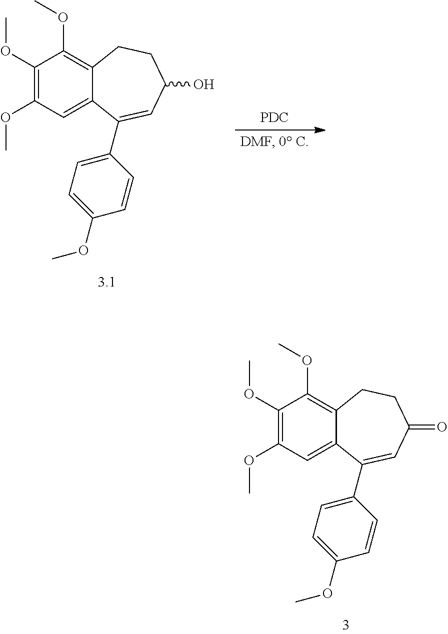

To a stirred solution of 3.1 (0.05 g, 0.14 mmol) in DMF (1 mL) was added PDC (0.10 g, 0.27 mmol) portion-wise at 0° C. After 12 h, the reaction was quenched by the addition of water (5 mL) and the product was extracted using diethyl ether (5×5 mL). The organic fractions were collected, dried over sodium sulphate and filtered before being concentrated in vacuo. The resulting residue was purified by flash column chromatography (stationary phase; silica gel 230-400 mesh, mobile phase; 1:1 hexane/ethylacetate). All homogenous fractions were collected and the solvent was evaporated to afford 3 as a white solid (0.03 g, 60%). M.pt 26-27° C. νₘₐₓ (CCl₄)/cm⁻¹ 2935.4, 1657.7, 1603.8, 1509.8, 1116.4. HRMS: found 355.1571 (MH⁺), requires (C₂₁H₂₂O₅) 354.1467. GCMS m/z (%) 354 (100), 312 (15), 121 (22). ¹H NMR (CD₃OD, 400 MHz) δ_{H} ppm 2.67 (2H, m, CH₂), 3.15 (2H, m, CH₂), 3.59 (3H, s, OMe), 3.85 (3H, s, OMe), 3.89 (6H, s, 2×OMe), 6.33 (1H, d, J 2.5 Hz, C═CH), 6.40 (1H, d, J 2.5 Hz, ArH), 6.97 (2H, dd, J 9.0 Hz, 2.5 Hz, 2×ArH), 7.26 (2H, dd, J 9.0 Hz, 2.5 Hz, 2×ArH). ¹³C NMR δ_{c} ppm 19.37 (CH₂), 44.65 (CH₂), 53.99 (OMe), 54.59 (OMe), 59.44 (OMe), 60.08 (OMe), 111.53 (ArCH), 113.04 (2×ArCH), 126.93 (C═CH), 128.68 (qC), 129.63 (2×ArCH), 131.97 (qC), 134.25 (qC), 143.01 (qC), 149.48 (qC), 150.77 (qC), 152.27 (qC), 160.29 (qC), 204.42 (C═O).

Bromination of (2.18) with PTAB

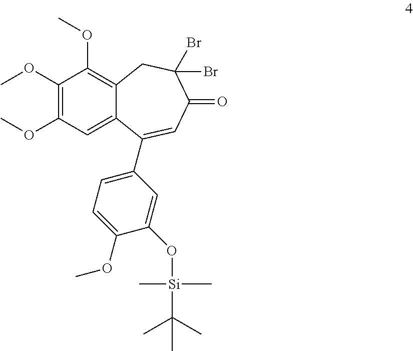 [SMILES: COc1ccc(C2=CC(=O)C(Br)(Br)Cc3c2cc(OC)c(OC)c3OC)cc1O[Si](C)(C)C(C)(C)C]

### Bromination of 1.19

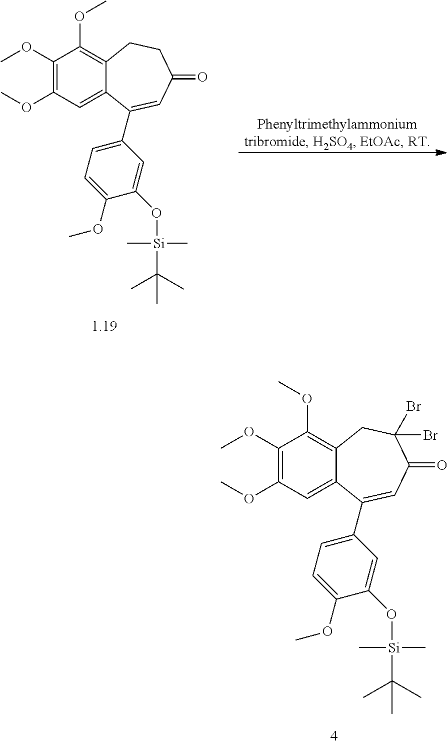

### Synthesis A

To a stirred solution of 1.19 (120 mg, 0.25 mmol) in ethyl acetate (5 mL) was added conc. H₂SO₄ (0.008 mL) in ethyl acetate (0.08 mL). Phenyltrimethylammonium tribromide (0.13 g, 0.33 mmol) was added to the stirred solution. After 90 min the reaction was quenched by the addition of 5% aq. NaHCO₃ (1×30 mL) and the product was extracted with ethyl acetate (2×50 mL). The combined organic extracts were washed with sat. aq. NaCl (1×50 mL). The organic fraction was dried over MgSO₄, filtered and concentrated under vacuum. The resulting residue was purified by flash column chromatography (stationary phase; silica gel 230-400 mesh, mobile phase; 4:1, hexane/ethyl acetate). All homogenous fractions were collected and the solvent was evaporated to afford 4 as a yellow oil (180 mg, c.100%).

### Synthesis B

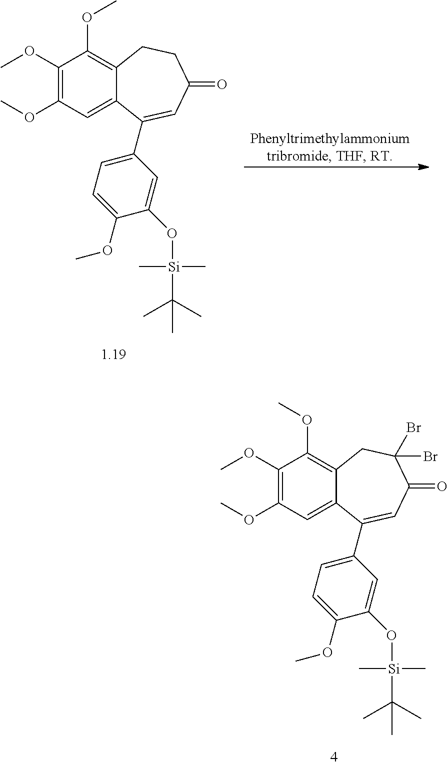

To a stirred solution of 1.19 (50 mg, 0.10 mmol) in anhydrous THF (12 mL) was added phenyltrimethylammonium tribromide (0.04 g, 0.11 mmol) at room temperature, in the dark. After 7 h the reaction was quenched by the addition of 5% aq. NaHCO₃ (1×50 mL) and the product was extracted with diethyl ether (2×50 mL). The combined organic extracts were washed with sat. aq. NaCl (1×50 mL). The organic fraction was dried over MgSO₄, filtered and concentrated under vacuum to afford 4 as a yellow oil (70 mg, c.100%). The resulting residue was not purified.

¹H NMR (CDCl₃, 400 MHz) δ_{H} ppm: 0.16 (6H, s, Si(CH₃)₂), 0.99 (9H, s, C(CHH₃)₃), 3.65 (3H, s, OMe), 3.87 (3H, s, OMe), 3.93 (3H, s, OMe), 3.97 (1H, q, 18.5 Hz, CH₂), 4.08 (3H, s, OMe), 6.33 (1H, s, C═CH), 6.51 (1H, s, ArH {A-ring}), 6.83 (2H, m, 2×ArH {C-ring}), 6.93 (1H, dd, J=2.5 Hz, 8.5 Hz, ArH {C-ring})

¹³C NMR (CDCl₃, 400 MHz) δ_{c} ppm: −5.01 (Si(HH₃)₂), 17.99 (H(CH₃)₃), 25.21 (C(HH₃)₃), 42.57 (HH₂), 54.99 (OMe), 55.42 (OMe), 60.41 (OMe), 61.02 (OMe), 70.26 (QC), 109.80 (ArCH), 110.98 (ArCH), 121.13 (2×ArCH), 122.80 (C═HH), 133.47 (ArC), 132.98 (ArC), 142.49 (ArC), 144.32 (ArC), 150.98 (ArC), 151.06 (ArC), 151.90 (ArC), 152.17 (ArC), 190.74 (C═O)

νₘₐₓ (DCM)/cm⁻¹ 3413.5, 2917.2, 1732.6, 1265.7. 738.1, 703.7

### Synthesis of (Z)-8-bromo-8,9-dihydro-5-(3-hydroxy-4-methoxyphenyl)-1,2,3-trimethoxybenzo[7]annulen-7-one 5 and (5Z,8Z)-5-(3-hydroxy-4-methoxyphenyl)-1,2,3-trimethoxy-7H-benzo[7]annulen-7-one 6

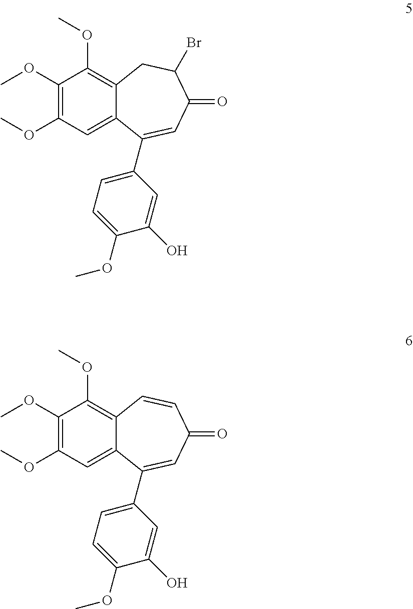 [SMILES: COc1ccc(C2=CC(=O)C(Br)Cc3c2cc(OC)c(OC)c3OC)cc1O ; COc1ccc(-c2cc(=O)ccc3c(OC)c(OC)c(OC)cc23)cc1O]

### Step 1

### Bromination of 1.19 with 5,5-dibromo-2,2-dimethyl-4,6-dioxo-1,3-dioxane

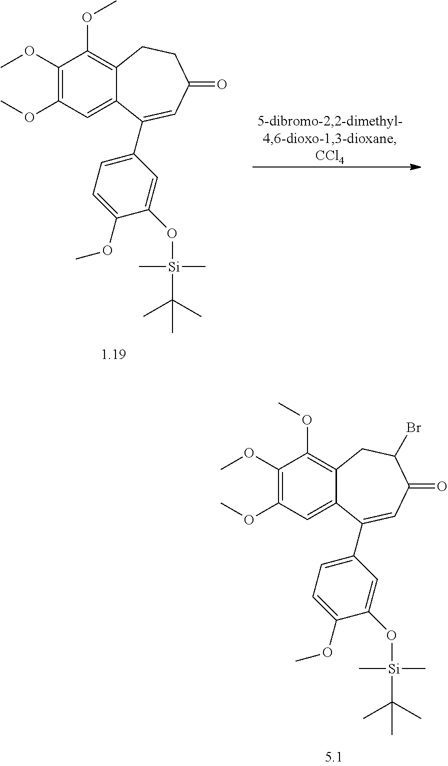

To a stirred solution of 1.19 (100 mg, 0.20 mmol) in CCl₄ (2 mL) was added 5,5-dibromo-2,2-dimethyl-4,6-dioxo-1,3-dioxane (0.06 g, 0.20 mmol). The resulting mixture was refluxed for 30 min. The reaction was quenched by the addition of 5% aq. NaHCO₃ (40 mL) and the product was extracted with diethyl ether (2×40 mL). The combined organic extracts were washed with sat. aq. NaCl (1×50 mL). The organic fraction was dried over MgSO₄, filtered and concentrated under vacuum to afford 5.1 as a yellow oil (100 mg, 85%). The resulting residue was not purified by flash column chromatography.

¹H NMR (CDCl₃, 400 MHz) δ_{H} ppm: 0.17 (6H, s, Si(CH₃)₂), 1.00 (9H, s, C(CH₃)₃), 3.64 (3H, s, OMe), 3.88 (3H, s, OMe), 3.90 (2H, d, J=11.0 Hz, CH₂), 3.95 (3H, s, OMe), 3.99 (3H, s, OMe), 4.64 (1H, d, J=9.0 Hz, CHBr), 6.37 (1H, s, C═CH), 6.42 (1H, s, ArH {A-ring}), 6.87 (3H, m, 3×ArH {C-ring})

¹³C NMR (CDCl₃, 400 MHz) δ_{c} ppm: −5.02 (Si(CH₃)₂) 20.00 (C(CH₃)₃), 25.25 (C(CH₃)₃), 30.37 (CH₂), 52.98 (CHBr), 54.98 (OMe), 55.41 (OMe), 60.41 (OMe), 60.95 (OMe), 106.15 (ArCH), 110.86 (ArCH), 121.22 (ArCH), 122.56 (ArCH), 123.96 (ArC), 124.57 (C═CH), 126.54 (ArC), 132.26 (ArC), 134.01 (ArC), 142.76 (ArC), 144.21 (ArC), 150.69 (ArC), 151.41 (ArC), 151.63 (ArC), 191.11 (C═O)

νₘₐₓ (DCM)/cm⁻¹ 3413.9, 2932.2, 1733.5, 1266.7. 739.0

### Step 2

### Silyl Deprotection and Elimination to Form 5 and 6 Respectively

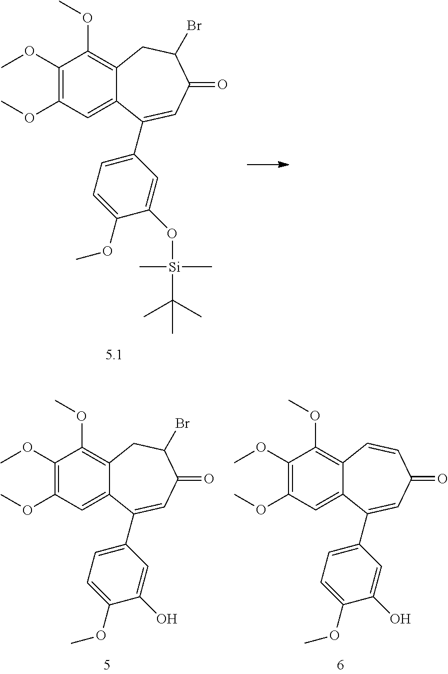

To a stirred solution of the bromide 5.1 (70 mg, 0.13 mmol) was added NaN₃ (8 mg, 10 mmol) in DMF (2 mL) at room temperature. The reaction was left stifling overnight and was quenched by the addition of water (1×20 mL). The product was extracted with diethyl ether (3×20 mL). The combined organic extracts were dried over MgSO₄, filtered and concentrated to an oil in vacuo. The presence of an azide in the product was assessed by IR spectroscopy of the crude mixture. The products were purified by either flash column chromatography, preparatory TLC or a combination of the two methods.

6 ¹H NMR (CDCl₃, 400 MHz) δ_{H} ppm: 3.67 (3H, s, OMe), 3.98 (3H, s, OMe), 3.99 (3H, s, OMe), 4.00 (3H, s, OMe), 6.83 (1H, s, ArH {A-ring}), 6.86 (1H, dd, J=2.0 Hz, 13.0 Hz, CH═CHCO), 6.89 (1H, d, J=2.0 Hz, ArH {C-ring}), 6.92 (1H, d, J=3.0 Hz, C═CH), 6.94 (2H, m, 2×ArH {C-ring}), 8.14 (1H, d, J=13.0 Hz, CH═CHCO))

¹³C NMR (CDCl₃, 400 MHz) δ_{c} ppm: 55.70 (OMe), 55.99 (OMe), 60.06 (OMe), 61.90 (OMe), 110.35 (ArCH), 112.04 (ArCH), 115.37 (ArCH), 120.79 (ArCH), 125.40 (ArC), 132.46 (ArCH), 133.56 (ArC), 134.29 (ArCH), 136.63 (ArCH), 136.98 (ArC), 143.44 (ArC), 145.29 (ArC), 146.41 (ArC), 151.09 (ArC), 152.83 (ArC), 153.81 (ArC), 188.09 (C═O)

νₘₐₓ (DCM)/cm⁻¹ 3360.1, 2925.7, 1598.6, 1506.7, 1275.3, 1026.7

HRMS: calculated 369.1338, found 369.1324, molecular formula (C₂₁H₂₁O₆).

5 ¹H NMR (CDCl₃, 400 MHz) δ_{H} ppm: 3.60 (2H, m, CH₂), 3.66 (3H, s, OMe), 3.95 (3H, s, OMe), 3.97 (3H, s, OMe), 3.99 (3H, s OMe), 4.63 (1H, dd, J=3.0 Hz, 9.0 Hz, CHBr), 5.67 (1H, s, br, OH), 6.39 (1H, s, C═CH), 6.45 (1H, s, ArH {A-ring}), 6.89 (3H, m, 3×ArH {C-ring})

¹³C NMR (CDCl₃, 400 MHz) δ_{c} ppm: 29.26 (HH₂), 52.93 (HHBr), 55.53 (OMe), 55.57 (OMe), 60.43 (OMe), 60.96 (OMe), 109.77 (ArCH), 110.90 (ArCH), 114.95 (ArCH), 120.85 (ArCH), 123.97 (ArC), 124.81 (C═CH), 132.14 (ArC), 134.63 (ArC), 142.80 (ArC), 144.83 (ArC), 147.13 (ArC), 150.71 (ArC), 151.48 (ArC), 196.51 (C═O)

νₘₐₓ (DCM)/cm⁻¹ 3533.7, 2937.8, 1510.3, 1266.1, 737.8

### Synthesis of 5-(3-hydroxy-4-methoxyphenyl)-1,2,3-trimethoxy-6,7,8,9-tetrahydro-5H-benzo[a]cyclohepten-7-one 7

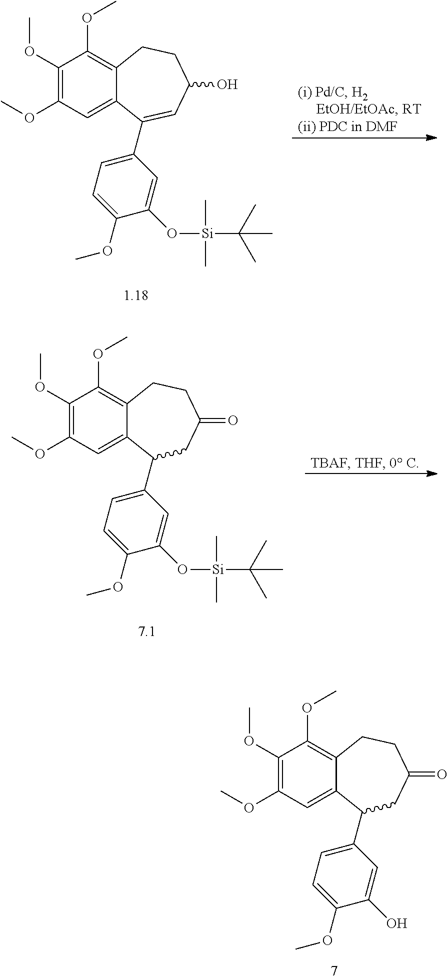

To a solution of 1.18 (0.10 g 0.20 mmol) in ethanol/ethyl acetate (1:1, 4 mL) was added 10% Pd/C (0.1 g). The reaction mixture was stirred under a hydrogen atmosphere for 48 h. On completion, the reaction was filtered and the filtrate was concentrated to afford as an oil. This was re-dissolved in DMF (1 mL) and PDC (0.077 g, 0.20 mmol) was added at 0° C. After 24 h, the reaction was quenched by the addition of water (5 mL) and the product was extracted with diethyl ether (5×5 mL). The organic fractions were collected, dried under sodium sulphate and filtered before the filtrate was concentrated in vacuo to afford 7.1 as an oil. This was re-dissolved in THF (0.5 mL) and 1M TBAF (0.1 mL, 0.10 mmol) was added drop-wise at 0° C. After 2 h, the reaction was quenched by the addition of water (5 mL) and the product was extracted with diethyl ether (3×5 mL). The organic fractions were collected, dried under sodium sulphate and filtered before the filtrate was concentrated in vacuo. The resulting residue was purified by flash column chromatography (stationary phase; silica gel 230-400 mesh, mobile phase; 1:1 hexane/ethyl acetate). All homogenous fractions were collected and the solvent was evaporated off to afford 7 as a white solid (0.036 g, 49%). M.pt. 124-126° C. νₘₐₓ (KBr)/cm⁻¹ 3402.1, 2936.7, 1701.6, 1593.2, 1511.8, 1123.3. GCMS m/z (%) 371 (M⁺+1, 85), 370 (100), 328 (16). ¹H NMR (CDCl₃, 400 MHz) δ_{H} ppm 2.57 (2H, m, CH₂), 2.95 (4H, m, 2×CH₂), 3.74 (3H, s, OMe), 3.86 (3H, s, OMe), 3.90 (6H, s, 2×OMe), 4.33 (1H, dd, J 3.4, 8.0 Hz, CHOH), 5.62 (1H, br, OH), 6.36 (1H, s, ArH), 6.69 (2H, m, 2×ArH), 6.81 (1H, d, J 8.0 Hz, ArH). ¹³C NMR δ_{c} ppm 19.32 (CH₂), 43.75 (CH₂), 45.46 (ArCHAr), 48.67 (CH₂), 55.49 (2×OMe), 60.34 (OMe), 60.89 (OMe), 109.12 (ArCH), 110.13 (ArCH), 113.63 (ArCH), 118.60 (ArCH), 125.64 (qC) 135.53 (qC), 137.77 (qC), 144.82 (qC), 145.21 (qC), 150.98 (qC), 151.57 (qC), 210.28 (C═O).

### Synthesis of (Z)-8,9-dihydro-1,2,3-trimethoxy-5-(naphthalen-3-yl)benzo[7]annulen-7-one 8

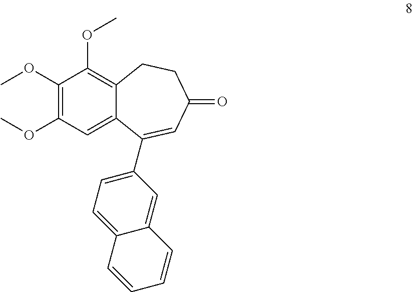 [SMILES: COc1cc2c(c(OC)c1OC)CCC(=O)C=C2c1ccc2ccccc2c1]

### Step 1

### Synthesis of (Z)-6,7-dihydro-2,3,4-trimethoxy-9-(naphthalen-3-yl)-5H-benzo[7]annulen-7-ol 8.1

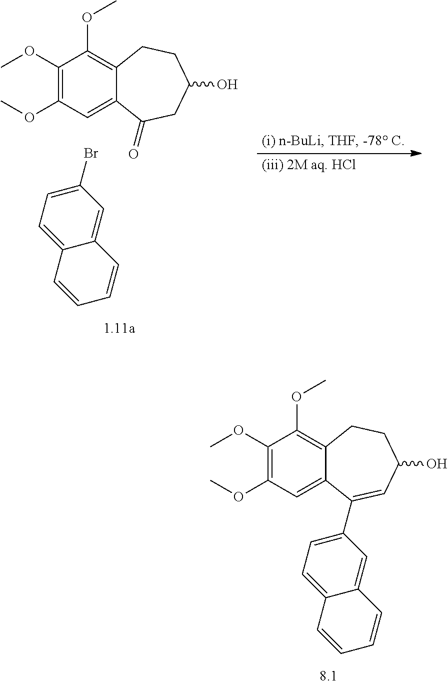

To a stirred solution of 2-bromonaphthalene (5.20 g, 2.5 mmol) dissolved in anhydrous THF (7 mL) was added 2.5M n-BuLi (1.5 mL, 3.8 mmol) drop-wise at −78° C. under anhydrous conditions. After 20 min whilst maintaining the temperature at −78° C., a solution of 1.11a (220 mg, 0.83 mmol) in anhydrous THF (6 mL) was added to the reaction. After 2 h the temperature was allowed to increase to 0° C. and was maintained at this temperature for twelve h. The reaction was quenched by the addition of 2M aq. HCl (1×30 mL). The product was extracted with diethyl ether (3×20 mL). The organic fractions were combined, dried over MgSO₄, filtered and concentrated in vacuo. The resulting residue was purified by flash column chromatography (stationary phase; silica gel 230-400 mesh, mobile phase; 3:1, hexane/ethyl acetate). All homogenous fractions were collected and the solvent was evaporated to afford the product 8.1 as a white solid (90 mg, 29%).

¹H NMR (CDCl₃, 400 MHz) δ_{H} ppm: 2.25 (2H, m, CH₂), 2.55 (2H, m, CH₂), 3.11 (1H, m, CHOH), 3.62 (3H, s, OMe), 3.96 (6H, s, 2×OMe), 4.29 (1H, m, OH), 6.39 (1H, s, ArH {A-ring}), 6.53 (1H, d, J=5.0 Hz, C═CH), 7.48 (3H, d, J=7.0 Hz ArH{naph}), 7.80 (4H, m, ArH{naph})

¹³C NMR (CDCl₃, 400 MHz) δ_{c} ppm: 21.44 (CH₂), 42.95 (CH₂), 55.51 (OMe), 60.47 (OMe), 61.48 (OMe), 69.48 (CHOH), 108.29 (ArCH), 126.18 (2×ArCH), 126.89 (ArCH), 127.52 (ArCH), 127.79 (ArCH), 128.09 (2×ArCH), 132.37 (ArC), 132.91 (ArC), 133.24 (C═CH), 134.67 (ArC), 137.98 (ArC), 138.70 (ArC), 141.10 (ArC), 150.42 (ArC), 150.84 (ArC)

νₘₐₓ (KBr)/cm⁻¹ 3394.2, 2932.7, 1488.5, 1111.5

Melting point: 44-49° C.

### Step 2

### Synthesis of (Z)-8,9-dihydro-1,2,3-trimethoxy-5-(naphthalen-3-yl)benzo[7]annulen-7-one 8

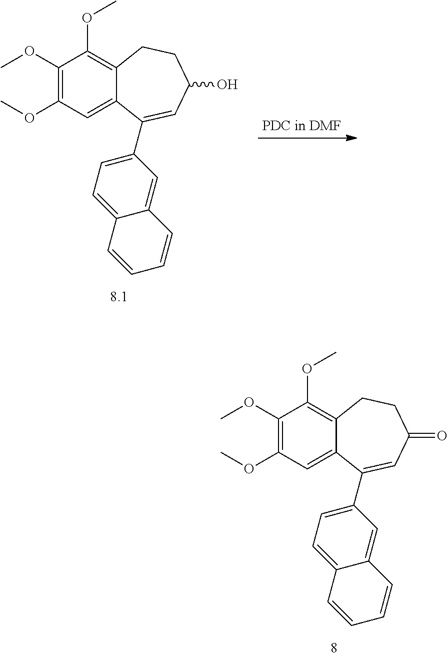

To a stirred solution of 8.1 (50 mg, 0.13 mmol) dissolved in DMF (1 mL) was added pyridinium dichromate (100 mg, 0.27 mmol) at room temperature. The progress of the reaction was monitored by TLC and after 2 h the reaction was quenched by the addition of water (1×50 mL). The product was extracted with diethyl ether (3×30 mL). The organic fractions were combined, dried over MgSO₄, filtered and concentrated in vacuo. The resulting residue was purified by flash column chromatography (stationary phase; silica gel 230-400 mesh, mobile phase; 3:1, hexane/ethyl acetate). All homogenous fractions were collected and the solvent was evaporated to afford 8 as a white solid (30 mg, 60%).

¹H NMR (CDCl₃, 400 MHz) δ_{H} ppm: 2.79 (2H, m, CH₂), 3.23 (2H, t, J=6.0 Hz, CH₂), 3.54 (3H, s, OMe), 3.95 (3H, s, OMe), 3.98 (3H, s, OMe), 6.34 (1H, s, ArH {A-ring}), 6.56 (1H, s, C═CH), 7.38 (1H, dd, J=1.5 Hz, 8.5 Hz, ArH{naph}), 7.56 (2H, q, J=3.0 Hz, ArH{naph}), 7.89 (4H, m, ArH{naph})

¹³C NMR (CDCl₃, 400 MHz) δ_{c} ppm: 19.81 (HH₂), 45.14 (HH₂), 55.56 (OMe), 60.51 (OMe), 61.01 (OMe), 111.36 (ArCH), 126.18 (2×ArCH), 126.34 (ArCH), 126.39 (ArCH), 127.24 (ArCH), 127.30 (2×ArCH), 127.94 (C═CH), 129.12 (ArC), 131.95 (ArC), 132.62 (ArC), 132.94 (ArC), 139.73 (ArC), 142.93 (ArC), 149.69 (ArC), 150.78 (ArC), 151.61 (ArC), 203.53 (C═O)

νₘₐₓ (KBr)/cm⁻¹ 3392.0, 2935.1, 1654.9, 1492.5, 1115.3

Melting point: 105-108° C.

HRMS: calculated 397.1416, found 397.1407, elemental composition (C₂₄H₂₂O₄Na).

### Synthesis of (5Z,8Z)-1,2,3-trimethoxy-5-(naphthalen-3-yl)-7H-benzo[7]annulen-7-one 9 and (5Z,8E)-8-bromo-1,2,3-trimethoxy-5-(naphthalen-3-yl)-7H-benzo[7]annulen-7-one

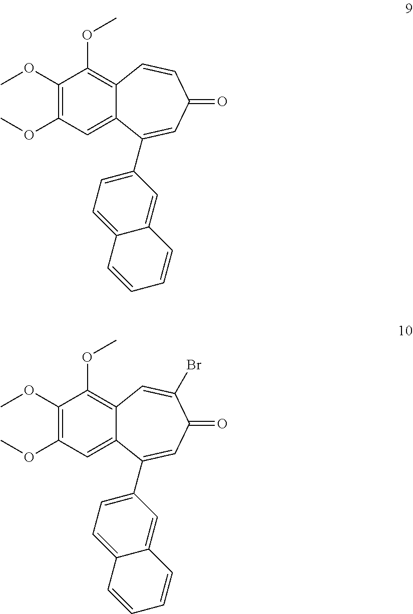 [SMILES: COc1cc2c(-c3ccc4ccccc4c3)cc(=O)ccc2c(OC)c1OC ; COc1cc2c(-c3ccc4ccccc4c3)cc(=O)c(Br)cc2c(OC)c1OC]

### Bromination of 8 with 5,5-dibromo-2,2-dimethyl-4,6-dioxo-1,3-dioxane, followed by Treatment of the Resulting Mixture with Sodium Azide

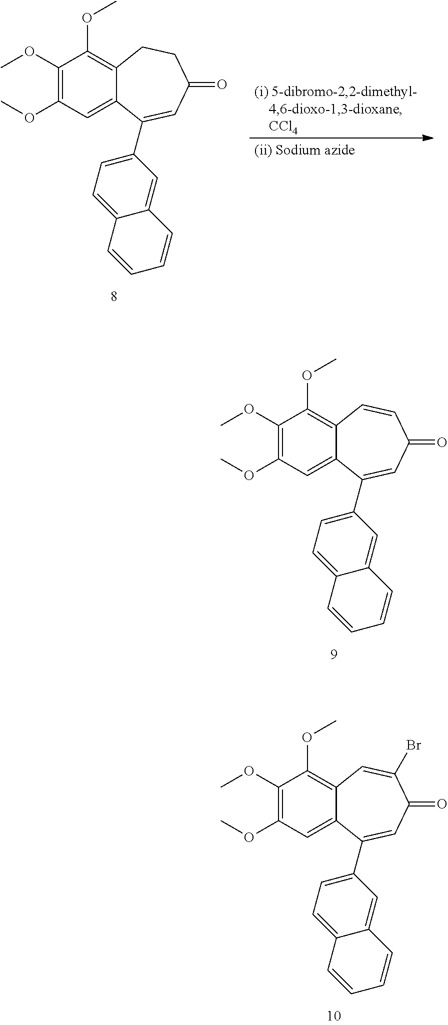

To a stirred solution of 8 (20 mg, 0.05 mmol) in CCl₄ (1 mL) was added 5,5-dibromo-2,2-dimethyl-4,6-dioxo-1,3-dioxane (8 mg, 0.025 mmol). The resulting mixture was refluxed for 2 h. The reaction was quenched by the addition of 5% aq. NaHCO₃ (40 mL) and the product was extracted with diethyl ether (2×40 mL). The combined organic extracts were washed with sat. aq. NaCl (1×50 mL). The organic fraction was dried over MgSO₄, filtered and concentrated under vacuum to afford a yellow oil. The resulting residue was not purified and was used within 2 h of preparation. The residue was dissolved in DMF (2 mL) and NaN₃ (33 mg, 0.50 mmol) was added to the stirred solution. The reaction was left stirring overnight and was quenched by the addition of water (1×20 mL). The product was extracted with diethyl ether (3×20 mL). The combined organic extracts were dried over MgSO₄, filtered and concentrated to an oil in vacuo. The products were purified by either flash column chromatography (stationary phase; silica gel 230-400 mesh, mobile phase; 2:1, hexane/ethyl acetate).

9 ¹H NMR (CDCl₃, 400 MHz) δ_{H} ppm: 3.51 (3H, s, OMe), 4.00 (3H, s, OMe), 4.02 (3H, s, OMe), 6.74 (1H, s, ArH {A-ring}), 6.87 (1H, dd, J=3.0 Hz, 13.0 Hz, CH═CHCO), 7.00 (1H, d, J=3.0 Hz, C═CHCO), 7.42 (1H, dd, J=2.0 Hz, 8.0 Hz, ArH {naph}), 7.59 (2H, dd, J=3.0 Hz, 6.0 Hz, 2×ArH{naph}), 7.92 (4H, m, 4×ArH{naph}), 8.17 (1H, d, J=13.0 Hz, CH═CHCO)

¹³C NMR (CDCl₃, 400 MHz) δ_{c} ppm: 55.21 (OMe), 60.62 (OMe), 61.50 (OMe), 111.66 (ArCH), 125.01 (ArCH), 126.21 (ArCH), 126.31 (ArCH), 126.50 (ArCH), 127.29 (ArCH), 127.36 (ArCH), 127.41 (ArCH), 127.73 (ArCH), 132.24 (ArCH), 132.31 (ArC), 132.81 (ArC), 132.97 (ArCH), 136.92 (ArC), 140.81 (ArC), 150.44 (ArC), 152.56 (ArC), 153.41 (ArC), 187.70 (C═O)

νₘₐₓ (DCM)/cm⁻¹ 3389.9, 2917.3, 1732.6, 1363.1, 1117.6

10 ¹H NMR (CDCl₃, 400 MHz) δ_{H} ppm: 3.50 (3H, s, OMe), 4.01 (3H, s, OMe), 4.02 (3H, s, OMe), 6.74 (1H, s, ArH {A-ring}), 7.27 (1H, s, CH═CBr), 7.45 (1H, dd, J=1.5 Hz, 8.5 Hz, ArH{naph}), 7.58 (1H, q, J=3.5 Hz, 6.0 Hz, ArH{naph}), 7.88 (1H, s, C═CHCO), 7.92 (5H, m, 5×ArH{naph})

¹³C NMR (CDCl₃, 400 MHz) δ_{c} ppm: 55.07 (OMe), 60.59 (OMe), 61.13 (OMe), 107.13 (C═HHCO), 110.03 (ArCH), 126.03 (ArCH), 126.20 (ArCH), 126.75 (ArCH), 127.15 (ArCH), 127.29 (ArCH), 127.36 (ArCH), 127.71 (ArCH), 132.20 (C═HHCO), 132.29 (ArC), 132.82 (ArC), 141.58 (ArC), 146.11 (ArC), 150.32 (ArC), 150.56 (ArC) 181.70 (C═O)

νₘₐₓ (DCM)/cm⁻¹ 3381.7, 2917.3, 1714.6, 1463.7, 1265.4, 737.8

Formation of compound 11.

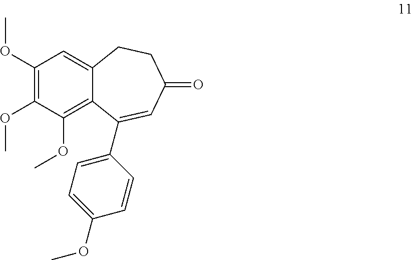 [SMILES: COc1ccc(C2=CC(=O)CCc3cc(OC)c(OC)c(OC)c32)cc1]

### 1^{st} Step

### Synthesis of Intermediate, 3,4,5-trimethoxybenzyl alcohol 11.2

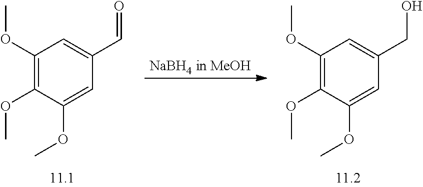

To a stirred solution of 3,4,5-trimethoxybenzylaldehyde (5.00 g, 25.5 mmol) in ethanol (50 mL) was added sodium borohydride (1.13 g, 30.0 mmol) at 0° C. After 1 hour, the solvent was removed under reduced pressure, washed with water (30 mL) and the product was extracted with diethyl ether (3×30 mL). The combined ether extracts were dried over sodium sulphate, filtered and the filtrate was evaporated. The resulting residue was purified by flash column chromatography (stationary phase; silica gel 230-400 mesh, mobile phase; 1:1 hexane/ethyl acetate). All homogenous fractions were collected, reduced in volume to afford 11.2 as an oil (4.85 g, 96%). ¹H NMR (CDCl₃, 400 MHz) δ_{H} ppm 3.85 (3H, s, OMe), 3.86 (3H, s, OMe), 3.94 (3H, s, OMe), 4.60 (2H, s, CH₂), 6.63 (1H, d, J 8.6 Hz, ArH), 6.97 (1H, d, J 8.6 Hz, ArH). ¹³C NMR δ_{c} ppm 55.55 (OMe), 60.27 (OMe), 60.69 (OMe), 60.82 (CH₂), 106.69 (ArH), 122.86 (ArH), 126.50 (qC), 141.62 (qC), 151.37 (qC), 153.16 (qC).

### 2^{nd} Step

### Synthesis of Intermediate, 3,4,5-trimethoxybenzyl bromide 11.3

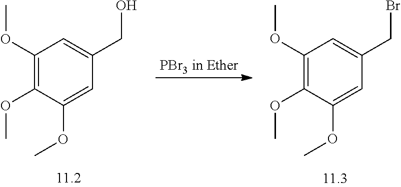

To a stirred solution of 11.2 (4.50 g, 22.7 mmol) in diethyl ether (50 mL) was added PBr₃ (5.34 mL, 34.0 mmol) drop-wise at −20° C. After 2 h, the reaction was quenched with ice-water (50 mL) and the product was extracted with diethyl ether (5×25 mL), washed with 5% aq. NaHCO₃, dried over sodium sulphate and filtered. The filtrate was evaporated and the residue was dried in vacuo for several h to yield 11.3, a white solid (4.68 g, 79%). GCMS m/z (%) 260 (M⁺, 8), 181 (100). ¹H NMR (CDCl₃, 400 MHz) δ_{H} ppm 3.69 (3H, s, OMe), 3.76 (6H, s, 2×OMe), 4.45 (2H, s, CH₂Br), 6.64 (2H, s, ArH). ¹³C NMR δ_{c} ppm 34.92 (CH₂Br), 55.85 (2×OMe), 61.05 (OMe), 106.11 (2×ArCH), 132.68 (qC), 137.81 (qC), 154.82 (2×qC).

### 3^{rd} Step

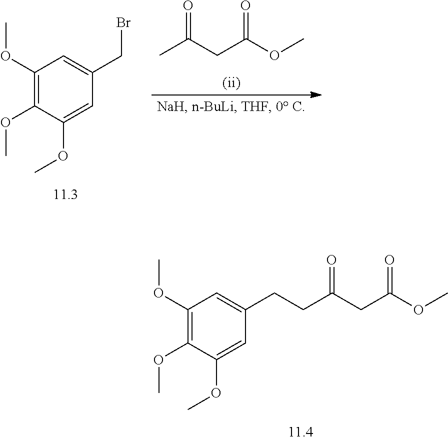

To a stirred solution of anhydrous THF (10 mL) was added NaH (0.506 g, 21.08 mmol) at 0° C. To this suspension was added methyl acetoacetate (2.44 g, 21.08 mmol) slowly over 10 min. When the addition was complete 1.6M n-BuLi (13.17 mL, 21.08 mmol) was added by syringe over a 10 minute period at 0° C. The reaction was allowed to stir for 30 min, after which time, the bromide 11.3 (5.0 g, 19.15 mmol), dissolved in dry THF (10 mL) was added drop-wise. After 2.5 h, the reaction was quenched by the addition of sat. aq. NH₄Cl (25 mL) and the product was extracted with diethyl ether (3×25 mL). The combined ether extracts were dried over sodium sulphate, filtered and the filtrate was evaporated. The resulting residue was purified flash column chromatography (stationary phase; silica gel 230-400 mesh, mobile phase; 4:1 hexane/ethyl acetate). All homogenous fractions were collected and reduced in volume to afford 11.4 as a yellow oil (3.91 g, 69%).

### 4^{th} Step

### Synthesis of Intermediate, methyl 3-hydroxy-5-(3,4,5-trimethoxyphenyl)pentanoate 11.5

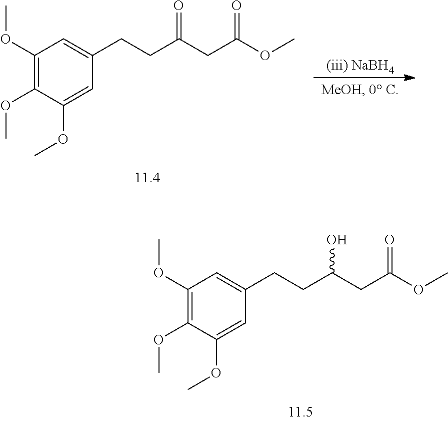 [SMILES: COC(=O)CC(=O)CCc1cc(OC)c(OC)c(OC)c1 ; COC(=O)CC(O)CCc1cc(OC)c(OC)c(OC)c1]

To a stirred solution of 11.4 (0.5 g, 1.68 mmol) in methanol (6.5 mL) was added NaBH₄ (0.02 g, 0.52 mmol) at 0° C. After 2 h, the reaction was quenched with sat. aq. NaCl solution (10 mL) and the product was extracted using diethyl ether (3×10 mL). The organic extracts were collected, dried over sodium sulphate and filtered before the product was concentrated in vacuo. The resulting residue was purified by flash column chromatography (stationary phase; silica gel 230-400 mesh, mobile phase; 2:1 hexane/ethyl acetate). All homogenous fractions were collected and reduced in volume to afford 11.5 as a clear oil (0.45 g, 92%). νₘₐₓ (CCl₄)/cm⁻¹ 3501.7, 2943.3, 2840.0, 1734.6, 1126.8. ¹H NMR (CDCl₃, 400 MHz) δ_{H} ppm 1.76 (2H, m, CH₂), 2.50 (2H, m, CH₂), 2.65 (1H, m, CHCH₂), 2.74 (1H, m, CHCH₂), 3.70 (3H, s, COOCH₃), 3.81 (3H, s, OMe), 3.83 (6H, s, 2×OMe), 4.03 (1H, m, CHOH), 6.42 (2H, s, ArH). ¹³C NMR δ_{c} ppm 25.54 (CH₂), 37.76 (CH₂), 40.71 (CH₂), 51.26 (COOCH₃), 55.60 (2×OMe), 60.32 (OMe), 66.78 (CHOH), 104.96 (2×ArCH), 135.77 (qC), 137.02 (qC), 152.69 (qC), 172.81 (C═O).

### 5^{th} Step

### Synthesis of methyl 3-[1-(tert-butyl)-1,1-diphenylsilyl]oxy-5-(3,4,5-trimethoxyphenyl)pentanoate 11.6

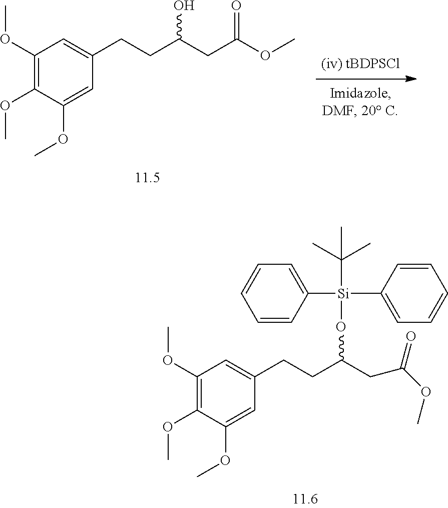 [SMILES: COC(=O)CC(O)CCc1cc(OC)c(OC)c(OC)c1 ; COC(=O)CC(CCc1cc(OC)c(OC)c(OC)c1)O[Si](c1ccccc1)(c1ccccc1)C(C)(C)C]

To a stirred solution of 11.5 (0.99 g, 3.32 mmol) in DMF (5 mL) was added imidazole (0.35 g, 5.14 mmol) and tert-butyldiphenylsilyl chloride (1.89 g, 6.88 mmol) at room temperature. After 2 h, the reaction was quenched with sat. NaCl (10 mL) and the product extracted with diethyl ether (3×10 mL). The organic extracts were collected, dried over sodium sulphate and filtered before the filtrate was concentrated in vacuo. The resulting residue was purified by flash column chromatography (stationary phase: silica gel 230-400 mesh, mobile phase: hexane/ethyl acetate 6:1), to afford, 11.6, as a clear oil (1.74 g, 98%). νₘₐₓ (CCl₄)/cm⁻¹ 3481.3, 2932.4, 2857.3, 1739.6. ¹H NMR (CDCl₃, 400 MHz) δ_{H} ppm 1.11 (9H, s, C(CH₃)₃) 1.82 (2H, m, CH₂), 2.52 (2H, m, H₂), 2.82 (2H, m, CH₂), 3.72 (3H, s, COOCH₃), 3.81 (3H, s, OMe), 3.84 (3H, s, OMe), 3.86 (3H, s, OMe), 4.19 (1H, m, C_HOH), 6.42 (2H, s, {A-ring}2×ArH), 7.41 (6H, m, 6×ArH), 7.78 (4H, m, 4×ArH). ¹³C NMR δ_{c} ppm 18.54 (C(CH₃)₃), 24.60 (CH₂), 26.12 (C(CH₃)₃), 24.68 (CH₂), 39.53 (CH₂), 51.30 (COOMe) 55.80 (OMe), 60.60 (OMe), 60.68 (OMe), 65.32 (CHOH), 105.10 (2×ArCH), 123.46 (ArCH), 127.44 (ArCH), 127.47 (ArCH), 127.64 (ArCH), 129.56 (ArCH), 134.75 (ArCH), 135.92 (ArCH), 135.56 (ArCH), 141.82 (qC), 151.32 (qC), 151.48 (qC), 172.00 (C═O).

### 6th Step

### Synthesis of Intermediate, 3-[1-(tert-butyl)-1,1-diphenylsilyl]oxy-5-(3,4,5-trimethoxyphenyl)pentanoic acid 11.7

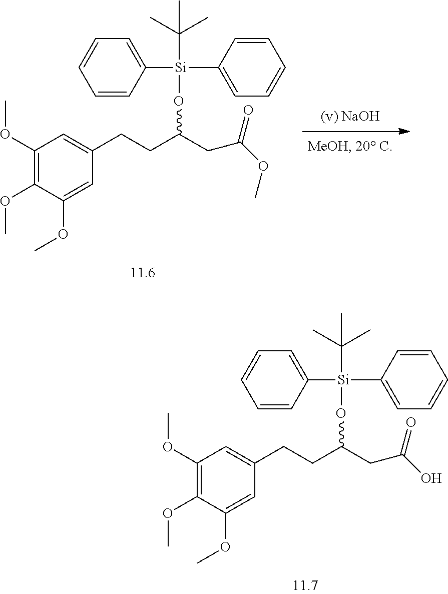 [SMILES: COC(=O)CC(CCc1cc(OC)c(OC)c(OC)c1)O[Si](c1ccccc1)(c1ccccc1)C(C)(C)C ; COc1cc(CCC(CC(=O)O)O[Si](c2ccccc2)(c2ccccc2)C(C)(C)C)cc(OC)c1OC]

To a stirred solution of 11.6 (2.81 g, 5.24 mmol) in methanol (50 mL) was added 1M aq. NaOH (20 mL) at room temperature. After 12 h, the reaction was acidified to pH 2 and the product was extracted with diethyl ether (3×25 mL). The organic fractions were collected and dried over sodium sulphate before being concentrated in vacuo to afford crude 11.7 as a white solid (2.13 g). The product was used directly in the next step without further purification.

### 7^{th} Step

### Synthesis of Intermediate, 7-[1-(tert-butyl)-1,1-diphenylsilyl]oxy-2,3,4-trimethoxy-6,7,8,9-tetrahydro-5H-benzo[a]cyclohepten-5-one 11.8

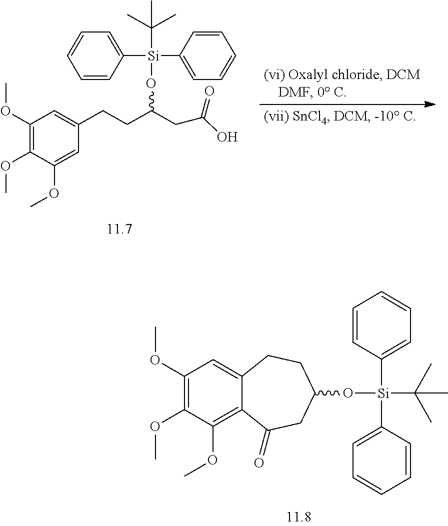 [SMILES: COc1cc(CCC(CC(=O)O)O[Si](c2ccccc2)(c2ccccc2)C(C)(C)C)cc(OC)c1OC ; COc1cc2c(c(OC)c1OC)C(=O)CC(O[Si](c1ccccc1)(c1ccccc1)C(C)(C)C)CC2]

To a stirred solution of the acid 11.7 (1.12 g, 2.14 mmol) in anhydrous DCM (5 mL) was added 2M oxalyl chloride in DCM (2.14 mL, 4.28 mmol) and DMF (1 drop) at −10° C. After 2 h, the excess oxalyl chloride was removed under reduced pressure to afford acylchloride intermediate as an oil. This was re-dissolved in anhydrous DCM (12 mL) and 1.0M SnCl₄ in DCM (0.64 mL, 0.64 mmol) was added at −10° C. After 30 min, the reaction was quenched with sat. aq. NaCl (15 mL) and the product extracted using diethyl ether (3×15 mL). The organic fractions were collected, dried over sodium sulphate and filtered before the filtrate was concentrated in vacuo. The resulting residue was then purified by flash column chromatography (stationary phase; silica gel 230-400 mesh, mobile phase; 2:1 hexane/ethyl acetate). All homogenous fractions were collected and reduced in volume to afford 11.8 as a pale yellow oil (0.76 g, 70%). νₘₐₓ (CCl₄)/cm⁻¹ 2934.9, 1696.3, 1591.5, 1458.9. ¹H NMR (CDCl₃, 400 MHz) δ_{H} ppm 1.08 (9H, s, C(CH₃)₃), 1.96 (2H, m, CH₂), 2.55 (2H, m, CH₂), 2.88 (2H, m, CH₂), 3.83 (3H, s, OMe), 3.85 (3H, s, OMe), 3.88 (3H, s, OMe), 4.19 (1H, m, CHOH), 6.44 (1H, s, ArH), 7.41 (6H, m, 6×ArH), 7.68 (4H, m, 4×ArH). ¹³C NMR δ_{c} ppm 18.74 (C(CH₃)₃), 26.45 (C(CH₃)₃), 29.16 (CH₂), 35.16 (CH₂), 51.90 (CH₂), 55.53 (OMe), 60.38 (OMe), 61.8 (OMe), 68.74 (CHOH), 107.82 (ArCH), 127.48 (ArCH), 127.53 (2×ArCH), 127.62 (2×ArCH), 135.69 (2×ArCH), 135.81 (3×ArCH), 104.76 (qC), 128.38 (qC), 133.18 (qC), 133.56 (qC), 150.94 (qC), 153.79 (2×qC), 200.49 (C═O).

### 8^{th} Step-Deprotection

### Synthesis of 7-hydroxy-2,3,4-trimethoxy-6,7,8,9-tetrahydro-5H-benzo[a]cyclohepten-5-one 11.9

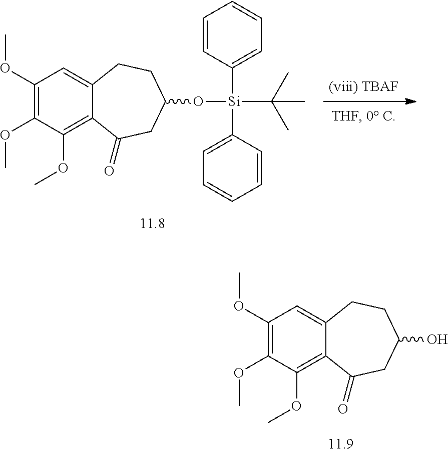 [SMILES: COc1cc2c(c(OC)c1OC)C(=O)CC(O[Si](c1ccccc1)(c1ccccc1)C(C)(C)C)CC2 ; COc1cc2c(c(OC)c1OC)C(=O)CC(O)CC2]

To a stirred solution of 11.8 (0.1 g, 0.19 mmol) in THF (1.0 mL) was added 1M TBAF (0.19 mL, 0.19 mmol) drop-wise at −10° C. After 5 h, the reaction was quenched by the addition of water (5 mL) and the product was extracted with diethyl ether (3×5 mL). The ether extracts were combined, dried over sodium sulphate and filtered before the filtrate was concentrated in vacuo. The residue was then purified by flash column chromatography (stationary phase; silica gel 230-400 mesh, mobile phase; 1:1 hexane/ethyl acetate). All homogenous fractions were collected and reduced in volume to afford 11.9 as a pale yellow solid (0.021 g, 42%). M.pt. 111-112° C. νₘₐₓ (KBr)/cm⁻¹ 3444.0, 2934.5, 1673.6, 1589.3, 1133.0. GCMS m/z (%) 267 (M⁺+1, 100), 266 (82), 248 (35), 239 (31), 181 (75). ¹H NMR (CDCl₃, 400 MHz) δ_{H} ppm 1.92 (1H, m, H-9), 2.18 (1H, m, H-9), 2.65 (1H, m, H-8), 2.89 (1H, m, H-6), 2.97 (1H, m, H-8), 3.02 (1H, m, H-6), 3.86 (3H, s, OMe), 3.89 (6H, s, 2×OMe), 4.20 (1H, m, H-7), 6.47 (1H, s, H-1). ¹³C NMR δ_{c} ppm 29.79 (C-9), 35.17 (C-8), 52.30 (C-6), 55.95 (OMe), 60.81 (OMe), 62.31 (OMe), 68.07 (C-7), 108.44 (C-1), 127.98 (qC), 134.80 (qC), 140.56 (qC), 150.85 (qC), 154.04 (qC), 200.64 (C═O).

### 9th Step

### Synthesis of 1,2,3-trimethoxy-9-(4-methoxyphenyl)-6,7-dihydro-5H-benzo[a]cyclohepten-7-ol 11.10

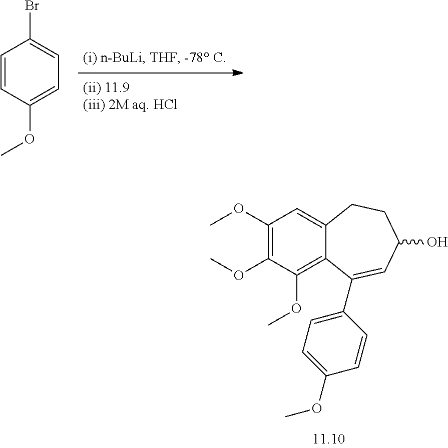

Synthesised using para-bromoanisole (0.07 g, 0.37 mmol) and 11.9 (0.03 g, 0.11 mmol) employing the method described for the preparation of 1.18. Purified by flash column chromatography (stationary phase: silica gel 230-400 mesh, mobile phase: hexane/ethyl acetate 1:1). Afforded 11.10 as a white solid (0.033 g, 83%). M.pt. 41-43° C. νₘₐₓ (KBr)/cm⁻¹ 3422.4, 2936.2, 1594.4, 1246.0, 1117.1. HRMS: found 357.1687 (MH⁺), requires (C₂₁H₂₄O₅) 356.1624. GCMS m/z (%) 338 (M⁺−18, 100), 308 (6), 264 (2). ¹H NMR (CD₃OD, 400 MHz) δ_{H} ppm 2.01 (1H, m, CH), 2.45 (1H, m, CH), 2.55 (1H, m, CH), 2.65 (1H, m, CH), 3.55 (3H, s, OMe), 3.76 (3H, s, OMe), 3.79 (3H, s, OMe), 3.90 (3H, s, OMe), 4.06 (1H, s, CHOH), 6.20 (1H, m, C═CH), 6.77 (1H, s, ArH), 6.85 (2H, m, 2×ArH), 7.12 (2H, m, 2×ArH). ¹³C NMR δ_{c} ppm 29.84 (CH₂), 41.95 (CH₂), 53.85 (OMe), 54.70 (OMe), 58.85 (OMe), 59.39 (OMe), 68.05 (CHOH), 107.05 (ArCH), 112.64 (2×ArCH), 124.34 (qC), 126.46 (2×ArCH), 131.47 (C═CH), 134.10 (qC), 135.68 (qC), 137.21 (qC), 140.32 (qC), 150.52 (qC), 152.46 (qC), 158.47 (qC).

10^{th} Step

### Synthesis of 1,2,3-trimethoxy-9-(4-methoxyphenyl)-6,7-dihydro-5H-benzo[a]cyclohepten-7-one 11

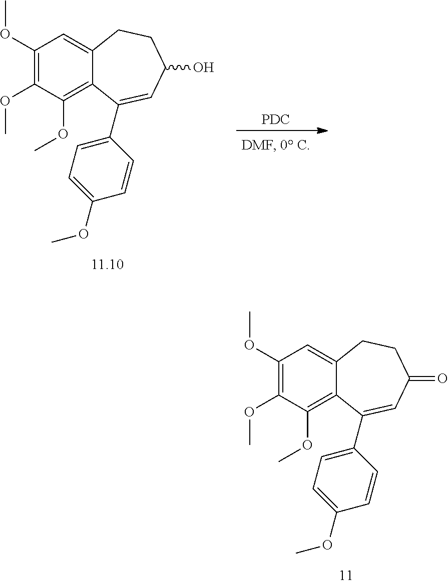 [SMILES: COc1ccc(C2=CC(O)CCc3cc(OC)c(OC)c(OC)c32)cc1 ; "PDC" ; COc1ccc(C2=CC(=O)CCc3cc(OC)c(OC)c(OC)c32)cc1]

To a stirred solution of 11.10 (0.048 g, 0.13 mmol) in DMF (1 mL) was added PDC (0.10 g, 0.27 mmol) portion-wise at 0° C. After 12 h, the reaction was quenched by the addition of water (5 mL) and the product was extracted using diethyl ether (5×5 mL). The organic fractions were collected, dried over sodium sulphate and filtered before being concentrated in vacuo. The resulting residue was purified by flash column chromatography (stationary phase; silica gel 230-400 mesh, mobile phase; 1:1 hexane/ethyl acetate). All homogenous fractions were collected and the solvent was evaporated to afford 11 as a white solid (0.031 g, 68%) as a white solid (0.03 g, 60%). M.pt 36-38° C. HRMS: found 355.1572 (MH⁺), requires (C₂₁H₂₄O₆) 354.1467. GCMS m/z (%) 354 (100), 312 (20), 251 (4), 219 (2). νₘₐₓ (KBr)/cm⁻¹ 2939.8, 1659.8, 1593.6, 1509.8, 1117.5. ¹H NMR (CD₃OD, 400 MHz) δ_{H} ppm 2.67 (2H, m, ArCH₂), 3.25 (3H, s, OMe), 3.32 (2H, m, COCH₂), 3.73 (3H, s, OMe), 3.81 (3H, s, OMe), 3.93 (3H, s, OMe), 6.30 (1H, m, C═CH), 6.83 (1H, s, ArH), 6.91 (2H, d, J 8.5 Hz, 2×ArH), 7.18 (2H, d, J 8.5 Hz, 2×ArH). ¹³C NMR δ_{c} ppm 29.40 (ArCH₂), 46.65 (COCH₂), 53.93 (OMe), 54.70 (OMe), 59.06 (OMe), 59.26 (OMe), 106.24 (ArH), 112.85 (2×ArCH), 126.85 (C═CH), 126.87 (2×ArCH), 129.09 (qC), 135.70 (qC), 137.84 (qC), 140.79 (qC), 149.89 (qC), 152.32 (qC), 153.90 (qC), 159.68 (qC), 205.62 (C═O).

### Formation of 9-(3-hydroxy-4-methoxyphenyl)-1,2,3-trimethoxy-6,7-dihydro-5H-benzo[a]cyclohepten-7-ol 12

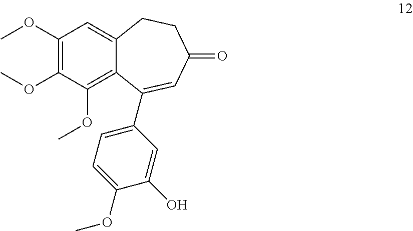 [SMILES: COc1ccc(C2=CC(=O)CCc3cc(OC)c(OC)c(OC)c32)cc1O]

### 1^{st} Step

### Synthesis of Intermediate, 9-(3-[1-(tert-butyl)-1,1-dimethylsilyl]oxy-4-methoxyphenyl)-1,2,3-trimethoxy-6,7-dihydro-5H-benzo[a]cyclohepten-7-ol 12.1

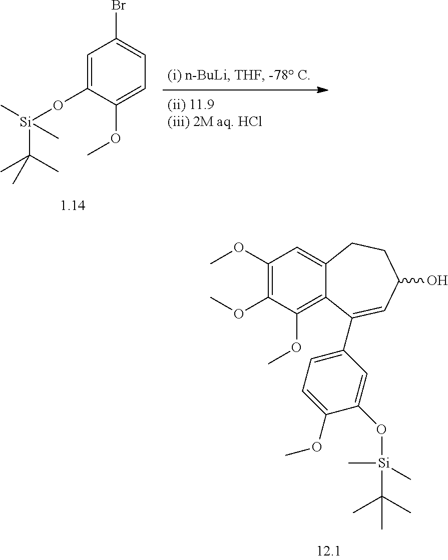 [SMILES: COc1ccc(Br)cc1O[Si](C)(C)C(C)(C)C ; COc1ccc(C2=CC(O)CCc3cc(OC)c(OC)c(OC)c32)cc1O[Si](C)(C)C(C)(C)C]

Synthesised using 11.9 (0.35 g, 1.11 mmol) and 1.14 (0.10 g, 0.37 mmol) employing the method described for the preparation of 11.10. Purified by flash column chromatography (stationary phase: silica gel 230-400 mesh, mobile phase: hexane/ethyl acetate 1:1). Afforded 12.1 as a white solid (0.15 g, 82%). νₘₐₓ (CCl₄)/cm⁻¹ 3402.2, 2932.6, 1507.9, 1117.5. ¹H NMR (CDCl₃, 400 MHz) δ_{H} ppm 0.16 (3H, s, SiCH₃), 0.17 (3H, s, SiCH₃), 1.00 (9H, s, C(CH₃)₃), 2.50 (2H, m, CH₂), 2.71 (2H, m, ArCH₂), 3.40 (3H, s, OMe), 3.80 (6H, s, 2×OMe), 3.92 (3H, s, OMe), 4.23 (1H, m, CHOH), 6.22 (1H, d, J 5.0 Hz, C═CH), 6.62 (1H, s, {A-ring}ArH), 6.75 (2H, m, {C-ring}2×ArH), 6.89 (1H, m, {C-ring}ArH). ¹³C NMR δ_{c} ppm −5.04 (CH₃SiCH₃), 17.99 (C(CH₃)₃), 25.30 (C(CH₃)₃), 30.34 (CH₂), 42.51 (CH₂), 55.08 (OMe), 55.48 (OMe), 59.72 (OMe), 60.25 (OMe), 68.92 (CHOH), 106.75 (ArCH), 111.09 (ArCH), 118.49 (ArCH), 119.25 (ArCH), 124.33 (qC) 131.67 (C═CH), 134.75 (qC), 136.92 (2×qC), 140.41 (qC), 144.30 (qC), 149.77 (qC), 150.86 (qC), 152.40 (qC).

### Step 2

### Synthesis of 9-(3-hydroxy-4-methoxyphenyl)-1,2,3-trimethoxy-6,7-dihydro-5H-benzo[a]cyclohepten-7-one 12

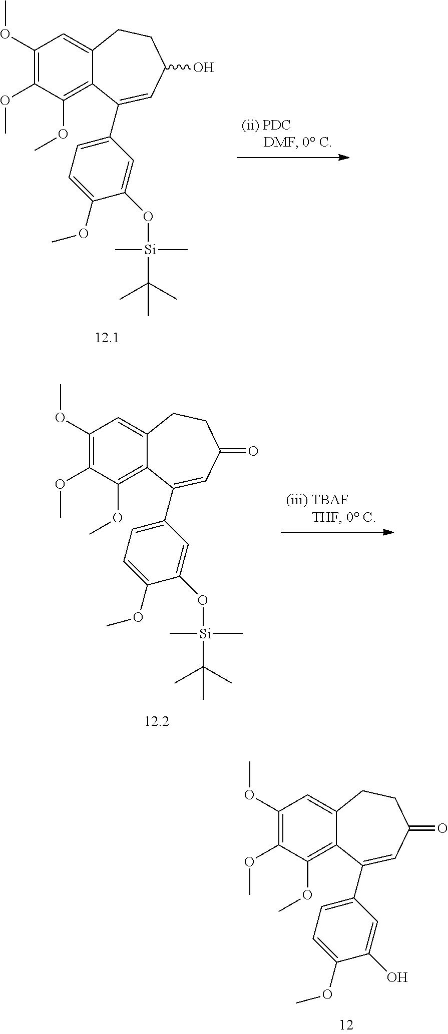 [SMILES: COc1ccc(C2=CC(O)CCc3cc(OC)c(OC)c(OC)c32)cc1O[Si](C)(C)C(C)(C)C ; COc1ccc(C2=CC(=O)CCc3cc(OC)c(OC)c(OC)c32)cc1O[Si](C)(C)C(C)(C)C ; COc1ccc(C2=CC(=O)CCc3cc(OC)c(OC)c(OC)c32)cc1O]

To a stirred solution of 12.1 (0.040 g, 0.08 mmol) in DMF (1 mL) was added PDC (0.061 g, 0.164 mmol) portion-wise at 0° C. After 12 h, the reaction was quenched by the addition of water (5 mL) and the product was then extracted with diethyl ether (5×5 mL). The ether extracts were combined, dried over sodium sulphate and filtered before the filtrate was concentrated in vacuo. The resulting residue was purified by flash column chromatography (stationary phase; silica gel 230-400 mesh, mobile phase; 3:1 hexane/ethyl acetate). All homogenous fractions were collected and the solvent was evaporated off to afford 12.2 as a clear oil (0.02 g, 50%). The enone 12.2 (0.02 g, 0.041 mmol) was subsequently re-dissolved in THF (1 mL) and 1M TBAF (0.08 mL, 0.082 mmol) was added drop-wise at room temperature. After 2 h, the reaction was quenched by the addition of water (5 mL) and the product was extracted using diethyl ether (3×5 mL). The ether extracts were combined, dried over sodium sulphate and filtered before the filtrate was concentrated in vacuo. The resulting residue was purified by flash column chromatography (stationary phase; silica gel 230-400 mesh, mobile phase; 1:1 hexane/ethyl acetate). All homogenous fractions were collected and the solvent was evaporated off to afford 12 as a white solid (0.015 g, 99%). M.pt. 44-46° C. νₘₐₓ (KBr)/cm⁻¹ 3402.7, 2935.2, 1652.2, 1508.7, 1115.8. HRMS: found 371.1465 (MH⁺), requires (C₂₁H₂₂O₆) 370.1416. GCMS m/z (%) 371 (M⁺+1, 100), 370 (96), 328 (22.5). ¹H NMR (CD₃OD, 400 MHz) δ_{H} ppm 2.77 (2H, m, ArCH₂), 3.05 (2H, m, COCH₂), 3.37 (3H, s, OMe), 3.82 (3H, s, OMe), 3.96 (3H, s, OMe), 4.02 (3H, s, OMe), 6.38 (1H, s, C═CH), 6.80 (1H, s, ArH), 6.91 (1H, d, ArH), 7.00 (2H, m, 2×ArH). ¹³C NMR δ_{c} ppm 24.24 (CH₂), 29.37 (CH₂), 54.60 (OMe), 54.70 (OMe), 59.09 (OMe), 59.25 (OMe), 106.12 (ArCH), 110.56 (ArCH), 112.81 (ArCH), 117.27 (ArCH), 126.70 (C═CH), 136.47 (qC), 137.75 (qC), 140.76 (qC), 145.55 (qC), 147.67 (qC), 150.08 (qC), 152.36 (qC), 153.87 (qC), 205.68 (C═O).

### Synthesis of (Z)-5-(3-hydroxy-4-methoxyphenyl)-7,8,9-trimethoxybenzo[b]oxepin-3(2H)-one 13

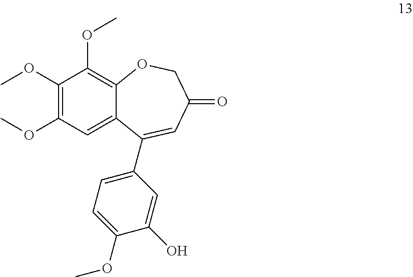 [SMILES: COc1ccc(C2=CC(=O)COc3c2cc(OC)c(OC)c3OC)cc1O]

### Synthesis of Intermediate: 3-hydroxy-7,8,9-trimethoxy-2,3,4,5-tetrahydro-1-benzoxepin-5-one 13.10

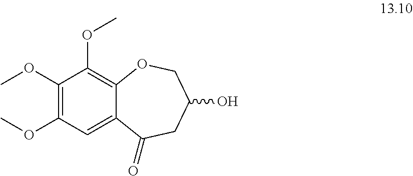 [SMILES: COc1cc2c(c(OC)c1OC)OCC(O)CC2=O]

### 1^{st} Step

### Synthesis of Intermediate, ethyl 2-(2,3,4-trimethoxyphenoxy)acetate 13.2

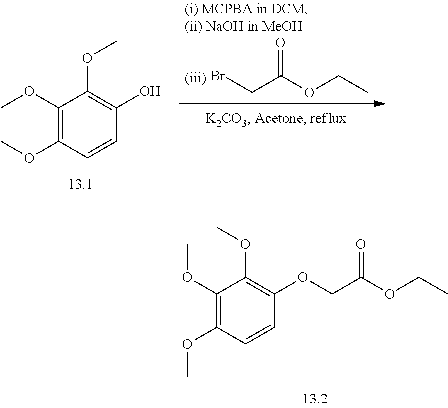

To a stirred solution of 2,3,4-trimethoxybenzaldehyde (3.0 g, 15.3 mmol) in DCM (60 mL) was added a solution of mCPBA (3.26 g, 18.9 mmol) dissolved in DCM (60 mL). After 5 h, the solvent was concentrated to half its volume and filtered to remove the precipitated m-chlorobenzoic acid. The filtrate was then washed with 5% aq. NaHCO₃, water and sat. NaCl. The solvent was subsequently removed under reduced pressure to afford an oily residue. This was re-dissolved in methanol (30 mL) and 2.5M aq. NaOH (25 mL) was added to the solution at 0° C. After 1.5 h, the reaction was acidified with 2M aq. HCl and the product was isolated by extraction with ether (3×20 mL). The combined organic layers were dried under sodium sulphate, filtered and concentrated to an oil. This was purified by flash column chromatography (stationary phase: silica gel; mobile phase: hexane/ethyl acetate 2:1). All homogenous fractions were collected and the solvent was removed in vacuo to afford 13.1 as a yellow solid (2.22 g, 79%). The phenol 13.1 (1.5 g, 8.15 mmol) was re-dissolved in acetone (40 mL) and K₂CO₃ (5.0 g, 36.2 mmol) was subsequently added followed by ethyl bromoacetate (2 mL, 17.3 mmol). The reaction was refluxed for 12 h. On completion, the solvent was concentrated in vacuo and a solution of sat. NaCl (40 mL) was added. The product was extracted using diethyl ether (3×30 mL), dried under sodium sulphate, filtered and concentrated to an oil. It was purified by flash column chromatography (stationary phase: silica gel; mobile phase: hexane/ethyl acetate 5:1). All homogenous fractions were collected and the solvent was removed in vacuo to afford 13.2 as a yellow oil (1.67 g, 76%).

νₘₐₓ (CCl₄)/cm⁻¹ 2984.0, 2939.2, 1748.5, 1591.4, 1120.2. ¹H NMR δ_{H} ppm 1.08 (3H, t, J 7.2 Hz, CH₃), 3.60 (3H, s, OMe), 3.68 (3H, s, OMe), 3.70 (3H, s, OMe), 4.04 (2H, q, J 3.8 Hz, 7.2 Hz, OCH₂CH₃), 4.42 (2H, s, CH₂CO), 6.35 (1H, d, J 9.0 Hz, ArH), 6.40 (1H, d, J 9.0 Hz, ArH). ¹³C NMR δ_{c} ppm 13.40 (CH₂CH₃), 55.53 (OMe), 60.31 (2×OMe), 60.43 (CH₂CH₃), 66.47 (OCH₂), 105.90 (ArCH), 108.94 (ArCH), 142.83 (qC), 143.63 (qC), 145.39 (qC), 148.23 (qC), 168.37 (C═O).

### 2^{nd} Step

### Synthesis of Intermediate, 2,2-dimethyl-5-[-2(2,3,4-trimethoxyphenoxy)acetyl]-1,3-dioxane-4,6-dione 13.4

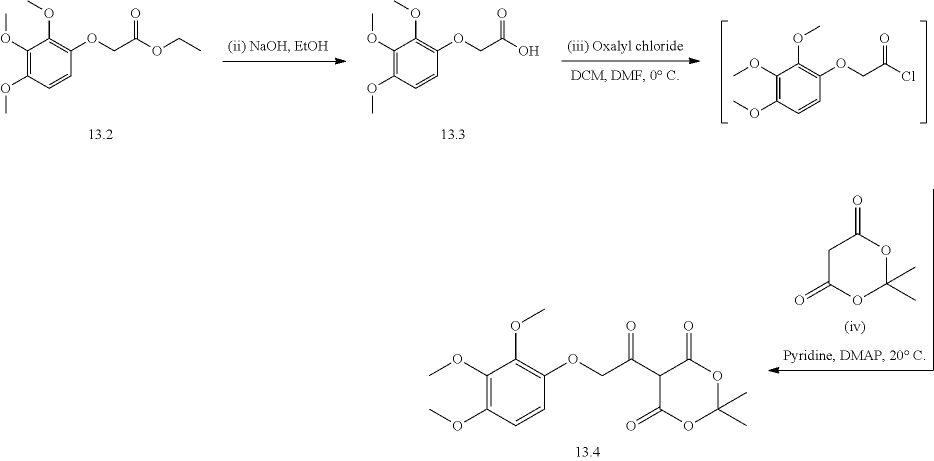

To a stirred solution of ester 13.2 (1.5 g, 5.55 mmol) in ethanol (40 mL) was added 2.5 M aq. NaOH (30 mL) at 25° C. After 3 h, the solvent was removed in vacuo and 2M aq. HCl (40 mL) was added. The product was extracted with diethyl ether (3×30 mL), dried over sodium sulphate, filtered and the solvent was removed under reduced pressure to afford the acid 13.3 as a white solid (1.34 g, 100%). The acid 13.3 (0.88 g, 3.63 mmol) was then re-dissolved in anhydrous DCM (4 mL) and 2M oxalyl chloride solution in DCM (3.63 mL, 7.27 mmol) was added together with DMF (1 drop) under anhydrous conditions at 0° C. for 1 hour. On formation of the acid chloride, the solvent was removed in vacuo to afford a syrupy residue. To this residue was added a solution of Meldrum's acid (0.52 g, 3.61 mmol) dissolved in anhydrous DCM (10 mL) followed by DMAP (0.88 g, 7.21 mmol) at 0° C. for 1 hour. The reaction temperature was then raised to 25° C. and the reaction was allowed to continue for an additional hour. On completion, the solvent was removed in vacuo and 1M aq. HCl (10 mL) was added. The product was extracted with diethyl ether (3×20 mL) and the organic layers were combined, dried under sodium sulphate, filtered and concentrated to afford a yellow solid. This was re-dissolved in DCM (2 mL) and purified by flash column chromatography (stationary phase: silica gel; mobile phase: hexane/ethyl acetate 3:1). All homogenous fractions were collected and the solvent was removed in vacuo to afford 13.4 as a pale-yellow solid (0.83 g, 62%). ¹H NMR δ_{H} ppm 1.78 (6H, s, 2×CH₃), 3.84 (3H, s, OMe), 3.92 (3H, s, OMe), 3.96 (3H, s, OMe), 5.46 (2H, s, 2×H-2), 6.57 (1H, d, J 7.0 Hz, ArH), 6.65 (1H, d, J 7.0 Hz, ArH). ¹³C NMR δ_{c} ppm 26.90 (2×CH₃), 56.33 (OMe), 60.17 (OMe), 61.33 (OMe), 69.48 (C-2), 105.98 (C-2′), 106.49 (ArCH), 109.92 (ArCH), 145.92 (qC), 149.20 (qC), 159.91 (qC), 162.42 (qC), 169.91 (C-4′, C-6′), 192.13 (C-1).

### 3^{rd} Step

### Synthesis of Intermediate, methyl 3-oxo-4-(2,3,4-trimethoxyphenoxy)butanoate 13.5

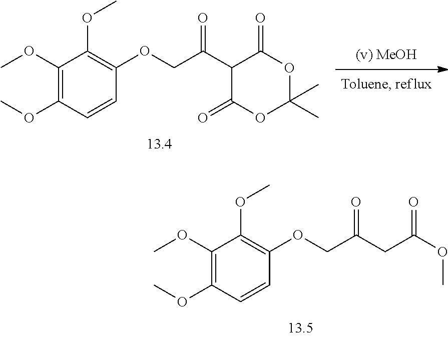

To a stirred solution of 13.4 (0.50 g, 1.36 mmol) in toluene (40 mL) was added methanol (10 mL). The reaction was refluxed for 12 h. On completion, the solvent was removed in vacuo and concentrated to an oil. This oil was purified by flash column chromatography (stationary phase: silica gel; mobile phase: hexane/ethyl acetate 3:1). All homogenous fractions were collected and the solvent was removed in vacuo to afford 13.5 as a clear oil (0.33 g, 82%).

νₘₐₓ (CCl₄)/cm⁻¹ 2931.6, 2826.9, 1753.7, 1738.0, 1491.9, 1115.0. ¹H NMR δ_{H} ppm 3.65 (2H, s, CH₂COOMe), 3.69 (3H, s, COOMe), 3.77 (3H, s, OMe), 3.85 (3H, s, OMe), 3.86 (3H, s, OMe), 4.58 (2H, s, OCH₂CO), 6.51 (2H, s, 2×ArH). ¹³C NMR δ_{c} ppm 45.06 (CH₂CO), 51.81 (COOMe), 55.79 (OMe), 60.58 (OMe), 60.73 (OMe), 74.20 (OCH₂CO), 106.14 (ArCH), 109.14 (ArCH), 143.11 (qC), 143.71 (qC), 145.22 (qC), 148.64 (qC), 166.80 (C═OOMe), 200.04 (C═O).

### 4^{th} step

### Synthesis of Intermediate, methyl 3-hydroxy-4-(2,3,4-trimethoxyphenoxy)butanoate 13.6

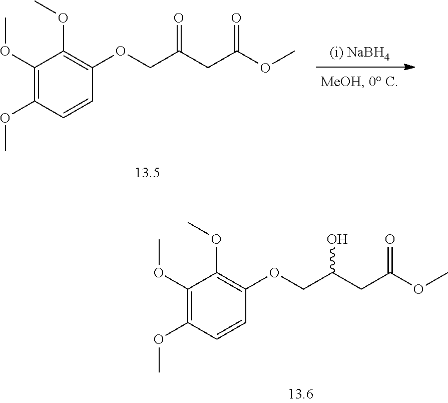 [SMILES: COC(=O)CC(=O)COc1ccc(OC)c(OC)c1OC ; COC(=O)CC(O)COc1ccc(OC)c(OC)c1OC]

To a stirred solution of 13.5 (0.25 g, 0.84 mmol) in methanol (61.6 mL) was added NaBH₄ (0.011 g, 0.29 mmol) at 0° C. After 30 min, the reaction was quenched by the addition of sat. NaCl solution (20 mL) and the product was extracted using diethyl ether (5×25 mL). The ether extracts were combined, dried over sodium sulphate, filtered and the filtrate was concentrated in vacuo to afford an oil. This oil was purified by flash column chromatography (solid phase: silica gel; mobile phase: hexane/ethyl acetate 2:1). All homogenous fractions were collected and the solvent was removed in vacuo to afford 13.6, as an oil (0.18 g, 72%). νₘₐₓ (CCl₄)/cm⁻¹ 3475.1, 2934.2, 2830.2, 1733.8, 1489.4. ¹H NMR δ_{H} ppm 2.66 (2H, m, CH₂COH), 3.42 (1H, br, s, CHOH), 3.71 (3H, s, COOMe), 3.81 (3H, s, OMe), 3.85 (3H, s, OMe), 3.85 (3H, s, OMe), 3.97 (2H, s, OCH₂CHOH), 4.39 (1H, br, s, CHOH), 6.56 (1H, d, J=4.5 Hz, ArH), 6.63 (1H, d, J 4.5 Hz, ArH). ¹³C NMR δ_{c} ppm 37.86 (CH₂COOMe), 51.32 (COOMe), 55.89 (OMe), 60.67 (OMe), 60.86 (OMe), 66.35 (CHOH), 73.31 (OCH₂CHOH), 106.37 (ArCH), 109.66 (ArCH), 142.97 (qC), 143.95 (qC), 146.11 (qC), 148.26 (qC), 171.77 (C═O).

### 5th Step

### Synthesis of methyl 3-[1-(tert-butyl)-1,1-diphenylsilyl]oxy-4-(2,3,4-trimethoxyphenoxy)butanoate 13.7

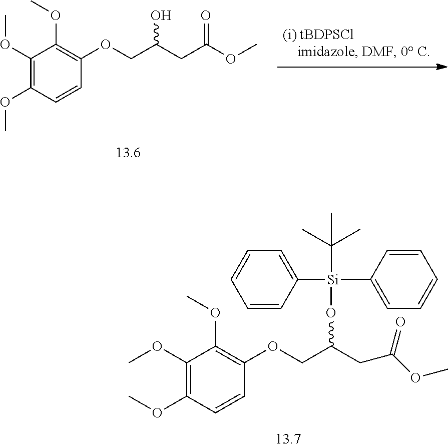

To a stirred solution of 13.6 (0.23 g, 0.77 mmol) in DMF (2 mL) was added tBDPSC1 (0.17 g, 1.15 mmol) followed by imidazole (0.084 g, 1.23 mmol) at 0° C. After 3 h, the reaction was quenched by the addition of sat. aq. NaCl solution (10 mL) and the product was extracted using diethyl ether (3×15 mL). The ether extracts were combined, dried over sodium sulphate, filtered and the solvent was removed in vacuo to afford an oil. This oil was purified by flash column chromatography (stationary phase: silica gel; mobile phase: hexane/ethyl acetate 6:1). All homogenous fractions were collected and the solvent was removed in vacuo to afford 13.7, as an oil (0.32 g, 77%). νₘₐₓ (CCl₄)/cm⁻¹ 2931.6, 2850.3, 1738.0, 1491.9. ¹H NMR δ_{H} ppm 1.09 (9H, s, C(CH₃)₃), 2.77 (2H, 2×dd, J 6.25 Hz, 15.0 Hz, 32.6 Hz, CH₂COOMe), 3.61 (3H, s, COOMe), 3.81 (3H, s, OMe), 3.82 (3H, s, OMe), 3.90 (3H, s, OMe), 3.90 (2H, s, OCH₂CHOSi), 4.55 (1H, m, CHOSi), 6.32 (1H, d, J 9.0 Hz, ArH), 6.47 (1H, d, J 9.0 Hz, ArH), 7.41 (4H, m, 4×ArH), 7.75 (6H, m, 6×ArH). ¹³C NMR δ_{c} ppm 18.80 (C(CH₃)₃), 26.41 (C(CH₃)₃), 39.50 (CH₂), 51.37 (COOMe), 56.35 (OMe), 61.04 (OMe), 61.08 (OMe), 68.86 (CHOSi), 72.03 (OCH₂CHOSi), 106.16 (ArCH), 108.32 (ArCH), 127.16 (2×ArCH), 127.21 (2×ArCH), 129.29 (ArCH), 129.36 (ArCH), 132.93 (qC), 133.22 (qC), 135.40 (2×ArCH), 135.44 (2×ArCH), 143.00 (qC), 143.63 (qC), 146.28 (qC), 147.73 (qC), 171.06 (C═O).

### 6^{th} Step

### Synthesis of Intermediate, 3-[1-(tert-butyl)-1,1-diphenylsilyl]oxy-4-(2,3,4-trimethoxyphenoxy)butanoic acid 13.8

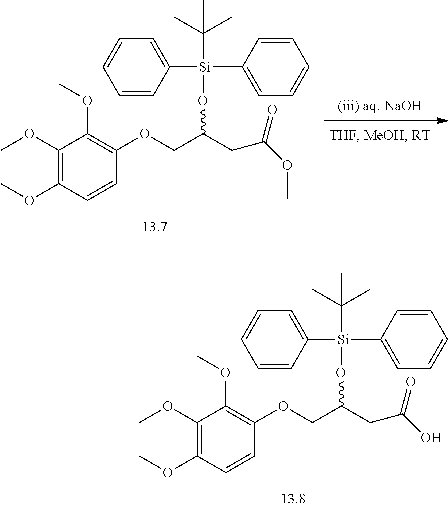 [SMILES: COC(=O)CC(COc1ccc(OC)c(OC)c1OC)O[Si](c1ccccc1)(c1ccccc1)C(C)(C)C ; COc1ccc(OCC(CC(=O)O)O[Si](c2ccccc2)(c2ccccc2)C(C)(C)C)c(OC)c1OC]

To a stirred solution of 13.7 (0.25 g, 0.46 mmol) in methanol (10 mL), THF (7 mL) was added 10% aq. NaOH (10 mL) at room temperature. After 24 h, the reaction was quenched by the addition of 2M aq. HCl (20 mL) and the product was extracted with diethyl ether (3×30 mL). The ether extracts were combined, dried over sodium sulphate, filtered and the filtrate was concentrated in vacuo to afford an oil. This was purified by flash column chromatography (solid phase: silica gel; mobile phase: hexane/ethyl acetate 2:1). All homogenous fractions were collected and the solvent was removed in vacuo to afford 13.8 as a white solid (0.16 g, 66%). νₘₐₓ (CCl₄)/cm⁻¹ 2925.3, 2848.3, 1710.6. ¹H NMR δ_{H} ppm 1.0 (9H, s, C(CH₃)₃), 2.74 (1H, dd, J 6.3 Hz, 15.0 Hz, HCHCOOMe), 2.84 (1H, dd, J 6.3 Hz, 15.0 Hz, HCHCOOMe), 3.810 (3H, s, OMe), 3.818 (3H, s, OMe), 3.98 (3H, s, OMe), 3.90 (2H, s, OCH₂CHOSi), 4.49 (1H, m, CHOSi), 6.30 (1H, d, J 9.0 Hz, ArH), 6.46 (1H, d, J 9.0 Hz, ArH), 7.41 (6H, m, 6×ArH), 7.73 (4H, m, 4×ArH). ¹³C NMR δ_{c} ppm 18.78 (C(CH₃)₃), 26.38 (C(CH₃)₃), 38.86 (CH₂), 55.95 (2×OMe), 60.67 (OMe), 68.18 (CHOSi), 71.86 (CH₂), 106.13 (ArCH), 108.35 (ArCH), 127.17 (2×ArCH), 127.24 (2×ArCH), 129.31 (ArCH), 129.42 (ArCH), 132.64 (qC), 133.10 (qC), 135.36 (2×ArCH), 135.44 (2×ArCH), 142.98 (qC), 143.61 (qC), 143.76 (qC), 146.15 (qC), 147.86 (qC), 175.91 (C═O).

### 7^{th} Step

### Synthesis of Intermediate, 3-[1-(tert-butyl)-1,1-diphenylsilyl]oxy-7,8,9-trimethoxy-2,3,4,5-tetrahydro-1-benzoxepin-5-one 13.9 via 3-[1-(tert-butyl)-1,1-diphenylsilyl]oxy-4-(2,3,4-trimethoxyphenoxy)butanoyl chloride

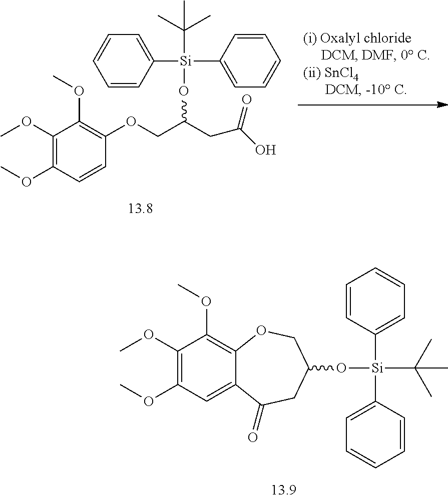 [SMILES: COc1ccc(OCC(CC(=O)O)O[Si](c2ccccc2)(c2ccccc2)C(C)(C)C)c(OC)c1OC ; COc1cc2c(c(OC)c1OC)OCC(O[Si](c1ccccc1)(c1ccccc1)C(C)(C)C)CC2=O]

To a stirred solution of acid 13.8 (0.66 g, 1.26 mmol) in anhydrous DCM (5 mL) was added 2M oxalyl chloride in DCM (1.29 mL, 2.58 mmol) and DMF (1 drop) under anhydrous conditions at 0° C. After 1.5 h, the excess oxalyl chloride was removed under reduced pressure to afford the corresponding acid halide as an oil. This was re-dissolved in anhydrous DCM (12 mL) and a 1.0M SnCl₄ in DCM (0.42 mL, 0.42 mmol) was added at −10° C. After 30 min, the reaction was quenched with sat. NaCl (15 mL) and the product extracted using diethyl ether (3×15 mL). The organic fractions were collected, dried over sodium sulphate and filtered before the filtrate was concentrated in vacuo. The resulting residue was then purified by flash column chromatography (stationary phase; silica gel 230-400 mesh, mobile phase; 2:1 hexane/ethyl acetate). All homogenous fractions were collected and reduced in volume to afford 13.9, as a clear oil (0.50 g, 78%). νₘₐₓ (CCl₄)/cm⁻¹ 2931.6, 2858.3, 1675.2, 1591.4, 1109.7. ¹H NMR δ_{H} ppm 1.06 (9H, s, C(H₃)₃), 3.04 (1H, dd, J 4.5 Hz, 12.5 Hz, HCHCO), 3.11 (1H, dd, J 6.0 Hz, 12.5 Hz, HCHCO), 3.87 (3H, s, OMe), 3.92 (3H, s, OMe), 3.98 (3H, s, OMe), 4.08 (1H, dd, J 4.7 Hz, 12.2 Hz, HCHCHOSi), 4.16 (1H, dd, J 5.7 Hz, 12.2 Hz, HCHCHOSi), 4.48 (1H, m, CHOSi), 7.15 (1H, s, ArH), 7.41 (6H, m, 6×ArH), 7.67 (4H, m, 4×ArH). ¹³C NMR δ_{c} ppm 18.66 (C(CH₃)₃), 26.34 (C(CH₃)₃), 49.35 (CH₂CO), 55.69 (OMe), 60.75 (OMe), 61.27 (OMe), 70.50 (CHOSi), 79.31 (OCH₂CHOSi), 105.00 (ArCH), 123.67 (qC), 127.34 (4×ArCH), 129.49 (ArCH), 129.52 (ArCH), 132.65 (qC), 132.92 (qC), 135.21 (2×ArCH), 135.35 (2×ArCH), 144.23 (qC), 146.88 (qC), 148.29 (qC), 151.67 (qC), 194.97 (C═O).

### 8th Step-Deprotection

### Synthesis of 3-hydroxy-7,8,9-trimethoxy-2,3,4,5-tetrahydro-1-benzoxepin-5-one 13.10

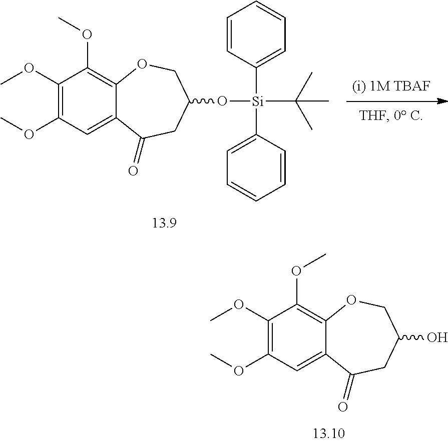

To a stirred solution of 13.9 (0.36 g, 0.71 mmol) in THF (2 mL) was added 1M TBAF (0.78 mL, 0.78 mmol) at 0° C. After 3 h, the reaction was quenched by the addition of sat. NaCl solution (10 mL) and the product was extracted with diethyl ether (3×10 mL). The ether extracts were collected, dried over sodium sulphate, filtered and the solvent was concentrated in vacuo to afford an oil. This was purified by flash column chromatography (stationary phase: silica gel; mobile phase: hexane/ethyl acetate 1:1). All homogenous fractions were collected and the solvent was removed in vacuo to afford 13.10 as a purple solid. (0.10 g, 53%). νₘₐₓ (KBr)/cm⁻¹ 3367.8, 2939.4, 1657.7, 1592.9. ¹H NMR δ_{H} ppm 3.06 (2H, dd, J=5.5 Hz, 12.0 Hz, 2×H-4), 3.80 (3H, s, OMe), 3.86 (3H, s, OMe), 3.92 (3H, s, OMe), 4.22 (2H, m, 2×H-2), 4.48 (1H, m, H-3), 7.04 (1H, s, H-1-6). ¹³C NMR δ_{c} ppm 50.12 (C-4), 55.69 (OMe), 60.71 (OMe), 61.24 (OMe), 69.24 (C-3), 79.92 (C-2), 104.95 (C-6), 122.84 (qC), 143.97 (qC), 146.99 (qC), 148.16 (qC), 152.09 (qC), 195.29 (C═O).

### Formation of 5-(3-hydroxy-4-methoxyphenyl)-7,8,9-trimethoxy-2,3-dihydro-1-benzoxepin-3-one 13

### 1^{st} Step

### Synthesis of Intermediate, 5-(3-[1-(tert-butyl)-1,1-dimethylsilyl]oxy-4-methoxyphenyl)-7,8,9-trimethoxy-2,3-dihydro-1-benzoxepin-3-ol 13.11

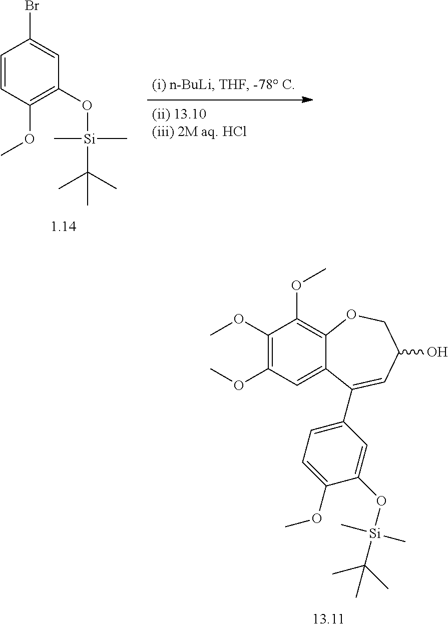 [SMILES: COc1ccc(Br)cc1O[Si](C)(C)C(C)(C)C ; COc1ccc(C2=CC(O)COc3c2cc(OC)c(OC)c3OC)cc1O[Si](C)(C)C(C)(C)C]

To a stirred solution of bromide 1.14 (0.32 g, 1.00 mmol) in anhydrous THF (2 mL) was added 2.5M n-BuLi (0.40 mL, 1.00 mmol) at −78° C. under anhydrous conditions. After 1 hour, the keto-alcohol 13.10 (0.09 g, 0.33 mmol) dissolved in anhydrous THF (2 mL) was added. The reaction was allowed to continue at −78° C. for 8 h. On completion, the reaction was quenched by the addition of 2M aq. HCl (6 mL) and the product was extracted with diethyl ether (3×5 mL). The ether extracts were combined, dried over sodium sulphate, before being concentrated in vacuo. The residue was then purified by flash column chromatography (solid phase; silica gel 230-400 mesh, mobile phase; 1:1 hexane/ethyl acetate). All homogenous fractions were collected and reduced in volume to afford 13.11 as a white solid (0.083 g, 51%). ¹H NMR δ_{H} ppm 0.16 (6H, s, CH₃SiCH₃), 0.99 (9H, s, C(CH₃)₃), 3.57 (3H, s, OMe), 3.84 (3H, s, OMe), 3.92 (3H, s, OMe), 3.97 (3H, s, OMe), 4.10 (2H, m, CHOH), 4.51 (1H, m, OCH₂), 6.10 (1H, d, J 4.5 Hz, C═CH), 6.25 (1H, s, ArH), 6.78 (1H, s, ArH), 6.81 (2H, s, 2×ArH). ¹³C NMR δ_{c} ppm −5.06 (CH₃SiCH₃), 17.95 (C(CH₃)₃), 25.24 (C(CH₃)₃), 55.00 (OMe), 55.61 (OMe), 60.71 (OMe), 61.31 (OMe), 69.83 (CHOH), 78.56 (OCH₂), 109.76 (ArCH), 111.10 (ArCH), 121.34 (ArCH), 122.01 (ArCH), 125.30 (qC), 130.25 (C═CH), 135.71 (qC), 138.37 (qC), 142.22 (qC), 144.07 (qC), 144.74 (qC) 147.49 (qC), 147.79 (qC), 150.02 (qC).

### 2^{nd} Step-Deprotection

### Synthesis of 5-(3-hydroxy-4-methoxyphenyl)-7,8,9-trimethoxy-2,3-dihydro-1-benzoxepin-3-ol 13.12

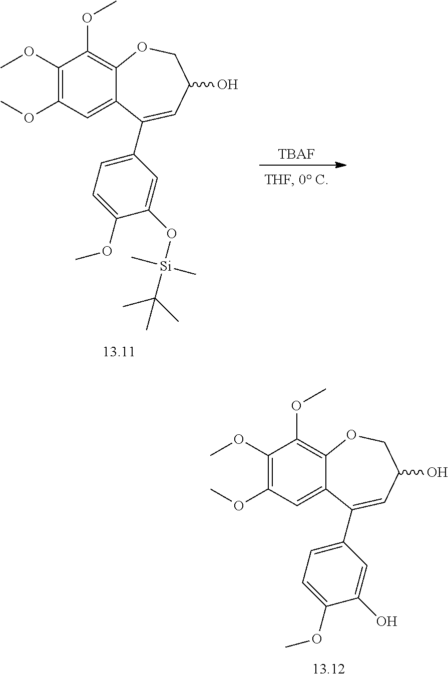 [SMILES: COc1ccc(C2=CC(O)COc3c2cc(OC)c(OC)c3OC)cc1O[Si](C)(C)C(C)(C)C ; "TBAF" ; COc1ccc(C2=CC(O)COc3c2cc(OC)c(OC)c3OC)cc1O]

To a stirred solution of 13.11 (0.017 g, 0.035 mmol) in THF (1.0 mL) was added 1M TBAF (0.035 mL, 0.035 mmol) drop-wise at 0° C. After 2 h, the reaction was quenched by the addition of water (5 mL) and the product was extracted using diethyl ether (3×5 mL). The ether extracts were combined, dried over sodium sulphate and filtered before being concentrated in vacuo. The residue was then purified by flash column chromatography (solid phase; silica gel 230-400 mesh, mobile phase; 1:1 hexane/ethyl acetate). All homogenous fractions were collected and the solvent was evaporated off to afford 13.12 as a white solid (0.012 g, 92%). M.pt. 165-166° C. νₘₐₓ (KBr)/cm⁻¹ 3458.4, 2931.6, 1578.8, 1124.9. HRMS: found 375.1463 (MH⁺), requires (C₂₀H₂₂O₇) 374.1366. GCMS m/z (%) 356 (M⁺−18, 100), 342 (38), 309 (32), 281 (9). ¹H NMR δ_{H} ppm 3.60 (3H, s, OMe), 3.94 (6H, s, 2×OMe), 3.98 (3H, s, OMe), 4.11 (1H, dd, J 2.5 Hz, 9.0 Hz, H-2), 4.47 (1H, q, CHOH), 4.51 (1H, dd, H-2), 5.63 (1H, br, s, OH), 6.14 (1H, d, J 4.5 Hz, C═CH), 6.29 (1H, s, ArH), 6.78 (1H, dd, J 1.5 Hz, 8.0 Hz, H-6′), 6.84 (1H, d, J 8.0 Hz, H-5′), 6.88 (1H, d, J 1.5 Hz, H-2′). ¹³C NMR δ_{c} ppm 55.52 (OMe), 55.76 (OMe), 60.73 (OMe), 61.33 (OME), 69.87 (CHOH), 78.70 (OCH₂), 109.48 (ArCH), 109.75 (ArCH), 115.00 (ArCH), 120.36 (ArCH), 125.21 (qC), 130.42 (C═CH), 136.29 (qC), 138.42 (qC), 142.31 (qC), 144.76 (qC), 144.78 (qC), 145.64 (qC), 147.58 (qC), 147.86 (qC).

### Synthesis of 5-(3-hydroxy-4-methoxyphenyl)-7,8,9-trimethoxy-2,3-dihydro-1-benzoxepin-3-one 13 via the synthesis of 5-(3-[1-(tert-butyl)-1,1-dimethylsilyl]oxy-4-methoxyphenyl)-7,8,9-trimethoxy-2,3-dihydro-1-benzoxepin-3-one 13.13

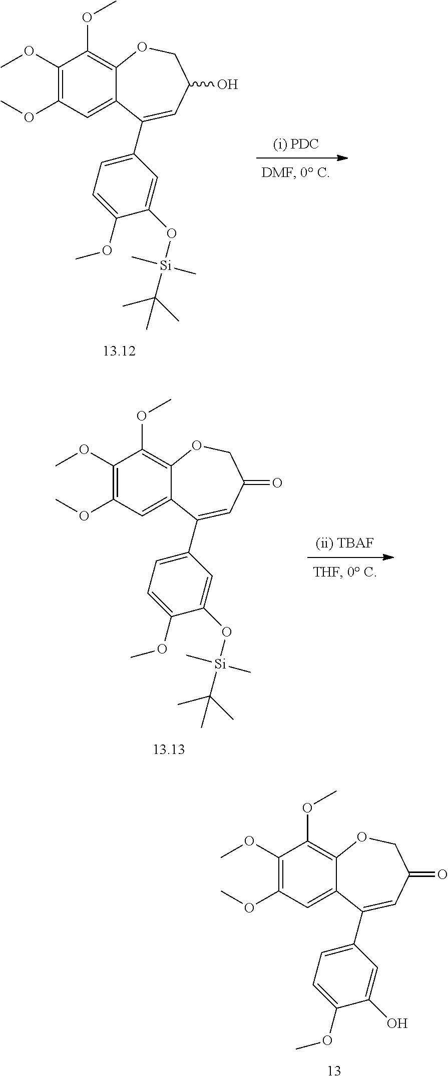 [SMILES: COc1ccc(C2=CC(O)COc3c2cc(OC)c(OC)c3OC)cc1O[Si](C)(C)C(C)(C)C ; "(i) PDC" ; COc1ccc(C2=CC(=O)COc3c2cc(OC)c(OC)c3OC)cc1O[Si](C)(C)C(C)(C)C ; "(ii) TBAF" ; COc1ccc(C2=CC(=O)COc3c2cc(OC)c(OC)c3OC)cc1O]

To a stirred solution of 13.12 (0.043 g, 0.088 mmol) in DMF (1 mL) was added PDC (0.066 g, 0.175 mmol) portion-wise at 0° C. After 12 h the reaction was quenched by the addition of water (5 mL) and the product was extracted with diethyl ether (4×5 mL). The ether extracts were combined, dried over sodium sulphate and filtered before the filtrate was concentrated in vacuo. The residue was purified by flash column chromatography (stationary phase; silica gel 230-400 mesh, mobile phase; 1:1 hexane/ethyl acetate). All homogenous fractions were collected and the solvent was evaporated off to afford 13.13 as a white solid (0.017 g, 55%). A solution of 1M TBAF (0.10 mL, 0.103 mmol) was subsequently added to a stirred solution of 13.13 (0.05 g, 0.103 mmol) in THF (1 mL) at 0° C. After 2 h, the reaction was quenched by the addition of water (5 mL) and the product was extracted with diethyl ether (3×5 mL). The ether extracts were combined, dried over sodium sulphate and filtered before being concentrated in vacuo. The resulting residue was purified by flash column chromatography (stationary phase; silica gel 230-400 mesh, mobile phase; 1:1 hexane/ethyl acetate). All homogenous fractions were collected and the solvent was evaporated off to afford 13 as a yellow solid (0.036 g, 95%). M.pt. 150-152° C. HRMS: found 373.1310 (MH⁺), requires (C₂₀H₂₀O₇) 372.1209. GCMS m/z (%) 372 (100), 329 (25). νₘₐₓ (KBr)/cm⁻¹ 3298.1, 2936.7, 1643.8, 1122.6. ¹H NMR δ_{H} ppm 3.64 (3H, s, OMe), 3.97 (3H, s, OMe), 3.99 (3H, s, OMe), 4.00 (3H, s, OMe), 4.63 (2H, m, 2×H-2), 5.67 (1H, br, s, OH), 6.35 (1H, s, H-4), 6.45 (1H, s, H-6), 6.89 (2H, br, H-5′, H-6′), 6.95 (1H, s, H-2′). ¹³C NMR δ_{c} ppm 55.55 (OMe), 55.85 (OMe), 60.80 (OMe), 61.39 (OMe), 80.68 (C-2), 109.80 (C-6), 110.18 (C-5′), 115.10 (C-2′), 120.91 (C-6′), 125.66 (qC), 127.74 (C-4), 134.42 (qC), 134.62 (qC), 144.93 (qC), 147.10 (qC), 148.73 (qC), 151.32 (qC), 200.03 (C═O).

### Synthesis of 5-(3-hydroxy-4-methoxyphenyl)-3,7,8,9-tetramethoxy-2,3-dihydro-1-benzoxepin-2-one 14

### Bromination of (13.13) with PTAB

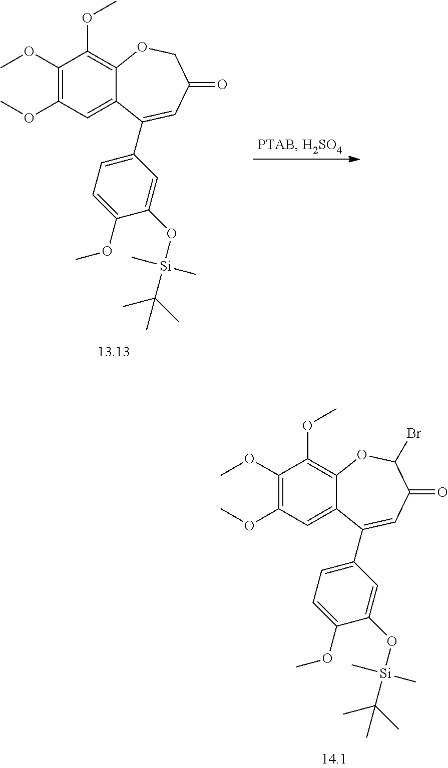

To a stirred solution of 13.13 (30 mg, 0.06 mmol) in ethyl acetate (2 mL) was added H₂SO₄ (0.002 mL) in ethyl acetate (0.02 mL). Phenyltrimethylammonium tribromide (0.03 g, 0.08 mmol) was added to the stirred solution. After 90 min the reaction was quenched by the addition of 5% aq. NaHCO₃ (20 mL) and the product was extracted with ethyl acetate (2×50 mL). The combined organic extracts were washed with sat. aq. NaCl (1×50 mL). The organic fraction was dried over MgSO₄, filtered and concentrated under vacuum. The resulting residue was purified by flash column chromatography (stationary phase; silica gel 230-400 mesh, mobile phase; 3:1, hexane/ethyl acetate). All homogenous fractions were collected and the solvent was evaporated to afford 14.1 as a yellow oil (40 mg, c.100%).

¹H NMR (CDCl₃, 400 MHz) δ_{H} ppm: 0.18 (6H, s, 2×SiCH₃), 1.01 (9H, s, C(CH₃)₃), 3.65 (3H, s, OMe), 3.89 (3H, s, OMe), 4.01 (3H, s, OMe), 4.03 (3H, s, OMe), 6.39 (1H, s, ArH {A-ring}), 6.44 (1H, d, J=1.5 Hz, CHBr), 6.73 (1H, d, J=1.5 Hz, C═CH), 6.87 (1H, m, ArH {C-ring}), 6.90 (1H, s, ArH {C-ring}), 6.96 (1H, dd, J=2.0 Hz, 8.5 Hz, ArH {C-ring})

¹³C NMR (CDCl₃, 400 MHz) δ_{c} ppm: −4.59 (2×SiCH₃), 18.41 (C(CH₃)₃), 25.63 (C(CH₃)₃), 55.39 (OMe), 55.96 (OMe), 61.22 (OMe), 61.72 (OMe), 86.37 (CHBr), 110.26 (ArCH), 111.32 (ArCH), 121.81 (ArCH), 123.18 (ArCH), 125.74 (C═CH), 131.60 (ArC), 133.87 (ArC), 142.11 (ArC), 144.70 (ArC), 144.93 (ArC), 146.21 (ArC), 149.87 (ArC), 152.18 (ArC), 152.84 (ArC), 190.78 (C═O)

νₘₐₓ (DCM)/cm⁻¹ 2933.9, 1726.0, 1512.3, 1130.7, 838.9

### Alternative Synthesis: Bromination of (13.13) with PTAB in THF

### Synthesis of 2-bromo-5-{3-[(tert-butyldimethylsilyl)oxy]-4-methoxyphenyl}-7,8,9-trimethoxy-2,3-dihydro-1-benzoxepin-3-one

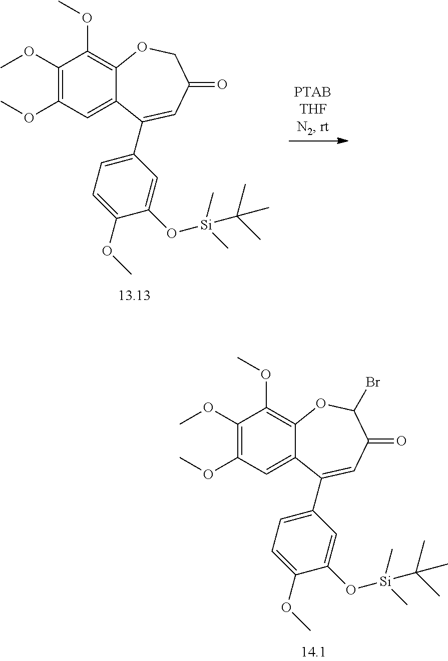 [SMILES: COc1ccc(C2=CC(=O)COc3c2cc(OC)c(OC)c3OC)cc1O[Si](C)(C)C(C)(C)C ; "PTABTHFN2, rt" ; COc1ccc(C2=CC(=O)C(Br)Oc3c2cc(OC)c(OC)c3OC)cc1O[Si](C)(C)C(C)(C)C]

To a solution of 5-{3-[tert-butyldimethylsilyl)oxy]-4-methoxyphenyl}-7,8,9-trimethoxy-2,3-dihydro-1-benzoxepin-3-one (0.23 g, 0.473 mmol) in dry THF (2 mL), at room temperature and under an atmosphere of nitrogen, was added dropwise phenyltrimethylammonium tribromide (0.231 g, 0.6144 mmol) in dry THF (1 mL) and the reaction progress monitored by TLC. After approximately 1 h the reaction was quenched with cold water (50 mL) and extracted with diethyl ether (3×50 mL) before drying with magnesium sulphate and concentration under reduced pressure. The reaction mixture was then purified by column chromatography (3:1 hexane:ethylacetate) to afford 2-bromo-5-{3-[tert-butyldimethylsilyl)oxy]-4-methoxyphenyl}-7,8,9-trimethoxy-2,3-dihydro-1-benzoxepin-3-one 14.1 as a viscous yellow oil (0.214 g, 0.374 mmol, 81%).

¹H NMR (DMF-d7, 400 MHz) δH: 0.19 (6H, s, 2×SiCH₃), 1.01 (9H, s, 3×C(CH₃)₃), 3.70 (3H, s, OCH₃), 3.95 (3H, s, OCH₃), 3.96 (3H, s, OCH₃), 4.00 (3H, s, OCH₃), 6.45 (1H, s, C═CH), 6.53 (1H, s, CHBr), 6.95 (1H, s, ArH), 7.1 (1H, d, ArH, J=8 Hz), 7.17 (1H, s, ArH), 7.19 (1H, d, ArH, J=8 Hz)

¹³C NMR (DMF-d7, 400 MHz) δC: −4.5 (2×SiCH₃), 18.8 (C(CH₃)₃), 25.9 (C(CH₃)₃), 55.9 (OCH₃), 56.4 (OCH₃), 61.3 (OCH₃), 61.8 (OCH₃), 88.0 (CHBr), 111.1 (ArCH), 112.8 (ArCH), 122.1 (ArCH), 124.0 (ArCH), 126.7 (C═CH), 134.2 (ArC), 142.7 (ArC), 145.2 (ArC), 145.7 (ArC), 147.0 (C═C), 150.9 (ArC), 152.8 (ArC), 153.0 (ArC), 152.8 (ArC), 191.4 (C═O)

νₘₐₓ (DCM)/cm⁻¹: 2933.9, 1726.0, 1512.3, 1130.7, 838.9

HRMS m/z 565.1220 (M+H), 587.1041 (M+Na)

### Synthesis of 5-{3-[(tert-butyldimethylsilyl)oxy]-4-methoxyphenyl}-3,7,8,9-tetramethoxy-2,3-dihydro-1-benzoxepin-2-one 13.15 and 5-(3-hydroxy-4-methoxyphenyl)-3,7,8,9-tetramethoxy-2,3-dihydro-1-benzoxepin-2-one 14

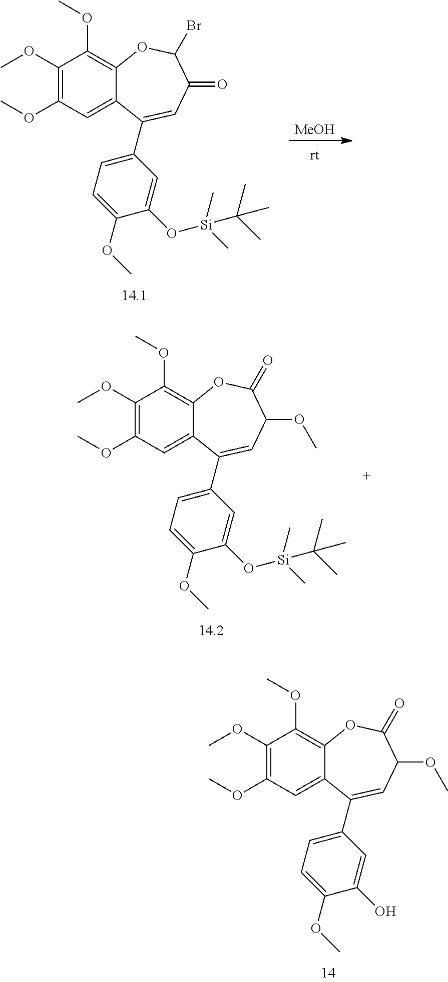

2-bromo-5-{3-[tert-butyldimethylsilyl)oxy]-4-methoxyphenyl}-7,8,9-trimethoxy-2,3-dihydro-1-benzoxepin-3-one 14.1 (0.21 g, 0.371 mmol) was stirred in methanol (5 mL) at room temperature and the reaction monitored by TLC. After a period of approximately 3 h the solvent was removed under reduced pressure. After purification by column chromatography (6-1:1 hexane:ethyl acetate), 5-{3-[tert-butyldimethylsilyl)oxy]-4-methoxyphenyl}-3,7,8,9-tetramethoxy-2,3-dihydro-1-benzoxepin-2-one 14.2 (0.11 g, 0.213 mmol, 57%) and 5-(3-hydroxy-4-methoxyphenyl)-3,7,8,9-tetramethoxy-2,3-dihydro-1-benzoxepin-2-one 14 (0.07 g, 20%) were isolated as viscous oils.

### 5-{3-[(tert-butyldimethylsilyl)oxy]-4-methoxyphenyl}-3,7,8,9-tetramethoxy-2,3-dihydro-1-benzoxepin-2-one 14.2

¹H NMR (CDCl3, 400 MHz) δH: 0.20 (6H, d, 2×SiCH₃), 1.02 (9H, s, C(CH₃)₃), 3.68 (3H, s, OCH₃), 3.81 (3H, s, OCH₃), 3.88 (3H, s, OCH₃), 3.97 (3H, s, OCH₃), 4.05 (3H, s, OCH₃), 5.48 (1H, d, CH(OCH₃), J=5.1 Hz), 5.76 (1H, d, C═CH, J=5.1 Hz), 6.39 (1H, s, ArH), 6.85 (1H, d, ArH, J=2 Hz), 6.90 (1H, s, ArH), 6.92 (1H, d, ArH) ¹³C NMR (CDCl₃, 400 MHz) δC: −4.5 (2×SiCH₃), 18.4 (C(CH₃)₃), 25.7 (C(CH₃)₃), 52.4 (CH(OCH₃), 55.5 (OCH₃), 56.4 (OCH₃), 61.3 (OCH₃), 61.4 (OCH₃), 73.1 (CH(OCH₃)), 105.1 (ArCH), 111.8 (ArCH), 115.9 (C═CH), 117.5 (ArC), 121.2 (ArCH), 122.1 (ArCH), 130.1 (ArC), 137.1 (ArC), 141.3 (ArC), 142.3 (ArC), 143.7 (ArC), 144.8 (ArC), 147.3 (C═C), 151.0 (ArC), 170.3 (C═O)

νₘₐₓ (DCM)/cm⁻¹: 2932.43, 2856.93, 1756.58, 1509.44,

HRMS m/z 539.2076 (M+Na)

### 5-(3-hydroxy-4-methoxyphenyl)-3,7,8,9-tetramethoxy-2,3-dihydro-1-benzoxepin-2-one

14 ¹H NMR (CDCl₃, 400 MHz) δH: 3.69 (3H, s, OCH₃), 3.80 (3H, s, OCH₃), 3.96 (6H, s, 2×OCH₃), 4.05 (3H, s, OCH₃), 5.47 (1H, d, CH(OCH₃), J=5 Hz), 5.67 (1H, br s, OH), 5.78 (1H, s, C═CH, J=5 Hz), 6.42 (1H, s, ArH), 6.88 (2H, m, 2×ArH), 6.96 (1H, d, ArH, J=1.5 Hz).

¹³C NMR (CDCl₃, 400 MHz) δC: 52.4 (OCH₃), 55.9 (OCH₃), 56.5 (OCH₃), 61.3 (OCH₃), 61.4 (OCH₃), 72.9 (CH(OCH₃)), 105.3 (ArCH), 110.4 (ArCH), 114.9 (ArCH), 116.0 (C═CH), 117.3 (ArC), 120.4 (ArCH), 130.7 (ArC), 137.1 (C═C), 141.3 (ArC), 142.3 (ArC), 143.8 (ArC), 145.5 (ArC), 146.6 (ArC), 147.2 (ArC), 170.2 (C═O).

νₘₐₓ (DCM)/cm⁻¹: 3428.98, 2929.41, 1750.40, 1510.69, 1460.35

HRMS: m/z 425.1352 (M+Na)

### Synthesis of 4-(3-hydroxy-4-methoxyphenyl)-6,7,8-trimethoxy-2H-chromen-2-one 15

### Step 1

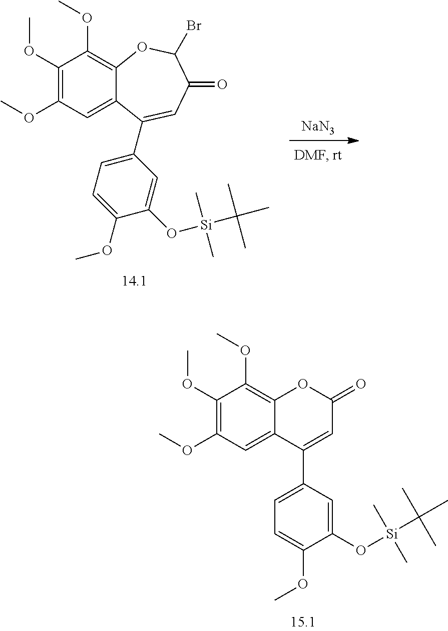 [SMILES: COc1ccc(C2=CC(=O)C(Br)Oc3c2cc(OC)c(OC)c3OC)cc1O[Si](C)(C)C(C)(C)C ; COc1ccc(-c2cc(=O)oc3c(OC)c(OC)c(OC)cc23)cc1O[Si](C)(C)C(C)(C)C ; "NaN3"]

To a stirred solution of the bromide 14.1 (40 mg, 0.07 mmol) in DMF (1 mL) was added NaN₃ (46 mg, 0.70 mmol) at room temperature. The reaction was left stirring overnight and was quenched by the addition of water (1×20 mL). The product was extracted with diethyl ether (3×20 mL). The combined organic extracts were dried over MgSO₄, filtered and concentrated to an oil in vacuo. The resulting residue was purified by flash column chromatography (stationary phase; silica gel 230-400 mesh, mobile phase; 3:1, hexane/ethyl acetate). All homogenous fractions were collected and the solvent was evaporated to afford 15.1 as a yellow solid (20 mg, 61%).

¹H NMR (CDCl₃, 400 MHz) δ_{H} ppm: 0.21 (6H, s, 2×SiCH₃), 1.02 (9H, s, C(CH₃)₃), 3.77 (3H, s, OMe), 3.92 (3H, s, OMe), 4.03 (3H, s, OMe), 4.07 (3H, s, OMe), 6.28 (1H, s, C═CH), 6.78 (1H, s, ArH {A-ring}), 6.98 (3H, m, 3×ArH {C-ring})

¹³C NMR (CDCl₃, 400 MHz) δ_{c} ppm: −4.98 (2×SiCH₃), 17.99 (C(CH₃)₃), 25.20 (C(CH₃)₃), 55.04 (OMe), 55.81 (OMe), 61.07 (OMe), 61.46 (OMe), 102.82 (ArCH), 111.57 (ArCH), 113.01 (C═CH), 114.14 (ArC), 120.46 (ArCH), 121.55 (ArCH), 127.63 (ArC), 140.92 (ArC), 142.97 (ArC), 144.74 (ArC), 145.37 (ArC), 149.10 (ArC), 151.79 (ArC), 154.82 (ArC), 160.36 (C═O)

νₘₐₓ (KBr)/cm^{˜}2916.4, 1725.7, 1260.1, 1091.7

Melting Point: 114-117° C.

HRMS: calculated 473.1996, found 473.2012, elemental composition (C₂₅H₃₃O₇Si).

### Step 2: Deprotection

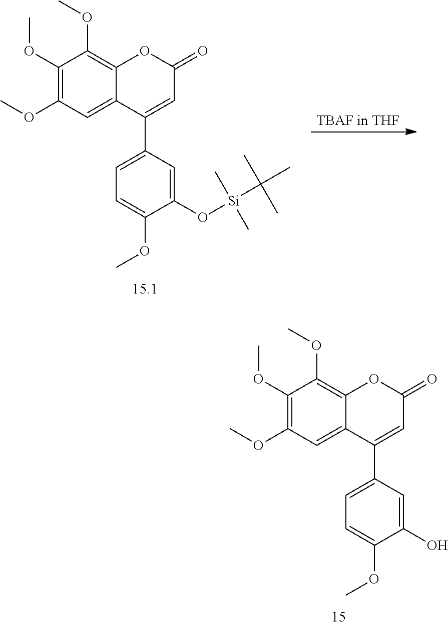

To a stirred solution of 15.1 (30 mg, 0.06 mmol) in THF (2 mL) was added 1M TBAF (0.06 mL, 0.06 mmol) at 0° C. After 2 h the reaction was quenched by the addition of sat. aq. NaCl (1×20 mL) and the product was extracted with diethyl ether (3×20 mL). The ether extracts were combined, dried over MgSO₄ and filtered. The organic fractions were applied directly to a flash column, without prior concentration of the solution in vacuo. The product was purified by flash column chromatography (stationary phase; silica gel 230-400 mesh, mobile phase; 2:1, hexane/ethyl acetate). All homogenous fractions were collected and the solvent was evaporated to afford 15 as a yellow solid (20 mg, 93%).

¹H NMR (CDCl₃, 400 MHz) δ_{H} ppm: 3.78 (3H, s, OMe), 4.01 (3H, s, OMe), 4.03 (3H, s, OMe), 4.08 (3H, s, OMe), 5.81 (1H, s, br, OH), 6.29 (1H, s, C═CH), 6.79 (1H, s, ArH {A-ring}), 6.99 (2H, m, 2×ArH {C-ring}), 7.07 (1H, s, ArH {C-ring})

¹³C NMR (CDCl₃, 400 MHz) δ_{c} ppm: 55.62 (OMe), 55.88 (OMe), 61.07 (OMe), 61.47 (OMe), 102.89 (ArCH), 110.35 (ArCH), 113.12 (C═CH), 114.15 (ArCH), 120.05 (ArCH), 128.25 (ArC), 140.90 (ArC), 142.97 (ArC), 145.41 (2×ArC), 147.25 (ArC), 149.10 (ArC), 154.82 (ArC), 160.32 (C═O)

νₘₐₓ (KBr)/cm⁻¹ 3373.5, 2924.3, 1721.5, 1389.1

Melting Point: 152-157° C.

HRMS: calculated 381.0950, found 381.0944, elemental composition (C₁₉H₁₈O₇Na).

### Alternative Preparation of 15 from 15.1

To a stirred solution of 15.1 (30 mg, 0.06 mmol) in DMF was added sodium azide (46 mg, 0.70 mmol, 10 eq.). The reaction was allowed to proceed at 60° C. for 24 h before being quenched with water (5 mL). Following extraction with ether and purification by column chromatography, 15 was isolated as a yellow crystalline material, (20 mg, 93%), with identical physical properties to that obtained from the TBAF mediated deprotection.

¹H NMR (CDCl₃, 400 MHz) δ_{H} ppm: 3.78 (3H, s, OMe), 4.01 (3H, s, OMe), 4.03 (3H, s, OMe), 4.08 (3H, s, OMe), 5.81 (1H, s, br, OH), 6.29 (1H, s, C═CH), 6.79 (1H, s, ArH {A-ring}), 6.99 (2H, m, 2×ArH {C-ring}), 7.07 (1H, s, ArH {C-ring})

¹³C NMR (CDCl₃, 400 MHz) δ_{c} ppm: 55.62 (OMe), 55.88 (OMe), 61.07 (OMe), 61.47 (OMe), 102.89 (ArCH), 110.35 (ArCH), 113.12 (C═CH), 114.15 (ArCH), 120.05 (ArCH), 128.25 (ArC), 140.90 (ArC), 142.97 (ArC), 145.41 (2×ArC), 147.25 (ArC), 149.10 (ArC), 154.82 (ArC), 160.32 (C═O)

νₘₐₓ (KBr)/cm⁻¹ 3373.5, 2924.3, 1721.5, 1389.1

Melting Point: 152-157° C.

HRMS: calculated 381.0950, found 381.0944, elemental composition (C₁₉H₁₈O₇Na).

FIG. 1a-e: The progress of both synthetic steps was monitored by NMR using sodium azide (10 eq) and dDMF as solvent FIG. 1a-e. Immediate substitution of bromide takes place to give a mixture of 14.1 and azide intermediate. Gradual consumption of the starting material is accompanied by sequential formation of the coumarin backbone from the azide intermediate. Complete conversion to 15.1 is seen after 35 min. The NMR tube was then heated to 60° C. for 4 h resulting in complete deprotection to give 15.

### Synthesis of (Z)-5-(3-amino-4-methoxyphenyl)-7,8,9-trimethoxybenzo[b]oxepin-3(2H)-one 16

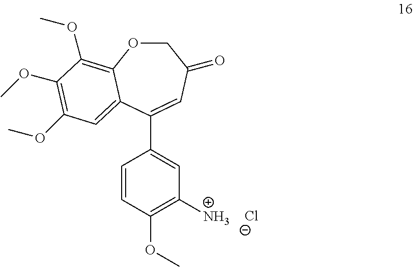 [SMILES: COc1ccc(C2=CC(=O)COc3c2cc(OC)c(OC)c3OC)cc1[NH3+] ; [Cl-]]

### Synthesis of the triflate of the tert-butyl diphenyl silyl protected 3,4-dihydro-3-hydroxy-7, 8,9-trimethoxybenzo[b]oxepin-5(2H)-one 16.1

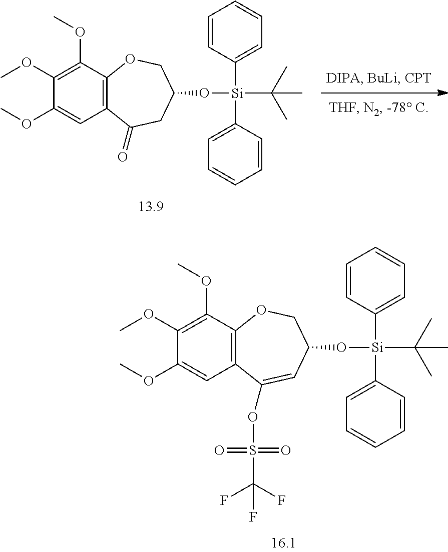

To a dry three-necked round bottom flask containing N,N-diisopropylamine (0.13 mL, 0.91 mmol) in anhydrous THF (3 mL) was added 2.5M nBuLi (0.36 mL, 0.91 mmol) under dry reaction conditions at −78° C. After twenty minutes a solution of the ketone 13.9 (460 mg, 0.91 mmol) in dry THF (5 mL) was transferred to the three-necked flask, drop-wise via a syringe. The resultant suspension was allowed to stir at −78° C. for 2 hr and a solution of 2-[N,N-bis(trifluoromethylsulfonyl)amino]-5-chloropyridine (130 mg, 0.33 mmol) in dry THF (3 mL) was added. The reaction was allowed to stir for an additional 3 hr at this temperature. The reaction was quenched by the addition of water (1×50 mL) and extracted with diethyl ether (3×50 mL). The combined organic fractions were dried over MgSO₄, filtered and dried under vacuum. The residue was purified by flash column chromatography (stationary phase; silica gel 230-400 mesh, mobile phase; 8:1, hexane/ethyl acetate) to yield the triflate

16.1 as a colourless oil (520 mg, 90%).

¹H NMR (CDCl₃, 400 MHz) δ_{H} ppm: 1.12 (9H, s, C(CH₃)₃), 3.86 (3H, s, OMe), 3.91 (3H, s, OMe), 3.96 (3H, s, OMe), 4.19 (1H, d, J=4 Hz CH₂), 4.23 (1H, d, J=4 Hz, CH₂), 4.69 (1H, q, CHOSi), 6.00 (1H, d, J=4 Hz, C═CH), 6.84 (1H, s, ArH), 7.36-7.55 (10H, m, 10×ArH)

¹³C NMR (CDCl₃, 400 MHz) δ_{c} ppm: 19.2 (C(CH₃)₃), 26.6 (C(CH₃)₃), 26.8 (2×C(CH₃)₃), 56.1 (OMe), 61.3 (OMe), 61.8 (OMe), 67.7 (CHOSi), 74.6 (CH₂), 104.3 (C═CH), 117.5 (ArC), 124.9 (ArCH (A-ring)), 127.7 (ArCH), 128.0 (2×ArCH), 129.7 (ArCH), 130.2 (ArCH), 132.8 (CF₃), 132.9 (ArC), 134.8 (2×ArCH), 135.2 (ArC), 135.7 (2×ArCH), 135.8 (2×ArCH), 143.5 (ArC), 144.5 (ArC), 145.0 (ArC), 147.0 (ArC), 148.8 (ArC),

¹⁹F NMR (CDCl₃, 400 MHz) δ_{F} ppm: −74.49

νₘₐₓ (DCM)/cm⁻¹ 3467.3, 2932.3, 1595.0, 1419.8, 1211.8, 1113.4

### Suzuki Coupling of the tetra-hydro benzo-oxepin-5-one triflate 16.1 and the Boronic Ester 13.15

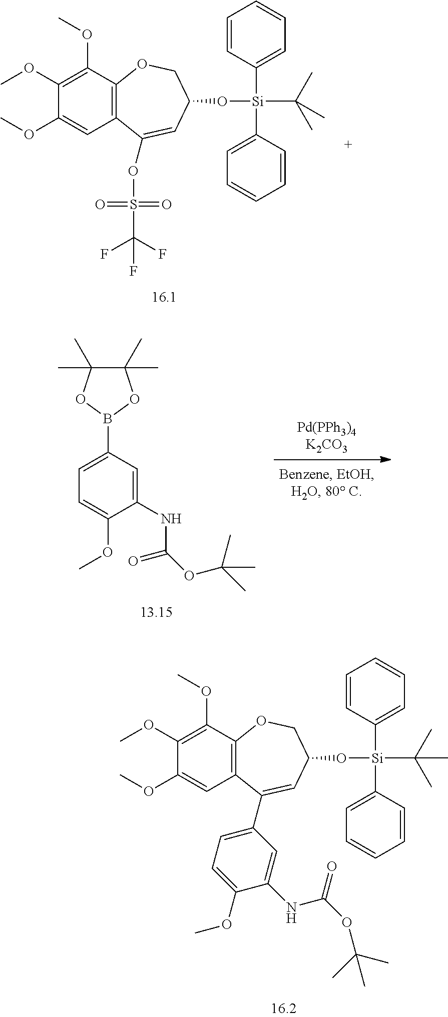

To a flask containing triflate 16.1 (100 mg, 0.16 mmol) was added boronic acid ester 13.15 (66 mg, 0.19 mmol), K₂CO₃ (60 mg, 0.42 mmol), and tetrakis-(triphenylphosphine)-palladium (0) (1 mg, 0.008 mmol). The mixture was dissolved in a mixture of benzene (3 mL), ethanol (1 mL) and water (1 mL) and heated to 70° C. for 30 min. The reaction was quenched by the addition of water (1×5 mL) and the product was extracted with diethyl ether (3×15 mL). The organic fractions were combined, dried over MgSO₄, filtered and concentrated in vacuo. The resulting residue was purified by flash column chromatography (stationary phase; silica gel 230-400 mesh, mobile phase; 8:1, hexane/ethyl acetate). All homogenous fractions were collected and the solvent was evaporated to afford 16.2 as a yellow oil (114 mg, 100%).

¹H NMR (CDCl₃, 400 MHz) δ_{H} ppm: 1.11 (9H, s, C(CH₃)₃ (tBuDiSi)), 1.54 (9H, s, C(CH₃)₃ (tBOC)), 3.62 (3H, s, OMe {C-ring}), 3.87 (3H, s, OMe), 3.91 (6H, s, 2×OMe), 4.33 (2H, d, CH₂), 4.52 (1H, m, CHOSi), 6.25 (1H, d, J=5.0 Hz, C═CH), 6.77 (1H, d, J=2 Hz, ArH), 6.78 (1H, s, ArH), 7.07 (1H, s, ArH), 7.35-7.46 (6H, m, 6×ArH), 7.64-7.75 (4H, m, 4×ArH), 8.06 (1H, br, NH).

¹³C NMR (CDCl₃, 400 MHz) δ_{c} ppm: 19.4 (C(CH₃)₃), 27.2 (3×C(CH₃)₃ (tBuDiSi)), 28.2 (3×C(CH₃)₃ (tBOC)), 55.8 (OMe), 56.2 (OMe), 61.1 (OMe), 61.8 (OMe), 70.1 (CHOSi), 81.1 (CH₂), 108.9 (C(CH₃)₃ (BOC)), 109.4 (ArCH), 118.7 (ArCH), 122.8 (ArCH), 127.7 (4×ArCH), 129.7 (2×ArCH), 129.8 (ArCH), 132.0 (ArCH), 133.5 (ArC), 133.7 (ArC), 134.8 (ArC), 135.0 (ArC), 135.7 (2×ArCH), 135.9 (2×ArCH), 137.6 (2×ArC), 142.2 (ArC), 145.7 (ArC), 145.9 (ArC), 147.1 (ArC), 148.5 (ArC), 152.6 (C═O).

### Synthesis of Intermediate tert-butyl N-[5-(3-hydroxy-7,8,9-trimethoxy-2,3-dihydro-1-benzoxepin-5-yl)-2-methoxyphenyl]carbamate 16.3

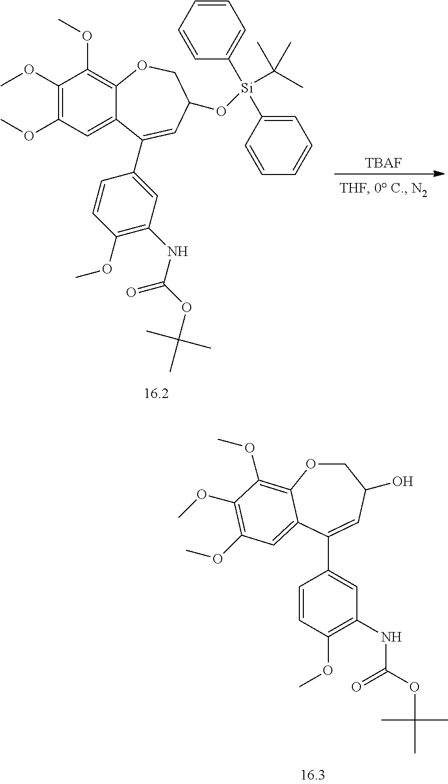

Silyl ether tert-butyl N-(5-{3-[(tert-butyldiphenylsilyl)oxy]-7,8,9-trimethoxy-2,3-dihydro-1-benzoxepin-5-yl}-2-methoxyphenyl)carbamate 16.2 (0.63 g, 0.885 mmoles) was dissolved in anhydrous THF (10 mL) under an atmosphere of nitrogen. To this tetrabutylammonium fluoride (1.1 mL, 1 M, 1.06 mmoles) was added dropwise and the reaction cooled to 0° C. After 2 h the reaction was loaded directly onto silica and purified by column chromatography (1:1, hexane:ethyl acetate) to afford alcohol 16.3 (0.41 g, 0.87 mmoles, 98%) as a clear oil.

¹H NMR (400 MHz, CHLOROFORM-d) δH: 1.53 (9H, s, (CH₃)₃), 3.59 (3H, s, OCH₃), 3.92 (3H, s, OCH₃), 3.95 (3H, s, OCH₃), 3.98 (3H, s, OCH₃), 4.06-4.56 (2H, ddd, CH₂ J=196, 12.0, 2.5 Hz), 4.43-4.50 (1H, m, CHOH), 6.17 (1H, d, CH═C, J=4.5 Hz), 6.30 (1H, s, ArH), 6.83 (2H, s, ArH), 7.13 (1H, s, ArH), 8.10 (1H, br. s, NH).

¹³C NMR (101 MHz, CHLOROFORM-d) δC: 27.9 (C(CH₃)₃), 55.3 (OCH₃), 55.8 (OCH₃), 60.8 (OCH₃), 61.4 (OCH₃), 70.0 (CHOH), 78.4 (CH₂), 79.9 (C(CH₃)₃), 108.8 (ArCH), 109.6 (ArCH), 118.3 (CH═C), 122.7 (ArCH), 125.2 (ArC), 127.3 (ArC), 130.7 (ArCH), 135.9 (ArC), 138.6 (ArC), 142.1 (ArC), 144.6 (ArC), 146.4 (C═C), 147.7 (ArC), 147.7 (ArC), 152.2 (C═O)

MS: calculated 473.2050, found 496.1962 (M+Na⁺).

### Synthesis of tert-butyl N-[2-methoxy-5-(7,8,9-trimethoxy-3-oxo-2H-1-benzoxepin-5-yl)phenyl]carbamate 16.4

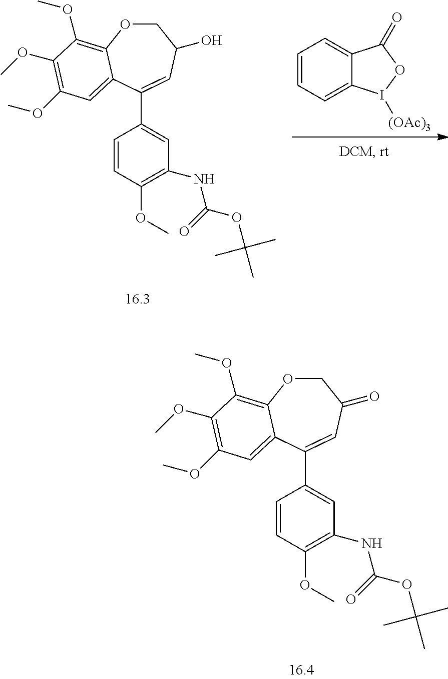

Alcohol 16.3 (0.41 g, 0.87 mmoles) was dissolved in DCM (20 mL) and Dess-Martin periodinane (1.1 g, 2.6 mmoles) was added. The reaction was stirred at rt for 5 min. The reaction was then quenched with aq. sodium hydrogencarbonate solution (50 mL, 5%) and extracted with diethyl ether (4×50 mL), dried with MgSO₄, filtered and condensed in vacuo. Ketone product 16.4 (0.35 g, 0.742 mmoles, 85%) was eventually obtained following column chromatography (3:1, hexane:ethyl actetate) as a sticky yellow oil.

¹H NMR (400 MHz, CHLOROFORM-d) δH:1.53 (9H, s, C(CH₃)₃), 3.65 (3H, s, OCH₃), 3.95 (3H, s, OCH₃), 4.00 (3H, s, OCH₃), 4.01 (3H, s, OCH₃), 4.64 (2H, s, CH₂), 6.38 (1H, s, CH═C), 6.50 (1H, s, ArH), 6.88 (1H, d, ArH, J=8.5 Hz), 6.98 (1H, d, ArH, J=7.5 Hz), 7.12 (1H, s, ArH), 8.16 (1H, br. s, NH)

¹³C NMR (101 MHz, CHLOROFORM-d) δC, 27.9 (C(CH₃)₃), 55.4 (OCH₃), 55.8 (OCH₃), 60.9 (OCH₃), 61.5 (OCH₃), 80.2 (C(CH₃)₃), 80.5 (CH₂), 109.0 (ArCH), 110.3 (CH═C), 118.5 (ArCH), 123.2 (ArCH), 125.7 (ArC), 127.6 (ArC), 127.9 (ArCH), 133.9 (ArC), 144.1 (ArC), 144.6 (ArC), 147.1 (ArC), 147.9 (ArC), 148.6 (ArC), 151.7 (C═CH), 152.1 (BOC—C═O), 199.9 (CH₂C═O)

MS: calculated 471.1893, found 472.1968 (M+H⁺).

### Synthesis of 2-methoxy-5-(7,8,9-trimethoxy-3-oxo-2H-1-benzoxepin-5-yl)anilinium chloride 16

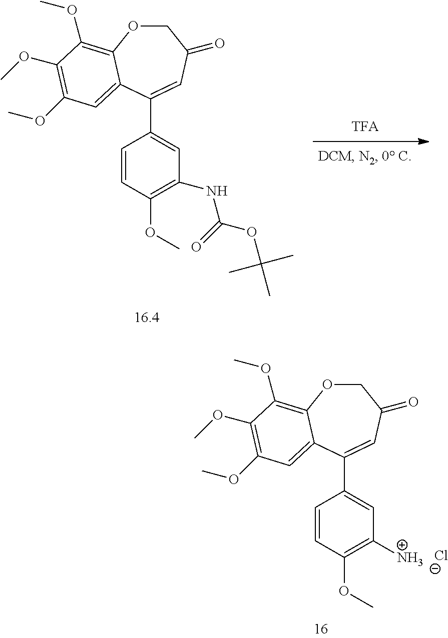

To carbamate 16.4 (15 mg, 0.032 mmoles) in a round bottomed flask flushed with nitrogen, was added trifluoroacetic acid in DCM (1:1, 1 mL) and the reaction cooled to 0° C. After 5 min the reaction was dried, blown with nitrogen gas. The residue was then redissolved in diethyl ether (10 mL) and washed with sodium hydrogencarbonate (1 mL). The organic layer was concentrated in vacuo and HCl gas blown through, to afford anilinium salt 16 (9.1 mg, 0.022 mmoles, 70%) as a yellow solid.

¹H NMR (400 MHz, CHLOROFORM-d) δH: 3.09 (3H, s, OCH₃), 3.65 (3H, s, OCH₃), 3.93 (3H, s, OCH₃), 3.99 (3H, s, OCH₃), 4.01 (3H, s, OCH₃), 4.64 (2H, s, CH₂), 6.40 (1H, s, CH═C), 6.46 (1H, s, ArH), 6.72-6.84 (3H, m, 3×ArH)

¹³C NMR (101 MHz, CHLOROFORM-d) δC: 55.1 (OCH₃), 55.8 (OCH₃), 60.9 (OCH₃), 61.5 (OCH₃), 80.7 (CH₂), 109.3 (ArCH), 110.1 (ArCH), 115.1 (CH═C), 119.5 (ArCH), 125.1 (ArC), 125.9 (ArC), 127.4 (ArCH), 133.8 (ArC), 143.9 (ArC), 144.7 (ArC), 146.9 (ArC), 147.8 (ArC), 148.6 (ArC), 152.0 (C═CH), 200.3 (C═O)

MS: calculated 371.1369, found 372.9862 (M+H⁺).

νₘₐₓ (DCM)/cm⁻¹: 3374.4, 2932.5, 2852.9, 1657.4

### Synthesis of tert-butyl N-[5-(2-bromo-7,8,9-trimethoxy-3-oxo-2H-1-benzoxepin-5-yl)-2-methoxyphenyl]carbamate 17.1.

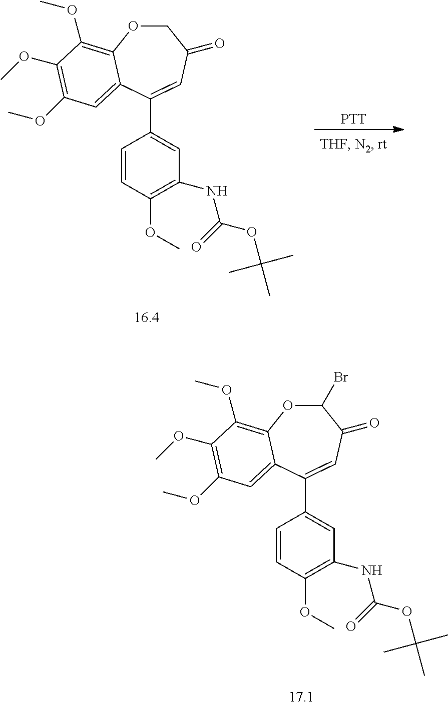

To a solution of tert-butyl N-[2-methoxy-5-(7,8,9-trimethoxy-3-oxo-2H-1-benzoxepin-5-yl)phenyl]carbamate 16.4 (0.11 g, 0.233 mmoles) in dry THF (3 mL), at room temperature and under an atmosphere of nitrogen, was added dropwise phenyltrimethylammonium tribromide (0.11 g, 0.303 mmoles) in dry THF (3 mL) and the reaction progress monitored by TLC. After approximately 1 h the reaction was quenched with cold water (50 mL) and extracted with diethyl ether (3×50 mL) before drying with MgSO₄ and concentration under reduced pressure. The reaction mixture was then purified by column chromatography (3:1 hexane:ethyl acetate) to afford tert-butyl N-[5-(2-bromo-7,8,9-trimethoxy-3-oxo-2H-1-benzoxepin-5-yl)-2-methoxyphenyl]carbamate 17.1 (87 mg, 0.16 mmol, 68%) as a viscous yellow oil.

¹H NMR (400 MHz, CHLOROFORM-d) δH: 3.66 (3H, s, OCH₃), 3.96 (3H, s, OCH₃), 4.02 (3H, s, OCH₃), 4.02 (3H, s, OCH₃), 6.44 (1H, s, ArH), 6.49 (1H, d, CH═C, J=1.00 Hz), 6.72 (1H, d, CHBr J=1.00 Hz), 6.89 (1H, d, ArH, J=8.53 Hz), 6.99 (1H, dd, ArH J=8.53, 2.01 Hz), 7.14 (1H, s, ArH), 8.17 (1H, br. s, NH)

¹³C NMR (101 MHz, CHLOROFORM-d) δC: 27.9 (C(CH₃)₃), 55.4 (OCH₃), 55.7 (OCH₃), 60.8 (OCH₃), 61.3 (OCH₃), 80.3 (C(CH₃)₃), 85.8 (CBr), 109.0 (ArCH), 110.2 (ArCH), 118.5 (CH═C), 123.3 (ArCH), 125.6 (ArC), 125.7 (ArCH), 127.6 (ArC), 133.7 (ArC), 141.8 (ArC), 144.6 (ArC), 145.7 (ArC), 148.0 (ArC), 149.4 (ArC), 152.1 (C═CH), 152.7 (NC═O), 190.2 (C═O)

νₘₐₓ (DCM)/cm⁻¹: 3430.5, 2977.8, 2938.9, 2843.8, 1724.9, 1650.7, 1528.5

### Ring Contraction of 7-Membered tert-butyl N-[5-(2-bromo-7,8,9-trimethoxy-3-oxo-2H-1-benzoxepin-5-yl)-2-methoxyphenyl]carbamate to Give 6-Membered tert-butyl 2-methoxy-5-(6,7,8-trimethoxy-2-oxo-2H-chromen-4-yl)phenylcarbamate 17.2

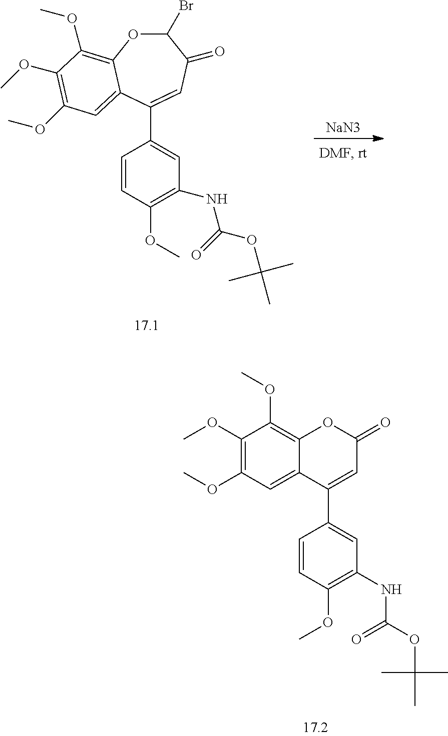

Bromide 17.1 (18 mg, 0.033 mmoles) was dissolved in DMF (5 mL) at room temperature. Sodium azide (10 mg, 0.165 mmoles) was then added and the reaction allowed stir while being monitored by TLC. Upon completion the reaction was quenched with water (30 mL) and extracted with ditheyl ether (3×30 mL) to give chromenone product 17.2 (9 mg, 0.022 mmoles, 60%) as a yellow oil.

¹H NMR (400 MHz, CHLOROFORM-d) δH: 1.17 (9H, s, (CH₃)₃), 1.45 (6H, s, Si(CH₃)₂), 3.75 (3H, s, OCH₃), 3.91 (3H, s, OCH₃), 3.95 (3H, s, OCH₃), 3.98 (3H, s, OCH₃), 6.24 (1H, s, C═C), 6.90 (1H, s, ArH), 6.93-6.97 (1H, m, ArH), 7.04 (1H, dd, ArH, J=8.53, 2.01 Hz), 7.15 (1H, s, ArH), 8.24 (1H, br. s, NH)

¹³C NMR (101 MHz, CHLOROFORM-d) δC, 24.3 (Si(CH₃)₂), 27.7 (C(CH₃)₃), 55.4 (OCH₃), 55.6 (OCH₃), 60.9 (OCH₃), 61.3 (OCH₃), 80.2 (C(CH₃)₂), 103.1 (ArCH), 109.8 (ArH), 112.6 (CH═C), 113.9 (ArC), 118.0 (ArCH), 122.1 (ArCH), 127.4 (ArC), 127.7 (2×ArC), 140.7 (ArC), 142.7 (ArC), 145.2 (ArC), 148.3 (ArC), 149.1 (ArC), 152.2 (C═CH), 155.0 (NC═O), 160.4 (OC═O)

MS: calculated 457.1737, found 480.1620 (M+Na⁺).

### Synthesis of 4-(3-amino-4-methoxyphenyl)-6,7,8-trimethoxy-2H-chromen-2-one 17

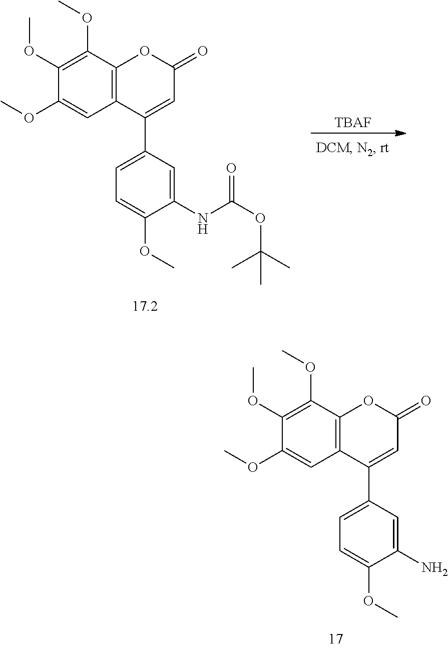

Boc-protected aniline 17.2 (0.097 g, 0.00021 moles) was then reacted with a dry DCM:trifluoroacetic acid (1:1, 1 mL) mixture in a roundbottom flask flushed with nitrogen. After 75 minutes stirring DCM:trifluoroacetic acid mixture was removed in vacuo. The remainder was then basified with sodium hydrogencarbonate solution (50 ml, 5%) and extracted with diethyl ether. A salt of the compound was then made from conc H₂SO₄\HCl and impurities removed with diethyl ether. Aniline compound 17 (0.049 g, 0.000138 moles) was hence obtained as a brown solid.

¹H NMR (CDCl₃, 400 MHz) δH: 3.05 (2H, br s, NH₂), 3.75 (3H, s, OCH₃), 3.94 (3H, s, OCH₃), 4.0 (3H, s, OCH₃), 4.04 (3H, s, OCH₃), 6.25 (1H, s, C═CH), 6.79 (1H, s, ArH), 6.81 (2H, s, 2×ArH), 6.9 (1H, d, ArH, J=8.11 Hz)

¹³C NMR (CDCl₃, 400 MHz) δC: 55.63 (OCH₃), 56.37 (OCH₃), 61.52 (OCH₃), 61.92 (OCH₃), 103.6 (ArCH), 110.23 (C═CH), 113.24 (ArCH), 114.72 (2×ArC) 118.75 (ArCH), 125.54 (ArCH), 128.2 (ArC), 141.32 (ArC), 143.4 (ArC), 145.79 (ArC), 149.49 (2×ArC), 155.93 (C═CH), 160.94 (C═O)

MS: 358.2184 (M+H⁺), 380.1364 (M+Na⁺)

### Synthesis of tert-butyl N-(1-{[2-methoxy-5-(7,8,9-trimethoxy-3-oxo-2H-1-benzoxepin-5-yl)phenyl]carbarnoyl}-3-methylbutyl)carbamate 18

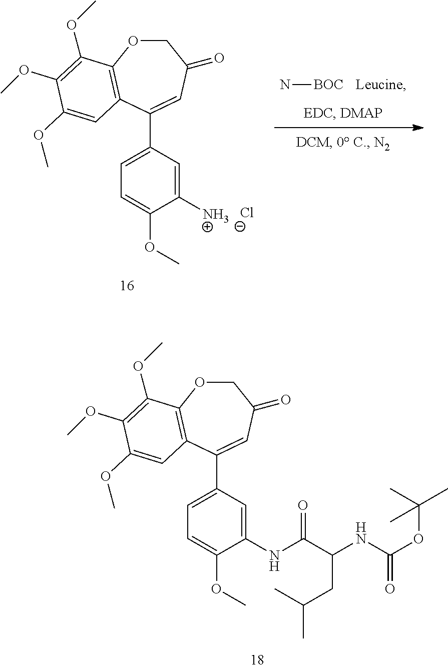 [SMILES: COc1ccc(C2=CC(=O)COc3c2cc(OC)c(OC)c3OC)cc1[NH3+] ; [Cl-] ; CC(C)(C)OC(N)=O ; COc1ccc(C2=CC(=O)COc3c2cc(OC)c(OC)c3OC)cc1NC(=O)C(CC(C)C)NC(=O)OC(C)(C)C]

Amine salt 16 (70 mg, 0.172 mmoles) was dissolved in anhydrous DCM (3 mL) with anhydrous DMF (0.5 mL) under an atmosphere of nitrogen at 0° C. To this was added sequentially in dry DCM; N—BOC Leucine (0.2 g, 0.86 mmoles), N-(3-dimethylaminopropyl)-N′-ethylcarbodiimide (0.11 g, 0.86 mmoles) and dimethylaminopyridine (16 mg, 0.086 mmoles) and the reaction monitored via TLC. The reaction was then quenched with aq. HCl (20 mL, 1 M) and extracted with diethyl ether (3×30 mL). The organic layer was then dried with MgSO₄, filtered and concentrated in vacuo before the residue was then purified by column chromatography (3:1, hexane:ethyl acetate) to give carbamate product 18 as a light brown oil (70 mg, 0.12 mmoles, 70%)

¹H NMR (600 MHz, CHLOROFORM-d) δH: 0.99 (6H, t, 2×CH₃, J=6.40 Hz), 1.49 (9H, s, (CH₃)₃), 1.53-1.82 (2H, m, Leu-CH₂), 1.69-1.78 (1H, m, CH(CH₃)₂), 3.64 (3H, s, OCH₃), 3.95 (3H, s, OCH₃), 3.99 (3H, s, OCH₃), 4.00 (3H, s, OCH₃), 4.63 (2H, s, OCH₂C═O), 4.68 (1H, ddd, COCHNH, J=3.40 Hz), 4.97 (1H, br. s, ArNH), 5.29 (1H, d, CHNH, J=8.66 Hz), 6.37 (1H, s, ArH), 6.47 (1H, s, CH═C), 6.91 (1H, d, ArH, J=8.66 Hz), 7.07 (1H, d, ArH, J=7.53 Hz), 8.42 (1H, s, ArH)

¹³C NMR (151 MHz, CHLOROFORM-d) δC: 23.3 (CH(CH₃)₂), 24.5 (CH(CH₃)₂), 28.2 (C(CH₃)₃), 40.9 (Leu-CH₂), 48.5 (CHNH), 55.8 (OCH₃), 56.2 (OCH₃), 61.1 (OCH₃), 61.7 (OCH₃), 79.2 (C(CH₃)₃), 80.8 (OCH₂), 109.5 (ArCH), 110.6 (ArCH), 120.7 (ArCH), 124.9 (ArCH), 125.9 (ArC), 128.4 (CH═C), 134.2 (ArC), 144.5 (ArC), 145.0 (ArC), 147.4 (ArC), 148.9 (ArC), 149.0 (2×ArC), 151.7 (C═CH), 155.7 (BOC—C═O), 170.6 (ArNC═O), 200.1 (CH₂C═O).

MS: calculated 584.2734, found 583.2708 (M−H⁺).

### Synthesis of [(5-{3-[(tert-butyldimethylsilyl)oxy]-4-methoxyphenyl}-7,8,9-trimethoxy-3-oxo-2,3-dihydro-1-benzoxepin-2-yl)sulfanyl]formonitrile 19.1

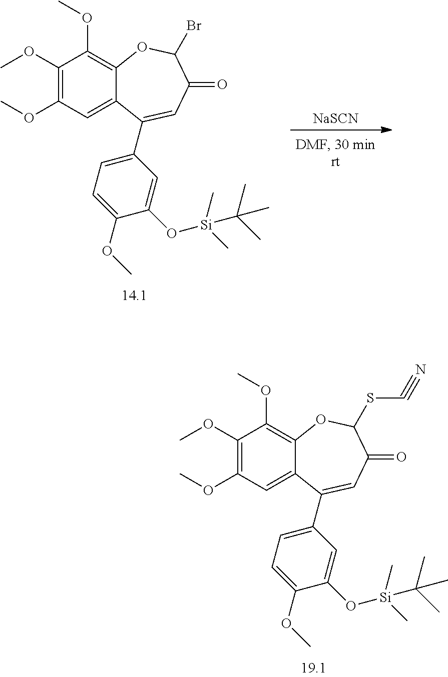 [SMILES: COc1ccc(C2=CC(=O)C(Br)Oc3c2cc(OC)c(OC)c3OC)cc1O[Si](C)(C)C(C)(C)C ; "NaSCN" ; COc1ccc(C2=CC(=O)C(SC#N)Oc3c2cc(OC)c(OC)c3OC)cc1O[Si](C)(C)C(C)(C)C]

To 2-bromo-5-{3-[tert-butyldimethylsilyl)oxy]-4-methoxyphenyl}-7,8,9-trimethoxy-2,3-dihydro-1-benzoxepin-3-one 14.1 (90 mg, 0.16 mmol) in DMF (1 mL) was added sodium thiocyanate (19 mg, 0.24 mmol) with stirring for 30 min. The reaction mixture was then washed with 5% lithium chloride solution (50 mL) and extracted with diethyl ether (3×50 mL). The organic layers were then dried with magnesium sulphate and condensed under reduced pressure before the crude material was purified by column chromatography (3:1, hexane:ethyl acetate). The resultant target molecule [(5-{3-[tert-butyldimethylsilyl)oxy]-4-methoxyphenyl}-7,8,9-trimethoxy-3-oxo-2,3-dihydro-1-benzoxepin-2-yl)sulfanyl]formonitrile 19.1 (50 mg, 0.092 mmol, 58%) was then obtained as a viscous yellow oil.

1H NMR (CDCl3, 400 MHz) δH: 0.19 (6H, s, 2×SiCH₃), 1.02 (9H, s, C(CH₃)₃), 3.66 (3H, s, OCH₃), 3.90 (3H, s, OCH₃), 4.03 (3H, s, OCH₃), 4.10 (3H, s, OCH₃), 6.15 (1H, s, CHSCN), 6.38 (1H, s, C═CH), 6.48 (1H, s, ArCH), 6.86 (1H, s, ArCH), 6.9 (1H, d, ArCH, J=8.35 Hz), 6.95 (1H, d, ArCH, J=8.35 Hz)

13C NMR (CDCl3, 400 MHz) δC: −4.6 (2×SiCH₃), 18.4 (C(CH₃)₃), 25.6 (3×C(CH₃)₃), 55.4 (OCH₃), 56.2 (OCH₃), 61.4 (OCH₃), 62.0 (OCH₃), 93.0 (CHS), 109.2 (SCN), 110.5 (ArCH), 111.4 (ArCH), 121.8 (C═CH), 123.3 (ArCH), 125.5 (ArCH), 133.4 (ArC), 143.1 (ArC), 144.8 (ArC), 145.3 (C═C), 146.1 (ArC), 150.4 (ArC), 152.5 (ArC), 154.1 (ArC), 191.4 (C═O)

νₘₐₓ (DCM)/cm⁻¹: 2930.65, 2856.79, 1652.99, 1509.55

HRMS m/z 543.7654

### Synthesis of 5-{3-[(tert-butyldimethylsilyl)oxy]-4-methoxyphenyl}-2-(ethylsulfanyl)-7,8,9-trimethoxy-2,3-dihydro-1-benzoxepin-3-one 20.1

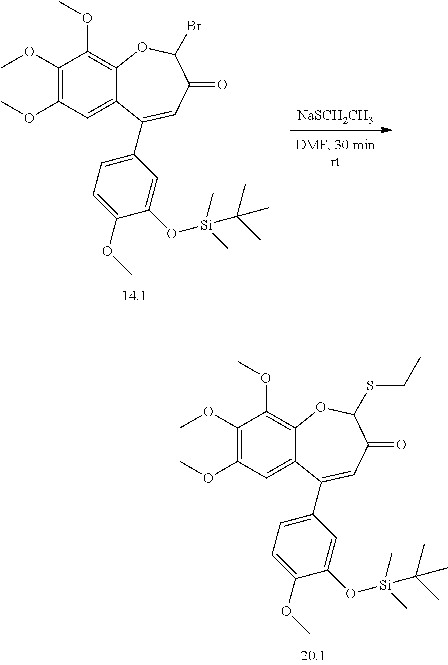 [SMILES: COc1ccc(C2=CC(=O)C(Br)Oc3c2cc(OC)c(OC)c3OC)cc1O[Si](C)(C)C(C)(C)C ; "NaSCH2CH3" ; CCSC1Oc2c(cc(OC)c(OC)c2OC)C(c2ccc(OC)c(O[Si](C)(C)C(C)(C)C)c2)=CC1=O]

To 2-bromo-5-{3-[(tert-butyldimethylsilyl)oxy]-4-methoxyphenyl}-7,8,9-trimethoxy-2,3-dihydro-1-benzoxepin-3-one 14.1 (80 mg, 0.141 mmol) in DMF (1 mL) was added sodium ethanethiolate (18 mg, 0.212 mmol) with stirring for 30 min. The reaction mixture was then washed with 5% lithium chloride solution (50 mL) and extracted with diethyl ether (3×50 mL). The organic layers were then dried with magnesium sulphate and condensed under reduced pressure before the crude material was purified by column chromatography (3:1, hexane:ethyl acetate). The resultant target 5-{3-[(tert-butyldimethylsilyl)oxy]-4-methoxyphenyl}-2-(ethylsulfanyl)-7,8,9-trimethoxy-2,3-dihydro-1-benzoxepin-3-one 20.1 (45 mg, 0.083 mmol, 59%) was then obtained as a viscous yellow oil.

¹H NMR (CDCl3, 400 MHz) δH: 0.19 (61-1, s, 2×SiCH₃), 1.02 (9H, s, C(CH₃)₃), 1.53 (3H, t, SCH₂CH₃), 3.40 (2H, m, SCH₂), 3.66 (3H, s, OCH₃), 3.89 (3H, s, OCH₃), 4.01 (3H, s, OCH₃), 4.07 (3H, s, OCH₃), 5.20 (1H, s, CHS), 6.36 (1H, s, C═CH), 6.52 (1H, s, ArCH), 6.87 (1H, s, ArCH, J=2 Hz), 6.90 (1H, d, ArCH), J=8.5 Hz), 6.97 (1H, dd, ArCH, J=8.5 Hz, 2 Hz)

¹³C NMR (CDCl₃, 400 MHz) δC: −4.7 (2×SiCH₃), 6.1, 18.3, 25.5, 29.5, 46.4, 55.3 (OCH₃), 56.0 (OCH₃), 61.2 (OCH₃), 61.9 (OCH₃), 96.52 (CHS), 110.0, 111.3, 121.5, 123.0, 125.8, 127.4, 128.6, 130.7, 132.8, 143.6, 144.7, 144.7, 145.2, 150.2, 151.7, 152.3, 192.0 (C═O)

### Synthesis of 7,8,9-trimethoxy-5-(4-methoxyphenyl)-2,3-dihydro-1-benzoxepin-3-ol 21.1

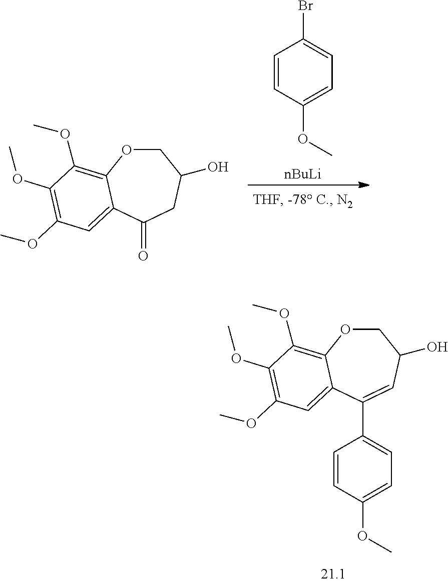

Bromoanisole (2.54 g, 0.0136 moles) was dissolved in dry THF (15 mL) in a 3-necked round bottom flask at −78° C. under an atmosphere of nitrogen. Butyllithium (5.44 mL, 2.5 M, 0.0136 moles) was added dropwise and the reaction allowed to stir at −78° C. for 40 min. Separately, 3-hydroxy-7,8,9-trimethoxy-3,4-dihydro-2H-1-benzoxepin-5-one (0.73 g, 2.72 mmoles) was dissolved in dry THF (10 mL) and then added to the reaction mixture in the 3-necked round bottom flask. After 4 h at −78° C., the reaction was allowed to reach 0° C. and left stirring at this temperature overnight. The reaction was then washed with aq. HCl (50 mL, 1 M) and quickly extracted with diethyl ether (4×50 mL). After drying with MgSO₄, the reaction was concentrated under reduced pressure and purified by column chromatography (2:1, hexane:ethyl acetate) to afford alcohol 21.1 (0.35 g, 0.98 mmoles, 36%) as a yellow solid.

¹H NMR (400 MHz, CHLOROFORM-d) δH: 3.59 (3H, s, OCH₃), 3.86 (3H, s, OCH₃), 3.95 (3H, s, OCH₃), 3.99 (3H, s, OCH₃), 4.11-4.56 (2H, ddd, CH₂, J=180, 11.7, 2.9 Hz), 4.32-4.46 (1H, m, CHOH), 4.49 (1H, br. s, OH), 6.15 (1H, d, CH═C, J=5.1 Hz), 6.26 (1H, s, ArH), 6.90 (2H, d, ArH, J=8.8 Hz), 7.22 (2H, d, ArH, J=8.8 Hz)

¹³C NMR (101 MHz, CHLOROFORM-d) δC: 55.3 (OCH₃), 56.0 (OCH₃), 61.2 (OCH₃), 61.8 (OCH₃), 70.4 (COH), 79.2 (CH₂), 109.6 (ArCH), 113.4 (2×ArCH), 125.7 (ArC), 130.2 (2×ArCH), 130.8 (CH═C), 135.7 (ArC), 138.8 (ArC), 142.6 (ArC), 145.2 (C═C), 148.1 (ArC), 148.2 (ArC), 158.9 (ArC)

MS: calculated 358.1416, found 381.1333 (M+Na⁺).

νₘₐₓ (DCM)/cm⁻¹: 3484.8, 2935.3

### Synthesis of 7,8,9-trimethoxy-5-(4-methoxyphenyl)-2H-1-benzoxepin-3-one 21

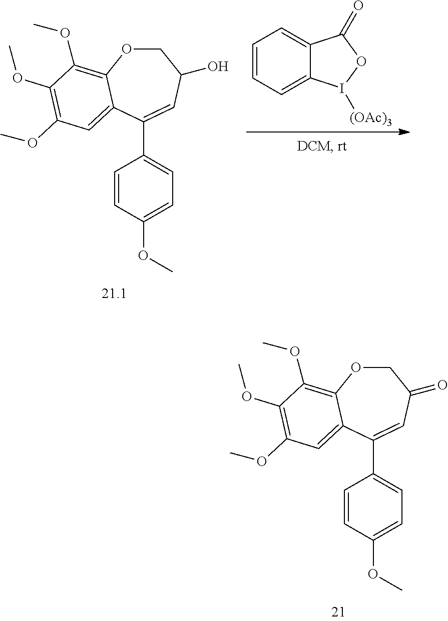

Alcohol 21.1 (0.23 g, 0.642 mmoles) was dissolved in DCM (10 mL) and Dess-Martin periodinane (0.42 g, 0.99 mmoles) was added. The reaction was stirred at rt for 5 min. The reaction was then quenched with aq. sodium bicarbonate solution (50 mL, 5%) and extracted with diethyl ether (4×50 mL). The organic layer was then dried with MgSO₄, filtered and condensed to give product 21 (0.21 g, 0.578 mmoles, 90%) which was obtained in pure form, without column chromatography, as a yellow solid.

¹H NMR (400 MHz, CHLOROFORM-d) δH: 3.64 (3H, s, OCH₃), 3.89 (3H, s, OCH₃), 4.00 (3H, s, OCH₃), 4.02 (3H, s, OCH₃), 4.66 (2H, s, CH₂), 6.33 (1H, s, CH═C), 6.48 (1H, s, ArH), 6.95 (2H, d, 2×ArH, J=8.5 Hz), 7.33 (2H, d, 2×ArH, J=8.5 Hz)

¹³C NMR (101 MHz, CHLOROFORM-d) δC: 54.9 (OCH₃), 55.7 (OCH₃), 60.9 (OCH₃), 61.5 (OCH₃), 80.8 (CH₂), 109.9 (ArCH), 113.3 (2×ArCH), 127.6 (CH═C), 128.8 (ArC), 130.2 (2×ArCH), 133.3 (ArC), 144.1 (ArC), 144.8 (ArC), 147.0 (ArC), 148.7 (C═C), 151.5 (ArC), 160.1 (ArC), 200.2 (C═O)

MS: calculated 356.1260, found 357.1328 (M+H^{+t}); 379.1144 (M+Na⁺).

νₘₐₓ (DCM)/cm⁻¹: 2938.0, 1659.7, 1604.9, 1510.1, 1491.5

### Synthesis of 2-bromo-7,8,9-trimethoxy-5-(4-methoxyphenyl)-2H-1-benzoxepin-3-one 22.1

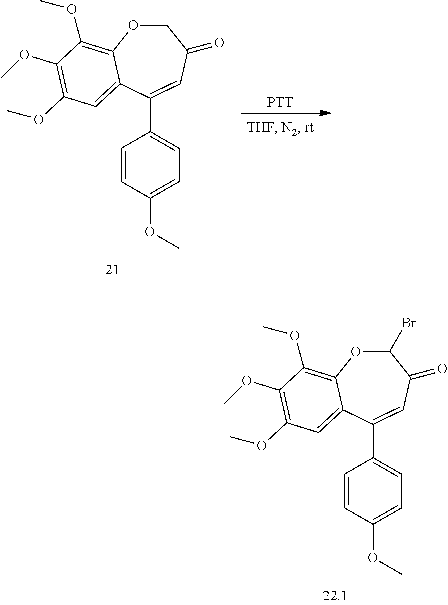 [SMILES: COc1ccc(C2=CC(=O)COc3c2cc(OC)c(OC)c3OC)cc1 ; "PTT" ; COc1ccc(C2=CC(=O)C(Br)Oc3c2cc(OC)c(OC)c3OC)cc1]

To a solution 7,8,9-trimethoxy-5-(4-methoxyphenyl)-2H-1-benzoxepin-3-one 21 (0.18 g, 0.51 mmol) in dry THF (3 mL), at room temperature and under an atmosphere of nitrogen, was added dropwise phenyltrimethylammonium tribromide (0.25 g, 0.66 mmol) in dry THF (3 mL) and the reaction progress monitored by TLC. After approximately 1 h the reaction was quenched with cold water (50 mL) and extracted with diethyl ether (3×50 mL) before drying with MgSO₄ and concentration under reduced pressure. The reaction mixture was then purified by column chromatography (5:1 hexane:ethyl acetate) to afford bromide 2-bromo-7,8,9-trimethoxy-5-(4-methoxyphenyl)-2H-1-benzoxepin-3-one 22.1 (0.15 mg, 0.37 mmol, 72%) as a viscous yellow oil.

¹H NMR (600 MHz, CHLOROFORM-d) δH: 3.65 (3H, s, OCH₃), 3.89 (3H, s, OCH₃), 4.01 (3H, s, OCH₃), 4.03 (3H, s, OCH₃), 6.38 (1H, s, ArH), 6.47 (1H, d, CH═C, J=1.51 Hz), 6.74 (1H, d, CHBr, J=1.51 Hz), 6.97 (2H, d, 2×ArH J=8.66 Hz), 7.33 (2H, d, 2×ArH, J=8.66 Hz)

¹³C NMR (151 MHz, CHLOROFORM-d) δC: 55.4 (OCH₃), 56.1 (OCH₃), 61.2 (OCH₃), 61.7 (OCH₃), 86.4 (CHBr), 110.3 (ArCH), 113.8 (2×ArCH), 125.8 (CH═C), 126.1 (ArC), 130.8 (2×ArCH), 133.6 (ArC), 142.3 (ArC), 145.0 (ArC), 146.3 (ArC), 149.9 (ArC), 152.8 (C═CH), 160.8 (ArC), 190.8 (C═O)

νₘₐₓ (DCM)/cm⁻¹: 2940.8, 2840.9, 2253.2, 1652.8, 1604.9

### Ring Contraction from 7-Membered 2-bromo-7,8,9-trimethoxy-5-(4-methoxyphenyl)-2H-1-benzoxepin-3-one to 6-MEMBERED 6,7,8-trimethoxy-4-(4-methoxyphenyl)-2H-chromen-2-one 22

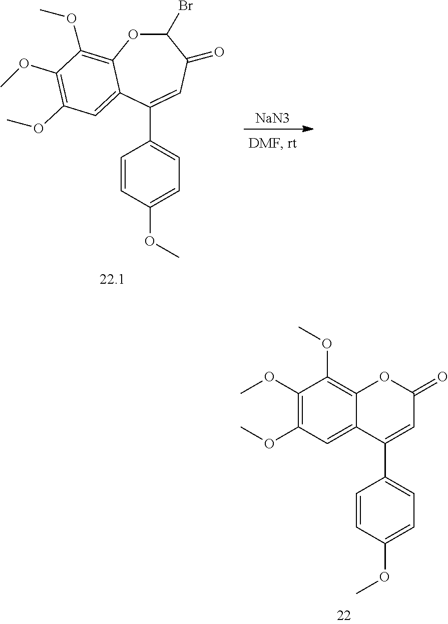 [SMILES: COc1ccc(C2=CC(=O)C(Br)Oc3c2cc(OC)c(OC)c3OC)cc1 ; COc1ccc(-c2cc(=O)oc3c(OC)c(OC)c(OC)cc23)cc1 ; "NaN3"]

Bromide 22.1 (10 mg, 0.023 mmoles) was dissolved in DMF (5 mL) at room temperature. Sodium azide (7 mg, 0.115 mmoles) was then added and the reaction allowed stir while being monitored by TLC. Upon completion the reaction was quenched with water (30 mL) and extracted with ditheyl ether (3×30 mL) to give chromenone product 22 (7 mg, 0.0205 mmoles, 89%) as a yellow powder.

1H NMR (CDCl₃, 400 MHz) δH: 3.77 (3H, s, OCH3), 3.92 (3H, s, OCH3), 4.03 (3H, s, OCH3), 4.07 (3H, s, OCH3), 6.29 (1H, s, C═CH), 6.75 (1H, s, ArH), 7.06 (2H, d, 2×ArH, J=8.6 Hz), 7.42 (2H, d, 2×ArH, J=8.6 Hz)

13C NMR (CDCl₃, 400 MHz) δC: 54.99 (OCH3), 55.82 (OCH3), 61.07 (OCH3), 61.47 (OCH3), 102.77 (ArCH), 113.0 (C═CH), 113.9 (2×ArCH), 114.1 (ArC), 127.32 (ArC), 129.32 (2×ArCH), 140.92 (ArC), 142.98 (ArC), 145.38 (ArC), 149.09 (ArC), 154.89 (C═CH), 160.31 (1×ArC, 1×C═O)

MS: calculated 342.1103, found 365.1034 (M+Na⁺).

### Synthesis of Intermediate 4-{3-[(tert-butyldimethylsilyl)oxy]-4-methoxyphenyl}-6,7,8-trimethoxy-3-(3,4,5-trimethoxyphenyl)chromen-2-one 23.2

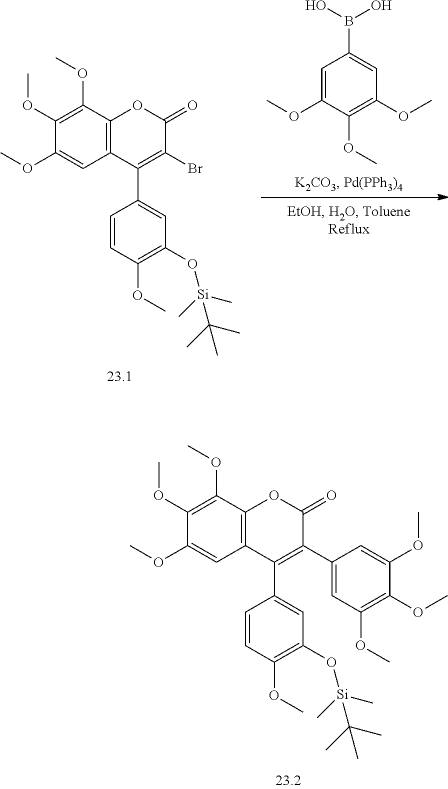

Chromenone 23.1 3-bromo-4-{3-[tert-butyldimethylsilyl)oxy]-4-methoxyphenyl}-6,7,8-trimethoxychromen-2-one (38.4 mg, 0.07 mmoles), boronic acid 3,4,5 trimethoxyphenylboronic acid (22.2 mg, 0.105 mmoles) and potassium carbonate (29 mg, 0.21 mmoles) were dissolved and stirred in a toluene:ethanol:water mixture (3:1:1, 5 mL). To this tetrakis(triphenylphosphine)palladium(0) (4 mg, 3.5 μmoles) was added and the reaction refluxed for 2 h. The reaction was then quenched with brine (50 mL) and extracted with ethyl acetate (3×50 mL) before being dried with MgSO₄ and concentrated in vacuo. After column chromatography (3:1, hexane:ethyl acetate) chromenone product 23.2 was obtained (30.4 mg, 0.0467 mmoles, 68%) as a brown oil.

¹H NMR (600 MHz, CHLOROFORM-d): δH: −0.05 (2H, s, SiCH₃), 0.04 (2H, s, SiCH₃), 0.92 (9H, s, (CH₃)₃), 3.67 (9H, s, 3×OCH₃), 3.80 (3H, s, OCH₃), 3.83 (3H, s, OCH₃), 4.02 (3H, s, OCH₃), 4.09 (3H, s, OCH₃), 6.38 (2H, s, 2×ArH), 6.50 (1H, s, ArH), 6.57 (1H, d, ArH, J=1.9 Hz), 6.80 (1H, dd, ArH, J=8.3, 1.9 Hz), 6.86 (1H, d, ArH, J=8.3 Hz)

¹³C NMR (151 MHz, CHLOROFORM-d) δC=−5.3 (SiCH₃), −5.2 (SiCH₃), 18.1 (C(CH₃)), 25.4 (C(CH₃)₃), 55.3 (OCH₃), 55.7 (2×OCH₃), 56.0 (OCH₃), 60.5 (OCH₃), 61.3 (OCH₃), 61.7 (OCH₃), 103.9 (ArCH), 107.9 (2×ArCH), 111.3 (ArCH), 115.9 (CH═C), 121.8 (ArCH), 122.6 (ArCH), 125.1 (ArC), 127.3 (ArC), 129.4 (ArC), 137.1 (ArC), 140.8 (ArC), 142.1 (ArC), 144.9 (ArC), 145.4 (ArC), 149.4 (C═CH), 151.1 (2×ArC) 152.4 (2×ArC), 160.8 (C═O)

MS: calculated 638.2547, found 639.2620 (M+H⁺).

νₘₐₓ (DCM)/cm⁻¹: 1715.2, 1582.6

### Synthesis of 4-(3-hydroxy-4-methoxyphenyl)-6,7,8-trimethoxy-3-(3,4,5-trimethoxyphenyl)chromen-2-one 23

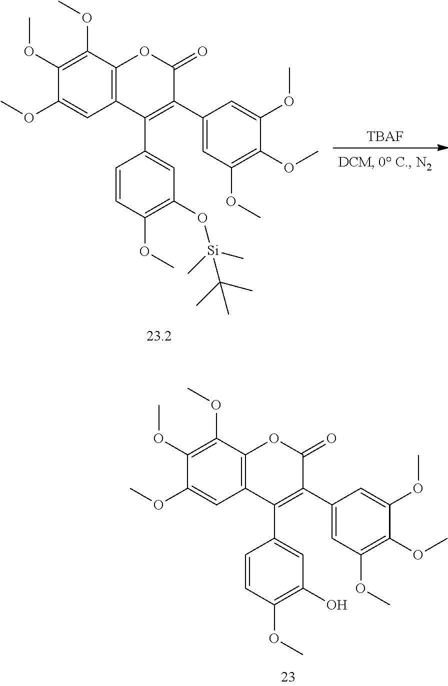

Silyl ether 23.2 4-{3-[tert-butyldimethylsilyl)oxy]-4-methoxyphenyl}-6,7,8-trimethoxy-3-(3,4,5-trimethoxyphenyl)chromen-2-one (70.0 mg, 0.11 mmoles), was dissolved in anhydrous DCM (5 mL) at 0° C., under an atmosphere of nitrogen. To this tetrabutylammonium fluoride (0.12 mL, 0.12 mmol) was added dropwise and the reaction allowed stir for 5 min. The reaction mixture was then transferred directly onto silica and the reaction purified by column chromatography (1:1, hexane:ethyl acetate) to afford phenol 23 (47 mg, 0.088 mmoles, 80%) as an orange solid.

¹H NMR (600 MHz, CHLOROFORM-d) δH: 3.69 (6H, s, 2×OCH₃), 3.72 (3H, s, OCH₃), 3.82 (3H, s, OCH₃), 3.90-3.92 (3H, s, OCH₃), 4.03 (3H, s, OCH₃), 4.11 (3H, s, OCH₃), 5.74 (1H, s, OH), 6.39 (2H, s, 2×ArH), 6.51 (1H, s, ArH), 6.58 (1H, dd, ArH J=8.3, 2.3 Hz), 6.78 (1H, s, ArH), 6.79 (1H, d, ArH J=5.3 Hz)

¹³C NMR (151 MHz, CHLOROFORM-d) δC: 55.7 (OCH₃), 55.8 (2×OCH₃), 56.2 (OCH₃), 60.6 (OCH₃), 61.3 (OCH₃), 61.8 (OCH₃), 104.0 (ArCH), 108.0 (2×ArCH), 110.3 (ArCH), 115.3 (ArCH), 115.9 (C═C), 121.0 (ArCH), 125.3 (ArC), 127.8 (ArC), 129.3 (ArC), 137.1 (ArC), 140.8 (ArC), 142.1 (ArC), 145.4 (ArC), 145.5 (ArC), 146.3 (ArC), 149.4 (ArC), 151.2 (C═C), 152.3 (2×ArC), 160.8 (C═O)

MS: calculated 524.1680, found 525.1766 (M+H⁺), 547.1586 (M+Na⁺).

νₘₐₓ (DCM)/cm⁻¹: 2929.5, 2854.2, 1714.6

### Synthesis of Intermediate 2-methoxy-5-(6,7,8-trimethoxy-2-oxochromen-4-yl)phenyl bis[(benzyloxy)methyl]phosphinate 24.1

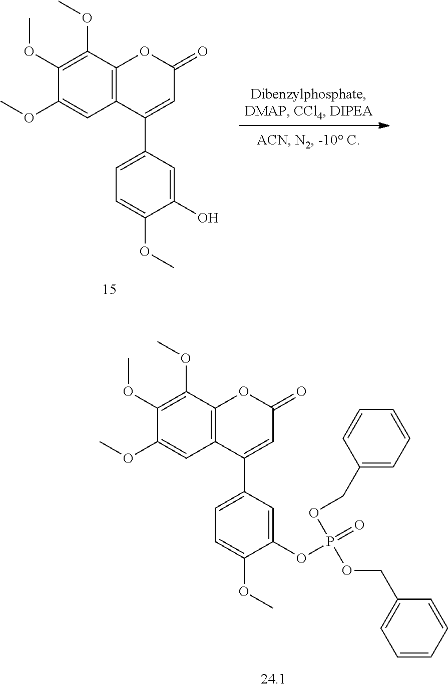 [SMILES: COc1ccc(-c2cc(=O)oc3c(OC)c(OC)c(OC)cc23)cc1O ; "Dibenzylphosphate,DMAP, CCl4, DIPEA" ; COc1ccc(-c2cc(=O)oc3c(OC)c(OC)c(OC)cc23)cc1OP(=O)(OCc1ccccc1)OCc1ccccc1]

Phenol 4-(3-hydroxy-4-methoxyphenyl)-6,7,8-trimethoxychromen-2-one 15 (0.28 g, 0.787 mmoles) and 4-dimethylaminopyridine (5 mg, 44 μmoles) were stirred in acetonitrile (10 mL) under an atmosphere of nitrogen and the reaction cooled to −10° C. Carbon tetrachloride (0.38 mL, 3.93 mmoles) was then added to the mixture, followed by diisopropylethylamine (0.29 mL, 1.65 mmoles). After 30 min, dibenzylphosphate (0.26 mL, 1.18 mmoles) was subsequently added and the reaction left stirring overnight. The reaction was then worked up with monobasic potassium phosphate (50 mL, 0.5 M) and extracted with diethylether (4×50 mL). After concentration under reduced pressure and drying with MgSO₄, the reaction was purified by column chromatography (2:1, hexane:ethyl acetate) to afford phosphate ester 24.1 (0.41 g, 0.69 mmoles, 88%) as a clear oil.

¹H NMR (600 MHz, CHLOROFORM-d) δH: ppm 3.73 (3H, s, OCH₃), 3.88 (3H, s, OCH₃), 4.01 (3H, s, OCH₃), 4.06 (3H, s, OCH₃), 5.17-5.22 (4H, m, 2×CH₂), 6.20 (1H, s, CH═C), 6.71 (1H, s, ArH), 7.06 (1H, d, ArH, J=9.08 Hz), 7.23-7.25 (2H, m, 2×ArH), 7.27-7.36 (10H, m, 10×ArH)

¹³C NMR (151 MHz, CHLOROFORM-d) δC: 55.9 (OCH₃), 56.1 (OCH₃), 61.3 (OCH₃), 61.8 (OCH₃), 69.9 (CH₂), 69.94 (CH₂), 102.9 (ArCH), 112.8 (CH═C), 113.5 (ArCH), 121.7 (ArCH), 125.8 (ArCH), 127.8 (4×ArCH), 127.9 (ArCH), 128.4 (4×ArCH), 128.5 (ArCH), 135.2 (ArC), 135.3 (ArC), 139.5 (ArC), 139.6 (ArC), 141.2 (ArC), 143.2 (ArC), 145.8 (ArC), 149.6 (ArC), 151.7 (ArC), 151.8 (ArC), 153.9 (C═CH), 160.4 (C═O)

MS: calculated 618.1655, found 641.1539 (M+Na⁺).

### Synthesis of disodium 2-methoxy-5-(6,7,8-trimethoxy-2-oxochromen-4-yl)phenyl phosphate 24

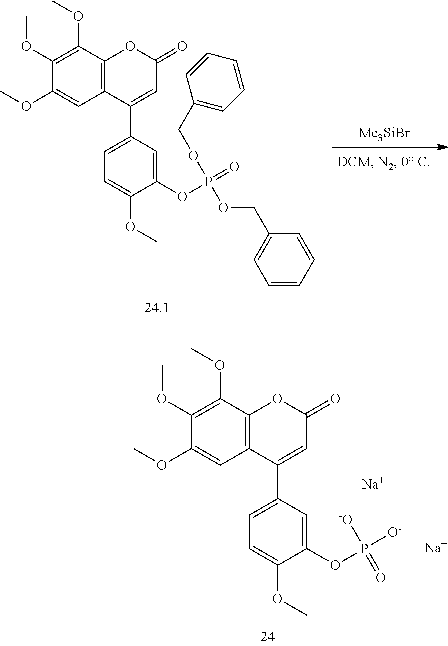

Phosphate ester 2-methoxy-5-(6,7,8-trimethoxy-2-oxochromen-4-yl)phenyl bis[(benzyloxy)methyl]phosphinate 24.1 (0.41 g, 0.69 mmoles) was dissolved in anhydrous DCM under N₂ gas and cooled to 0° C. Bromotrimethyl silane (0.19 mL, 1.45 mmoles) was then added dropwise and the reaction was allowed to stir for 1 h. The DCM was then removed in vacuo, water (50 mL) added to the flask and the reaction allowed stir overnight. The aqueous layers were then separated with diethyl ether (3×50 mL), before the aqueous phase was concentrated in vacuo. When dry, the residue (0.30 g, 0.68 mmoles) was dissolved in MeOH (20 mL) and sodium methoxide (0.07 g, 1.37 mmoles) added. The resulting mixture was allowed stir overnight and evaporated to dryness to afford 24.

¹H NMR (400 MHz, DMSO-d₆) δH: 3.76 (3H, s, OCH₃), 3.81 (3H, s, OCH₃), 3.87 (3H, s, OCH₃), 3.91 (3H, s, OCH₃), 6.27 (1H, s, CH═C), 6.99 (1H, s, ArH), 7.11 (1H, dd, ArH, J=13.25, 8.00 Hz), 7.80 (1H, s, ArH), 8.19 (1H, s, ArH)

MS: calculated 482.0355, found 483.0465 (M+Na⁺).

### Synthesis of Intermediate dibenzyl 2-methoxy-5-(7,8,9-trimethoxy-3-oxo-2H-1-benzoxepin-5-yl)phenyl phosphate 25.1

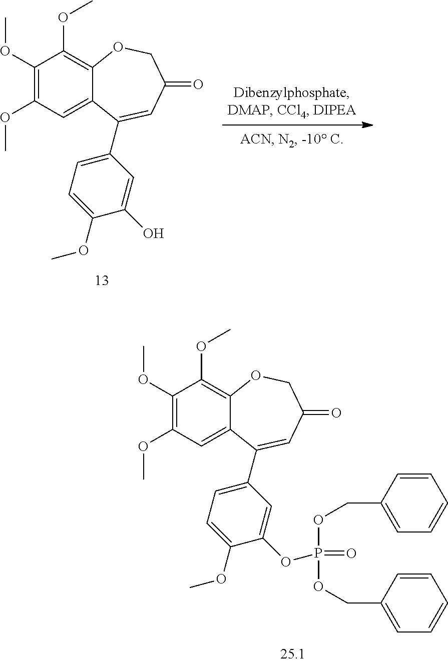 [SMILES: COc1ccc(C2=CC(=O)COc3c2cc(OC)c(OC)c3OC)cc1O ; COc1ccc(C2=CC(=O)COc3c2cc(OC)c(OC)c3OC)cc1OP(=O)(OCc1ccccc1)OCc1ccccc1]

Phenol 5-(3-hydroxy-4-methoxyphenyl)-7,8,9-trimethoxy-2H-1-benzoxepin-3-one 13 (66 mg, 0.177 mmoles) and 4-dimethylaminopyridine (1.1 mg, 9 μmoles) were stirred in acetonitrile (3 mL) under an atmosphere of nitrogen and the reaction cooled to −10° C. Carbon tetrachloride (0.083 mL, 0.0855 mmoles) was then added to the mixture, followed by diisopropylethylamine (0.065 mL, 0.37 mmoles). After 30 min, dibenzylphosphate (0.06 mL, 0.26 mmoles) was subsequently added and the reaction left stirring overnight. The reaction was then worked up with monobasic potassium phosphate (50 mL, 0.5 M) and extracted with diethylether (4×50 mL). After concentration under reduced pressure and drying with MgSO4, the reaction was purified by column chromatography (2:1 hexane:ethyl acetate) to afford phosphate ester 25.1 (72 mg, 0.114 mmoles, 65%) as a clear oil.

¹H NMR (400 MHz, CHLOROFORM-d) δH: 3.6333 (3H, s, OCH₃), 3.8717 (3H, s, OCH₃), 3.9871 (3H, s, OCH₃), 4.0184 (3H, s, OCH₃), 4.6519 (2H, s, OCH2C═O), 5.1750 (2H, s, POCH2), 5.1964 (2H, s, POCH2), 6.3015 (1H, s, CH═C), 6.4131 (1H, s, ArH), 6.9670 (1H, d, ArH, J=8.53 Hz), 7.0868 (1H, t, ArH, J=3.25 Hz), 7.2166 (1H, d, ArH, J=7.53 Hz), 7.3414 (10H, s, 10×ArH)

¹³C NMR (101 MHz, CHLOROFORM-d) δC: 55.52 (OCH₃), 55.75 (OCH₃), 60.85 (OCH₃), 61.46 (OCH₃), 69.49 (POCH2), 69.55 (POCH2), 80.67 (CH2C═O), 109.61 (CH═C), 111.80 (ArCH), 122.26 (ArCH), 125.23 (ArCH), 126.51 (ArCH), 127.46 (4×ArCH), 128.00 (ArC), 128.13 (4×ArCH), 128.18 (2×ArCH), 133.51 (ArC), 135.02 (ArC), 134.99 (ArC), 138.80 (ArC), 144.18 (ArC), 144.79 (ArC), 146.98 (ArC), 148.83 (ArC), 150.24 (ArC), 151.33 (C═CH), 199.95 (C═O)

MS: calculated 632.1811, found 633.1891.

### Synthesis of 2-methoxy-5-(7,8,9-trimethoxy-3-oxo-2H-1-benzoxepin-5-yl)phenyl disodium phosphate 25

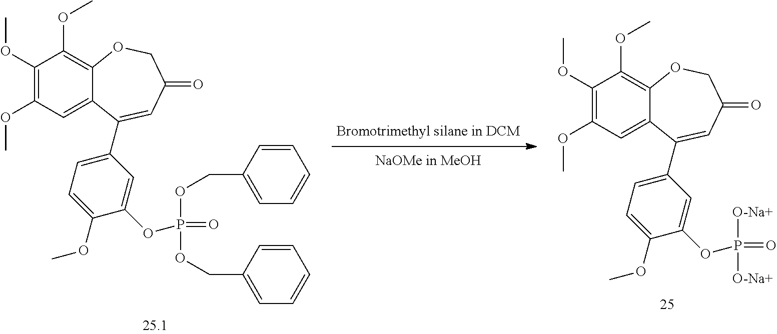

Phosphate 25.1 (72 mg, 0.114 mmoles) was dissolved in anhydrous DCM and cooled to 0° C. Bromotrimethyl silane (0.031 mL, 0.24 mmoles) was then added dropwise and the reaction was allowed to stir for 1 h. The DCM was then removed in vacuo, water (20 mL) added to the flask and the reaction allowed stir overnight. The aqueous layers were then separated with diethyl ether (3×30 mL), before the aqueous phase was concentrated in vacuo. When dry, the residue was dissolved in MeOH (20 mL) and sodium methoxide (11 mg, 0.22 mmoles) added. The resulting mixture was allowed stir overnight, evaporated to dryness to afford the disodium phosphate 25.

### Synthesis of ({[(3E)-5-(3-hydroxy-4-methoxyphenyl)-7,8,9-trimethoxy-2H-1-benzoxepin-3-ylidene]amino}oxy)acetic acid 26.1

### Step 1

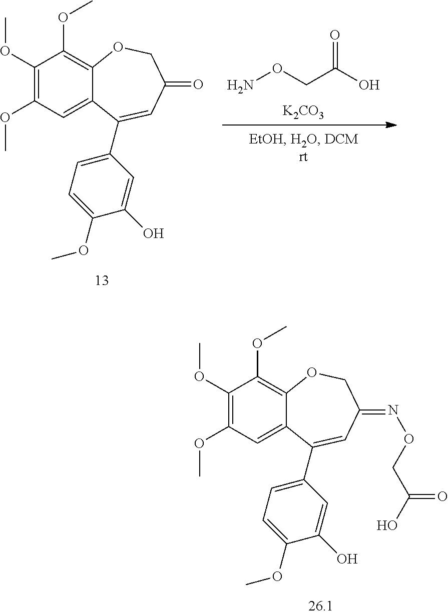

Phenol 13 (36 mg, 0.097 mmoles), sodium acetate (13 mg, 0.15 mmoles), and O-carboxymethyl hydroxylamine hemihydrochloride (12 mg, 0.11 mmoles) were stirred overnight in EtOH:Water:DCM (8:2:1, 5.5 mL) at room temperature. The reaction was then quenched with aq. HCl (20 mL, 1 M) and extracted with diethyl ether (3×30 mL) to give carboxylic acid 26.1 (30 mg, 0.067 mmoles, 70%) as a clear residue.

¹H NMR (600 MHz, CHLOROFORM-d) δH: 3.61 (3H, s, OCH₃, minor isomer), 3.64 (3H, s, OCH₃, major isomer), 3.95 (3H, s, OCH₃, major isomer), 3.96 (3H, s, OCH₃, major isomer), 3.96 (3H, s, OCH₃, minor isomer), 3.98 (3H, s, OCH₃, minor isomer), 3.99 (3H, s, OCH₃, minor isomer), 4.01 (3H, s, OCH₃, major isomer), 4.72 (4H, s, CH₂, 1×CH₂ major isomer, 1×CH₂ minor isomer), 4.73 (2H, s, CH₂, minor isomer), 5.14 (2H, s, CH₂, major isomer), 6.30 (1H, s, ArH, major isomer), 6.37 (1H, s, ArH, minor isomer), 6.55 (1H, s, CH═C, major isomer), 6.84-6.91 (4H, m, 2×major isomer ArH, 2×minor isomer ArH), 6.96 (1H, s, ArH, minor isomer), 6.97 (1H, s, ArH, major isomer), 7.06 (1H, s, CH═C, minor isomer).

¹³C NMR (101 MHz, CHLOROFORM-d) δC: 51.5 (OCH₃, major), 51.6 (OCH₃, minor), 55.7 (OCH₃, minor), 55.8 (OCH₃, major), 60.8 (OCH₃, major), 60.9 (OCH₃, minor), 61.4 (OCH₃, minor), 61.5 (OCH₃, major), 69.8 (CH₂, minor), 70.1 (CH₂, major), 72.0 (CH₂, major), 74.1 (CH₂, minor), 108.8 (ArCH, major), 109.6 (ArCH, minor), 109.7 (ArCH, major), 110.5 (ArCH, minor), 114.9 (ArCH, major), 115.3 (ArCH, minor), 116.2 (CH═C, minor), 120.6 (ArCH, major), 121.0 (ArCH, minor), 122.5 (CH═C, major), 124.7 (ArCH, minor), 126.8 (ArCH, major), 135.4 (ArC, major), 136.4 (ArC, minor), 142.8 (ArC, major), 143.4 (ArC, minor), 143.8 (ArC, major), 144.2 (ArC, minor), 144.66 (ArC, minor), 144.74 (ArC, major), 145.0 (ArC, major), 145.5 (ArC, minor), 146.2 (ArC, major), 147.8 (ArC, minor), 148.6 (ArC, minor), 148.7 (ArC, major), 154.1 (2×C═CH, both isomers), 160.4 (2×C═N, both isomers), 174.0 (C═O, major), 174.1 (C═O, minor)

MS: calculated 445.1373, found 446.1436 (M+H⁺).

### Step 2: Synthesis of Intermediate pentafluorophenyl 2-({[(3E)-5-(3-hydroxy-4-methoxyphenyl)-7,8,9-trimethoxy-2H-1-benzoxepin-3-ylidene]amino}oxy)acetate 26.2

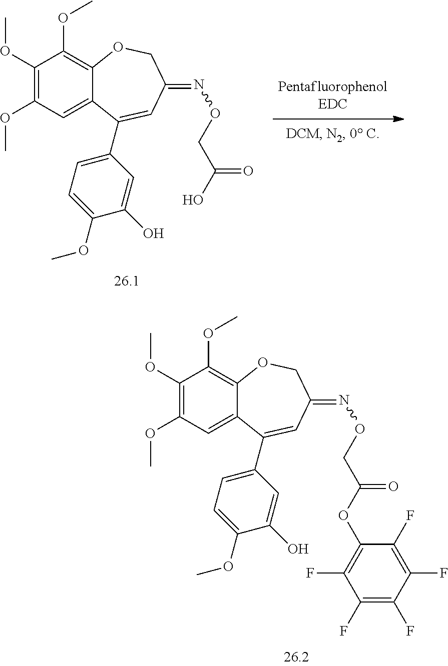 [SMILES: COc1ccc(C2=CC(=NOCC(=O)O)COc3c2cc(OC)c(OC)c3OC)cc1O ; COc1ccc(C2=CC(=NOCC(=O)Oc3c(F)c(F)c(F)c(F)c3F)COc3c2cc(OC)c(OC)c3OC)cc1O]

Carboxylic acid 26.1 (24 mg, 0.054 mmoles) was dissolved in anhydrous DCM (2 mL) under nitrogen gas and cooled to 0° C. To this was added sequentially; pentafluorophenol (9 mg, 0.056 mmoles) in dry DCM (1 mL) and N-(3-dimethylaminopropyl)-N′-ethylcarbodiimide (9.3 mg, 0.056 mmoles) in dry DCM:DMF (1:0.5 mL). The reaction was allowed stir for 1 h when the reaction was quenched with water (20 mL) and extracted with diethyl ether (3×30 mL). The resultant crude compound was purified via column chromatography (3:1, hexane:ethyl acetate) to give pentafluorophenyl ester 26.2 (26 mg, 0.043 mmoles, 79%) as a clear oil.

¹H NMR (600 MHz, CHLOROFORM-d) δH: 3.61 (3H, s, OCH₃, minor isomer), 3.64 (3H, s, OCH₃, major isomer), 3.95 (3H, s, OCH₃, major isomer), 3.962 (3H, s, OCH₃, major isomer), 3.966 (3H, s, OCH₃, minor), 3.98 (3H, s, OCH₃, minor isomer), 3.99 (3H, s, OCH₃, minor isomer), 4.01 (3H, s, OCH₃, major isomer), 4.76 (2H, s, CH₂, minor isomer), 5.01 (4H, s, 1×CH₂ major isomer, 1×CH₂ minor isomer), 5.16 (2H, s, CH₂, major isomer), 5.64 (1H, s, OH, major isomer), 5.67 (1H, s, OH, minor isomer), 6.30 (1H, s, ArH, major isomer), 6.37 (1H, s, ArH, minor isomer), 6.55 (1H, s, CH═C, major isomer), 6.84-6.91 (4H, m, 2×major isomer ArH, 2×minor isomer ArH), 6.96 (1H, d, ArH, J=1.88 Hz, minor isomer), 6.97 (1H, d, ArH J=2.26 Hz, major isomer), 7.08 (1H, s, CH═C, minor isomer)

¹³C NMR (151 MHz, CHLOROFORM-d) δC, 55.83 (OCH₃, major), 55.86 (OCH₃, minor), 56.06 (OCH₃, minor), 56.09 (OCH₃, major), 61.12 (OCH₃, major), 61.15 (OCH₃, minor), 61.70 (OCH₃, major), 61.73 (OCH₃, minor), 69.8 (CH₂, minor), 70.1 (CH₂, major), 72.2 (CH₂, major), 74.4 (CH₂, minor), 109.2 (ArCH, major), 109.9 (ArCH, minor), 110.0 (ArCH, major), 110.9 (ArCH, minor), 115.2 (ArCH, major), 115.6 (ArCH, minor), 116.4 (ArCH, minor), 120.9 (ArCH, major), 121.3 (ArCH, minor), 122.7 (ArCH, major), 125.01 (ArC, minor), 127.04 (ArC, major), 135.7 (ArC, major), 136.8 (ArC, minor), 137.0 (4×ArCF, both isomers), 138.6 (4×ArCF, both isomers), 140.3 (2×ArCF, both isomers), 143.2 (ArC, major), 143.9 (ArC, minor), 144.3 (ArC, major), 144.6 (ArC, minor), 145.0 (ArC, minor), 145.1 (ArC, major), 145.4 (ArC, major), 145.9 (ArC, minor), 146.55 (ArC, major), 146.6 (ArC, major), 148.2 (ArC, minor), 148.9 (ArC, minor), 149.1 (ArC, major), 154.8 (C═CH, major), 154.8 (C═CH, minor), 161.1 (2×C═N), 165.5 (C═O, major), 165.8 (C═O, minor)

¹⁹F NMR (376 MHz, CHLOROFORM-d) δF: −169 (1F, m, minor), −164 (2F, m, minor), −164 (2F, m, minor), −162 (2F, m, major), −158 (1F, m, major), −153 (2F, d, J=18.35 Hz, major)

MS: calculated 611.1215, found 612.1307 (M+H⁺), 634.1125 (M+Na⁺).

### Step 3: Synthesis of N-hydroxy-2-({[(3E)-5-(3-hydroxy-4-methoxyphenyl)-7,8,9-trimethoxy-2H-1-benzoxepin-3-ylidene]amino}oxy)acetamide 26

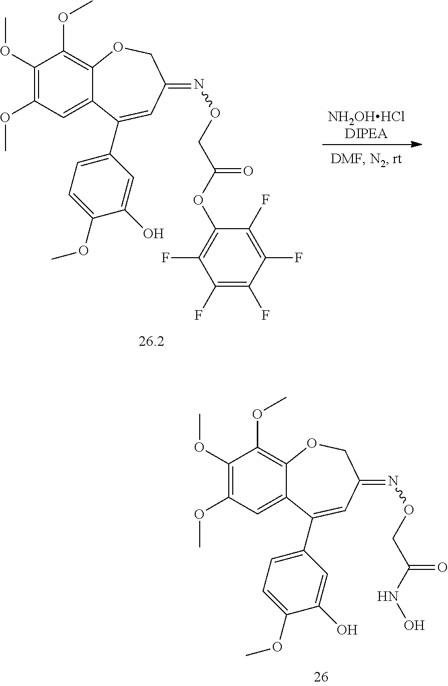 [SMILES: COc1ccc(C2=CC(=NOCC(=O)Oc3c(F)c(F)c(F)c(F)c3F)COc3c2cc(OC)c(OC)c3OC)cc1O ; "NH2OH·HClDIPEA" ; COc1ccc(C2=CC(=NOCC(=O)NO)COc3c2cc(OC)c(OC)c3OC)cc1O]

To a stirred solution of pentafluorophenyl ester 26.2 (20 mg, 0.033 mmoles) in dry DMF (1 mL), under an atmosphere of nitrogen was added hydroxylamine hydrochloride (2.5 mg, 0.036 mmoles) in dry DMF (0.5 mL) and neat diisopropylethylamine (4.6 mg, 0.036 mmoles). The reaction was stirred for 5 min before the reaction was quenched with water (20 mL) and extracted with diethyl ether (3×30 mL) to afford hydroxamic acid 26 (10 mg, 0.022 mmoles, 66%) as a yellow oil.

¹H NMR (600 MHz, CHLOROFORM-d) δH: 3.62 (3H, s, OCH₃, minor isomer), 3.64 (3H, s, OCH₃, major isomer), 3.96 (6H, s, 2×OCH₃, 2×major isomer), 3.97 (3H, s, OCH₃, minor isomer), 3.99 (6H, s, 2×OCH₃, 2×minor isomer), 4.01 (3H, s, OCH₃, major isomer), 4.71 (2H, s, CH₂, minor isomer), 4.73 (4H, s, 2×CH₂, 1×major isomer, 1×minor isomer), 5.09 (2H, s, CH₂, major isomer), 5.69 (1H, br. s, OH), 6.31 (1H, s, ArH, major isomer), 6.37 (1H, s, ArH, minor isomer), 6.52 (1H, s, CH═C, major isomer), 6.83-6.91 (4H, m, 4×ArH, 2×major isomer, 2×minor isomer), 6.94 (1H, s, ArH, minor isomer), 6.96 (2H, s, 1×ArH, major isomer, 1×CH═C, minor isomer), 8.05 (1H, br. s, NH), 8.78 (1H, br. s, OH)

¹³C NMR (151 MHz, CHLOROFORM-d) δC: 55.8 (OCH₃, major), 55.9 (OCH₃, minor), 56.1 (OCH₃, minor, 56.1 (OCH₃, major), 61.1 (OCH₃, major), 61.2 (OCH₃, minor), 61.7 (OCH₃, major), 61.8 (OCH₃, minor), 71.9 (CH₂, minor), 72.0 (CH₂, major), 72.2 (CH₂, minor), 74.1 (CH₂, major), 109.2 (ArH, major), 110.0 (ArH, minor), 110.1 (ArH, major), 111.0 (ArH, minor), 115.2 (ArH, major), 115.6 (ArH, minor), 115.7 (CH═C, minor), 120.9 (ArH, major), 121.2 (ArH, minor), 122.3 (CH═C, major), 124.7 (ArC, minor), 126.9 (ArC, major), 135.5 (ArH, major), 136.6 (ArH, minor), 143.3 (ArC, major), 144.1 (ArC, minor), 144.6 (ArC, minor), 144.9 (ArC, minor), 145.1 (ArC, minor), 145.2 (ArC, major), 146.5 (ArC, major), 146.7 (ArC, major), 146.9 (ArC, minor), 148.2 (ArC, major), 149.0 (ArC, minor), 149.2 (ArC, major), 155.1 (2×C═CH, both isomers), 161.5 (2×C═N, both isomers), 166.7 (C═O, major) 167.0 (C═O, minor).

MS: calculated 460.1482, found 461.1544 (M+H⁺).

### Synthesis of hydroxamic acid 27

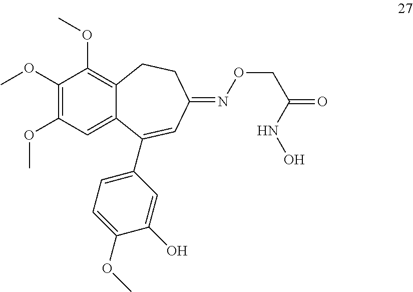 [SMILES: COc1ccc(C2=C/C(=N/OCC(=O)NO)CCc3c2cc(OC)c(OC)c3OC)cc1O]

Synthesis of (E)-2-(((9-(3-((tert-butyldimethylsilyl)oxy)-4-methoxyphenyl)-2,3,4-trimethoxy-5H-benzo[7]annulen-7(6H)-ylidene)amino)oxy)acetic acid 27.1.

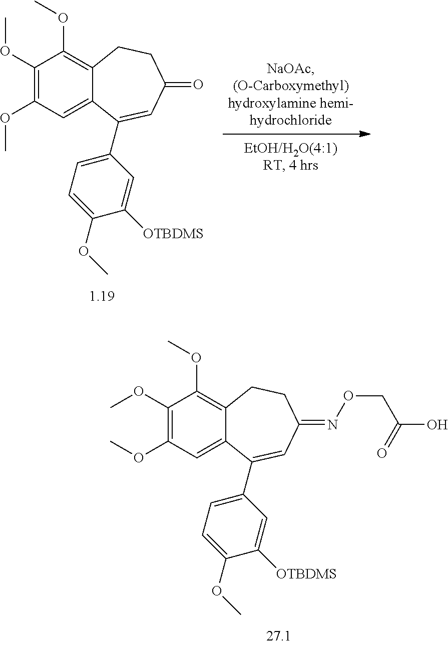 [SMILES: COc1ccc(C2=CC(=O)CCc3c2cc(OC)c(OC)c3OC)cc1O[Si](C)(C)C(C)(C)C ; "NaOAc,(O-Carboxymethyl)hydroxylamine hemi-hydrochloride" ; COc1ccc(C2=C/C(=N/OCC(=O)O)CCc3c2cc(OC)c(OC)c3OC)cc1O[Si](C)(C)C(C)(C)C]

To a stirred solution of 9-(3-((tert-butyldimethylsilyl)oxy)-4-methoxyphenyl)-2,3,4-trimethoxy-5H-benzo[7]annulen-7(6H)-one 1.19 (0.07 g, 0.14 mmol) in EtOH (4 mL) and H₂O (1 mL), was added (O-carboxymethyl)hydroxylamine hemi-hydrochloride (0.02 g, 0.16 mmol) and NaOAc (0.02 g, 0.23 mmol). The resultant mixture was stirred at room temperature for 4 hrs, until complete consumption of the starting material was observed using TLC (hexane/ethyl acetate 1:1). The mixture was diluted in water (10 mL) and the product was extracted using ether (3×15 mL). The product 27.1 was isolated as a mixture of isomers by column chromatography using ethyl acetate as the mobile phase, in the form of a yellow oil.

Yield: (0.06 g, 0.11 mmol, 76%).

¹H NMR (CDCl₃, 400 MHz) δ_{H} ppm: 0.16 (s, 6H, 2×CH₃), 0.99 (s, 9H, ^{t}Bu), 2.75-3.05 (m, 4H, 2×CH₂), 3.61 (s, 3H, OCH₃), 3.86 (s, 3H, OCH₃), 3.89 (s, 3H, OCH₃), 3.91 (s, 3H, OCH₃), 4.71 (d, 2H, OCH₂), 6.33 (s, 1H), 6.50 (s, 1H), 6.81-6.85 (m, 2H), 6.93-6.96 (m, 1H).

¹³C NMR (CDCl₃, 100.71 MHz) δ_{c} ppm: −5.0 (CH₃), 0.67 (CH₃), 14.8 (CH₃), 18.0 (Q), 20.7 (CH₂), 21.4 (CH₂), 21.4 (CH₂), 25.3 (CH₃), 29.8 (CH₃), 32.5 (CH₂), 36.1 (CH₂), 54.9 (CH₃), 55.4 (CH₃), 60.5 (CH₃), 60.9 (CH₃), 69.8 (CH₂), 117.0 (CH), 121.1 (CH), 121.4 (CH), 122.0 (CH), 122.3 (CH), 122.6 (CH), 128.0 (CH), 128.7 (CH), 132.9 (CH), 133.0 (CH), 135.0 (CH), 136.4 (CH), 141.8 (CH), 143.9 (Q), 146.0 (Q), 146.6 (Q), 149.6 (Q), 150.3 (Q), 150.4 (Q), 150.5 (Q), 150.8 (Q), 161.8 (Q), 173.8 (Q, C═O).

### Synthesis of the pentafluorophenol ester of (E)-2-(((9-(3-((tert-butyldimethylsilyl)oxy)-4-methoxyphenyl)-2,3,4-trimethoxy-5H-benzo[7]annulen-7(6H)-ylidene)amino)oxy)acetic acid 27.2

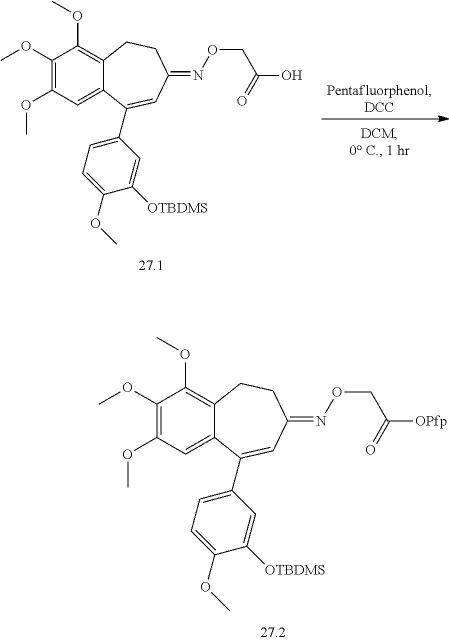

To a stirred solution of (E)-2-(((9-(3-((tert-butyldimethylsilyl)oxy)-4-methoxyphenyl)-2,3,4-trimethoxy-5H-benzo[7]annulen-7(6H)-ylidene)amino)oxy)acetic acid 27.1 (0.44 g, 0.79 mmol) in DCM (3 mL) under an atmosphere of N₂ at 0° C. was added a solution of pentafluorophenol (0.15 g, 0.79 mmol) in DCM (1.5 mL). This was followed by the subsequent addition of DCC (0.16 g, 0.79 mmol) in DCM (1.5 mL). The solution was stirred for 1 hr, after which time the dicyclohexylurea by-product was removed via filtration. The mixture was then washed between water (20 mL) and ether (2×20 mL) to yield the product in crude form. Purification using column chromatography yielded the product in a mixture of syn- and anti-isomers of 27.2 as a colourless oil.

Yield: 0.51 g (0.70 mmol, 89%)

¹H NMR (CDCl₃, 400 MHz) δ_{H} ppm: 0.17 (s, 6H, 2×CH₃), 1.00 (s, 9H, ^{t}Bu), 2.75-2.83 (m, 2H, CH₂), 2.93-3.08 (m, 2H, CH₂), 3.61 (s, 2H, OCH₃ major isomer), 3.62 (s, 1H, OCH₃ minor isomer), 3.91 (s, 2H, OCH₃ major isomer), 3.92 (s, 2H, OCH₃ major isomer), 3.93 (s, 1H, OCH₃ minor isomer), 3.94 (s, 1H, OCH₃ minor isomer), 4.17 (s, 1H, CH₂ minor isomer), 5.01 (s, 1H, CH₂ major isomer), 6.31 (s, 1H, ArHmajor isomer), 6.36 (s, 1H, ArHminor isomer), 6.52 (s, 1H, ArHmajor isomer), 6.81-6.87 (m, 2H), 6.93-6.98 (m, 1H), 7.03 (s, 1H, minor isomer).

¹³C NMR (CDCl₃, 100.71 MHz) δ_{c} ppm: 0.6 (CH₃), 18.0 (Q), 20.7 (CH₂ major isomer), 21.40 (CH₂ minor isomer), 25.2 (CH₃), 32.5 (CH₂ major isomer), 36.1 (CH₂ minor isomer), 55.0 (CH₃), 55.4 (CH₃), 60.4 (CH₃), 61.0 (CH₃), 69.3 (CH₂ minor isomer), 69.5 (CH₂ major isomer), 110.0 (CH), 110.9 (CH major isomer), 111.0 (CH minor isomer), 117.1 (CH), 121.1 (CH major isomer), 121.5 (CH minor isomer), 122.0 (CH major isomer), 122.3 (CH minor isomer), 122.6 (CH), 128.0 (Q), 128.7 (Q), 132.9 (Q), 133.0 (Q), 136.0 (Q), 136.4 (Q), 141.8 (Q), 142.1 (Q), 144.0 (Q), 146.0 (Q), 146.3 (Q), 149.5 (Q minor isomer), 149.6 (Q major isomer), 150.3 (minor isomer), 150.4 (Q major isomer), 150.5 (Q major isomer), 150.8 (Q minor isomer), 158.4 (Q), 161.9 (Q, C═N), 165.7 (Q, C═O major isomer), 165.8 (Q, C═O minor isomer).

### Synthesis of (E)-2-a(9-(3-((tert-butyldimethylsilyl)oxy)-4-methoxyphenyl)-2,3,4-trimethoxy-5H-benzo[7]annulen-7(6H)-ylidene)amino)oxy)-N-hydroxyacetamide 27.3

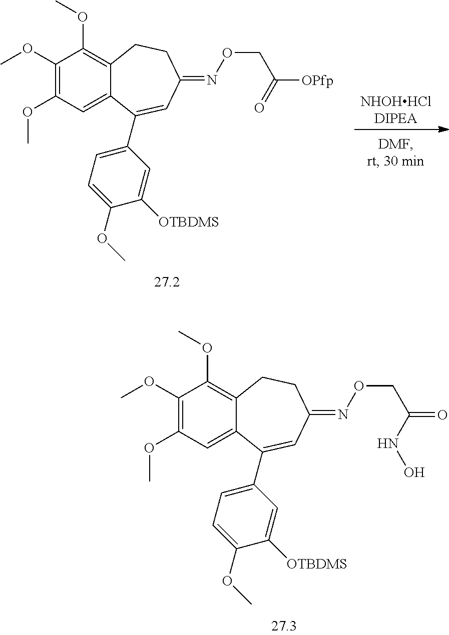

A solution of the pentafluorophenol ester 27.2 (0.0997 g, 0.138 mmol), hydroxylamine hydrochloride (0.105 g, 0.151 mmol) and DIPEA (0.019 g, 0.151 mmol) in DMF (2 mL) under N₂ was stirred at room temperature for 30 min. At this time, the product was extracted by washing between 0.5 M HCl (15 mL) and ether (3×15 mL). The combined ether extracts were washed with water (15 mL) and dried over MgSO4. Following concentration in vacuo, the product 27.3 was purified as a mixture of syn- and anti-isomers using column chromatography with ethyl acetate as the mobile phase as an oil with a yellowish tinge.

Yield: 0.0419 g (0.074 mmol, 53.5%)

¹H NMR (CDCl₃, 400 MHz) δ_{H} ppm: 0.17 (s, 6H, 2×CH₃), 1.00 (s, 9H, ^{t}Bu), 2.72-2.93 (m, 2H, CH₂), 2.96-3.08 (m, 2H, CH₂), 3.63 (s, 3H, OCH₃), 3.86 (s, 2H, OCH₃ major isomer), 3.87 (s, 1H, OCH₃ minor isomer), 3.91 (s, 1H, OCH₃ minor isomer), 3.93 (s, 2H, OCH₃ major isomer), 3.93 (s, 2H, OCH₃ major isomer), 3.94 (s, 1H, OCH₃ minor isomer), 4.67 (s, 1H, CH₂ minor isomer), 4.70 (s, 1H, CH₂ major isomer), 6.32 (s, 11-1, ArH major isomer), 6.37 (s, 1H, ArH minor isomer), 6.49 (s, 1H, ArH), 6.80-6.89 (m, 2H), 6.94 (s, 1H, ArH major isomer), 6.96 (s, 1H, ArH minor isomer), 8.72 (br s, 1′-1, NH).

¹³C NMR (CDCl₃, 100.71 MHz) θ_{c} ppm: −4.5 (CH₃OSi), 1.0 (CH₃OSi), 18.5 (Q), 21.1 (CH₂ major isomer), 22.7 (CH₂ minor isomer), 25.7 (^{t}Bu), 29.7 (CH₂ major isomer), 33.2 (CH₂ minor isomer), 55.4 (CH₃), 55.9 (CH₃), 60.9 (CH₃), 61.4 (CH₃), 71.9 (CH₂), 109.1 (CH minor isomer), 110.6 (CH major isomer), 111.5 (CH), 121.5 (CH), 121.8 (CH), 122.4 (CH), 122.6 (CH), 128.1 (Q), 129.1 (Q), 133.0 (Q), 133.3 (Q), 136.2 (Q), 136.6 (Q), 142.4 (Q), 144.6 (Q), 147.0 (Q), 147.6 (Q), 150.1 (Q), 150.9 (Q), 151.1 (Q), 151.1 (Q), 151.4 (Q), 154.5 (Q, C═N), 162.8 (Q, C═O).

MS (−ESI): Calculated Mass 572.7220. Found 571.2495 (M−H)⁻.

### Synthesis of (E)-N-hydroxy-2-(((9-(3-hydroxy-4-methoxyphenyl)-2,3,4-trimethoxy-5H-benzo[7]annulen-7(6H)-ylidene)amino)oxy)acetamide 27.4

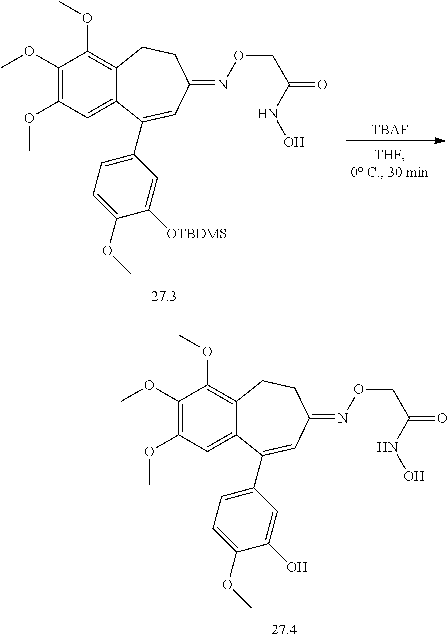

To a stirred solution of (E)-2-(((9-(3-((tert-butyldimethylsilyl)oxy)-4-methoxyphenyl)-2,3,4-trimethoxy-5H-benzo[7]annulen-7(6H)-ylidene)amino)oxy)-N-hydroxyacetamide 27.3 (0.0419 g, 0.0732 mmol) in THF (1 mL) at 0° C. under an atmosphere of N₂, was added a 1M solution of TBAF (0.08 mL, 0.08 mmol). The mixture was stirred at this temperature for 30 min. TLC analysis showed that the starting material had been consumed. The solvent was removed by the bubbling of N₂ through it. The product 27.4 was then obtained in a mixture of syn and anti isomers as a yellow oil using column chromatography (hexane/ethyl acetate 2:1).

Yield: 0.029 g (0.063 mmol, 86.4%).

¹H NMR (CDCl₃, 400 MHz) δ_{H} ppm: 2.76-2.78 (m, 1H, CH₂ ring minor isomer), 2.83-3.00 (m, 3H, mixture of CH₂ rings), 3.63 (s, 2H, OCH₃ major isomer), 3.64 (s, 1H, OCH₃ minor isomer), 3.89 (s, 2H, OCH₃ minor isomer), 3.91 (s, 2H, OCH₃ major isomer), 3.92 (s, 2H, OCH₃ minor isomer), 3.93 (s, 2H, OCH₃ major isomer), 3.94 (s, 2H, OCH₃ major isomer), 3.95 (s, 2H, OCH₃ minor isomer), 4.68 (s, 2H, CH₂ major isomer), 4.87 (s, 2H, CH₂ minor isomer), 6.34 (s, 1H, ArCH major isomer), 6.53 (s, 1H, ArCH minor isomer), 6.82-6.93 (m, 3H, 3×ArCH).

¹³C NMR (CDCl₃, 100.71 MHz) δ_{c} ppm: 21.1 (CH₂ major isomer), 21.8 (CH₂ minor isomer), 33.3 (CH₂ major isomer), 36.8 (CH₂ minor isomer), 56.0 (2×CH₃), 61.0 (CH₃), 61.5 (CH₃), 69.0 (CH₂ minor isomer), 71.9 (CH₂ major isomer), 110.2 (CH), 110.6 (CH), 111.5 (CH minor isomer), 115.2 (CH major isomer), 115.3 (CH major isomer), 115.4 (CH minor isomer), 120.8 (CH major isomer), 121.1 (CH minor isomer), 122.8 (CH major isomer), 122.9 (CH minor isomer), 123.4 (Q), 128.2 (Q), 129.0 (Q), 133.0 (Q), 133.1 (Q), 133.2 (Q), 136.7 (Q), 137.0 (Q), 142.3 (Q), 142.4 (Q), 142.7 (Q), 145.1 (Q), 145.2 (Q), 146.3 (Q) 146.7 (Q), 146.9 (Q), 147.0 (Q), 150.1 (Q), 151.1 (Q), 159.3 (Q), 162.7 (Q, C═N), 167.6 (Q, C═O)

MS (+ESI): Calculated Mass 458.1689. Found 459.1778 (M+H)⁺.

### 4.4 Synthesis of 9-(3-amino-4-methoxyphenyl)-2,3,4-trimethoxy-5H-benzo[7]annulen-7(6H)-one 28

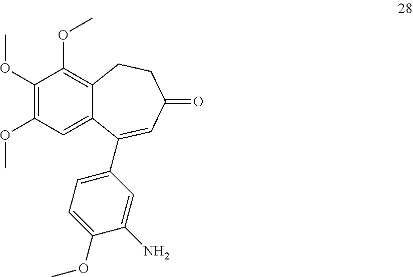 [SMILES: COc1ccc(C2=CC(=O)CCc3c2cc(OC)c(OC)c3OC)cc1N]

### Step 1

### Methylation of Phenol: Synthesis of 4-bromo-1-methoxy-2-nitrobenzene 22.2

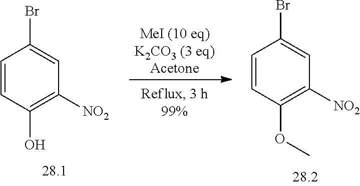

To a stirred solution of 4-bromo-2-nitrophenol 28.1 (5.46 g, 25.04 mmol) in acetone (60 mL) was added potassium carbonate (10.38 g, 75.13 mmol) and iodomethane (15.59 mL, 250.45 mmol). The reaction was heated under reflux for 3 h. The reaction was quenched by the addition of 2M HCl aqueous solution (200 mL) and extracted with diethyl ether. The organic fractions were dried over MgSO₄, filtered and concentrated in vacuo. The product was not purified further and afforded 28.2 as an off white solid (5.78 g, 99%).

¹H NMR (CDCl₃, 400 MHz) δ_{H} ppm: 3.98 (3H, s, OMe), 7.01 (1H, d, J=8.92 Hz, ArH), 7.65 (1H, dd, J₁=2.48 Hz, J₂=9 Hz, ArH), 7.98 (1H, d, J=2.5 Hz, ArH)

¹³C NMR (CDCl₃, 400 MHz) δ_{c} ppm: 56.29 (OMe), 111.35 (ArC), 114.74 (ArCH), 127.89 (ArCH), 136.50 (ArCH), 139.51 (ArC), 151.69 (ArC)

νₘₐₓ (DCM)/cm⁻¹: 3105.13, 2980.33, 2948.81, 2845.25, 1906.06, 1605.52, 1516.15

HRMS: [M+1] calculated 231.9609, found 231.9254, molecular formula (C₇H₇BrNO₃).

Melting Point: 86° C.

### Step 2

### Reduction of Nitro Group: Synthesis of 5-bromo-2-methoxyaniline 28.3

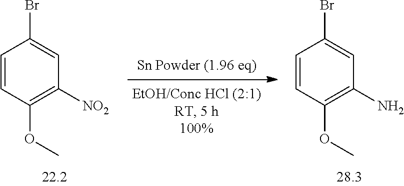

To a stirred solution of 28.2 (0.95 g, 4.09 mmol) in ethanol (30 mL) was added concentrated HCl (15 mL) and tin powder (0.95 g, 8 mmol). The reaction was stirred for 5 h. The solvent was then removed in vacuo and the acid was neutralised by the slow addition of 2.5M NaOH solution (13 mL) at 0° C. The aqueous mixture was then extracted with diethyl ether (3×50 mL). The organic fractions were dried over MgSO₄, filtered and concentrated in vacuo to yield 28.3 as a brown solid (0.86 g, 100%). The product was not purified further.

¹H NMR (CDCl₃, 400 MHz) δ_{H} ppm: 3.85 (3H, s, OMe), 6.65 (1H, dd, J₁=2 Hz, J₂=10.12 Hz, ArH), 6.83 (1H, dd, J₁=2.44 Hz, J₂=8.2 Hz, ArH), 6.85 (1H, s, ArH)

¹³C NMR (CDCl₃, 400 MHz) δ_{c} ppm: 55.18 (ArC), 111.14 (ArCH), 112.75 (ArC), 116.87 (ArCH), 117.72 (ArC), 120.23 (ArCH), 137.15 (ArC), 145.90 (ArC)

vₘₐₓ (DCM)/cm⁻¹: 3460.96, 3370.98, 1611.91, 1573.81

HRMS: calculated 201.9862, found 201.9855, molecular formula (C₇H₉BrNO).

Melting Point: 110° C.

### Step 3

### Boc Protection: Synthesis of tert-butyl (5-bromo-2-methoxyphenyl)carbamate 28.4

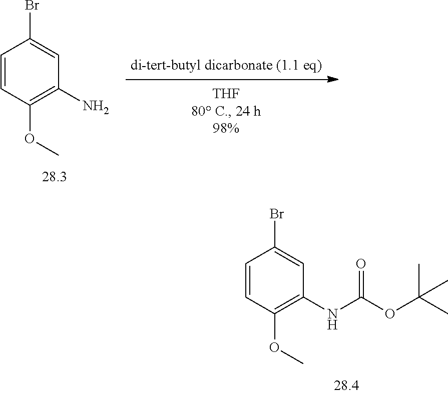

To a stirred solution of 28.3 (0.63 g, 3.12 mmol) in dry THF (5 mL) under an atmosphere of nitrogen was added 1M di-tert-butyl dicarbonate solution in THF (3.43 mL, 3.43 mmol). The reaction was heated under reflux at 80° C. for 24 h. The solvent was removed in vacuo and the resulting residue was purified by purified by flash column chromatography (stationary phase; silica gel 230-400 mesh, mobile phase; 20:1, hexane/ethyl acetate) to yield 28.4 as clear oil (0.92 g, 98%).

¹H NMR (CDCl₃, 400 MHz) δ_{H} ppm: 1.55 (9H, s, C(CH₃)₃), 3.87 (3H, s, OMe), 6.71 (1H, d, J=8.6 Hz, ArH), 7.08 (2H, dd, J₁=2.4H, J₂=8.6 Hz, 2×ArH), 8.30 (1H, s, br, ArNH)

¹³C NMR (CDCl₃, 400 MHz) δ_{c} ppm: 27.85 (C(CH₃)₃, 55.39 (OMe), 80.33 (C(CH₃)₃), 110.67 (ArCH), 113.09 (ArC), 120.17 (ArCH), 124.22 (ArCH), 128.89 (ArC), 145.99 (ArC), 151.95 (C═O)

vₘₐₓ (DCM)/cm⁻¹: 3434.78, 2977.96, 2934.83, 1729.29, 1595.81

HRMS: calculated [M+Na⁺] 324.0211, found 324.0197, molecular formula (C₁₂H₁₆BrNO₃Na).

### Step 4

### Synthesis of Boc protected Boronic Acid: Synthesis of (3-((tert-butoxycarbonyl)amino)-4-methoxyphenyl)boronic acid 28.5

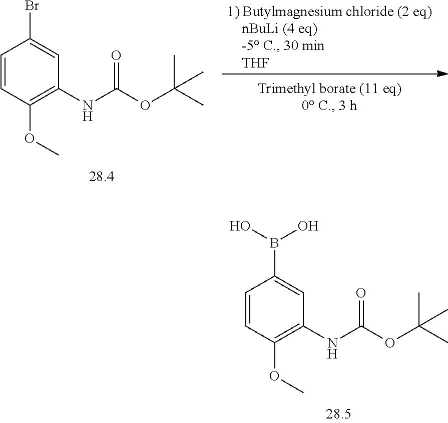

To a stirred 3 necked round bottomed flask containing THF (7 mL) at −5° C. under an atmosphere of nitrogen was added 2M butylmagnesium chloride solution in THF (1.69 mL, 3.38 mmol). After 10 min 2.5M n-butyllithium solution in hexane (2.7 mL, 6.75 mmol) was added dropwise. The solution was stirred at −5° C. for 30 min. A solution of 28.4 (0.51 g, 1.69 mmol) in dry THF (5 mL) was added dropwise and the solution was stirred for 30 min. Whilst maintaining the temperature at −5° C., trimethyl borate (2.07 mL, 18.57 mmol) was added to the reaction mixture in a dropwise manner. The temperature of the reaction was allowed to gradually increase to 0° C. and was left stirring at this temperature for 3 h. The reaction was quenched with ammonium chloride aqueous solution (50 mL) and extracted with diethyl ether (3×50 mL). The organic fractions were dried over MgSO₄, filtered and concentrated in vacuo. It was then attempted to purify the resulting residue by flash column chromatography (stationary phase; silica gel 230-400 mesh, mobile phase; 3:1, hexane/ethyl acetate) to yield 28.5 as part of a crude mixture. The crude product was not purified further but was carried forward to the next step.

### Step 5

Suzuki Coupling: Synthesis of tert-butyl (5-(7-((tert-butyldiphenylsilyl)oxy)-2,3,4-trimethoxy-6,7-dihydro-5H-benzo[7]annulen-9-yl)-2-methoxyphenyl)carbamate 28.6.

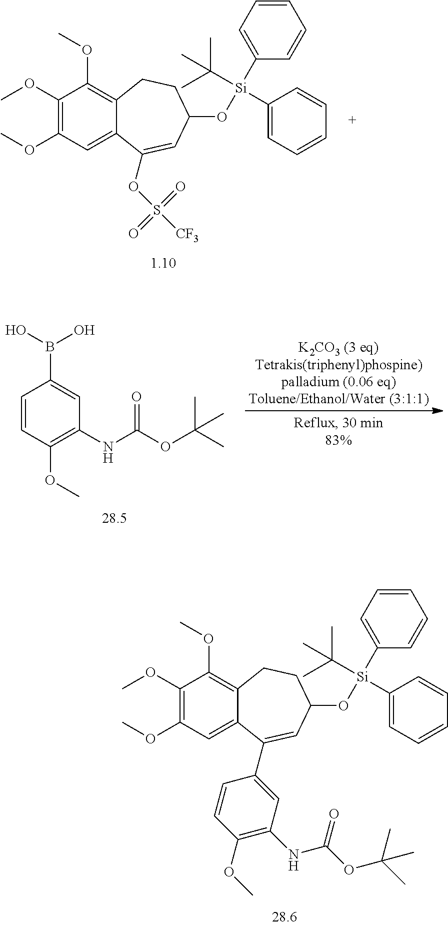

To a stirred solution of 1.10 (0.11 g, 0.17 mmol) in a mixture of toluene, ethanol and water (5 mL; 3:1:1) was added potassium carbonate (0.07 g, 0.52 mmol), (tetrakis(triphenyl)phosphine) palladium (0.01 g, 0.01 mmol) and 28.5 (0.06 g, 0.20 mmol). The reaction was heated under reflux for 30 min after which time it was quenched by the addition of water (50 mL). The reaction mixture was then extracted with diethyl ether (3×50 mL). The organic fractions were dried over MgSO₄, filtered and concentrated in vacuo. The resulting residue was then purified by flash column chromatography (stationary phase; silica gel 230-400 mesh, mobile phase; 10:1, hexane/ethyl acetate) to yield 28.6 as a yellow oil (0.1 g, 83%).

¹H NMR (CDCl₃, 400 MHz) δ_{H} ppm: 1.09 (9H, s, SiC(CH₃)₃), 1.52 (9H, s, COOC(CH₃)₃), 2.18-2.34 (4H, m, 2×CH₂), 3.67 (3H, s, OMe), 3.71 (3H, s, OMe), 3.88 (3H, s, OMe), 3.90 (3H, s, OMe), 4.10-4.16 (1H, m, CHOSi), 6.23 (1H, s, ArH), 6.36 (1H, d, J=4.8 Hz, CH═C), 6.69 (1H, dd, J₁=2.1 Hz, J₂=8.5 Hz, ArH), 7.03 (1H, s, br, ArNH), 7.30 (1H, s, ArH), 7.32-7.40 (6H, m, ArH), 7.62-7.70 (4H, m, ArH)

¹³C NMR (CDCl₃, 400 MHz) δ_{c} ppm: 14.16 (C(CH₃)₃), 19.15 (C(CH₃)₃), 21.86 (CH₂), 26.98 (C(CH₃)₃), 28.35 (C(CH₃)₃), 43.83 (CH₂), 55.75 (CH₃), 56.03 (CH₃), 60.83 (CH₃), 61.53 (CH₃), 71.33 (CH), 108.79 (CH), 109.45 (CH), 118.13 (CH), 122.24 (CH), 127.43 (2×CH), 127.52 (2×CH), 127.76 (QC), 128.01 (QC), 129.41 (CH), 129.44 (CH), 133.02 (CH), 134.24 (QC), 134.36 (QC), 134.41 (QC), 134.46 (QC), 135.74 (2×CH), 135.84 (2×CH), 137.88 (QC), 141.13 (QC), 147.15 (QC), 150.63 (QC), 150.82 (QC), 152.68 (C═O)

vₘₐₓ (DCM)/cm⁻¹: 2931.45, 2856.77, 1730.76, 1589.65, 1527.16

HRMS: calculated 732.3332, found 732.2711, molecular formula (C₄₂H₅₁NO₇SiNa).

### Alternative Approach to Step 4

### Boronic Acid Ester Synthesis: Synthesis of tert-butyl (2-methoxy-5-(4,4,5,5-tetramethyl-1,3,2-dioxaborolan-2-yl)phenyl)carbamate 28.7

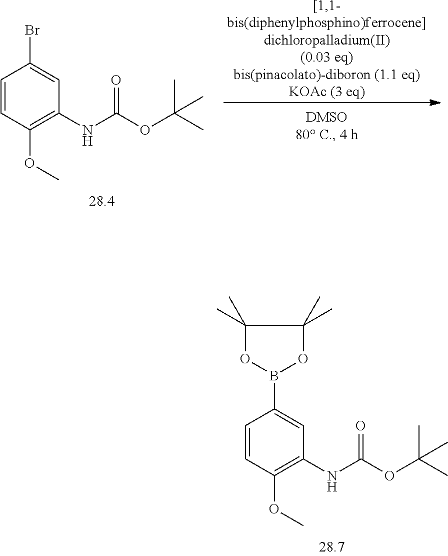

To a dry round bottomed flask containing [1,1′-bis(diphenylphosphino)ferrocene]dichloropalladium(II) (0.01 g, 0.01 mmol), potassium acetate (0.11 g, 1.09 mmol) and bis(pinacolato)-diboron (0.1 g, 0.4 mmol) under an atmosphere of nitrogen was added a solution of 28.4 (0.11 g, 0.36 mmol) in dry DMSO (6 mL). The reaction was then heated under reflux at 80° C. for 4 h. The reaction was quenched with water (25 mL). The reaction mixture was then extracted with diethyl ether (3×50 mL). The organic fractions were dried over MgSO₄, filtered and concentrated in vacuo. The resulting residue was then purified by flash column chromatography (stationary phase; silica gel 230-400 mesh, mobile phase; 10:1, hexane/ethyl acetate) to yield 28.7 a white solid (0.05 g, 40%).

¹H NMR (CDCl₃, 400 MHz) δ_{H} ppm: 1.33 (12H, s, 2×C(CH₃)₂, 1.54 (9H, s, C(CH₃)₃), 3.88 (3H, s, OMe), 6.87 (1H, d, J=8.08 Hz, ArH), 7.06 (1H, s, br ArH), 7.48 (1H, dd, J₁=1.03 Hz, J₂=8.08 Hz, ArH), 8.47 (1H, s, br, NH)

¹³C NMR (CDCl₃, 400 MHz) δ_{c} ppm: 25.04 (2×C(CH₃)₂), 28.39 (C(CH₃)₃), 55.59 (OMe), 80.05 (C(CH₃)₃), 83.58 (2×C(CH₃)₂), 109.23 (ArCH), 124.11 (ArC), 127.52 (ArC), 129.80 (2×ArCH), 150.09 (ArC), 152.50 (C═O)

vₘₐₓ (DCM)/cm⁻¹: 3443.03, 2979.23, 1732.52, 1604.30, 1536.44

HRMS: calculated 372.1958, found 372.1958, molecular formula (C₁₈H₂₈BNNaO₅).

Melting Point: 104° C.

### Alternative Step 6

### Suzuki Coupling of Boronic Ester with Triflate: Alternative Synthesis of tert-butyl (5-(7-((tert-butyldiphenylsilyl)oxy)-2,3,4-trimethoxy-6,7-dihydro-5H-benzo[7]annulen-9-yl)-2-methoxyphenyl)carbamate 28.6.

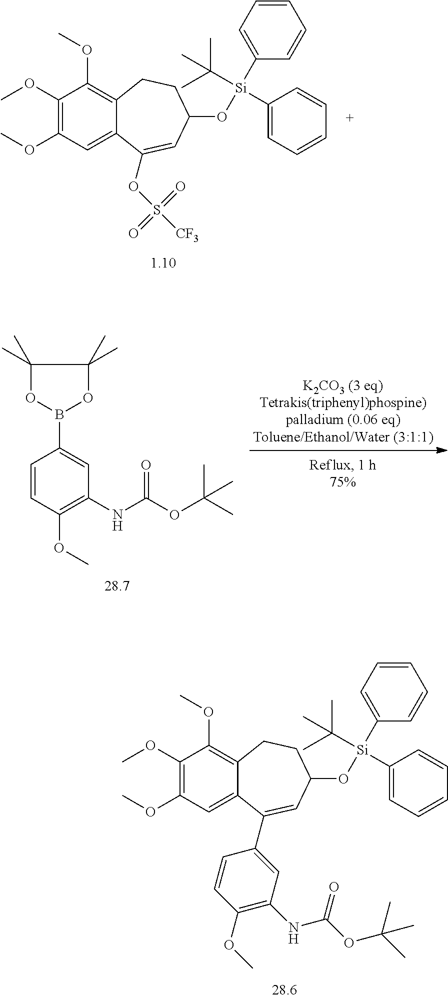

To a stirred solution of 1.10 (0.05 g, 0.07 mmol) in a mixture of toluene, ethanol and water (5 mL; 3:1:1) was added potassium carbonate (0.03 g, 0.22 mmol), (tetrakis(triphenyl)phosphine) palladium (0.005 g, 0.004 mmol) and 22.8 (0.03 g, 0.09 mmol). The reaction was heated under reflux for 1 hour after which time it was quenched by the addition of water (50 mL). The reaction mixture was then extracted with diethyl ether (3×50 mL). The organic fractions were dried over MgSO₄, filtered and concentrated in vacuo. The resulting residue was then purified by flash column chromatography (stationary phase; silica gel 230-400 mesh, mobile phase; 10:1, hexane/ethyl acetate) to yield 22.7 as a yellow oil (0.04 g, 75%).

### Alternative to Step 4 and 5

### One pot biaryl synthesis via in situ boronate formation: Alternative synthesis of tert-butyl (5-(7-((tert-butyldiphenylsilyl)oxy)-2,3,4-trimethoxy-6,7-dihydro-5H-benzo[7]annulen-9-yl)-2-methoxyphenyl)carbamate 28.6

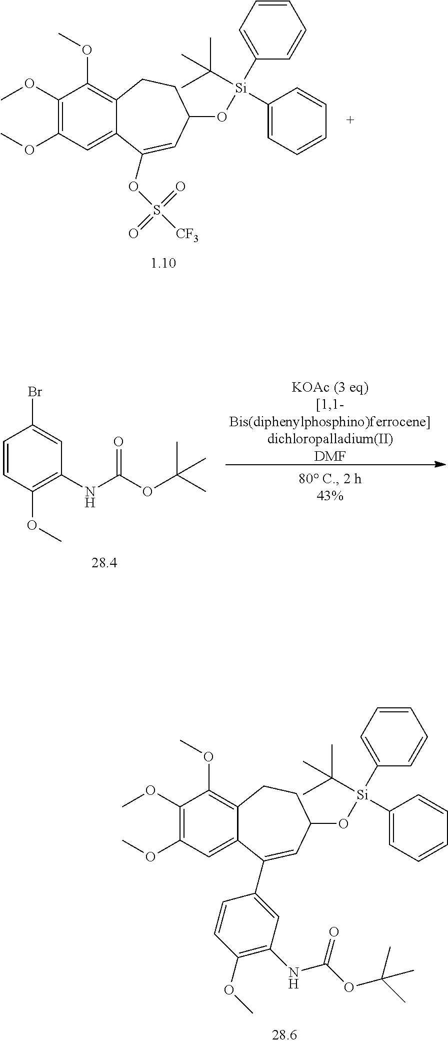

To a clean dry round bottomed flask containing 28.4 (0.2 g, 0.66 mmol) was added bis(pinacolato)-diboron (0.18 g, 0.73 mmol), potassium acetate (0.2 g, 1.99 mmol) and [1,1′-Bis(diphenylphosphino)ferrocene]dichloropalladium(II) (0.015 g, 0.02 mmol). The reaction vessel was flushed with nitrogen and maintained under an atmosphere of nitrogen. Dry DMF (4 mL) was added to the mixture and the reaction was heated under reflux at 80° C. for 2 h. A solution of 1.10 (0.84 g, 1.32 mmol) in dry DMF (2 mL) and 2M sodium carbonate solution (2 mL) were added to the reaction mixture and the reaction was heated for a further 30 min at 80° C. The reaction was quenched by the addition of water (50 mL) and extracted with diethyl ether (3×50 mL). The organic fractions were dried over MgSO₄, filtered and concentrated in vacuo. The resulting black residue was then purified by flash column chromatography (stationary phase; silica gel 230-400 mesh, mobile phase; 4:1, hexane/ethyl acetate) to yield 28.6 as a yellow oil (0.2 g, 43%).

### Step 7

### Silyl Deprotection: Synthesis of tert-butyl (5-(7-hydroxy-2,3,4-trimethoxy-6,7-dihydro-5H-benzo[7]annulen-9-yl)-2-methoxyphenyl)carbamate 28.9)

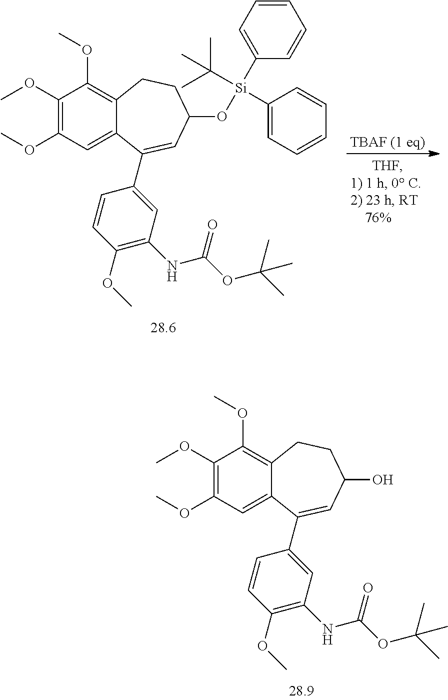 [SMILES: COc1ccc(C2=CC(O[Si](c3ccccc3)(c3ccccc3)C(C)(C)C)CCc3c2cc(OC)c(OC)c3OC)cc1NC(=O)OC(C)(C)C ; "TBAF (1 eq)" ; COc1ccc(C2=CC(O)CCc3c2cc(OC)c(OC)c3OC)cc1NC(=O)OC(C)(C)C]

To a stirred solution of 28.6 (0.04 g, 0.06 mmol) in THF (1 mL) at 0° C. was added 1M tetrabutylammonium fluoride solution (0.06 mL, 0.06 mmol) dropwise. The reaction was stirred at 0° C. for 1 hour and then allowed to increase to room temperature and stirred for 23 h. The reaction mixture was then diluted with DCM (1 mL) and added directly to a silica column and purified by flash column chromatography (stationary phase; silica gel 230-400 mesh, mobile phase; 4:1, hexane/ethyl acetate) to yield 28.9 an off white solid (0.02 g, 76%).

¹H NMR (CDCl₃, 400 MHz) δ_{H} ppm: 1.53 (9H, s, C(CH₃)₃, 2.11 (1H, m, CH₂), 2.37 (1H, m, CH₂), 2.53 (1H, m, CH₂), 3.02 (1H, m, CH₂), 3.70 (3H, s, OMe), 3.90 (3H, s, OMe), 3.93 (3H, s, OMe), 3.94 (3H, s, OMe), 4.18 (1H, m, CHOH), 6.36 (1H, d, J=4.84 Hz, C═CH), 6.39 (1H, s, ArH), 6.79 (2H, s, 2×ArH), 7.09 (1H, s, ArH), 8.20 (1H, s, br, NH)

¹³C NMR (CDCl₃, 400 MHz) δ_{c} ppm: 21.39 (CH₂), 22.25 (C(CH₃)₃), 27.90 (C(CH₃)₃), 42.86 (CH₂), 55.31 (OMe), 55.60 (OMe), 60.42 (OMe), 61.09 (OMe), 69.42 (CHOH), 108.43 (ArCH), 109.00 (ArCH), 117.11 (ArCH), 122.00 (ArCH), 127.48 (QC), 127.65 (QC), 131.52 (CH═C), 133.70 (QC), 134.91 (QC), 138.39 (QC), 140.92 (QC), 147.73 (QC), 150.28 (QC), 150.63 (QC), 152.28 (QC)

vₘₐₓ (DCM)/cm⁻¹: 2066.14, 1643.67, 1528.58, 1488.47, 1406.30

HRMS: calculated 494.2155, found 494.2211, molecular formula (C₂₆H₃₃NNaO₇).

Melting Point: 180° C.

### Step 8

### Oxidation: Synthesis of tert-butyl (2-methoxy-5-(2,3,4-trimethoxy-7-oxo-6,7-dihydro-5H-benzo[7]annulen-9-yl)phenyl)carbamate 28.10

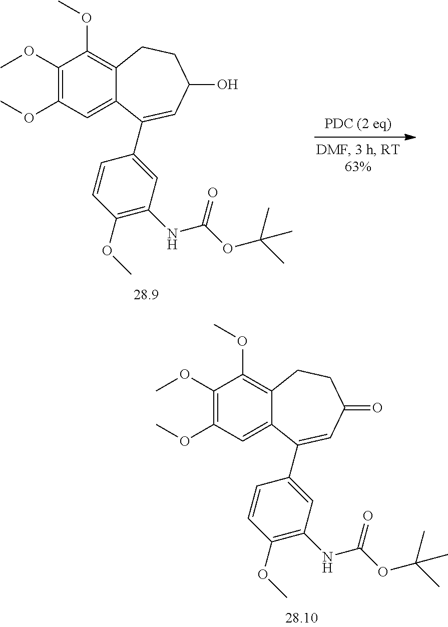

To a stirred solution of 22.9 (0.08 g, 0.17 mmol) in DMF (2 mL) was added pyridinium dichromate (0.13 g, 0.34 mmol). After 3 h the reaction was quenched by the slow addition of 2M HCl aqueous solution (15 mL) and the mixture was extracted with diethyl ether (3×25 mL). The organic fractions were dried over MgSO₄, filtered and concentrated in vacuo. The resulting residue was then purified by flash column chromatography (stationary phase; silica gel 230-400 mesh, mobile phase; 4:1, hexane/ethyl acetate) to yield 28.10 as a yellow oil (0.05 g, 63%).

¹H NMR (CDCl₃, 400 MHz) δ_{H} ppm: (1.51 (9H, s, C(CH₃)₃, 2.72 (2H, m, CH₂), 3.15 (2H, m, CH₂), 3.64 (3H, s, OMe), 3.91 (3H, s, OMe), 3.93 (3H, s, OMe), 3.96 (3H, s, OMe), 6.40 (1H, s, C═CH), 6.41 (1H, s, ArH), 6.85 (1H, d, J=8.5 Hz, ArH), 6.96 (1H, dd, J₁=2 Hz, J₂=8.5 Hz, ArH), 7.10 (1H, s, ArH), 8.11 (1H, s, br, NH)

¹³C NMR (CDCl₃, 400 MHz) δ_{c} ppm: 20.24 (CH₂), 28.33 (C(CH₃)₃), 45.50 (CH₂), 55.79 (OMe), 56.11 (OMe), 60.92 (OMe), 61.39 (OMe), 80.60 (C(CH₃)₃), 109.39 (ArCH), 112.18 (CH═C), 118.74 (ArCH), 123.56 (ArCH), 127.89 (QC), 128.42 (ArCH), 129.22 (QC), 132.55 (QC), 135.57 (QC), 143.25 (QC), 148.15 (QC), 149.93 (QC), 151.02 (QC), 152.12 (QC), 152.57 (QC), 204.01 (C═O)

vₘₐₓ (DCM)/cm⁻¹: 3435.99, 2930.00, 2854.43, 1728.64, 1652.10, 1590.88

HRMS: calculated 470.2179, found 470.2173, molecular formula (C₂₆H₃₂NO₇).

### Step 9

### Removal of BOC Group: Synthesis of 9-(3-amino-4-methoxyphenyl)-2,3,4-trimethoxy-5H-benzo[7]annulen-7(6H)-one 28.11

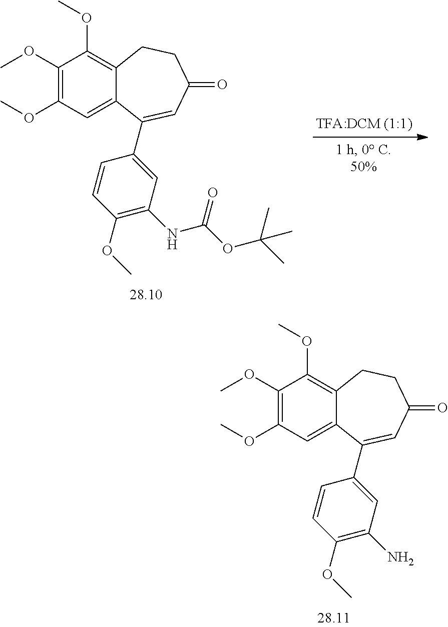

To a round bottom flask containing 28.10 (0.04 g, 0.11 mmol) under an atmosphere of nitrogen was added a mixture of TFA and dry DCM (2 mL, 1:1) at 0° C. After 1 hour the solvent was removed in vacuo. The resulting residue was then washed with sodium bicarbonate saturated aqueous solution (5 mL) and extracted with diethyl ether (3×5 mL). The organic fractions were dried over MgSO₄, filtered and concentrated in vacuo. The resulting residue was then purified by flash column chromatography (stationary phase; silica gel 230-400 mesh, mobile phase; 4:1, hexane/ethyl acetate) to yield 28.11, as a yellow oil (0.02 g, 50%).

¹H NMR (CDCl₃, 400 MHz) δ_{H} ppm: 2.72 (2H, m, CH₂), 3.15 (2H, m, CH₂), 3.65 (3H, s, OMe), 3.91 (3H, s, OMe), 3.92 (3H, s, OMe), 3.96 (3H, s, OMe), 6.39 (1H, s, C═CH), 6.43 (1H, s, ArH), 6.74 (2H, m, 2×ArH), 6.78 (1H, d, J=8.7 Hz, ArH)

¹³C NMR (CDCl₃, 400 MHz) δ_{c} ppm: 20.28 (CH₂), 45.71 (CH₂), 55.57 (OMe), 56.09 (OMe), 60.95 (OMe), 61.43 (OMe), 109.76 (ArCH), 112.01 (ArCH), 115.54 (ArCH), 119.78 ArCH), 127.82 (CH═C), 129.06 (QC), 132.71 (QC), 135.45 (QC), 135.80 (QC), 143.14 (QC), 148.07 (QC), 149.87 (QC), 151.00 (QC), 152.30 (QC), 204.32 (C═O)

vₘₐₓ (DCM)/cm⁻¹: 2933.52, 1651.25, 1513.03, 1492.79, 1464.70

HRMS: calculated 370.1654, found 370.1657, molecular formula (C₂₇H₃₈O₆Si).

### Step 10

### Salt Formation Synthesis of 2-methoxy-5-(2,3,4-trimethoxy-7-oxo-6,7-dihydro-5H-benzo[7]annulen-9-yl)benzenaminium chloride 28

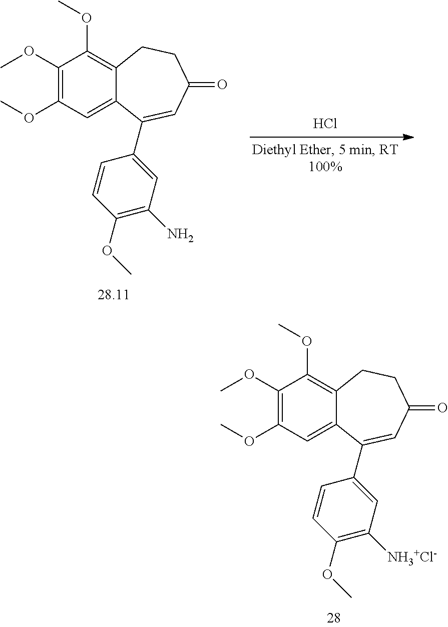

Gaseous HCl was bubbled through a solution of 28.11 (0.02 g, 0.05 mmol) in diethyl ether (2 mL). The resulting brown precipitate was allowed to settle and the diethyl ether supernatant was removed. The brown precipitate was then washed with diethyl ether (3×2 mL) and then dried under vacuum to yield 28 as a brown solid (0.02 g, 100%).

### Synthesis of 2-methoxy-5-(4, 4,5,5-tetramethyl-1,3,2-dioxaborolan-2-yl)aniline 28.12

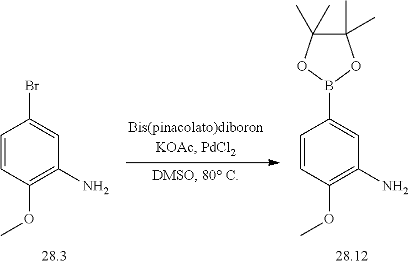

To potassium acetate (8.9 g, 90.83 mmol), bis-(pinacolato)-diboron (8.1 g, 31.86 mmol) and bis(diphenylphosphine) ferrocene dichloropalladium (II) (0.34 g, 0.48 mmol) was added an anhydrous solution of 5-bromo-2-methoxyaniline (3.22 g, 15.93 mmol) in DMSO (40 mL) under anhydrous conditions in an atmosphere of nitrogen. The mixture was stirred at 80° C. for 8 h after which time the reaction was quenched with saturated NaCl aqueous solution (30 mL) and extracted with diethyl ether (3×20 mL). The combined ether extracts were dried over magnesium sulphate, filtered and concentrated. The crude product was purified by flash column chromatography (stationary phase; silica gel 230-400 mesh, mobile phase; 9:1 hexane/ethyl acetate). All homogenous fractions were collected and reduced in volume to afford the product 28.12 as a brown syrup (3.18 g, 80%).

νₘₐₓ (DCM)/cm⁻¹: 2926.38, 1599.02, 1431.11, 1356.01, 1221.39, 1142.48

¹H NMR (CDCl₃, 400 MHz) δ_{H} ppm: 1.35 (12H, s, 2×C(CH₃)₂), 3.89 (3H, s, OCH₃), 6.15 (2H, br, NH₂), 7.18 (1H, d, J 8.0 Hz, ArH), 7.25 (1H, d, J 2.5 Hz, ArH), 7.29 (1H, s, ArH).

¹³C NMR δ_{c} ppm: 24.5 (4×CH₃), 55.1 (OCH₃), 83.2 (2×C(CH₃)₂) 105.5 (ArCH), 113.7 (ArC), 120.6 (ArCH), 125.8 (ArCH), 135.0 (ArC), 149.7 (ArC).

### Suzuki Coupling (Free Aniline) to yield 5-(7-((tert-butyldiphenylsilyl)oxy)-2,3,4-trimethoxy-6,7-dihydro-5H-benzo[7]annulen-9-yl)-2-methoxyaniline 28.13

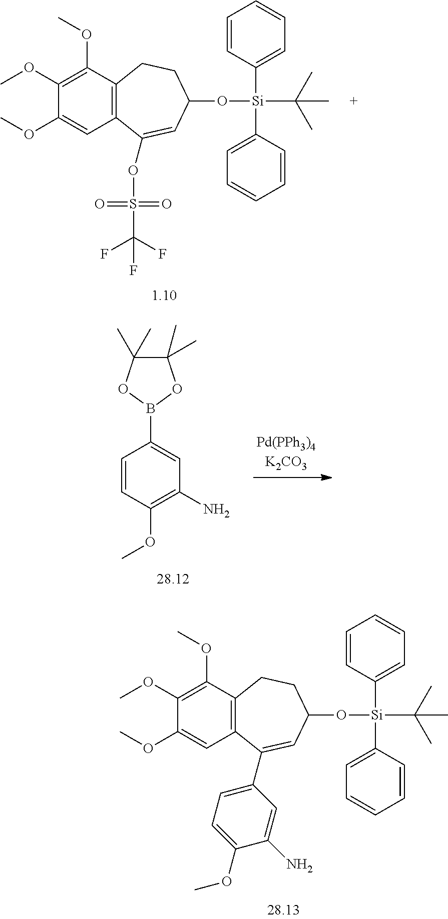

To a flask containing 7-((tert-butyldiphenylsilyl)oxy)-2,3,4-trimethoxy-6,7-dihydro-5H-benzo[7]annulen-9-yl trifluoromethanesulfonate 1.10 (2.25 g, 3.54 mmol) was added 2-methoxy-5-(4, 4,5,5-tetramethyl-1,3,2-dioxaborolan-2-yl)aniline 28.12 (970 mg, 3.89 mmol), K₂CO₃ (1.32 g, 9.56 mmol), and tetrakis-(triphenylphosphine)-palladium (0) (30 mg, 0.18 mmol). The mixture was dissolved in a mixture of benzene, ethanol and water (3:1:1, 10 mL). The resulting mixture was heated to 70° C. for thirty min. The reaction was quenched by the addition of water (1×20 mL) and the product was extracted with diethyl ether (3×30 mL). The organic fractions were combined, dried over MgSO₄, filtered and concentrated in vacuo. The resulting residue was purified by flash column chromatography (stationary phase; silica gel 230-400 mesh, mobile phase; 6:1, hexane/ethyl acetate). All homogenous fractions were collected and the solvent was evaporated to afford 5-(7-((tert-butyldiphenylsilyl)oxy)-2,3,4-trimethoxy-6,7-dihydro-5H-benzo[7]annulen-9-yl)-2-methoxyaniline as a yellow oil 28.13 (2.16 g, 100%).

νₘₐₓ (DCM)/cm⁻¹: 2931.09, 1488.27, 1112.69, 737.95, 702.93

¹H NMR (CDCl₃, 400 MHz) δ_{H} ppm: 1.11 (9H, s, C(CH₃)₃), 2.08-2.37 (4H, m, 2×CH₂), 3.68 (3H, s, OCH₃{C-ring}), 3.74 (3H, s, OCH₃), 3.89 (3H, s, OCH₃), 3.90 (3H, s, OCH₃), 4.18 (1H, m, CHOSi), 6.15 (2H, br, NH₂), 6.22 (1H, d, J=5.0 Hz, C═CH), 6.26 (1H, s, ArH (A-ring)), 6.42 (1H, s, ArH H (C-ring)), 6.50 (1H, d, ArH (C-ring)), 6.72 (1H, d, ArH (C-ring)), 7.28-7.46 (6H, m, ArH (diphenyl silyl)), 7.62-7.72 (4H, m, ArH (diphenyl silyl).

¹³C NMR (CDCl₃, 400 MHz) δ_{c} ppm: 18.68 (C(CH₃)), 21.39 (CH₂), 26.6 (3×C(CH₃)₃), 43.37 (CH₂), 55.15 (OCH₃), 55.57 (OCH₃), 60.44 (OCH₃), 61.10 (OCH₃), 70.89 (CHOSi), 108.2 (ArCH), 109.06 (ArCH), 114.1 (ArCH), 118.1 (ArCH), 127.00 (2×ArCH), 127.05 (2×ArCH), 127.48 (ArC), 129.01 (2×ArCH), 131.97 (ArCH), 133.91 (ArC), 133.96 (ArC), 134.05 (ArC), 135.18 (ArC), 135.31 (2×ArCH), 135.45 (2×ArCH), 137.45 (ArC), 146.47 (ArC), 150.11 (ArC), 150.40 (ArC).

Removal of silyl protecting group to give 9-(3-amino-4-methoxyphenyl)-2,3,4-trimethoxy-6,7-dihydro-5H-benzo[7]annulen-7-ol28.14

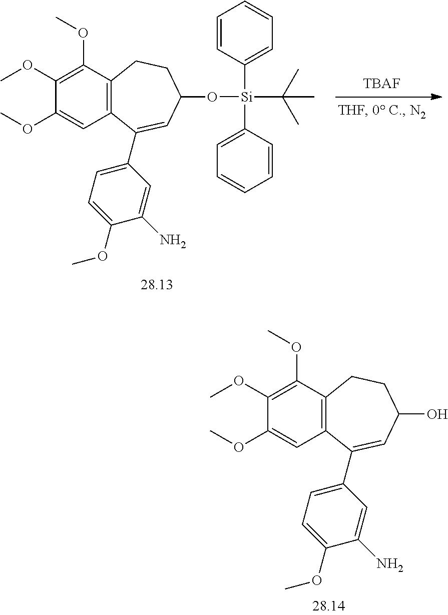

To a stirred solution of 5-(7-((tert-butyldiphenylsilyl)oxy)-2,3,4-trimethoxy-6,7-dihydro-5H-benzo[7]annulen-9-yl)-2-methoxyaniline 28.13 (2.6 g, 4.2 mmol) in THF (15 mL) was added 1 M TBAF (4.2 mL, 4.2 mmol) in THF at 0° C. The reaction was brought to room temperature. After 12 h the reaction mixture was applied directly to a flash column. The product was purified by flash column chromatography (stationary phase; silica gel 230-400 mesh, mobile phase; 2:1, hexane/ethyl acetate). All homogenous fractions were collected and the solvent was evaporated to afford 9-(3-amino-4-methoxyphenyl)-2,3,4-trimethoxy-6,7-dihydro-5H-benzo[7]annulen-7-ol as a white solid 28.14 (1.43 g, 92%).

νₘₐₓ (DCM)/cm⁻¹: 2960.67, 2933.63, 2855.96, 1593.66, 1487.76, 1235.81, 1112.45, 704.20

¹H NMR (CDCl₃, 400 MHz) δ_{H} ppm: 2.12 (1H, m, CH₂), 2.35 (1H, m, CH₂), 2.52 (1H, m, CHH₂), 3.03 (1H, m, CH₂), 3.70 (3H, s, OCHH₃), 3.88 (3H, s, OCH₃), 3.91 (3H, s, OCHH₃), 3.93 (3H, s, OCH₃), 4.14 (1H, m, CHOH), 5.32 (1H, s, br, OHH), 6.25 (1H, d, J=5.0 Hz, C═CH), 6.18 (2H, d, NH₂), 6.40 (1H, s, ArH (A-ring)), 6.70 (1H, d, J=2.0 Hz, 8.5 Hz, ArH (C-ring)), 6.75 (1H, d, J=2.0 Hz, ArH (C-ring)), 6.88 (1H, m, ArH (C-ring)).

¹³C NMR (CDCl₃, 400 MHz) δ_{c} ppm: 21.6 (H₂), 43.2 (CH₂), 55.1 (OCH₃), 55.58 (OCH₃), 60.44 (OCH₃), 61.10 (OCH₃), 69.7 (CHOH), 108.34 (ArCH), 109.58 (ArCH), 114.26 (ArCH), 117.91 (ArCH), 127.51 (ArC), 130.74 (ArCH), 133.65 (ArC), 135.04 (ArC), 135.29 (ArC), 138.54 (ArC), 140.87 (ArC), 146.59 (ArC), 150.23 (ArC), 150.63 (ArC).

### Selective Fmoc Protection of the Aniline to Give (9H-fluoren-9-yl)methyl (5-(7-hydroxy-2, 3,4-trimethoxy-6,7-dihydro-5H-benzo[7]annulen-9-yl)-2-methoxyphenyl)carbamate 28.15

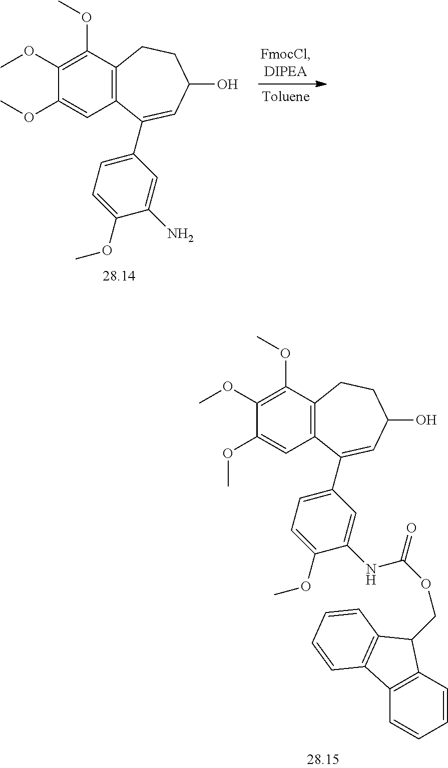 [SMILES: COc1ccc(C2=CC(O)CCc3c2cc(OC)c(OC)c3OC)cc1N ; "FmocCl,DIPEA" ; COc1ccc(C2=CC(O)CCc3c2cc(OC)c(OC)c3OC)cc1NC(=O)OCC1c2ccccc2-c2ccccc21]

To a solution of the aniline 28.14 (5.06 g, 13.62 mmol) and DIPEA (4.76 mL, 28.5 mmol) in toluene (25 mL) was added Fmoc chloride (7.05 g, 27.24 mmol) in toluene (20 mL). After 3 h at room temperature the solvent was removed under vacuum and the product was isolated by flash column chromatography (stationary phase; silica gel 230-400 mesh, mobile phase; 2:1, hexane/ethyl acetate). All homogenous fractions were collected and the solvent was evaporated to afford (9H-fluoren-9-yl)methyl (5-(7-hydroxy-2,3,4-trimethoxy-6,7-dihydro-5H-benzo[7]annulen-9-yl)-2-methoxyphenyl)carbamate 28.15 as a white solid (8.09 g, 100%).

νₘₐₓ (DCM)/cm⁻¹: 1264.48, 732.70, 702.92

¹H NMR (CDCl₃, 400 MHz) δ_{H} ppm: 2.14 (1H, m, CH₂), 2.39 (1H, m, CH₂), 2.54 (1H, m, CH₂), 3.05 (1H, dd, CH₂), 3.70 (3H, s, OCH₃), 3.92 (3H, s, OCH₃), 3.94 (6H, s, 2×OCH₃), 4.19 (1H, m, CHOH), 4.33 (1H, t, CH (Fmoc)), 4.51 (2H, q, CH₂ (Fmoc)), 5.66 (1H, s, br, OH), 6.36 (1H, d, J=5.0 Hz, C═CH), 6.40 (1H, s, ArH (A-ring)), 6.83 (1H, d, J=2.0 Hz, 8.5 Hz, ArH (C-ring)), 6.90 (1H, d, J=2.0 Hz, ArH (C-ring)), 7.29 (1H, d, J=8.0 Hz, ArH (C-ring)), 7.36 (2H, t, ArH (Fmoc)), 7.45 (2H, t, ArH (Fmoc)), 7.66 (2H, d, ArH (Fmoc)), 7.81 (2H, d, ArH (Fmoc)), 8.22 (2H, br, NH₂),

¹³C NMR (CDCl₃, 400 MHz) δ_{c} ppm: 21.9 (CH₂), 43.5 (CH₂), 47.2 (CH (Fmoc)), 55.86 (OCH₃), 56.07 (OCH₃), 60.89 (OCH₃), 61.57 (OCH₃), 67.07 (CH₂ (Fmoc)), 69.89 (CHOH), 108.86 (ArCH), 109.67 (ArCH), 120.1 (2×ArCH), 123.11 (ArCH), 125.08 (2×ArCH (Fmoc)), 127.13 (2×ArCH (Fmoc)), 127.3 (ArC), 127.8 (2×ArCH (Fmoc)), 128.19 (2×ArC Fmoc)), 132.21 (2×ArCH (Fmoc)), 134.3 (ArC), 135.22 (ArC), 138.78 (ArC), 141.34 (2×ArC (Fmoc)), 141.47 (ArC), 143.78 (ArC), 143.83 (ArC), 150.79 (ArC), 151.14 (ArC), 153.41 (C═O).

### Synthesis of 5,6,8,9-tetrahydro-9-(3-hydroxy-4-methoxyphenyl)-1,2,3-trimethoxybenzo[7]annulen-7-oximino 29

### Step 1

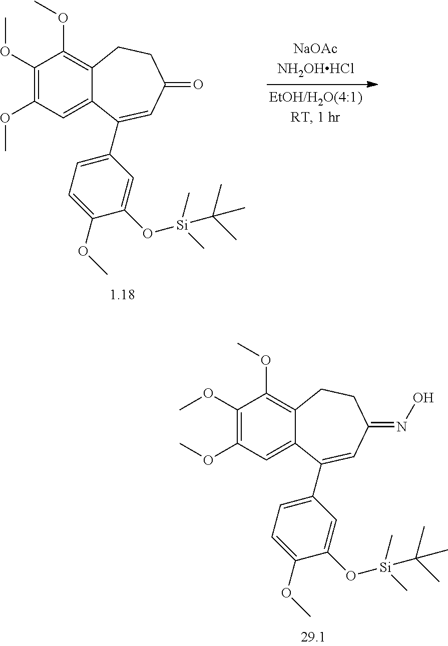 [SMILES: COc1ccc(C2=CC(=O)CCc3c2cc(OC)c(OC)c3OC)cc1O[Si](C)(C)C(C)(C)C ; COc1ccc(C2=C/C(=N/O)CCc3c2cc(OC)c(OC)c3OC)cc1O[Si](C)(C)C(C)(C)C ; "NaOAcNH2OH·HCl"]

To a stirred solution of 1.18 (0.20 g, 0.42 mmol, 1 Eq) in EtOH (4 mL) and H₂O (1 mL), was added hydroxylamine hydrochloride (0.04 g, 0.46 mmol, 1.1 Eq) and sodium acetate (0.06 g, 0.66 mmol, 1.6 Eq). The resultant mixture was stirred at room temperature for 1 hr, until complete consumption of the starting material was observed using TLC (Hexane/Ethyl Acetate 3:1). The mixture was diluted in water (10 mL) and the product was extracted using ether (3×15 mL). The product, 29.1, was isolated as a mixture of isomers by column chromatography using hexane/ethyl acetate (6:1) as the mobile phase, in the form of a yellow oil.

Yield: 0.16 g, 0.32 mmol, 76%.

¹H NMR (CDCl₃, 400 MHz) δ_{H} ppm: 0.16 (s, 6H, 2×CH₃), 0.99 (s, 9H, ^{t}Bu), 2.95-3.03 (m, 4H, 2×CH₂), 3.61 (s, 3H, OCH₃), 3.87 (s, 3H, OCH₃), 3.92 (s, 6H, 2×OCH₃), 6.31 (s, 1H), 6.55 (s, 1H), 6.83 (m, 2H), 6.95 (m, 1H).

¹³C NMR (CDCl₃, 100.71 MHz) δ_{c} ppm: −5.0 (CH₃), 13.7 (Q), 20.8 (CH₂), 25.3 (CH₃), 29.3 (CH₂), 55.0 (CH₃), 55.4 (CH₃), 60.5 (CH₃), 61.0 (CH₃), 110.0 (CH), 111.0 (CH), 121.2 (CH), 122.0 (CH), 123.7 (CH), 128.2 (Q), 133.1 (Q), 136.2 (Q), 141.7 (Q), 144.0 (Q), 144.7 (Q), 149.6 (Q), 150.4 (Q), 150.4 (Q).

MS (+ESI): Calculated Mass 499.2390 Found 500.1702 (M+H⁺).

### Step 2 Deprotection

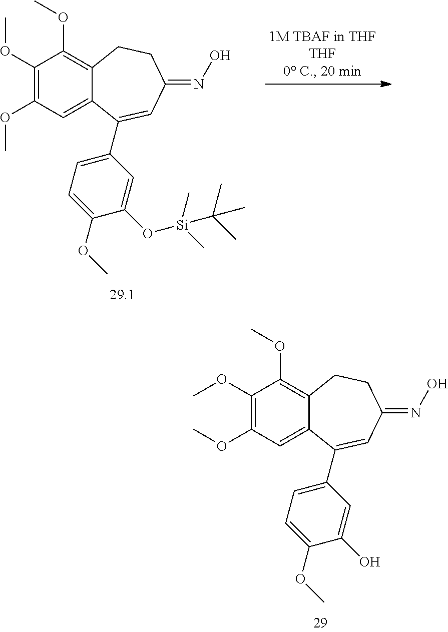 [SMILES: COc1ccc(C2=C/C(=N/O)CCc3c2cc(OC)c(OC)c3OC)cc1O[Si](C)(C)C(C)(C)C ; "1M TBAF in THFTHF0° C., 20 min" ; COc1ccc(C2=C/C(=N/O)CCc3c2cc(OC)c(OC)c3OC)cc1O]

To a stirred solution of 29.1 (0.02 g, 0.04 mmol, 1 Eq) in THF (1 mL) was added 1M TBAF (0.04 mL, 0.04 mmol, 1 Eq). The mixture was stirred for 20 min. TLC (Hexane/Ethyl Acetate 1:1) showed complete consumption of starting materials. The solvents were removed in vacuo and the remaining residue was placed directly on a column. The product was isolated as a brown solid using a mobile phase system of hexane/ethyl acetate (2:1).

Yield: 0.01 g, 0.39 mmol, 97%

¹H NMR (CDCl₃, 400 MHz) δ_{H} ppm: 2.93-3.02 (m, 4H, 2×CH₃), 3.62 (s, 3H, OCH₃), 3.91 (s, 3H, OCH₃), 3.93 (s, 3H, OCH₃), 3.96 (s, 3H, OCH₃), 6.34 (s, 1H), 6.57 (s, 1H), 6.84 (d, J=8.35 Hz, 1H, ArH), 6.89 (d, J=8.92 Hz, 1H, ArH), 6.95 (1H, s, ArH)

¹³C NMR (CDCl₃, 100.71 MHz) δ_{c} ppm: 21.3 (CH₂), 29.8 (CH₂), 56.0 (2×CH₃), 60.9 (CH₃), 61.5 (CH₃), 110.2 (CH), 110.6 (CH), 115.3 (CH), 120.8 (CH), 124.4 (CH), 128.8 (Q), 133.5 (Q), 137.3 (Q), 142.3 (Q), 145.0 (Q), 146.5 (Q), 150.1 (Q), 150.9 (Q).

MS (+ESI): Calculated Mass 385.1525 Found 386.1598 (M+H⁺).

### Synthesis of 2-[9-(3-Amino-4-methoxy-phenyl)-2,3,4-trimethoxy-5,6-dihydro-benzocyclohepten-7-ylideneaminooxy]-N-hydroxy-acetamide 30

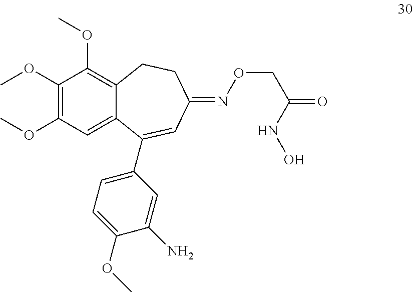 [SMILES: COc1ccc(C2=C/C(=N/OCC(=O)NO)CCc3c2cc(OC)c(OC)c3OC)cc1N]

### Synthesis of (E)-2-(((9-(3-((tert-butoxycarbonyl)amino)-4-methoxyphenyl)-2,3,4-trimethoxy-5H-benzo[7]annulen-7(6H)-ylidene)amino)oxy)acetic acid 30.1.

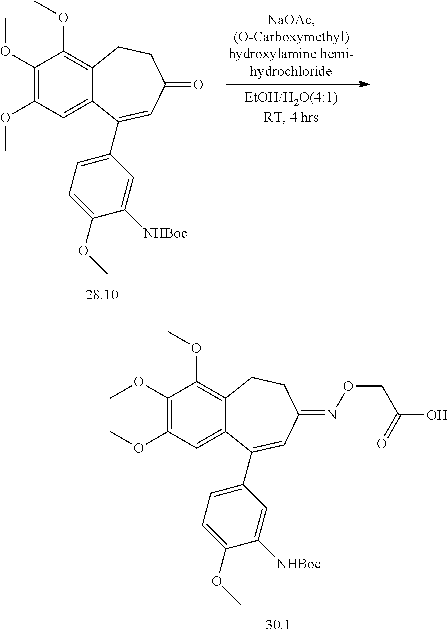 [SMILES: COc1ccc(C2=CC(=O)CCc3c2cc(OC)c(OC)c3OC)cc1NC(=O)OC(C)(C)C ; "NaOAc,(O-Carboxymethyl)hydroxylamine hemi-hydrochloride" ; COc1ccc(C2=C/C(=N/OCC(=O)O)CCc3c2cc(OC)c(OC)c3OC)cc1NC(=O)OC(C)(C)C]

To a stirred solution of tert-butyl (2-methoxy-5-(2,3,4-trimethoxy-7-oxo-6,7-dihydro-5H-benzo[7]annulen-9-yl)phenyl)carbamate 28.10 (0.0381 g, 0.081 mmol) in EtOH (2.5 mL) and H₂O (0.5 mL), was added (O-carboxymethyl)hydroxylamine hemi-hydrochloride (0.0115 g, 0.105 mmol) and NaOAc (0.0107 g, 0.129 mmol). The resultant mixture was stirred at room temperature for 4 hrs, until complete consumption of the starting material was observed using TLC (hexane/ethyl acetate 1:1). The mixture was diluted in water (10 mL) and the product was extracted using ether (3×15 mL). The product 30.1 was isolated as a mixture of isomers by column chromatography using ethyl acetate as the mobile phase, in the form of a yellow oil.

Yield: 0.0307 g, 0.056 mmol, 70%.

¹H NMR (CDCl₃, 400 MHz) δ_{H} ppm: 1.51 (s, 9H, ^{t}Bu), 2.75-2.79 (m, 1H, CH₂ minor isomer), 2.90-2.97 (m, 1H, CH₂ major isomer), 2.97-3.02 (m, 1H, CH₂ major isomer), 3.02-3.06 (m, 1H, CH₂ minor isomer), 3.63 (s, 3H, OCH₃), 3.89 (s, 1H, OCH₃ minor isomer), 3.90 (s, 2H, OCH₃ major isomer), 3.91 (s, 2H, OCH₃ major isomer), 3.92 (s, 1H, OCH₃ minor isomer), 3.92 (s, 2H, OCH₃ major isomer), 3.94 (s, 1H, OCH₃ minor isomer), 4.67 (s, 1H, CH₂ minor isomer), 4.69 (s, 1H, CH₂ major isomer), 6.34 (s, 1H, ArH major isomer), 6.38 (s, 1H, ArH minor isomer), 6.55 (s, 1H), 6.80-6.86 (m, 1H, ArH), 6.95-7.03 (m, 2H), 7.12 (s, 1H, NHBoc).

¹³C NMR (CDCl₃, 100.71 MHz) δ_{c} ppm: 14.2 (Q), 21.3 (CH₂ major isomer), 21.9 (CH₂ minor isomer), 28.4 (CH₃), 29.7 (CH₂ minor isomer), 30.3 (CH₃ minor isomer), 32.9 (CH₂ major isomer), 55.7 (CH₃), 56.1 (CH₃), 60.8 (CH₃), 61.3 (CH₃), 70.1 (CH₂ minor isomer), 70.3 (CH₂ major isomer), 109.4 (CH), 110.9 (CH major isomer), 111.9 (CH minor isomer), 123.3 (CH), 123.5 (CH), 123.6 (CH), 127.7 (Q), 128.2 (Q), 128.5 (Q), 130.0 (Q), 133.4 (Q), 136.6 (Q), 142.3 (Q), 146.4 (Q), 147.5 (Q), 150.0 (Q), 162.0 (Q, C═N), 174.0 (Q, COOH).

MS (+ESI): Calculated Mass 542.2264. Found 543.2333 (M+H)⁺.

### Synthesis of the pentafluorophenol ester of (E)-2-(((9-(3-((tert-butoxycarbonyl)amino)-4-methoxyphenyl)-2,3,4-trimethoxy-5H-benzo[7]annulen-7(6H)-ylidene)amino)oxy)acetic acid

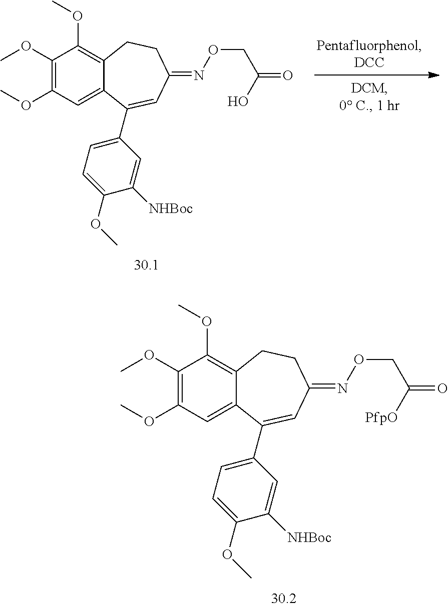

To a stirred solution of (E)-2-(((9-(3-((tert-butoxycarbonyl)amino)-4-methoxyphenyl)-2,3,4-trimethoxy-5H-benzo[7]annulen-7(6H)-ylidene)amino)oxy)acetic acid 30.1 (0.0312 g, 0.058 mmol) in DCM (1 mL) under an atmosphere of N₂ at 0° C. was added a solution of pentafluorophenol (0.0116 g, 0.063 mmol) in DCM (0.75 mL). This was followed by the subsequent addition of DCC (0.0137 g, 0.063 mmol) in DCM (0.75 mL). The solution was stirred for 1 hr, after which time the dicyclohexylurea by-product was removed via filtration.

The crude DCM residue was then concentrated in vacuo to be purified using column chromatography with hexane/ethyl acetate (7:1) as the mobile phase yielding the desired compound 30.2 as a colourless oil, in a mixture of syn- and anti-isomers.

Yield: 0.0029 g, 0.0412 mmol, 71%.

¹H NMR (CDCl₃, 600 MHz) δ_{H} ppm: 1.51 (s, 3H, ^{t}Bu minor isomer), 1.52 (s, 3H, ^{t}Bu major isomer), 2.76-2.80 (m, 1H, ring CH₂ minor isomer), 2.97 (br s, 1H, ring CH₂ major isomer), 3.00-3.05 (m, 2H, ring CH₂, mixture of isomers), 3.62 (s, 1H, OCH₃ minor isomer), 3.63 (s, 2H, OCH₃ major isomer), 3.90 (s, 1H, OCH₃ minor isomer), 3.93 (s, 4H, 2×OCH₃ major isomers), 3.94 s, 1H, OCH₃ minor isomer), 4.97 (s, 1H, CH₂ minor isomer), 5.01 (s, 1H, CH₂ major isomer), 6.35 (s, 1H major isomer), 6.38 (s, 1H, minor isomer), 6.56 (s, 1H), 6.82-6.86 (m, 1H), 6.94-6.97 (m, 1H major isomer), 6.99-7.02 (m, 1H minor isomer), 7.05 (s, 1H, NHBoc), 7.08 (s, 1H, ArH minor isomer), 7.09 (s, 1H, ArH major isomer).

¹³C NMR (CDCl₃, 151.71 MHz) δ_{c} ppm: 21.2 (CH₂ major isomer), 21.9 (CH₂ minor isomer), 24.8 (Q), 25.4 (Q), 28.3 (CH₃ minor isomer), 28.3 (CH₃ major isomer), 32.8 (CH₂ major isomer), 36.3 (CH₂ minor isomer), 55.8 (CH₃), 56.1 (CH₃), 60.9 (CH₃), 61.4 (CH₃), 69.7 (CH₂ minor isomer), 69.9 (CH₂ major isomer), 109.4 (CH), 111.0 (CH major isomer), 112.0 (CH minor isomer), 117.8 (CH), 123.3 (CH), 123.6 (CH), 127.6 (Q), 127.7 (Q), 129.3 (Q), 133.4 (Q), 136.6 (Q), 137.0 (Q), 138.7 (Q), 142.3 (Q), 142.6 (Q), 146.5 (Q), 147.1 (Q), 147.5 (Q), 147.7 (Q), 149.9 (Q), 150.0 (Q), 150.9 (Q), 152.8 (Q), 158.7 (Q), 162.4 (Q, C═N), 166.0 (Q, C═O major isomer), 166.3 (Q, C═O minor isomer).

MS (−ESI): Calculated Mass 723.2287. Found 709.2244 (M+H)⁺.

### Synthesis of (E)-tert-butyl (5-(7-((2-(hydroxyamino)-2-oxoethoxy)imino)-2,3,4-trimethoxy-6,7-dihydro-5H-benzo[7]annulen-9-yl)-2-methoxyphenyl)carbamate 30.3

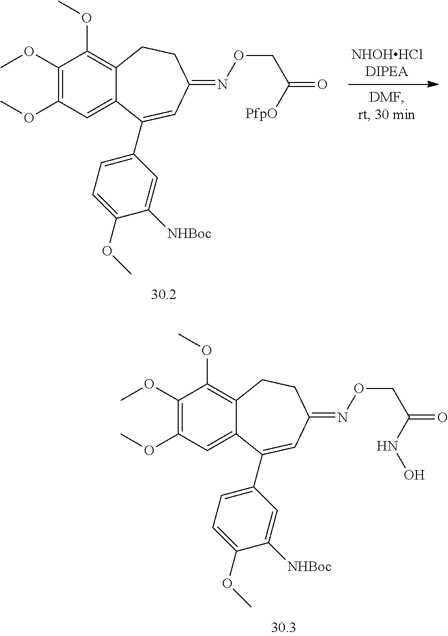

A solution of the pentafluorophenol ester 30.2 (0.0292 g, 0.0412 mmol), hydroxylamine hydrochloride (0.0029 g, 0.0412 mmol) and DIPEA (0.0071 mL, 0.0412 mmol) in DMF (1 mL) under N₂ was stirred at room temperature for 30 min. At this time, the produced was extracted by washing between 0.5 M HCl (15 mL) and ether (3×15 mL). The combined ether extracts were washed with water (15 mL) and dried over MgSO₄. Following concentration in vacuo, the product 30.3 was purified using column chromatography with ethyl acetate as the mobile phase as a yellow oil in a mixture of syn and anti isomers.

Yield: 0.0194 g (0.0346 mmol, 84.4%).

¹H NMR (CDCl₃, 400 MHz) δ_{H} ppm: 1.51 (s, 6H, ^{t}Bu major isomer), 1.53 (s, 3H ^{t}Bu minor isomer), 2.74-2.76 (m, 1H, ring CH₂ minor isomer), 2.98-3.04 (m, 3H, ring CH₂+ring CH₂ major isomer), 3.62 (s, 2H, OCH₃ major isomer), 3.64 (s, 1H, OCH₃ minor isomer), 3.89 (s, 1H, OCH₃ minor isomer), 3.91 (s, 2H, OCH₃ major isomer), 3.92 (s, 2H, OCH₃ major isomer), 3.92 (s, 1H, OCH₃ minor isomer), 3.93 (s, 2H, OCH₃ major isomer), 3.94 (s, 1H, OCH₃ minor isomer), 4.68 (br s, 2H, CH₂), 6.35 (s, 1H, ArCH major isomer), 6.39 (s, 1H, ArCH minor isomer), 6.51 (s, 1H, Alkene CH), 6.82 (m, 1H, ArCH), 6.91-6.94 (m, 1H, ArCH), 7.10 (s, 1H, ArCH major isomer), 7.13 (s, 1H, ArCH minor isomer), 8.13 (s, 1H, NHOH).

¹³C NMR (CDCl₃, 151.71 MHz) δ_{c} ppm: 21.1 (CH₂ major isomer), 21.8 (CH₂ minor isomer), 22.6 (Q, Boc), 28.3 (CH₃, Boc major isomer), 29.3 (CH₃, Boc minor isomer), 29.6 (CH₂ major isomer), 33.0 (CH₂ minor isomer), 55.7 (CH₃), 56.0 (CH₃), 60.8 (CH₃), 61.3 (CH₃), 71.3 (CH₂), 109.4 (CH), 110.8 (CH major isomer), 111.8 (CH minor isomer), 117.4 (CH), 118.5 (CH), 118.7 (CH), 123.1 (CH), 123.3 (CH), 123.4 (CH), 127.0 (Q), 128.3 (Q) 129.0 ( ) 133.0 (Q minor isomer), 133.2 (Q major isomer), 136.3 (Q major isomer), 136.5 (Q minor isomer), 142.4 (Q major isomer), 142.7 (Q minor isomer), 147.0 (Q), 147.5 (Q major isomer), 147.7 (Q major isomer), 150.0 (Q minor isomer), 150.0 (Q major isomer), 150.8 (Q minor isomer), 160.0 (Q major isomer), 152.7 (Q, C═N), 167.3 (Q, C═O).

MS (−ESI): Calculated Mass 557.2373. Found 556.2340 (M−H)⁻.

### Synthesis of (E)-2-a(9-(3-amino-4-methoxyphenyl)-2,3,4-trimethoxy-5H-benzo[7]annulen-7(6H)-ylidene)amino)oxy)-N-hydroxyacetamide 30

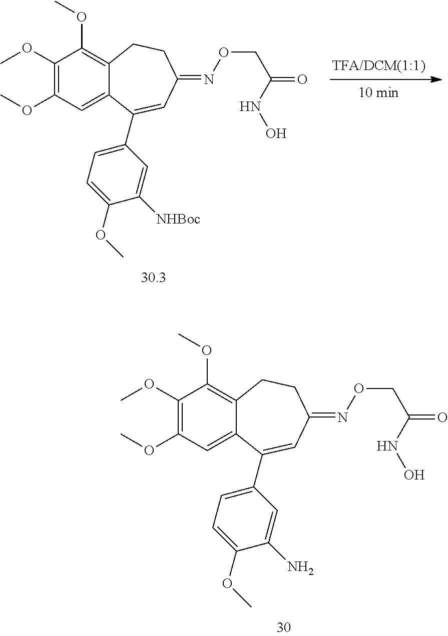 [SMILES: COc1ccc(C2=C/C(=N/OCC(=O)NO)CCc3c2cc(OC)c(OC)c3OC)cc1NC(=O)OC(C)(C)C ; "TFA/DCM(1:1)" ; COc1ccc(C2=C/C(=N/OCC(=O)NO)CCc3c2cc(OC)c(OC)c3OC)cc1N]

Under an atmosphere of N₂, tert-butyl (5-(7-((2-(hydroxyamino)-2-oxoethoxy)imino)-2,3,4-trimethoxy-6,7-dihydro-5H-benzo[7]annulen-9-yl)-2-methoxyphenyl)carbamate (0.0194 g, 0.0346 mmol), was dissolved in DCM (0.4 mL) and stirred for 2 min at 0° C. Trifluoroacetic acid (0.4 mL) was added dropwise to the flask. The mixture was stirred for 15 min, before the removal of the volatile constituents using a stream of N₂ gas. The product was isolated following an extraction between 5% NaHCO₃ (2 mL) and ether (3×2 mL). The ether layers were combined and washed with H₂O before being dried over MgSO₄. Removal of the solvent yielded the product 30 as a yellow solid.

¹H NMR (CDCl₃, 600 MHz) δ_{H} ppm: 2.91 (br m, 4H, 2×ring CH₂), 3.61 (br s, 3H, OCH₃), 3.87-3.96 (overlapping br s, 9H, 3×OCH₃), 4.86 (br s, 2H, CH₂), 6.29-6.41 (br m, alkene CH+ArH A ring), 6.45-6.58 (m, 2H, ArNH₂), 6.69-6.84 (br m, 3H, ArH).

MS (+ESI): Calculated Mass 457.1849. Found 458.1907 (M+H)⁺.

### Coupling tBOC Glutamate to the Aniline to Yield tert-butyl 1-(5-((Z)-6,7-dihydro-2,3,4-trimethoxy-7-oxo-5H-benzo[7]annulen-9-yl)-2-methoxyphenylcarbamoyl)-3-(tert-butoxycarbonyl)propylcarbamate 31.1

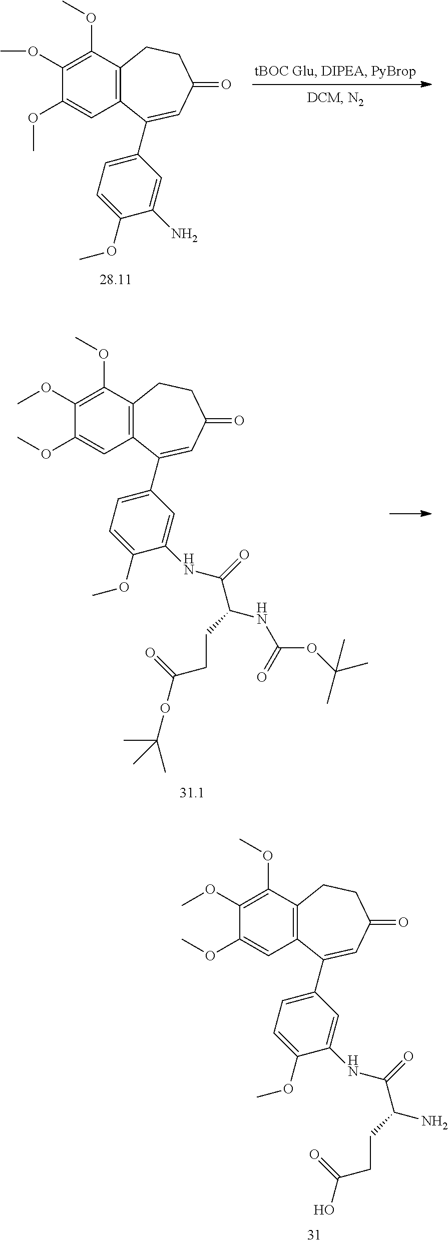

To a stirred solution of 9-(3-amino-4-methoxyphenyl)-2,3,4-trimethoxy-5H-benzo[7]annulen-7(6H)-one 28.11 (430 mg, 1.1 mmol) in dry DCM (15 mL) was added a solution of tBOC glutamate (1.03 g, 3.4 mmol), PyBrop (760 mg, 1.64 mmol) and DIPEA (0.74 mL, 4.36 mmol) in dry DCM (15 mL) at 0° C. The reaction temperature was allowed to increase to room temperature and left stirring for 6 h. The reaction was then quenched by the addition of 1M HCl (15 mL) and extracted into diethyl ether (3×15 mL). The organic fractions were combined, dried over MgSO₄, filtered and concentrated in vacuo. The product was isolated by flash column chromatography (stationary phase; silica gel 230-400 mesh, mobile phase; 3:1, hexane:ethyl acetate). All homogenous fractions were collected and the solvent was evaporated to afford tert-butyl 1-(5-((Z)-6,7-dihydro-2,3,4-trimethoxy-7-oxo-5H-benzo[7]annulen-9-yl)-2-methoxyphenylcarbamoyl)-3-(tert-butoxycarbonyl)propylcarbamate 31.1 as a white solid (720 mg, 100%).

νₘₐₓ (DCM)/cm⁻¹: 2936.48, 1492.44, 1262.94, 1116.23

¹H NMR (CDCl₃:CDOD₃, 1:1, 400 MHz) δ_{H} ppm: 1.44 (18H, s, 2×C(CH₃)₃), 1.946 (1H, m, CH₂(Glu)), 2.16 (1H, m, CH₂ (Glu)), 2.383 (2H, m, CH₂ (Glu)), 2.69 (2H, d, CH₂), 3.11 (2H, t, CH₂), 3.60 (3H, s, OCH₃), 3.88 (3H, s, OCH₃), 3.90 (3H, s, OCH₃), 3.93 (3H, s, OCH₃), 4.29 (1H, t, C═OCH (Glu)), 5.49 (1H, s, ArH (A-ring)), 6.35 (2H, d, C═CH and ArH (C-ring)), 6.85 (1H, d, 6.84 (1H, d, ArH (C-ring)), 7.03 (1H, d, ArH (C-ring)), 8.30 (1H, br, NH), 8.57 (CHNH (Glu)).

¹³C NMR (CDCl₃:CDOD₃, 1:1, 400 MHz) δ_{c} ppm: 19.7 (CH₂), 27.0 (CH₂ (Glu)), 31.4 (CH₂ (Glu)), 27.6 2×(C(CH₃)₃ (BOC)), 45.1 (CH₂), 54.55 (CHNH), 55.40 (OCH₃), 55.63 (OCH₃), 60.42 (OCH₃), 60.88 (OCH₃), 80.50 (2×C(CH₃)₃ (BOC)), 109.14 (ArCH), 111.61 (ArCH), 120.26 (ArCH), 124.66 (ArCH), 126.50 (ArC), 128.02 (ArCH), 128.68 (ArC), 131.91 (ArC), 134.99 (ArC), 142.84 (ArC), 148.46 (ArC), 149.49 (ArC), 150.62 (ArC), 151.47 (ArC), 169.45 (NHC═O (BOC)), 172.27 (NHC═O (Glu)), 172.28 (OC═O (Glu)) 203.66 (C═O).

### Removal of tBOC Protecting Group to Yield 2-amino-N-(5-((Z)-6,7-dihydro-2,3,4-trimethoxy-7-oxo-5H-benzo[7]annulen-9-yl)-2-methoxyphenyl)-4-methylpentanamide 31

HCl gas was bubbled through a solution of tert-butyl 1-(5-((Z)-6,7-dihydro-2,3,4-trimethoxy-7-oxo-5H-benzo[7]annulen-9-yl)-2-methoxyphenylcarbamoyl)-3-(tert-butoxycarbonyl)propylcarbamate 31.1 (720 mg) in methanol (10 mL). After 10 h, it could be seen by TLC that the reaction was complete. The solvent was removed under vacuum and the salt was washed with diethyl ether to give 31.

νₘₐₓ (DCM)/cm⁻¹: 2935.41, 1670.43, 1494.09, 1200.48

¹H NMR (CDCl₃:CDOD₃, 1:1, 400 MHz) δ_{H} ppm: 2.03 (2H, m, CH₂ (Glu)), 2.41 (2H, m, CH₂ (Glu)), 2.64 (2H, d, CH₂), 3.08 (2H, t, CH₂), 3.58 (3H, s, OCH₃), 3.81 (3H, s, OCH₃), 3.85 (3H, s, OCH₃), 3.91 (3H, s, OCH₃), 3.35 (1H, t, C═OCH (Glu)), 6.27 (1H, s, ArH (A-ring)), 6.38 (1H, s, C═CH), 6.91 (H, d, ArH (C-ring)), 7.10 (1H, d, 6.84 (1H, d, ArH (C-ring)), 7.53 (1H, d, ArH (C-ring)), 8.04 (1H, br, NH), 10.01 (COOH)

¹³C NMR (CDCl₃:CDOD₃, 1:1, 400 MHz) δ_{c} ppm: 19.4 (CH₂), 28.0 (CH₂ (Glu)), 333.0 (CH₂ (Glu)), 44.7 (CH₂), 54.55 (CHNH), 55.09 (OCH₃), 55.24 (OCH₃), 60.09 (OCH₃), 60.61 (OCH₃), 109.76 (ArCH), 111.63 (ArCH), 122.10 (ArCH), 125.66 (ArC), 126.00 (ArC), 127.17 (ArCH), 128.59 (ArCH), 131.68 (ArC), 134.15 (ArC), 142.85 (ArC), 149.36 (ArC), 150.40 (ArC), 150.59 (ArC), 152.14 (ArC), 168.77 (NHC═O (Glu)), 178.9 (OC═O (Glu)) 204.6 (C═O).

### Coupling tBOC Leucine to the Aniline to Yield tert-butyl 1-(5-((Z)-6,7-dihydro-2,3,4-trimethoxy-7-oxo-5H-benzo[7]annulen-9-yl)-2-methoxyphenylcarbamoyl)-3-methylbutylcarbamate 32.1

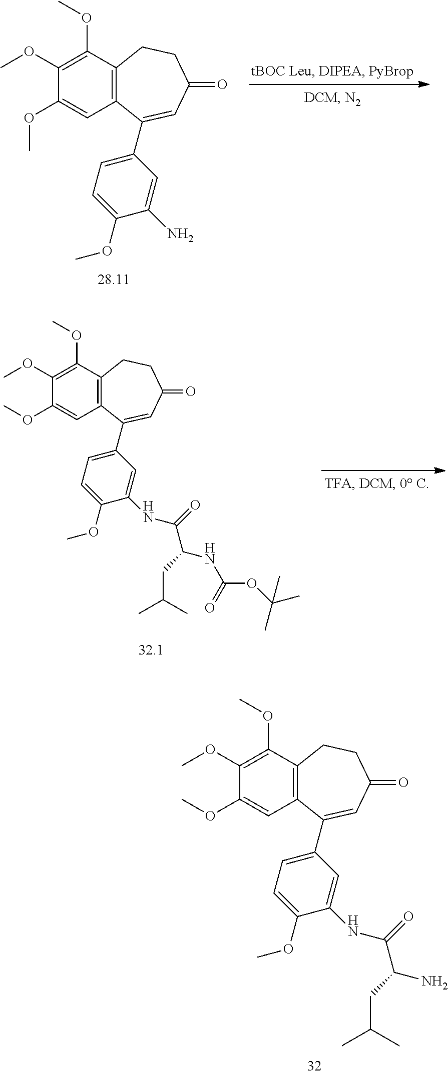

To a stirred solution of 9-(3-amino-4-methoxyphenyl)-2,3,4-trimethoxy-5H-benzo[7]annulen-7(6H)-one 28.11 (400 mg, 1.09 mmol) in dry DCM (15 mL) was added a solution of tBOC leucine (750 mg, 3.25 mmol), PyBrop (760 mg, 1.64 mmol) and DIPEA (0.56 mL, 4.36 mmol) in dry DCM (15 mL) at 0° C. The reaction temperature was allowed to increase to room temperature and left stirring for 6 h. The reaction was then quenched by the addition of 1M HCl (15 mL) and extracted into diethyl ether (3×15 mL). The organic fractions were combined, dried over MgSO₄, filtered and concentrated in vacuo. The product 32.1 was isolated by flash column chromatography (stationary phase; silica gel 230-400 mesh, mobile phase; 3:1, hexane/ethyl acetate). All homogenous fractions were collected and the solvent was evaporated to afford tert-butyl 1-(5-((Z)-6,7-dihydro-2,3,4-trimethoxy-7-oxo-5H-benzo[7]annulen-9-yl)-2-methoxyphenylcarbamoyl)-3-methylbutylcarbamate 32.1 as a white solid (622 mg, 98%).

νₘₐₓ (DCM)/cm⁻¹: 2957.55, 1708.56, 1167.16, 842.35

¹H NMR (CDCl₃, 400 MHz) δ_{H} ppm: 0.94 (6H, t, 2×CH₃ (Leu)), 1.45 (9H, s, C(CH₃)₃), 1.55 (1H, m, CH (Leu)), 1.72 (2H, t, CH₂ (Leu)), 2.68 (2H, d, CH₂), 3.10 (2H, t, CH₂), 3.58 (3H, s, OCH₃), 3.87 (3H, s, OCH₃), 3.89 (3H, s, OCH₃), 3.91 (3H, s, OCH₃), 4.28 (1H, t, C═OCH (Leu)), 5.16 (1H, s, ArH (A-ring)), 6.34 (1H, s, C═CH), 6.35 (1H, d, ArH (C-ring)), 6.84 (1H, d, ArH (C-ring)), 7.03 (1H, d, ArH (C-ring)), 8.32 (1H, br, NH), 8.51 (CHNH (Leu)).

¹³C NMR (CDCl₃, 400 MHz) δ_{c} ppm: 19.73 (CH₂), 21.42 (CH₃ (Leu)), 22.47 (CH₃ (Leu)), 24.36 (CH(CH₃)₂ (Leu)), 27.81 ((C(CH₃)₃ (BOC)), 40.54 (CH₂ (Leu)), 45.01 (CH₂), 53.46 (CHNH), 55.40 (OCH₃), 55.61 (OCH₃), 60.42 (OCH₃), 60.88 (OCH₃), 79.9 (C(CH₃)₃ (BOC)), 109.12 (ArCH), 111.62 (ArCH), 120.4 (ArCH), 125.0 (ArCH), 126.8 (ArC), 128.0 (ArCH), 128.7 (ArC), 131.9 (ArC), 135.2 (ArC), 143.1 (ArC), 148.5 (ArC), 149.4 (ArC), 150.6 (ArC), 151.7 (ArC), 155.4 (NHC═O (BOC)), 170.5 (NHC═O (Leu)), 203.5 (C═O).

### Removal of tBOC Protecting Group to yield 2-amino-N-(5-((Z)-6,7-dihydro-2,3,4-trimethoxy-7-oxo-5H-benzo[7]annulen-9-yl)-2-methoxyphenyl)-4-methylpentanamide 32

To a solution of tert-butyl 1-(5-((Z)-6,7-dihydro-2,3,4-trimethoxy-7-oxo-5H-benzo[7]annulen-9-yl)-2-methoxyphenylcarbamoyl)-3-methylbutylcarbamate 32.1 (622 mg) in dry DCM (3 mL) was added neat TFA (3 mL) drop-wise under an atmosphere of N₂ at 0° C. After one hour the reaction was quenched with 1M NaOH (10 mL) and the product was extracted into ethyl acetate (3×15 mL). The organic fractions were combined, dried over MgSO₄, filtered and concentrated in vacuo. The product was obtained in quantitative yield (515 mg, 100%). This was redissolved in methanol (5 mL) and HCl gas was bubbled through the solution. The salt 32 was instantly formed and the methanol was removed under vacuum. The salt was then washed with diethyl ether.

νₘₐₓ (DCM)/cm⁻¹: 2961.78, 1670.62, 1494.19, 1201.18, 1135.92

¹H NMR (CDCl₃, 400 MHz) δ_{H} ppm: 0.99 (6H, t, 2×CH₃ (Leu)), 1.68 (1H, m, CH (Leu)), 1.80 (2H, t, CH₂ (Leu)), 2.72 (2H, d, CH₂), 3.15 (2H, t, CH₂), 3.50 (1H, t, C═OCH (Leu)), 3.62 (3H, s, OCH₃), 3.91 (3H, s, OCH₃), 3.96 (3H, s, OCH₃), 3.97 (3H, s, OCH₃), 6.40 (2H, s, ArH (A-ring) and C═CH), 6.91 (1H, d, ArH (C-ring)), 7.08 (1H, d, ArH (C-ring)), 7.31 (1H, s, ArH (C-ring)), 8.44 (1H, br, NH).

¹³C NMR (CDCl₃, 400 MHz) δ_{c} ppm: 20.0 (CH₂), 21.3 (CH₃ (Leu)), 23.6 (CH₃ (Leu)), 24.9 (CH(CH₃)₂ (Leu)), 44.2 (CH₂ (Leu)), 45.5 (CH₂), 54.34 (CHNH₂), 55.92 (OCH₃), 55.17 (OCH₃), 60.96 (OCH₃), 61.40 (OCH₃), 109.57 (ArCH), 112.15 (ArCH), 120.21 (ArCH), 124.62 (ArCH), 127.33 (ArC), 128.59 (ArCH), 129.22 (ArC), 132.55 (ArC), 135.64 (ArC), 143.32 (ArC), 149.11 (ArC), 150.00 (ArC), 151.00 (ArC), 152.01 (ArC), 173.67 (NHC═O (Leu)), 203.96 (C═O).

### Synthesis of sodium (5-((Z)-6,7-dihydro-2,3,4-trimethoxy-7-oxo-5H-benzo[7]annulen-9-yl)-2-methoxyphenyl)methylphosphonate.disodium salt 33

### Step 1

### Phosphate Ester Synthesis: Synthesis of dibenzyl (5-((Z)-6,7-dihydro-2,3,4-trimethoxy-7-oxo-5H-benzo[7]annulen-9-yl)-2-methoxyphenyl)methylphosphonate 33.1

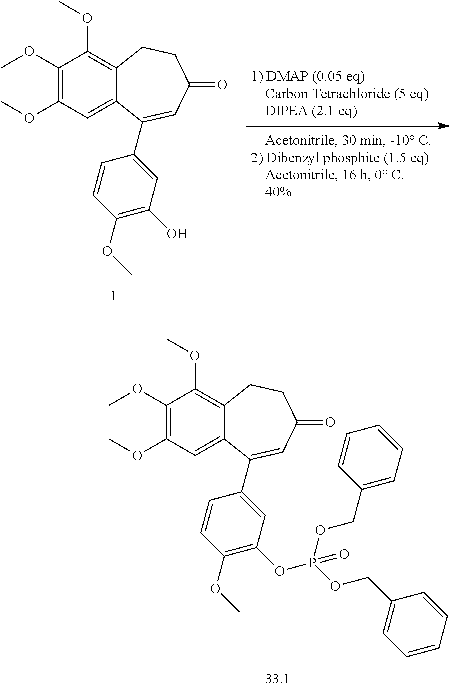 [SMILES: COc1ccc(C2=CC(=O)CCc3c2cc(OC)c(OC)c3OC)cc1O ; COc1ccc(C2=CC(=O)CCc3c2cc(OC)c(OC)c3OC)cc1OP(=O)(OCc1ccccc1)OCc1ccccc1]

To a stirred solution of 1 (0.23 g, 0.62 mmol) in anhydrous acetonitrile (10 mL) under an atmosphere of nitrogen at added DMAP (0.004 g, 0.033 mmol). The temperature was lowered to −10° C. and carbon tetrachloride (0.3 mL, 3.10 mmol) and N,N-diisopropylethylamine (0.23 mL, 1.30 mmol) were added dropwise. After 30 min a solution of dibenzyl phospite (0.24 g, 0.93 mmol) in anhydrous acetonitrile (1 mL) was added dropwise. The temperature of the reaction was allowed to increase to 0° C. and was left stirring at this temperature for 16 h. The reaction was then quenched by the addition of 0.5M monobasic potassium phosphate aqueous solution (50 mL). The reaction mixture was then extracted with diethyl ether (3×50 mL). The organic fractions were dried over MgSO₄, filtered and concentrated in vacuo. The resulting residue was then purified by flash column chromatography (stationary phase; silica gel 230-400 mesh, mobile phase; 10:1, hexane/ethyl acetate) to yield 33.1 as a green oil (0.16 g, 40%).

¹H NMR (CDCl₃, 400 MHz) δ_{H} ppm: 2.73 (2H, m, CH₂), 3.15 (2H, m, CH₂), 3.62 (3H, s, OMe), 3.85 (3H, s, OMe), 3.91 (3H, s, OMe), 3.94 (3H, s, OMe), 5.16 (4H, d, J=8.16 Hz, 2×OCH₂) 6.34 (2H, s, 1×C═CH and 1×ArH), 6.94 (1H, d, J=8.67 Hz, ArH), 7.08 (1H, s, ArH), 6.22 (1H, d, J=9 Hz, ArH)

¹³C NMR (CDCl₃, 400 MHz) δ_{c} ppm: 20.28 (CH₂), 45.62 (CH₂), 55.97 (OMe), 56.01 (OMe), 60.91 (OMe), 61.41 (OMe), 69.69 (CH₂), 69.94 (CH₂), 111.69 (C═CH), 112.24 (ArCH), 122.55 (ArCH), 122.58 (ArCH), 126.78 (ArCH), 127.89 (5×ArCH), 128.47 (ArCH), 128.58 (4×ArCH), 129.19 (QC), 132 (QC), 135.17 (QC), 135.50 (QC), 135.57 (QC), 139.23 (QC), 139.31 (QC), 143.38 (QC), 150.08 (QC), 150.51 (QC), 151.20 (QC), 151.49 (QC), 203.81 (C═O)

vₘₐₓ (DCM)/cm⁻¹: 2091.99, 1650.96, 1512.87, 1493.77, 1454.10

HRMS: calculated 653.1916, found 653.1913, molecular formula (C₃₆H₃₇NaO₉P).

### Step 2

### Removal of Benzyl Groups and Formation of Disodium Salt: Synthesis of sodium (5-((Z)-6,7-dihydro-2,3,4-trimethoxy-7-oxo-5H-benzo[7]annulen-9-yl)-2-methoxyphenyl)methylphosphonate 33

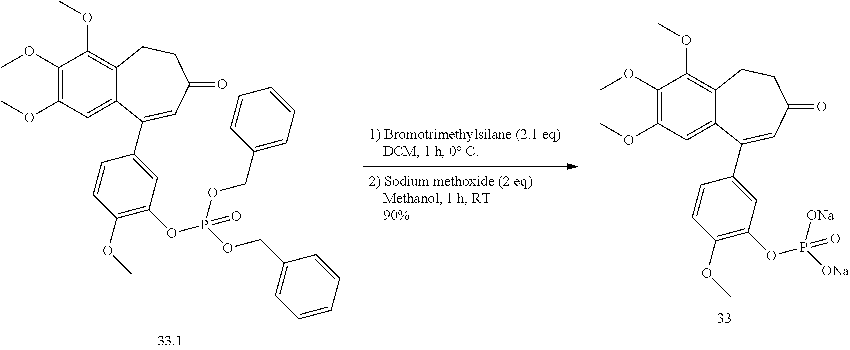

To a stirred solution of 33.1 (0.16 g, 0.25 mmol) in dry DCM under an atmosphere of nitrogen at 0° C. was added bromotrimethylsilane (0.07 mL, 0.52 mmol). After 1 hour the DCM was removed using a stream of nitrogen at room temperature and the resulting residue dissolved in distilled water (50 mL). The aqueous solution was then washed with diethyl ether (5×25 mL) and removed under reduced pressure. This residue was then re-dissolved in methanol (2 mL) and sodium methoxide (0.2 g, 0.37 mmol) was added to the stirred solution. After 1 hour the methanol was removed under reduced pressure to afford the disodium salt 33 as an off white solid (0.8 g, 90%).

¹H NMR (CD₃OD, 400 MHz) δ_{H} ppm: 2.67 (2H, m, CH₂), 3.14 (2H, m, CH₂), 3.64 (3H, s, OMe), 3.90 (6H, s, OMe), 3.91 (3H, s, OMe), 6.43 (1H, s, C═CH), 6.51 (1H, s, ArH), 7 (1H, dd, J₁=2.03 Hz, J₂=8.28 Hz, ArH), 7.03 (1H, d, J=8.48 Hz, ArH), 7.54 (1H, s, ArH)

¹³C NMR (CD₃OD, 400 MHz) δ_{c} ppm: 19.72 (C_H₂), 45.17 (CH₂), 55.03 (OMe), 55.10 (OMe), 59.86 (OMe), 60.50 (OMe), 111.68 (ArCH), 112.07 (ArCH), 121.51 (ArCH), 123.88 (ArCH), 127.12 (C═CH), 128.92 (QC), 132.38 (QC), 134.68 (QC), 142.63 (QC), 143.32 (QC), 149.83 (QC), 151.24 (QC), 152.06 (QC), 152.81 (QC), 205.01 (C═O)

vₘₐₓ (KBr)/cm⁻¹: 3540.22, 2935.47, 1660.91, 1599.01, 1566.73

HRMS: calculated 450.1444, found 450.1064, molecular formula (C₂₂H₂₇O₈P).

Melting point: 166-167° C.

### Synthesis of (5Z,8Z)-8,9-dihydro-5-(3-hydroxy-4-methoxyphenyl)-8-(1-hydroxyethylidene)-1,2,3-trimethoxybenzo[7]annulen-7-one 34

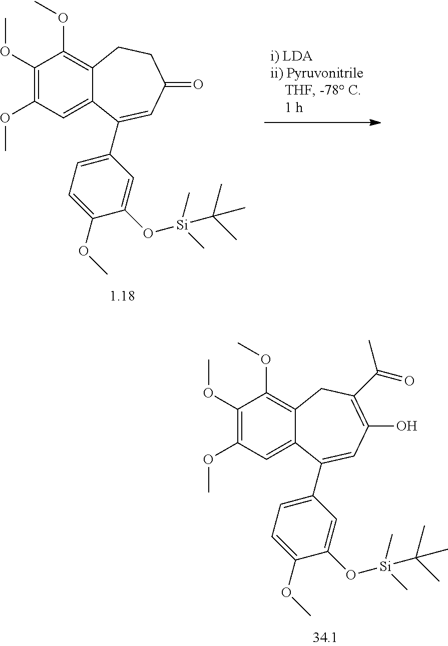 [SMILES: COc1ccc(C2=CC(=O)CCc3c2cc(OC)c(OC)c3OC)cc1O[Si](C)(C)C(C)(C)C ; COc1ccc(C2=CC(O)=C(C(C)=O)Cc3c2cc(OC)c(OC)c3OC)cc1O[Si](C)(C)C(C)(C)C]

### Step 1: Preparation of 0.25 M Lithium Diisopropylamide Solution

To a 100 mL 3 necked round bottom flask was added diisopropylamine (0.18 mL, 1.25 mmol) under an atmosphere of nitrogen to dry THF (4.3 mL). The solution was cooled to −78° C. and stirred for 5 min. 2.5 M nBuLi (0.5 mL, 1.25 mmol) was then added and the solution was stirred at this temperature for 20 min before being allowed to rise to 0° C.

### Step 2 Acylation of 1.18

To a solution of 1.18 (0.07 g, 0.144 mmol) in dry THF (1 mL) at −78° C. was added dropwise a 0.25 M solution of LDA (1.16 mL, 0.29 mmol). The mixture was stirred for 10 min affording a dark red solution. Pyruvonitrile (0.011 g, 0.144 mmol) was dissolved in THF (0.5 mL) and added dropwise to the red solution dropwise over 2 min. After stirring for 1 h at −78° C., the reaction was quenched using 0.5 M HCl (20 mL) and washed with ether (3×15 mL). The combined ether extracts were washed with water (2×20 mL) and dried using MgSO₄. After concentration in vacuo, the product was purified using column chromatography (hexane/EtOAc 8:1) to yield the product 34.1 as a red viscous oil.

Yield: 0.06 g (0.113 mmol, 79%)

¹H NMR (CDCl₃, 400 MHz) δ_{H} ppm: 0.18 (s, 6H, 2×CH₃), 1.01 (s, 9H, tBu), 2.51 (s, 3H, OCH₃), 3.63 (s, 3H, OCH₃), 3.88 (s, 3H, OCH₃), 3.92 (s, 3H, OCH₃), 4.02 (s, 3H, OCH₃), 6.38 (s, 1H), 6.62 (s, 1H), 6.88 (d, J=8.46 Hz, 1H, ArH), 6.94 (d, J=4.46 Hz, 1H, ArH), 7.01 (dd, J=8.36 Hz, 4.8 Hz, 1H, ArH), 15.62 (s, 1H, C═C—OH).

¹³C NMR (CDCl₃, 100.71 MHz) δ_{c} ppm: −4.4 (CH₃), 1.2 (CH₃), 18.6 (Q), 23.7 (CH₂), 25.8 (CH₃), 55.4 (CH₃), 56.0 (CH₃), 60.9 (CH₃), 61.4 (CH₃), 109.3 (CH), 110.7 (Q), 111.5 (CH), 121.9 (CH), 123.0 (CH), 123.4 (CH), 127.7 (Q), 132.1 (Q), 135.0 (Q), 143.7 (Q), 144.7 (Q), 148.7 (Q), 150.9 (Q), 151.6 (Q), 151.7 (Q), 174.83 (C═C—OH), 197.4 (C═O).

MS (+ESI): Calculated Mass 526.2387. Found 525.2319 (M−H)⁻.

### Step 3 Deprotection

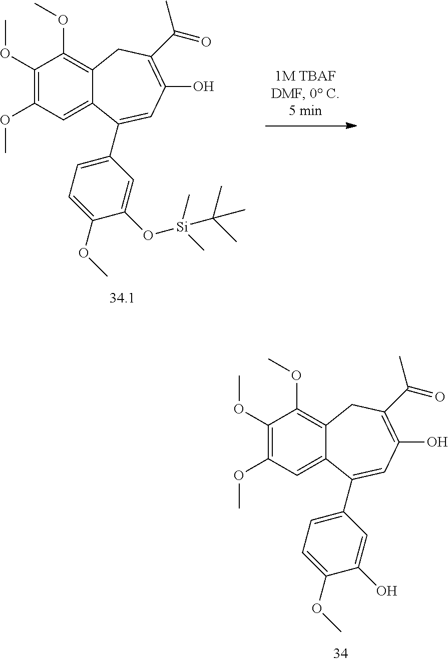 [SMILES: COc1ccc(C2=CC(O)=C(C(C)=O)Cc3c2cc(OC)c(OC)c3OC)cc1O[Si](C)(C)C(C)(C)C ; COc1ccc(C2=CC(O)=C(C(C)=O)Cc3c2cc(OC)c(OC)c3OC)cc1O]

To a stirred solution of 34.1 (0.04 g, 0.075 mmol) in THF (1 mL) was added a 1M TBAF (0.04 mL, 0.075 mmol) in THF. The mixture was stirred for 5 min. Analysis of the reaction by TLC (hexane/ethyl acetate 1:1) indicated complete consumption of starting material. The solvents were removed in vacuo and the remaining residue was placed directly on a column. The product was isolated as a yellow-red solid using a mobile phase system of hexane/ethyl acetate (2:1).

Yield 0.028 g (0.067 mmol, 90%)

¹H NMR (CDCl₃, 400 MHz) δ_{H} ppm: 2.52 (s, 3H, OCH₃), 3.66 (s, 3H, OCH₃), 3.89-3.91 (m, 2H, CH₂), 3.94 (s, 3H, OCH₃), 3.96 (s, 3H, OCH₃), 4.01 (s, 3H, OCH₃), 5.69 (s, 1H, ArOH), 6.41 (s, 1H), 6.65 (s, 1H), 6.89 (d, J=8.32 Hz, 1H, ArH), 6.98-7.02 (m, 2H, 2×ArH), 15.57 (s, 1H, C═C—OH).

¹³C NMR (CDCl₃, 100.71 MHz) δ_{c} ppm: 1.03 (CH), 23.6 (CH₂), 25.6 (CH₃), 56.0 (CH₃), 56.1 (CH₃), 60.9 (CH₃), 61.4 (CH₃), 109.4 (CH), 110.3 (CH), 110.7 (Q), 115.6 (CH), 121.2 (CH), 123.7 (CH), 127.8 (Q), 132.0 (Q), 135.7 (Q), 145.4 (Q), 147.2 (Q), 148.7 (Q), 150.9 (Q), 151.6 (Q), 174.7 (C═C—OH), 197.5 (C═O)

MS (+ESI): Calculated Mass 412.1522. Found 411.1495 (M−H)⁻.

### Synthesis of 3-amino-5-(3-hydroxy-4-methoxyphenyl)-7,8,9-trimethoxy-2,3-dihydro-1-benzoxepin-2-one 35

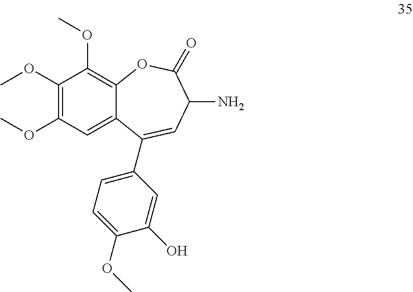 [SMILES: COc1ccc(C2=CC(N)C(=O)Oc3c2cc(OC)c(OC)c3OC)cc1O]

### Step 1

### Synthesis of 3-amino-5-{3[(tert-butyldimethylsilyl)oxy]-4-methoxyphenyl}-7,8,9-trimethoxy-2,3-dihydro-1-benzoxepin-2-one 35.1

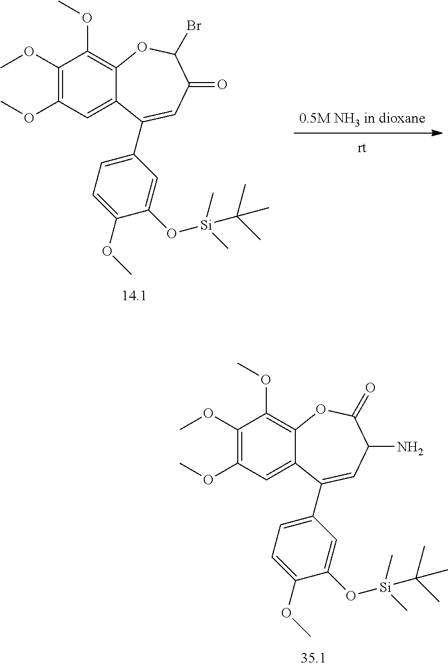 [SMILES: COc1ccc(C2=CC(=O)C(Br)Oc3c2cc(OC)c(OC)c3OC)cc1O[Si](C)(C)C(C)(C)C ; COc1ccc(C2=CC(N)C(=O)Oc3c2cc(OC)c(OC)c3OC)cc1O[Si](C)(C)C(C)(C)C]

2-bromo-5-{3-[tert-butyldimethylsilyl)oxy]-4-methoxyphenyl}-7,8,9-trimethoxy-2,3-dihydro-1-benzoxepin-3-one 14.1 (0.21 g, 0.371 mmol) was stirred with ammonia (0.5 M in dioxane, 3.5 mL, 1.75 mmol) at room temperature for 3 h, after which solvents were removed under reduced pressure. The remaining crude mixture was then loaded onto silica gel and purified via column chromatography (2:1, hexane:ethyl acetate) to afford target compound 2-amino-5-{3-[tert-butyldimethylsilyl)oxy]-4-methoxyphenyl}-7,8,9-trimethoxy-2,3-dihydro-1-benzoxepin-3-one 35.1 (0.12 g, 0.239 mmol, 64%) as a viscous brown oil.

1H NMR (CDCl₃, 400 MHz) δH: 0.19 (6H, s, 2×SiCH₃), 1.02 (9H, s, C(CH₃)₃), 3.70 (3H, s, OCH₃), 3.87 (3H, s, OCH₃), 3.95 (3H, s, OCH₃), 3.99 (3H, s, OCH₃), 5.27 (1H, d, CHNH₂, J=3.5 Hz), 5.63 (1H, br s, NH), 5.96 (1H, d, C═CH, J=3.5 Hz), 6.45 (1H, s, ArCH), 6.78 (1H, br s, NH), 6.89 (2H, m, 2×ArCH), 6.95 (1H, dd, ArCH, J=8.2 Hz, 2.2 Hz)

13C NMR (CDCl₃, 400 MHz) δC: −4.6 (2×SiCH₃), 18.4 (C(CH₃)₃), 25.7 (3×C(CH₃)₃), 55.4 (OCH₃), 56.4 (OCH₃), 61.3 (OCH₃), 61.5 (OCH₃), 74.5 (CNH₂), 105.2 (ArCH), 111.8 (ArCH), 118.1 (ArC), 119.0 (C═CH), 121.1 (ArCH), 122.1 (ArCH), 129.9 (ArC), 136.8 (ArC), 140.1 (ArC), 142.2 (ArC), 143.3 (ArC), 144.8 (ArC), 147.8 (C═C), 151.0 (ArC), 172.2 (e═O)

νₘₐₓ (DCM)/cm⁻¹: 2931.44, 1694.26, 1510.13, 1460.43, 1419.02

HRMS m/z 500.2109, 524.2087 (M+Na), 540.2076 (M+K)

### Step 2

### Synthesis of 3-amino-5-(3-hydroxy-4-methoxyphenyl)-7,8,9-trimethoxy-2,3-dihydro-1-benzoxepin-2-one 33

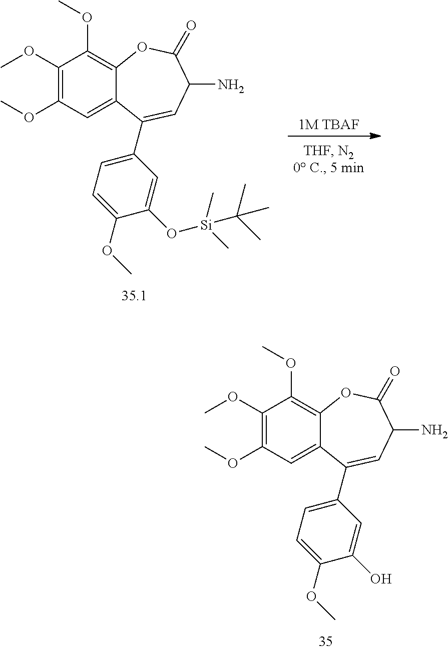

2-amino-5-{3-[tert-butyldimethylsilyl)oxy]-4-methoxyphenyl}-7,8,9-trimethoxy-2,3-dihydro-1-benzoxepin-3-one 33.1 (0.12 g, 0.239 mmol) was dissolved in dry THF (2 mL) under an atmosphere of nitrogen at 0° C. and tetrabutylammonium fluoride (0.26 mL, 1 M solution, 0.263 mmol) added dropwise. After 5 min the reaction was loaded onto silica and purified by column chromatography (1:1 hexane:ethyl acetate) to afford target molecule 3-amino-5-(3-hydroxy-4-methoxyphenyl)-7,8,9-trimethoxy-2,3-dihydro-1-benzoxepin-2-one 33 (0.051 g, 0.131 mmol, 56%) as a yellow solid.

1H NMR (CDCl₃, 400 MHz) δH: 3.72 (3H, s, OCH₃), 3.95 (3H, s, OCH₃), 3.96 (3H, s, OCH₃), 3.99 (3H, s, OCH₃), 5.26 (1H, s, CHNH₂), 5.75 (1H, br s, COH), 5.81 (1H, br s, NH), 5.98 (1H, s, C═CH), 6.48 (1H, s, ArCH), 6.80 (1H, br s, NH), 6.97 (3H, m, 3×ArCH)

13C NMR (CDCl3, 400 MHz) δC: 55.9 (OCH₃), 56.5 (OCH₃), 61.3 (OCH₃), 61.6 (OCH₃), 74.4 (CNH₂), 105.3 (ArCH), 110.6 (ArCH), 114.9 (ArCH), 118.3 (C═CH), 120.4 (ArCH), 122.1 (ArCH), 130.6 (ArC), 136.8 (ArC), 140.1 (ArC), 142.1 (ArC), 143.3 (ArC), 145.5 (ArC), 146.6 (C═C), 147.8 (ArC), 172.3 (C═O)

νₘₐₓ (DCM)/cm⁻¹:

HRMS m/z 386.1248, 410.1222 (M+Na)

Formation of 9-(3-hydroxy-4-methoxyphenyl)-1,2,3-trimethoxy-6,7,8,9-tetrahydro-5Hbenzo[a]cyclohepten-7-one 36

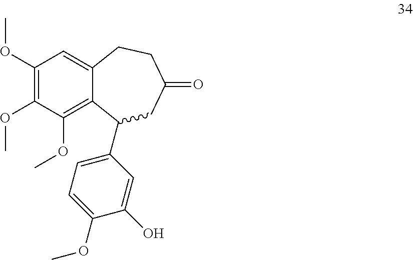 [SMILES: COc1ccc(C2CC(=O)CCc3cc(OC)c(OC)c(OC)c32)cc1O]

### 1^{st} Step

### Synthesis of Intermediate, 9-(3-[1-(tert-butyl)-1,1-dimethylsilyl]oxy-4-methoxyphenyl)-1,2,3-trimethoxy-6,7,8,9-tetrahydro-5H-benzo[a]cyclohepten-7-ol 36.1

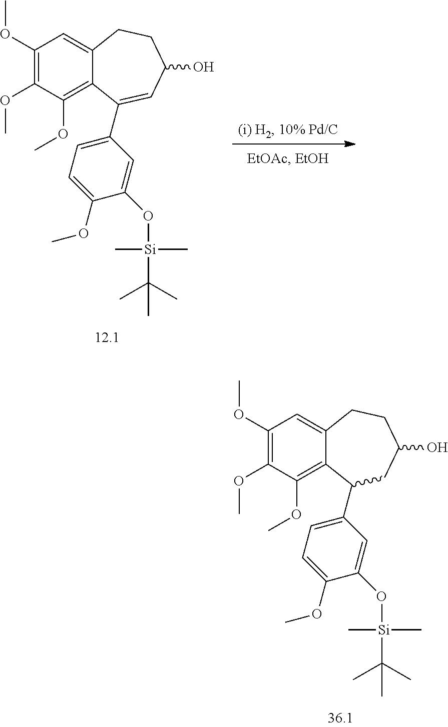 [SMILES: COc1ccc(C2=CC(O)CCc3cc(OC)c(OC)c(OC)c32)cc1O[Si](C)(C)C(C)(C)C ; "(i) H2, 10% Pd/C" ; COc1ccc(C2CC(O)CCc3cc(OC)c(OC)c(OC)c32)cc1O[Si](C)(C)C(C)(C)C]

To a solution of 12.1 (0.1 g, 0.20 mmol) in ethanol/ethyl acetate (1:1, 2 mL) was added 10% Pd/C (0.1 g). The reaction was stirred under a hydrogen atmosphere for 48 h. On completion, the reaction was filtered and the filtrate was concentrated to an oil. It was purified by flash column chromatography (stationary phase: silica gel G₂₅₄; mobile phase: hexane/ethyl acetate 1:1). All homogeneous fractions were collected and the solvent was removed in vacuo to afford 36.1 as a clear oil (0.099 g, 99%). ¹H NMR (CDCl₃, 400 MHz) δ_{H} ppm 0.10 (6H, s CH₃SiCH₃), 0.96 (9H, s, C(CH₃)₃), 1.82 (1H, m, HCH), 1.97 (1H, m, HCH), 2.26 (1H, m, HCH), 2.47-2.59 (2H, m, CH₂), 3.11 (1H, m, HCH), 3.50 (3H, s, OMe), 3.77 (3H, s, OMe), 3.84 (3H, s, OMe), 3.90 (3H, s, OMe), 4.03 (1H, m, CHOH), 4.86 (1H, t, J 6.7 Hz, ArCHAr), 6.54 (1H, s, (A-ring} ArH), 6.65 (1H, s, {C-ring}ArH), 6.71 (1H, d, J=8.0 Hz, {C-ring}ArH), 6.76 (1H, d, J 8.0 Hz, {C-ring}ArH). ¹³C NMR δ_{c} ppm −5.08 (CH₃SiCH₃), 17.96 (2×C(CH₃)₃), 25.26 (2×C(CH₃)₃), 29.86 (CH₂), 34.76 (CH₂), 37.57 (ArCHAr), 39.90 (CH₂), 55.15 (OMe), 55.47 (OMe), 60.27 (OMe), 60.54 (OMe), 70.04 (CHOH), 108.68 (ArCH), 111.77 (ArCH), 119.65 (2×ArCH), 126.71 (qC), 136.69 (qC), 136.81 (qC), 140.29 (qC), 144.51 (qC) 148.71 (qC), 151.07 (qC), 151.70 (qC).

### B 2^{nd} Step-Oxidation and Deprotection

### Synthesis of 9-(3-hydroxy-4-methoxyphenyl)-1,2,3-trimethoxy-6,7,8,9-tetrahydro-5Hbenzo[a]cyclohepten-7-one 36

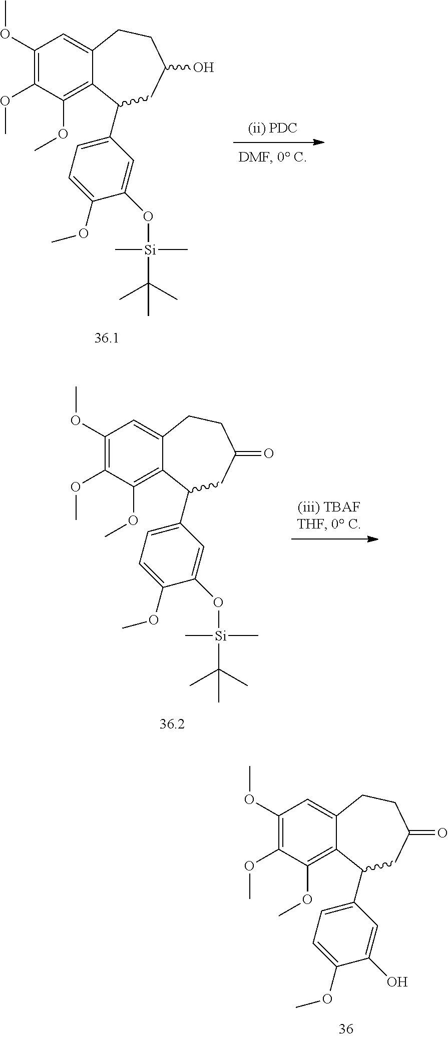 [SMILES: COc1ccc(C2CC(O)CCc3cc(OC)c(OC)c(OC)c32)cc1O[Si](C)(C)C(C)(C)C ; "(ii) PDC" ; COc1ccc(C2CC(=O)CCc3cc(OC)c(OC)c(OC)c32)cc1O[Si](C)(C)C(C)(C)C ; COc1ccc(C2CC(=O)CCc3cc(OC)c(OC)c(OC)c32)cc1O]

To a stirred solution of 36.1 (0.03 g, 0.061 mmol) in DMF (1 mL) was added PDC (0.045 g, 0.119 mmol) over a period of 2 h at 0° C. The reaction was allowed to proceed for 12 h before being quenched by the addition of water (5 mL). The product was then extracted with diethyl ether (5×5 mL) and the organic fractions were collected and dried over sodium sulphate before being concentrated in vacuo to afford 36.1. The ketone 36.2 (0.02 g, 0.041 mmol) was subsequently re-dissolved in THF (1 mL) and 1M TBAF (0.05 mL, 0.050 mmol) was added drop-wise at room temperature. After 1 hour, the reaction was quenched by the addition of water (5 mL) and the product was extracted with diethyl ether (3×5 mL). The ether extracts were combined, dried over sodium sulphate and filtered before the filtrate was concentrated in vacuo. The resulting residue was purified by flash column chromatography (stationary phase; silica gel 230-400 mesh, mobile phase; 1:1 hexane/ethyl acetate). All homogenous fractions were collected and the solvent was evaporated off to afford 36 as a white solid (0.015 g, 99%). M.pt. 44-46° C. HRMS: found 395.1461 (M⁺+Na), requires (C₂₁H₂₄O₆) 372.1573. νₘₐₓ (KBr)/cm⁻¹ 3400.5, 2936.7, 1701.6, 1595.6. ¹H NMR (CD₃Cl, 400 MHz) δ_{H} ppm 2.46 (1H, m, H-6), 2.63 (1H, m, H-5), 2.67 (1H, m, H-6), 2.87 (2H, m, H-8), 2.94 (1H, m, H-5), 3.37 (1H, dd, J 6.8 Hz, 13.8 Hz, H-8), 3.69 (3H, s, OMe), 3.86 (3H, s, OMe), 3.88 (3H, s, OMe), 3.89 (3H, s, OMe), 4.95 (1H, t, J 6.2 Hz, H-9), 5.53 (1H, s, br, —OH), 6.53 (1H, s, H-4), 6.62 (1H, d, J 2.0 Hz, H-2′), 6.67 (1H, dd, J 2 Hz, 8.7 Hz, H-6′), 6.76 (1H, d, J 8.5 Hz, H-5′). ¹³C NMR δ_{c} ppm 29.67 (C-5), 37.04 (C-9), 43.85 (C-6), 47.55 (C-8), 55.46 (OMe), 55.49 (OMe), 60.26 (OMe), 60.60 (OMe), 109.00 (C-4), 110.07 (C-2′), 112.98 (C-6′), 118.02 (C-5′), 126.98 (qC), 131.22 (qC), 135.53 (qC) 136.46 (qC), 144.43 (qC), 145.09 (qC), 151.79 (qC), 210.47 (C═O).

### Hydroxamic Acid Synthesis-Synthesis of 37

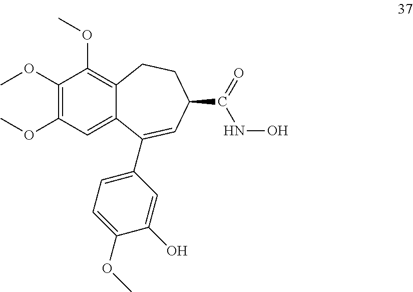 [SMILES: COc1ccc(C2=C[C@H](C(=O)NO)CCc3c2cc(OC)c(OC)c3OC)cc1O]

### Reduction: Synthesis of (7S)-7-((tert-butyldiphenylsilyl)oxy)-1,2,3-trimethoxy-6,7,8,9-tetrahydro-5H-benzo[7]annulen-5-ol 1.9

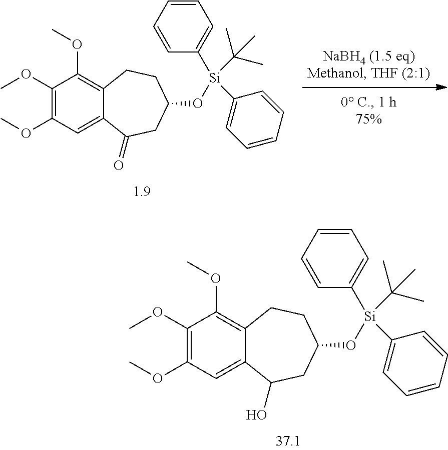 [SMILES: COc1cc2c(c(OC)c1OC)CC[C@H](O[Si](c1ccccc1)(c1ccccc1)C(C)(C)C)CC2=O ; "NaBH4 (1.5 eq)Methanol, THF (2:1)" ; COc1cc2c(c(OC)c1OC)CC[C@H](O[Si](c1ccccc1)(c1ccccc1)C(C)(C)C)CC2O]

To a stirred solution of 1.9 (0.32 g, 0.63 mmol) in methanol (3 mL) and THF (1.5 mL) at 0° C. was added NaBH₄ (0.04 g, 0.95 mmol). After 5 minutes the reaction was quenched by the addition of water (25 mL). The organic solvent was then removed under reduced pressure and the resulting residue extracted with diethyl ether (3×20 mL). The organic fractions were combined and dried over MgSO₄. The solvent was then removed in vacuo to afford 37.1 as a white solid (0.24 g, 75%).

¹H NMR (CDCl₃, 400 MHz) δ_{H} ppm: 1.12 (9H, s, major diastereomer, C(CH₃)₃), 1.17 (9H, s, minor diastereomer C(CH₃)₃), 1.33-1.43 (1H, m, minor diastereomer, CH₂), 1.64 (2H, m, minor and major diastereomers, CH₂), 1.69 (1H, s, minor diastereomer, CH₂), 1.73-1.83 (2H, m, minor and major disastereomers, CH₂), 1.86-1.96 (1H, major diastereomer, CH₂), 1.96-2.05 (2H, m, minor and major diastereomers, CH₂), 2.21 (1H, br s, major diastereomer, OH), 2.45 (1H, br s, minor diastereomer, OH), 2.98-3.02 (2H, m, minor and major diastereomers, CH₂), 3.24-3.30 (1H, m, minor and major diastereomers, CH₂), 3.80 (3H, s, major diastereomer, OMe), 3.81 (3H, s, minor diastereomer, OMe), 3.87 (3H, s, major diastereomer, OMe), 3.88 (3H, s, minor diastereomer, OMe), 3.88 (3H, s, major diastereomer, OMe), 3.89 (3H, s, minor diastereomer, OMe), 4.22 (1H, s br, major diastereomer, CH), 4.30 (1H, m, minor diastereomer, CH), 4.70 (1H, m, major diastereomer, CH), 5.43 (1H, m, minor diastereomer, CH), 6.78 (1H, s, major diastereomer, ArH), 6.95 (1H, s, minor diastereomer, ArH), 7.41-7.52 (6H, m, minor and major diastereomers, ArH), 7.72-7.80 4H, m, minor and major diastereomers, ArH).

¹³C NMR (CDCl₃, 400 MHz) δ_{c} ppm: 17.70 (CH₂), 18.29 (CH₂), 19.16 (C(CH₃)₃), 19.44 (C(CH₃)₃), 26.98 (C(CH₃)₃), 27.13 (C(CH₃)₃), 34.99 (CH₂), 35.47 (CH₂), 44.80 (CH₂), 55.99 (OMe), 56.01 (OMe), 60.87 (OMe), 60.89 (OMe), 61.37 (OMe), 69.55 (CHOSi), 73.86 (CHOH), 103.71 (ArCH), 125.78 (ArC), 127.68 (2×ArCH), 127.72 (2×ArCH), 129.74 (ArCH), 129.79 (ArCH), 129.87 (ArCH), 129.90 (ArCH), 133.64 (ArC), 134.23 (ArC), 134.55 (ArC), 134.82 (ArC), 135.85 (2×ArCH), 135.90 (2×ArCH), 139.49 (ArC), 140.59 (ArC), 141.00 (ArC), 141.11 (ArC), 150.94 (ArC), 151.03 (ArC), 151.07 (ArC), 151.11 (ArC)

vₘₐₓ (DCM)/cm⁻¹:

HRMS: calculated 529.2386, found 529.4425, molecular formula (C₃₀H₃₈O₅SiNa).

Melting point: 57-59° C.

### MOM Protection: Synthesis of ((7S)-6,7,8,9-tetrahydro-1,2,3-trimethoxy-5-(methoxymethoxy)-5H-benzo[7]annulen-7-yloxy)(tert-butyl)diphenylsilane 37.2

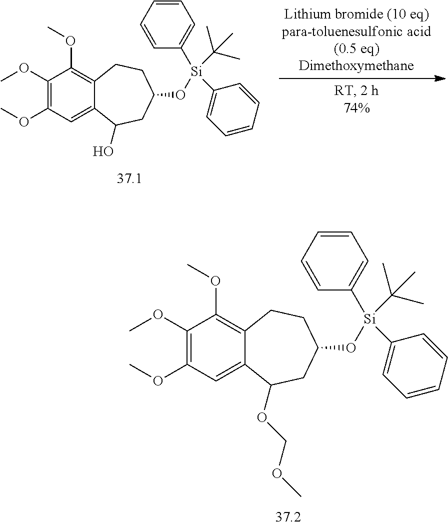

To stirred solution of 37.1 (0.1 g, 0.2 mmol) in dimethoxymethane (2 mL) was added lithium bromide (0.17 g, 1.97 mmol) and para-toluenesulfonic acid (0.02 g, 0.1 mmol). After 2 hours the reaction was quenched by the addition of water (10 mL). The reaction mixture was then extracted with diethyl ether (3×20 mL). The organic fractions were combined, dried over MgSO₄ and concentrated in vacuo. The resulting residue was then purified by flash column chromatography (stationary phase; silica gel 230-400 mesh, mobile phase; 10:1, hexane/ethyl acetate) to yield 37.2 as a clear oil (80 mg, 74%).

¹H NMR (CDCl₃, 400 MHz) δ_{H} ppm: 1.08 (9H, s, major diastereomer, C(CH₃)₃), 1.17 (9H, s, minor diastereomer C(CH₃)₃), 1.28-1.39 (2H, m, minor and major diastereomers, CH₂), 1.65-1.78 (2H, m, minor and major diastereomers, CH₂), 1.89-2.05 (3H, m, minor and major diastereomers, CH₂), 2.15-2.24 (2H, m, major diastereomer, CH₂), 2.83 (1H, s, minor diastereomer, CH₂), 3.00 (1H, m, minor diastereomer, CH₂), 3.13-3.22 (1H, q, J=7.16 Hz, major diastereomer, CH₂), 3.25 (3H, s, major diastereomer, OMe), 3.35 (3H, s, minor diastereomer, OMe), 3.80 (3H, s, minor diastereomer, OMe), 3.82 (3H, s, major diastereomer, OMe), 3.84-3.92 (1H, m, minor diastereomer, CHOSi), 3.87 (6H, s, major and minor diastereomers, OMe), 3.90 (6H, s, minor and major diastereomers, OMe), 3.94-4.05 (1H, m, major diastereomer, CHOSi), 4.32 (1H, s, br, minor diastereomer, CHOCH₂), 4.44 (1H, d, major diastereomer, CHOCH₂) 4.51 (H, d, J=6.6 HZ, major diastereomer, CHOCH₂OCH₃), 4.55 (H, d, J=6.6 HZ, major diastereomer, CHOCH₂OCH₃), 4.61 (2H, br s, minor diastereomer, CHOCH₂OCH₃), 6.79 (1H, s, minor diastereomer, ArH), 6.87 (1H, s, major diastereomer, ArH), 7.38-7.50 (6H, m, minor and major diastereomers, ArH), 7.70-7.80 (4H, m, minor and major diastereomers, ArH).

¹³C NMR (CDCl₃, 400 MHz) δ_{c} ppm: 18.14 (CH₂), 19.17 (C(CH₃)₃), 19.29 (CH₂), 19.35 (C(CH₃)₃), 26.95 (C(CH₃)₃), 270.09 (C(CH₃)₃), 34.41 (CH₂), 42.55 (CH₂), 44.73 (CH₂), 55.46 (OMe), 55.56 (OMe), 56.00 (OMe), 60.87 (OMe), 61.34 (OMe), 61.39, 69.89 (CHOCH₂), 70.89 (CHOCH₂), 73.49 (CHOSi), 94.44 (OCH₂OCH₃), 103.14 (ArCH), 124.89 (ArC), 127.62 (2×ArCH), 129.65 (2×ArCH), 134.36 (ArC), 134.38 (ArC), 134.41 (ArC), 134.51 (ArC), 135.83 (2×ArCH), 135.87 (2×ArCH), 135.89 (2×ArCH), 138.07 (ArC), 140.71 (ArC), 150.86 (ArC), 150.89 (ArC), 151.10 (ArC), 151.42 (ArC).

vₘₐₓ (DCM)/cm⁻¹:

HRMS: calculated 573.2648, found 573.2669, molecular formula (C₃₂H₄₂NaO₆Si).

### Silyl Deprotection: Synthesis of (7S)-6,7,8,9-tetrahydro-1,2,3-trimethoxy-5-(methoxymethoxy)-5H-benzo[7]annulen-7-ol 37.3

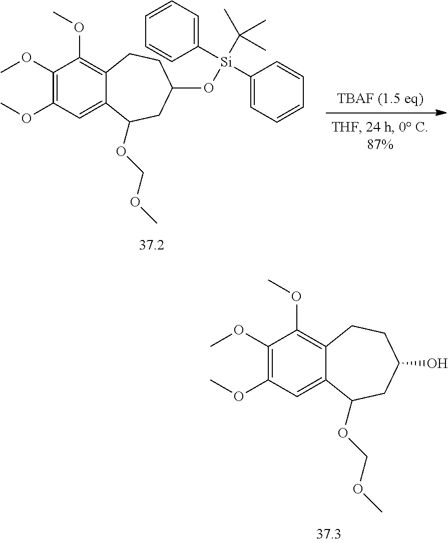 [SMILES: COCOC1CC(O[Si](c2ccccc2)(c2ccccc2)C(C)(C)C)CCc2c1cc(OC)c(OC)c2OC ; "TBAF (1.5 eq)" ; COCOC1C[C@@H](O)CCc2c1cc(OC)c(OC)c2OC]

To a stirred solution of 37.2 (2.65 g, 4.81 mmol) in dry THF under an atmosphere of nitrogen at 0° C. was added 1M tetrabutylammonium fluoride solution (7.22 mL, 7.22 mmol) dropwise. After 24 hours the reaction was quenched by the addition of water (50 mL). The reaction mixture was then extracted with diethyl ether (3×20 mL). The organic fractions were combined, dried over MgSO₄ and concentrated in vacuo. The product was purified by flash column chromatography (stationary phase; silica gel 230-400 mesh, mobile phase; 1:1, hexane/ethyl acetate) to yield 37.3 as a yellow oil (1.31 g, 87%).

¹H NMR (CDCl₃, 400 MHz) δ_{H} ppm: 1.52-2.31 (8H, m, minor and major diastereomers, CH₂), 2.56 (1H, s, br, major diastereomer CH₂), 2.71-2.81 (1H, minor diastereomer CH₂), 2.93-3.04 (1H, m, minor diastereomer, CH₂), 3.11-3.21 (1H, m, major diastereomer, CH₂), 3.37 (3H, s, minor diastereomer, OMe), 3.39 (3H, s, major diastereomer, OMe), 3.78 (3H, s, minor diastereomer, OMe), 3.79 (3H, s, major diastereomer, OMe), 3.83 (6H, s, minor and major diastereomers, 2×OMe), 3.86 (3H, s, minor diastereomer, OMe), 3.87 (3H, s, major diastereomer, OMe), 4.04-4.13 (1H, m, major diastereomer, CHOSi), 4.21-4.30 (1H, m, minor diastereomer, CHOSi), 4.55-4.68 (4H, m, minor and major diastereomers, 2×OCH₂OCH₃), 4.71-4.77 (1H, m, major diastereomer, CHOCH₂O), 4.88-4.95 (1H, m, minor diastereomer, CHOCH₂O), 6.68 (1H, s, minor diastereomer, ArH), 6.70 (1H, s, major diastereomer, ArH)

¹³C NMR (CDCl₃, 400 MHz) δ_{c} ppm: 18.41 (CH₂), 18.74 (CH₂), 35.73 (CH₂), 36.28 (CH₂), 41.79 (CH₂), 55.44 (OMe), 55.64 (OMe), 56.01 (OMe), 60.83 (OMe), 61.36 (OMe), 68.76 (CHOSi), 71.58 (CHOSi), 94.09 (OCH₂OCH₃), 94.21 (OCH₂OCH₃), 107.49 (ArCH), 127.20 (ArC), 136.12 (ArC), 136.75 (ArC), 141.40 (ArC), 150.79 (ArC), 150.91 (ArC), 151.24 (ArC), 151.27 (ArC)

vₘₐₓ (DCM)/cm⁻¹:

HRMS: calculated 335.1471, found 335.1463, molecular formula (C₁₆H₂₄NaO₆).

### Nitrile Synthesis: Synthesis of (7R)-6,7,8,9-tetrahydro-1,2,3-trimethoxy-5-(methoxymethoxy)-5H-benzo[7]annulene-7-carbonitrile 37.5

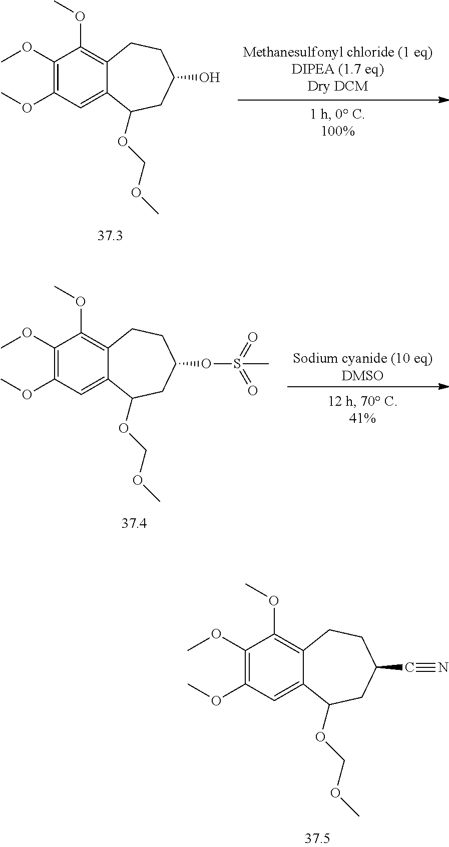

To a stirred solution of 37.3 (2.99 g, 9.58 mmol) in dry DCM (10 mL) under an atmosphere of nitrogen at 0° C. was added methanesulfonyl chloride (1.26 mL, 16.28 mmol) followed by N,N-diisopropylethylamine (2.5 mL, 14.36 mmol). After 1 hour the reaction was quenched by the addition of water (20 mL). The reaction mixture was then extracted with diethyl ether (3×25 mL). The organic fractions were combined and dried over MgSO₄ and concentrated in vacuo to yield the mesylate 37.4 as a crude mixture. The mesylate was dissolved in DMSO (10 mL) at room temperature and sodium cyanide (4.96 g, 10.70 mmol) was added to the solution. The reaction was then refluxed at 70° C. for 24 hours. The reaction was then quenched by the addition of water (30 mL) and extracted with diethyl ether (3×30 mL). The organic fractions were combined, dried over MgSO₄, filtered and concentrated under reduced pressure. The product was purified by flash column chromatography (stationary phase; silica gel 230-400 mesh, mobile phase; 4:1, hexane/ethyl acetate) to yield 37.5 as a yellow oil (1.27 g, 41% over two steps).

¹H NMR (CDCl₃, 400 MHz) δ_{H} ppm: 1.63-1.78 (1H, m, CH₂), 1.96-1.78 (1H, m, CH₂), 2.06-2.19 (1H, m, CH₂), 2.27-2.40 (1H, m, CH₂), 2.77-2.89 (1H, m, CH₂), 2.99-3.11 (1H, m, CH₂), 3.39-3.37 (2H, m, 1×CHCN, 1×OMe), 3.75 (3H, s, OMe), 3.81 (3H, s, OMe), 3.83 (3H, s, OMe), 4.54 (2H, s, OCH₂OCH₃), 4.76 (1H, d, J=7.60 Hz, CHOCH₂), 6.59 (1H, s, ArH)

¹³C NMR (CDCl₃, 400 MHz) δ_{c} ppm: 22.21 (CH₂), 28.14 (CHCN), 31.17 (CH₂), 36.10 (CH₂), 55.50 (OMe), 56.00 (OMe), 60.72 (OMe), 61.32 (OMe), 75.37 (CHOCH₂), 93.93 (OCH₂OCH₃), 108.73 (ArCH), 122.56 (CN), 135.20 (ArC), 141.84 (ArC), 151.06 (ArC), 151.48 (ArC)

vₘₐₓ (DCM)/cm⁻¹:

HRMS: calculated 344.1474, found 344.1465, molecular formula (C₁₇H₂₃NNaO₅).

### Reduction of Nitrile to Alcohol: Synthesis of a7R)-6,7,8,9-tetrahydro-1,2,3-trimethoxy-5-(methoxymethoxy)-5H-benzo[7]annulen-7-yl)methanol 37.7

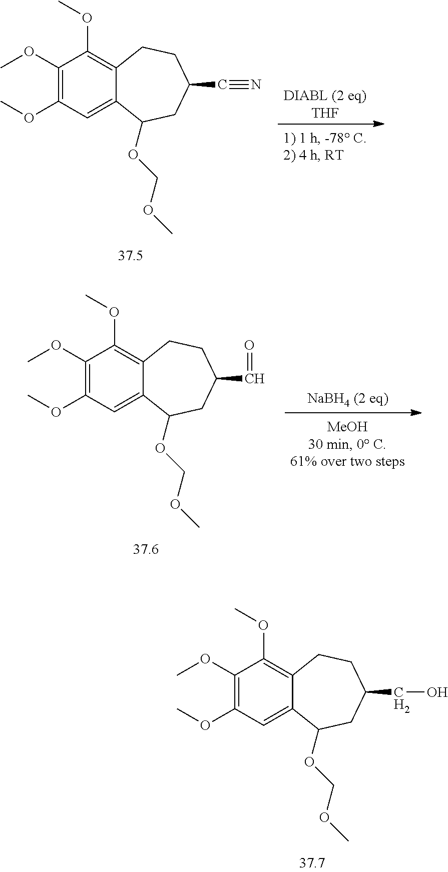 [SMILES: COCOC1C[C@H](C#N)CCc2c1cc(OC)c(OC)c2OC ; "DIABL (2 eq)THF" ; COCOC1C[C@H](C=O)CCc2c1cc(OC)c(OC)c2OC ; "NaBH4 (2 eq)" ; COCOC1C[C@H](CO)CCc2c1cc(OC)c(OC)c2OC]

To a stirred solution of 37.5 (0.08 g, 0.25 mmol) in dry THF (1 mL) under an atmosphere of nitrogen at −78° C. was added DIBAL 1M solution in hexane (0.5 mL, 0.5 mmol) dropwise. After 1 hour the temperature was allowed to increase to ambient. After a further 4 hours the reaction was quenched by the addition of tartaric acid 1M solution (5 mL). The reaction mixture was then extracted with diethyl ether (3×10 mL). The organic fractions were combined and dried over MgSO₄, filtered and concentrated in vacuo to yield the aldehyde 37.6 as a crude mixture. The aldehyde was dissolved in methanol (5 mL) and the temperature of the reaction reduced to 0° C. Sodium borohydride (0.02 g, 0.5 mmol) was slowly added to the reaction. After 30 minutes the methanol was removed under reduced pressure and the reaction was quenched by the addition of water (5 mL). The reaction was then extracted with diethyl ether (3×10 mL). The organic fractions were combined, dried over MgSO₄, filtered and concentrated under reduced pressure. The product was purified by flash column chromatography (stationary phase; silica gel 230-400 mesh, mobile phase; 4:1, hexane/ethyl acetate) to yield 37.7 as a yellow oil (0.05 g, 61% over two steps).

¹H NMR (CDCl₃, 400 MHz) δ_{H} ppm: 1.49-2.08 (3H, m, 2×CH₂, 1×CHCH₂OH), 2.17-2.36 (2H, m, CH₂), 2.77-2.89 (1H, m, CH₂), 3.07-3.17 (1H, m, CH₂), 3.38 (3H, s, OMe), 3.52 (2H, m, CH₂OH), 3.80 (3H, s, OMe), 3.85 (3H, s, OMe), 3.88 (3H, s, OMe), 4.58 (2H, s, OCH₂OCH₃), 4.77 (1H, d, J=6.84 Hz, CHOCH₂), 6.59 (1H, s, ArH)

¹³C NMR (CDCl₃, 400 MHz) δ_{c} ppm: 22.07 (CH₂), 30.09 (CH₂), 34.97 (CH₂), 38.45 (CHCH₂OH), 54.91 (OMe), 55.59 (OMe), 60.39 (OMe), 60.94 (OMe), 68.15 (CH₂OH), 76.85 (CHOCH₂), 93.41 (OCH₂OCH₃), 108.90 (ArCH), 127.96 (ArC), 135.41 (ArC), 141.23 (ArC), 150.02 (ArC), 150.93 (ArC)

vₘₐₓ (DCM)/cm⁻¹:

HRMS: calculated 349.1627, found 349.1633, molecular formula (C₁₇H₂₆NaO₆).

### Silyl Protection: Synthesis of (((7R)-6,7,8,9-tetrahydro-1,2,3-trimethoxy-5-(methoxymethoxy)-5H-benzo[7]annulen-7-yl)methoxy)(tert-butyl)diphenylsilane 37.8

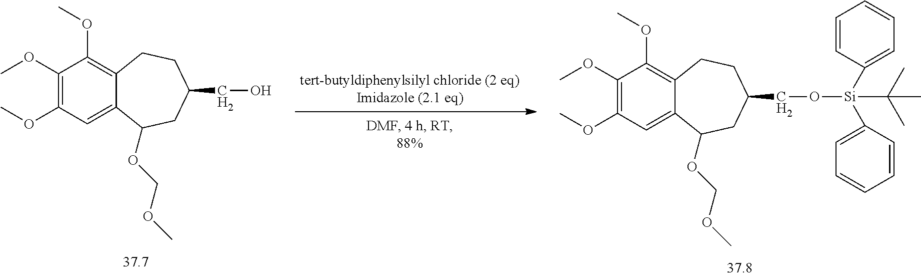 [SMILES: COCOC1C[C@H](CO)CCc2c1cc(OC)c(OC)c2OC ; "tert-butyldiphenylsilyl chloride (2 eq)Imidazole (2.1 eq)" ; COCOC1C[C@H](CO[Si](c2ccccc2)(c2ccccc2)C(C)(C)C)CCc2c1cc(OC)c(OC)c2OC]

To a stirred solution of 37.7 (0.21 g, 0.64 mmol) and imidazole (0.09 g, 1.35 mmol) in dry DMF (1 mL) under an atmosphere of nitrogen at room temperature was added tert-butyldiphenylsilyl chloride (0.33 ml, 1.29 mmol). After 4 hours the reaction was quenched by the addition of water (10 mL) and extracted with diethyl ether (3×20 mL). The organic fractions were combined, dried over MgSO₄, filtered and concentrated under reduced pressure. The product was purified by flash column chromatography (stationary phase; silica gel 230-400 mesh, mobile phase; 20:1, hexane/ethyl acetate) to yield 37.8 as a clear oil (0.32 g, 88%).

¹H NMR (CDCl₃, 400 MHz) δ_{H} ppm: 1.12 (9H, s, C(CH₃)₃), 1.46-1.64 (1H, m, CHCH₂OSi), 2.21-2.48 (4H, m, CH₂), 2.80-2.93 (1H, m, CH₂), 3.11-3.26 (1H, m, CH₂), 3.42 (3H, s, OMe), 3.57 (2H, s, CH₂OSi), 3.85 (3H, s, OMe), 3.90 (3H, s, OMe), 3.94 (3H, s, OMe), 4.64 (2H, s, OCH₂OCH₃), 4.82 (1H, s, CHOCH₂), 6.64 (1H, s, ArH), 7.45 (6H, s, ArH). 7.73 (4H, s, ArH)

¹³C NMR (CDCl₃, 400 MHz) δ_{c} ppm: 18.89 (C(CH₃)₃), 22.30 (CH₂), 26.45 (C(CH₃)₃), 30.35 (CH₂), 35.06 (CH₂), 38.47 (CHCH₂OSi), 54.85 (OMe), 55.59 (OMe), 60.42 (OMe), 60.95 (OMe), 68.90 (CH₂OSi), 77.10 (CHOCH₂), 93.36 (OCH₂OCH₃), 109.04 (ArCH), 127.19 (4×ArCH), 128.33 (ArC), 129.13 (2×ArCH), 133.42 (4×ArC), 135.20 (4×ArCH), 135.68 (4×ArC), 141.27 (2×ArC), 149.97 (3×ArC), 151.01 (3×ArC)

vₘₐₓ (DCM)/cm⁻¹:

HRMS: calculated 587.2805, found 587.2816, molecular formula (C₃₃H₄₄NaO₆Si).

### MOM Deprotection and Oxidation of Alcohol: Synthesis of (7R)-7-[(tert-butyldiphenylsilyl)oxy]-1,2,3-trimethoxy-6,7,8,9-tetrahydro-5H-benzo[7]annulen-5-ol 37.10

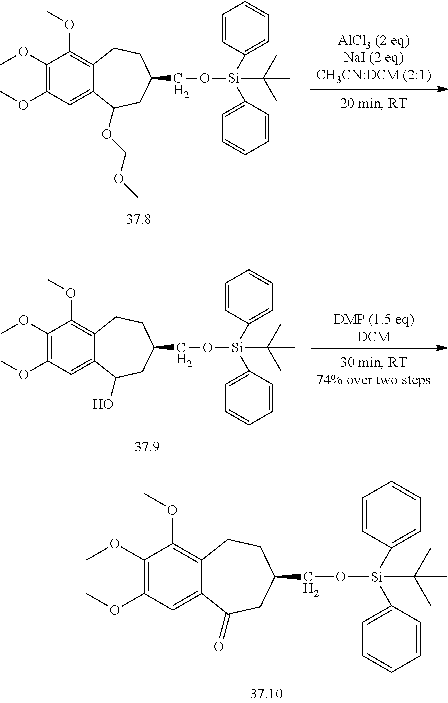

To a stirred solution of 37.8 (0.32 g, 0.57 mmol) in a mixture of acetonitrile and DCM (2:1, 10 mL) at room temperature was added aluminium chloride (0.15 g, 1.14 mmol) and sodium iodide (0.17 g, 1.14 mmol). After twenty minutes the reaction was quenched by the addition of saturated sodium bicarbonate solution. The reaction was then extracted with DCM (3×20 mL). To a stirred solution of the combined organic fractions was added Dess-Martin Periodinane (0.36 g, 0.85 mmol). After thirty minutes the reaction was quenched by the addition of saturated sodium bicarbonate solution and the reaction was extracted with DCM (3×50 mL). The organic fractions were combined, dried over MgSO₄, filtered and concentrated under reduced pressure. The product was purified by flash column chromatography (stationary phase; silica gel 230-400 mesh, mobile phase; 10:1, hexane/ethyl acetate) to yield 37.10 as a yellow oil (0.22 g, 74% over two steps). R_{f}:

¹H NMR (CDCl₃, 400 MHz) δ_{H} ppm: 1.11 (9H, s, C(CH₃)₃), 1.70-1.81 (1H, m, CH₂), 1.85-1.98 (1H, m, CH₂), 2.11-2.29 (1H, m, CHCH₂OSi), 2.71-2.87 (2H, m, CH₂), 2.92 (1H, d, J=14.7 Hz, CH₂), 3.19 (1H, d, J=14.7 Hz, CH₂), 3.60-3.68 (1H, m, CH₂), 3.77-3.85 (1H, m, CH₂), 3.87 (3H, s, OMe), 3.92 (3H, s, OMe), 3.98 (3H, s, OMe), 7.22 (1H, s, ArH), 7.35-7.53 (6H, s, ArH), 7.65-7.81 (4H, s, ArH)

¹³C NMR (CDCl₃, 400 MHz) δ_{c} ppm: 18.86 (C(CH₃)₃), 21.87 (CH₂), 26.43 (C(CH₃)₃), 28.06 (CH₂), 35.54 (CHCH₂OSi), 43.60 (CH₂), 55.54 (OMe), 60.46 (OMe), 60.90 (OMe), 66.68 (CHCH₂OSi), 107.03 (ArCH), 127.28 (4×ArCH), 129.26 (2×ArCH), 133.02 (4×ArC), 133.09 (4×ArC), 133.37 (2×ArC), 133.77 (ArC), 134.89 (ArC), 135.15 (2×ArCH), 135.17 (2×ArCH), 141.04 (ArC), 145.38 (ArC), 150.52 (ArC), 151.10 (ArC), 202.75 (C═O)

vₘₐₓ (DCM)/cm⁻¹:

HRMS: calculated 541.2386, found 541.2339, molecular formula (C₃₁H₃₈NaO₅Si).

### Removal of Silyl Protecting Group: Synthesis of (R)-6,7,8,9-tetrahydro-7-(hydroxymethyl)-1,2,3-trimethoxybenzo[7]annulen-5-one 37.11

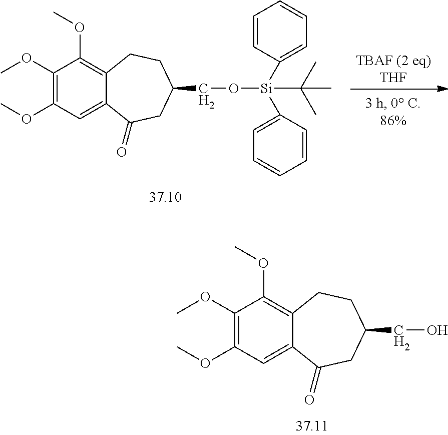

To a stirred solution of 37.10 (0.6 g, 1.16 mmol) in dry THF (5 mL) under an atmosphere of nitrogen at 0° C. was added TBAF 1M solution in THF (2.3 mL, 2.31 mmol) dropwise. After 3 hours the reaction was quenched by the addition of water (25 mL) and extracted with diethyl ether (3×30 mL). The organic fractions were combined, dried over MgSO₄, filtered and concentrated under reduced pressure. The product was purified by flash column chromatography (stationary phase; silica gel 230-400 mesh, mobile phase; 1:1, hexane/ethyl acetate) to yield 37.11 as a clear resin (0.28 g, 86%).

¹H NMR (CDCl₃, 400 MHz) δ_{H} ppm: 1.50-1.61 (1H, m, CH₂), 1.89-2.01 (1H, m, CH₂), 2.07-2.18 (1H, m, CHCH₂OH), 2.65-2.74 (1H, m, CH₂), 2.83-2.94 (1H, m, CH₂), 3.03-3.12 (1H, m, CH₂), 2.47-3.58 (1H, m, CHCH₂OH), 3.58-3.65 (1H, m, CHCH₂OH), 3.82 (3H, s, OMe), 3.87 (3H, s, OMe), 3.91 (3H, s, OMe), 7.14 (ArH)

¹³C NMR (CDCl₃, 400 MHz) δ_{c} ppm: 22.03 (CH₂), 28.05 (CH₂), 35.92 (CHCH₂OH), 43.14 (CH₂), 55.50 (OMe), 60.42 (OMe), 60.83 (OMe), 65.44 (CH₂OH), 106.94 (ArCH), 129.72 (ArC), 133.56 (ArC), 145.40 (ArC), 150.53 (ArC), 151.05 (ArC), 202.78 (C═O)

vₘₐₓ (DCM)/cm⁻¹:

HRMS: calculated 303.1208, found 303.1232, molecular formula (C₁₅H₂₀NaO₅)

### Organolithium C-Ring Coupling: Synthesis of [(7R)-9-{3-[tert-butyldimethylsilyl)oxy]-4-methoxyphenyl}-2,3,4-trimethoxy-6,7-dihydro-5H-benzo[7]annulen-7-yl]methanol 37.12

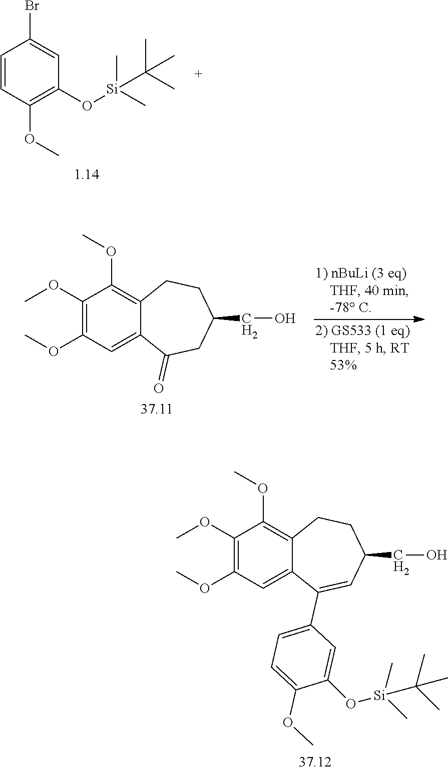 [SMILES: COc1ccc(Br)cc1O[Si](C)(C)C(C)(C)C ; COc1cc2c(c(OC)c1OC)CC[C@@H](CO)CC2=O ; COc1ccc(C2=C[C@H](CO)CCc3c2cc(OC)c(OC)c3OC)cc1O[Si](C)(C)C(C)(C)C]

To a stirred solution of 1.14 (1.15 g, 3.64 mmol) in dry THF (10 mL) at −78° C. under an atmosphere of nitrogen was added nBuLi 2.5M solution in hexane (1.46 mL, 3.64 mmol) dropwise. After 40 minutes a solution of 37.11 (0.34 g, 1.21 mmol) in dry THF (5 mL) was added to the reaction dropwise. The reaction was allowed to increase to ambient and was subsequently remained stirring for 5 hours. The reaction was then quenched by the addition of 2M HCl solution (25 mL) and was extracted with diethyl ether (3×50 mL). The organic fractions were combined, dried over MgSO₄, filtered and concentrated under reduced pressure. The product was purified by flash column chromatography (stationary phase; silica gel 230-400 mesh, mobile phase; 4:1, hexane/ethyl acetate) to yield 37.12 as a yellow oil (0.32 g, 53%).

R_{f}: 0.29 (3:1, hexane/ethyl acetate)

[α]_{D}²⁵=+47.26

¹H NMR (CDCl₃, 400 MHz) δ_{H} ppm: 0.15 (6H, d, J=4.56 Hz, Si(CH₃)₂), 0.99 (9H, s, C(CH₃)₃), 1.86-1.98 (1H, m, CH₂), 2.13-2.41 (3H, m, 1×CHCH₂OH, 2×CH₂), 3.04-3.13 (1H, m, CH₂), 3.70 (5H, s, 1×OMe, 1×CH₂OH), 3.83 (3H, s, OMe), 3.92 (3H, s, OMe), 3.94 (3H, s, OMe), 6.15 (1H, d, J=6.21 Hz, C═CH), 6.38 (1H, s, ArH), 6.78-6.82 (2H, m, ArH), 6.88-6.90 (1H, m, ArH)

¹³C NMR (CDCl₃, 400 MHz) δ_{c} ppm: −4.99 (Si(CH₃)₂)_{,} 18.00 (C(CH₃)₃), 22.69 (CH₂), 25.28 (C(CH₃)₃), 37.46 (CH₂), 39.49 (CHCH₂OH), 50.04 (OMe), 55.44 (OMe), 60.44 (OMe), 61.15 (OMe), 65.94 (CH₂OH), 108.34 (ArCH), 111.12 (ArCH), 120.23 (ArCH), 120.90 (ArCH), 127.62 (ArCH), 127.82 (C═CH), 134.30 (ArC), 135.77 (ArC), 140.68 (ArC), 141.58 (ArC), 144.08 (ArC), 149.95 (ArC), 150.30 (ArC), 150.52 (ArC)

vₘₐₓ (DCM)/cm⁻¹:

HRMS: calculated 501.2672, found 501.2668, molecular formula (C₂₈H₄₁O₆Si).

### Oxidation of Alcohol to Aldehyde: Synthesis of (7R)-9-{3-[(tert-butyldimethylsilyl)oxy]-4-methoxyphenyl}-2,3,4-trimethoxy-6,7-dihydro-5H-benzo[7]annulene-7-carbaldehyde 37.13

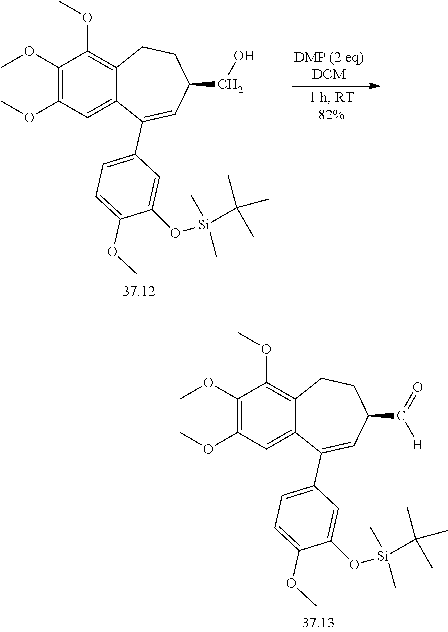 [SMILES: COc1ccc(C2=C[C@H](CO)CCc3c2cc(OC)c(OC)c3OC)cc1O[Si](C)(C)C(C)(C)C ; COc1ccc(C2=C[C@H](C=O)CCc3c2cc(OC)c(OC)c3OC)cc1O[Si](C)(C)C(C)(C)C]

To a stirred solution of 37.12 (0.11 g, 0.22 mmol) in DCM (5 mL) at room temperature was added Dess-Martin Periodinane (0.19 g, 0.44 mmol). After 1 hour the reaction was quenched by the addition of saturated sodium bicarbonate solution (10 mL). The reaction was extracted with diethyl ether (3×20 mL). The organic fractions were combined, dried over MgSO₄, filtered and concentrated under reduced pressure. The product was purified by flash column chromatography (stationary phase; silica gel 230-400 mesh, mobile phase; 10:1, hexane/ethyl acetate) to yield 37.13 as a yellow oil (0.09 g, 82%).

R_{f}: 0.38 (4:1, hexane/ethyl acetate)

[α]_{D}=0.00

¹H NMR (CDCl₃, 400 MHz) δ_{H} ppm: 0.17 (6H, d, J=4.56 Hz, Si(CH₃)₂), 1.00 (9H, s, C(CH₃)₃), 2.40-2.56 (3H, m, CH₂), 2.88-2.98 (2H, m, 1×CHCHO, 1×CH₂), 3.70 (3H, s, OMe,), 3.85 (3H, s, OMe), 3.94 (3H, s, OMe), 3.95 (3H, s, OMe), 6.39 (1H, s, ArH), 6.46 (1H, d, J=7.32 Hz, C═CH), 6.81-6.85 (2H, m, ArH), 6.89-6.94 (1H, m, ArH)

¹³C NMR (CDCl₃, 400 MHz) δ_{c} ppm: −4.53 (Si(CH₃)₂), 18.47 (C(CH₃)₃), 22.69 (CH₂), 25.73 (C(CH₃)₃), 36.77 (CH₂), 49.81 (CHCHO), 55.50 (OMe), 55.91 (OMe), 60.89 (OMe), 61.55 (OMe), 109.00 (ArCH), 111.61 (ArCH), 120.76 (ArCH), 121.26 (C═CH) 121.59 (ArCH), 127.92 (ArC), 134.12 (ArC), 135.12 (ArC), 141.66 (ArC), 143.79 (ArC), 144.67 (ArC), 150.84 (ArC), 150.99 (ArC), 151.42 (ArC), 202.16 (CHO)

vₘₐₓ (DCM)/cm⁻¹:

HRMS: calculated 499.2516, found 499.2520, molecular formula (C₂₈H₃₉O₆Si).

### Aldehyde Oxidation: Synthesis of (7R)-9-{3-[(tert-butyldimethylsilyl)oxy]-4-methoxyphenyl}-2,3,4-trimethoxy-6,7-dihydro-5H-benzo[7]annulene-7-carboxylic acid 37.14

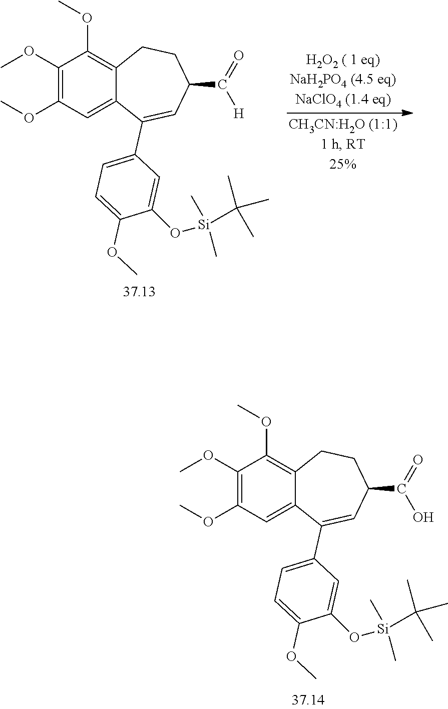 [SMILES: COc1ccc(C2=C[C@H](C=O)CCc3c2cc(OC)c(OC)c3OC)cc1O[Si](C)(C)C(C)(C)C ; COc1ccc(C2=C[C@H](C(=O)O)CCc3c2cc(OC)c(OC)c3OC)cc1O[Si](C)(C)C(C)(C)C]

To a stirred solution of 37.13 (0.08 g, 0.16 mmol) in acetonitrile and water (1:1, 1 mL) was added hydrogen peroxide 30% solution in water (0.02 mL, 0.17 mmol) and NaH₂PO₄ (0.1 g, 0.7 mmol). Sodium chlorite (0.03 g, 0.22 mmol) in water (0.5 mL) was then added dropwise to the reaction. After 1 hour the reaction was quenched by the addition of 2M HCl aqueous solution (5 mL). The reaction was extracted with diethyl ether (3×10 mL). The organic fractions were combined, dried over MgSO₄, filtered and concentrated under reduced pressure. The product was purified by flash column chromatography (stationary phase; silica gel 230-400 mesh, mobile phase; 5:95, methanol/ethyl acetate) to yield 37.14 as a yellow resin (0.02 g, 25%).

R_{f}: 0.14 (3:1, hexane/ethyl acetate)

[α]_{D}²⁵=+00.04

¹H NMR (CDCl₃, 400 MHz) δ_{H} ppm: 0.17 (6H, d, J=4.80 Hz, Si(CH₃)₂), 0.99 (9H, s, C(CH₃)₃), 2.20-2.42 (2H, m, CH₂), 2.38-2.57 (1H, m, CH₂), 2.92-3.03 (1H, m, CHCO₂H), 3.11-3.15 (1H, m, CH₂), 3.70 (3H, s, OMe,), 3.84 (3H, s, OMe), 3.94 (6H, s, OMe), 6.38 (1H, s, ArH), 6.47 (1H, d, J=6.58 Hz, C═CH), 6.78-6.82 (2H, m, ArH), 6.87-6.92 (1H, m, ArH)

¹³C NMR (CDCl₃, 400 MHz) δ_{c} ppm: −5.00 (Si(CH₃)₂), 18.00 (C(CH₃)₃), 22.39 (CH₂), 25.28 (C(CH₃)₃), 39.00 (CH₂), 42.31 (CHCO₂H), 55.04 (OMe), 55.62 (OMe), 60.44 (OMe), 61.16 (OMe), 108.33 (ArCH), 111.15 (ArCH), 120.33 (ArCH), 121.04 (ArCH), 122.72 (C═CH), 126.79 (ArC), 133.65 (ArC), 134.98 (ArC), 140.98 (ArC), 141.48 (ArC), 144.11 (ArC), 150.20 (ArC), 150.43 (ArC), 150.85 (ArC), 179.67 (CO₂H)

vₘₐₓ (DCM)/cm⁻¹:

HRMS: calculated 515.2465, found 515.2490, molecular formula (C₂₈H₃₉O₇Si).

### PFP Ester Formation: Synthesis of (7R)-9-{3-[(tert-butyldimethylsilyl)oxy]-4-methoxyphenyl}-2,3,4-trimethoxy-6,7-dihydro-5H-benzo[7]annulene-7-carboxylate 37.15

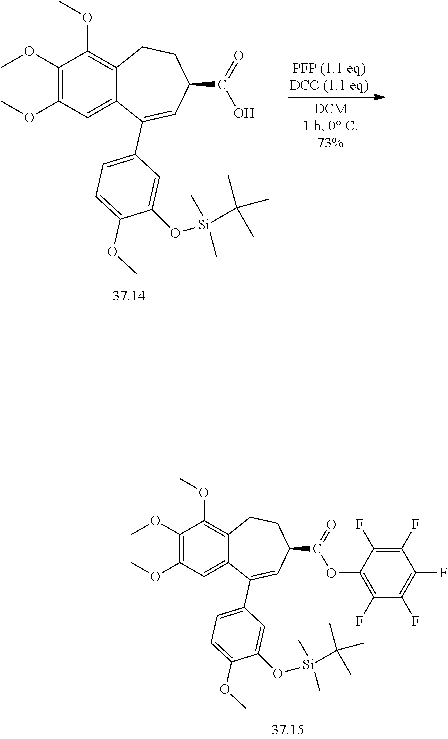

To a stirred solution of 37.14 (0.02 g, 0.04 mmol) and pentafluorophenol (0.01 g, 0.04 mmol) in dry DCM (0.5 mL) under an atmosphere of nitrogen at 0° C. was added a solution of DCC (0.01 g, 0.04 mmol) in dry DCM (0.5 mL). After 1 hour the reaction was quenched by the addition of water (5 mL) and the reaction extracted with diethyl ether (3×10 mL). The organic fractions were combined, dried over MgSO₄ and concentrated in vacuo. The resulting residue was then purified by flash column chromatography (stationary phase; silica gel 230-400 mesh, mobile phase; 20:1, hexane/ethyl acetate) to yield 37.15 as a colourless residue (0.02 g, 73%).

R_{f}: 0.66 (3:1, hexane/ethyl acetate)

¹H NMR (CDCl₃, 400 MHz) δ_{H} ppm: 0.17 (6H, d, J=6.15 Hz, Si(CH₃)₂), 1.00 (9H, s, C(CH₃)₃), 2.30-3.40 (5H, m, 4×CH₂, 1×CHCO₂), 3.72 (3H, s, OMe,), 3.85 (3H, s, OMe), 3.96 (3H, s, OMe), 3.97 (3H, s, OMe), 6.42 (1H, s, ArH), 6.50 (1H, d, J=6.55 Hz, C═CH), 6.79-6.86 (2H, m, ArH), 6.90-6.92 (1H, m, ArH)

¹⁹F NMR (CDCl₃, 400 MHz) δ_{F} ppm: −162.95-−162.71 (2F, m, ArF), −158.54-−158.42 (1F, m, ArF), 153.44 (2F, d, J=19.24 Hz, ArF)

vₘₐₓ (DCM)/cm⁻¹:

HRMS: calculated 703.2126, found 703.2111, molecular formula (C₃₄H₃₇F₅NaO₇Si).

### Hydroxamic Acid Synthesis: Synthesis of (7R)-9-{3-[(tert-butyldimethylsilyl)oxy]-4-methoxyphenyl}-N-hydroxy-2,3,4-trimethoxy-6,7-dihydro-5H-benzo[7]annulene-7-carboxamide 37.16

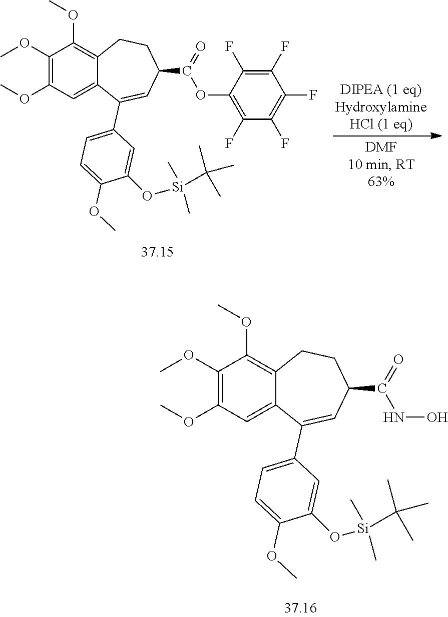 [SMILES: COc1ccc(C2=C[C@H](C(=O)Oc3c(F)c(F)c(F)c(F)c3F)CCc3c2cc(OC)c(OC)c3OC)cc1O[Si](C)(C)C(C)(C)C ; COc1ccc(C2=C[C@H](C(=O)NO)CCc3c2cc(OC)c(OC)c3OC)cc1O[Si](C)(C)C(C)(C)C]

To a stirred solution of 37.15 (0.02 g, 0.03 mmol) in DMF (0.5 mL) was added DIPEA (0.005 mL, 0.03 mmol) and hydroxylamine HCl (0.002 g, 0.03 mmol) at room temperature. After 10 minutes the reaction was quenched by the addition of 0.2M aqueous HCl solution (5 mL) and the reaction extracted with diethyl ether (3×10 mL). The organic fractions were combined, dried over MgSO₄ and concentrated in vacuo. The resulting residue was then purified by flash column chromatography (stationary phase; silica gel 230-400 mesh, mobile phase; 50:50:1, hexane/ethyl acetate/formic acid) to yield 37.16 as a clear residue (0.01 g, 63%).

R_{f}: 0.25 (50:50:1, hexane/ethyl acetate/formic acid)

¹H NMR (CDCl₃, 400 MHz) δ_{H} ppm: 0.15 (6H, d, J=4.90 Hz, Si(CH₃)₂), 0.99 (9H, s, C(CH₃)₃), 1.63-1.70 (1H, m, CH₂), 2.02-2.07 (3H, m, CH₂), 3.08-3.13 (1H, m, CHCONH), 3.71 (3H, s, OMe,), 3.84 (3H, s, OMe), 3.93 (3H, s, OMe), 3.94 (3H, s, OMe), 6.34-6.37 (2H, s, 1×ArH, 1×C═CH), 6.76-6.87 (3H, m, ArH)

vₘₐₓ (DCM)/cm⁻¹:

HRMS: calculated 530.2574, found 530.2562, molecular formula (C₂₈H₄₀NO₇Si).

### Phenol Deprotection: Synthesis of (R,Z)-6,7-dihydro-N-hydroxy-9-(3-hydroxy-4-methoxyphenyl)-2,3,4-trimethoxy-5H-benzo[7]annulene-7-carboxamide 37.17.

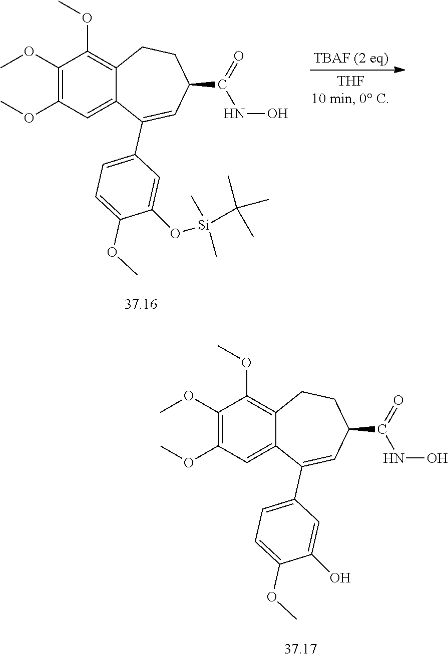 [SMILES: COc1ccc(C2=C[C@H](C(=O)NO)CCc3c2cc(OC)c(OC)c3OC)cc1O[Si](C)(C)C(C)(C)C ; COc1ccc(C2=C[C@H](C(=O)NO)CCc3c2cc(OC)c(OC)c3OC)cc1O]

To a stirred solution of 37.16 (0.01 g, 0.02 mmol) in dry THF (0.5 mL) under an atmosphere of nitrogen at 0° C. was added 1M TBAF solution (0.04 mL, 0.04 mmol) dropwise. After 10 minutes the reaction was quenched by the addition of water (5 mL) and the reaction extracted with diethyl ether (3×10 mL). The organic fractions were combined, dried over MgSO₄ and concentrated in vacuo. The resulting residue was then purified by flash column chromatography (stationary phase; silica gel 230-400 mesh, mobile phase; 50:50:1, hexane/ethyl acetate/formic acid) to yield 37.17 as a yellow residue (0.005 g, 60%).

R_{f}: 0.1 (50:50:1, hexane/ethyl acetate/formic acid)

¹H NMR (CDCl₃, 400 MHz) δ_{H} ppm: 2.31-2.43 (3H, m, CH₂), 3.09-3.12 (1H, m, CH₂), 3.34-3.39 (1H, m, CHCONH), 3.72 (3H, s, OMe,), 3.93 (3H, s, OMe,), 3.94 (3H, s, OMe,), 3.95 (3H, s, OMe,), 6.40-6.41 (2H, s, 1×ArH, 1×C═CH), 6.82-6.90 (3H, m, ArH)

vₘₐₓ (DCM)/cm⁻¹:

HRMS: calculated 438.1529, found 438.2223, molecular formula (C₂₂H₂₅NNaO₇).

Inversion of configuration of Z position to give R-alcohol.

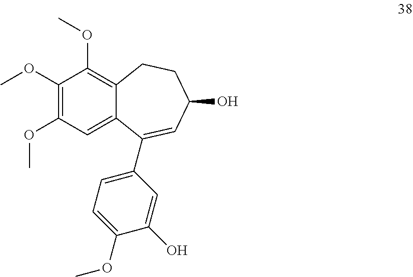 [SMILES: COc1ccc(C2=C[C@H](O)CCc3c2cc(OC)c(OC)c3OC)cc1O]

### Mesylate Formation and Displacement with Cesium Acetate: Synthesis of (7R)-6,7,8,9-tetrahydro-1,2,3-trimethoxy-5-(methoxymethoxy)-5H-benzo[7]annulen-7-yl acetate 38.2

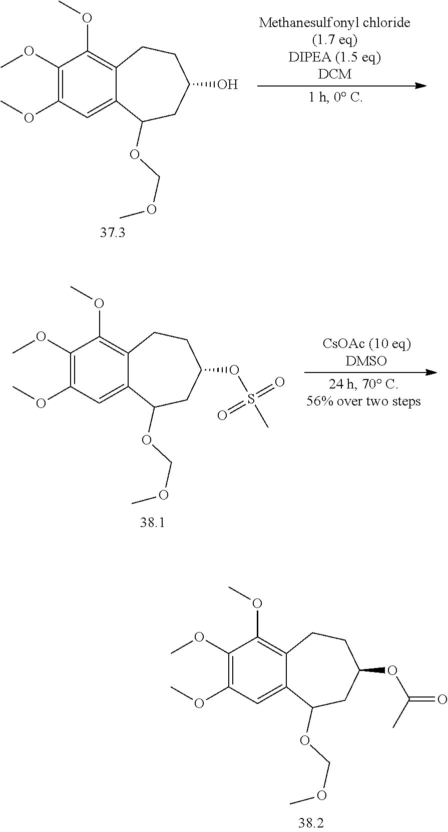 [SMILES: COCOC1C[C@@H](O)CCc2c1cc(OC)c(OC)c2OC ; COCOC1C[C@@H](OS(C)(=O)=O)CCc2c1cc(OC)c(OC)c2OC ; COCOC1C[C@H](OC(C)=O)CCc2c1cc(OC)c(OC)c2OC]

To stirred solution of 37.3 (0.11 g, 0.35 mmol) in dry DCM (1 mL) under an atmosphere of nitrogen at 0° C. was added methanesulfonyl chloride (0.05 mL, 0.60 mmol) followed by DIPEA (0.1 mL, 0.53 mmol). After 1 hour the reaction was quenched by the addition of water (5 mL) and the reaction extracted with diethyl ether (3×10 mL). The organic fractions were combined, dried over MgSO₄ and concentrated in vacuo. Cesium acetate was concurrently prepared as follows: To a stirred solution of cesium carbonate (0.57 g, 1.76 mmol) in dry methanol (10 mL) was added acetic acid (0.2 mL, 3.52 mmol). After 1 hour the methanol was removed under reduced pressure to yield cesium acetate as a white solid. The cesium acetate was added to a solution of the crude mesylate in DMSO (10 mL). The reaction was heated at 70° C. for 24 hours. The reaction was then quenched by the addition of water (50 mL) and extracted with diethyl ether (3×50 mL). The organic fractions were combined, dried over MgSO₄ and concentrated in vacuo. The resulting residue was then purified by flash column chromatography (stationary phase; silica gel 230-400 mesh, mobile phase; 4:1, hexane/ethyl acetate) to yield 38.2 as a colourless oil (0.07 g, 56%).

R_{f}: 0.46 (2:1, hexane/ethyl acetate)

¹H NMR (CDCl₃, 400 MHz) δ_{H} ppm: 1.27 (1H, m, major diastereomer, CH₂), 1.67-1.76 (2H, m, minor diastereomer CH₂), 1.89-2.00 (2H, m, major and minor diastereomers, CH₂), 2.05 (3H, s, minor diastereomer, OMe), 2.11 (3H, s, major diastereomer, OMe), 2.19-2.25 (3H, m, minor and major diastereomers, CH₂), 2.34-2.38 (1H, m, minor diastereomer, CH₂), 2.89-2.97 (2H, m, major diastereomer, CH₂), 3.29-3.34 (1H, m, minor diastereomer, CH₂), 3.40 (3H, s, major diastereomer, OMe), 3.45 (3H, s, minor diastereomer, OMe), 3.81 (3H, s, major diastereomer, OMe), 3.82 (3H, s, minor diastereomer, OMe), 3.87-3.89 (12H, m, major and minor diastereomers, 4×OMe), 4.61-4.66 (2H, m, major diastereomer, OCH₂OCH₃), 4.70-4.74 (2H, m, minor diastereomer, 1×OCH₂OCH₃, 1×CHOCO), 4.82 (1H, d, J=6.82 Hz, minor diastereomer, OCH₂OCH₃), 4.93 (1H, d, J=8.71 Hz, major diastereomer, CHOCH₂), 5.08-5.16 (1H, m, minor diastereomer, CHOCH₂), 5.35-5.40 (1H, m, major diastereomer, CHOCO), 6.71 (1H, s, major diastereomer, ArH), 6.92 (1H, s, minor diastereomer, ArH)

¹³C NMR (CDCl₃, 400 MHz) δ_{c} ppm: 19.00 (CH₂), 19.26 (CH₂), 21.37 (OCOCH₃), 21.46 (OCOCH₃), 32.54 (CH₂), 32.91 (CH₂), 38.67 (CH₂), 40.62 (CH₂), 55.45 (OMe), 55.77 (OMe), 56.00 (OMe), 56.04 (OMe), 60.85 (OMe), 61.37 (OMe), 71.36 (CHOCO), 71.73 (CHOCO), 73.67 (2×CHOCH₂), 94.16 (OCH₂O), 94.87 (OCH₂O), 103.64 (ArCH), 107.25 (ArCH), 124.54 (ArC), 126.68 (ArC), 136.79 (ArC), 137.66 (ArC), 140.89 (ArC), 141.45 (ArC), 150.98 (ArC), 150.99 (ArC), 151.36 (ArC), 151.56 (ArC), 170.20 (C═O), 170.25

vₘₐₓ (DCM)/cm⁻¹:

HRMS: calculated 377.1576, found 377.1565, molecular formula (C₁₈H₂₆NaO₇).

### Acetate Hydrolysis: Synthesis of (7R)-6,7,8,9-tetrahydro-1,2,3-trimethoxy-5-(methoxymethoxy)-5H-benzo[7]annulen-7-ol 38.3

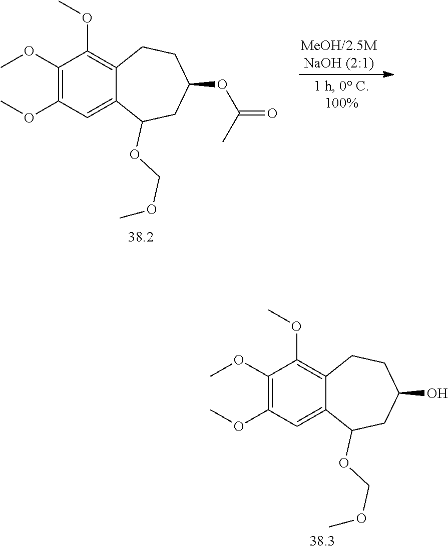 [SMILES: COCOC1C[C@H](OC(C)=O)CCc2c1cc(OC)c(OC)c2OC ; COCOC1C[C@H](O)CCc2c1cc(OC)c(OC)c2OC]

To a stirred solution of 38.2 (0.62 g, 1.75 mmol) in methanol (10 mL) at 0° C. was added 2.5 M NaOH aqueous solution (5 mL) dropwise. After 1 hour the methanol was removed under reduced pressure. The reaction was quenched by the addition of 2 M HCl aqueous solution (20 mL) and the reaction was extracted with diethyl ether (3×50 mL). The organic fractions were combined, dried over MgSO₄ and concentrated in vacuo. The resulting residue was then purified by flash column chromatography (stationary phase; silica gel 230-400 mesh, mobile phase; 1:1, hexane/ethyl acetate) to yield 38.3 as a colourless resin (0.55 g, 100%).

R_{f}: 0.12 (2:1, hexane/ethyl acetate)

¹H NMR (CDCl₃, 400 MHz) δ_{H} ppm: 1.62-2.27 (8H, m, minor and major diastereomers, CH₂), 2.62 (1H, s, br, minor diastereomer CH₂), 2.75-2.81 (1H, major diastereomer CH₂), 2.95-3.04 (1H, m, major diastereomer, CH₂), 3.15-3.21 (1H, m, minor diastereomer, CH₂), 3.40 (3H, s, major diastereomer, OMe), 3.42 (3H, s, minor diastereomer, OMe), 3.81 (3H, s, major diastereomer, OMe), 3.82 (3H, s, minor diastereomer, OMe), 3.86 (6H, s, minor and major diastereomers, 2×OMe), 3.88 (6H, s, major and minor diastereomers, 2×OMe), 4.10-4.16 (1H, m, minor diastereomer, CHOSi), 4.26-4.31 (1H, m, major diastereomer, CHOSi), 4.57-4.64 (4H, m, minor and major diastereomers, 2×OCH₂OCH₃), 4.74-4.78 (1H, m, minor diastereomer, CHOCH₂O), 4.88-4.94 (1H, m, major diastereomer, CHOCH₂O), 6.69 (1H, s, major diastereomer, ArH), 6.73 (1H, s, minor diastereomer, ArH)

¹³C NMR (CDCl₃, 400 MHz) δ_{c} ppm: 17.87 (CH₂), 18.28 (CH₂), 35.29 (CH₂), 35.91 (CH₂), 41.37 (CH₂), 55.02 (OMe), 55.21 (OMe), 55.56 (2×OMe), 60.39 (OMe), 60.92 (OMe), 68.46 (CHOSi), 71.36 (CHOSi), 93.57 (OCH₂OCH₃), 93.75 (OCH₂OCH₃), 107.00 (ArCH), 126.74 (ArC), 136.26 (ArC), 13.45 (ArC), 140.96 (ArC), 150.35 (ArC), 150.42 (ArC), 150.82 (ArC), 150.88 (ArC)

νₘₐₓ (DCM)/cm⁻¹:

HRMS: calculated 335.1471, found 335.1530, molecular formula (C₁₆H₂₄NaO₆).

### Silyl Protection: Synthesis of ((7R)-6,7,8,9-tetrahydro-1,2,3-trimethoxy-5-(methoxymethoxy)-5H-benzo[7]annulen-7-yloxy)(tert-butyl)diphenylsilane 38.4

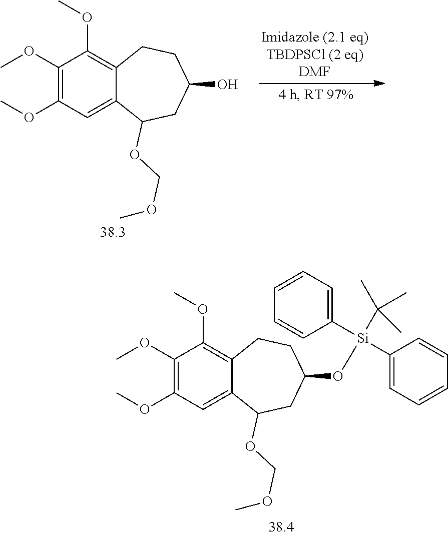 [SMILES: COCOC1C[C@H](O)CCc2c1cc(OC)c(OC)c2OC ; COCOC1C[C@H](O[Si](c2ccccc2)(c2ccccc2)C(C)(C)C)CCc2c1cc(OC)c(OC)c2OC]

To a stirred solution of 38.3 (0.55 g, 1.76 mmol) and imidazole (0.25 g, 3.52 mmol) in dry DMF (10 mL) under an atmosphere of nitrogen at room temperature was added tert-butyldiphenylsilyl chloride (0.92 mL, 3.52 mmol). After 4 hours the reaction was quenched by the addition of water 25 mL) and the reaction was extracted with diethyl ether (3×50 mL). The organic fractions were combined, dried over MgSO₄ and concentrated in vacuo. The resulting residue was then purified by flash column chromatography (stationary phase; silica gel 230-400 mesh, mobile phase; 20:1, hexane/ethyl acetate) to yield 38.4 as a colourless oil (0.94 g, 97%).

R_{f}: 0.40 (3:1, hexane/ethyl acetate)

¹H NMR (CDCl₃, 400 MHz) δ_{H} ppm: 1.07 (9H, s, minor diastereomer, C(CH₃)₃), 1.15 (9H, s, major diastereomer C(CH₃)₃), 1.22-1.37 (2H, m, minor and major diastereomers, CH₂), 1.65-1.73 (2H, m, minor and major diastereomers, CH₂), 1.93-2.00 (3H, m, minor and major diastereomers, CH₂), 2.16-2.20 (2H, m, minor diastereomer, CH₂), 2.82 (1H, s, major diastereomer, CH₂), 2.97-3.03 (1H, m, major diastereomer, CH₂), 3.14-3.19 (1H, q, J=7.20 Hz, minor diastereomer, CH₂), 3.24 (3H, s, minor diastereomer, OMe), 3.35 (3H, s, major diastereomer, OMe), 3.79 (3H, s, major diastereomer, OMe), 3.81 (3H, s, minor diastereomer, OMe), 3.86-3.90 (13H, m, minor and major diastereomers, 1×CHOSi, 4×OMe), 3.96-4.06 (1H, m, minor diastereomer, CHOSi), 4.31 (1H, s, br, major diastereomer, CHOCH₂), 4.42 (1H, d, J=10.62 Hz, minor diastereomer, CHOCH₂) 4.50 (H, d, J=6.77 Hz, minor diastereomer, CHOCH₂OCH₃), 4.54 (H, d, J=6.77 Hz, minor diastereomer, CHOCH₂OCH₃), 4.60 (2H, br s, major diastereomer, CHOCH₂OCH₃), 6.78 (1H, s, major diastereomer, ArH), 6.86 (1H, s, minor diastereomer, ArH), 7.39-7.48 (6H, m, minor and major diastereomers, ArH), 7.70-7.77 (4H, m, minor and major diastereomers, ArH).

¹³C NMR (CDCl₃, 400 MHz) δ_{c} ppm: 18.71 (C(CH₃)₃), 18.81 (C(CH₃)₃), 18.88 (2×CH₂), 19.35 (C(CH₃)₃), 26.48 (C(CH₃)₃), 26.61 (C(CH₃)₃), 35.93 (CH₂), 42.08 (CH₂), 44.25 (CH₂), 55.00 (OMe), 55.11 (OMe), 55.54 (OMe), 60.41 (OMe), 60.89 (OMe), 60.94 (OMe), 69.35 (CHOCH₂), 70.38 (CHOCH₂), 73.01 (CHOSi), 93.96 (OCH₂OCH₃), 102.62 (ArCH), 124.42 (ArC), 127.12 (2×ArCH), 129.18 (2×ArCH), 133.88 (ArC), 133.90 (ArC), 133.93 (ArC), 134.03 (ArC), 135.36 (2×ArCH), 135.40 (2×ArCH), 135.43 (2×ArCH), 137.59 (ArC), 140.21 (ArC), 150.37 (ArC), 150.41 (ArC), 150.62 (ArC), 150.93 (ArC).

νₘₐₓ (DCM)/cm⁻¹:

HRMS: calculated 573.2648, found 573.2645, molecular formula (C₃₂H₄₂NaO₆Si).

### MOM Removal and Oxidation: Synthesis of tert-butyldiphenyl{[(7R)-1,2,3-trimethoxy-5-(methoxymethoxy)-6,7,8,9-tetrahydro-5H-benzo[7]annulen-7-yl]oxy}silane 38.6

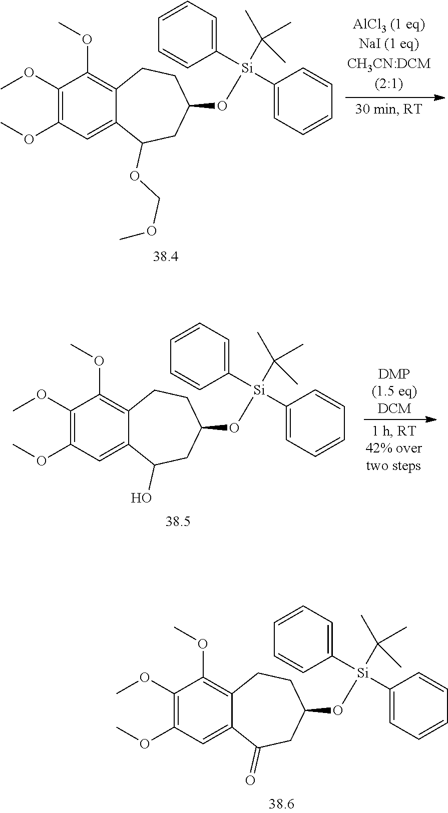 [SMILES: COCOC1C[C@H](O[Si](c2ccccc2)(c2ccccc2)C(C)(C)C)CCc2c1cc(OC)c(OC)c2OC ; COc1cc2c(c(OC)c1OC)CC[C@@H](O[Si](c1ccccc1)(c1ccccc1)C(C)(C)C)CC2O ; COc1cc2c(c(OC)c1OC)CC[C@@H](O[Si](c1ccccc1)(c1ccccc1)C(C)(C)C)CC2=O]

To a stirred solution of 38.4 (1.96 g, 3.47 mmol) in a mixture of CH₃CN and DCM (2:1, 24 mL) at room temperature was added aluminium chloride (0.46 g, 3.47 mmol) and sodium iodide (0.52 g, 3.47 mmol). After 30 minutes the reaction was quenched by the addition of saturated sodium bicarbonate aqueous solution (50 mL) and the reaction extracted with DCM (3×10 mL). To a stirred solution of the combined organic fractions was added Dess-Martin Periodinane (2.21 g, 5.21 mmol). After 1 hour the reaction was quenched by the addition of saturated sodium bicarbonate aqueous solution (50 mL) and the reaction was extracted with diethyl ether (3×50 mL). The organic fractions were combined, dried over MgSO₄ and concentrated in vacuo. The resulting residue was then purified by flash column chromatography (stationary phase; silica gel 230-400 mesh, mobile phase; 10:1, hexane/ethyl acetate) to yield 38.6 as a yellow oil (0.73 g, 42%).

R_{f}: 0.32 (3:1, hexane/ethyl acetate)

[α]_{D}²⁵=−55.56

¹H NMR (CDCl₃, 400 MHz) δ_{H} ppm: 1.06 (9H, s, C(CH₃)₃), 1.88-1.90 (1H, m, CH₂), 2.00-2.10 (H, m, CH₂), 2.92-2.98 (2H, m, CH₂), 3.04-3.10 (1H, m, CH₂), 3.10-3.19 (1H, m, CH₂), 3.86 (3H, s, OMe), 3.89 (3H, s, OMe), 3.95 (3H, s, OMe), 4.30-4.35 (1H, m, CHOSi), 7.17 (1H, s, ArH), 7.38-7.47 (6H, m, ArH), 7.64-7.77 (4H, m, ArH)

¹³C NMR (CDCl₃, 400 MHz) δ_{c} ppm: 18.70 (C(CH₃)₃), 21.47 (CH₂), 26.40 (C(CH₃)₃, 35.88 (CH₂), 49.84 (CH₂), 55.52 (OMe), 60.44 (OMe), 60.71 (OMe), 67.76 (CHOSi) 107.00 (ArCH), 127.20 (2×ArCH), 127.23 (2×ArCH), 129.28 (ArCH), 129.34 (ArCH), 130.44 (ArC), 133.27 (ArC), 133.46 (ArC), 134.17 (ArC), 135.31 (2×(ArCH), 135.36 (2×ArCH), 145.05 (ArC), 150.60 (ArC), 150.29 (ArC), 199.29 (C═O)

vₘₐₓ (DCM)/cm⁻¹:

HRMS: calculated 527.223, found 527.2244, molecular formula (C₃₀H₃₆NaO₅Si).

### Synthesis of Z-containing R—CH₂NH₂ Linker units 39/40

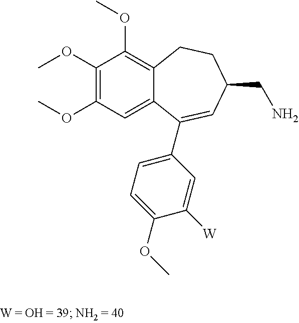

### Reduction of Nitrile: Synthesis of ((7R)-6,7,8,9-tetrahydro-1,2,3-trimethoxy-5-(methoxymethoxy)-5H-benzo[7]annulen-7-yl)methanamine 39.1

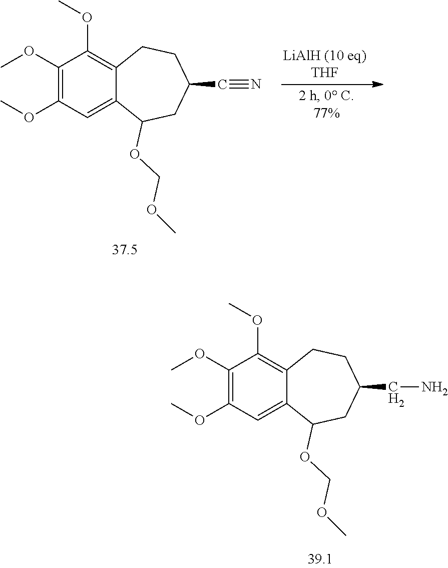 [SMILES: COCOC1C[C@H](C#N)CCc2c1cc(OC)c(OC)c2OC ; COCOC1C[C@H](CN)CCc2c1cc(OC)c(OC)c2OC]

To a stirred solution of LiAlH₄ (0.06 g, 1.56 mmol) under an atmosphere of nitrogen at 0° C. was added a solution of 37.5 (0.05 g, 0.16 mmol) dropwise. After 2 hours the reaction mixture was quenched by slowly adding to water (5 mL) at 0° C. The aqueous layer was then acidified by the addition of 2 M HCl aqueous solution (10 mL) and washed with diethyl ether (3×10 mL). The aqueous phase was then basified by the addition of 2.5 M NaOH aqueous solution (20 mL) and extracted with diethyl ether (3×50 mL). The organic fractions were combined, dried over MgSO₄ and concentrated in vacuo to yield 39.1 as a colourless oil (0.04 g, 77%).

R_{f}: 0.06 (3:1, ethyl acetate/methanol)

¹H NMR (CDCl₃, 400 MHz) δ_{H} ppm: 0.83-0.92 (1H, m, major diastereomer, CH₂), 1.07-1.16 (2H, m, major diastereomer, CH₂), 1.27-1.52 (2H, m, major and minor diastereomers, CH₂), 1.80-1.88 (1H, m, minor diastereomer, CHCH₂NH₂), 1.98-2.03 (3H, m, major and minor diastereomers, 2×CH₂, 1×CHCH₂NH₂), 2.13-2.31 (2H, major and minor diastereomers, CH₂), 2.58-2.62 (4H, m, major and minor diastereomers, 2×CH₂), 2.78-2.84 (1H, m, major diastereomer, CH₂), 3.06-3.12 (1H, q, J=7.55 Hz, major diastereomer, CH₂), 3.30-3.40 (1H, m, minor diastereomer, CH₂), 3.38 (3H, s, major diastereomer, OMe), 3.45 (3H, s, minor diastereomer, OMe), 3.80 (3H, s, major diastereomer, OMe), 3.81 (3H, s, minor diastereomer, OMe), 3.85 (3H, s, major diastereomer, OMe), 3.88 (9H, s, major and minor diastereomers, 3×OMe), 4.58 (2H, s, major diastereomer, OCH₂OCH₃), 4.71 (1H, d, J=6.80 Hz, minor diastereomer, OCH₂OCH₃), 4.76-4.83 (2H, m, major and minor diastereomers, 1×OCH₂OCH₃, 2×CHOCH₂), 6.59 (1H, s, major diastereomer, ArH), 6.92 (1H, s, minor diastereomer, ArH)

¹³C NMR (CDCl₃, 400 MHz) δ_{c} ppm: 22.59 (CH₂), 23.35 (CH₂), 31.21 (CH₂), 31.52 (CH₂), 36.71 (CH₂), 39.51 (CH₂), 39.60 (CHCH₂NH₂), 44.20 (CHCH₂NH₂), 49.00 (2×CH₂), 55.36 (OMe), 55.71 (OMe), 56.01 (OMe), 56.06 (OMe), 60.84 (OMe), 60.86 (OMe), 61.38 (2×OMe), 75.40 (CHOCH₂), 77.35 (CHOCH₂), 93.90 (OCH₂O), 95.00 (OCH₂O), 103.47 (ArCH). 109.27 (ArCH), 125.78 (ArC), 128.45 (ArC), 135.90 (ArC), 138.83 (ArC), 140.65 (ArC), 141.69 (ArC), 150.47 (ArC), 150.84 (ArC), 151.24 (ArC), 151.36 (ArC)

vₘₐₓ (DCM)/cm⁻¹:

HRMS: calculated 326.1967, found 326.1970, molecular formula (C₁₇H₂₈NO₅).

### BOC Protection of Amine: Synthesis of tert-butyl a7R)-6,7,8,9-tetrahydro-1,2,3-trimethoxy-5-(methoxymethoxy)-5H-benzo[7]annulen-7-yl)methylcarbamate 39.2

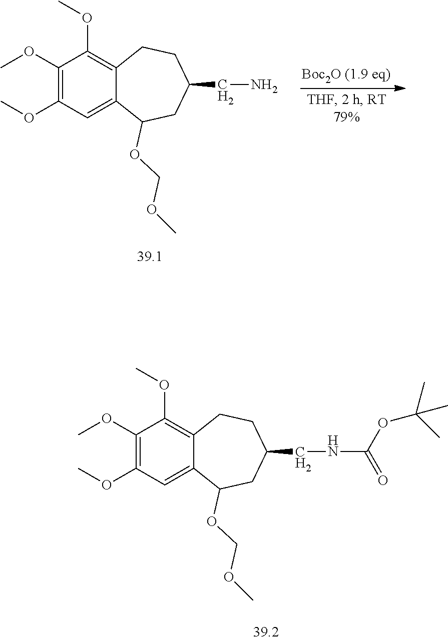 [SMILES: COCOC1C[C@H](CN)CCc2c1cc(OC)c(OC)c2OC ; COCOC1C[C@H](CNC(=O)OC(C)(C)C)CCc2c1cc(OC)c(OC)c2OC]

To a stirred solution of 39.1 (0.75 g, 2.30 mmol) in dry THF (3 mL) under an atmosphere of nitrogen at room temperature was added 1M Di-tert-butyl dicarbonate solution (4.41 mL, 4.41 mmol). After 2 hours the reaction was deemed to have gone to completion and the reaction was quenched by the addition of water (10 mL). The reaction was then extracted with diethyl ether (3×25 mL). The organic fractions were combined, dried over MgSO₄, filtered and concentrated under reduced pressure. The resulting residue was then purified by flash column chromatography (stationary phase; silica gel 230-400 mesh, mobile phase; 4:1, hexane/ethyl acetate) to yield 39.2 as a colourless oil (0.77 g, 79%).

R_{f}: 0.58 (1:1, hexane/ethyl acetate)

¹H NMR (CDCl₃, 400 MHz) δ_{H} ppm: 0.82-0.95 (1H, m, minor diastereomer, CH₂), 1.08-1.17 (1H, m, major diastereomer, CH₂), 1.31-1.34 (1H, d, J=12.1 Hz, minor diastereomer, CH₂), 1.45 (19H, s, major and minor diastereomers, 2×OC(CH₃)₃, 1×CH₂), 1.92-2.00 (3H, m, major and minor diastereomers, 1×CHCH₂NH, 2×CH₂), 2.07-2.28 (4H, m, major and minor diastereomers, 3×CH₂, 1×CHCH₂NH), 2.75-2.82 (1H, m, major diastereomer, CH₂), 2.99-3.11 (5H, m, major and minor diastereomers, 5×CH₂), 3.30-3.38 (1H, m, minor diastereomer, CH₂), 3.37 (3H, s, major diastereomer, OMe), 3.43 (3H, s, major diastereomer, OMe), 3.79 (3H, s, major diastereomer, OMe), 3.80 (3H, s, minor diastereomer, OMe), 3.84 (3H, s, major diastereomer, OMe), 3.87 (3H, s, minor diastereomer, OMe), 3.87 (3H, s, major diastereomer, OMe), 3.88 (3H, s, minor diastereomer, OMe), 4.56 (2H, s, major diastereomer, OCH₂OCH₃), 4.69 (1H, d, J=6.71 Hz, minor diastereomer, OCH₂OCH₃), 4.72-4.76 (2H, m, major and minor diastereomers, 2×CH₂), 4.79 (1H, d, J=6.71 Hz, minor diastereomer, OCH₂OCH₃), 6.57 (1H, s, major diastereomer, ArH), 6.91 (1H, s, minor diastereomer, ArH)

νₘₐₓ (DCM)/cm⁻¹:

HRMS: calculated 448.2311, found 448.2386, molecular formula (C₂₂H₃₅NNaO₇).

Removal of MOM and oxidation: Synthesis of tert-butyl aR)-6,7,8,9-tetrahydro-1,2,3-trimethoxy-5-oxo-5H-benzo[7]annulen-7-yl)methylcarbamate 39.4.

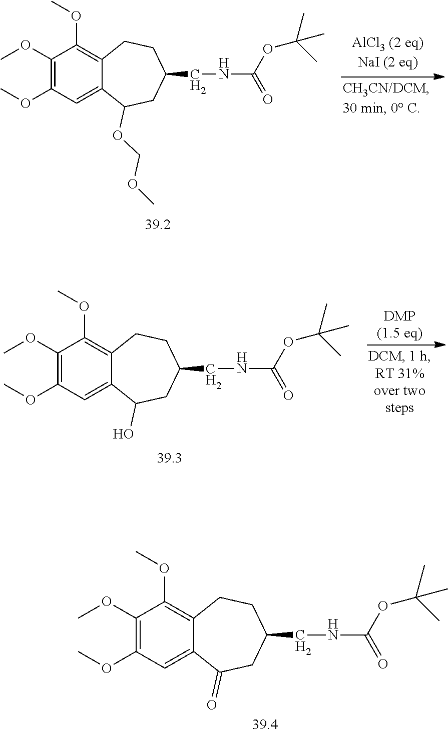

To a stirred solution of 39.2 (0.58 g, 1.28 mmol) in a mixture of acetonitrile and DCM (2:1, 24 mL) at 0° C. was added aluminium chloride (0.34 g, 2.56 mmol) and sodium iodide (3.8 g, 2.56 mmol). After 30 minutes the reaction was quenched by the addition of saturated sodium bicarbonate aqueous solution (50 mL). The reaction was extracted with DCM (3×10 mL). To a stirred solution of the combined organic fractions was added Dess-Martin Periodinane (0.82 g, 1.92 mmol). After 1 hour the reaction was quenched by the addition of saturated sodium bicarbonate aqueous solution (50 mL) and the reaction was extracted with diethyl ether (3×50 mL). The organic fractions were combined, dried over MgSO₄ and concentrated in vacuo. The resulting residue was then purified by flash column chromatography (stationary phase; silica gel 230-400 mesh, mobile phase; 10:1, hexane/ethyl acetate) to yield 39.4 as a yellow oil (0.15 g, 31%).

R_{f}: 0.17 (3:1, hexane/ethyl acetate)

[α]_{D}²⁵=−23.47

¹H NMR (CDCl₃, 400 MHz) δ_{H} ppm: 1.46 (10H, s, 1×C(CH₃)₃, 1×CH₂), 1.95-1.99 (1H, m, CH₂), 2.07-2.19 (1H, m, CHCH₂), 2.59-2.65 (1H, m, CH₂), 2.80-2.84 (2H, m, CH₂), 3.01-3.23 (3H, m, CH₂), 3.86 (3H, s, OMe), 3.90 (3H, s, OMe), 3.95 (3H, s, OMe), 7.15 (1H, s, ArH)

¹³C NMR (CDCl₃, 400 MHz) δ_{c} ppm: 21.92 (CH₂), 27.94 (C(CH₃)₃, 29.28 (CH₂), 33.80 (CHCH₂), 43.93 (CH₂), 44.50 (CH₂), 55.54 (OMe), 60.45 (OMe), 60.85 (OMe), 78.99 (OC(CH₃)₃), 106.94 (ArCH), 129.40 (ArC), 133.56 (ArC), 145.43 (ArC), 150.57 (ArC), 151.14 (ArC), 155.57 (C═O)

vₘₐₓ (DCM)/cm⁻¹:

HRMS: calculated 402.1893, found 402.1862, molecular formula (C₂₀H₂₉NNaO₆).

### Synthesis of Analogues where Position Y on the B-Ring is Functionalized

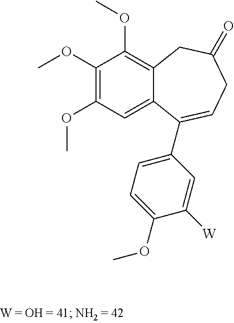

### Step 1: Synthesis of (2,3,4-Trimethoxyphenyl)methanol

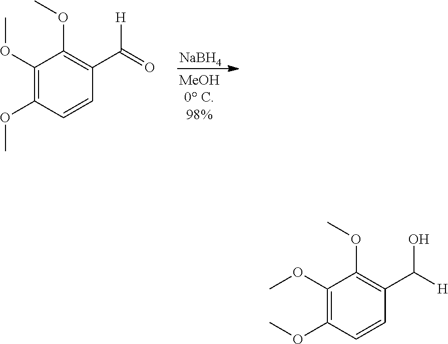

To a stirred solution of 2,3,4-trimethoxybenzaldehyde (10.50 g, 532 mmol) in methanol (150 mL) was added sodium borohydride (2.65 g, 70 mmol) at 0° C. The progress of the reaction was monitored by Thin Layer Chromatography (TLC) using hexane/ethyl acetate 4:1 as the mobile phase. After 30 min, the reaction was quenched by the addition of water (100 mL). The methanol was removed from the mixture in vacuo. The product was extracted with diethyl ether (1×150 mL, 2×75 mL). The organic extracts were combined, dried over MgSO₄ and concentrated to a yield the product as a colourless oil.

Yield: 10.25 g (51.71 mmol, 98%).

¹H NMR (CDCl₃, 400 MHz) δ_{H} ppm: 3.88 (s, 3H, CH₃), 3.90 (s, 3H, CH₃), 3.98 (s, 3H, CH₃), 4.64 (d, J=6.12 Hz, 2H, CH₂), 6.66 (d, J=8.44 Hz, 1H, ArH), 7.00 (d, J=8.44 Hz, 1H, ArH).

¹³C NMR (CDCl₃, 100.71 MHz) δ_{c} ppm: 55.5 (CH₃), 60.3 (COH₃), 60.7 (CH₃), 61.1 (CH₃), 122.9 (CH), 126.4 (CH), 141.4 (CH), 141.4 (Q), 151.2 (Q), 152.9 (Q).

### Synthesis of 1-(bromomethyl)-2,3,4-trimethoxybenzene

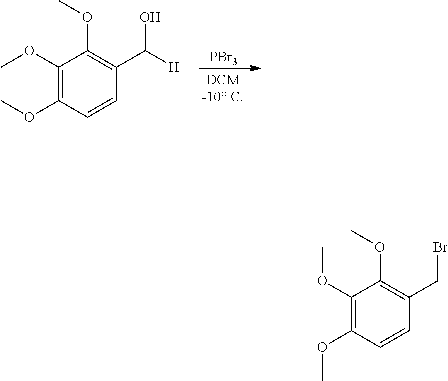

A solution of (2,3,4-Trimethoxyphenyl)methanol (5.00 g, 25.2 mmol) in dry DCM (35 mL) was stirred at −10° C. in an ice/NaCl bath. After 10 min, phosphorous tribromide (13.65 g, 4.75 mL, 50.5 mmol) was added dropwise by syringe. The reaction was monitored by TLC. After 90 min, the reaction was quenched with cool 10% sodium bicarbonate (50 mL) and washed with diethyl ether (75 mL). The ether extract was washed with 10% NaHCO₃ (3×50 mL). The ether extract was dried over MgSO₄ and concentrated in vacuo at a low temperature to prevent degradation of the product. The product was obtained as a colourless oil after being placed under high vacuum for not more than 2 h. The product was not purified and further and was used in directly in the following reaction.

¹H NMR (CDCl₃, 400 MHz) δ_{H} ppm: 3.88 (s, 3H, CH₃), 3.89 (s, 3H, CH₃), 4.04 (s, 3H, CH₃), 4.57 (s, 1H, CH₂Br), 6.66 (d, J=8.56 Hz, 1H, ArH), 7.07 (d, J=8.56 Hz, 1H, ArH).

### Synthesis of 2-(2,3,4-trimethoxyphenyl)acetonitrile

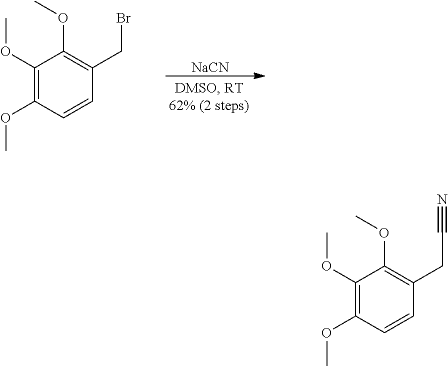

Sodium cyanide (2.75 g, 56.1 mmol) was stirred in DMSO (120 ml) at 25° C. for 30 min, ensuring maximum dissolution of the salt in the solvent. To this mixture was added 1-(bromomethyl)-2, 3, 4-trimethoxybenzene (4.90 g, 18.7 mmol). The mixture was left to stir for 2 h, by which time TLC (hexane/EtOAc 4:1) had shown that the reaction had proceeded to completion. The mixture was diluted with water (100 mL) and the product was extracted with ether (1×100 mL, 2×50 mL). The combined ether extracts were washed with water (100 mL), dried over MgSO₄ and concentrated in vacuo to produce a yellow oil. Column chromatography was used to isolate the product as a colourless solid using a hexane/EtOAc 9:1 mixture as the mobile phase.

Yield: 3.2 g (15.4 mmol, 62% over 2 steps).

¹H NMR (CDCl₃, 400 MHz) δ_{H} ppm: 3.66 (s, 2H, CH₂), 3.89 (s, 3H, CH₃), 3.90 (s, 3H, CH₃), 3.99 (s, 3H, CH₃), 7.03 (d, J=8.68 Hz, 1H, ArH), 7.03 (d, J=8.6 Hz, ArH).

¹³C NMR (CDCl₃, 100.71 MHz) δ_{c} ppm: 17.9 (CH₂), 55.6 (CH₃), 60.3 (CH₃), 60.4 (CH₃), 106.6 (ArCH), 115.6 (Q), 115.9 (Q), 123.0 (ArCH), 141.6 (Q), 150.9 (Q), 153.5 (Q).

MS (ESI): Calculated Mass 208.0968. Found 208.0286 (M+H⁺).

IR: 3621 w, 2941 s, 2250 m (Nitrile), 1603 s, 1496 s, 1420 s, 1260 s, 1097 s, 801 s, 688 s.

### Synthesis of 1-(2,3,4-Trimethoxyphenpl)pent-4-en-2-one

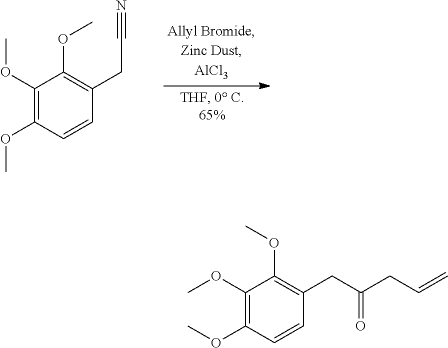 [SMILES: COc1ccc(CC#N)c(OC)c1OC ; C=CCC(=O)Cc1ccc(OC)c(OC)c1OC]

A 100 mL three necked round-bottomed flask was dried in an oven at 100° C. After 2 h, it was allowed to cool, before addition of 2-(2,3,4-trimethoxyphenyl)acetonitrile (3.00 g, 14.5 mmol) and zinc dust (3.78 g, 58 mmol). The flask was placed under vacuum and flushed periodically with nitrogen. Dry THF (45 mL) was added and the mixture was stirred on ice to which allyl bromide was added dropwise (2.63 g, 1.9 mL, 21.71 mmol). After 10 min on ice, AlCl₃ (0.77 g, 5.8 mmol) was added quickly. The mixture was stirred for 20 min at this temperature and stirred for a further hour at room temperature. TLC (hexane/EtOAc 4:1) indicated that the reaction had proceeded to completion. 1M HCl (75 mL) was added and stirred for 5 min to quench the reaction. The zinc solid was removed by decanting the mixture into a conical flask. The product was extracted by washing with ether (3×50 mL), drying the combined ether extracts over MgSO₄, and the mixture concentrated to give a dark yellow oil. The compound was isolated as a colourless oil following column chromatography using a 10:1 mixture of hexane/EtOAc as the mobile phase.

Yield: 2.35 g (9.4 mmol, 65%).

¹H NMR (CDCl₃, 400 MHz) δ_{H} ppm: 3.22 (d, J=7 Hz, 2H, CH₂), 3.65 (d, J=9.32 Hz, 2H, CH₂), 3.79 (s, 3H, CH₃), 3.83 (s, 3H, CH₃), 3.84 (s, 3H, CH₃), 5.08-5.17 (m, 2H, CH₂ alkene), 5.88-6.20 (m, 1H, alkene CH), 6.63 (d, J=3.24 Hz, 1H, ArH), 6.79 (d, J=8.52 Hz, 1H, ArH).

¹³C NMR (CDCl₃, 100.71 MHz) δ_{c} ppm: 43.2 (CH₂), 46.4 (CH₂), 55.4 (CH₃), 60.2 (CH₃), 60.2 (CH₃), 106.7 (CH), 118.2 (CH₂), 120.2 (Q), 124.6 (CH), 126.4 (Q), 130.2 (CH), 141.6 (Q), 151.4 (Q), 206.1 (C═O).

MS (ESI): Calculated 250.1278, Found 251.1279 (M+H⁺).

### Synthesis of 1-(2,3,4-Trimethoxyphenyl)pent-4-en-2-ol

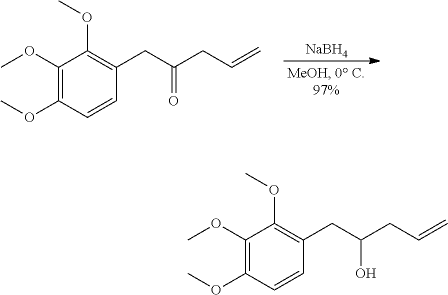 [SMILES: C=CCC(=O)Cc1ccc(OC)c(OC)c1OC ; "NaBH4" ; C=CCC(O)Cc1ccc(OC)c(OC)c1OC]

2-(2,3,4-Trimethoxyphenyl)acetonitrile (2.20 g, 8.8 mmol) was stirred at 0° C. in methanol (40 mL). Sodium borohydride (0.37 g, 9.68 mmol) was added slowly, and the reaction was stirred for 5 min before removal of the ice bath. TLC (hexane/EtOAc 4:1) showed completion of the reaction within 20 min. Water (40 mL) was added, and the methanol was removed by rotary evaporation. The aqueous solution was washed with ether (3×30 mL), the combined extracts were dried with MgSO₄ and concentrated in vacuo, leaving a colourless oil. This was purified via column (hexane/EtOAc 4:1) to give the product as a colourless oil.

Yield: 2.16 g (8.56 mmol, 97%).

¹H NMR (CDCl₃, 400 MHz) δ_{H} ppm: 2.24-2.35 (m, 2H, CH₂), 2.67-2.85 (m, 2H, CH₂), 3.84-3.93 (m, 1H, CHOH), 3.85 (s, 3H, CH₃), 3.87 (s, 3H, CH₃), 3.89 (s, 3H, CH₃), 5.14-5.19 (m, 2H, Alkene CH₂), 5.85-5.94 (m, 1H, alkene CH), 6.65 (d, J=8.44 Hz, 1H, ArH), 6.88 (d, J=8.48 Hz, 1H, ArH).

¹³C NMR (CDCl₃, 100.71 MHz) δ_{c} ppm: 37.0 (CH₂), 41.0 (CH₂), 55.5 (CH₃), 60.3 (CH₃), 60.4 (CH₃), 71.0 (CH), 106.8 (CH), 117.2 (CH₂), 123.9 (CH), 124.7 (CH), 134.5 (Q), 141.7 (Q), 151.5 (Q), 152.2 (Q).

MS (ESI): Calculated Mass 252.1362. Found 251.1298 (M⁻).

### Synthesis of (1-(2,3,4-Trimethoxyphenyl)pent-4-en-2-yloxy) (tert-butyl)diphenylsilane

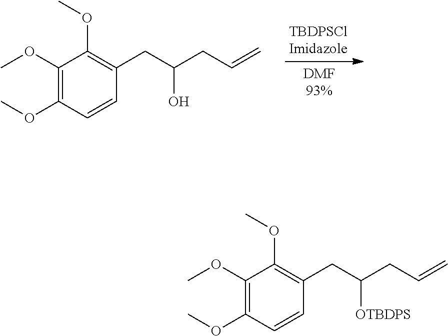

A round bottomed flask attached to a three necked adaptor was placed under vacuum and flushed with nitrogen sequentially three times. (2,3,4-Trimethoxyphenyl)pent-4-en-2-ol (2.45 g, 9.7 mmol) was placed into this flask under nitrogen with Imidazole (1.65 g, 24.2 mmol). Dry DMF (10 mL) was added via syringe to the flask and the mixture was stirred for ten min. Tert-butyl diphenylsilylchloride (2.9 mL, 11.1 mmol) was added and the reaction proceeded for 12 h. TLC (4:1 hexane/EtOAc) showed that the reaction had proceeded to completion. Brine (90 mL) was added to quench the reaction. The product was extracted with ether (2×90 mL then 2×50 mL). The combined ether extracts were washed with water (100 mL), dried over MgSO₄ and dried in vacuo leaving a colourless oil which was purified via column chromatography using a 50:1 mixture of hexane/EtOAc.

Yield: 4.12 g (9.02 mmol, 93%)

¹H NMR (CDCl₃, 400 MHz) δ_{H} ppm: 1.04 (s, 9H, ^{t}Bu), 2.07-2.12 (m, 2H, CH₂), 2.68-2.8 (m, 2H, CH₂), 3.66 (s, 3H, OCH₃), 3.83 (s, 3H, OCH₃), 3.85 (s, 3H, OCH₃), 4.05-4.08 (m, 1H, CHOSi), 4.90-5.02 (m, 2H, CH₂ alkene), 5.80-5.85 (m, 1H, CH alkene), 6.54 (d, J=8.48 Hz, 1H, ArH), 6.70 (d, J=8.48 Hz, 1H, ArH), 7.29-7.37 (m, 6H, ArH), 7.39-7.44 (m, 2H, ArH), 7.54-7.57 (m, 2H, ArH).

¹³C NMR (CDCl₃, 100.71 MHz) δ_{c} ppm: 19.4 (Q), 27.1 (CH₃), 37.4 (CH₂), 40.8 (CH₂), 56.0 (CH₃), 60.6 (CH₃), 60.7 (CH₃), 73.4 (CH), 106.9 (CH), 117.0 (CH₂), 125.0 (Q), 125.6 (CH), 127.4 (CH), 129.4 (CH), 134.4 (Q), 134.6 (Q), 135.1 (CH), 136.1 (CH), 152.2 (Q), 152.3 (Q).

MS (ESI): Calculated Mass 490.7058. Found 513.2427 (M+Na)⁺.

### Synthesis of 4-((tert-butyldiphenylsilyl)oxy)-5-(2,3,4-trimethoxyphenyl)pentan-1-ol

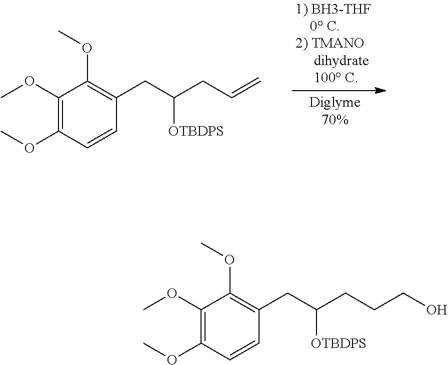

(1-(2,3,4-Trimethoxyphenyl)pent-4-en-2-yloxy)(tert-butyl)diphenylsilane (3.00 g, 6.1 mmol) was placed in a 3 necked round flask and equipped with a nitrogen balloon. 2-methoxyethyl ether (70 mL) was added via syringe and the mixture was stirred in an ice bath for 10 min. To this solution, a 1M solution of Borane in THF (18.3 mL, 18.3 mmol) was added dropwise. After 10 min the ice bath was removed. A further 50 min passed, when the THF was removed by rotary evaporation. Trimethylamine N-oxide dihydrate (4.74 g, 42.7 mmol) was then added to the flask, which was equipped with a reflux condenser. The mixture was heated under reflux for 2 h, by which time the reaction had completed. The reaction mixture was diluted with ethyl acetate (100 mL) and washed with warm water (100 mL). The organic layer was washed a further 6 times with water (100 mL), dried over MgSO₄ and concentrated in vacuo to give a yellow oil. This oil was purified using 3:1 hexane/EtOAc as a mobile phase for the resulting column. This yielded a colourless mobile oil.

Yield: 2.17 g (4.26 mmol, 70%).

¹H NMR (CDCl₃, 400 MHz) δ_{H} ppm: 1.57-1.65 (m, 4H, 2×CH₂), 2.71-2.78 (m, 2H, CH₂), 3.58-3.6 (m, 2H, CH₂), 3.68 (s, 3H, CH₃), 3.82 (s, 3H, CH₃), 3.84 (s, 3H, CH₃), 4.02-4.06 (m, 1H, CHOSi), 6.52 (d, J=8.52 Hz, 1H, ArH), 6.61 (d, J=8.44 Hz, 1H, ArH), 7.34-7.45 (m, 6H, ArH), 7.59-7.61 (m, 2H, ArH), 7.63-7.67 (m, 2H, ArH).

¹³C NMR (CDCl₃, 100.71 MHz) δ_{c} ppm: 13.7 (Q), 18.8 (Q), 26.6 (CH₃), 27.0 (CH₂), 28.6 (CH₂), 36.7 (CH₂), 55.4 (CH₃), 58.5 (CH₃), 60.1 (CH₃), 62.4 (CH₂), 71.4 (CH), 106.5 (CH), 124.3 (Q), 124.9 (CH), 126.9 (CH), 129.0 (CH), 133.8 (Q), 135.5 (CH), 141.6 (Q), 151.5 (Q).

MS (−ESI): Calculated Mass 522.2438. Found 507.1628 (M⁻).

### Synthesis of 4-((tert-butyldiphenylsilyl)-5-(2,3,4-trimethoxyphenyl)pentanoic acid

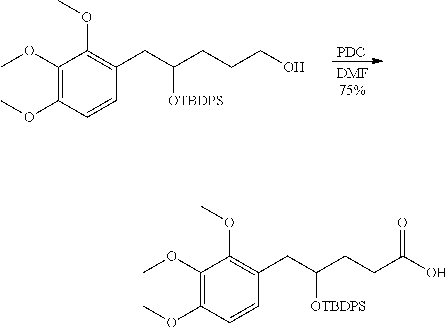

4-((tert-butyldiphenylsilyl)oxy)-5-(2,3,4-trimethoxyphenyl)pentan-1-ol (1.6 g, 3.1 mmol) was dissolved in DMF (2.5 mL). This was added dropwise to a suspension of pyridinium dichromate (3.55 g, 9.45 mmol) in DMF (6.5 mL). The reaction was stirred for 2 hr at room temperature after which time TLC analysis showed that the starting material had been used up. The reaction was quenched using water (50 mL). The product was extracted with ether (3×30 mL). The organic extracts were washed with water (3×50 mL). Following the washings, it was dried with MgSO₄ and the solvent was removed in vacuo. The product was purified by column chromatography (3:1 hexane/ethyl acetate) and obtained as a colourless gel.

Yield: 1.18 g (2.33 mmol, 75%).

¹H NMR (CDCl₃, 400 MHz) δ_{H} ppm: 1.15 (s, 9H, ^{t}Bu), 1.69-1.81 (m, 2H, CH₂), 2.49-2.59 (m, 2H, CH₂), 2.77-2.80 (m, 2H, CH₂), 3.71 (s, 3H, CH₃), 3.86 (s, 3H, CH₃), 3.87 (s, 3H, CH₃), 4.08-4.11 (1H, m, CHOSi) 6.55 (d, J=8.52 Hz 1H, ArH), 6.60 (d, J=8.52 Hz, 1H, ArH), 7.41-7.49 (m, 6H), 7.73 (m, 2H), 7.80 (m, 2H, ArH).

¹³C NMR (CDCl₃, 100.71 MHz) δ_{c} ppm: 13.8 (Q), 18.8 (Q), 26.5 (CH₃), 28.9 (CH₂), 29.8 (CH₂), 36.7 (CH₂), 55.5 (CH₃), 60.0 (CH₃), 60.2 (CH₃), 72.3 (CH), 106.5 (CH), 123.7 (Q), 124.7 (CH), 127.0 (CH), 129.1 (CH), 133.5 (Q), 135.5 (CH), 141.6 (Q), 151.6 (Q), 151.9 (Q), 179.9, (C═O).

MS (−ESI): Calculated Mass 522.2438. Found 521.4470 (M⁻).

### Synthesis of 8-((tert-butyldiphenylsilyl)oxy)-1,2,3-trimethoxy-6,7,8,9-tetrahydro-5H-benzo[7]annulen-5-one

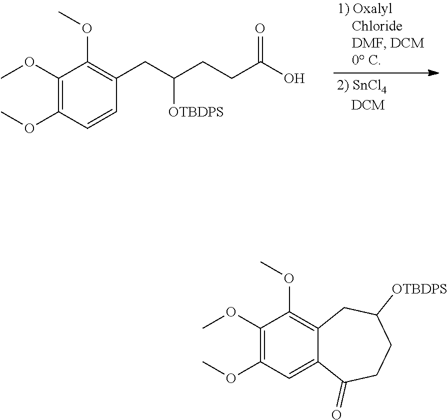

4-((tert-butyldiphenylsilyl)oxy)-5-(2,3,4-trimethoxyphenyl)pentanoic acid (0.27 g, 0.52 mmol) was dissolved in dry DCM (3 mL) and stirred on ice for 5 min under an atmosphere of N₂. Dry DMF (2 drops) was then added, followed by the dropwise addition of a 2M oxalyl chloride solution in DCM (0.34 mL, 0.77 mmol). After 30 min the ice bath was removed and the temperature was gradually allowed to rise to room temperature. After 45 min, the reaction vessel was heated gently for the next 45 min. TLC analysis showed that the carboxylic acid starting material had been used up. The solvents were removed under high vacuum and the vessel remained under vacuum for 3 hr to ensure dryness of the acyl chloride. After this period, DCM (10 mL) was added and the mixture was stirred in an ice/NaCl bath at −15° C. An SnCl₄ solution (1M in DCM) was then added dropwise (0.20 mL, 0.20 mmol). The mixture was stirred at this temperature for 1 h. Upon completion, brine (20 mL) was used to quench the reaction. The aqueous layer was washed with ether (3×20 mL). The organic extracts were dried over MgSO₄ and on the rotary evaporator at room temperature. This yielded a yellow oil which was purified by column chromatography to liberate small amounts of the desired compound.

Yield: 15 mg, 0.025 mmol, 6.2%.

¹H NMR (CDCl₃, 400 MHz) δ_{H}: 1.04 (s, 9H, ^{t}Bu), 2.71-2.79 (m, 2H, CH₂), 2.83-2.89 (m, 1H), 2.98-3.03 (m, 1H, CH₂), 3.72 (s, 3H, CH₃), 3.86-3.88 (m, 1H), 3.90 (s, 3H, CH₃), 3.94 (s, 3H, CH₃), 4.30-4.38 (m, 1H, CHOH), 7.37 (s, 1H, ArH), 7.37-7.47 (m, 6H, ArH), 7.63-7.68 (m, 4H, ArH).

¹³C NMR (CDCl₃, 100.71 MHz) δ_{c} ppm: 1.0 (Q), 19.1 (Q), 26.8 (CH₃), 32.1 (CH₂), 47.6 (CH₂), 56.0 (CH₃), 60.6 (CH₃), 60.9 (CH₃), 68.3 (CH), 104.6 (CH), 127.6 (CH), 127.7 (Q), 128.3 (CH), 133.5 (Q), 135.7 (CH), 147.5 (Q), 150.9 (Q), 152.1 (Q), 196.1 (Q).

MS (+ESI): Calculated Mass 504.2332. Found 505.2397 (M+H)⁺.

### Synthesis of Compound 43

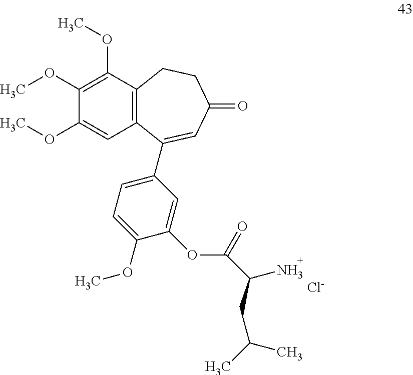 [SMILES: COc1ccc(C2=CC(=O)CCc3c2cc(OC)c(OC)c3OC)cc1OC(=O)[C@@H](N)CC(C)C ; [Cl-]]

### Step 1: Synthesis of 43.1-2,6-dichlorobenzoyl chloride coupling tBoc leucine to the phenol 1

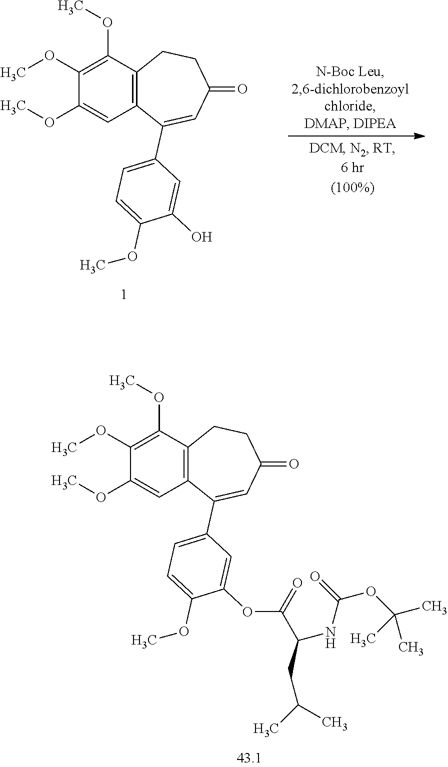 [SMILES: COc1ccc(C2=CC(=O)CCc3c2cc(OC)c(OC)c3OC)cc1O ; COc1ccc(C2=CC(=O)CCc3c2cc(OC)c(OC)c3OC)cc1OC(=O)[C@H](CC(C)C)NC(=O)OC(C)(C)C]

To a solution of tBoc-Leu (812 mg, 3.5 mmol), DIPEA (0.83 mL, 4.9 mmol) and 2,6-dichlorobenzoyl chloride (0.5 mL, 3.5 mmol) in dry DCM (3 mL) was added a solution of 1 (260 mg, 0.7 mmol) and DMAP (86 mg, 0.7 mmol) in dry DCM (2 mL) under an atmosphere of nitrogen at 0° C. The reaction was brought to room temperature and left stirring for 6 hr. After this time the reaction mixture was applied directly to a flash column and the product was isolated by flash column chromatography (stationary phase; silica gel 230-400 mesh, mobile phase; 6:1, hexane/ethyl acetate). All homogenous fractions were collected and the solvent was evaporated to afford the product 43.1 as a yellow solid (409 mg, 100%).

¹H NMR (CDCl₃) δ_{H} ppm: 0.95 (6H, dd, J=6.2 Hz, 22.5 Hz, 2×CH₃ (Leu)), 1.41 (9H, s, C(CH₃)₃), 1.64 (1H, m, CH₂ (Leu)), 1.74 (1H, m, CH₂ (Leu)), 1.82 (1H, m, CH (Leu)), 2.70 (2H, m, CH₂), 3.11 (2H, m, CH₂), 3.60 (3H, s, OCH₃), 3.83 (3H, s, OCH₃), 3.87 (3H, s, OCH₃), 3.92 (3H, s, OCH₃), 4.30 (1H, m, CHNH (Leu)), 6.35 (1H, d, J=14.5 Hz, C═CH), 6.41 (1H, d, J=12.6 Hz, ArH (A-ring)), 6.98 (2H, m, 2×ArH (C-ring)), 7.30 (1H, m, ArH (C-ring)).

¹³C NMR (CDCl₃) δ_{c} ppm: 20.2 (CH₂), 21.8 (CH₃ (Leu)), 22.9 (CH₃ (Leu)), 24.7 (CH(CH₃)₂ (Leu)), 28.4 (C(CH₃)₃ (Boc)), 41.6 (CH₂ (Leu)), 45.5 (CH₂), 52.2 (CHNH (Leu)), 55.9 (2×OCH₃), 60.9 (OCH₃), 61.4 (OCH₃), 79.9 (C(CH₃)₃ (Boc)), 111.4 (ArCH), 112.1 (ArCH), 123.9 (ArCH), 128.0 (ArCH), 128.2 (ArCH), 129.2 (ArC), 135.0 (ArC), 139.0 (ArC), 143.4 (ArC), 150.1 (ArC), 151.0 (ArC), 151.1 (ArC), 155.7 (ArC), 162.5 (ArC), 171.6 (NHC═O (Boc)), 177.1 (OC=0 (Leu)), 204.3 (C═O). νₘₐₓ/cm⁻¹ 2943.5, 1648.0, 1511.5, 1372.6, 1116.0

HRMS: calculated 583.67, found 606.2659 (+Na⁺), molecular formula (C₃₂H₄₁NO₉).

Melting point: 105° C.

### Step 2: Synthesis of 43—Removal of tBoc Group from Leu

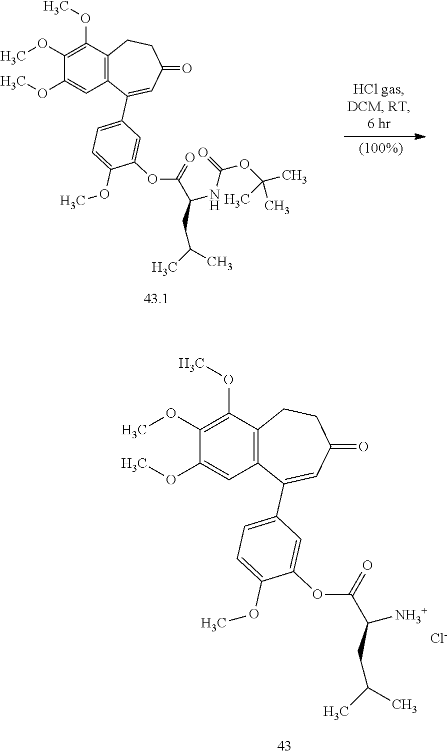

A solution of 43.1 (409 mg, 0.7 mmol) in DCM (1 mL) was acidified with HCl gas and stirred at room temperature for 20 min. After this time the solvent was removed in vacuo and the solid salt 43, a purple solid, was washed with hexane and dried under vacuum (340 mg, 100%).

¹H NMR (DMSO^{d6}) δ_{H} ppm: 0.88 (3H, dd, J=2.4 Hz, 6.4 Hz, CH₃ (Leu)), 0.93 (3H, d, J=6.0 Hz, CH₃ (Leu)), 1.60 (1H, m, CH₂ (Leu)), 1.74 (1H, m, CH₂ (Leu)), 1.82 (1H, m, CH (Leu)), 2.63 (2H, m, CH₂), 3.03 (2H, m, CH₂), 3.54 (3H, s, OCH₃), 3.78 (3H, s, OCH₃), 3.80 (3H, s, OCH₃), 3.83 (3H, s, OCH₃), 4.21 (1H, m, CHNH (Leu)), 6.29 (2H, d, J=6.7 Hz, C═CH, ArH (C-ring)).), 7.05 (1H, d, J=2.4 Hz, ArH (A-ring)), 7.25 (1H, d, J=8.0 Hz, ArH (C-ring)), 7.38 (1H, d, J=8.0 Hz, ArH (C-ring)).

¹³C NMR (DMSO^{d6}) δ_{c} ppm: 20.4 (CH₂), 22.5 (CH₃ (Leu)), 22.7 (CH₃ (Leu)), 24.2 (CH(CH₃)₂ (Leu)), 39.6 (CH₂ (Leu)), 46.0 (CH₂), 51.3 (CHNH), 56.2 (OCH₃), 56.6 (OCH₃), 61.0 (OCH₃), 61.7 (OCH₃), 111.7 (ArCH (B-ring)), 113.5 (ArCH (C-ring)), 123.8 (ArCH (A-ring)), 128.5 (ArCH (C-ring)), 129.3 (ArC), 131.9 (ArCH (C-ring)), 134.8 (ArC), 138.3 (ArC), 143.4 (ArC), 149.5 (ArC), 150.2 (ArC), 151.2 (ArC), 151.7 (ArC), 168.6 (ArC), 171.9 (C═O (Leu)), 203.1 (C═O).

νₘₐₓ/cm⁻¹ 2955.5, 1671.0, 1508.5, 1366.6, 1120.0

HRMS: calculated 483.56, found 484.2336 (+H⁺), molecular formula (C₂₇H₃₄NO₇).

### Synthesis of Hybrid 44

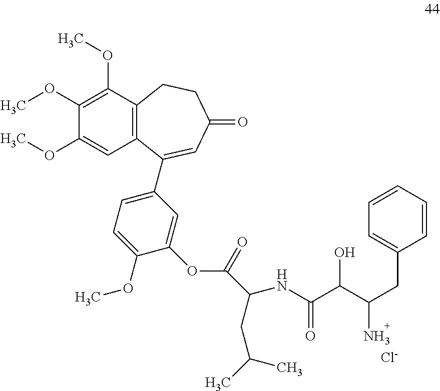 [SMILES: COc1ccc(C2=CC(=O)CCc3c2cc(OC)c(OC)c3OC)cc1OC(=O)C(CC(C)C)NC(=O)C(O)C(N)Cc1ccccc1 ; [Cl-]]

### Synthesis of 44.2—tBoc protection of AHPA

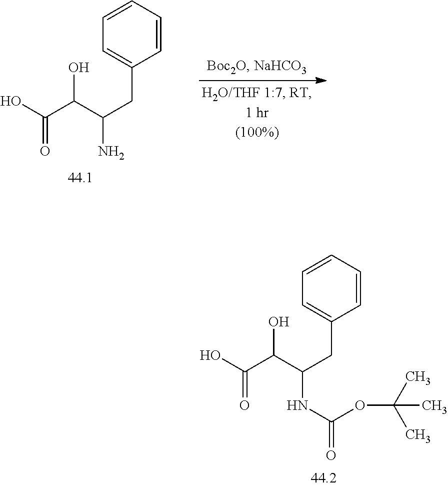 [SMILES: NC(Cc1ccccc1)C(O)C(=O)O ; CC(C)(C)OC(=O)NC(Cc1ccccc1)C(O)C(=O)O]

To a solution of AHPA 44.1 (800 mg, 4.1 mmol) and potassium carbonate (566 mg, 4.1 mmol) in water (2 mL) and THF (15 mL) was added a solution of ditertbutylcarbonate (894 mg, 4.1 mmol) in THF (15 mL) at 0° C. The temperature was brought to room temperature. After 1 hr, the organic solvent was removed in vacuo and the alkaline aqueous product was washed with diethyl ether (3×30 mL). The aqueous fraction was then acidified with 0.5 M HCl (30 mL) and the product was extracted into diethyl ether (3×30 mL). The organic layers were combined, dried over magnesium sulphate, filtered and concentrated to afford 44.2 as a white solid (1.2 g, 100%).

¹H NMR (CD₃OD) δ_{H} ppm: 1.35 (9H, s, C(CH₃)₃)), 2.91 (2H, dm, CH₂), 4.12 (H, d, J=2.0 Hz, CHOH), 4.27 (H, t, CHNH,), 7.28 (5H, m, 5×ArH (AHPA)).

¹³C NMR (CD₃OD) δ_{c} ppm: 27.7 (C(CH₃)₃), 38.0 (CH₂), 54.8 (CHNH), 70.4 (CHOH), 79.3 (C(CH₃)₃), 126.3 (ArCH), 128.3 (2×ArCH), 129.4 (2×ArCH), 138.0 (ArC), 156.2 (NHC═O), 175.2 (C═O).

νₘₐₓ/cm⁻¹ 3353.4, 2978.1, 1693.7, 1167.2

HRMS: calculated 295.34, found 294.1332 (−H⁺), molecular formula (C₁₅H₂₁NO₅).

### Synthesis of 44.3—The PFP Ester of N-Boc AHPA

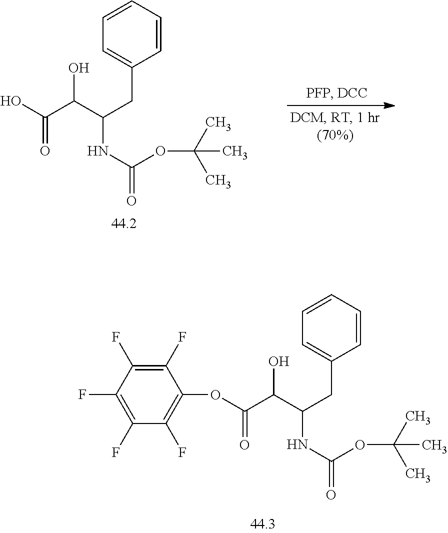 [SMILES: CC(C)(C)OC(=O)NC(Cc1ccccc1)C(O)C(=O)O ; CC(C)(C)OC(=O)NC(Cc1ccccc1)C(O)C(=O)Oc1c(F)c(F)c(F)c(F)c1F]

To a stirred solution of 44.2 (750 mg, 1.75 mmol) in anhydrous DCM/DMF (7 mL/3 mL) was added pentafluorophenol (380 mg, 2.1 mmol) followed by DCC (720 mg, 3.5 mmol) in dry DCM (5 mL) at 0° C. under an atmosphere of nitrogen. The temperature was allowed to increase to ambient over one hour. The reaction was then filtered to remove the by-product DCU and the product was purified by flash column chromatography (stationary phase; silica gel 230-400 mesh, mobile phase; 6:1, hexane/ethyl acetate). All homogenous fractions were combined and evaporated down to give 44.3 as a sticky opaque semi-solid (715 mg, 70%).

¹H NMR (CDCl₃) δ_{H} ppm: 1.44 (9H, s, C(CH₃)₃)), 3.08 (2H, m, CH₂), 4.47 (H, d, J=9.0 Hz, CHOH), 4.58 (H, s, CHNH,), 7.33 (5H, m, 5×ArH (AHPA)).

¹³C NMR (CDCl₃) δ_{c} ppm: 27.6 (C(CH₃)₃), 37.5 (CH₂), 54.7 (CHNH), 70.7 (CHOH), 80.1 (C(CH₃)₃), 126.3 (ArCH), 128.2 (2×ArCH), 128.9 (2×ArCH), 131.7 (ArC), 136.2 (ArC), 138.0 (ArC), 138.6 (ArC), 139.2 (ArC), 140.5 (ArC), 141.8 (ArC), 155.6 (NHC═O), 169.4 (C═O).

¹⁹F NMR (CDCl₃) δ_{F} ppm: −171.1, −165.5, −162.9, −158.2, −152.8.

νₘₐₓ/cm⁻¹ 3342.4, 2983.1, 1694.9, 1161.9

HRMS: calculated 461.38, found 484.1153 (+Na⁺), molecular formula (C₂₁H₂₀F₅O₅).

### Synthesis of 44.4—Coupling of N-Boc AHPA PFP 43 Ester to 44.3

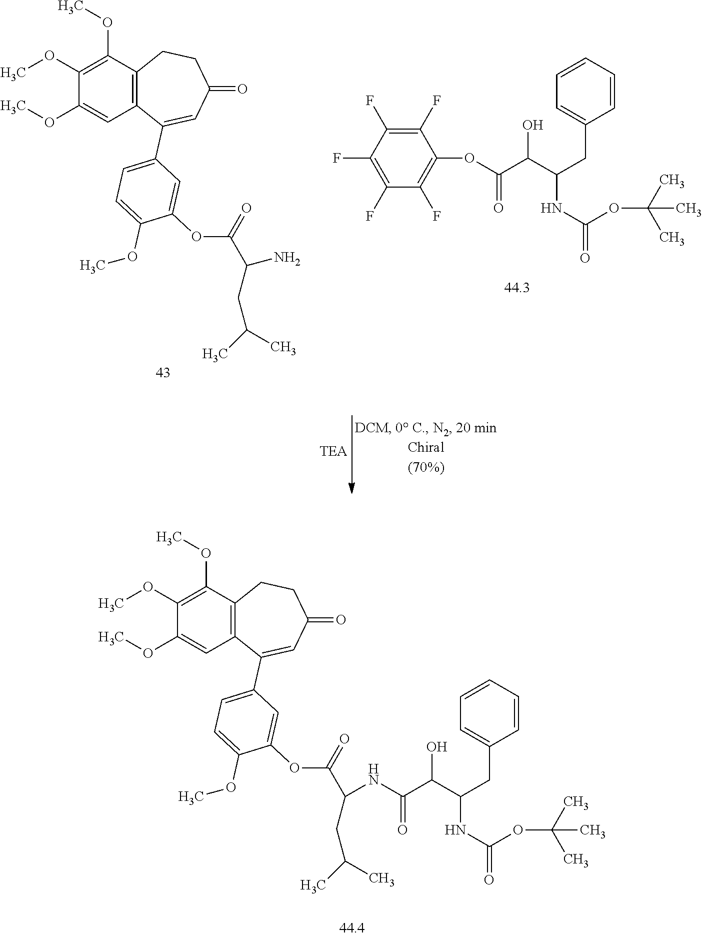 [SMILES: COc1ccc(C2=CC(=O)CCc3c2cc(OC)c(OC)c3OC)cc1OC(=O)C(N)CC(C)C ; CC(C)(C)OC(=O)NC(Cc1ccccc1)C(O)C(=O)Oc1c(F)c(F)c(F)c(F)c1F ; COc1ccc(C2=CC(=O)CCc3c2cc(OC)c(OC)c3OC)cc1OC(=O)C(CC(C)C)NC(=O)C(O)C(Cc1ccccc1)NC(=O)OC(C)(C)C]

To a solution of 43 (37 mg, 0.08 mmol) in anhydrous DCM (1 mL) was added a solution of 44.3 (70 mg, 0.15 mmol) in anhydrous DCM (1 mL) followed by Et₃N (0.02 mL, 0.15 mmol) under an atmosphere of nitrogen at 0° C. After 20 min the solvent volume was reduced under vacuum and loaded directly onto a flash column (stationary phase; silica gel 230-400 mesh, mobile phase; 6:1, hexane/ethyl acetate). All homogenous fractions were collected and the solvent was evaporated to afford the product as a pink solid (43 mg, 70%).

¹H NMR (CDCl₃) δ_{H} ppm: 1.01 (6H, dd, J=6.5 Hz, 8.2 Hz, 2×CH₃ (Leu)), 1.40 (9H, s, C(CH₃)₃ (Boc)), 1.75 (H, m, CH₂ (Leu)), 1.77 (1H, m, CH (Leu)), 1.88 (H, m, CH₂ (Leu)), 2.75 (2H, m, CH₂), 3.08 (2H, m, CH₂ (AHPA bzl)), 3.16 (2H, m, CH₂), 3.64 (3H, s, OCH₃), 3.87 (3H, s, OCH₃), 3.92 (3H, s, OCH₃), 3.96 (3H, s, OCH₃), 4.19 (1H, d, J=8.0 Hz, CHNH (AHPA)), 4.91 (1H, m, CHNH (Leu)), 5.09 (1H, d, J=9.6 Hz, CHOH (AHPA)), 6.38 (1H, d, J=11.5, C═CH), 6.97 (1H, s, ArH (A-ring)), 7.00 (1H, s, ArH (C-ring)), 7.02 (H, d, J=2.4 Hz, ArH (C-ring), 7.21-7.33 (5H, m, ArH (AHPA)), 7.30 (1H, s, ArH (C-ring)).

¹³C NMR (CDCl₃) δ_{c} ppm: 20.3 (CH₂), 21.6 (CH₃ (Leu)), 23.1 (CH₃ (Leu)), 24.7 (CH(CH₃)₂ (Leu)), 28.2 (C(CH₃)₃ (Boc)), 36.0 (CH₂ (AHPA bzl), 41.4 (CH₂ (Leu)), 45.7 (CH₂), 50.7 (CHNH (Leu)), 56.0 (OCH₃), 56.1 (OCH₃), 61.0 (OCH₃), 61.5 (OCH₃), 75.0 (CHNH (AHPA)), 72.5 (CHOH (AHPA)), 80.7 (C(CH₃)₃ (Boc)), 111.6 (ArCH), 112.2 (ArCH), 124.0 (ArCH), 126.7 (ArCH), 127.8 (ArCH (AHPA)), 128.4 (ArCH), 128.6 (2×ArCH (AHPA)), 129.1 (ArC), 129.3 (2×ArCH (AHPA)), 132.0 (ArC), 135.2 (ArC), 138.1 (ArC), 139.0 (ArC), 143.4 (ArC), 150.1 (ArC), 150.8 (ArC), 151.2 (ArC), 151.7 (ArC), 158.1 (NHC═O (Boc)), 170.6 (NHC═O (AHPA)), 172.8 (NHC═O (Leu)), 204.4 (C═O).

νₘₐₓ/cm⁻¹ 1742.5, 1266.3, 740.2, 700.4

HRMS: calculated 760.87, found 783.3594 (+Na⁺), molecular formula (C₄₂H₅₂N₂O₁₁).

### Synthesis of 44—Removal of tBoc Group 44.4

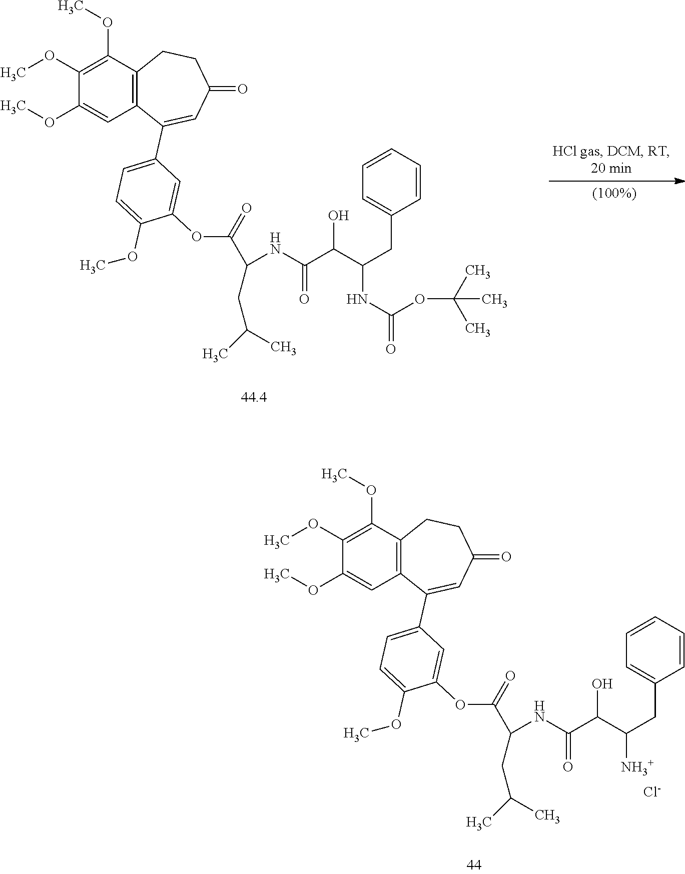

A solution of 44.4 (409 mg, 0.7 mmol) in DCM (1 mL) was acidified with HCl gas and stirred at room temperature for 20 min. Upon complete removal of the tBoc group the solvent was removed in vacuo to leave a dark pink solid (460 mg, 99%).

¹H NMR (DMSO^{d6}) δ_{H} ppm: 1.03 (6H, dd, J=5.0 Hz, 7.8 Hz, 2×CH₃ (Leu)), 1.84 (H, m, CH₂ (Leu)), 1.89 (1H, m, CH (Leu)), 1.93 (1H, m, CH₂ (Leu)), 2.66 (H, m, CH₂), 2.93 (H, dd, J=6.7 Hz, 13.1 Hz, CH₂ (AHPA bzl)), 3.12 (2H, m, d, J=5.9 Hz, CH₂), 3.18 (1H, d, J=7.4 Hz, CH₂ (AHPA bzl)), 3.56 (3H, s, OCH₃), 3.82 (3H, s, OCH₃), 3.84 (3H, s, OCH₃), 3.88 (3H, s, OCH₃), 4.18 (1H, s, CHNH (AHPA)), 4.69 (1H, m, CHNH (Leu)), 4.98 (1H, dd, J=8.0 Hz, CHOH (AHPA)), 6.33 (1H, d, J=4.0 Hz, C═CH), 6.36 (1H, s, ArH (A-ring)), 6.48 (1H, s, ArH (A-ring)), 6.99 (1H, s, ArH (C-ring)), 7.12 (1H, dd, J=8.3 Hz, 13.3 Hz, ArH (C-ring)), 7.23-7.39 (5H, m, 5×ArH (AHPA)).

¹³C NMR (DMSO^{d6}) δ_{c} ppm: 19.7 (CH₂), 20.9 (CH₃ (Leu)), 21.7 (CH₃ (Leu)), 24.6 (CH(CH₃)₂ (Leu)), 34.5 (CH₂ (AHPA bzl), 39.4 (CH₂ (Leu)), 45.0 (CH₂), 51.1 (CHNH (Leu)), 55.0 (OCH₃), 55.3 (OCH₃), 60.0 (OCH₃), 60.6 (OCH₃), 68.5 (CHNH (AHPA)), 97.5 (CHOH (AHPA)), 108.5 (ArCH (B-ring)), 111.7 (ArCH (A-ring)), 112.2 (ArCH (C-ring)), 123.5 (ArCH (C-ring)), 127.3 (ArCH (C-ring)), 127.7 (ArCH (AHPA)), 128.3 (2×ArCH (AHPA)), 128.6 (2×ArCH (AHPA)), 131.5 (ArC), 134.5 (ArC), 134.8 (ArC), 138.6 (ArC), 143.0 (ArC), 149.5 (ArC), 150.8 (ArC), 151.0 (ArC), 151.4 (ArC (AHPA)), 170.7 (ArC), 170.8 (NHC═O (AHPA)), 172.3 (NHC═O (Leu)), 204.6 (C═O).

νₘₐₓ/cm⁻¹ 2927.6, 1764.5, 1650.8, 1516.0, 1271.5, 1111.4, 1023.0, 702.7

HRMS: calculated 660.76, found 661.3698 (+H), molecular formula (C₃₇H₄₅N₂O₉).

### Synthesis of Compound 45

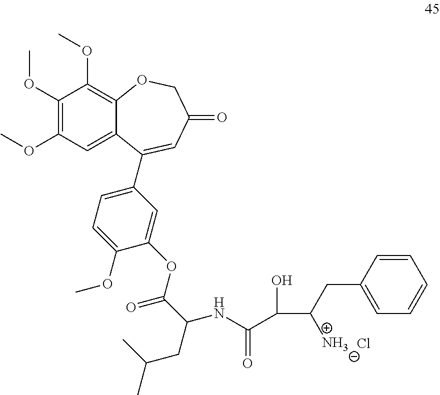 [SMILES: COc1ccc(C2=CC(=O)COc3c2cc(OC)c(OC)c3OC)cc1OC(=O)C(CC(C)C)NC(=O)C(O)C(N)Cc1ccccc1 ; Cl]

Synthesis of Intermediate 2-methoxy-5-(7,8,9-trimethoxy-3-oxo-2H-1-benzoxepin-5-yl)phenyl 2-{[(tert-butoxy)carbonyl]amino}-4-methylpentanoate 45.1.

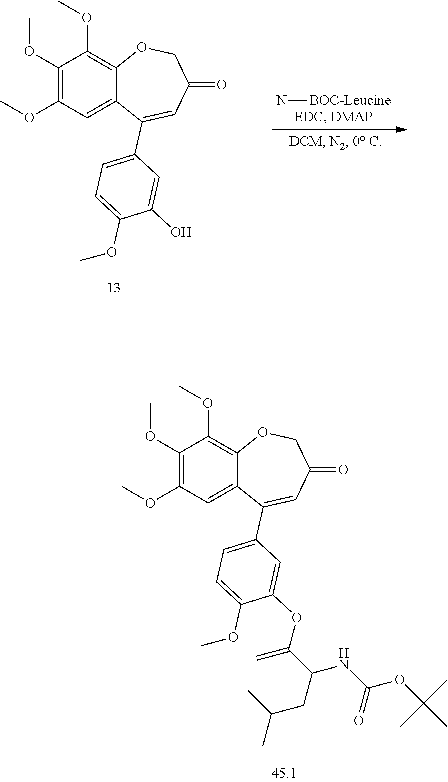

Phenol 5-(3-hydroxy-4-methoxyphenyl)-7,8,9-trimethoxy-2H-1-benzoxepin-3-one 13 (0.22 g, 0.6 mmoles) was dissolved in dry DCM (5 mL) and cooled to 0° C. under an atmosphere of nitrogen. To this was added sequentially N—BOC-Leucine (0.27 g, 0.12 mmoles) in dry DCM (5 mL), N-(3-dimethylaminopropyl)-N′-ethylcarbodiimide (0.23 g, 0.12 mmoles) in dry DCM (5 mL) and dimethylaminopyridine (4.3 mg, 0.35 mmoles). The reaction was then monitored by TLC and, when complete, quenched with water (20 mL), before extraction with diethyl ether (3×50 mL), being dried over MgSO₄, filtered and concentrated in vacuo. The resulting crude product was then purified by column chromatography (6:1, hexane:ethyl acetate) to give carbamate product 45.1 (0.224 g, 0.384 mmoles, 64%) as a brown oil.

¹H NMR (400 MHz, DMSO-d₆) δH: 1.03 (6H, d, 2×CH₃, J=6.02 Hz), 1.46 (9H, s, C(CH₃)₃), 1.67 (1H, m, CH(CH₃)₂), 1.87 (2H, m, Leu-CH₂), 3.65 (3H, s, OCH₃), 3.89 (3H, s, OCH₃), 3.99 (6H, s, 2×OCH₃), 4.53-4.60 (1H, m, CHCNH), 4.64 (2H, s, CH₂CO), 4.97 (1H, d, NH, J=8.53 Hz), 6.34 (1H, s, CH═C), 6.47 (1H, s, ArH), 7.01 (1H, d, ArH, J=8.53 Hz), 7.06 (1H, s, ArH), 7.31 (1H, d, ArH, J=8.53 Hz)

¹³C NMR (101 MHz, CHLOROFORM-d) δC: 22.9 (CH(CH₃)₂), 24.8 (CH(CH₃)₂), 28.3 (3×C(CH₃)₃), 41.6 (Leu-CH₂), 52.2 (CHNH), 55.9 (OCH₃), 56.1 (OCH₃), 61.3 (OCH₃), 61.9 (OCH₃), 79.9 (C(CH₃)₃), 81.2 (CH₂CO), 110.0 (ArH), 112.1 (CH═C), 124.2 (ArCH), 125.7 (ArC), 127.9 (ArCH), 128.4 (ArCH), 133.9 (ArC), 139.1 (ArC), 144.6 (ArC), 145.2 (ArC), 147.4 (ArC), 149.3 (ArC), 150.7 (ArC), 152.0 (C═CH), 155.4 (BOCOC═O), 171.6 (ArCOC═O), 200.48 (OCH₂C═O)

MS: calculated 585.2574, found 608.2556 (M+Na⁺).

### Synthesis of 2-methoxy-5-(7,8,9-trimethoxy-3-oxo-2H-1-benzoxepin-5-yl)phenyl 2-amino-4-methylpentanoate 45.2

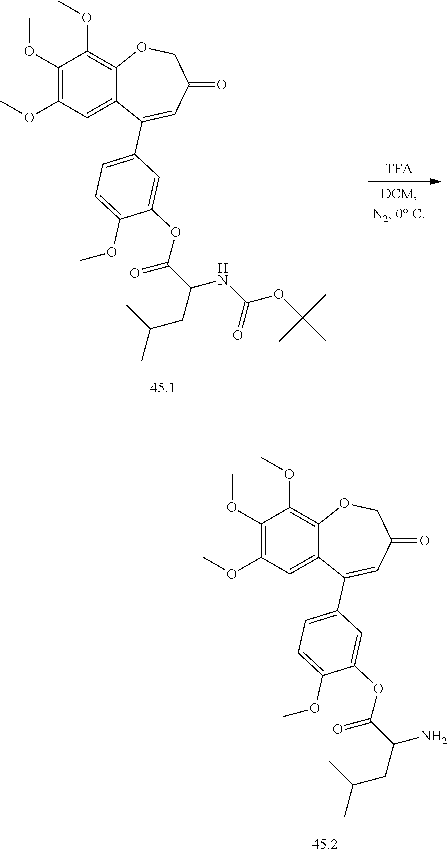

To carbamate 2-methoxy-5-(7,8,9-trimethoxy-3-oxo-2H-1-benzoxepin-5-yl)phenyl 2-{[(tert-butoxy)carbonyl]amino}-4-methylpentanoate 45.1 (0.19 g, 0.39 mmoles) under an atmosphere of nitrogen was added trifluoroacetic acid:DCM (1:1, 1 mL) at 0° C. After 5 min, the reaction was dried with nitrogen gas. The residue left was redissolved in ether (10 mL) and stirred, to which sodium hydrogencarbonate (1 mL, 5%) was added for 5 min. The organic layer was then separated and dried with MgSO₄, filtered, before being condensed under reduced pressure to afford free amine 45.2 (0.165 g, 0.304 mmoles, 87%) as a brown solid.

¹H NMR (400 MHz, CHLOROFORM-d) δH: 1.01 (6H, t, 2×CH₃, J=7.03 Hz), 1.55-1.65 (1H, m, CH(CH₃)₂), 1.76-1.99 (2H, m, Leu-CH₂), 2.21 (2H, br. s, NH₂), 3.66 (3H, s, OCH₃), 3.75-3.81 (1H, m, CHNH₂), 3.89 (3H, s, OCH₃), 3.99 (6H, s, 2×OCH₃), 4.65 (2H, s, CH₂C═O), 6.35 (1H, s, CH═C), 6.48 (1H, s, ArH), 6.99-7.05 (2H, m, 2×ArH), 7.28-7.32 (1H, m, ArH)

¹³C NMR (101 MHz, CHLOROFORM-d) δC: 21.4 (1×CH(CH₃)₂), 22.6 (1×CH(CH₃)₂), 24.3 (CH(CH₃)₂), 43.3 (Leu-CH2), 52.4 (CHNH₂), 55.5 (OCH₃), 55.7 (OCH₃), 60.9 (OCH₃), 61.5 (OCH₃), 80.7 (CH₂CO), 109.5 (ArCH), 111.6 (CH═C), 123.6 (ArCH), 125.3 (ArC), 127.4 (ArCH), 127.9 (ArCH), 133.5 (ArC), 138.8 (ArC), 144.1 (ArC), 144.8 (ArC), 146.9 (ArC), 148.8 (ArC), 150.3 (ArC), 151.6 (C═CH), 174.0 (ArCOC═O), 200.11 (OCH₂C═O)

MS: calculated 485.205, found 508.1924 (M+Na⁺).

### Synthesis of Intermediate 2-methoxy-5-(7,8,9-trimethoxy-3-oxo-2H-1-benzoxepin-5-yl)phenyl 2-(3-{[(tert-butoxy)carbonyl]amino}-2-hydroxy-4-phenylbutanamido)-4-methylpentanoate 45.3

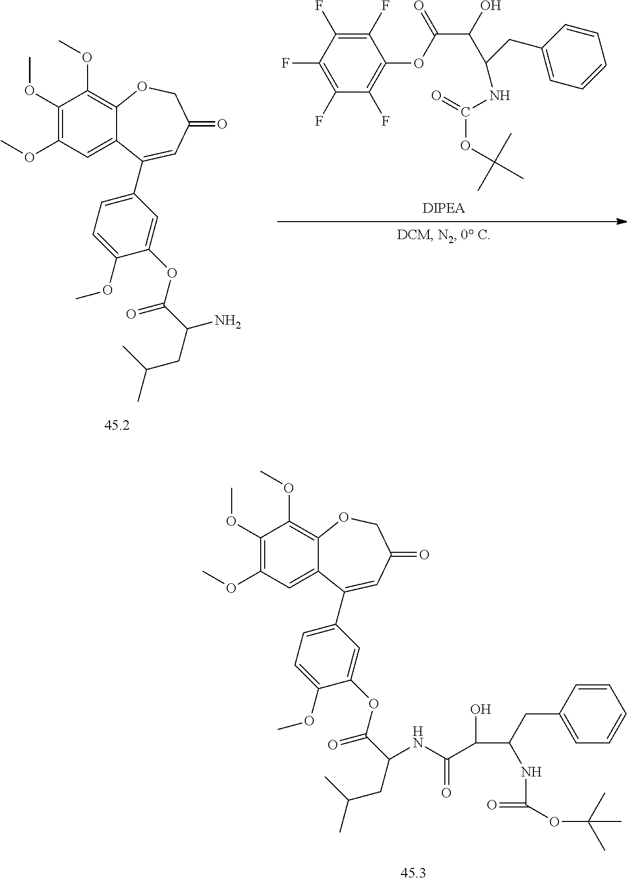 [SMILES: COc1ccc(C2=CC(=O)COc3c2cc(OC)c(OC)c3OC)cc1OC(=O)C(N)CC(C)C ; CC(C)(C)OC(=O)NC(Cc1ccccc1)C(O)C(=O)Oc1c(F)c(F)c(F)c(F)c1F ; COc1ccc(C2=CC(=O)COc3c2cc(OC)c(OC)c3OC)cc1OC(=O)C(CC(C)C)NC(=O)C(O)C(Cc1ccccc1)NC(=O)OC(C)(C)C]

Amine 45.2 (0.165 g, 0.34 mmoles), was dissolved in anhydrous DCM (5 mL) under an atmosphere of nitrogen, and cooled to 0° C. To this was added, in anhydrous DCM and under nitrogen, pentafluorophenyl ester pentafluorophenyl 3-{[(tert-butoxy) carbonyl]amino}-2-hydroxy-4-phenylbutanoate (0.14 g, 0.296 mmoles), followed by diisopropylethylamine (62 μl, 0.355 mmoles). The reaction was then monitored by TLC until such time as no further progress was observed. Solvent was then removed by blowing off with nitrogen gas and the remaining residue purified via column chromatography (1:1, hexane:ethyl acetate) to afford Boc-Bestatin compound 45.3 (90 mg, 0.11 mmoles, 35%) as a brown residue.

¹H NMR (400 MHz, CHLOROFORM-d) δH: 1.02 (6H, dd, CH(CH₃)₂), J=8.03, 6.53 Hz), 1.41 (9H, s, C(CH₃)₃), 1.72-1.96 (3H, m, 1×CH(CH₃)₂, 1×Leu-CH₂), 3.04-3.31 (2H, m, Ar—CH₂), 3.65 (3H, s, OCH₃), 3.89 (3H, s, OCH₃), 3.98 (1H, s, CHNH), 4.01 (6H, s, 2×OCH₃), 4.21 (1H, d, CHOH, J=2.76 Hz), 4.65 (2H, s, OCH₂), 4.86-4.94 (1H, m, Leu-CHNH), 5.01 (1H, d, NH, J=7.03 Hz), 5.74-5.92 (1H, br. s, OH), 6.33 (1H, s, ArH), 6.47 (1H, s, CH═C), 7.01 (1H, d, ArH, J=8.53 Hz), 7.06 (1H, d, ArH, J=2.01 Hz), 7.22-7.28 (3H, m, 3×ArH), 7.30-7.36 (3H, m, 3×ArH), 7.38 (1H, br. s., NH)

¹³C NMR (101 MHz, CHLOROFORM-d) δC: 21.6 (1×CH(CH₃)₂), 23.1 (1×CH(CH₃)₂), 24.8 (CH(CH₃)₂), 28.2 (C(CH3)3), 31.9 (ArC CH₂), 41.4 (Leu-CH₂), 50.4 (Leu-CHNH), 56.0 (OCH₃), 56.2 (OCH₃), 61.3 (OCH₃), 61.9 (OCH₃), 74.8 (COH), 80.7 (C(CH₃)₃), 81.1 (OCH₂), 110.1 (ArCH), 112.1 (ArCH), 124.1 (ArCH), 125.7 (ArC), 126.7 (CH═C), 128.0 (ArCH), 128.4 (ArCH), 128.6 (2×ArCH), 129.3 (2×ArCH), 134.0 (ArC), 138.0 (ArC), 139.1 (ArC), 144.7 (ArC), 145.3 (ArC), 147.5 (ArC), 149.3 (ArC), 150.8 (ArC), 152.0 (C═CH), 156.1 (BOC—C═O) 170.5 (ArOC═O), 172.7 (Leu-NC═O), 200.5 (CH₂C═O)

MS: calculated 762.3364, found 785.3245 (M+Na⁺).

### Synthesis of 2-methoxy-5-(7,8,9-trimethoxy-3-oxo-2H-1-benzoxepin-5-yl)phenyl 2-(3-amino-2-hydroxy-4-phenylbutanamido)-4-methylpentanoate 45

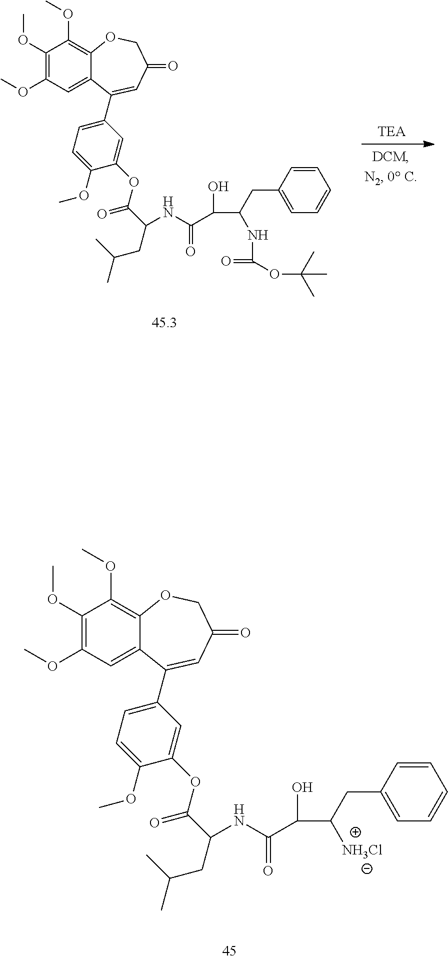

To carbamate compound 45.3 (0.9 g, 0.11 mmoles), under an atmosphere of nitrogen, was added trifluoroacetic acid in dry DCM (1:1, 1 mL). The reaction was stirred at 0° C. for 5 min, after which the solvent was blown off with nitrogen gas. The crude residue was then redissolved in diethyl ether (10 mL) and sodium hydrogencarbonate (0.5 mL, 5% aq. solution) added. This biphasic mixture was then allowed to stir for 5 min before the aqueous layer was removed. The remaining organic layer was then dried with MgSO₄, and concentrated. HCl gas was then blown through, prompting a yellow salt to crash out. This salt was then washed with diethyl ether (3×5 mL) which was decanted off, to leave amine salt 45 (50 mg, 0.076 mmoles, 69%) as a yellow solid.

¹H NMR (600 MHz, DMSO-d₆) δH: 0.92 (6H, dd, 2×CH₃, J=14.68, 5.65 Hz), 1.69-1.82 (3H, m, 1×CH(CH₃)₂, 1×Leu-CH₂), 2.78-2.93 (2H, m, ArC—CH₂), 3.48 (3H, s, OCH₃), 3.49-3.52 (1H, m, CHNH₃), 3.77 (3H, s, OCH₃), 3.82 (3H, s, OCH₃), 3.86 (3H, s, OCH₃), 3.99-4.02 (1H, m, CHOH), 4.48-4.54 (1H, m, Leu-CHNH), 4.65 (2H, s, OCH₂), 6.22 (1H, s, ArH), 6.29 (1H, s, CH═C), 6.67 (1H, br. S, OH), 6.98 (1H, s, ArH), 7.18 (1H, s, ArH), 7.19 (1H, s, ArH), 7.21-7.26 (3H, m, 3×ArH), 7.29-7.33 (2H, m, 2×ArH), 7.87 (2H, br. s, NH2), 8.53 (1H, s, NH)

¹³C NMR (151 MHz, DMSO-d) δC: 21.6 (1×CH(CH₃)₂), 22.6 (1×CH(CH₃)₂), 24.2 (CH(CH₃)₂), 34.6 (ArC—CH₂), 39.3 (Leu-CH₂), 50.6 (CHNH), 54.3 (CHNH₂), 55.8 (OCH₃), 56.0 (OCH₃), 60.8 (OCH₃), 61.4 (OCH₃), 68.5 (CHOH), 80.8 (OCH₂), 109.9 (ArCH), 112.9 (ArCH), 123.5 (ArCH), 125.2 (ArC), 126.9 (ArCH), 127.9 (1×ArCH, 1×CH═C), 128.6 (2×ArCH), 129.3 (2×ArCH), 133.1 (ArC), 136.1 (ArC), 138.5 (ArC), 144.2 (ArC), 145.0 (ArC), 147.0 (ArC), 148.8 (ArC), 149.7 (C═CH), 151.6 (ArC), 170.3 (OC═O), 171.3 (NHC═O), 199.6 (CH₂C═O)

MS: calculated 662.2839 (free amine), found 663.2937 (M+H⁺).

### Synthesis of Compound 46

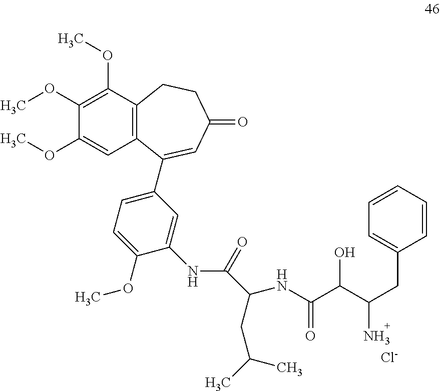 [SMILES: COc1ccc(C2=CC(=O)CCc3c2cc(OC)c(OC)c3OC)cc1NC(=O)C(CC(C)C)NC(=O)C(O)C(N)Cc1ccccc1 ; [Cl-]]

### Synthesis of 46.1—N-Boc Leu Coupling

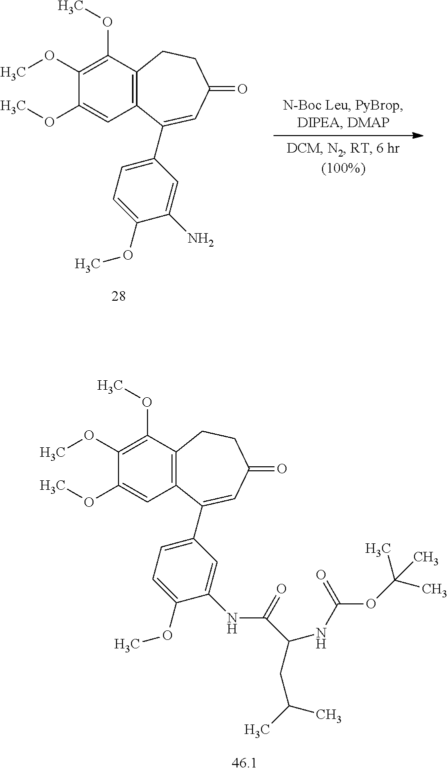 [SMILES: COc1ccc(C2=CC(=O)CCc3c2cc(OC)c(OC)c3OC)cc1N ; COc1ccc(C2=CC(=O)CCc3c2cc(OC)c(OC)c3OC)cc1NC(=O)C(CC(C)C)NC(=O)OC(C)(C)C]

To a stirred solution of 28 (400 mg, 1.09 mmol) in dry DCM (15 mL) was added a solution of N-Boc Leu (750 mg, 3.25 mmol), PyBrop (760 mg, 1.64 mmol) and DIPEA (0.56 mL, 4.36 mmol) in dry DCM (15 mL) at 0° C. The reaction temperature was allowed to increase to room temperature and left stirring for 6 hr. The reaction was then quenched by the addition of 1 M HCl (15 mL) and extracted into diethyl ether (3×15 mL). The organic fractions were combined, dried over MgSO₄, filtered and concentrated in vacuo. The product was isolated by flash column chromatography (stationary phase; silica gel 230-400 mesh, mobile phase; 3:1, hexane/ethyl acetate). All homogenous fractions were collected and the solvent was evaporated to afford 46.1 as a yellow solid (640 mg, 100%).

¹H NMR (CDCl₃) δ_{H} ppm: 0.93 (6H, t, 2×CH₃ (Leu)), 1.43 (9H, s, C(CH₃)₃), 1.55 (1H, m, CH (Leu)), 1.72 (2H, m, CH₂ (Leu)), 2.69 (2H, s, CH₂), 3.11 (2H, t, CH₂), 3.59 (3H, s, OCH₃), 3.87 (3H, s, OCH₃), 3.89 (3H, s, OCH₃), 3.91 (3H, s, OCH₃), 4.28 (1H, t, C═OCH (Leu)), 5.16 (1H, d, J=8.0 Hz, C═CH), 5.28 (1H, s, ArH (A-ring)), 6.35 (1H, d, J=2.8 Hz, ArH (C-ring)), 6.85 (1H, d, J=8.7 Hz, ArH (C-ring)), 7.03 (1H, d, J=8.2 Hz, ArH (C-ring)).

¹³C NMR (CDCl₃) δ_{c} ppm: 19.7 (CH₂), 21.4 (CH₃ (Leu)), 22.5 (CH₃ (Leu)), 24.4 (CH(CH₃)₂ (Leu)), 27.8 ((C(CH₃)₃ (Boc)), 40.7 (CH₂ (Leu)), 45.0 (CH₂), 53.5 (CHNH), 55.4 (OCH₃), 55.6 (OCH₃), 60.4 (OCH₃), 60.9 (OCH₃), 79.8 (C(CH₃)₃ (Boc)), 109.1 (ArCH), 111.6 (ArCH), 120.4 (ArCH), 124.6 (ArCH), 126.6 (ArC), 128.0 (ArCH), 128.7 (ArC), 131.9 (ArC), 135.0 (ArC), 142.8 (ArC), 148.4 (ArC), 149.4 (ArC), 150.6 (ArC), 151.5 (ArC), 155.4 (NHC═O (Boc)), 170.4 (NHC═O (Leu)), 203.5 (C═O).

νₘₐₓ/cm⁻¹: 2957.55, 1708.56, 1167.16, 842.35

### Synthesis of 46.2—Removal of tBoc Protecting Group

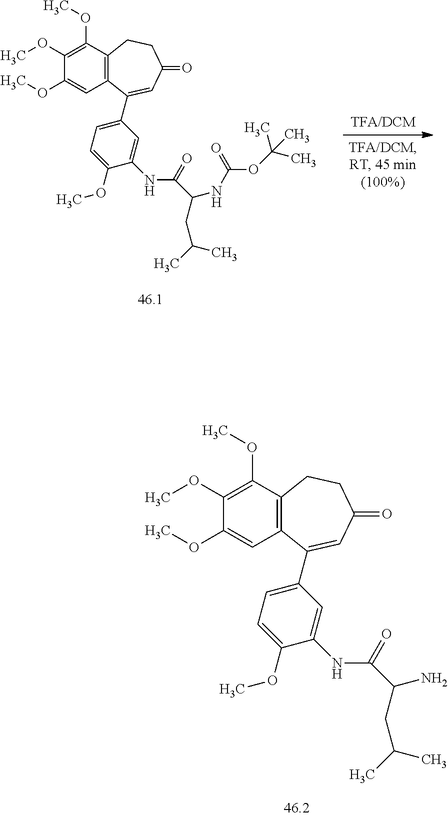

A solution of 46.1 (622 mg, 1.07 mmol) in DCM/TFA 1:1 (3 mL) was stirred for 45 min at room temperature. The reaction was quenched with aq. NaOH 2 M (5 mL) and extracted into diethyl ether (3×10 mL). The organic fractions were combined, dried over MgSO₄ and dried under vacuum. The remaining salt 46.2, a bright orange solid, was washed with diethyl ether (515 mg, 100%).

¹H NMR (CD₃OD) δ_{H} ppm: 0.99 (6H, t, 2×CH₃ (Leu)), 1.44 (H, m, CH₂ (Leu)), 1.80 (2H, t, CH (Leu), CH₂ (Leu)), 2.72 (2H, d, CH₂), 3.15 (2H, t, CH₂), 3.52 (1H, t, C═OCH (Leu)), 3.62 (3H, s, OCH₃), 3.91 (3H, s, OCH₃), 3.96 (3H, s, OCH₃), 3.97 (3H, s, OCH₃), 6.40 (2H, m, ArH (A-ring) and C═CH), 6.90 (1H, d, J=7.5, ArH (C-ring)), 7.08 (1H, d, J=7.5 Hz, ArH (C-ring)), 7.31 (1H, s, ArH (C-ring)), 8.44 (1H, br, NH).

¹³C NMR (CD₃OD) δ_{c} ppm: 20.3 (CH₂), 21.4 (CH₃ (Leu)), 23.4 (CH₃ (Leu)), 25.0 (CH(CH₃)₂ (Leu)), 44.0 (CH₂ (Leu)), 45.5 (CH₂), 54.3 (CHNH₂), 55.9 (OCH₃), 55.2 (OCH₃), 61.0 (OCH₃), 61.4 (OCH₃), 109.6 (ArCH), 112.2 (ArCH), 120.2 (ArCH), 124.6 (ArCH), 127.3 (ArC), 128.6 (ArCH), 129.2 (ArC), 132.6 (ArC), 135.6 (ArC), 143.3 (ArC), 149.1 (ArC), 150.0 (ArC), 151.0 (ArC), 152.0 (ArC), 173.7 (NHC═O (Leu)), 204.0 (C═O).

νₘₐₓ/cm⁻¹: 2936.0, 1683.2, 1524.1, 1248.2, 1113.9

HRMS: calculated 482.57, found 483.2488 (+H⁺), molecular formula (C₂₇H₃₄N₂O₆).

### Synthesis of 46.3—Coupling of N-Boc AHPA PFP Ester 44.3 to 46.2

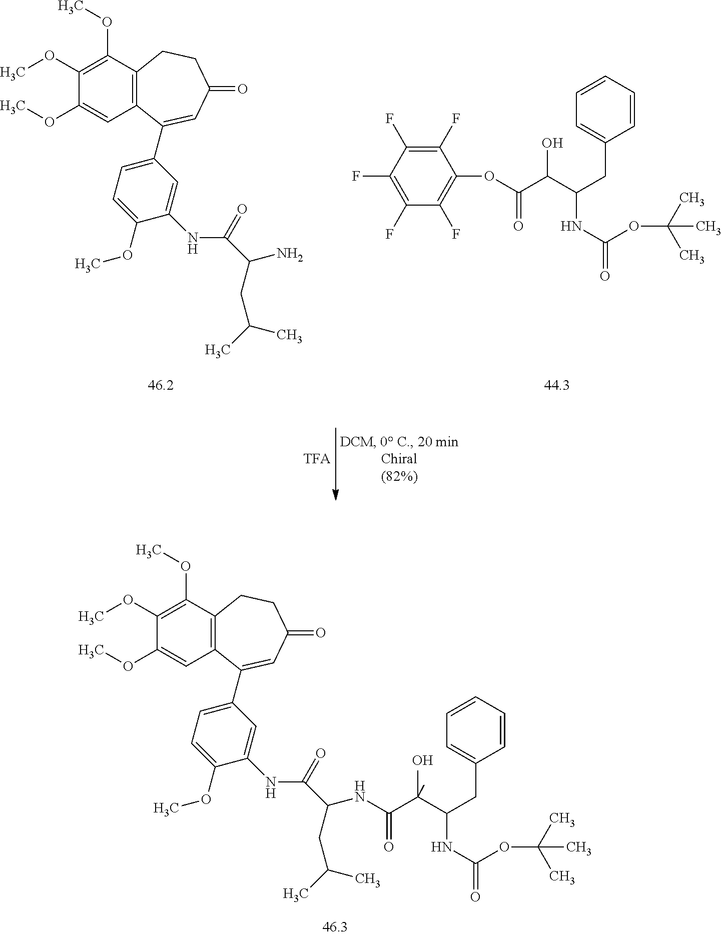

To a solution of 46.2 (70 mg, 0.15 mmol) in anhydrous DCM (1 mL) was added a solution of 44.3 (134 mg, 0.29 mmol) in anhydrous DCM (1 mL) followed by Et₃N (0.06 mL, 0.44 mmol) under an atmosphere of nitrogen at 0° C. After 20 min the solvent volume was reduced under vacuum and loaded directly onto a flash column (stationary phase; silica gel 230-400 mesh, mobile phase; 6:1, hexane/ethyl acetate). All homogenous fractions were collected and the solvent was evaporated to afford the product 46.3 as a yellow solid (93 mg, 82%).

¹H NMR (CDCl₃) δ_{H} ppm: 1.0 (6H, dd, J=6 Hz, 16.0 Hz, 2×CH₃ (Leu)), 1.40 (9H, s, C(CH₃)₃), 1.69 (1H, m, CH (Leu)), 1.73 (H, m, CH₂ (Leu)), 1.77 (H, m, CH₂ (Leu)), 2.75 (2H, m, CH₂), 3.08 (2H, m, CH₂ (AHPA bzl)), 3.16 (2H, m, CH₂), 3.64 (3H, s, OCH₃), 3.87 (3H, s, OCH₃), 3.92 (3H, s, OCH₃), 3.96 (3H, s, OCH₃), 4.21 (1H, s, CHOH (AHPA)), 4.69 (1H, dd, J=8.3 Hz, 14.0 Hz, CHNH (Leu)), 5.05 (1H, d, J=9.8 Hz, CHNH (AHPA)), 6.37 (2H, d, J=8.6 Hz, C═CH, ArH (A-ring)), 6.87 (1H, d, J=9.3 Hz, ArH (C-ring)), 7.06 (1H, dd, J=2.0 Hz, 8.0 Hz, ArH (C-ring)), 7.21-7.33 (5H, m, 5×ArH (AHPA)), 7.42 (1H, d, J=8.5 Hz, ArH (C-ring)).

¹³C NMR (CDCl₃) δ_{c} ppm: 20.3 (CH₂), 21.6 (CH₃ (Leu)), 23.1 (CH₃ (Leu)), 24.7 (CH(CH₃)₂ (Leu)), 28.2 (C(CH₃)₃ (Boc)), 35.8 (CH₂ (AHPA bzl)), 41.4 (CH₂ (Leu)), 45.7 (CH₂), 52.1 (CHNH (Leu)), 56.0 (OCH₃), 56.2 (OCH₃), 60.4 (CHNH (AHPA)), 61.0 (OCH₃), 61.2 (OCH₃), 75.6 (CHOH (AHPA)), 80.7 (C(CH₃)₃ (Boc)), 109.6 (ArCH), 112.2 (ArCH), 124.0 (ArCH), 126.7 (ArCH), 127.8 (ArCH (AHPA)), 128.4 (ArCH), 128.6 (2×ArCH (AHPA)), 129.2 (ArC), 129.3 (2×ArCH (AHPA)), 132.0 (ArC), 135.2 (ArC), 138.1 (ArC), 139.0 (ArC), 143.4 (ArC), 150.1 (ArC), 150.8 (ArC), 151.2 (ArC), 151.7 (ArC), 158.1 (NHC═O (Boc)), 170.6 (NHC═O (AHPA)), 172.8 (NHC═O (AHPA)), 204.4 (C═O).

νₘₐₓ/cm⁻¹ 2927.0, 1735.0, 1529.9, 1242.9, 1114.8

### Synthesis of 46—Removal of tBoc Group

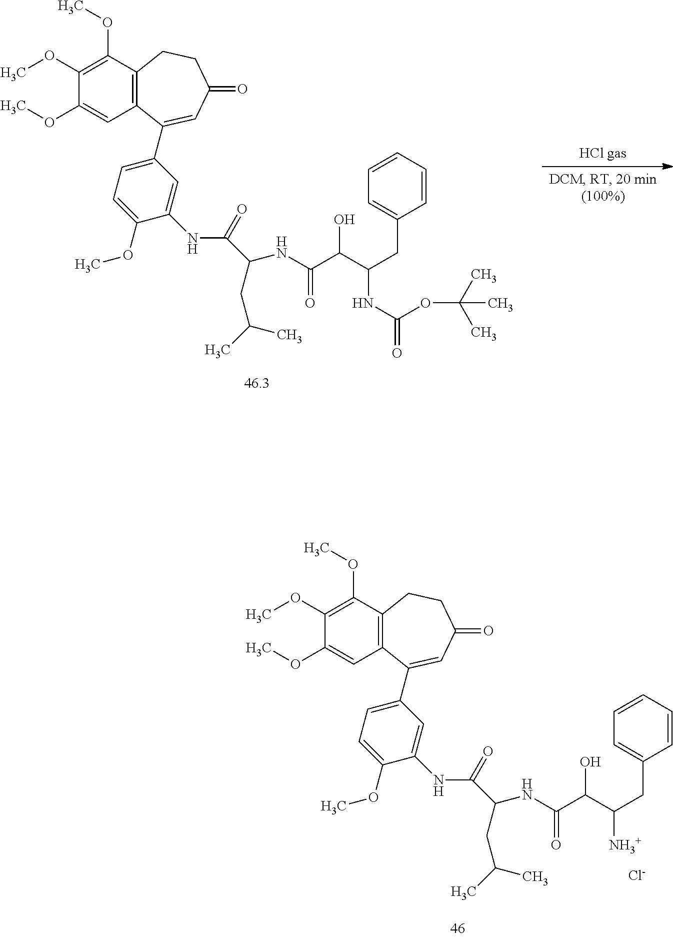

A solution of 46.3 (90 mg, 0.18 mmol) in DCM (1 mL) was acidified with HCl gas and stirred at room temperature for 20 min. Upon complete removal of the tBoc group the solvent was removed in vacuo leaving a rusty solid 46 (120 mg, 100%).

¹H NMR (CD₃OD) δ_{H} ppm: 0.92 (6H, dd, J=5 Hz, 16.6 Hz, 2×CH₃ (Leu)), 1.67 (3H, m, CH(CH₃)₂ (Leu), CH₂ (Leu)), 2.66 (2H, m, CH₂), 2.93 (2H, dd, J=8.3 Hz, 14.0 Hz, CH₂ (AHPA bzl)), 3.12 (2H, m, CH₂), 3.55 (3H, s, OCH₃), 3.84 (3H, s, OCH₃), 3.88 (3H, s, OCH₃), 3.89 (3H, s, OCH₃), 4.16 (1H, d, J=3.5 Hz, CHOH (AHPA)), 4.60 (1H, t, CHNH (Leu)), 5.48 (1H, s, CHNH (AHPA)), 6.31 (1H, d, J=4.0 Hz, C═CH), 6.41 (1H, s, ArH (A-ring)), 7.07 (1H, d, J=8.7 Hz, ArH (C-ring)), 7.13 (1H, dd, J=2.7 Hz, 8.7 Hz, ArH (C-ring)), 7.27-7.39 (6H, m, 5×ArH (AHPA), 1×ArH (C-ring)).

¹³C NMR (CD₃OD) δ_{c} ppm: 19.7 (CH₂), 20.9 (CH₃ (Leu)), 21.7 (CH₃ (Leu)), 24.6 (CH(CH₃)₂ (Leu)), 37.5 (CH₂ (AHPA bzl), 41.1 (CH₂ (Leu)), 44.9 (CH₂), 53.2 (CHNH (Leu)), 54.8 (CHNH (AHPA)), 55.0 (OCH₃), 55.3 (OCH₃), 60.0 (OCH₃), 60.6 (OCH₃), 69.6 (CHOH (AHPA)), 110.8 (ArCH), 116.1 (ArCH), 125.8 (ArCH), 129.8 (ArCH), 130.3 (ArCH), 130.4 (ArC), 131.8 (ArCH), 132.2 (2×ArCH (AHPA)), 133.0 (ArC), 133.2 (2×ArCH (AHPA)), 136.2 (ArC), 138.3 (ArC), 141.9 (ArC), 147.2 (ArC), 153.7 (ArC), 154.0 (ArC), 155.0 (ArC), 156.8 (ArC), 170.7 (NHC═O (AHPA)), 172.3 (NHC═O (AHPA)), 209.1 (C═O).

νₘₐₓ/cm⁻¹ 2932.5, 1654.4, 1492.1, 1256.3, 1114.6, 700.7

### Synthesis of compound 47

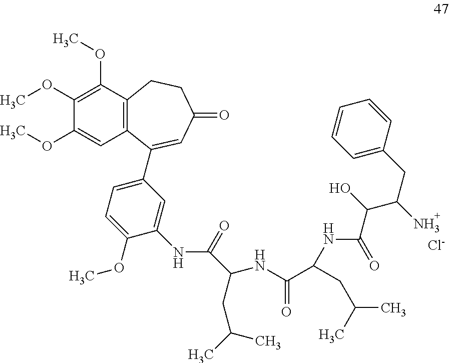 [SMILES: COc1ccc(C2=CC(=O)CCc3c2cc(OC)c(OC)c3OC)cc1NC(=O)C(CC(C)C)NC(=O)C(CC(C)C)NC(=O)C(O)C(N)Cc1ccccc1 ; [Cl-]]

### Synthesis of 3.17—N-Boc Protection of Bestatin

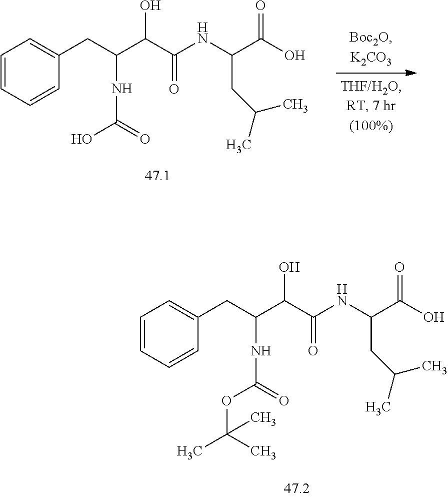 [SMILES: CC(C)CC(NC(=O)C(O)C(Cc1ccccc1)NC(=O)O)C(=O)O ; CC(C)CC(NC(=O)C(O)C(Cc1ccccc1)NC(=O)OC(C)(C)C)C(=O)O]

Bestatin 47.1 (900 mg, 2.92 mmol) and potassium carbonate (K₂CO₃) (483 mg, 3.50 mmol) was dissolved in a mixture of THF and H₂O (1:1, 40 mL). A solution of di-tert-butyldicarbonate (765 mg, 3.50 mmol) in THF/H₂O (1:1) (10 mL) was added at 0° C. The solution was stirred at room temperature for 7 hr. On completion, the solution was basified further with 2 M aq. NaOH (5 mL) and left stirring for 10 min to hydrolyse any hydroxyl-protected species. The basic solution was extracted with diethyl ether (3×10 mL) and the aqueous fraction was acidified with 2 M aq. HCl (10 mL). This was further extracted with diethyl ether (3×10 mL). The organic fractions were combined, dried over magnesium sulphate, filtered and concentrated in vacuo to afford the N-Boc protected dipeptide as a white solid 47.2 (1.2 g, 100%).

¹H NMR (CDCl₃) δ_{H} ppm: 0.91 (6H, m, 2×CH₃ (Leu)), 1.28 (9H, s, C(CH₃)₃), 1.66 (3H, m, CH₂ & CH (Leu)), 2.84 (1H, s, CH₂ (AHPA)), 3.01 (1H, d, J=6.0 Hz, CH₂ (Leu)), 4.06 (1H, s, CHNH (AHPA)), 4.18 (1H, br.d, J=17.5 Hz, CHOH), 4.60 (1H, s, CHNH (AHPA)), 5.17 (1H, d, J=77.8 Hz, NH), 6.35 (1H, br.s, OH), 7.22 (5H, m, 5×ArCH), 7.42 (1H, m, NH).

¹³C NMR (CDCl₃, 100 MHz) δ_{c} ppm: 21.4 (CH₃ (Leu)), 23.1 (CH₃ (Leu)), 24.8 (CH (Leu)), 28.2 (C(CH₃)₃), 36.6 (CH₂), 40.7 (CH₂(Leu), 50.5 (CHNH (AHPA)), 55.3 (CHNH (Leu)), 73.6 (CHOH (AHPA)), 80.4 (C(CH₃)₃), 126.5 (ArCH), 128.5 (2×ArCH), 129.3 (ArCH), 129.6 (ArCH), 138.0 (ArC), 157.2 (C═O), 173.6 (C═O), 175.7 (C═O).

νₘₐₓ/cm⁻¹: 3439.9, 2961.4, 1527.5, 1508.1, 1455.7.

### Synthesis of 47.3—Coupling of N-Boc Bestatin 47.3 to 3.1

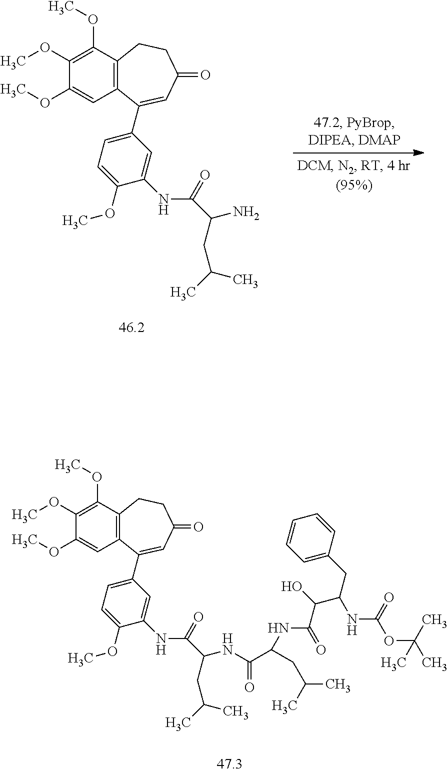 [SMILES: COc1ccc(C2=CC(=O)CCc3c2cc(OC)c(OC)c3OC)cc1NC(=O)C(N)CC(C)C ; COc1ccc(C2=CC(=O)CCc3c2cc(OC)c(OC)c3OC)cc1NC(=O)C(CC(C)C)NC(=O)C(CC(C)C)NC(=O)C(O)C(Cc1ccccc1)NC(=O)OC(C)(C)C]

To a solution of 47.2 (143 mg, 0.35 mmol), PyBrop (163 mg, 0.35 mmol) and DIPEA (0.14 mL, 0.83 mmol) in dry DCM (2 mL) was added a solution of 46.2 (100 mg, 0.21 mmol) in dry DCM (1 mL) at 0° C. under an atmosphere of nitrogen. The reaction was allowed to increase to room temperature. The pH was monitored and maintained above pH 7 with additional DIPEA. After 4 hr, the reaction mixture was applied directly to a 2phase; silica gel 230-400 mesh, mobile phase; 1:1, hexane/ethyl acetate). All homogenous fractions were collected and the solvent was evaporated to afford the product as a yellow solid 47.3 (172 mg, 95%).

¹H NMR (CDCl₃) δ_{H} ppm: 1.0 (12H, dd, J=6.6 Hz, 27.8 Hz, 4×CH₃ (Leu)), 1.40 (9H, s, C(CH₃)₃ (Boc)), 1.67 (2H, m, 2×CH (Leu)), 1.74 (2H, m, CH₂ (Leu)), 2.00 (2H, m, CH₂ (Leu)), 2.74 (2H, m, CH₂), 3.16 (2H, m, CH₂), 3.55 (2H, m, CH₂ (AHPA bzl)), 3.63 (3H, s, OCH₃), 3.92 (3H, s, OCH₃), 3.96 (3H, s, OCH₃), 3.97 (3H, s, OCH₃), 4.69 (1H, m, CHNH (AHPA)), 4.82 (2H, m, 2×CHNH (Leu)), 5.89 (1H, dd, J=6.0 Hz, 18.0 Hz, CHOH (AHPA)), 6.38 (1H, d, J=12.0 Hz, C═CH), 6.92 (1H, d, J=9.6 Hz, ArH (A-ring)), 7.10 (1H, dd, J=2.0 Hz, 8.5 Hz, ArH (C-ring)), 7.16-7.34 (5H, m, 2×ArH (C-ring), 3×ArH (AHPA)) 7.39-7.46 (2H, m, ArH (AHPA)).

¹³C NMR (CDCl₃) δ_{c} ppm: 20.3 (CH₂), 22.6 (2×CH₃ (Leu)), 23.1 (2×CH₃ (Leu)), 46.1 (CH₂ (AHPA bzl), 24.8 (CH(CH₃)₂ (Leu)), 26.4 (CH₂ (Leu)), 28.3 (C(CH₃)₃ (Boc)), 42.3 (CH₂ (Leu)), 45.7 (CH₂), 50.1 (2×CHNH (Leu)), 53.1 (CHNH (AHPA)), 56.1 (2×OCH₃), 60.9 (OCH₃), 61.4 (OCH₃), 73.0 (CHOH (AHPA)), 80.7 (C(CH₃)₃ (Boc)), 109.8 (ArCH), 112.0 (ArCH), 120.9 (ArCH), 125.9 (ArCH), 126.4 (ArC), 126.7 (ArCH), 128.6 (3×ArCH (AHPA)), 129.2 (ArC), 129.3 (2×ArCH (AHPA)), 132.3 (ArC), 135.6 (ArC), 143.4 (ArC), 148.9 (ArC), 150.1 (ArC), 151.2 (ArC), 151.6 (ArC), 156.7 (ArC), 157.0 (NHC═O (Boc)), 168.4 (NHC═O (AHPA)), 169.2 (2×NHC═O (Leu)), 204.0 (C═O). νₘₐₓ/cm⁻¹ 3276.1, 2959.8, 1647.3, 1540.6, 1274.5, 730.0

### Synthesis of 47—Removal of N-Boc Group

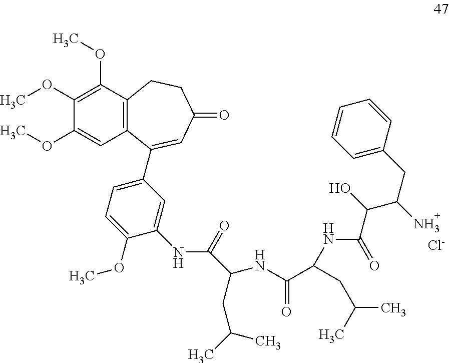 [SMILES: COc1ccc(C2=CC(=O)CCc3c2cc(OC)c(OC)c3OC)cc1NC(=O)C(CC(C)C)NC(=O)C(CC(C)C)NC(=O)C(O)C(N)Cc1ccccc1 ; [Cl-]]

A solution of 47.3 (150 mg, 0.17 mmol) in DCM (1 mL) was acidified with HCl gas and stirred at room temperature for 20 min. Upon complete removal of the tBoc group the solvent was removed in vacuo (130 mg, 100%).

¹H NMR (CD₃OD) δ_{H} ppm: 1.0 (12H, dd, J=6.6 Hz, 27.8 Hz, 4×CH₃ (Leu)), 1.67 (2H, m, 2×CH (Leu)), 1.74 (2H, m, CH₂ (Leu)), 2.00 (2H, m, CH₂ (Leu)), 2.74 (2H, m, CH₂), 3.16 (2H, m, CH₂), 3.55 (2H, m, CH₂ (AHPA bzl)), 3.63 (3H, s, OCH₃), 3.92 (3H, s, OCH₃), 3.96 (3H, s, OCH₃), 3.97 (3H, s, OCH₃), 4.69 (1H, m, CHNH (AHPA)), 4.82 (2H, m, 2×CHNH (Leu)), 5.89 (1H, dd, J=6.0 Hz, 18.0 Hz, CHOH (AHPA)), 6.38 (1H, d, J=12.0 Hz, C═CH), 6.92 (1H, d, J=9.6 Hz, ArH (A-ring)), 7.10 (1H, dd, J=2.0 Hz, 8.5 Hz, ArH (C-ring)), 7.16-7.34 (5H, m, 2×ArH (C-ring), 3×ArH (AHPA)) 7.39-7.46 (2H, m, ArH (AHPA)).

¹³C NMR (CD₃OD) δ_{c} ppm: 20.3 (CH₂), 22.6 (2×CH₃ (Leu)), 23.1 (2×CH₃ (Leu)), 46.1 (CH₂ (AHPA bzl), 24.8 (CH(CH₃)₂ (Leu)), 26.4 (CH₂ (Leu)), 42.3 (CH₂ (Leu)), 45.7 (CH₂), 50.1 (2×CHNH (Leu)), 53.1 (CHNH (AHPA)), 56.1 (2×OCH₃), 60.9 (OCH₃), 61.4 (OCH₃), 73.0 (CHOH (AHPA)), 109.8 (ArCH), 112.0 (ArCH), 120.9 (ArCH), 125.9 (ArCH), 126.4 (ArC), 126.7 (ArCH), 128.6 (3×ArCH (AHPA)), 129.2 (ArC), 129.3 (2×ArCH (AHPA)), 132.3 (ArC), 135.6 (ArC), 143.4 (ArC), 148.9 (ArC), 150.1 (ArC), 151.2 (ArC), 151.6 (ArC), 156.7 (ArC), 168.4 (NHC═O (AHPA)), 169.2 (2×NHC═O (Leu)), 204.0 (C═O).

νₘₐₓ/cm⁻¹ 3275.0, 2955.3, 1649.2, 1530.9, 1253.1, 741.0

### Synthesis of Compounds 48 and 49

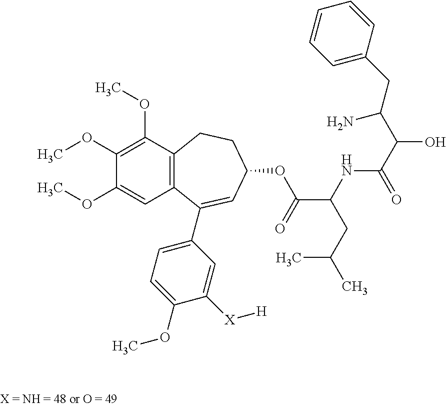

### Synthesis of 48.2—Suzuki Coupling Free Aniline

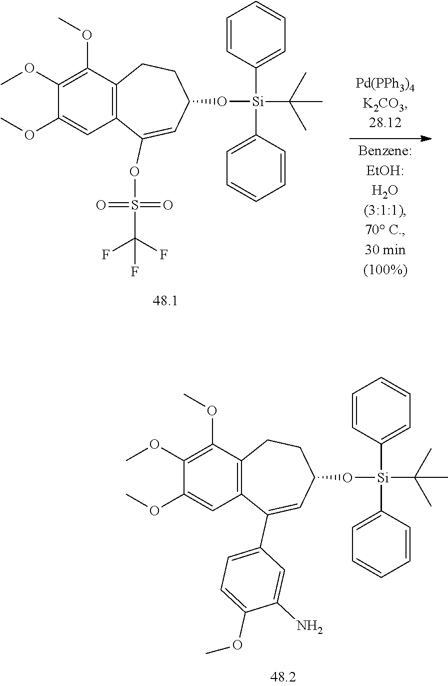

To a flask containing the stereoisomerically pure 48.1 (2.25 g, 3.54 mmol) was added 28.12 (970 mg, 3.89 mmol), K₂CO₃ (1.32 g, 9.56 mmol), and tetrakis-(triphenylphosphine)-palladium (0) (30 mg, 0.18 mmol). The mixture was dissolved in a mixture of benzene, ethanol and water (3:1:1, 10 mL). The resulting mixture was heated to 70° C. for 30 min. The reaction was quenched by the addition of water (1×20 mL) and the product was extracted with diethyl ether (3×30 mL). The organic fractions were combined, dried over MgSO₄, filtered and concentrated in vacuo. The resulting residue was purified by flash column chromatography (stationary phase; silica gel 230-400 mesh, mobile phase; 6:1, hexane/ethyl acetate). All homogenous fractions were collected and the solvent was evaporated to afford 48.2 as dark brown crystals (2.16 g, 100%).

¹H NMR (CDCl₃) δ_{H} ppm: 1.11 (9H, s, C(CH₃)₃), 2.08-2.37 (3H, m, 2×CH₂), 2.93 (1H, m, CH₂), 3.68 (3H, s, OCH₃{C-ring}), 3.74 (3H, s, OCH₃), 3.89 (3H, s, OCH₃), 3.90 (3H, s, OCH₃), 4.18 (1H, m, CHOSi), 6.22 (1H, d, J=5.0 Hz, C═CH), 6.26 (1H, s, ArH (A-ring)), 6.42 (1H, s, ArH (C-ring)), 6.50 (1H, d, J=9.0 Hz, ArH (C-ring)), 6.72 (1H, d, J=7.7 Hz, ArH (C-ring)), 7.28-7.46 (6H, m, ArH (diphenyl silyl)), 7.62-7.72 (4H, m, ArH (diphenyl silyl).

¹³C NMR (CDCl₃) δ_{c} ppm: 18.7 (C(CH₃)), 21.4 (CH₂), 26.6 (3×C(CH₃)₃), 43.4 (CH₂), 55.2 (OCH₃), 55.6 (OCH₃), 60.4 (OCH₃), 61.1 (OCH₃), 70.9 (CHOSi), 108.2 (ArCH), 109.1 (ArCH), 114.1 (ArCH), 118.1 (ArCH), 127.0 (2×ArCH), 127.1 (2×ArCH), 127.5 (ArC), 129.0 (2×ArCH), 132.0 (ArCH), 133.9 (ArC), 134.0 (ArC), 134.1 (ArC), 135.2 (ArC), 135.3 (2×ArCH), 135.5 (2×ArCH), 137.5 (ArC), 146.5 (ArC), 150.11 (ArC), 150.4 (ArC)

νₘₐₓ/cm⁻¹: 2931.09, 1488.27, 1112.69, 737.95, 702.93

HRMS: calculated 609.83, found 610.3036 (+H⁺), molecular formula (C₃₇H₄₃NO₅Si).

### Synthesis of 48.3—Removal of Silyl Group

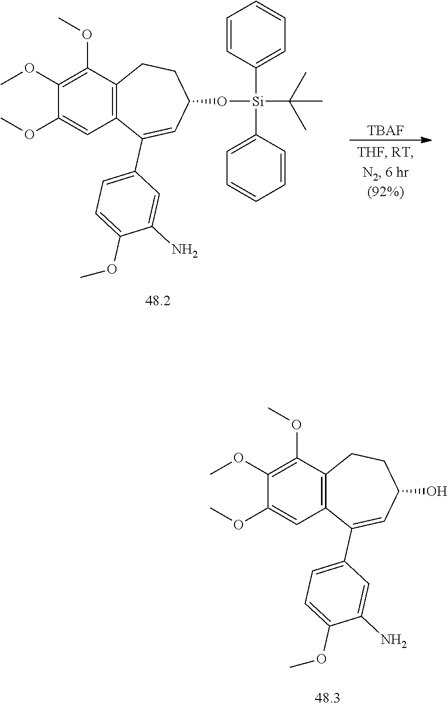

To a stirred solution of 48.2 (2.6 g, 4.2 mmol) in THF (15 mL) was added 1 M TBAF (4.2 mL, 4.2 mmol) at 0° C. The reaction was brought to room temperature. After 6 hr the reaction mixture was applied directly to a flash column. The product was purified by flash column chromatography (stationary phase; silica gel 230-400 mesh, mobile phase; 2:1, hexane/ethyl acetate). All homogenous fractions were collected and the solvent was evaporated to afford 48.3 as a golden solid (1.43 g, 92%).

¹H NMR (CDCl₃) δ_{H} ppm: 2.12 (1H, m, CH₂), 2.35 (1H, m, CH₂), 2.52 (1H, m, CH₂), 3.03 (1H, m, CH₂), 3.70 (3H, s, OCH₃), 3.88 (3H, s, OCH₃), 3.91 (3H, s, OCH₃), 3.93 (3H, s, OCH₃), 4.14 (1H, m, CHOH), 6.25 (1H, d, J=5.0 Hz, C═CH), 6.40 (1H, s, ArH (A-ring)), 6.70 (1H, d, J=2.0 Hz, 8.5 Hz, ArH (C-ring)), 6.75 (1H, d, J=2.0 Hz, ArH (C-ring)), 6.88 (1H, m, ArH (C-ring)).

¹³C NMR (CDCl₃) δₑ ppm: 21.6 (CH₂), 43.2 (CH₂), 55.1 (OCH₃), 55.6 (OCH₃), 60.4 (OCH₃), 61.1 (OCH₃), 69.7 (CHOH), 108.3 (ArCH), 109.6 (ArCH), 114.3 (ArCH), 117.9 (ArCH), 127.5 (ArC), 130.7 (ArCH), 133.7 (ArC), 135.0 (ArC), 135.3 (ArC), 138.5 (ArC), 140.9 (ArC), 146.6 (ArC), 150.2 (ArC), 150.6 (ArC).

νₘₐₓ/cm⁻¹: 2960.67, 2933.63, 2855.96, 1593.66, 1487.76, 1235.81, 1112.45, 704.20

HRMS: calculated 371.43, found 394.1603 (+Na⁺), molecular formula (C₂₁H₂₅NO₅).

### Synthesis of 48.4—Selective Protection of the Aniline

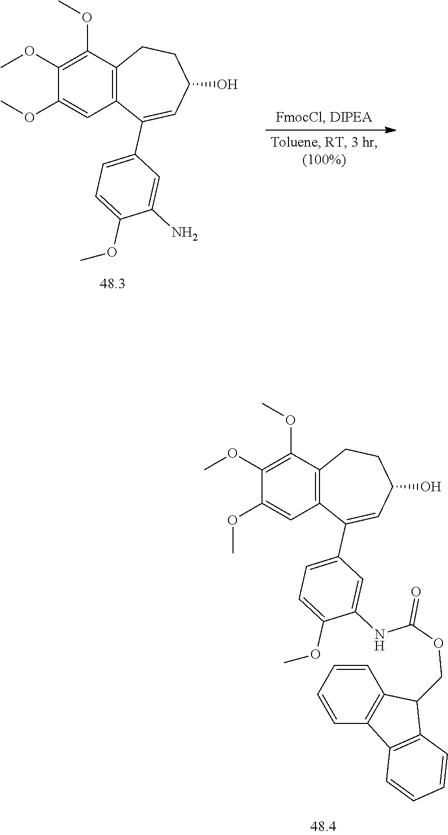 [SMILES: COc1ccc(C2=C[C@@H](O)CCc3c2cc(OC)c(OC)c3OC)cc1N ; COc1ccc(C2=C[C@@H](O)CCc3c2cc(OC)c(OC)c3OC)cc1NC(=O)OCC1c2ccccc2-c2ccccc21]

To a solution of 48.3 (5.06 g, 13.6 mmol) and DIPEA (4.76 mL, 28.5 mmol) in toluene (25 mL) was added Fmoc chloride (7.05 g, 27.2 mmol) in toluene (20 mL). After 3 hr at room temperature the solvent was removed under vacuum and the product was isolated by flash column chromatography (stationary phase; silica gel 230-400 mesh, mobile phase; 2:1, hexane/ethyl acetate). All homogenous fractions were collected and the solvent was evaporated to afford 48.4 as an off-white solid (8.09 g, 100%).

¹H NMR (CDCl₃) δ_{H} ppm: 2.14 (1H, m, CH₂), 2.39 (1H, m, CH₂), 2.54 (1H, m, CH₂), 3.05 (1H, dd, J=5.8 Hz, 12.8 Hz, CH₂), 3.70 (3H, s, OCH₃), 3.92 (3H, s, OCH₃), 3.94 (6H, s, 2×OCH₃), 4.19 (1H, m, CHOH), 4.33 (1H, t, CH (Fmoc)), 4.51 (2H, q, CH₂ (Fmoc)), 6.36 (1H, d, J=5.0 Hz, C═CH), 6.40 (1H, s, ArH (A-ring)), 6.83 (1H, d, J=2.0 Hz, 8.5 Hz, ArH (C-ring)), 6.90 (1H, d, J=2.0 Hz, ArH (C-ring)), 7.29 (1H, d, J=8.0 Hz, ArH (C-ring)), 7.36 (2H, t, ArH (Fmoc)), 7.45 (2H, t, ArH (Fmoc)), 7.66 (2H, d, J=7.4 Hz, ArH (Fmoc)), 7.81 (2H, d, J=7.7 ArH (Fmoc)), 8.22 (2H, br, NH₂), ¹³C NMR (CDCl₃) δ_{c} ppm: 21.9 (CH₂), 43.5 (CH₂), 47.2 (CH (Fmoc)), 55.9 (OCH₃), 56.1 (OCH₃), 60.9 (OCH₃), 61.6 (OCH₃), 67.1 (CH₂ (Fmoc)), 69.9 (CHOH), 108.9 (ArCH), 109.7 (ArCH), 120.1 (2×ArCH), 123.1 (ArCH), 125.1 (2×ArCH (Fmoc)), 127.1 (2×ArCH (Fmoc)), 127.3 (ArC), 127.8 (2×ArCH (Fmoc)), 128.2 (2×ArC (Fmoc)), 132.2 (2×ArC (Fmoc)), 134.3 (ArC), 135.2 (ArC), 138.8 (ArC), 141.3 (2×ArC (Fmoc)), 141.5 (ArC), 143.8 (ArC), 143.8 (ArC), 150.8 (ArC), 151.1 (ArC), 153.4 (C═O).

νₘₐₓ/cm⁻¹: 1264.48, 732.70, 702.92

HRMS: calculated 593.66, found 616.2303 (+Na⁺), molecular formula (C₃₆H₃₅NO₇).

### Synthesis of 48.5—Coupling Fmoc Leu to 48.4 (Aniline) and Synthesis of 49.2—Coupling of Fmoc Leu to 49.1 (Phenol)

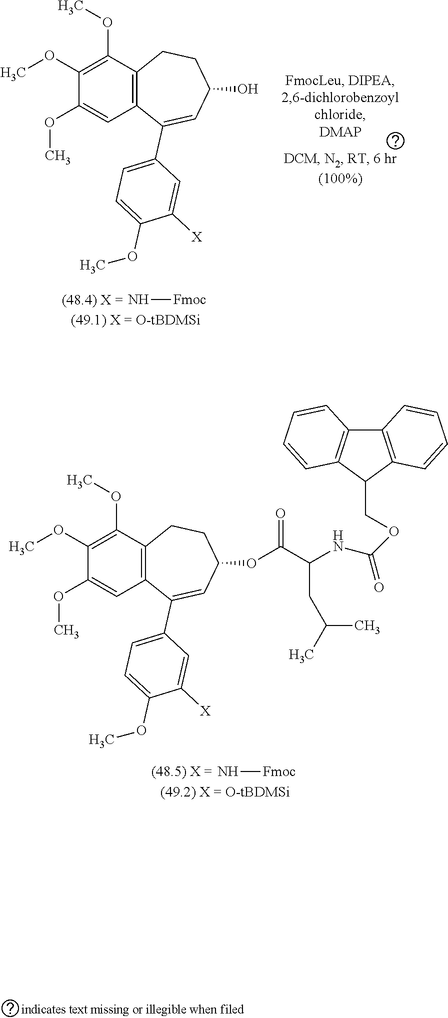

### Synthesis of 48.5—Coupling Fmoc Leu to 48.4 (Aniline)

To a solution of Fmoc-Leu (300 mg, 0.84 mmol), DIPEA (0.20 mL, 1.19 mmol) and 2,6-dichlorobenzoyl chloride (0.12 mL, 0.84 mmol) in dry DCM (5 mL) was added a solution of 48.4 (100 mg, 0.17 mmol) and DMAP (20 mg, 0.17 mmol) in dry DCM (10 mL) under an atmosphere of nitrogen at 0° C. The reaction was brought to room temperature and left stirring for 6 hr. After this time the reaction mixture was applied directly to a flash column and the product was isolated by flash column chromatography (stationary phase; silica gel 230-400 mesh, mobile phase; 6:1, hexane/ethyl acetate). All homogenous fractions were collected and the solvent was evaporated to afford the product 48.5 as a yellow oil (160 mg, 100%).

¹H NMR (CDCl₃) δ_{H} ppm: 0.99 (6H, d, J=6.2 Hz, 2×CH₃ (Leu)), 1.58 (1H, m, CH₂ (Leu)), 1.67 (H, m, CH₂ (Leu)), 1.71 (H, m, CH (Leu)), 2.23 (1H, m, CH₂), 2.49 (1H, m, CH₂), 2.64 (1H, m, CH₂), 3.08 (1H, dd, J=5.0 Hz, 16.5 Hz, CH₂), 3.72 (3H, s, OCH₃), 3.94 (6H, s, 2×OCH₃), 3.97 (3H, s, OCH₃), 4.29 (2H, m, 2×CH(Fmoc)), 4.42 (1H, t, CHNH (Leu)), 4.45 (4H, m, 2×CH₂ (Fmoc)), 5.26 (1H, m, CHC═C), 6.24 (1H, d, J=4.7 Hz, C═CH (B-ring)), 6.43 (1H, s, ArH (A-ring)), 6.84 (1H, d, J=8.4 Hz, ArH (C-ring)), 6.93 (1H, d, J=7.7 Hz, ArH (C-ring)), 7.45-7.25 (1H, m, ArH (C-ring)), 8H, ArH (Fmoc)), 7.64 (4H, t, ArH (Fmoc)), 7.80 (4H, dd, J=7.7 Hz, 13.6 Hz, ArH(Fmoc)), 8.17 (2H, br, 2×NH).

¹³C NMR (CDCl₃) δ_{c} ppm: 21.6 (CH₂), 22.0 (CH₃ (Leu)), 22.8 (CH₃ (Leu)), 24.9 (CH (Leu)), 40.2 (CH₂), 41.7 (CH₂(Leu)), 47.1 (CH (Fmoc)), 47.2 (CH (Fmoc)), 52.7 (CHN (Leu)), 55.8 (OCH₃), 56.1 (OCH₃), 60.9 (OCH₃), 61.6 (OCH₃), 66.4 (2×CH₂ (Fmoc)), 73.3 (CHO), 109.1 (ArCH), 109.7 (ArCH), 120.0 (2×ArCH (Fmoc)), 120.1 (2×ArCH (Fmoc)), 123.2 (ArCH), 125.1 (2×ArCH (Fmoc)), 125.2 (2×ArCH (Fmoc)), 126.6 (ArCH), 127.1 (2×ArCH (Fmoc)), 127.2 (2×ArCH (Fmoc)), 127.3 (ArC), 127.6 (ArC), 127.7 (2×ArCH (Fmoc)), 127.8 (2×ArCH (Fmoc)), 134.0 (ArC), 134.8 (ArC), 140.5 (2×ArC (Fmoc)), 141.3 (2×ArC (Fmoc)), 141.8 (2×ArC (Fmoc)), 143.8 (2×ArC (Fmoc)), 144.0 (ArC), 147.7 (ArC), 150.9 (ArC), 151.4 (ArC), 153.3 (ArC), 156.0 (2×NC═O (Fmoc)), 172.4 (OC═O).

νₘₐₓ/cm⁻¹: 2957.94, 1715.85, 1430.21, 739.10.

### Synthesis of 49.2—Coupling of Fmoc Leu to 49.1 (Phenol)

To a solution of Fmoc-Leu (12.0 g, 34.0 mmol), DIPEA (8.1 mL, 47.6 mmol) and 2,6-dichlorobenzoyl chloride (4.9 mL, 34.0 mmol) in dry DCM (10 mL) was added a solution of 49.1 (3.31 g, 6.8 mmol) and DMAP (830 mg, 6.8 mmol) in dry DCM (10 mL) under an atmosphere of nitrogen at 0° C. The reaction was brought to room temperature and left stirring for 6 hr. After this time the reaction mixture was applied directly to a flash column and the product was isolated by flash column chromatography (stationary phase; silica gel 230-400 mesh, mobile phase; 6:1, hexane/ethyl acetate). All homogenous fractions were collected and the solvent was evaporated to afford the product 49.2 as a golden oil (5.6 g, 100%).

¹H NMR (CDCl₃) δ_{H} ppm: 0.21 (3H, s, SiCH₃), 0.22 (3H, s, SiCH₃), 1.04 (6H, s, 2×CH₃ (Leu)), 1.05 (9H, s, C(CH₃)₃ (tBDMS)), 1.63 (2H, m, CH₂ (Leu)) 1.74 (1H, m, CH(CH₃)₂ (Leu)), 2.26 (1H, m, CH₂), 2.48 (1H, m, CH₂), 2.65 (1H, m, CH₂), 3.12 (1H, m, CH₂), 3.72 (3H, s, OCH₃ {C-ring}), 3.86 (3H, s, OCH₃), 3.97 (3H, s, OCH₃), 3.98 (3H, s, OCH₃), 4.25 (1H, t, CH(Fmoc)), 4.43 (1H, t, CHNH (Leu)), 4.46 (2H, m, CH₂ (Fmoc)), 5.27 (1H, m, CHO), 6.16 (1H, d, J=4.9 Hz, C═CH), 6.43 (1H, s, ArH {A-ring}), 6.85 (3H, m, ArH {C-ring}), 7.33 (2H, m, 2×ArH (Fmoc)), 7.42 (2H, t, 2×ArH (Fmoc)), 7.65 (2H, m, 2×ArH (Fmoc)), 7.79 (2H, d, J=7.5, 2×ArH (Fmoc)).

¹³C NMR (CDCl₃) δ_{c} ppm: −4.8 (Si(CH₃)₂), 18.5 (C(CH₃)₃), 21.6 (CH₂), 22.0 (CH₃ (Leu)), 22.9 (CH₃ (Leu)), 24.8 (CH(CH₃)₂ (Leu)), 25.8 (3×C(CH₃)₃ (tBDMS)), 40.3 (CH₂), 41.7 (CH₂ (Leu)), 47.4 (CH (Fmoc)), 51.8 (CHNH), 52.6 (CH (Leu)), 55.5 (OCH₃), 55.9 (OCH₃), 61.0 (OCH₃), 61.6 (OCH₃), 66.8 (CH₂ (Fmoc)), 73.5 (CHO), 109.0 (ArCH), 111.7 (ArCH), 120.1 (2×ArCH), 120.8 (ArCH), 121.7 (ArCH), 125.2 (ArCH), 126.0 (ArCH), 127.1 (2×ArCH), 127.8 (2×ArCH), 129.5 (ArCH), 130.5 (ArC), 133.9 (ArC), 135.0 (ArC), 138.8 (ArC), 140.5 (ArC), 141.3 (ArC), 141.8 (ArC), 143.8 (ArC), 144.0 (ArC), 144.7 (ArC), 150.9 (ArC), 151.4 (ArC), 156.1 (ArC), 171.2 (C═O), 173.5 (C═O).

νₘₐₓ/cm⁻¹ 3411.4, 2931.7, 1596.0

### Synthesis of 48.6—Removal of Fmoc from 48.5 and Synthesis of 49.3—Removal of Fmoc from 49.2

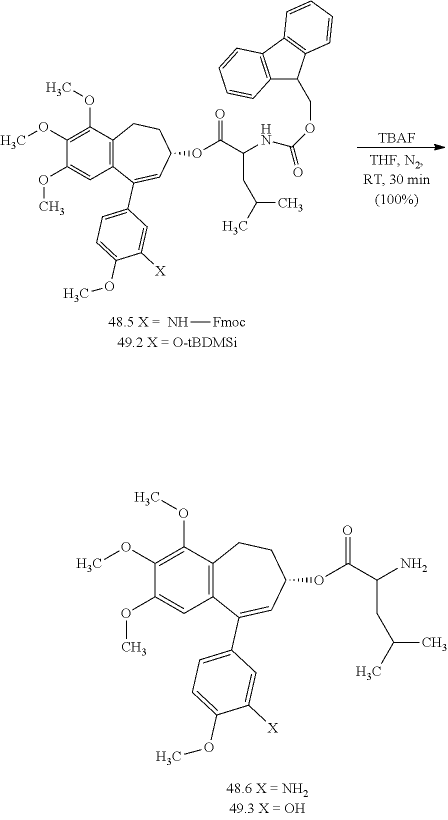

### Synthesis of 48.6—Removal of Fmoc from 48.5

To a solution of 48.5 (160 mg, 0.17 mmol) in anhydrous THF (0.9 mL), was added 1 M TBAF (0.40 mL, 0.40 mmol) at 0° C. The reaction was shown by TLC to be complete after 30 min. The reaction mixture was applied directly to a flash column and the product was isolated by flash column chromatography (stationary phase; silica gel 230-400 mesh, mobile phase; 10:1:1, ethyl acetate/methanol/diethylamine). All homogenous fractions were collected and the solvent was evaporated to afford the product as a white solid 48.6 (82 mg, 100%).

¹H NMR (CD₃OD) δ_{H} ppm: 0.96 (6H, q, 2×CH₃ (Leu)), 1.41 (H, m, CH₂), 1.50 (H, m, CH₂), 1.78 (H, m, CH (Leu)), 2.17 (1H, m, CH₂ (Leu)), 2.44 (1H, m, CH₂), 2.60 (1H, m, CH₂ (Leu)), 3.05 (1H, qd, J=2.3 Hz, CH₂), 3.48 (1H, dd, J=5.5 Hz, 9.1 Hz, CHNH (Leu)), 3.71 (3H, s, OCH₃), 3.89 (3H, s, OCH₃), 3.93 (3H, s, OCH₃), 3.94 (3H, s, OCH₃), 5.14 (1H, m, CHC═C), 6.10 (1H, d, J=5.3 Hz, C═CH), 6.39 (1H, s, ArH (A-ring)), 6.64 (H, d, J=6.8 Hz, ArH (C-ring), 6.65 (1H, s, ArH (C-ring)), 6.72 (H, d, J=8.4 Hz, ArH (C-ring)).

¹³C NMR (CD₃OD) δ_{c} ppm: 21.6 (CH₂), 21.9 (CH₃ (Leu)), 22.9 (CH₃ (Leu)), 24.9 (CH (Leu)), 40.5 (CH₂(Leu)), 43.7 (CH₂), 52.7 (CHN (Leu)), 55.8 (OCH₃), 56.1 (OCH₃), 60.9 (OCH₃), 61.6 (OCH₃), 73.3 (CHO), 109.1 (2×ArCH), 109.7 (ArCH), 123.2 (ArCH), 126.6 (ArCH), 127.3 (ArC), 127.6 (ArC), 134.0 (ArC), 134.8 (ArC), 144.0 (ArC), 147.7 (ArC), 150.9 (ArC), 151.4 (ArC), 153.3 (ArC), 172.4 (OC═O).

νₘₐₓ/cm⁻¹ 2931.5, 1508.1, 1246.6, 1113.0, 700.9

### Synthesis of 49.3—Removal of Fmoc from 49.2

To a stirred solution of 49.2 (3.8 g, 4.6 mmol) in anhydrous THF was added 1 M TBAF (9.24 mL, 9.24 mmol) at 0° C. under an atmosphere of nitrogen. The reaction was complete within 30 min and the mixture was applied directly to flash column (stationary phase; silica gel 230-400 mesh, mobile phase; 6:1:1, ethyl acetate/methanol/diethylamine). All homogenous fractions were collected and the solvent was evaporated. The product 49.3 was isolated as an off-white solid (2.2 g, 100%).

¹H NMR (CD₃OD) δ_{H} ppm: 0.90 (6H, q, 2×CH₃ (Leu)), 1.40 (H, m, CH₂ (Leu)), 1.56 (H, m, CH₂ (Leu)), 1.74 (H, m, CH (Leu)), 2.12 (1H, m, CH₂), 2.38 (1H, m, CH₂), 2.52 (1H, m, CH₂), 3.00 (1H, qd, J=2.0 Hz, CH₂), 3.42 (1H, m, CHNH (Leu)), 3.63 (3H, s, OHH₃), 3.81 (3H, s, OHH₃), 3.86 (3H, s, OCH₃), 3.88 (3H, s, OCH₃), 5.10 (1H, m, CHO), 6.10 (1H, d, J=5.3 Hz, C═CH), 6.34 (1H, s, ArH (A-ring)), 6.72 (H, d, J=2.0 Hz, ArH (C-ring), 6.74 (1H, s, ArH (C-ring)), 6.84 (H, d, J=1.9 Hz, ArH (C-ring)).

¹³C NMR (CD₃OD) δ_{c} ppm: 21.5 (CH₂), 21.9 (CH₃ (Leu)), 22.9 (CH₃ (Leu)), 24.9 (CH (Leu)), 40.2 (CH₂), 43.5 (CH₂ (Leu)), 52.7 (CHN (Leu)), 55.8 (OCH₃), 56.1 (OCH₃), 60.9 (OCH₃), 61.6 (OCH₃), 72.5 (CHO), 109.1 (ArCH), 110.5 (ArCH), 114.9 (ArCH), 119.8 (ArCH), 126.5 (ArCH), 127.4 (ArC), 134.3 (ArC), 135.1 (ArC), 140.2 (ArC), 141.8 (ArC), 145.6 (ArC), 147.0 (ArC), 150.8 (ArC), 151.2 (ArC), 175.6 (OC═O).

νₘₐₓ/cm⁻¹ 3401.5, 2974.3, 1697.0, 1507.9, 1166.5, 1098.3, 699.0

### Synthesis of 4.11—Fmoc Protection of AHPA (2.23)

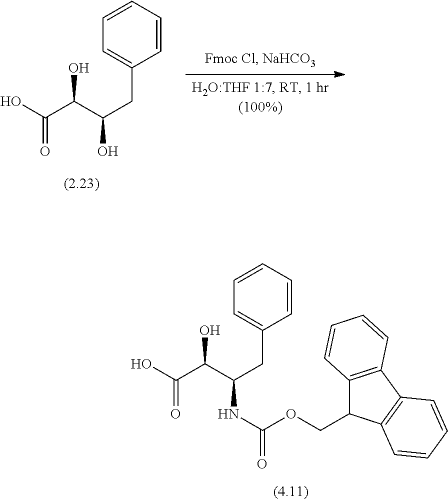 [SMILES: O=C(O)[C@@H](O)[C@H](O)Cc1ccccc1 ; O=C(N[C@H](Cc1ccccc1)[C@H](O)C(=O)O)OCC1c2ccccc2-c2ccccc21]

### Synthesis of 4.11—Fmoc Protection of AHPA (2.23)

To a solution of AHPA (100 mg, 0.51 mmol) and potassium carbonate (78 mg, 0.51 mmol) in water (3 mL) and THF (2 mL) was added a solution of Fmoc chloride (132 mg, 0.51 mmol) in THF (4 mL) at 0° C. The temperature was brought to room temperature. After 1 hr, the organic solvent was removed in vacuo and the alkaline aqueous product was washed with diethyl ether (3×30 mL). The aqueous fraction was then acidified with 2 M HCl (30 mL) and the product was extracted into diethyl ether (3×30 mL). The organic layers were combined, dried over magnesium sulphate, filtered and concentrated to afford 4.11 as a white solid (210 mg, 100%).

¹H NMR (CD₃OD) δ_{H} ppm: 2.92 (2H, dm, CH₂), 4.09 (1H, m, CHOH), 4.11 (1H, m, CH (Fmoc)), 4.15 (1H, m, CH₂ (Fmoc)), 4.24 (1H, m, CH₂ (Fmoc)), 4.28 (1H, m, CHNH), 7.17 (H, d, J=7.2 Hz, ArH (AHPA)), 7.28 (6H, m, 4×ArH (AHPA), 2×ArH (Fmoc)), 7.37 (2H, t, 2×ArH (Fmoc)), 7.56 (2H, m, 2×ArH (Fmoc)), 7.75 (2H, d, J=7.6 Hz, 2×ArH (Fmoc)).

¹³C NMR (CD₃OD) δ_{c} ppm: 37.8 (CH₂), 46.6 (CH (Fmoc)), 55.5 (CHNH (AHPA)), 66.6 (CH₂ (Fmoc)), 70.5 (CHOH (AHPA)), 119.7 (2×ArCH), 125.0 (2×ArCH), 126.3 (ArCH), 126.9 (2×ArCH), 127.5 (2×ArCH), 128.2 (2×ArCH), 129.3 (2×ArCH), 138.0 (ArC), 141.2 (2×ArC), 143.9 (2×ArC), 156.7 (NHC═O (Fmoc)), 175.5 (C=0 (AHPA)).

νₘₐₓ/cm⁻¹ 3304.2, 2953.1, 1692.1, 1509.0, 1246.9, 1112.1, 740.0

### Synthesis of 4.12—PFP Ester of 4.11

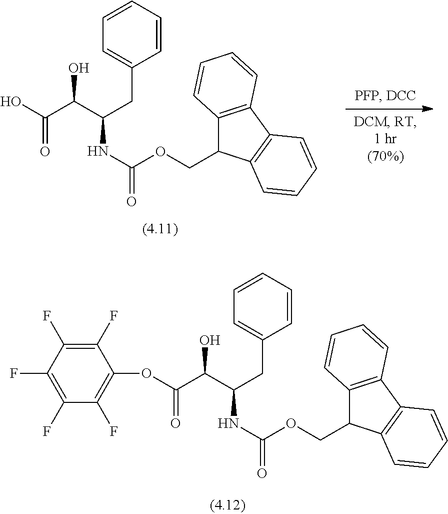 [SMILES: O=C(N[C@H](Cc1ccccc1)[C@H](O)C(=O)O)OCC1c2ccccc2-c2ccccc21 ; O=C(N[C@H](Cc1ccccc1)[C@H](O)C(=O)Oc1c(F)c(F)c(F)c(F)c1F)OCC1c2ccccc2-c2ccccc21]

To a stirred solution of Fmoc AHPA (1.46 g, 3.5 mmol) in anhydrous DCM/DMF (7 mL/3 mL) was added pentafluorophenol (770 mg, 4.2 mmol) in dry DCM (5 mL) followed by DCC (1.44 g, 7.0 mmol) in dry DCM (5 mL) at 0° C. under an atmosphere of nitrogen. The temperature was allowed to increase to ambient over one hour. The reaction was then filtered to remove the byproduct DCU and the product was purified by flash column chromatography (stationary phase; silica gel 230-400 mesh, mobile phase; 6:1, hexane/ethyl acetate). All homogenous fractions were collected and the solvent was evaporated to afford an opaque sticky semi-solid (1.43 g, 70%).

¹H NMR (CDCl₃) δ_{H} ppm: 2.92 (2H, dm, CH₂), 4.07 (H, t, CHOH), 4.20 (2H, m, CHNH, CH (Fmoc)), 4.41 (2H, t, CH₂ (Fmoc)), 7.14 (5H, m, 5×ArH (AHPA)), 7.24 (4H, m, 4×ArH (Fmoc)), 7.43 (2H, t, 2×ArH (Fmoc)), 7.62 (2H, m, 2×ArH (Fmoc)).

¹³C NMR (CDCl₃) δ_{c} ppm: 37.4 (CH₂), 46.6 (CH (Fmoc)), 55.0 (CHNH), 66.6 (CH₂ (Fmoc)), 70.2 (CHOH (AHPA)), 119.3 (2×ArCH), 124.5 (ArCH), 124.6 (ArCH), 126.1 (ArCH), 126.4 (2×ArCH), 127.0 (2×ArCH), 128.0 (2×ArCH), 128.9 (2×ArCH), 136.9 (3×ArC), 140.7 (2×ArC), 143.2 (ArC), 143.4 (3×ArC), 155.6 (2×ArC), 158.9 (NHC═O (Fmoc)),), 169.0 (C=0 (AHPA)).

νₘₐₓ/cm⁻¹ 3321.1, 2929.9, 1626.1, 1515.4, 1243.8, 980.8, 739.4

### Synthesis of 48.7—Coupling of 4.12 to 48.6 and Synthesis of 49.4 by Coupling of 4.12 to 49.3

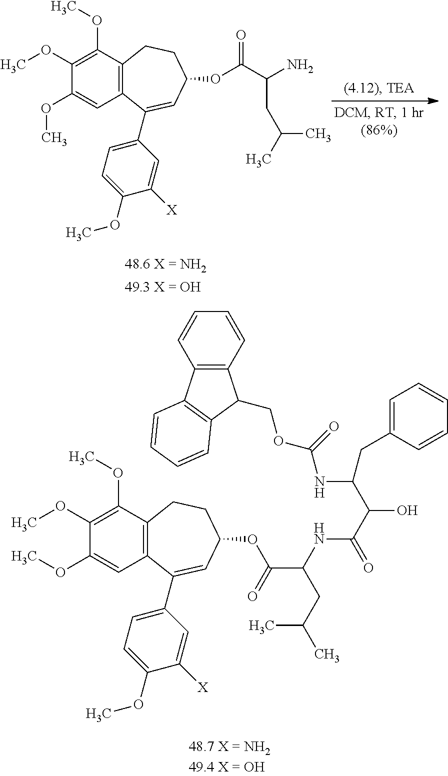

Synthesis of 48.7—Coupling of 4.12 to 48.6 and Synthesis of 49.4—Coupling of 4.12 to 49.3.

To a stirred solution of 48.6 (200 mg, 0.41 mmol) in anhydrous DCM (2 mL) was added 4.12 (480 mg, 0.82 mmol) followed by triethylamine (0.11 mL, 0.82 mmol) at 0° C. under an atmosphere of nitrogen. The reaction was allowed to reach ambient temperature over one hour after which time it was reduced in volume under vacuum and loaded directly onto a flash column (stationary phase; silica gel 230-400 mesh, mobile phase; 2:1, hexane/ethyl acetate). All homogenous fractions were collected and the solvent was evaporated. The product 48.7 was isolated as a red solid (310 mg, 86%).

¹H NMR (CDCl₃) δ_{H} ppm: 0.90 (6H, d, J=6.2 Hz, 2×CH₃ (Leu)), 1.60 (3H, m, CH₂ (Leu), CH (Leu)), 1.92 (1H, m, CH₂), 2.21 (1H, m, CH₂), 2.45 (1H, m, CH₂), 2.58 (1H, m, CH₂ (AHPA)), 2.94 (1H, m, CH₂ (AHPA)), 3.04 (1H, m, CH₂), 3.65 (3H, s, OCH₃), 3.86 (3H, s, OCH₃), 3.90 (3H, s, OCH₃), 3.94 (3H, s, OCH₃), 4.25 (4H, m, CHOH (AHPA), CH (Fmoc)), CH₂ (Fmoc)), 4.63 (H, m, CHNH (Leu)), 5.17 (H, m, CHNH (AHPA), 5.74 (H, d, J=6.5 Hz, CHC═C), 6.12 (1H, d, J=5.0 Hz, C═CH (B-ring)), 6.38 (1H, s, ArH (A-ring)), 6.67 (1H, s, ArH (C-ring)), 6.65-6.73 (2H, dd, J=9.0 Hz, 29.0 Hz, 2×ArH (C-ring)), 7.18 (2H, t, 2×ArH (AHPA)), 7.24 (2H, t, 2×ArH (Fmoc)), 7.29 (3H, t, 3×ArH (AHPA)), 7.40 (2H, t, 2×ArH (Fmoc)), 7.51 (2H, t, 2×ArH (Fmoc)), 7.76 (2H, d, J=8.0 Hz, 2×ArH (Fmoc)).

¹³C NMR (CDCl₃) δ_{c} ppm: 20.9 (CH₃ (Leu)), 21.4 (CH₂), 21.8 (CH₃ (Leu)), 23.0 (CH (Leu)), 36.5 (CH₂ (AHPA)), 40.2 (CH₂ (Leu)), 41.4 (CH₂), 47.2 (CH (Fmoc)), 46.8 (CHNH (AHPA)), 55.5 (OCH₃), 56.0 (OCH₃), 56.6 (CHN (Leu)), 60.8 (OCH₃), 61.6 (OCH₃), 67.2 (CH₂ (Fmoc)), 73.0 (CHOH (AHPA)), 73.4 (CHC═C), 109.1 (ArCH (A-ring)), 110.0 (ArCH), 114.7 (ArCH), 118.5 (ArCH), 120.0 (2×ArCH (Fmoc)), 125.2 (2×ArCH (Fmoc)), 125.7 (ArCH (AHPA)), 126.6 (ArCH), 127.1 (2×ArCH (Fmoc)), 127.4 (ArC), 127.7 (2×ArCH (Fmoc)), 128.6 (2×ArCH (AHPA)), 129.3 (2×ArCH (AHPA)), 134.0 (ArC), 135.2 (ArC), 135.9 (ArC), 137.9 (ArC), 140.9 (ArC), 141.3 (2×ArC (Fmoc)), 141.6 (ArC), 143.7 (ArC (Fmoc)), 143.8 (ArC (Fmoc)), 147.4 (ArC), 150.9 (ArC), 151.3 (ArC), 157.1 (NHC═O (Fmoc)), 171.4 (OC═P (AHPA)), 172.4 (OC═O (Leu)).

νₘₐₓ/cm⁻¹2932.6, 1655.0, 1511.5, 1236.8, 1112.7, 1080.7, 739.7

### Synthesis of 49.4—Coupling of 4.12 to 49.3

To a stirred solution of 49.3 (1.75 g, 3.6 mmol) in anhydrous DCM (10 mL) was added (4.12) (2.4 g, 4.1 mmol) followed by triethylamine (0.6 mL, 4.1 mmol) at 0° C. under an atmosphere of nitrogen. The reaction was allowed to reach ambient temperature over one hour after which time it was reduced in volume under vacuum and loaded directly onto a flash column (stationary phase; silica gel 230-400 mesh, mobile phase; 2:1, hexane/ethyl acetate). All homogenous fractions were collected and the solvent was evaporated. The product 49.4 was isolated as an off-white solid (2.75 g, 86%).

¹H NMR (CDCl₃) δ_{H} ppm: 0.90 (6H, d, J=6.2 Hz, 2×CH₃ (Leu)), 1.58 (2H, m, CH₂ (Leu)), 1.61 (1H, m, CH (Leu)), 2.19 (1H, m, CH₂), 2.43 (1H, m, CH₂), 2.57 (1H, m, CH₂), 2.93 (1H, m, CH₂ (AHPA)), 3.02 (1H, m, CH₂), 3.05 (1H, t, CH₂ (AHPA)), 3.65 (3H, s, OCH₃), 3.86 (3H, s, OCH₃), 3.90 (3H, s, OCH₃), 3.94 (3H, s, OCH₃), 4.10 (1H, m, CH (Fmoc)), 4.24 (1H, m, CHOH (AHPA)), 4.24 (2H, d, J=8.3, CH₂ (Fmoc)), 4.25 (H, m, CHNH (Leu)), 4.63 (H, m, CHNH (AHPA)), 5.17 (H, m, CHC═C), 6.12 (1H, d, J=5.0 Hz, C═CH (B-ring)), 6.38 (1H, s, ArH (A-ring)), 6.67 (1H, s, ArH (C-ring)), 6.79 (H, d, J=11.9 Hz, ArH (C-ring)), 6.92 (H, d, J=1.7 Hz, ArH (C-ring)), 7.18 (2H, t, 2×ArH (AHPA)), 7.24 (2H, t, 2×ArH (Fmoc)), 7.29 (3H, t, 3×ArH (AHPA)), 7.40 (2H, t, 2×ArH (Fmoc)), 7.51 (2H, t, 2×ArH (Fmoc)), 7.76 (2H, d, J=8.0 Hz, 2×ArH (Fmoc)).

¹³C NMR (CDCl₃) δ_{c} ppm: 21.5 (CH₂), 22.0 (CH₃ (Leu)), 22.0 (CH₃ (Leu)), 25.4 (CH (Leu)), 36.7 (CH₂ (AHPA)), 39.9 (CH₂), 41.1 (CH₂ (Leu)), 46.6 (CH (Fmoc)), 50.3 (CHNH (AHPA)), 55.9 (OCH₃), 55.63 (OCH₃), 56.2 (CHN (Leu)), 60.6 (CH₂), 60.7 (OCH₃), 61.3 (OCH₃), 67.0 (CH₂ (Fmoc)), 72.7 (CHOH (AHPA)), 73.4 (CHC═C), 109.1 (ArCH (A-ring)), 110.0 (ArCH), 114.7 (ArCH), 118.5 (ArCH), 120.0 (2×ArCH (Fmoc)), 125.2 (2×ArCH (Fmoc)), 125.7 (ArCH (AHPA)), 126.6 (ArCH), 127.1 (2×ArCH (Fmoc)), 127.4 (ArC), 127.7 (2×ArCH (Fmoc)), 128.6 (2×ArCH (AHPA)), 129.3 (2×ArCH (AHPA)), 134.0 (ArC), 135.2 (ArC), 135.9 (ArC), 137.9 (ArC), 140.9 (ArC), 141.3 (2×ArC (Fmoc)), 141.6 (ArC), 143.7 (ArC (Fmoc)), 143.8 (ArC (Fmoc)), 147.4 (ArC), 150.9 (ArC), 151.3 (ArC), 157.1 (NHC═O (Fmoc)), 163.1 (OC═O (AHPA)), 172.5 (OC=0 (Leu)).

νₘₐₓ/cm⁻¹ 3359.2, 2927.7, 1718.0, 1508.7, 1112.2, 740.3

### Synthesis of 48.8—Removal of Fmoc from 48.7 and Synthesis of 49.5—Removal of Fmoc from 49.4

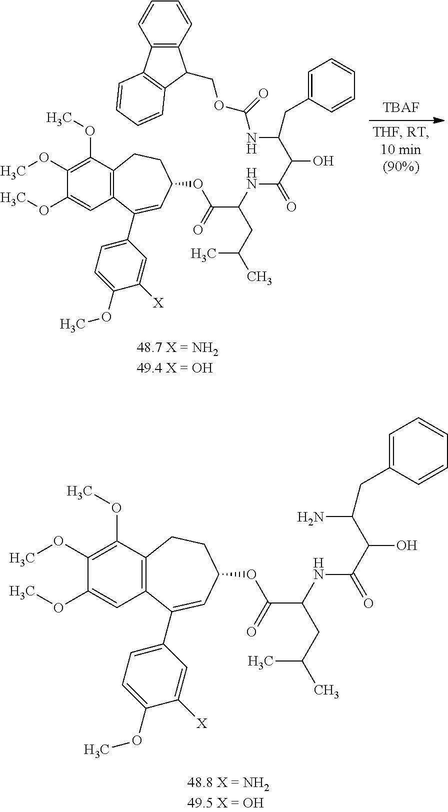

### Synthesis of 48.8—Removal of Fmoc from 48.7

To a stirred solution of 48.7 (30 mg, 0.03 mmol) in anhydrous THF was added 1 M TBAF (0.03 mL, 0.03 mmol) at 0° C. under an atmosphere of nitrogen. The reaction was complete within 10 min and the mixture was applied directly to flash column (stationary phase; silica gel 230-400 mesh, mobile phase; 6:1:1, ethyl acetate/methanol/diethylamine). All homogenous fractions were collected and the solvent was evaporated. The product 48.8 was isolated as an off-white solid (18 mg, 92%).

¹H NMR (CD₃OD) δ_{H} ppm: 0.98 (6H, dd, J=6.1 Hz, 16.3, 2×CH₃ (Leu)), 1.45 (H, m, CH₂ (Leu)), 1.59 (H, m, CH₂ (Leu)), 1.71 (H, m, CH (Leu)), 2.23 (1H, m, CH₂), 2.45 (1H, m, CH₂), 2.55 (1H, m, CH₂ (AHPA)), 2.92 (1H, m, CH₂), 3.02 (1H, m, CH₂ (AHPA)), 3.15 (1H, m, CH₂), 3.67 (3H, s, OCH₃), 3.88 (3H, s, OCH₃), 3.74 (H, m, CHOH (AHPA), 3.90 (3H, s, OCH₃), 4.02 (3H, s, OCH₃), 4.16 (H, d, J=4.0 Hz, CHNH (Leu)), 4.39 (H, dd, J=5.39.8 Hz, CHNH (AHPA), 5.08 (H, qd, J=7.5 Hz, 13.5 Hz, CHC═C), 6.27 (1H, d, J=5.4 Hz, C═CH (B-ring)), 6.35 (1H, s, ArH(A-ring)), 7.24 (H, s, ArH (C-ring)), 7.28 (H, m, ArH (C-ring)), 7.30-7.38 (5H, m, 5×ArH (AHPA)), 7.42 (H, dd, J=2.0 Hz, 7.0 Hz, ArH (C-ring)).

¹³C NMR (CD₃OD) δ_{c} ppm: 20.5 (CH₂), 21.2 (CH₃ (Leu)), 21.8 (CH₃ (Leu)), 24.6 (CH (Leu)), 35.0 (CH₂ (AHPA)), 39.5 (CH₂), 39.6 (CH₂ (Leu)), 51.3 (CHNH (Leu)), 55.0 (OCH₃), 55.7 (OCH₃), 59.8 (OCH₃), 60.8 (OCH₃), 67.5 (CHNH (AHPA)), 68.7 (CHOH (AHPA)), 72.8 (CHO), 108.6 (ArCH (A-ring)), 112.0 (ArCH), 123.13 (ArCH), 127.2 (ArCH (AHPA)), 127.3 (ArCH), 127.5 (ArC), 128.7 (2×ArCH (AHPA)), 129.1 (2×ArCH (AHPA)), 129.4 (ArC), 134.0 (ArC), 134.2 (ArC), 135.3 (ArC), 139.4 (ArC), 142.1 (ArC), 151.0 (ArC), 151.7 (ArC), 152.0 (ArC), 152.4 (ArC), 171.9 (OC═O), 172.3 (OC═O).

νₘₐₓ/cm⁻¹ 2931.4, 1512.8, 1236.4, 1112.9, 702.9

### Synthesis of 49.5—Removal of Fmoc from 49.4

To a stirred solution of 49.4 (700 mg, 0.8 mmol) in anhydrous THF was added 1 M TBAF (0.8 mL, 0.8 mmol) at 0° C. under an atmosphere of nitrogen. After 10 min, the mixture was applied directly to flash column (stationary phase; silica gel 230-400 mesh, mobile phase; 6:1:1, ethyl acetate/methanol/diethylamine). All homogenous fractions were collected and the solvent was evaporated. The product 49.5 was isolated as a golden solid (480 mg, 90%).

¹H NMR (CD₃OD) δ_{H} ppm: 0.97 (6H, dd, J=6.5 Hz, 23.2 Hz, 2×CH₃ (Leu)), 1.56 (1H, m, CH (Leu), 1.66 (1H, m, CH₂ (Leu)), 1.67 (1H, m, CH₂ (Leu)), 2.18 (1H, m, CH₂), 2.46 (1H, m, CH₂), 2.51 (1H, m, CH₂), 2.84 (1H, dd, J=7.7 Hz, 14.1 Hz, CH₂ (AHPA)), 2.98 (1H, m, CH₂), 3.06 (1H, dd, J=7.0 Hz, 13.6 Hz, CH₂ (AHPA)), 3.61 (H, m, CHNH (AHPA), 3.65 (3H, s, OCH₃), 3.87 (6H, s, 2×OCH₃), 3.88 (3H, s, OCH₃), 4.07 (H, d, J=3.4 Hz, CHOH (AHPA), 4.37 (H, dd, J=6.0 Hz, 8.5 Hz, 13.7 Hz, CHNH (Leu)), 5.06 (H, q, CHC═C), 6.10 (1H, d, J=5.4 Hz, C═CH (B-ring)), 6.38 (1H, s, ArH (A-ring)), 6.69 (2H, qd, J=9.1 Hz, 17.4 Hz, ArH (C-ring)), 6.87 (H, d, J=8.6 Hz, ArH (C-ring)), 7.22-7.33 (5H, m, 5×ArH (AHPA)).

¹³C NMR (CD₃OD) δ_{c} ppm: 21.0 (CH₃ (Leu)), 21.5 (CH₂), 22.3 (CH₃ (Leu)), 25.0 (CH (Leu)), 36.5 (CH₂(AHPA)), 39.5 (CH₂ (Leu)), 39.6 (CH₂), 51.6 (CHNH (Leu)), 55.4 (OCH₃), 55.5 (OCH₃), 56.1 (CHNH₂ (AHPA)), 60.3 (OCH₃), 61.1 (OCH₃), 70.2 (CHOH (AHPA)), 73.4 (CHO), 109.3 (ArCH (A-ring)), 111.4 (ArCH (C-ring)), 115.0 (ArCH (C-ring)), 119.4 (ArCH (C-ring)), 125.8 (ArCH (Bring)), 127.3 (ArCH (AHPA)), 127.5 (ArC), 128.7 (2×ArCH (AHPA)), 129.1 (2×ArCH (AHPA)), 129.4 (ArC), 134.0 (ArC), 134.2 (ArC), 135.3 (ArC), 139.4 (ArC), 142.1 (ArC), 151.0 (ArC), 151.7 (ArC), 152.0 (ArC), 152.4 (ArC), 171.9 (OC═O), 172.3 (OC═O).

νₘₐₓ/cm⁻¹ 2958.5, 1664.3, 1509.2, 1120.0, 700.92

### Synthesis of Compounds 50 and 51

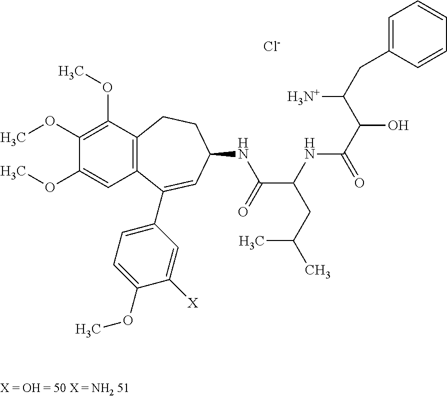

### Synthesis of 4.19—Reduction of Ketone

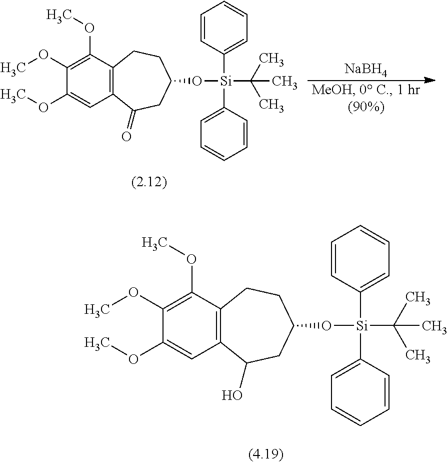

To a stirred solution of the stereoisomerically pure 2.12 (12.7 g, 25.2 mmol) in methanol (100 mL) and THF (55 mL) was added NaBH₄ (1 g, 25.2 mmol) at 0° C. After 1 hr the reaction was quenched by the addition of sat. aq. NaCl (1×100 mL), the organic solvent was removed in vacuo and the product was extracted with diethyl ether (3×100 mL). The combined organic fractions were dried over MgSO₄, filtered and concentrated such that the product could be purified by flash column chromatography (stationary phase; silica gel 230-400 mesh, mobile phase; 4:1, hexane/ethyl acetate). All homogenous fractions were collected and the solvent was evaporated to afford the product 4.19 as a white solid (11.5 g, 90%)

¹H NMR (CDCl₃) δ_{H} ppm: 1.10 (9H, s, major epimer, C(CH₃)₃), 1.18 (9H, s, minor epimer, C(CH₃)₃), 1.39 (H, major epimer, CH₂), 143.2 (H, minor epimer, CH₂), 147.3 (H, major epimer, CH₂), 1.86 (H, minor epimers, CH₂), 3.24 (2H, m, major & minor epimers, CH₂), 3.80 (3H, s, major epimer, OCH₃), 3.81 (3H, s, minor epimer, OCH₃), 3.84 (3H, s, major epimer, OCH₃), 3.85 (3H, s, minor epimer, OCH₃), 3.88 (3H, s, major epimer, OCH₃), 3.89 (3H, s, minor epimer, OCH₃), 4.28 (1H, m, major and minor epimers, CH), 4.71 (1H, m, major epimer, CH), 5.46 (1H, m, minor epimer, CH), 6.82 (1H, s minor epimer, ArH) 6.94 (1H, s, major epimer, ArH), 7.45 (6H, m, major & minor epimers, ArH), 7.80 (4H, m, major & minor epimers, ArH)

¹³C NMR (CDCl₃) δ_{c} ppm: 17.7, 18.0 (C(CH₃)₃), 18.7, 19.0 (CH₂), 26.6, 26.8 (C(CH₃)₃), 34.5 (CH₂), 44.3 (CH₂), 55.5 (OCH₃), 60.3 (OCH₃), 60.6 (OCH₃), 69.9 (CHO), 73.4 (CHOH), 103.3 (ArCH), 125.3 (ArC), 127.2 (4×ArCH), 129.3 (ArCH), 129.4 (ArCH), 133.7 (ArC), 135.4 (4×ArCH), 139.9 (ArC), 140.7 (ArC), 150.4 (ArC), 150.5 (ArC).

νₘₐₓ/cm⁻¹ 3730.0, 2941.5, 1597.3, 1110.0, 697.2

### Synthesis of 4.20—Acetylation

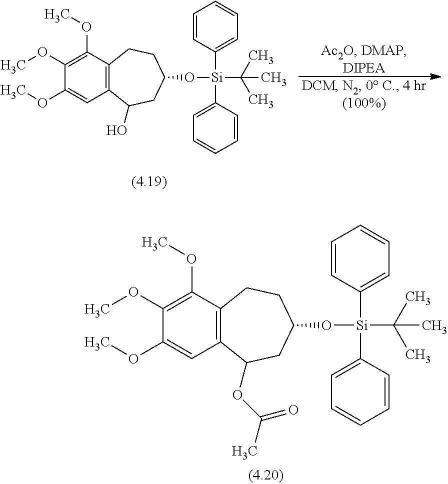

To a stirred solution of 4.19 (9.7 g, 19.1 mmol), DMAP (2.34 g, 19.1 mmol) and N,N-diisopropylethylamine (6.5 mL, 38.3 mmol) in anhydrous DCM (100 mL) was added acetic anhydride (3.6 mL, 38.3 mmol) at 0° C. under an atmosphere of nitrogen. After 4 hr the reaction was quenched by the addition of 2 M aq. HCl (1×100 mL) and the product was extracted with diethyl ether (3×100 mL). The combined organic fractions were dried over MgSO₄, filtered and concentrated to afford a yellow oil. The product was purified by flash column chromatography (stationary phase; silica gel 230-400 mesh, mobile phase; 4:1, hexane/ethyl acetate). All homogenous fractions were collected and the solvent was evaporated to afford the product 4.20 as a colourless oil, (10.5 g, 100%). (Purification by flash column chromatography was only necessary for characterisation of the product, not for progression to the next step in the synthesis).

¹H NMR (CDCl₃) δ_{H} ppm: 1.10 (9H, s, major epimer, C(CH₃)₃), 1.13 (9H, s, minor epimer, C(CH₃)₃), 1.40 (2H, m, major & minor epimers, CH₂), 1.90 (2H, m, major & minor epimers, CH₂), 2.22 (3H, s, major & minor epimers, COCH₃), 2.89 (2H, m, minor epimer, CH₂), 3.17 (2H, m, major epimer, CH₂), 3.84 (3H, s, major epimer, OCH₃), 3.86 (3H, s, minor epimer, OCH₃), 3.90 (6H, s, major and minor epimer, 2×OCH₃), 4.15, (2H, m, major & minor epimers, 2×CH), 6.64 (1H, s, minor epimer, ArH), 6.71 (1H, s, major epimer, ArH), 7.42-7.50 (6H, m, major & minor epimers, 6×ArH), 7.72-7.75 (4H, m, major & minor epimers, 4×ArH)

¹³C NMR (CDCl₃) & ppm: 18.1, 18.7 (C(CH₃)₃), 18.8 (CH₂), 20.7 (COCH₃), 26.6 (C(CH₃)₃), 35.8 (CH₂), 40.9, 43.3 (CH₂), 55.5 (OCH₃), 60.0 (OCH₃), 60.9 (OCH₃), 69.3 (CHO), 72.5 (CH), 102.7 (ArCH), 124.6 (ArC), 127.1 (4×ArCH), 129.2 (2×ArCH), 133.8 (ArC), 135.3 (2×ArCH), 135.4 (2×ArCH), 135.9 (ArC), 140.9 (ArC), 150.4 (ArC), 150.8 (ArC), 169.4 (C═O).

νₘₐₓ/cm⁻¹ 2945.0, 1699.0, 1605.2, 1112.7, 699.5

### Synthesis of 4.21—Removal of Silyl Group

To a stirred solution of 4.20 (9.3 g, 17.0 mmol) in THF (50 mL) was added 1 M TBAF in THF (18.0 mL, 18.0 mmol) at 0° C. After 6 hr the reaction mixture was applied directly to a flash column (stationary phase; silica gel 230-400 mesh, mobile phase; 1:1, hexane/ethyl acetate). All homogenous fractions were collected and the solvent was evaporated to afford the product (5.20) as a colourless oil (5.30 g, 100%).

¹H NMR (CDCl₃) δ_{H} ppm: 1.89 (2H, m, major & minor diasteromers, CH₂), 2.11 (3H, s, minor epimer, COCH₃), 2.19 (3H, s, major epimer, COCH₃), 2.29 (2H, m, major & minor epimers, CH₂), 3.05 (2H, m, major & minor epimers, CH₂), 3.78 (3H, s, minor epimer, OCH₃), 3.79 (3H, s, major epimer, OCH₃), 3.84 (6H, s, minor epimer, 2×OCH₃), 3.85 (6H, s, major epimer, 2×OCH₃), 4.05 (2H, m, major & minor epimers, 2×CH), 6.66 (1H, s, minor diasteroemer, ArH), 6.70 (1H, s, major epimer, ArH).

¹³C NMR (CDCl₃) δ_{c} ppm: 18.4, 18.8 (CH₂), 20.8 (COCH₃), 35.3 (CH₂), 40.4, 42.4 (CH₂), 55.5 (OCH₃), 59.9, 60.3 (OCH₃), 60.3, 60.9 (OCH₃), 68.2 (CH), 70.1, 70.9 (CH), 103.3 (ArCH), 124.9, 126.7 (ArC), 135.2 (ArC), 140.8, 141.3 (ArC), 150.4, 150.6 (ArC), 150.8 (ArC), 169.4 (C═O)

νₘₐₓ/cm⁻¹ 3296.2, 2930.1, 1736.0, 1601.5, 1115.4

### Synthesis of 4.23—Mesylation and Azide Substitution

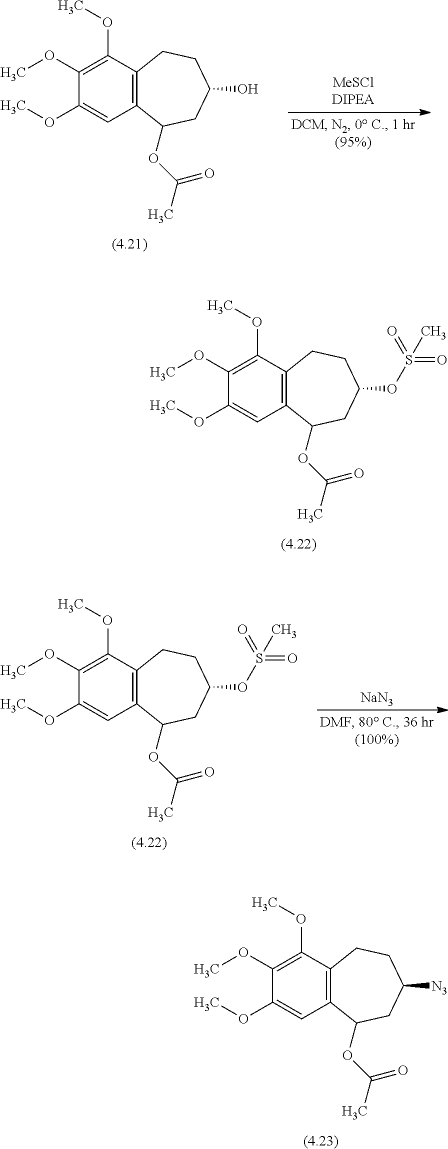

To a stirred solution of 4.21 (5.0 g, 16.1 mmol) in anhydrous DCM (50 mL) was added methanesulphonyl chloride (2.37 mL, 30.6 mmol) followed by N,N diisopropylethylamine (4.4 mL, 25.8 mmol) at 0° C. under dry reaction conditions. After one hour the reaction was quenched by the addition of water (1×50 mL) and the product was extracted into diethyl ether (3×50 mL). The combined organic fractions were dried over MgSO₄, filtered, concentrated and purified by flash column chromatography (stationary phase; silica gel 230-400 mesh, mobile phase; 1:1, hexane/ethyl acetate). All homogenous fractions were collected and the solvent was evaporated to afford the product 4.22 as a white solid (5.90 g, 95%). Without further characterisation, the mesylate 4.22 (5.90 g, 15.3 mmol) was dissolved in DMF (20 mL) at room temperature. Sodium azide (17.4 g, 26.8 mmol) was added to the stirred solution. The reaction mixture was heated to 80° C. After 36 hr the reaction was quenched by the addition of water (1×100 mL) and the product was extracted into diethyl ether (3×60 mL). The combined etheral fractions were dried over MgSO₄, filtered, concentrated to and purified by flash column chromatography (stationary phase; silica gel 230-400 mesh, mobile phase; 3:1, hexane/ethyl acetate). All homogenous fractions were collected and the solvent was evaporated to afford the product 4.23 as a colourless oil (4.5 g, 100%).

4.23 ¹H NMR (CDCl₃) δ_{H} ppm: 1.65 (2H, m, minor epimer, CH₂), 1.84 (2H, m, major epimer, CH₂), 2.12 (3H, s, major epimer, C═OCH₃), 2.22 (3H, s, minor epimer, C═OCH₃), 2.30 (1H, m, major & minor epimers, CH₂), 2.37 (1H, m, major & minor epimers, CH₂), 2.77 (2H, m, major & minor epimers, CH₂), 3.15 (2H, m, major & minor epimers, CH₂), 3.80 (3H, major epimer OCH₃), 3.82 (3H, s, minor epimer, OCH₃), 3.85 (6H, s, minor epimer, 2×OCH₃), 3.87 (6H, s, major epimer, 2×OCH₃), 3.94 (1H, m, major & minor epimers, CH), 6.86 (1H, d, J=10.4 Hz, major epimer, ArH), 6.01 (1H, d, J=10.4 Hz, minor epimer, ArH).

¹³C NMR (CDCl₃) δ_{c} ppm: 19.6, 19.9 (CH₂), 20.7 (COCH₃), 31.9, 32.5 (CH₂), 36.9, 39.2 (CH₂), 55.5 (OCH₃), 58.9 (CHN₃), 60.4 (OCH₃), 60.9 (OCH₃), 69.8 (CH), 103.0 (ArCH), 124.1, 126.5 (ArC), 134.0, 134.9 (ArC), 140.8, 141.7 (ArC), 150.6 (ArC), 151.0 (ArC), 169.2 (C═O).

νₘₐₓ/cm⁻¹ 2941.0, 1993.4, 1739.1, 1589.3, 1230.1, 1111.8

### Synthesis of 4.25—De-Acetylation and Oxidation

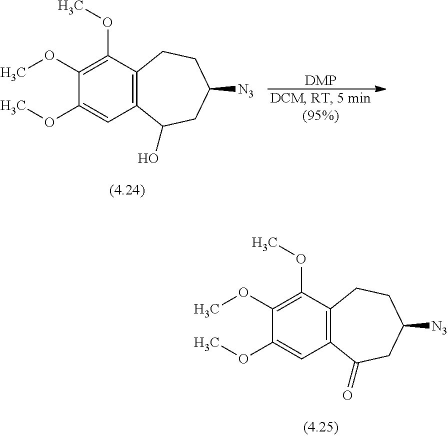

To a stirred solution of 4.23 (5.0 g, 14.9 mmol) in methanol (20 mL) was added 2.5 M aq. NaOH (20 mL) at 0° C. After 10 min the flask was removed from the ice and brought to room temperature. After an hour the reaction mixture was deemed complete by TLC and quenched with sat. aq. NaCl solution (1×50 mL). The product was extracted with diethyl ether (3×50 mL). The combined etheral fractions were dried over MgSO₄, filtered and concentrated under vacuum. The product 4.24 was not purified by flash column chromatography but was redissolved in DCM (20 mL) and Dess Martin periodinane (9.5 g, 22.4 mmol) was added to the stirred solution at room temperature. The oxidation reaction was complete within 5 min and an extraction between 0.5 M NaOH (100 mL) and diethyl ether (3×50 mL) separated the product from the precipitated iodinane which dissolved in aq. NaOH. The organic extracts were combined, dried over magnesium sulphate and filtered and the product was purified by flash column chromatography (stationary phase; silica gel 230-400 mesh, mobile phase; 3:1, hexane/ethyl acetate). All homogenous fractions were collected and the solvent was evaporated to afford the product 4.25 as a colourless oil (4.12 g, 95%).

¹H NMR (CDCl₃) δ_{H} ppm: 1.90 (2H, m, CH₂), 2.99 (2H, m, CH₂), 3.05 (2H, m, CH₂), 3.80 (3H, s, OCH₃), 3.87 (6H, s, 2×OCH₃), 4.00 (1H, m, CHN₃), 7.10 (1H, s, ArH)

¹³C NMR (CDCl₃) δ_{c} ppm: 20.6 (CH₂), 33.2 (CH₂), 49.2 (CH₂), 55.8 (OCH₃), 56.5 (CHN₃), 60.5 (OCH₃), 60.7 (OCH₃), 107.2 (ArCH), 130.2 (ArC), 133.3 (ArC), 145.2 (ArC), 150.6 (ArC), 151.3 (ArC), 199.1 (C═O)

νₘₐₓ/cm⁻¹ 2911.2, 1989.4, 1590.7, 1492.5, 1074.1

### Synthesis of 4.26—Azide Reduction and N-Boc Protection

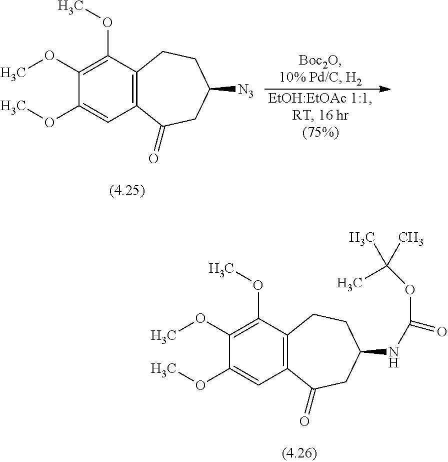 [SMILES: COc1cc2c(c(OC)c1OC)CC[C@@H](N=[N+]=[N-])CC2=O ; "Boc2O,10% Pd/C, H2" ; "EtOH:EtOAc 1:1, RT, 16 hr(75%)" ; COc1cc2c(c(OC)c1OC)CC[C@@H](NC(=O)OC(C)(C)C)CC2=O]

To a stirred solution of 4.25 (2.91 g, 10.0 mmol) in a 1:1 mixture of ethanol and ethyl acetate (20 mL) was added di-tert-butyl dicarbonate (4.36 g, 20.0 mmol) and 10% Pd/C (catalytic amount). The mixture was stirred at room temperature under an atmosphere of hydrogen (balloon). After 16 hr the reaction was deemed complete by TLC and the Pd/C was removed from the reaction mixture by filtration, washing with DCM (200 mL). The solvent was removed from the flask under vacuum and the resulting residue was purified by flash column chromatography (stationary phase; silica gel 230-400 mesh, mobile phase; 2:1, hexane/ethyl acetate). All homogenous fractions were collected and the solvent was evaporated to afford 4.26 as a yellow solid (2.74 g, 75%).

¹H NMR (CDCl₃) δ_{H} ppm: 1.39 (9H, s C(CH₃)₃), 1.54 (1H, s, CH₂), 2.26 (1H, s, CH₂), 2.74-3.03 (4H, m, 2×CH₂), 3.79 (3H, s, OCH₃), 3.81 (3H, s, OCH₃), 3.87 (3H, s, OCH₃), 4.05 (1H, m, CH), 4.84 (1H, s, NH), 7.04 (1H, s, ArH)

¹³C NMR (CDCl₃) δ_{c} ppm: 22.4 (CH₂), 28.6 (C(CH₃)₃), 32.8 (CH₂), 46.6 (CH), 48.3 (CH₂), 56.2 (OCH₃), 60.9 (OCH₃), 61.2 (OCH₃), 79.5 (C(CH₃)₃), 107.4 (ArCH), 129.4 (ArC), 134.0 (ArC), 145.8 (ArC), 151.1 (ArC), 151.8 (ArC), 155.1 (C═O), 200.8 (C═O)

νₘₐₓ/cm⁻¹ 3336.7, 2998.4, 1718.2, 1406.2, 1140.2

### Synthesis of 4.27—Triflation

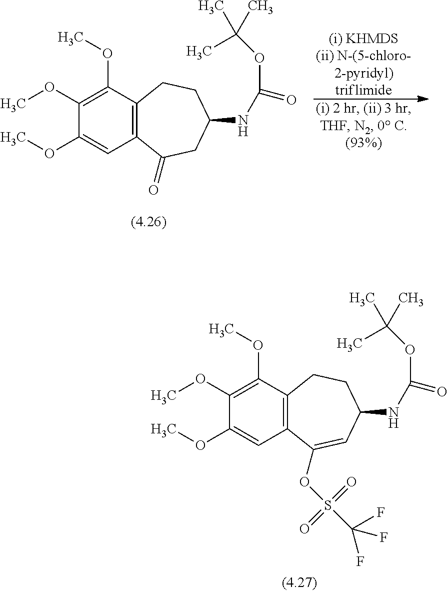 [SMILES: COc1cc2c(c(OC)c1OC)CC[C@@H](NC(=O)OC(C)(C)C)CC2=O ; "(i) 2 hr, (ii) 3 hr,THF, N2, 0° C.(93%)" ; "(i) KHMDS(ii) N-(5-chloro-2-pyridyl)triflimide" ; COc1cc2c(c(OC)c1OC)CC[C@@H](NC(=O)OC(C)(C)C)C=C2OS(=O)(=O)C(F)(F)F]

To a dry three-necked round bottom flask containing 4.26 (2.1 g, 0.05 mmol) dissolved in anhydrous THF (7 mL) was added KHMDS (0.5 M in toluene) (18.4 mL 9.2 mmol) under dry reaction conditions at 0° C. The reaction was allowed to stir at this temperature for 2 hr after which time a solution of N,N-bis-(trifluoromethylsulfonyl)amino-5-chloropyridine (6.77 mg, 17.3 mmol) in dry THF (10 mL) was added. The reaction was left stirring for an additional 3 hr at this temperature after which time it was quenched by the addition of water (1×50 mL) and extracted with diethyl ether (3×50 mL). The combined organic fractions were dried over MgSO₄, filtered and concentrated under vacuum. The residue was purified by flash column chromatography (stationary phase; silica gel 230-400 mesh, mobile phase; 6:1, hexane/ethyl acetate). All homogenous fractions were collected and the solvent was evaporated to yield 4.27 as a colourless oil (2.67 g, 93%).

¹H NMR (CDCl₃) δ_{H} ppm: 1.46 (9H, s C(CH₃)₃), 2.08 (2H, m, CH₂), 2.96 (2H, m, CH₂), 3.46 (2H, m, CH₂), 3.87 (6H, s, 2×OCH₃), 3.93 (3H, s, OCH₃), 4.19 (1H, m, CH), 4.60 (1H, m, NH), 5.22 (1H, d, J=4.1 Hz, C═CH), 6.79 (1H, s, ArH)

¹³C NMR (CDCl₃) δ_{c} ppm: 21.4 (CH₂), 28.3 (C(CH₃)₃), 39.2 (CH₂), 46.3 (CH), 55.9 (OCH₃), 60.4 (OCH₃), 61.6 (OCH₃), 80.0 (C(CH₃)₃), 105.5 (ArCH), 124.6 (C═CH), 127.2 (ArC), 130.3 (ArC), 143.5 (ArC), 144.1 (ArC), 150.6 (ArC), 151.4 (ArC), 152.6 (C═O)

¹⁹F NMR (CDCl₃) δ_{c} ppm: −74.39 (CF₃)

νₘₐₓ/cm⁻¹ 3339.2, 2910.5, 1700.1, 1115.4, 773.4.

### Synthesis of 4.29—Suzuki Coupling (Phenol) and

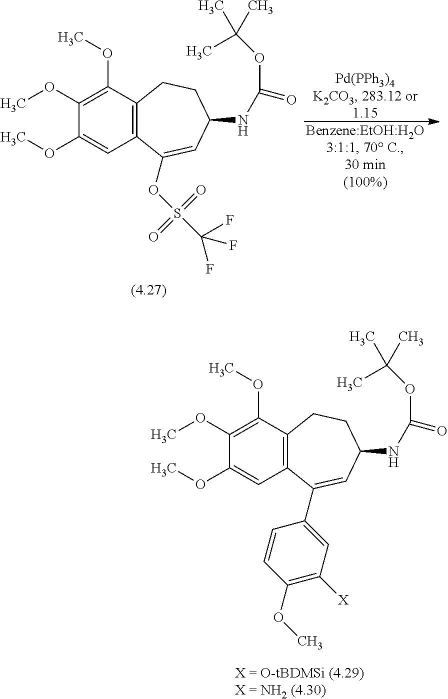

### Synthesis of 4.29—Suzuki Coupling (Phenol)

The triflate 4.27 (120 mg, 0.24 mmol), the boronic acid 1.15 (70 mg, 0.24 mmol), K₂CO₃ (90 mg, 0.65 mmol), and Pd(Ph₃)₄ (2 mg, 0.01 mmol) were dissolved in a mixture of benzene, ethanol and water (3:1:1. 1.6 mL). The reaction was refluxed at 70° C. for 30 min, after which time it was quenched by the addition of water (1×20 mL) and the product was extracted into diethyl ether (3×30 mL). The organic fractions were combined, dried over MgSO₄, filtered and concentrated in vacuo. The resulting residue was purified by flash column chromatography (stationary phase; silica gel 230-400 mesh, mobile phase; 4:1, hexane/ethyl acetate). All homogenous fractions were collected and the solvent was evaporated to afford 4.29 as a colourless oil (140 mg, 100%).

¹H NMR (CDCl₃) δ_{H} ppm: 0.15 (3H, s, Si(CH₃)), 0.16 (3H, s, Si(CH₃)), 1.0 (9H, s C(CH₃)₃ (tbdms)), 1.45 (9H, s, C(CH₃)₃ (Boc)), 1.93 (1H, m, CH₂), 2.31 (1H, m, CH₂), 2.48 (1H, m, CH₂), 3.06 (1H, m, CH₂), 3.68 (3H, s, OCH₃), 3.83 (3H, s, OCH₃), 3.93 (6H, s, 2×OCH₃), 4.04 (1H, m, CHNH), 6.02 (1H, d, J=5.5 Hz, C═CH), 6.36 (1H, s, ArH {A-ring}), 6.80 (3H, m, 3×ArH {C-ring})

¹³C NMR (CDCl₃) δ_{c} ppm: −5.1 (2×Si(CH₃)₂), 18.1 (C(CH₃)₃ (tbdms)), 21.8 (CH₂), 25.4 (3×C(CH₃)₃ (tbdms)), 28.1 (3×C(CH₃)₃ (Boc)), 41.7 (CH₂), 48.7 (CHNH), 55.1 (OCH₃), 55.4 (OCH₃), 60.6 (OCH₃), 61.1 (OCH₃), 78.9 (C(CH₃)₃ (Boc)), 108.7 (ArCH), 111.3 (ArCH), 120.2 (ArCH), 121.1 (ArCH), 126.8 (ArC), 129.0 (C═CH), 133.8 (2×ArC), 134.8 (ArC), 141.0 (ArC), 144.1 (ArC), 150.1 (ArC), 150.3 (ArC), 150.8 (ArC), 154.8 (C═O)

νₘₐₓ/cm⁻¹ 3402.1, 2932.6, 1516.8, 1199.7

### Synthesis of 4.30—Suzuki Coupling (Aniline)

The triflate 4.27 (2.0 g, 4.02 mmol), the boronic ester 28.12 (1.0 g, 4.02 mmol), K₂CO₃ (1.5 g, 10.85 mmol), and Pd(Ph₃)₄ (30 mg, 0.20 mmol) were dissolved in a mixture of benzene, ethanol and water (3:1:1, 30 mL). The reaction was refluxed at 70° C. for 30 min, after which time it was quenched by the addition of water (1×30 mL) and the product was extracted with diethyl ether (3×50 mL). The organic fractions were combined, dried over MgSO₄, filtered and concentrated in vacuo. The resulting residue was purified by flash column chromatography (stationary phase; silica gel 230-400 mesh, mobile phase; 3:1, hexane/ethyl acetate). All homogenous fractions were collected and the solvent was evaporated to afford 4.30 as a colourless oil (1.90 g, 100%).

¹H NMR (CDCl₃) δ_{H} ppm: 1.45 (9H, s, C(CH₃)₃ (Boc)), 1.92 (1H, m, CH₂), 2.30 (1H, m, CH₂), 2.44 (1H, m, CH₂), 3.03 (1H, dd, J=7.2 Hz, 13.6 Hz, CH₂), 3.58 (3H, s, OCH₃), 3.85 (3H, s, OCH₃), 3.90 (H, s, OCH₃), 3.91 (H, s, OCH₃), 4.02 (1H, m, CHNH), 6.00 (1H, d, J=5.5 Hz, C═CH), 6.38 (1H, s, ArH {A-ring}), 6.70 (3H, m, 3×ArH {C-ring})

¹³C NMR (CDCl₃) δ_{c} ppm: 21.8 (CH₂), 28.0 (3×C(CH₃)₃ (Boc)), 41.5 (CH₂), 48.7 (CHNH), 55.0 (OCH₃), 55.6 (OCH₃), 60.3 (OCH₃), 61.2 (OCH₃), 79.0 (C(CH₃)₃ (Boc)), 108.7 (ArCH), 109.6 (ArCH), 114.4 (ArCH), 118.0 (ArCH), 126.9 (ArC), 129.0 (C═CH), 133.8 (ArC), 135.0 (ArC), 135.4 (ArC), 139.8 (ArC), 140.8 (ArC), 146.4 (ArC), 150.1 (ArC), 150.8 (ArC), 154.8 (C═O)

νₘₐₓ/cm⁻¹ 3371.2, 2911.5, 1711.1, 1510.7, 760.0

### Synthesis of 4.31—Fmoc Protection of Aniline

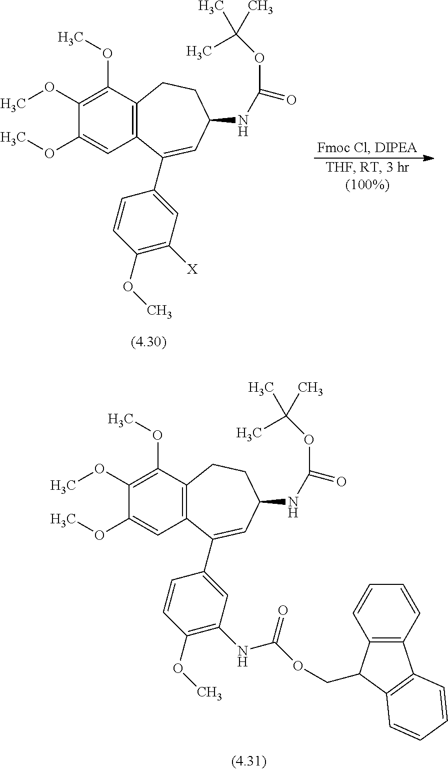

To a solution of 4.30 (2.40 g, 5.1 mmol) and DIPEA (1.7 mL, 10.2 mmol) in toluene (10 mL) was added Fmoc chloride (2.64 g, 10.2 mmol) in THF (10 mL). After 3 hr at room temperature the solvent was removed under vacuum and the product was isolated by flash column chromatography (stationary phase; silica gel 230-400 mesh, mobile phase; 4:1, hexane/ethyl acetate). All homogenous fractions were collected and the solvent was evaporated to afford the product as a white solid (3.45 g, 100%).

¹H NMR (CDCl₃) δ_{H} ppm: 1.46 (9H, s, C(CH₃)₃ (Boc)), 1.95 (1H, m, CH₂), 2.33 (1H, m, CH₂), 2.51 (1H, m, CH₂), 3.07 (1H, dd, J=6.2 Hz, 12.9 Hz, CH₂), 3.71 (3H, s, OCH₃), 3.93 (9H, s, 3×OCH₃), 4.09 (1H, m, CHNH), 4.32 (1H, t, CH (Fmoc)), 4.50 (2H, m, CH₂ (Fmoc)), 6.11 (1H, d, J=5.5 Hz, C═CH), 6.39 (1H, s, ArH {A-ring}), 6.85 (2H, dd, J=8.6 Hz, 30.5 Hz, 2×ArH {C-ring}), 7.38 (5H, m, 4×ArH (Fmoc), 1×ArH {C-ring}), 7.66 (2H, d, J=8.8 Hz, 2×ArH (Fmoc)), 7.81 (2H, d, J=7.7 Hz, 2×ArH (Fmoc)).

¹³C NMR (CDCl₃) δ_{c} ppm: 21.8 (CH₂), 28.0 (3×C(CH₃)₃ (Boc)), 41.5 (CH₂), 47.2 (CH (Fmoc)), 48.7 (CHNH), 55.0 (OCH₃), 55.6 (OCH₃), 60.3 (OCH₃), 61.2 (OCH₃), 67.1 (CH₂ (Fmoc)), 79.0 (C(CH₃)₃ (Boc)), 108.7 (ArCH), 109.6 (ArCH), 114.4 (ArCH), 118.0 (ArCH), 125.1 (2×ArCH (Fmoc)), 126.9 (ArC), 127.1 (2×ArCH (Fmoc)), 127.8 (2×ArCH (Fmoc)), 128.2 (2×ArC (Fmoc)), 129.0 (C═CH), 132.2 (2×ArC (Fmoc)), 133.8 (ArC), 135.0 (ArC), 135.4 (ArC), 139.8 (ArC), 140.8 (ArC), 141.3 (2×ArC (Fmoc)), 146.4 (ArC), 150.1 (ArC), 150.8 (ArC), 153.4 (C═O (Fmoc)), 154.8 (C═O(Boc)).

νₘₐₓ/cm⁻¹ 3363.4, 2931.2, 1511.9, 1234.6, 1111.4, 1027.1

### Synthesis of 4.32—Removal of N-Boc Group (Phenol)

Through a stirred solution of 4.29 (90 mg, 0.15 mmol) in DCM (1 mL) was bubbled HCl gas. The reaction was complete after 40 min and the solvent was removed in vacuo. The product was isolated as a colourless oil (70 mg, 100%).

¹H NMR (CDCl₃) δ_{H} ppm: 0.15 (3H, s, Si(CH₃)), 0.16 (3H, s, Si(CH₃)), 1.0 (9H, s C(CH₃)₃ (tbdms)), 2.19 (1H, m, CH₂), 2.38 (1H, m, CH₂), 2.50 (1H, m, CH₂), 3.17 (1H, dd, J=5.9 Hz, 11.7 Hz, CH₂), 3.50 (1H, m, CHNH₂), 3.69 (3H, s, OCH₃), 3.85 (3H, s, OCH₃), 3.89 (3H, s, OCH₃), 3.93 (3H, s, OCH₃), 6.015 (1H, d, J=6.0 Hz, C═CH), 6.43 (1H, s, ArH {A-ring}), 6.80 (H, d, J=2.1, ArH {C-ring}), 6.94 (2H, dd, J=8.7 Hz, 15.5 Hz, 2×ArH {C-ring}).

¹³C NMR (CDCl₃) δ_{c} ppm: −5.8 (2×Si(CH₃)₂), 17.8 (C(CH₃)₃), 21.7 (CH₂), 25.0 (3×C(CH₃)₃), 40.1 (CH₂), 49.0 (CHNH), 54.4 (OCH₃), 55.4 (OCH₃), 60.0 (OCH₃), 60.8 (OCH₃), 108.8 (ArCH), 111.5 (ArCH), 120.4 (ArCH), 121.4 (ArCH), 123.5 (C═CH), 126.9 (ArC), 133.2 (ArC), 134.7 (ArC), 142.0 (ArC), 142.4 (ArC), 144.7 (ArC), 150.9 (ArC), 151.2 (ArC), 151.8 (ArC).

νₘₐₓ/cm⁻¹ 3400.3, 2937.0, 1593.9, 1509.4, 1252.5, 1116.7

### Synthesis of 4.33—Removal of N-Boc Group

Through a stirred solution of 4.31 (2.34 g, 3.38 mmol) in DCM (8 mL) was bubbled HCl gas. The reaction was complete after one hour and the solvent was removed in vacuo. The product was isolated as a white solid (2.0 g, 100%).

¹H NMR (CDCl₃) δ_{H} ppm: 2.29 (2H, m, CH₂), 2.53 (1H, m, CH₂), 3.05 (1H, m, CH₂), 3.65 (3H, s, OCH₃), 3.88 (6H, s, 2×OCH₃), 3.92 (3H, s, OCH₃), 4.23 (1H, m, CHNH₂), 4.46 (3H, t, CH (Fmoc), CH₂ (Fmoc)), 6.27 (1H, d, J=4.5 Hz, C═CH), 6.35 (1H, s, ArH {A-ring}), 6.68 (3H, dd, J=8.0 Hz, 51.5 Hz, 3×ArH {C-ring}), 7.30 (2H, m, 2×ArH (Fmoc), 7.41 (2H, t, 2×ArH (Fmoc)), 7.58 (2H, t, 2×ArH (Fmoc)), 7.77 (2H, d, J=7.1 Hz, 2×ArH (Fmoc)), 8.03 (1H, br, NH), 8.47 (2H, br, NH₂).

¹³C NMR (CDCl₃) δ_{c} ppm: 21.3 (CH₂), 39.5 (CH₂), 46.7 (CH (Fmoc)), 49.4 (CHNH), 55.3 (OCH₃), 55.6 (OCH₃), 60.5 (OCH₃), 61.2 (OCH₃), 66.8 (CH₂ (Fmoc)), 108.4 (ArCH), 109.2 (ArCH), 119.5 (2×ArCH (Fmoc)), 121.6 (ArCH), 123.1 (ArCH), 124.5 (2×ArCH (Fmoc)), 126.2 (ArC), 126.6 (ArC), 126.8 (2×ArCH (Fmoc)), 127.4 (2×ArCH (Fmoc)), 128.2 (2×ArC (Fmoc)), 132.6 (ArC), 133.8 (ArC), 140.8 (2×ArC (Fmoc)), 141.5 (ArC), 142.4 (ArC), 143.1 (2×ArC (Fmoc)), 147.5 (ArC), 150.5 (ArC), 151.2 (ArC), 153.0 (C═O (Fmoc)).

νₘₐₓ/cm⁻¹ 2939.6, 1671.7, 1530.4, 1200.1, 1114.1, 734.8

### Synthesis of 4.34—Coupling of N-Boc Bestatin (Protected Phenol) and Synthesis of 4.35—Coupling N-Boc Bestatin (Protected Aniline)

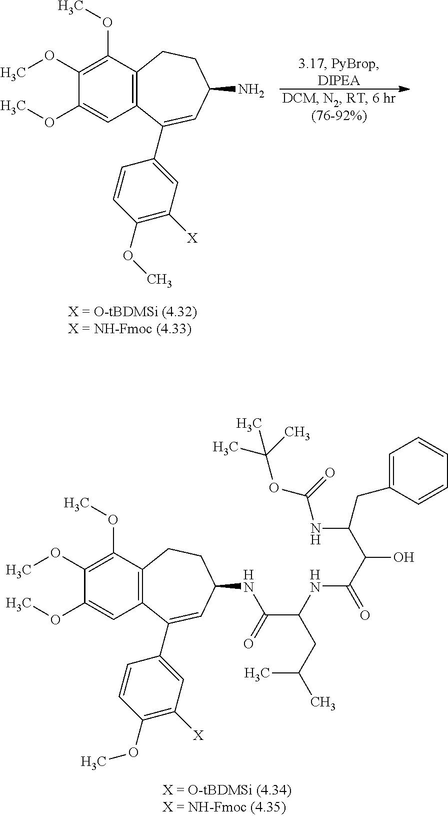

### Synthesis of 4.34—Coupling of N-Boc Bestatin (Protected Phenol)

To a stirred solution of 4.32 (90 mg, 0.19 mmol) in dry DCM (2 mL) was added a solution of 3.17 (76 mg, 0.19 mmol), PyBrop (142 mg, 0.30 mmol) and DIPEA (0.06 mL, 0.38 mmol) in dry DCM (3 mL) at 0° C. The reaction temperature was allowed to increase to room temperature and left stirring for 6 hr. The reaction was then quenched by the addition of 1 M HCl (5 mL) and extracted into diethyl ether (3×10 mL). The organic fractions were combined, dried over MgSO₄, filtered and concentrated in vacuo. The product was isolated by flash column chromatography (stationary phase; silica gel 230-400 mesh, mobile phase; 2:1, hexane:ethyl acetate). All homogenous fractions were collected and the solvent was evaporated to afford the product as a light yellow solid (150 mg, 92%).

¹H NMR (CDCl₃) δ_{H} ppm: 0.13 (3H, s, Si(CH₃)), 0.14 (3H, s, Si(CH₃)), 0.91 (6H, dd, J=5.0 Hz, 15.0 Hz, 2×CH₃ (Leu)), 0.98 (9H, s C(CH₃)₃ (tbdms)), 1.40 (9H, s, C(CH₃)₃ (Boc)), 1.59 (2H, d, J=10.0 Hz, CH, CH₂ (Leu)), 1.63 (2H, d, J=10.0 Hz, CH₂ (Leu), CH₂), 2.44 (2H, m, CH₂), 3.02 (2H, m, CH₂ (AHPA bzl)), 3.15 (1H, m, CH₂), 3.67 (3H, s, OCH₃), 3.83 (3H, s, OCH₃), 3.91 (3H, s, OCH₃), 3.93 (3H, s, OCH₃), 4.02 (1H, m, CHOH (AHPA)), 4.33 (1H, m, CHNH (Leu)), 4.43 (1H, t, CHNH (AHPA)), 5.05 (1H, d, J=8.8 Hz, CHC═C), 6.02 (1H, d, J=6.5 Hz, C═CH), 6.34 (1H, s, ArH {A-ring}), 6.69-6.91 (3H, m, 3×ArH {C-ring}), 7.20-7.32 (5H, m, ArH (AHPA)).

¹³C NMR (CDCl₃) δ_{c} ppm: −5.0 (2×Si(CH₃)₂), 18.1 (C(CH₃)₃ (tbdms)), 21.1 (CH₃ (Leu)), 21.7 (CH₂), 22.8 (CH₃ (Leu)), 24.5 (CH(CH₃)₂ (Leu)), 25.3 (3×C(CH₃)₃ (tbdms)), 27.8 (3×C(CH₃)₃ (Boc)), 40.5 (CH₂ (AHPA)), 41.1 (CH₂ (Leu), 41.2 (CH₂), 47.6 (CHNH (Leu)), 51.1 (CHNH (AHPA)), 55.0 (OCH₃), 55.3 (CHOH (AHPA)), 55.5 (OCH₃), 60.5 (OCH₃), 61.2 (OCH₃), 74.1 (CHC═C), 80.1 (C(CH₃)₃ (Boc)), 108.5 (ArCH), 111.4 (ArCH), 120.4 (ArCH), 121.0 (ArCH), 126.2 (ArCH), 127.2 (ArCH), 127.3 (ArC), 128.1 (2×ArCH), 128.8 (2×ArCH), 133.6 (ArC), 134.7 (ArC), 137.6 (ArC), 140.5 (ArC), 141.1 (ArC), 144.1 (ArC), 150.2 (ArC), 150.3 (ArC), 150.8 (ArC), 157.0 (NHC═O (Boc)), 170.4 (NHC═O (AHPA)), 172.6 (NHC═O (Leu)).

νₘₐₓ/cm⁻¹ 3311.8, 2920.8, 1651.2, 1500.7, 1359.0, 1160.4

### Synthesis of 4.35—Coupling N-Boc Bestatin (Protected Aniline)

To a stirred solution of 4.33 (680 mg, 1.2 mmol) in dry DCM (5 mL) was added a solution of tBoc bestatin (470 mg, 1.2 mmol), PyBrop (860 mg, 1.8 mmol) and DIPEA (0.30 mL, 1.8 mmol) in dry DCM (5 mL) at 0° C. The reaction temperature was allowed to increase to room temperature and left stirring for 6 hr. The reaction was then quenched by the addition of 1 M HCl (5 mL) and extracted into diethyl ether (3×10 mL). The organic fractions were combined, dried over MgSO₄, filtered and concentrated in vacuo. The product was isolated by flash column chromatography (stationary phase; silica gel 230-400 mesh, mobile phase; 2:1, hexane:ethyl acetate). All homogenous fractions were collected and the solvent was evaporated to afford the product as a yellow solid (860 mg, 76%).

¹H NMR (CDCl₃) δ_{H} ppm: 0.89 (6H, dd, J=5.9 Hz, 14.7 Hz, 2×CH₃ (Leu)), 1.33 (9H, s, C(CH₃)₃ (Boc)), 1.59 (3H, m, CH₂ (Leu), CH (Leu)), 2.00 (H, m, CH₂), 2.31 (H, m, CH₂), 2.42 (2H, m, CH₂), 2.83 (H, d, J=8.0 Hz, CH₂ (AHPA bzl)), 3.00 (H, dd, J=4.7 Hz, 12.0 Hz, CH₂ (AHPA bzl)), 3.64 (3H, s, OCH₃), 3.89 (3H, s, OCH₃), 3.90 (6H, s, 2×OCH₃), 4.15 (1H, m, CH (Fmoc), 4.29 (2H, m, CHOH (AHPA), CHNH (Leu)), 4.47 (3H, m, CH₂ (Fmoc), CHNH (AHPA)), 5.12 (1H, d, J=8.5 Hz, CHC═C), 6.01 (1H, d, J=5.5 Hz, C═CH), 6.34 (1H, s, ArH {A-ring}), 6.78 (H, m, ArH {C-ring}), 7.12-7.21 (5H, m, ArH (AHPA)), 7.25-7.45 (6H, m, 4×ArH (Fmoc), 2×ArH {C-ring}), 7.63 (2H, t, 2×ArH (Fmoc)), 7.77 (2H, d, J=7.5 Hz, 2×ArH (Fmoc)).

¹³C NMR (CDCl₃) δ_{c} ppm: 21.9 (CH₃ (Leu)), 21.4 (CH₂), 23.1 (CH₃ (Leu)), 24.7 (CH(CH₃)₂ (Leu)), 28.2 (3×C(CH₃)₃ (Boc)), 37.4 (CH₂ (AHPA)), 40.4 (CH₂), 41.4 (CH₂ (Leu)), 47.0 (CH (Fmoc)), 48.0 (CHNH (AHPA)), 51.2 (CHNH (Leu)), 54.6 (CHOH (AHPA)), 55.8 (OCH₃), 56.0 (OCH₃), 60.8 (OCH₃), 61.5 (OCH₃), 67.4 (CH₂ (Fmoc)), 72.7 (CHC═C), 79.6 (C(CH₃)₃ (Boc)), 109.3 (2×ArCH), 109.6 (2×ArCH), 120.1 (2×ArCH (Fmoc)), 123.4 (ArCH), 125.2 (2×ArCH (Fmoc)), 126.4 (ArCH), 126.9 (ArC), 127.2 (2×ArCH (Fmoc)), 127.7 (ArC), 127.9 (2×ArCH (AHPA)), 128.4 (2×ArCH (Fmoc)), 129.3 (2×ArCH (AHPA)), 134.0 (ArC), 134.9 (ArC), 138.0 (ArC), 140.4 (ArC), 141.3 (ArC), 141.6 (ArC), 143.6 (ArC), 147.5 (ArC), 150.9 (ArC), 151.3 (ArC), 153.7 (ArC), 156.1 (ArC), 171.33 (C═O (Fmoc), C═O (Boc)), 173.1 (2×C═O (Bestatin)).

νₘₐₓ/cm⁻¹ 3319.8, 2917.8, 1666.3, 1508.8, 1359.0, 1154.2, 768.93

### Synthesis of 4.36—Removal of silyl group

To a stirred solution of 4.34 (30 mg, 0.03 mmol) in THF (1 mL) was added 1 M TBAF (0.03 mL, 0.03 mmol) at 0° C. After 40 min the reaction volume was loaded directly onto a flash column (stationary phase; silica gel 230-400 mesh, mobile phase; 1:1, hexane/ethyl acetate). All homogenous fractions were collected and the solvent was evaporated to afford the product as a colourless oil (22 mg, 100%).

¹H NMR (CDCl₃) δ_{H} ppm: 0.92 (6H, dd, J=5.4 Hz, 16.9 Hz, 2×CH₃ (Leu)), 1.40 (9H, s, C(CH₃)₃ (Boc)), 1.59 (3H, m, CH₂ (Leu), CH (Leu)), 1.96 (1H, dd, J=10.1 Hz, 17.6 Hz, CH₂ (AHPA bzl)), 2.30 (2H, m, CH₂), 2.44 (1H, m, CH₂ (AHPA bzl)), 2.98 (2H, dd, J=5.1 Hz, 12.7 Hz, CH₂), 3.64 (3H, s, OCH₃), 3.90 (6H, s, OCH₃), 3.92 (3H, s, OCH₃), 4.08 (1H, m, CHNH (AHPA)), 4.18 (1H, dd, J=1.8 Hz, 6.5 Hz, CHOH (AHPA)), 4.27 (1H, m, CHNH (Leu)), 4.50 (1H, t, CHC═C), 5.92 (1H, d, J=5.8 Hz, C═CH), 6.31 (1H, s, ArH {A-ring}), 6.66-6.91 (3H, m, 3×ArH {C-ring}), 7.16-7.37 (5H, m, 5×ArH (AHPA).

¹³C NMR (CDCl₃) δ_{c} ppm: 21.9 (CH₃ (Leu)), 22.3 (CH₂), 23.2 (CH₃ (Leu)), 24.8 (CH(CH₃)₂ (Leu)), 28.3 (3×C(CH₃)₃ (Boc)), 41.1 (CH₂ (AHPA)), 41.4 (CH₂ (Leu), 41.5 (CH₂), 48.1 (CHNH (Leu)), 51.5 (CHC═C), 55.6 (CHNH (AHPA)), 56.1 (2×OCH₃), 60.9 (OCH₃), 61.6 (OCH₃), 73.8 (CHOH (AHPA)), 80.4 (C(CH₃)₃ (Boc)), 109.1 (2×ArCH), 114.5 (ArCH), 120.1 (ArCH), 126.6 (ArCH), 127.3 (ArC), 128.1 (ArCH), 128.7 (2×ArCH), 129.3 (2×ArCH), 134.7 (ArC), 135.1 (ArC), 138.1 (ArC), 140.6 (ArC), 141.6 (ArC), 145.3 (ArC), 146.6 (ArC), 150.9 (ArC), 151.3 (ArC), 157.2 (NHC═O (Boc)), 171.1 (NHC═O (AHPA)), 173.1 (NHC═O (Leu)).

νₘₐₓ/cm⁻¹ 3309.8, 2928.1, 1647.9, 1508.3, 1367.0, 1168.9

### Synthesis of 4.37—Removal of Fmoc from Aniline (B-Ring Amide Bestatin)

To a stirred solution of 4.35 (70 mg, 0.07 mmol) in THF (1 mL) was added 1 M TBAF (0.07 mL, 0.07 mmol) at 0° C. After 40 min the reaction volume was loaded directly onto a flash column (stationary phase; silica gel 230-400 mesh, mobile phase; 1:1, hexane/ethyl acetate). All homogenous fractions were collected and the solvent was evaporated to afford the product as a white solid (50 mg, 100%).

¹H NMR (CDCl₃) δ_{H} ppm: 0.93 (6H, dd, J=4.7 Hz, 19.2 Hz, 2×CH₃ (Leu)), 1.36 (9H, s, C(CH₃)₃ (Boc)), 1.61 (3H, m, CH₂ (Leu), CH (Leu)), 1.84 (1H, m, CH₂), 2.14 (1H, m, CH₂), 2.39 (1H, m, CH₂), 2.93 (3H, m, 2×CH₂ (AHPA bzl), CH₂), 3.56 (3H, s, OCH₃), 3.85 (3H, s, OCH₃), 3.88 (3H, s, OCH₃), 3.90 (3H, s, OCH₃), 4.10 (1H, m, CHNH (AHPA)), 4.15 (CHOH (AHPA)), 4.22 (H, m, CHNH (Leu)), 4.65 (1H, t, CHC═C), 5.78 (1H, d, J=5.4 Hz, C═CH), 6.25 (1H, s, ArH {A-ring}), 6.40 (1H, d, J=6.7 Hz, ArH {C-ring}), 6.62 (2H, m, 2×ArH {C-ring}), 7.20 (5H, m, 5×ArH (AHPA)).

¹³C NMR (CDCl₃) δ_{c} ppm: 22.1 (CH₃ (Leu)), 22.2 (CH₂), 23.0 (CH₃ (Leu)), 24.7 (CH(CH₃)₂ (Leu)), 28.3 (3×C(CH₃)₃ (Boc)), 37.8 (CH₂ (AHPA)), 41.0 (CH₂), 41.4 (CH₂ (Leu)), 47.9 (CHNH (AHPA)), 48.3 (CHNH (Leu)), 50.6 (CHC═C), 55.5 (OCH₃), 55.9 (OCH₃), 60.9 (OCH₃), 61.6 (OCH₃), 72.9 (CHOH (AHPA)), 79.8 (C(CH₃)₃ (Boc)), 109.2 (ArCH), 109.8 (ArCH), 115.0 (ArCH), 119.3 (ArCH), 126.5 (ArCH), 127.1 (ArC), 128.2 (ArCH), 128.5 (2×ArCH), 129.4 (2×ArCH), 134.5 (ArC), 135.4 (ArC), 135.5 (ArC), 138.0 (ArC), 140.7 (ArC), 141.3 (ArC), 147.1 (ArC), 150.6 (ArC), 151.2 (ArC), 156.3 (NHC═O (Boc)), 171.8 (NHC═O (AHPA)), 173.0 (NHC═O (Leu)).

νₘₐₓ/cm⁻¹ 2930.0, 1642.0, 1523.6, 1230.8, 1109.6, 860.9

### Synthesis of 4.38 and 4.39—Removal of the N-Boc Group

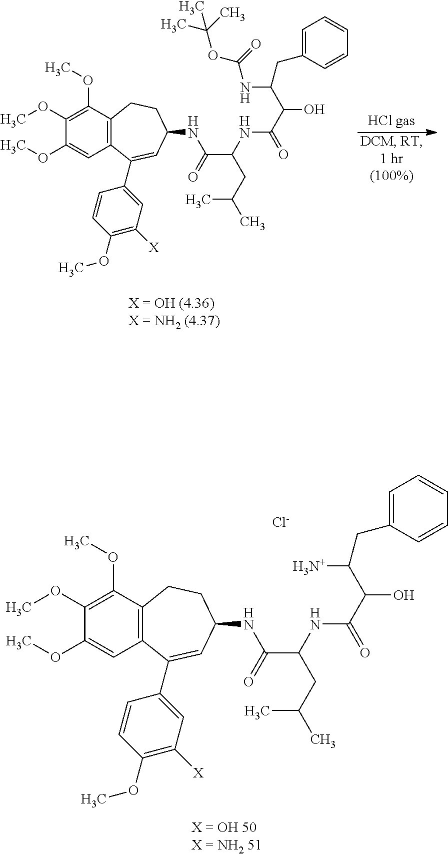

### Synthesis of 4.38—Removal of the N-Boc Group

A stirred solution of 4.36 (25 mg, 0.03 mmol) in DCM was acidified with HCl gas. This was left stirring at room temperature for one hour until all of the starting material had been deprotected, as shown by TLC. The solvent was removed in vacuo to afford the product 50 as an off-white solid (20 mg, 100%).

¹H NMR (CD₃OD) δ_{H} ppm: 1.00 (6H, t, 2×CH₃ (Leu)), 1.32 (1H, m, CH (Leu)), 1.64 (3H, m, CH₂ (Leu), CH₂), 2.08 (1H, m, CH₂), 2.34 (1H, m, CH₂), 2.93 (1H, dd, J=6.5 Hz, 13.5 Hz, CH₂ (AHPA bzl)), 3.12 (2H, dm, CH₂, CH₂ (AHPA bzl)), 3.68 (3H, s, OCH₃), 3.79 (1H, t, CHNH (AHPA)), 3.89 (6H, s, OCH₃), 3.91 (3H, s, OCH₃), 4.17 (1H, d, J=3.6 Hz, CHOH (AHPA)), 4.18 (1H, m, CHNH (Leu)), 4.39 (1H, dd, J=6.3 Hz, 8.7 Hz, CHC═C), 6.10 (1H, d, J=5.7 Hz, C═CH), 6.41 (1H, s, ArH {A-ring}), 6.69-6.75 (2H, td, J=2.0 Hz, 8.4 Hz, 17.8 Hz, 2×ArH {C-ring}), 6.88 (1H, d, J=7.2 Hz, ArH {C-ring}), 7.29-7.41 (5H, m, 5×ArH (AHPA).

¹³C NMR (CD₃OD) δ_{c} ppm: 20.9 (CH₃ (Leu)), 21.3 (CH₂), 21.7 (CH₃ (Leu)), 24.8 (CH(CH₃)₂ (Leu)), 34.8 (CH₂ (AHPA)), 40.1 (CH₂), 40.2 (CH₂ (Leu), 48.1 (CHNH (Leu)), 52.0 (CHC═C), 54.6 (CHNH (AHPA)), 54.7 (2×OCH₃), 60.9 (OCH₃), 61.6 (OCH₃), 68.0 (CHOH (AHPA)), 109.1 (2×ArCH), 114.5 (ArCH), 120.1 (ArCH), 126.6 (ArCH), 127.3 (ArC), 128.1 (ArCH), 128.7 (2×ArCH), 129.3 (2×ArCH), 134.7 (ArC), 135.1 (ArC), 138.1 (ArC), 140.6 (ArC), 141.6 (ArC), 145.3 (ArC), 146.6 (ArC), 150.9 (ArC), 151.3 (ArC), 171.1 (NHC═O (AHPA)), 173.1 (NHC═O (Leu)).

νₘₐₓ/cm⁻¹ 3235.2, 2944.4, 2843.3, 1629.7, 1355.8, 1246.3, 1111.4, 1023.0, 799.6.

### Synthesis of 4.39—Removal of the N-Boc Group

A stirred solution of 4.37 (25 mg, 0.03 mmol) in DCM was acidified with HCl gas. This was left stirring at room temperature for one hour until all of the starting material had been deprotected, as shown by TLC. The solvent was removed in vacuo to afford the product 51 as a beige-coloured solid (20 mg, 100%).

¹H NMR (CD₃OD) δ_{H} ppm: 0.97 (6H, dd, J=4.7 Hz, 19.2 Hz, 2×CH₃ (Leu)), 1.63 (2H, m, CH₂ (Leu)), 1.66 (1H, m, CH (Leu)), 2.04 (1H, m, CH₂), 2.33 (1H, m, CH₂), 2.35 (1H, m, CH₂), 2.66 (H, m, CH₂ (AHPA bzl), 2.92 (H, m, CH₂ (AHPA bzl), 3.06 (1H, m, CH₂), 3.35 (1H, t, CHNH (AHPA)), 3.66 (3H, s, OCH₃), 3.87 (3H, s, OCH₃), 3.89 (3H, s, OCH₃), 3.90 (3H, s, OCH₃), 3.98 (1H, d, J=4.0 Hz, CHOH (AHPA)), 4.19 (1H, m, CHNH (Leu)), 5.98 (1H, d, J=8.1 Hz, CHC═C), 6.05 (1H, d, J=5.7 Hz, C═CH), 6.40 (1H, s, ArH {A-ring}), 6.62 (1H, dd, J=2.3 Hz, 8.3 Hz, ArH {C-ring}), 6.68 (H, d, J=2.0 Hz, ArH {C-ring}), 6.76 (H, d, J=9.0 Hz, ArH {C-ring}), 7.19-7.33 (5H, m, 5×ArH (AHPA)).

¹³C NMR (CD₃OD) δ_{c} ppm: 21.1 (CH₃ (Leu)), 21.5 (CH₂), 22.1 (CH₃ (Leu)), 24.4 (CH(CH₃)₂ (Leu)), 39.4 (CH₂ (AHPA)), 41.0 (CH₂), 41.4 (CH₂ (Leu)) 47.8 (CHNH (Leu)), 51.6 (CHC═C), 54.3 (OCH₃), 55.1 (OCH₃), 55.1 (CHNH (AHPA)), 60.0 (OCH₃), 60.0 (OCH₃), 72.8 (CHOH (AHPA)), 109.1 (ArCH), 109.8 (ArCH), 115.0 (ArCH), 118.3 (ArCH), 126.2 (ArCH), 127.2 (ArCH), 127.4 (ArC), 128.3 (2×ArCH), 129.0 (2×ArCH), 133.9 (ArC), 135.7 (ArC), 135.8 (ArC), 138.7 (ArC), 141.0 (ArC), 141.3 (ArC), 147.6 (ArC), 150.7 (ArC), 151.2 (ArC), 172.3 (NHC═O (AHPA)), 174.1 (NHC═O (Leu)).

νₘₐₓ/cm⁻¹ 2932.9, 1647.3, 1512.6, 1237.4, 1112.5, 839.7

### Synthesis of 52

### Synthesis of tert-butyl 2-((benzyloxy)carbonyl)-1-(methoxycarbonyl)ethyl carbamate 52.2

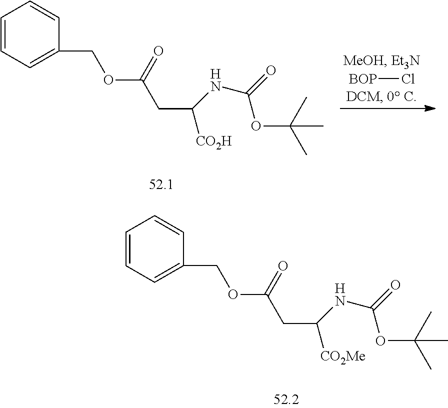

To a stirred solution of the acid (52.1) (2.50 g, 7.73 mmol) in anhydrous DCM (30 mL) was added methanol (0.63 mL, 15.46 mmol), Et₃N (1.97 mL, 15.46 mmol) and bis-(2-oxo-3-oxazolidinyl)-phosphorylchloride (BOP—Cl) (1.97 g, 7.73 mmol) at 0° C. under dry reaction conditions. After 16 h the reaction was quenched by the addition of water (1×50 mL). The product was extracted with diethyl ether (3×50 mL). The combined organic extracts were washed with 5% aq. NaHCO₃ (1×50 mL) and water (1×50 mL), respectively. The organic fraction was then dried over MgSO₄, filtered and concentrated in vacuo. The product was purified by flash column chromatography (stationary phase; silica gel 230-400 mesh, mobile phase; 2:1, hexane/ethyl acetate). All homogenous fractions were collected and the solvent was evaporated to afford 52.2 as white crystalline solid (1.90 g, 73%).

¹H NMR (CDCl₃, 400 MHz) δ_{H} ppm: 1.46 (9H, s, C(CH₃)₃), 2.89 (1H, dd, J=4.5 Hz, 17.0 Hz, CH₂CH), 3.06 (1H, dd, J=4.5 Hz, 17.0 Hz, CH₂CH), 3.71 (3H, s, OMe), 4.61 (1H, m, CHNH), 5.14 (2H, q, J=4.0 Hz, 12.0 Hz, BzCH₂), 5.52 (1H, d, J=8.5 Hz, NH), 7.35 (5H, m, 5×ArH)

¹³C NMR (CDCl₃, 400 MHz) δ_{c} ppm: 27.83 (C(CH₃)₃), 36.43 (CH₂CH), 49.49 (CHNH), 52.22 (OMe), 66.34 (BzCH₂), 79.70 (C(CH₃)₃), 127.87 (2×ArCH), 127.97 (1×ArCH), 128.15 (2×ArCH), 134.93 (ArC), 154.90 (C═O), 170.33 (C═O), 171.05 (C═O)

νₘₐₓ (KBr)/cm⁻¹ 3370.6, 2978.0, 1737.9, 1165.3

Melting point: 55-57° C.

HRMS: calculated 360.1423, found 360.1426, molecular formula (C₁₇H₂₃NO₆Na).

General procedure for N—BOC Deprotection—Amino Acid Coupling—Formation of TFA Salts 52.3a-c

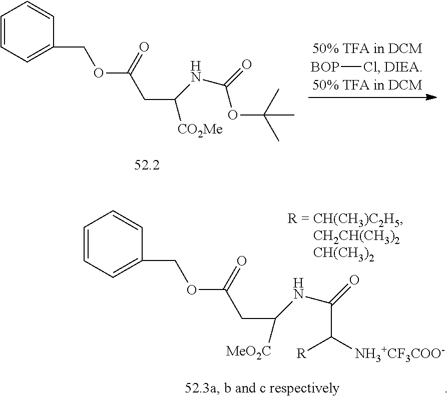

To a dry three-necked round bottom flask containing N—BOC protected amine 52.2 (1 mmol) dissolved in dry DCM (5 mL) was added TFA (5 mL) drop-wise under an atmosphere of nitrogen at 0° C. After 20 min the solvent was removed from the flask under vacuum. The residue was dissolved in toluene (50 mL) and the flask was heated under vacuum to azeotropically remove any traces of TFA. The resulting TFA salts were dried in vacuo for several hours and were used without further purification or characterisation.

### Synthesis of the dipeptide Asp-Ile

To a stirred solution of the TFA salt of 52.2 (610 mg, 1.74 mmol) in anhydrous DCM (12 mL) was added N—BOC isoleucine (402 mg, 1.74 mmol) followed by N,N-diisopropylethylamine (0.61 mL, 3.48 mmol) and BOP—Cl (443 mg, 1.74 mmol) under dry reaction conditions at 0° C. The reaction was left stirring for 20-24 h. It was quenched by the addition of water (1×10 mL) and the product was extracted with diethyl ether (3×25 mL). The combined organic extracts were washed with 5% aq. NaHCO₃ (1×30 mL), water (1×30 mL) and were dried over MgSO₄, filtered and concentrated to afford an off-white solid. The product was purified by flash column chromatography (stationary phase; silica gel 230-400 mesh, mobile phase; 2:1, hexane/ethyl acetate). All homogenous fractions were collected and the solvent was evaporated to afford the product 52.3a as a white solid (480 mg, 61%).

¹H NMR (CDCl₃, 400 MHz) δ_{H} ppm: 0.91 (6H, m, (2×CH₃) (Ile)), 1.45 (11H, s, (C(CH₃)₃ & CH₂) (Ile)), 1.87 (1H, m, CH (Ile)), 2.88 (1H, dd, J=4.5 Hz, 17.0 Hz, CH₂CH (Asp)), 3.10 (1H, dd, J=4.5 Hz, 17.0 Hz CH₂CH (Asp)), 3.70 (3H, s, OMe), 4.01 (1H, m, CHNH (Ile)), 4.88 (1H, m, CHNH (Asp)), 5.11 (1H, d, J=8.0 Hz, NH (Ile)), 5.11 (2H, s, BzCH₂), 6.90 (1H, d, J=7.5 Hz, NH (Asp)), 7.35 (5H, m, 5×ArH)

¹³C NMR (CDCl₃, 400 MHz) δ_{c} ppm: 11.10 (CH₃ (Ile)), 14.96 (CH₃ (Ile)), 24.15 (CH₂ (Ile)), 27.85 (C(CH₃)₃), 35.76 (CH₂ (Asp)), 37.22 (CH (Ile)), 47.93 (CHNH (Asp)), 52.28 (OMe), 58.61 (CHNH (Ile)), 66.47 (BzCH₂), 79.42 (C(CH₃)₃), 128.42 (2×ArCH), 128.51 (ArCH), 128.64 (2×ArCH), 134.80 (ArC), 155.03 (C═O), 170.32 (C═O), 170.39 (C═O), 172.86 (C═O)

νₘₐₓ (KBr)/cm⁻¹ 3318.5, 2965.0, 1740.6, 1655.3, 1522.1, 1169.0

Melting point: 41-44° C.

HMRS: calculated 473.2264, found 473.2285, molecular formula (C₂₃H₃₄N₂O₇Na).

### Synthesis of the dipeptide Asp-Leu

To a stirred solution of the TFA salt of 52.2 (660 mg, 1.88 mmol) in anhydrous DCM (12 mL) was added N—BOC leucine (469 mg, 1.88 mmol) followed by N,N-diisopropylethylamine (0.70 mL, 3.76 mmol) and BOP—Cl (480 mg, 1.88 mmol) under dry reaction conditions at 0° C. The reaction was left stirring for 24 h. It was quenched by the addition of water (1×10 mL) and the product was extracted with diethyl ether (3×25 mL). The combined organic extracts were washed with 5% aq. NaHCO₃ (1×30 mL), water (1×30 mL) and were dried over MgSO₄, filtered and concentrated to afford an oil. The product was purified by flash column chromatography (stationary phase; silica gel 230-400 mesh, mobile phase; 2:1, hexane/ethyl acetate). All homogenous fractions were collected and the solvent was evaporated to afford the product 52.2b as a colourless oil (280 mg, 33%).

¹H NMR (CDCl₃, 400 MHz) δ_{H} ppm: 0.91 (6H, m, (2×CH₃) (Leu)), 1.47 (10H, s, C(CH₃)₃ & CH(CH₃)₂ (Leu)), 1.63 (2H, m, CH₂ (Leu)), 2.89 (1H, dd, J=5.0 Hz, 17.0 Hz, CH₂CH (Asp)), 3.04 (1H, dd, J=4.5 Hz, 17.0 Hz, CH₂CH (Asp)), 3.68 (3H, s, OMe), 4.12 (1H, m, CHNH (Leu)), 4.85 (1H, m, CHNH (Asp)), 5.02 (1H, d, J=7.5 Hz, NH (Leu)), 5.11 (2H, s, BzCH₂), 7.07 (1H, d, J=8.0 Hz, NH (Asp)), 7.35 (5H, m, 5×ArH)

¹³C NMR (CDCl₃, 400 MHz) δ_{c} ppm: 21.46 (CH₃ (Leu)), 22.47 (CH₃ (Leu)), 24.19 (CH(CH₃)₂ (Leu)), 27.82 (C(CH₃)₃), 35.81 (CH₂ (Asp)), 40.91 (CH₂ (Leu)), 48.02 (CHNH (Asp)), 52.24 (OMe), 52.59 (CHNH (Leu)), 66.36 (BzCH₂), 79.42 (C(CH₃)₃), 127.94 (2×ArCH), 127.97 (ArCH), 128.14 (2×ArCH), 134.90 (ArC), 155.03 (C═O), 170.18 (C═O), 170.47 (C═O), 172.09 (C═O)

νₘₐₓ (DCM)/cm⁻¹ 3314.9, 2596.9, 1740.7, 1664.3, 1518.0, 1170.1

HRMS: calculated 473.2264, found 473.2259, molecular formula (C₂₃H₃₄N₂O₇Na).

### Synthesis of the Dipeptide Asp-Val

To a stirred solution of the TFA salt of 52.2 (650 mg, 1.85 mmol) in anhydrous DCM (12 mL) was added N—BOC valine (402 mg, 1.85 mmol) followed by N,N-diisopropylethylamine (0.65 mL, 3.70 mmol) and BOP—Cl (470 mg, 1.85 mmol) under dry reaction conditions at 0° C. The reaction was left stirring for 24 h. It was quenched by the addition of water (1×10 mL) and the product was extracted with diethyl ether (3×25 mL). The combined organic extracts were washed with 5% aq. NaHCO₃ (1×30 mL), water (1×30 mL) and were dried over MgSO₄, filtered and concentrated to afford an off-white solid. The product was purified by flash column chromatography (stationary phase; silica gel 230-400 mesh, mobile phase; 2:1, hexane/ethyl acetate). All homogenous fractions were collected and the solvent was evaporated to afford the product 52.3c as a white solid, (200 mg, 25%).

¹H NMR (CDCl₃, 400 MHz) δ_{H} ppm: 0.88 (3H, d, J=7.0 Hz, CH₃ (Val)), 0.96 (3H, d, J=6.5 Hz, CH₃ (Val)), 1.44 (9H, s C(CH₃)₃), 2.13 (1H, m, CH(CH₃)₂ (Val)), 2.89 (1H, dd, J=4.5 Hz, 17.0 Hz, CH₂CH (Asp)), 3.09 (1H, dd, J=4.5 Hz, 17.0 Hz, CH₂), 3.70 (3H, s, OMe), 3.99 (1H, m, CHNH (Val)), 4.86 (1H, m, CHNH (Asp)), 5.10 (1H, d, J=7.5 Hz, NH (Val)), 5.12 (2H, s, BzCH₂ (Asp)), 6.90 (1H, m, NH (Asp)), 7.35 (5H, m, 5×ArH)

¹³C NMR (CDCl₃, 400 MHz) δ_{c} ppm: 17.00 (CH₃ (Val)), 18.63 (CH₃ (Val)), 27.83 (C(CH₃)₃), 30.73 (CH(CH₃)₂) (Val)), 35.54 (CH₂CH (Asp)), 47.96 (CHNH (Asp)), 52.32 (OMe), 59.16 (CHNH (Val)), 66.47 (BzCH₂), 79.39 (C(CH₃)₃), 127.96 (2×ArCH), 128.03 (1×ArCH), 128.17 (2×ArCH), 134.93 (ArC), 155.27 (C═O), 170.31 (C═O), 170.40 (C═O), 171.93 (C═O)

νₘₐₓ (KBr)/cm⁻¹ 3322.7, 2965.0, 1740.9, 1660.4, 1519.7, 1170.7

Melting point: 65-67° C.

HRMS: calculated 459.2107, found 459.2112, molecular formula (C₂₂H₃₂N₂O₇Na).

### Synthesis of the tripeptide Asp-Leu-AHPA 52.7

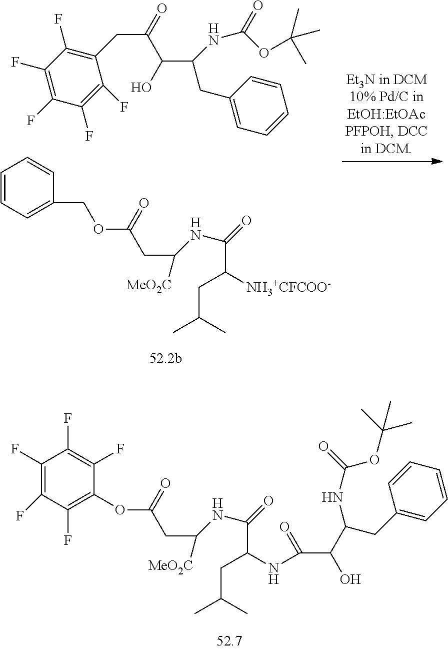

To a dry three-necked round bottom flask containing PFP ester of N—BOC-AHPA (50 mg, 0.11 mmol) dissolved in anhydrous DCM (12 mL) was added 52.2b (80 mg, 0.18 mmol) followed by Et₃N (0.05 mL, 0.36 mmol) under dry reaction conditions. The reaction was allowed to stir for twelve h at room temperature. The reaction was quenched by the addition of water (1×30 mL) and the product was extracted with DCM (3×30 mL). The combined organic fractions were washed with 5% aq. NaHCO₃ (2×30 mL) and water (2×30 mL). The organic layer was dried over MgSO₄, filtered and concentrated to afford an off-white solid. The product was purified by flash column chromatography (stationary phase; silica gel 230-400 mesh, mobile phase; 4:1, hexane/ethyl acetate). All homogenous fractions were collected and the solvent was evaporated to afford the product 52.7 as a white solid, quantitatively.

¹H NMR (CDCl₃, 400 MHz) δ_{H} ppm: 0.93 (6H, m, (2×CH₃) (Leu)), 1.39 (9H, s, (C(CH₃)₃), 1.63 (3H, m, CH & CH₂ (Leu)), 3.00 (4H, m CH₂CH (Asp), CH₂ (AHPA)), 3.69 (3H, s, OMe), 4.03 (1H, m, CHNH (AHPA), 4.14 (1H, m, CHOH), 4.51 (1H, m, CHNH (Leu)), 4.84 (1H, m, CHNH (Asp)), 5.10 (2H, s, BzCH₂ (Asp)), 5.15 (1H, m, NH (AHPA)), 7.27 (12H, m, NH (Leu), NH (Asp) & 10×ArH))

¹³C NMR (CDCl₃, 400 MHz) δ_{c} ppm: 21.24 (CH₃ (Leu)), 22.63 (CH₃ (Leu)), 24.12 (CH (Leu)), 27.68 (C(CH₃)₃), 35.72 (CH₂ (Asp)), 35.84 (CH₂, (AHPA)), 40.31 (CH₂ (Leu)), 40.15 (CHNH (Asp)), 50.84 (CHNH (Leu)), 52.33 (OMe), 55.21 (CHNH (AHPA)), 66.43 (BzCH₂ (Asp)), 73.54 (CHOH), 79.93 (C(CH₃)₃), 126.14 (ArCH), 127.93 (ArCH), 127.97 (2×ArCH), 128.02 (ArCH), 128.08 (2×ArCH), 128.17 (2×ArCH), 128.86 (ArCH) 134.81 (ArC), 137.62 (ArC), 156.96 (C═O), 170.08 (C═O), 170.25 (C═O), 170.37 (C═O), 171.29 (C═O), 172.53 (C═O)

νₘₐₓ (KBr)/cm⁻¹ 3199.1, 3065.7, 2958.8, 1724.1, 1684.3, 1262.2

Melting point: 125-130° C.

HMRS: calculated 650.3054, found 650.3046, molecular formula (C₃₃H₄₅N₃O₉Na).

### Hydrogenolysis of the Benzyl Ester of Tripeptide N—BOC—O-Benzyl-Asp-Leu-AHPA

To a stirred solution of the tripeptide Asp-Leu-AHPA (60 mg, 0.10 mmol) in a 1:1 mixture of ethanol and ethyl acetate (4 mL) was added 10% Pd/C (catalytic amount). The mixture was stirred at room temperature under an atmosphere of hydrogen (balloon). The reaction was monitored by TLC. After 3 h the Pd/C was removed from the reaction mixture by filtration using DCM (200 mL). The solvent was removed from the flask under vacuum and the resulting acid was obtained as a white solid (50 mg, 97%)

¹H NMR (CDCl₃, 400 MHz) δ_{H} ppm: 0.92 (6H, m, (2×CH₃) (Leu)), 1.36 (9H, s, (C(CH₃)₃), 1.65 (3H, m, CH & CH₂ (Leu)), 2.92 (4H, m CH₂CH (Asp), CH₂ (AHPA)), 3.76 (3H, s, OMe), 4.14 (2H, m, CHNH & CHOH (AHPA)), 4.89 (2H, m, CHNH (Leu) & CHNH (Asp)), 5.22 (1H, m, NH (AHPA), 7.22 (5H, m, 5×ArH), 7.76 (2H, m, NH (Leu), NH (Asp))

¹³C NMR (CDCl₃, 400 MHz) δ_{c} ppm: 21.47 (CH₃ (Leu)), 22.36 (CH₃ (Leu)), 24.16 (CH (Leu)), 27.78 (C(CH₃)₃), 35.66 (CH₂ (Asp)), 36.37 (CH₂, (AHPA)), 40.80 (CH₂ (Leu)), 47.68 (CHNH (Asp)), 50.71 (CHNH (Leu)), 52.32 (OMe), 54.81 (CHNH (AHPA)), 60.00 (CHOH), 79.82 (C(CH₃)₃), 126.10 (ArCH), 128.03 (2×ArCH), 128.88 (2×ArCH), 137.46 (ArC), 156.96 (C═O), 170.40 (3×C═O)

νₘₐₓ (KBr)/cm⁻¹ 3325.1, 2931.6, 1651.0, 1519.8, 1172.6

Melting point: 75-80° C.

HRMS: calculated 560.2584, found 560.2592, molecular formula (C₂₆H₃₉N₃O₉Na).

### Synthesis of the PFP Ester of Asp-Leu-AHPA 52.7

To a stirred solution of the acid (50 mg, 0.09 mmol) in anhydrous DCM (2 mL) was added pentafluorophenol (17 mg, 0.09 mmol) followed by DCC (19 mg, 0.09 mmol) at 0° C. under dry reaction conditions. The temperature was allowed to increase to ambient after 10 min. The reaction was left stirring for an additional 2 h. The reaction was filtered using DCM (50 mL) and the filtrate was washed with 5% aq. NaHCO₃ (2×30 mL) and water (1×30 mL). The organic layer was dried over MgSO₄, filtered and concentrated to afford an off-white solid. The product was purified by flash column chromatography (stationary phase; silica gel 230-400 mesh, mobile phase; 4:1, hexane/ethyl acetate). All homogenous fractions were collected and the solvent was evaporated to afford the product 52.7 as a white solid (50 mg, 71%). The product was used in the ensuing coupling step without any further purification or characterisation.

### Synthesis of 52.12

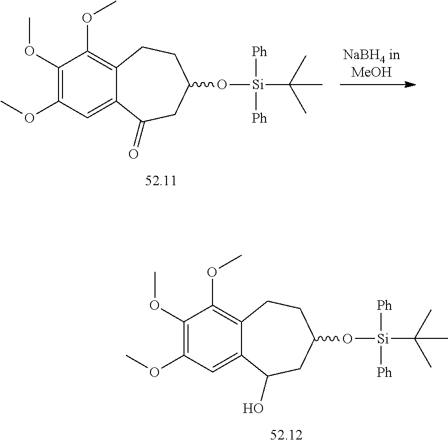

To a stirred solution of 52.11 (1.20 g, 2.38 mmol) in methanol (10 mL) and THF (5 mL) was added NaBH₄ (210 mg, 2.38 mmol) at 0° C. After 1 h the reaction was quenched by the addition of sat. aq. NaCl (1×40 mL) and the product was extracted with diethyl ether (3×50 mL). The combined etheral fractions were dried over MgSO₄, filtered and concentrated to afford a yellow oil. The product was purified by flash column chromatography (stationary phase; silica gel 230-400 mesh, mobile phase; 4:1, hexane/ethyl acetate). All homogenous fractions were collected and the solvent was evaporated to afford the product 52.12 as a white solid (1.10 g, 92%)

¹H NMR (CDCl₃, 400 MHz) δ_{H} ppm: 1.14 (9H, s, major diastereomer, C(CH₃)₃), 1.20 (9H, s, minor diastereomer, C(CH₃)₃), 1.41-1.90 (4H, major & minor diastereomers, CH₂), 3.02-3.26 (2H, m, major & minor diastereomers, CH₂), 3.80 (3H, s, major diastereomer, OMe), 3.81 (3H, s, minor diastereomer, OMe), 3.83 (3H, s, major diastereomer, OMe), 3.84 (3H, s, minor diastereomer, OMe), 3.88 (3H, s, major diastereomer, OMe), 3.89 (3H, s, minor diastereomer, OMe), 4.21 (1H, m, minor diastereomer, CH), 4.32 (1H, m, major diastereomer, CH), 4.71 (1H, m, diastereomer, CH), 5.46 (1H, m, diastereomer, CH), 6.84 (1H, s minor diastereomer, ArH) 6.98 (1H, s, major diastereomer, ArH), 7.44 (6H, m, major & minor diastereomers, ArH), 7.78 (4H, m, major & minor diastereomers, ArH)

¹³C NMR (CDCl₃, 400 MHz) δ_{c} ppm: 17.38, 18.06 (C(CH₃)₃), 18.73, 19.01 (CH₂), 26.57, 26.72 (C(CH₃)₃), 34.58, 35.28 (CH₂), 44.35 (CH₂), 55.45, 55.48 (OMe), 60.04, 60.41 (OMe), 60.43, 60.92 (OMe), 69.16 (CHOP), 73.42 (CHOH), 103.37 (ArCH), 125.32 (ArC), 127.27, 127.30 (4×ArCH), 129.33, 129.36 (ArCH), 129.43, 129.45 (ArCH), 133.31, 134.03 (ArC), 135.41, 135.45 (4×ArCH), 139.35, 140.90 (ArC), 140.47, 140.90 (ArC), 150.41, 150.50 (ArC), 150.57, 150.60 (ArC)

νₘₐₓ (DCM)/cm⁻¹ 3468.0, 2932.6, 1600.1, 1114.3, 703.0

Melting point: 55-57° C.

HRMS: calculated 529.2386, found 529.2387, molecular formula (C₃₀H₃₈O₅NaSi).

### Synthesis of 52.13

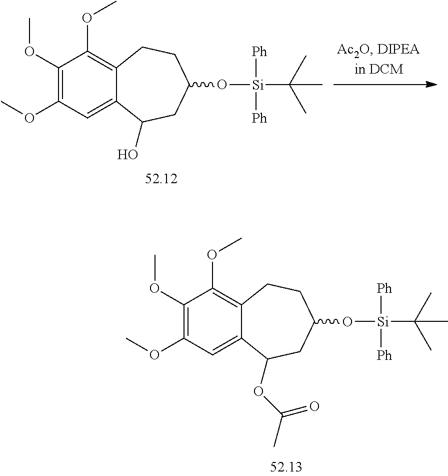

To a stirred solution of 52.12 (1.34 g, 2.65 mmol) in anhydrous DCM (30 mL) was added acetic anhydride (0.50 mL, 5.30 mmol), DMAP (320 mg, 2.65 mmol) and N,N-diisopropylethylamine (0.92 mL, 5.30 mmol) under dry reaction conditions at 0° C. After 3 h the reaction was quenched by the addition of 2M aq. HCl (1×50 mL) and the product was extracted with diethyl ether (3×60 mL). The combined etheral fractions were dried over MgSO₄, filtered and concentrated to afford a yellow oil. The product was purified by flash column chromatography (stationary phase; silica gel 230-400 mesh, mobile phase; 4:1, hexane/ethyl acetate). All homogenous fractions were collected and the solvent was evaporated to afford the product 52.13 as a colourless oil, (1.42 g, 100%)

¹H NMR (CDCl₃, 400 MHz) δ_{H} ppm: 1.10 (9H, s, major diastereomer, C(CH₃)₃), 1.15 (9H, s, minor diastereomer, C(CH₃)₃), 1.39 (2H, m, major & minor diastereomers, CH₂), 1.90 (2H, m, major & minor diastereomers, CH₂), 2.20 (3H, s, major & minor diastereomers, COCH₃), 2.78 (2H, m, minor diastereomer, CH₂), 3.19 (2H, m, major diastereomer, CH₂), 3.82 (3H, s, major diastereomer, OMe), 3.86 (3H, s, minor diastereomer, OMe), 3.88 (6H, s, minor diastereomer, 2×OMe), 3.90 (6H, s, major diasteroemer, OMe), 4.15, (2H, m, major & minor diastereomers, 2×CH), 6.64 (1H, s, minor diastereomer, ArH), 6.70 (1H, s, major diastereomer, ArH), 7.44 (6H, m, major & minor diastereomers, 6×ArH), 7.74 (4H, m, major & minor diastereomers, 4×ArH)

¹³C NMR (CDCl₃, 400 MHz) δ_{c} ppm: 18.17, 18.73 (C(CH₃)₃), 18.79, 18.82 (CH₂), 20.65, 20.80 (COCH₃), 26.53, 26.59 (C(CH₃)₃), 35.65, 35.86 (CH₂), 40.95, 43.30 (CH₂), 55.57, 55.58 (OMe), 59.94, 60.39 (OMe), 60.88, 60.92 (OMe), 69.23, 69.65 (CHOP), 72.51 (CH), 102.70 (ArCH), 124.67 (ArC), 127.16, 127.19 (4×ArCH), 129.23 (2×ArCH), 133.75, 133.80 (ArC), 135.35, 135.41 (2×ArCH), 135.45, 135.50 (2×ArCH), 135.62 (ArC), 140.76, 141.00 (ArC), 150.48, 150.57 (ArC), 150.85, 150.94 (ArC), 169.04 (C═O)

νₘₐₓ (DCM)/cm^{˜}2932.9, 1738.0, 1601.4, 1236.4, 1112.7, 703.1

### Synthesis of 6,7,8,9-tetrahydro-7-hydroxy-1,2,3-trimethoxy-5H-benzo[7]annulen-5-yl acetate 52.14

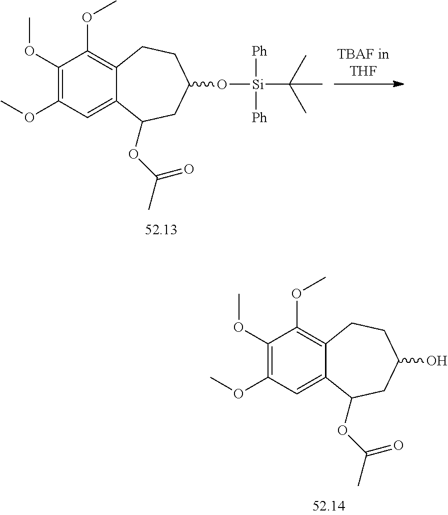

To a stirred solution of 52.13 (400 mg, 0.73 mmol) in THF (2 mL) was added 1M TBAF in THF (0.73 mL, 0.73 mmol) at 0° C. After 6 h the reaction mixture was applied directly to a flash column. The product was purified by flash column chromatography (stationary phase; silica gel 230-400 mesh, mobile phase; 1:1, hexane/ethyl acetate). All homogenous fractions were collected and the solvent was evaporated to afford the product 52.14 as a colourless oil (230 mg, 100%).

¹H NMR (CDCl₃, 400 MHz) δ_{H} ppm: 1.77 (2H, m, major & minor diasteromers, CH₂), 2.09 (3H, s, minor diastereomer, COCH₃), 2.19 (3H, s, major diastereomer, COCH₃), 2.24-3.28 (4H, m, major & minor diastereomers, 2×CH₂), 3.78 (3H, s, minor diastereomer, OMe), 3.79 (3H, s, major diastereomer, OMe), 3.83 (6H, s, minor diastereomer, 2×OMe), 3.85 (6H, s, major diastereomer, 2×OMe), 4.07 (2H, m, major & minor diastereomers, 2×CH), 6.66 (1H, s, minor diasteroemer, ArH), 6.69 (1H, s, major diastereomer, ArH)

¹³C NMR (CDCl₃, 400 MHz) δ_{c} ppm: 18.42, 18.82 (CH₂), 20.80, 20.88 (COCH₃), 35.37, 35.45 (CH₂), 40.44, 42.39 (CH₂), 55.54 (OMe), 59.96, 60.34 (OMe), 60.37, 60.91 (OMe), 68.23 (CH), 70.10, 70.90 (CH), 103.36 (ArCH), 124.76, 126.77 (ArC), 135.23 (ArC), 140.84, 141.32 (ArC), 150.46, 150.60 (ArC), 150.81, 150.89 (ArC), 169.29, 169.65 (C═O)

νₘₐₓ (DCM)/cm⁻¹ 3440.4, 2934.5, 1736.0, 1601.5, 1238.5, 1120.8

### Synthesis of 7-azido-6,7,8,9-tetrahydro-1,2,3-trimethoxy-5H-benzo[7]annulen-5-yl acetate 52.16

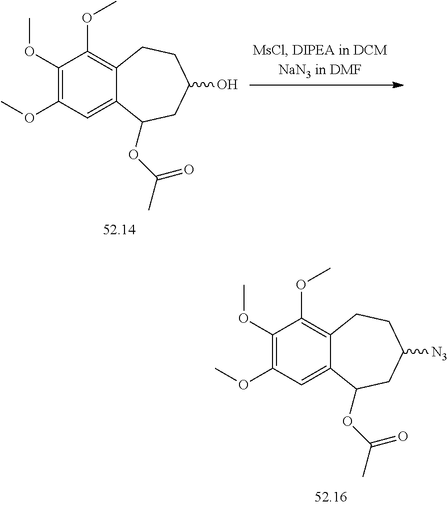 [SMILES: COc1cc2c(c(OC)c1OC)CCC(O)CC2OC(C)=O ; COc1cc2c(c(OC)c1OC)CCC(N=[N+]=[N-])CC2OC(C)=O]

To a stirred solution of 52.14 (360 mg, 1.17 mmol) in anhydrous DCM (6 mL) was added methanesulphonyl chloride (0.15 mL, 1.98 mmol) followed by N,N diisopropylethylamine (0.31 mL, 1.76 mmol) at 0° C. under dry reaction conditions. After 1 h the reaction was quenched by the addition of water (1×40 mL) and the product was extracted with diethyl ether (3×30 mL). The combined etheral fractions were dried over MgSO₄, filtered and concentrated to afford a white solid. The product was purified by flash column chromatography (stationary phase; silica gel 230-400 mesh, mobile phase; 1:1, hexane/ethyl acetate). All homogenous fractions were collected and the solvent was evaporated to afford the product 52.15 as a white solid (390 mg, 86%). The mesylate 52.15 (390 mg, 1.01 mmol) was dissolved in DMF (5 mL) at room temperature. Sodium azide (1.31 g, 20.2 mmol) was added to the stirred solution. The reaction mixture was heated to 80° C. After 30-6 h the reaction was quenched by the addition of water (1×50 mL) and the product was extracted with diethyl ether (3×30 mL). The combined etheral fractions were dried over MgSO₄, filtered and concentrated to afford a white solid. The product was purified by flash column chromatography (stationary phase; silica gel 230-400 mesh, mobile phase; 3:1, hexane/ethyl acetate). All homogenous fractions were collected and the solvent was evaporated to afford the product 52.16 as a colourless oil (300 mg, 89%) and an eliminated side product (10 mg, 3%).

52.16 ¹H NMR (CDCl₃, 400 MHz) δ_{H} ppm: 1.25-1.88 (2H, m, major & minor diastereomers, CH₂), 2.11 (3H, s, major diastereomer, COCH₃), 2.22 (3H, s, minor diastereomer, COCH₃), 2.27-2.39 (2H, m, major & minor diastereomers, CH₂), 2.77-3.40 (3H, m, major & minor diastereomers, CH & CH₂), 3.80 (3H, major diastereomer OMe), 3.81 (3H, s, minor diastereomer, OMe), 3.85 (6H, s, minor diastereomer, 2×OMe), 3.87 (6H, s, major diastereomer, 2×OMe), 3.90 (1H, m, major & minor diastereomers, CH), 6.66 (1H, s, major diastereomer, ArH), 6.72 (1H, s, minor diastereomer, ArH)

¹³C NMR (CDCl₃, 400 MHz) δ_{c} ppm: 19.61, 19.91 (CH₂), 20.73, 20.75 (COCH₃), 31.88, 32.44 (CH₂), 36.93, 39.09 (CH₂), 55.54 (OMe), 58.93 (CHN₃), 60.34, 60.38 (OMe), 60.92, 60.95 (OMe), 69.81 (CH), 103.02 (ArCH), 123.95, 126.49 (ArC), 133.92, 134.98 (ArC), 140.95, 141.66 (ArC), 150.63, 150.69 (ArC), 150.92, 151.18 (ArC), 169.21, 169.27 (C═O)

νₘₐₓ (DCM)/cm⁻¹ 2935.7, 2093.6, 1739.1, 1600.4, 1235.8, 1120.3

Eliminated Side Product ¹H NMR (CDCl₃, 400 MHz) δ_{H} ppm: 2.16 (2H, m, CH₂), 2.76, 3.02 (2H, m, CH₂), 3.84 (3H, s, OMe), 3.87 (3H, s, OMe), 3.91 (3H, s, OMe), 4.19 (1H, m, CHN₃), 5.84 (1H, dd, J=4.0 Hz, 12.0 Hz, CHN₃CH═CH), 6.52 (1H, d, J=12.0 Hz, CHN₃CH═CH), 6.68 (1H, s, ArH)

¹³C NMR (CDCl₃, 400 MHz) δ_{c} ppm: 20.60 (CH₂), 32.18 (CH₂), 55.56 (OMe), 60.51 (CHN₃), 60.85 (2×OMe), 110.51 (ArCH), 126.93 (CH═CH), 127.71 (ArC), 130.10 (ArC), 131.97 (CH═CH), 141.61 (ArC), 150.29 (ArC), 150.59 (ArC)

νₘₐₓ (DCM)/cm^{˜}2931.4, 2093.9, 1454.9, 1241.7, 1122.8

### Synthesis of 7-azido-6,7,8,9-tetrahydro-1,2,3-trimethoxybenzo[7]annulen-5-one 34.18

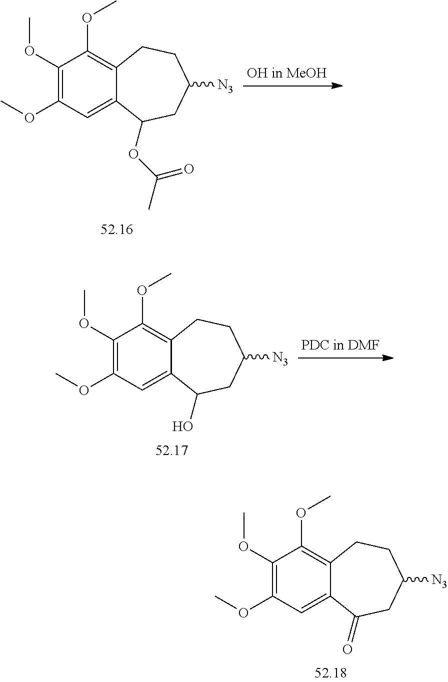 [SMILES: COc1cc2c(c(OC)c1OC)CCC(N=[N+]=[N-])CC2OC(C)=O ; COc1cc2c(c(OC)c1OC)CCC(N=[N+]=[N-])CC2O ; COc1cc2c(c(OC)c1OC)CCC(N=[N+]=[N-])CC2=O]

To a stirred solution of 52.16 (560 mg, 1.68 mmol) in methanol (10 mL) was added 2.5M aq. NaOH (20 mL) at 0° C. After 10 min the flask was removed from the ice and the temperature was allowed to increase to ambient. After an hour the reaction mixture was quenched with sat. aq. NaCl solution (1×50 mL) and the product was extracted with diethyl ether (3×40 mL). The combined etheral fractions were dried over MgSO₄, filtered and concentrated to a colourless oil. The product was not purified by flash column chromatography. The alcohol 52.17 was dissolved in DMF (6 mL) and pyridinium dichromate (1.13 g, 3.00 mmol) was added to the stirred solution at room temperature. The product was purified by flash column chromatography (stationary phase; silica gel 230-400 mesh, mobile phase; 3:1, hexane/ethyl acetate). All homogenous fractions were collected and the solvent was evaporated to afford the product 52.18 as a colourless oil (390 mg, 89%).

¹H NMR (CDCl₃, 400 MHz) δ_{H} ppm: 1.98, 2.10 (2H, m, CH₂), 2.88, 3.11 (4H, m, 2×CH₂), 3.82 (3H, s, OMe), 3.85 (3H, s, OMe), 3.89 (3H, s, OMe), 3.98 (1H, m, CHN₃), 7.11 (1H, s, ArH)

¹³C NMR (CDCl₃, 400 MHz) δ_{c} ppm: 20.68 (CH₂), 31.70 (CH₂), 45.99 (CH₂), 55.49 (OMe), 56.13 (CHN₃), 60.39 (OMe), 60.74 (OMe), 107.02 (ArCH), 129.01 (ArC), 133.16 (ArC), 145.50 (ArC), 150.61 (ArC), 151.29 (ArC), 198.02 (C═O)

νₘₐₓ (DCM)/cm⁻¹ 2935.7, 2101.3, 1675.5, 1588.5, 1486.5, 1325.25, 1095.2

### Synthesis of tert-butyl 6,7,8,9-tetrahydro-1,2,3-trimethoxy-5-oxo-5H-benzo[7]annulen-7-yl carbamate 52.19

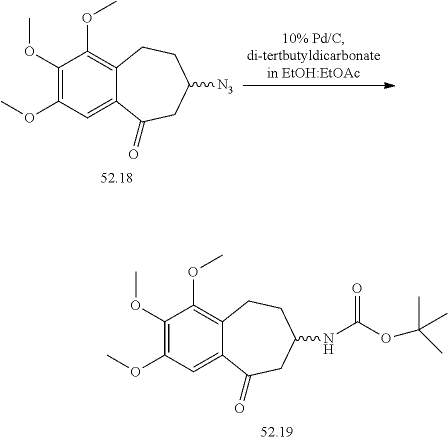 [SMILES: COc1cc2c(c(OC)c1OC)CCC(N=[N+]=[N-])CC2=O ; COc1cc2c(c(OC)c1OC)CCC(NC(=O)OC(C)(C)C)CC2=O]

To a stirred solution of 52.18 (350 mg, 1.20 mmol) in a 1:1 mixture of ethanol and ethyl acetate (6 mL) was added di-tert-butyl dicarbonate (525 mg, 2.40 mmol) and 10% Pd/C (catalytic amount). The mixture was stirred at room temperature under an atmosphere of hydrogen (balloon). The reaction was monitored by TLC. After sixteen h the Pd/C was removed from the reaction mixture by filtration using DCM (200 mL). The solvent was removed from the flask under vacuum and the resulting residue was purified by flash column chromatography (stationary phase; silica gel 230-400 mesh, mobile phase; 2:1, hexane/ethyl acetate). All homogenous fractions were collected and the solvent was evaporated to afford 52.19 as a yellow solid (340 mg, 78%).

¹H NMR (CDCl₃, 400 MHz) δ_{H} ppm: 1.38 (9H, s C(CH₃)₃), 1.54 (1H, s, br, CH₂), 2.25 (1H, s, br, CH₂), 2.74-3.03 (4H, m, 2×CH₂), 3.78 (3H, s, OMe), 3.81 (3H, s, OMe), 3.87 (3H, s, OMe), 4.05 (1H, m, CH), 4.82 (1H, d, J=7.5 Hz, NH), 7.04 (1H, s, ArH)

¹³C NMR (CDCl₃, 400 MHz) δ_{c} ppm: 21.45 (CH₂), 27.86 (C(CH₃)₃), 32.66 (CH₂), 45.96 (CH), 47.15 (CH₂), 55.46 (OMe), 60.36 (OMe), 60.71 (OMe), 79.03 (C(CH₃)₃), 106.84 (ArCH), 128.93 (ArC), 133.59 (ArC), 145.27 (ArC), 150.57 (ArC), 151.22 (ArC), 154.53 (C═O), 200.32 (C═O)

νₘₐₓ (DCM)/cm⁻¹ 3352.6, 2937.8, 1711.3, 1678.6, 1487.5, 1325.9, 1168.5

HRMS: calculated 388.1736, found 388.1747, molecular formula (C₁₉H₂₇NO₆Na).

Formation of the triflate of 52.20

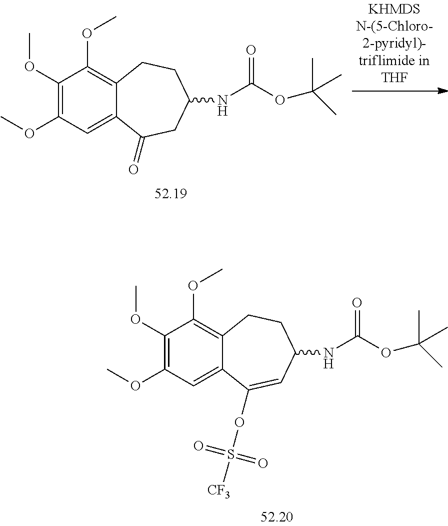

### Synthesis

To a dry three-necked round bottom flask containing 52.19 (20 mg, 0.05 mmol) dissolved in anhydrous THF (2 mL) was added KHMDS (0.5M in toluene) (0.14 mL 0.7 mmol) under dry reaction conditions at 0° C. The resultant suspension was allowed to stir at this temperature for 2 h and a solution of N,N-bis-(trifluoromethylsulfonyl)amino-5-chloropyridine (51 mg, 0.13 mmol) in dry THF (2 mL) was added. The reaction was allowed to stir for an additional 3 h at this temperature. The reaction was quenched by the addition of water (1×50 mL) and extracted with diethyl ether (3×50 mL). The combined organic fractions were dried over MgSO₄, filtered and dried under vacuum. The residue was purified by flash column chromatography (stationary phase; silica gel 230-400 mesh, mobile phase; 6:1, hexane/ethyl acetate) to yield 52.20 as a colourless oil (25 mg, 100%).

¹H NMR (CDCl₃, 400 MHz) δ_{H} ppm: 1.46 (9H, s C(CH₃)₃), 2.03 (1H, m, CH₂), 2.31 (1H, m, CH₂), 2.44 (1H, m, CH₂), 3.07 (1H, m, CH₂), 3.87 (6H, s, 2×OMe), 3.93 (3H, s, OMe), 4.19 (1H, m, CH), 4.71 (1H, m, NH), 6.05 (1H, d, J=5.0 Hz, C═CH), 6.83 (1H, s, ArH)

¹³C NMR (CDCl₃, 400 MHz) δ_{c} ppm: 21.04 (CH₂), 27.87 (C(CH₃)₃), 38.21 (CH₂), 47.33 (CH), 55.57 (OMe), 60.48 (OMe), 61.09 (OMe), 79.67 (C(CH₃)₃), 105.50 (ArCH), 124.67 (C═CH), 126.72 (ArC), 127.32 (ArC), 143.21 (ArC), 144.13 (ArC), 150.64 (ArC), 151.46 (ArC), 154.47 (C═O)

¹⁹F NMR (CDCl₃, 400 MHz) δ_{c} ppm: −74.39 (CF₃)

νₘₐₓ (DCM)/cm⁻¹ 3350.6, 2933.8, 1689.2, 1417.8, 1120.4, 870.7

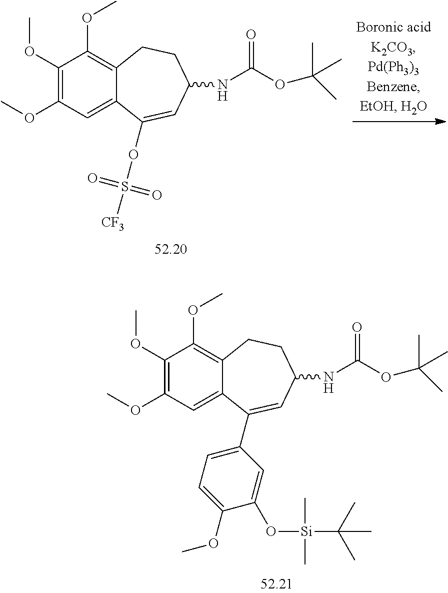

To a flask containing the triflate 52.20 (80 mg, 0.16 mmol) was added the boronic acid 1.15 (54 mg, 0.19 mmol), K₂CO₃ (66 mg, 0.48 mmol), and Pd(Ph₃)₄ (9 mg, 0.008 mmol). The mixture was dissolved in benzene, ethanol and water (3:1:1. 1.6 mL). The resulting mixture was refluxed for 1 h. The reaction was quenched by the addition of water (1×20 mL) and the product was extracted with diethyl ether (3×30 mL). The organic fractions were combined, dried over MgSO₄, filtered and concentrated in vacuo. The resulting residue was purified by flash column chromatography (stationary phase; silica gel 230-400 mesh, mobile phase; 4:1, hexane/ethyl acetate). All homogenous fractions were collected and the solvent was evaporated to afford 52.21 as a colourless oil (90 mg, 96%).

¹H NMR (CDCl₃, 400 MHz) δ_{H} ppm: 0.15 (3H, s, Si(CH₃)), 0.16 (3H, s, Si(CH₃)), 0.99 (9H, s C(CH₃)₃), 1.46 (9H, s, C(CH₃)₃), 2.06 (1H, m, CH₂), 2.33 (1H, m, CH₂), 2.45 (1H, m, CH₂), 3.06 (1H, m, CH₂), 3.68 (3H, s, OMe), 3.83 (3H, s, OMe), 3.93 (6H, s, 2×OMe), 4.04 (1H, m, CH), 4.70 (1H, m, NH), 6.02 (1H, d, J=5.5 Hz, C═CH), 6.36 (1H, s, ArH {A-ring}), 6.80 (3H, m, 3×ArH {C-ring})

¹³C NMR (CDCl₃, 400 MHz) δ_{c} ppm: −4.99 (Si(CH₃)₂), 17.99 (C(CH₃)₃), 21.79 (CH₂), 25.27 (C(CH₃)₃), 27.94 (C(CH₃)₃), 41.96 (CH₂), 47.33 (CH), 55.02 (OMe), 55.44 (OMe), 60.40 (OMe), 61.15 (OMe), 79.67 (C(CH₃)₃), 108.57 (ArCH), 111.10 (ArCH), 120.03 (ArCH), 121.06 (ArCH), 126.86 (ArC), 128.93 (ArC), 133.72 (C═CH), 140.95 (ArC), 144.11 (ArC), 150.09 (ArC), 150.30 (ArC), 150.75 (C═O)

νₘₐₓ (DCM)/cm⁻¹ 3367.7, 2931.0, 1714.3, 1508.5, 1263.7

HRMS: calculated 608.3020, found 608.3039, molecular formula (C₃₂H₄₇NO₇SiNa).

### Synthesis of tert-butyl (Z)-6,7-dihydro-9-(3-hydroxy-4-methoxyphenyl)-2,3,4-trimethoxy-5H-benzo[7]annulen-7-ylcarbamate 52.22

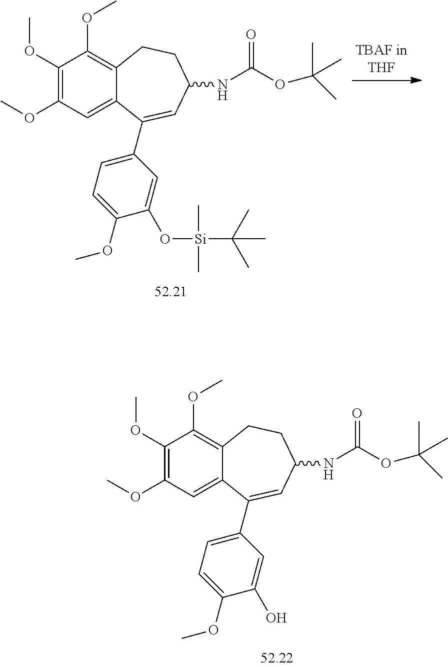

To a stirred solution of 52.21 (50 mg, 0.09 mmol) in THF (1 mL) was added 1M TBAF (1 mL, 1.00 mmol) at 0° C. After 1 h the reaction was quenched by the addition of sat. aq. NaCl (1×30 mL) and the product was extracted with diethyl ether (3×30 mL). The etheral extracts were combined, dried over MgSO₄ and filtered. The organic fractions were applied directly to a flash column, without prior concentration of the solution in vacuo. The product was purified by flash column chromatography (stationary phase; silica gel 230-400 mesh, mobile phase; 4:1, hexane/ethyl acetate). All homogenous fractions were collected and the solvent was evaporated to afford 52.22 as a colourless oil (40 mg, 100%).

¹H NMR (CDCl₃, 400 MHz) δ_{H} ppm: 1.46 (9H, s, C(CH₃)₃), 1.94 (1H, m, CH₂), 2.33 (1H, m, CH₂), 2.47 (1H, m, CH₂), 3.07 (1H, m, CH₂), 3.69 (3H, s, OMe), 3.92 (9H, s, 3×OMe), 4.13 (1H, m, CH), 4.70 (1H, m, NH), 5.64 (1H, s, br, OH), 6.05 (1H, d, J=5.5 Hz, C═CH), 6.38 (1H, s, ArH {A-ring}), 6.80 (2H, s, 2×ArH {C-ring}), 6.91 (1H, s, ArH {C-ring})

¹³C NMR (CDCl₃, 400 MHz) δ_{c} ppm: 21.77 (CH₂), 27.97 (C(CH₃)₃), 29.26 (CH₂), 48.62 (CH), 55.53 (OMe), 55.56 (OMe), 60.42 (OMe), 61.14 (OMe), 78.95 (C(CH₃)₃), 108.66 (ArCH), 109.78 (ArCH), 113.82 (ArCH), 119.43 (ArCH), 126.86 (ArC), 129.44 (C═CH), 134.32 (ArC), 134.64 (ArC), 139.50 (ArC), 141.01 (ArC), 144.79 (ArC), 145.70 (ArC), 150.31 (ArC), 150.81 (C═O)

νₘₐₓ (DCM)/cm⁻¹ 3362.8, 2931.2, 1695.3, 1277.7, 1169.4

### Synthesis of 52.23

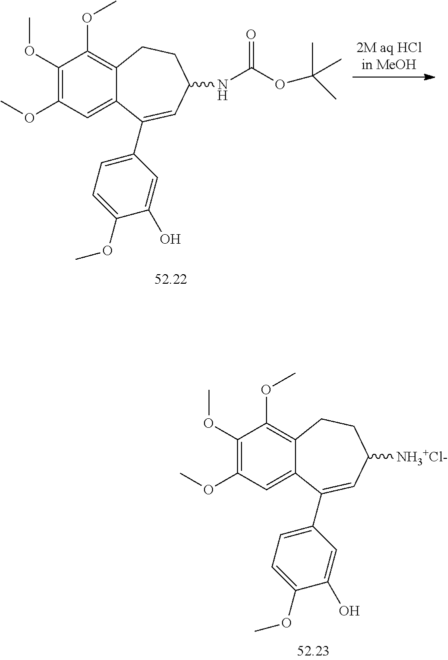

### Synthesis A

To a stirred solution of 52.22 (40 mg, 0.09 mmol) in methanol (1 mL) was added 2M aq. HCl (1 mL). The stirred solution was heated to 40° C. for 30 min. The solvent was removed from the flask under vacuum at 80° C. The resulting HCl salt 52.23 was dried in vacuo for several days.

### Synthesis B

To a stirred solution of 52.22 (40 mg, 0.07 mmol) in methanol (1 mL) was added 2M aq. HCl (1 mL) followed by THF (1 mL). The resulting mixture was heated to 40° C. for 30 min. The mixture was allowed to cool to room temperature and water (1×10 mL) was added to the flask. The impurities were extracted with diethyl ether (3×20 mL) and the aqueous layer was retained. The water was removed from the flask under vacuum with the aid of methanol (80 mL). The resulting HCl salt 52.23 was dried in vacuo for several days.

¹H NMR (MeOD, 400 MHz) δ_{H} ppm: 2.24 (1H, m, CH₂), 2.39 (1H, m, CH₂), 2.54 (1H, m, CH₂), 3.17 (1H, m, CH₂), 3.55 (1H, m, CH), 3.68 (3H, s, OMe), 3.88 (6H, s, 2×OMe), 3.92 (3H, s, OMe), 6.13 (1H, d, J=5.5 Hz, C═CH), 6.43 (1H, s, ArH {A-ring}), 6.77 (2H, s, 2×ArH {C-ring}), 6.93 (1H, d, J=8.0 Hz, ArH {C-ring})

¹³C NMR (MeOD, 400 MHz) δ_{c} ppm: 20.91 (CH₂), 38.94 (CH₂), 48.73 (CH), 54.61 (OMe), 54.66 (OMe), 59.45 (OMe), 60.32 (OMe), 108.46 (ArCH), 110.60 (ArCH), 114.20 (ArCH), 119.03 (ArCH), 120.67 (C═CH), 132.63 (ArC), 134.06 (ArC), 141.53 (ArC), 142.87 (ArC), 145.65 (ArC), 147.59 (ArC), 150.40 (ArC), 151.36 (ArC)

νₘₐₓ (KBr)/cm⁻¹ 13400.3, 2937.0, 1593.9, 1509.4, 1252.5, 1116.7

Melting point: 139-144° C.

### Synthesis of 52.24

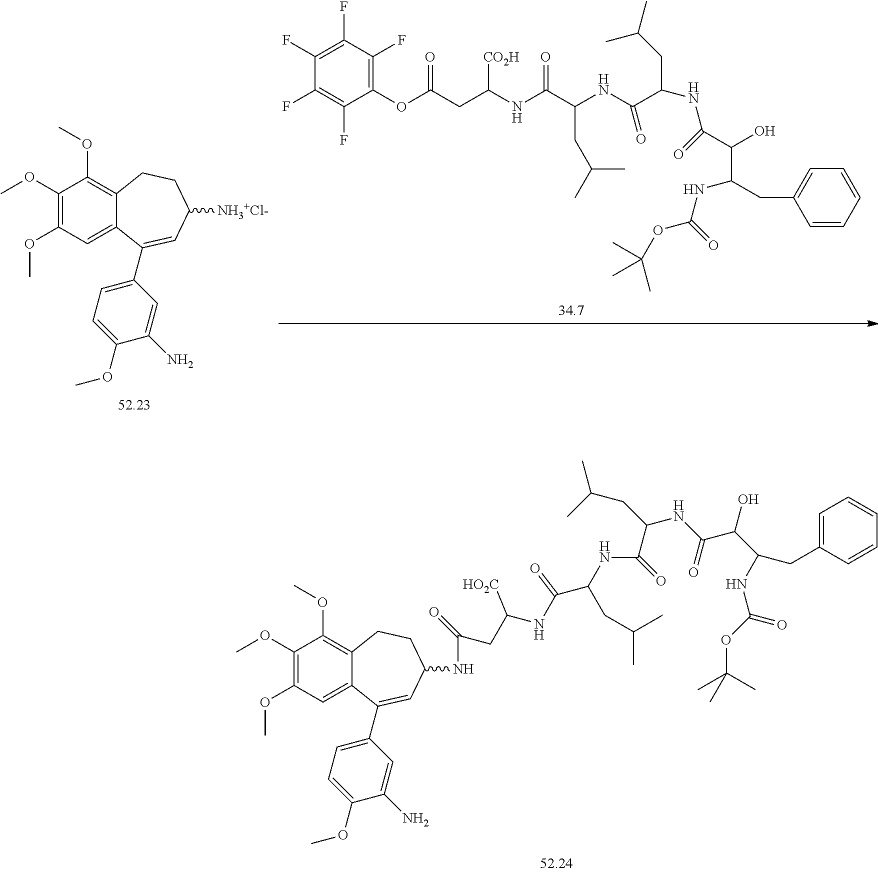

To a stirred solution of 52.23 (30 mg, 0.07 mmol) in anhydrous DCM (5 mL) was added 52.7 (60 mg, 0.09 mmol) followed by Et₃N (0.02 mL, 0.14 mmol) at 0° C. under dry reaction conditions. After 10 min the reaction temperature was allowed to increase to ambient and the reaction was allowed to proceed for a further 2 h. The reaction was quenched by the addition of water (1×20 mL) and the product was extracted with diethyl ether (3×30 mL). The combined organic fractions were washed with 2.5M aq. NaOH (3×20 mL). The basic aqueous fractions were combined and acidified with 2M aq. HCl (1×100 mL). The product was extracted with diethyl ether (3×30 mL). The combined etheral fractions were dried over MgSO₄, filtered and concentrated to afford an off-white solid. The product was purified by flash column chromatography (stationary phase; silica gel 230-400 mesh, mobile phase; 1:1, hexane/ethyl acetate). All homogenous fractions were collected and the solvent was evaporated to afford the product 52.24 as colourless oil, quantitatively.

¹H NMR (CDCl₃, 400 MHz) δ_{H} ppm: 0.90 (6H, m, major & minor diastereomers, 2×CH₃ (Leu)), 1.32 (9H, s, major diastereomer, C(CH₃)₃), 1.37 (9H, s, minor diastereomer, C(CH₃)₃), 1.65 (3H, m, major & minor diastereomers, CH& CH₂ (Leu)), 2.00 (2H, m, major & minor diastereomers, CH₂), 2.40 (2H, m, major & minor diastereomers, CH₂), 2.90 (4H, m, major & minor diastereomers, 2×CH₂), 3.63 (3H, m, major & minor diastereomers, OMe), 3.91 (9H, m, major & minor diastereomers, 3×OMe), 4.18 (2H, m, major & minor diastereomers, 2×CH), 4.66 (3H, m, major & minor diastereomers, 3×CH), 5.16 (1H, m, major & minor diastereomers, NH), 6.04 (1H, m, major & minor diastereomers, C═CH), 6.35 (1H, m, major & minor diastereomers, ArH {A-ring}), 6.74 (3H, m, major & minor diastereomers, 3×ArH {C-ring}), 7.20 (5H, m, major & minor diastereomers, 5×ArH(AHPA)), 7.53 (3H, m, major & minor diastereomers, 3×NH)

¹³C NMR (CDCl₃, 400 MHz) δ_{c} ppm: 21.18, 21.36 (CH₃(Leu)), 22.13, 22.69 (CH₂ (Leu)), 22.27, 22.60 (CH₃(Leu)), 24.66 (CH (Leu)), 28.02, 28.22 (C(CH₃)₃), 31.93, 32.22 (CH₂), 33.64, 33.73 (CH₂), 37.10, 37.39 (CH₂), 40.58, 40.98 (CH₂), 48.53, 48.69 (CH), 48.77 (CH), 49.53, 51.56 (CH), 55.79 (2×OMe), 60.69 (OMe), 61.39 (OMe), 77.23 (CH), 80.10, 80.50 (C(CH₃)₃), 109.12, 109.19 (ArCH), 110.45, 110.59 (ArCH), 114.27 (ArCH), 120.23 (ArCH), 125.05 (ArCH), 126.51 (ArCH), 127.00 (C═CH), 128.45 (2×ArCH), 129.31 (ArCH), 135.30 (ArC), 136.28 (ArC), 138.72 (ArC), 141.03 (ArC), 150.85 (ArC)

νₘₐₓ (DCM)/cm⁻¹: 3325.2, 2929.4, 1651.0, 1516.5, 1248.6

HRMS: calculated 899.4055, found 899.4025, molecular formula (C₄₆H₆₀N₄O₁₃Na).

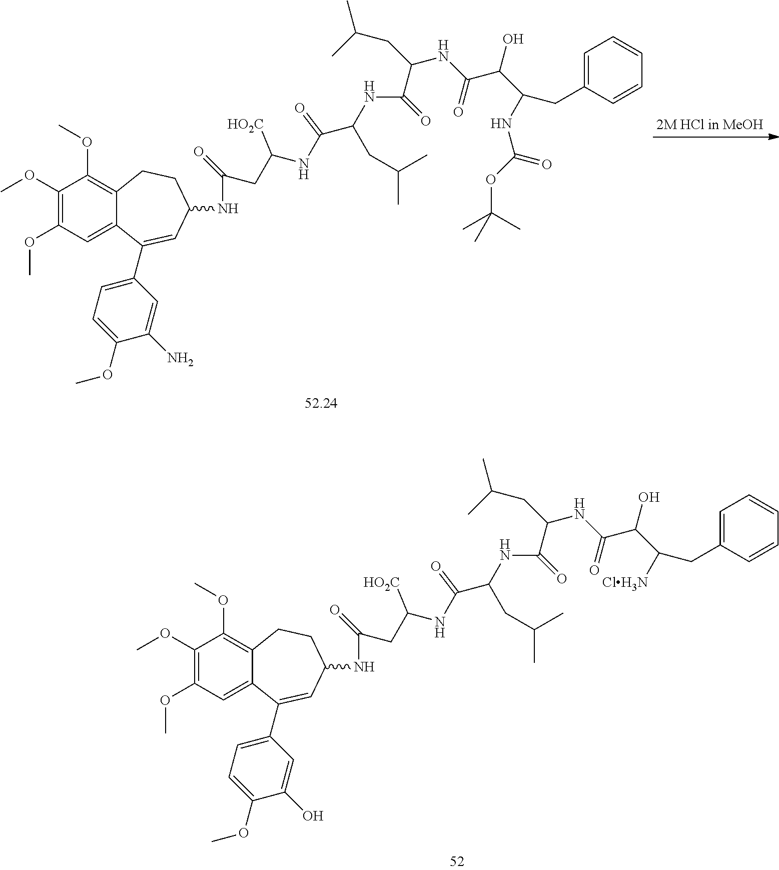

To a stirred solution of 52.24 (40 mg, 0.04 mmol) in methanol (1 mL) was added 2M aq. HCl (1 mL) followed by THF (1 mL). The resulting mixture was heated to 40° C. for 30 min. The mixture was allowed to cool to room temperature and water (1×10 mL) was added to the flask. The impurities were extracted with diethyl ether (3×20 mL) and the aqueous layer was retained. The water was removed from the flask under vacuum with the aid of methanol (80 mL). The resulting HCl salt 52 was dried in vacuo for several days.

¹H NMR (MeOD, 400 MHz) δ_{H} ppm: 0.96 (6H, m, major & minor diastereomers, 2×CH₃(Leu)), 1.67 (3H, m, major & minor diastereomers, CH & CH₂(Leu)), 2.08 (2H, major & minor diastereomers, CH₂), 2.30 (2H, m, major & minor diastereomers, CH₂), 2.74-3.08 (4H, m, major & minor diastereomers, 2×CH₂), 3.65 (3H, m, major diastereomer, OMe), 3.70 (3H, m, minor diastereomer, OMe), 3.89 (9H, s, major diastereomer, 3×OMe), 3.91 (9H, s, minor diastereomer, OMe), 4.10 (1H, m, major & minor diastereomers, CH), 4.18 (1H, m, major & minor diastereomers, CH), 4.28 (1H, m, major & minor diastereomers, CH), 4.40 (1H, m, major & minor diastereomers, CH), 4.64 (1H, m, major & minor diastereomers, CH), 6.05 (1H, d, J=3.5 Hz, minor diastereomer, C═CH), 6.08 (1H, d, J=3.5 Hz, major diastereomer, C═CH), 6.43 (1H, m, major & minor diastereomers, ArH {A-ring}), 6.76 (2H, m, minor diastereomer, 2×ArH {C-ring}), 6.81 (2H, m, major diastereomer, 2×ArH {C-ring}), 6.95 (1H, m, major & minor diastereomers, ArH {C-ring}), 7.24-7.42 (5H, m, major & minor diastereomers, 5×ArH (AHPA))

¹³C NMR (MeOD, 400 MHz) δ_{c} ppm: 20.89, 20.97 (CH₃(Leu)), 21.00, 21.05 (CH₂(Leu)), 21.69, 21.78 (CH₃(Leu)), 24.34 (CH (Leu)), 34.84, 34.88 (CH₂), 36.40, 36.45 (CH₂), 39.99, 40.07 (CH₂), 44.26 (CH₂), 49.31, 49.40 (CH), 51.62, 51.68 (CH), 52.20, 52.25 (CH), 54.51, 54.55 (CH), 54.65, 55.25 (OMe), 55.28, 55.33 (OMe), 60.33 (OMe), 61.20 (OMe), 68.51, 68.64 (CH), 109.08 (ArCH), 111.31, 111.36 (ArCH), 114.48, 114.52 (ArCH), 119.62, 119.65 (ArCH), 127.23, 127.79 (ArCH), 127.38, 127.41 (C═CH), 128.77, 128.91 (2×ArCH), 128.98, 129.04 (2×ArCH), 133.89, 133.91 (ArC), 134.90 (ArC), 135.48, 135.51 (ArC), 140.03, 140.05 (ArC), 141.03 (ArC), 145.32, 145.34 (ArC), 147.39 (ArC), 150.30 (ArC), 150.97, 151.01 (ArC), 169.99, 170.04 (C═O), 171.81, 171.85 (C═O), 172.09, 172.14 (C═O), 173.15, 173.17 (C═O)

νₘₐₓ (KBr)/cm⁻¹: 3391.6, 2930.9, 1740.5, 1652.0, 1510.0, 1440.5, 1276.9, 1112.7

Melting point: 225-230° C.

### Synthesis of Compound 53

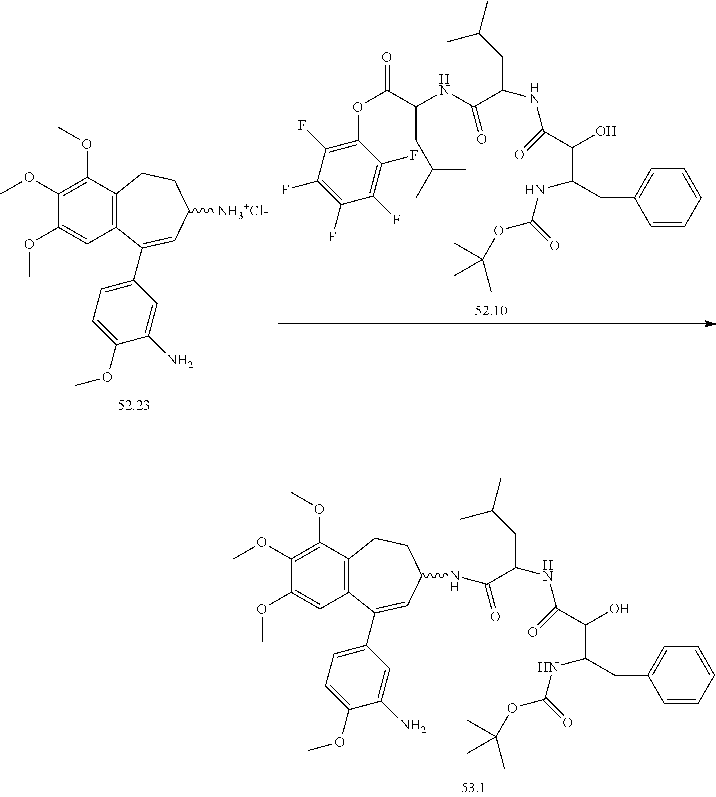

To a stirred solution of diamine (10 mg, 0.03 mmol) in anhydrous DCM (5 mL) was added 32.10 (30 mg, 0.06 mmol) followed by Et₃N (0.01 mL, 0.06 mmol) at 0° C. under dry reaction conditions. After 10 min the reaction temperature was allowed to increase to ambient and the reaction was allowed to proceed for a further 2 h. The reaction was quenched by the addition of water (1×20 mL) and the product was extracted with diethyl ether (3×30 mL). The combined organic fractions were dried over MgSO₄, filtered and concentrated to afford an off-white solid. The product was purified by flash column chromatography (stationary phase; silica gel 230-400 mesh, mobile phase; 1:1, hexane/ethyl acetate). All homogenous fractions were collected and the solvent was evaporated to afford the product 53.1 as a colourless oil, quantitatively.

¹H NMR (CDCl₃, 400 MHz) δ_{H} ppm: 0.90 (6H, m, major & minor diastereomers, 2×CH₃ (Leu)), 1.28 (9H, s, major diastereomer, C(CH₃)₃), 1.39 (9H, s, minor diastereomer, C(CH₃)₃), 1.59 (1H, m, major & minor diastereomers, CH(Leu)), 1.71 (2H, m, major & minor diastereomers, CH₂ (Leu)), 2.38 (2H, m, major & minor diastereomers, CH₂), 3.00 (2H, m, major diastereomer, CH₂), 3.16 (2H, m, minor diastereomer, CH₂), 3.67 (3H, s, major & minor diastereomers, OMe), 3.89 (3H, s, major & minor diastereomers, OMe), 3.91 (3H, s, major & minor diastereomers, OMe), 3.93 (3H, s, major & minor diastereoemers, OMe), 4.14 (1H, m, major & minor diastereomers, CH), 4.19 (1H, m, major & minor diastereomers, CH), 4.28 (1H, m, major & minor diastereomers, CH), 4.44 (1H, m, major & minor diastereomers, CH), 5.01 (1H, m, NH), 5.68 (1H, m, major & minor diastereomers, CH), 5.96 (1H, m, minor diastereomer, C═CH), 6.03 (1H, d, J=6.0 Hz, major diastereomer, C═CH), 6.34 (1H, s, minor diastereomer, ArH {A-ring}), 6.36 (1H, s, major diastereomer, ArH {A-ring}), 6.50 (1H, m, major & minor diastereomers, NH), 6.77 (2H, m, major & minor diastereomers, 2×ArH), 6.87 (1H, m, major & minor diastereomers, ArH), 7.22 (1H, m, major & minor diastereomers, NH), 7.27 (5H, m, major & minor diasteroemers, 5×ArH)

¹³C NMR (CDCl₃, 400 MHz) δ_{c} ppm: 21.23, 21.72 (CH₃ (Leu)), 22.20, 22.56 (CH₃ (Leu)), 22.68, 24.15 (CH (Leu)), 27.71, 29.16 (C(CH₃)₃), 31.44 (CH₂), 35.91, 36.61 (CH₂), 40.21, 40.35 (CH), 41.03, 41.06 (CH), 47.56, 47.59 (CH), 50.91, 50.99 (CH), 55.28, 55.32 (OMe), 55.51, 55.54 (OMe), 60.38, 60.40 (OMe), 61.09, 61.11 (OMe), 73.65 (CHOH), 80.12 (C(CH₃)₃), 108.72, 108.76 (ArCH), 109.80, 109.91 (ArCH), 113.84, 113.91 (ArCH), 119.47, 119.52 (ArCH), 126.17 (ArCH), 126.84, 126.86 (ArCH), 127.79 (ArCH), 128.00, 128.11 (2×ArCH), 128.81 (ArCH), 134.17, 134.25 (ArC), 134.57, 134.61 (ArC), 137.50 (ArC), 140.24 (ArC), 141.07, 141.11 (ArC), 144.81 (ArC), 145.79 (ArC), 150.35 (ArC), 150.86 (ArC), 157.04 (C═O), 170.26 (C═O), 172.49 (C═O)

νₘₐₓ (DCM)/cm⁻¹ 33.09.8, 2928.1, 1647.9, 1508.3, 1367.0, 1168.9

### Synthesis of 53

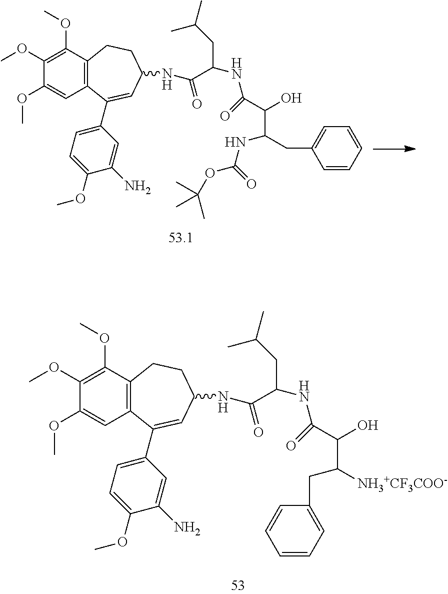

To a stirred solution of 35.1 (20 mg, 0.03 mmol) in methanol (1 mL) was added 2M aq. HCl (1 mL) followed by THF (1 mL). The resulting mixture was heated to 40° C. for 30 min. The mixture was allowed to cool to room temperature and water (1×10 mL) was added to the flask. The impurities were extracted with diethyl ether (3×20 mL) and the aqueous layer was retained. The water was removed from the flask under vacuum with the aid of methanol (80 mL). The resulting HCl salt 35 was dried in vacuo for several days.

¹H NMR (MeOD, 400 MHz) δ_{H} ppm: 1.01 (6H, m, major & minor distereomers, 2×CH₃ (Leu)), 1.71 (3H, m, major & minor diastereomers, CH & CH₂ (Leu)), 2.32 (2H, m, major & minor diastereomers, CH₂), 2.94 (2H, m, major & minor diatereomers, CH₂), 3.11 (2H, m, major & minor diastereomers, CH₂), 3.67 (3H, m, major & minor diastereomers, OMe), 3.87 (9H, s, major diastereomer, 3×OMe), 3.90 (9H, s, minor diastereomers, 3×OMe), 4.16 (1H, m, major & minor diastereomers, CH), 4.39 (1H, m, major & minor diasteromers, CH), 4.48 (1H, m, major & minor diasteromers, CH), 6.03 (1H, d, J=3.8 Hz, minor diastereomer, C═CH), 6.10 (1H, d, J=3.8 Hz, major diasteromer, C═CH), 6.40 (1H, s, major diastereomer, ArH {A-ring}), 6.41 (1H, s, minor diastereomer, ArH {A-ring}), 6.72 (2H, m, major & minor diastereomers, 2×ArH {C-ring})), 6.88 (1H, m, major & minor diastereomers, ArH {C-ring}), 7.30 (5H, m, major & minor diastereomers, 5×ArH)

¹³C NMR (MeOD, 400 MHz) δ_{c} ppm: 20.49, 20.53 (CH₃ (Leu)), 21.57, 21.67 (CH₃ (Leu)), 24.50, 24.55 (CH (Leu)), 31.50 (CH₂), 34.86, 34.92 (CH₂), 39.57, 39.67 (CH), 40.39, 40.48 (CH), 50.90, 51.36 (CH), 51.62, 51.70 (CH), 52.11, 52.14 (CH), 54.14, 54.84 (OMe), 55.86, 55.96 (OMe), 54.97, 55.44 (OMe), 59.73, 60.68 (OMe), 68.33, 68.40 (CH), 108.81, 108.84 (ArCH), 110.92, 110.96 (ArCH), 114.48 (ArCH), 119.07 (ArCH), 127.02, 127.05 (ArCH), 127.09, 127.18 (C═CH), 127.31 (ArCH), 128.57, 128.60 (2×ArCH), 128.94, 128.96 (ArCH), 133.94, 133.97 (ArC), 135.04, 135.14 (ArC), 135.15, 135.27 (ArC), 140.48, 140.60 (ArC), 141.35 (ArC), 145.79 (ArC), 147.35, 147.38 (ArC), 150.56, 150.59 (ArC), 151.25 (ArC), 172.26 (C═O), 173.09 (C═O)

νₘₐₓ (KBr)/cm⁻¹ 33.09.8, 2928.1, 1647.9, 1508.3, 1367.0, 1168.9

Melting point: 195-200° C.

### Synthesis of Compound 54

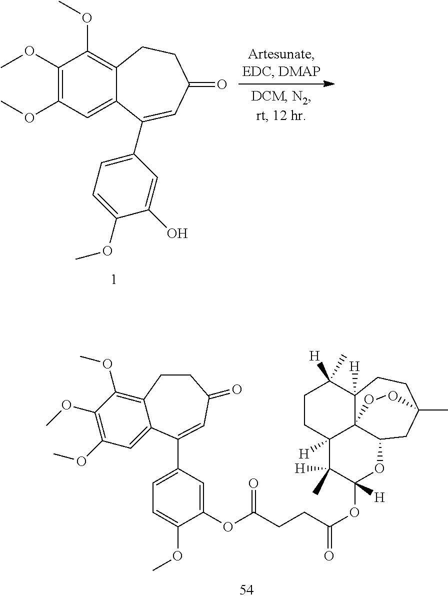

Under an atmosphere of N₂, a solution of 9-(3-hydroxy-4-methoxyphenyl)-2,3,4-trimethoxy-5H-benzo[7]annulen-7(6H)-one (0.0232 g, 0.062 mmol) was dissolved in dry DCM (2 mL). To this solution was added Artesunate (0.0240 g, 0.062 mmol) in DCM, followed by EDC (0.0144 g, 0.075 mmol) in DCM (1 mL) and finally by a solution of DMAP (0.031 g, 0.250 mmol) in DCM (1 mL). The mixture was left to stir under these conditions for 12 hr. Extraction between water (20 mL) and ether (3×20 mL) afforded the product. The combined ether layers were dried using MgSO4 and concentrated in vacuo to yield the product in crude form. Following column chromatography (Hexane/EtOAc 3:1), the desired compound 54 was isolated as a white solid.

Yield: 0.027 g, 0.036 mmol, 59.3%

¹H NMR (CDCl₃, 400 MHz) δ_{H} ppm: 0.84 (d, J=7 Hz, 3H, CH₃), 0.97 (d, J=6 Hz, 3H, CH₃), 1.32-1.41 (m, 4H), 1.43 (s, 3H, CH3), 1.45-1.5 (m, 2H), 1.59-1.65 (m, 2H), 1.70-1.79 (m, 3H), 1.86-1.92 (m, 1H), 1.97-2.02 (m, 2H), 2.38 (td, J₁=14.1 Hz, J₂=4 Hz, 1H), 2.53-2.60 (m, 1H), 2.72 (dd, J₁=7 Hz, J₂=4.5 Hz, 2H, CH₂), 2.85 (q, J=7 Hz, 2H, CH₂) 2.90-3.02 (m, 2H, CH₂), 3.14 (t, J=6 Hz, 2H, CH₂) 3.63 (s, 3H, OCH₃), 3.88 (s, 3H, OCH₃), 3.90 (s, 3H, OCH₃), 3.95 (s, 3H, OCH₃), 5.45 (s, 1H, O—CH—O), 5.82 (d, J=10 Hz, 1H), 6.37 (s, 1H), 6.39 (s, 1H), 6.98 (d, J=8.5 Hz, 1H), 7.01 (d, J=2 Hz, 1H).

¹³C NMR (CDCl₃, 100.71 MHz) δ_{c} ppm: 11.6 (CH₃), 13.7 (CH₃), 19.7 (CH₂), 19.8 (CH₃), 21.5 (CH₂), 24.1 (CH₂), 25.5 (CH₃), 28.1 (CH₂), 28.7 (CH₂), 31.5 (CH), 33.6 (CH₂), 35.7 (CH₂), 36.8 (CH), 44.7 (CH), 45.2 (CH₂), 51.1 (CH₂), 55.6 (2×CH₃), 60.5 (CH₃), 61.0 (CH₃), 79.7 (Q), 91.05 (CH), 91.8 (CH), 104.0 (Q), 111.2 (CH), 111.5 (CH), 123.6 (CH), 127.1 (CH), 127.9 (CH), 128.7 (Q), 131.6 (Q), 134.6 (Q), 138.7 (Q), 142.8 (Q), 149.5 (Q), 150.2 (Q), 150.7 (Q), 151.3 (Q), 169.8 (Q), 170.4 (Q), 203.6 (Q, C═O).

MS (+ESI): Calculated Mass 734.3302. Found 759.3032 (M+Na)⁺.

### Synthesis of Compound 55

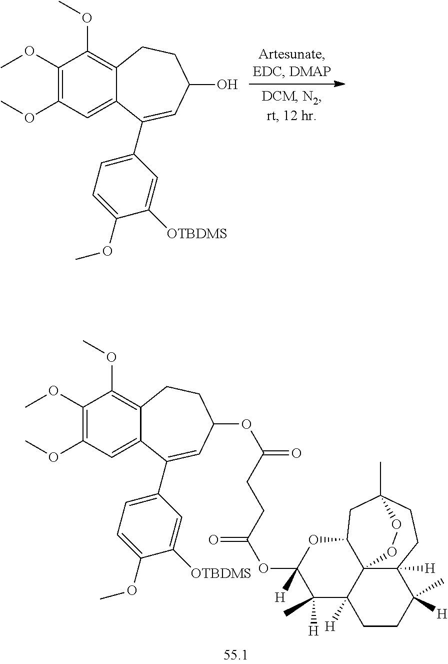 [SMILES: COc1ccc(C2=CC(O)CCc3c2cc(OC)c(OC)c3OC)cc1O[Si](C)(C)C(C)(C)C ; COc1ccc(C2=CC(OC(=O)CCC(=O)O[C@@H]3O[C@@H]4C[C@@]5(C)CC[C@H]6[C@H](C)CC[C@@H]([C@H]3C)[C@@]46OO5)CCc3c2cc(OC)c(OC)c3OC)cc1O[Si](C)(C)C(C)(C)C]

Under an atmosphere of N₂, a solution of 9-(3-((tert-butyldimethylsilyl)oxy)-4-methoxyphenyl)-2,3,4-trimethoxy-5H-benzo[7]annulen-7(6H)-one (0.045 g, 0.092 mmol) was dissolved in dry DCM (2 mL). To this solution was added artesunate (0.036 g, 0.092 mmol) in DCM (1 mL) followed by EDC (0.0213 g, 0.111 mmol) in DCM (1 mL) and finally by a solution of DMAP (0.045 g, 0.370 mmol) in DCM (1 mL). The mixture was left to stir under these conditions for 12 hr. Extraction between water (20 mL) and ether (3×20 mL) afforded the product. The combined ether layers were dried using MgSO4 and concentrated in vacuo to yield the product in crude form. Following column chromatography (Hexane/EtOAc 3:1), the desired compound 55.1 was isolated as a white solid.

¹H NMR (CDCl₃, 400 MHz) δ_{H} ppm: 0.14 (d, J=5 Hz, 6H, CH₃), 0.86 (d, J=6 Hz, 3H, CH₃), 0.97 (d, J=6 Hz, 3H, CH₃), 0.98 (s, 9H, ^{t}Bu), 1.32-1.37 (m, 4H), 1.44 (s, 3H, CH3), 1.45-1.5 (m, 3H), 1.60-1.67 (m, 2H), 1.70-1.84 (m, 2H), 1.86-1.94 (m, 1H), 2.00-2.22 (m, 3H), 2.33-2.44 (m, 2H), 2.50-2.63 (m, 2H), 2.65-2.81 (m, 2H), 3.01-3.10 (m, 1H), 3.63 (s, 3H, OCH₃), 3.82 (s, 3H, OCH₃), 3.84-3.89 (m, 1H, CHOR), 3.91 (s, 3H, OCH₃), 3.92 (s, 3H, OCH₃), 5.13-5.23 (m, 1H, O—CH—C), 5.46 (s, 1H, 0-CH-0) 6.09 (m, 1H), 6.35 (s, 1H), 6.76 (s, 1H), 6.81 (s, 1H), 6.88 (d, J=2 Hz, 1H).

MS (+ESI): Calculated Mass 852.4324. Found 875.4000 (M+Na)⁺.

The invention also provides a pharmaceutical pack or kit comprising one or more containers filled with one or more of the ingredients of the compositions of the invention. The invention is not limited to the embodiment hereinbefore described which may be varied in construction and detail with departing from the spirit of the invention.

### Experimental—Biology

### MTT Assay

The MTT assay was used to assess the anti-proliferative activity of test compounds on proliferating cells. The assay was first described by Mosmann but further slight modifications were made in order to adjust the assay to the purpose of the experiments. This is a quantitative colorimetric assay which detects living but not dead cells by generating a signal dependent on the degree of cell activation. MTT (3-(4,5-Dimethylthiazol-2-yl)-2,5-diphenyltetrazolium bromide) a yellow tetrazole, is reduced by active reductase enzyme present only in living cells, to purple formazan insoluble crystals which are then solubilised by either dimethyl sulfoxide or an acidified ethanol solution to give a coloured solution. The absorbance of this solution can be quantified by measuring at a wavelength between 500 and 600 nm using a spectrophotometer. The MTT assay is widely used and is considered a precise and rapid assay that can be used for the screening of multiple compounds [1].

### Cell Cycle Analysis

Proliferating cells are constantly dividing to produce daughter cells. The most obvious cellular structure that requires duplication and division is the cell nucleus where DNA, cell's genetic material is present. During a cell cycle there are changes in the DNA content and thus there are different cycle stages including G₀, G₁, S, G₂ and M (mitosis). The G₀ stage of the cycle is a subset of G₁ where the cells are quiescent and have limited cellular functions. Cells remain in the G₁ portion of the cycle when are not in a process of cell division and thus G₁ phase is the most predominant phase of the cycle appearing as the largest peak. In addition, during the G₁ phase synthesis of many RNA and protein molecules is taking place which are necessary for DNA synthesis. G₂ phase is a period for repair of any damaged DNA and for reorganisation of the DNA structure before entering the M phase. Cell cycle abnormalities could be a sign of cell damage, for example DNA damage causes interruption of the cell cycle at certain checkpoints such as the spindle assembly checkpoint (SAC) to prevent carcinogenesis [2].

The mitotic spindle is composed of microtubules which together with regulatory proteins assist in the activity of properly segregating replicated chromosomes. It is well established that treatment of cells with tubulin polymerization inhibitors causes a G₂/M phase arrest of the cell cycle and thus an observable increased DNA content at that stage [3-5].

For this reason, cell cycle analysis was carried out using propidium iodide (PI) staining. The first protocol was presented by Krishan in 1975 [6]. The first step involves permeabilisation of the cells' plasma membrane by fixating the cells in ethanol in order for the fluorescent DNA dye to pass through the membrane and into the cells. Ribonuclease A (RNase A) was used to eliminate any RNA present inside the cells in order to avoid creating artefacts that could affect the results. PI was used for the staining of the cells' DNA content; however other fluorescent dyes can be used instead, such as DAPI and Hoechst 33342.

Flow cytometry was used to quantify the DNA contents of the cells. This is a technique which was first introduced by Fulwyler which examines and counts cells or other microscopic particles by suspending them in a stream of fluid and passing them by an electronic detection apparatus [7]. Cells had to pass through the flow cytometer's laser where a fluorescence pulse was generated and it correlated with the amount of DNA dye present inside the cells. Cell aggregates were excluded from analysis through double discrimination.

### Immunofluorescence Staining of the Microtubule

In order to confirm that test compounds are able to interfere with cells' tubulin cytoskeleton, immunofluorescence staining was performed. The effect of test compounds on the tubulin reorganization was visualised by staining of microtubules using an α-tubulin antibody and the cell nuclei using 4′,6-diamidino-2-phenylindole (DAPI) [5].

### Analysis of Endothelial Cell Morphology

Tubulin inhibitors are not only known for their anti-mitotic effect but also for their anti-vascular activity. Treatment with such agents was previously shown to cause vascular shutdown independently of a direct cytotoxic effect on tumour cells [8]. Hence, the endothelial cell morphology was examined after treatment with test compounds at 1 h and 3 h after washout. The 1 h incubation period (after drug washout) aimed to identify any observable morphological changes of endothelial cells; however the 3 h incubation period served to indicate any signs of reversibility of this effect [9].

In vitro Angiogenesis Assay

In addition to the anti-mitotic and anti-vascular activity, tubulin targeting agents such as CA-4 exhibit anti-angiogenic effects at lower doses that do not inhibit the proliferation of endothelial cells [10]. For that reason, the angiogenic response of one of our most promising compound was evaluated using the AngioKit assay which was supplied by TCS Cellworks. AngioKit assay is a co-culture of HUVECs and matrix-producing cells which represent a better angiogenesis model in vitro [11].

### Aortic Ring Assay

The aortic ring assay is an ideal assay for testing the activity of anti-vascular, anti-angiogenic or pro-angiongenic agents. This method was originally developed and modified by Nicosia [12]. The aorta can be excised from mice or rats, cut into ˜1 mm rings and cultured in either basement membrane matrix (matrigel) or collagen gel. Following incubation for several days the rings start to generate outgrowths of branching microvessels which are lined by endothelial cells and supported by pericytes and fibroblasts. The angiogenic response of the aorta is a self-limited process regulated by endogenous growth factors including bFGF [13], VEGF [14], PDGF [15] and angiopoietins [16].

The rat aorta model is an ex vivo assay which combines advantages of both in vitro and in vivo models. The process of angiogenesis takes place in a defined culture environment which can be easily adapted to different experimental conditions according to the requirement of the study. The cells of the aortic outgrowth have not been modified by repeated passages in culture and thus newly formed microvessels reproduce the characteristics of the vasculature formed during angiogenesis in vivo [17-18]. There is no influx of leukocytes from circulating blood therefore inflammatory complications which affect the interpretation of in vivo assays are minimised. For the purpose of this study, rat aortic ring cultures were used to investigate firstly the anti-vascular and then the anti-angiogenic activity of test compounds.

The successful growth of the aortic rings into a mircovessel network was dependent on several factors including (i) the amount of periaortic fibroadipose tissue left on the ring, (ii) the orientation of the ring, the way which the ring was embedded into the matrigel and (iii) the volume of the matrigel used.

### Enzyme-Based Assay

In order to determine the effect of the bestatin component of the dual acting hybrids, APN enzyme inhibition was determined using a colorimetric method established by Melzig et al. [19]. L-leucine-p-nitroanalide was used as a substrate for leucine aminopeptidase, microsomal from porcine kidney (EC 3.4.11.2). The conversion of l-leucine-p-nitroanalide by the APN enzyme produces a yellow product called p-nitroaniline. The release of p-nitroaniline can be measured spectrophotometrically at 405 nm. The level of the APN enzyme present corresponds directly to the intensity of the colour absorption of the reaction. This assay is widely used to investigate the APN inhibitory properties of a variety of novel compounds [20-23].

### APN Cell-Based Assay

To further investigate the APN inhibitory effect of the hybrids the APN cell-based assay was carried out as previously described [24-26] using both HUVECs and PC-3 cells. In order to exclude any possible cytotoxic effects of the tubulin component of the hybrids that could interfere with the outcome of the results, it was necessary to perform cytotoxicity assays. For this purpose, the MTT assay was used as previously described; however the duration of compounds' incubation was modified to 2 h in order to correlate with the incubation period of APN cell-based assay. The presence of APN-expres sing cells replaced the step of the addition of the actual APN enzyme and the rest of the protocol remained the same. The release of the resulting product p-nitroaniline was measured spectrophotometrically at 405 nm and the level of APN present inside the cells was directly related to the intensity of the colour absorbance.

### APN Expression

To further investigate the effect of compounds on the inhibition of APN expression on both cell lines, an immunofluorescence staining protocol was performed followed by flow cytometry analysis. This method is widely used for the identification and quantification of the level of cell surface expression markers including APN [24, 27-27]. APN is anchored to the plasma membrane but is also present in the cytosol. In the study the level of cytosolic APN was not included in the quantification since the APN antigen was detected using non-permeabilized cells [28].

### Cell Cycle Analysis—Apoptosis

The effect of compounds on cell death was examined using DNA staining followed by flow cytometry analysis. The ability of bestatin to induce apoptosis has been established in a variety of cancer cells [29-30] as well as leukemic cell lines [31-32]. Tubulin targeting agents such as CA-4P demonstrated induction of mitotic cell death after arrest of endothelial cells in mitosis [33-34]. In addition, paclitaxel-induced mitotic block triggered rapid onset of a p53-independent apoptotic pathway [35-37].

Cell cycle analysis was performed as previously described to detect apoptotic DNA fragmentation. During apoptosis DNA is degraded by cellular endonucleases and therefore the apoptotic cells contain less DNA than healthy cells. This results in a sub-G₀/G₁ peak in the fluorescence histogram that can be quantified as the amount of apoptotic cells in a sample. This method was first described by Nicoletti et al. [38] and then optimised by Riccardi et al. [39] and its widely used as a way of examining apoptosis after treatment with anti-cancer compounds [40-42].

### Materials and Methods

### Materials

2-N-morpholinoethanesulphonic acid (MES), ethyleneglycol-bis-β-aminoethylether-N—N-tetra-acetic acid (EGTA), MgCl₂.6H₂O, guanosine triphosphate (GTP, Li salt), glycerol, and podophyllotoxin were obtained from Sigma Aldrich. Coyle Meats Ltd., Garden Lane, Dublin 8 generously donated porcine heads. Dimethyl sulfoxide (DMSO), (3-(4,5-Dimethylthiazol-2-yl)-2,5-diphenyltetrazolium bromide (MTT), fetal bovine serum (FBS), streptomycin/penicillin solution, 0.25% trypsin/EDTA solution, combretastatin A-4 (CA-4), bestatin hydrochloride, L-leucine-p-nitroanilide, HEPES, NaCl, leucine aminopeptidase microsomal from porcine kidney (EC 3.4.11.2), ribonuclease A from bovine pancreas (RNase), propidium iodide (PI), monoclonal mouse anti-α-tubulin antibody, crystal violet Triton-X 100, albumin from bovine serum (BSA) were all purchased from Sigma, Ireland. Anti-human CD13 PE was purchased from ebioscience, UK. Purified anti-mouse CD31 (PECAM-1) antibody was purchased by BD Biosciences, Ireland. Phosphate buffered saline (PBS) tablets, Alexa Fluor® 488 goat anti-mouse IgG, ProLong® gold antifade reagent with DAPI were purchased from Invitrogen, Ireland. EBM-2 medium was purchased from Lonza, Ireland and basement membrane matrix from BD Biosciences, Ireland. Greiner CELLSTAR® 75 cm² cell culture flasks, Greiner 96-well PS cell culture microplates, with solid U-bottom, Greiner 6-well plates and Greiner 12-well plates were purchased from Cruinn, Ireland. Frosted end microscopy slides and cover slips were purchased by VWR, Ireland. Male Wistar rats (200-300 g) were sourced from the BioResources Unit (BRU), Trinity College Dublin. Animals were housed under controlled conditions at 22° C. with 12 hour light and 12 hour dark cycles. All animals had access to food and water ad libitum. Liquid nitrogen was provided by the Department of Chemistry, TCD. All the materials used for the paraffin-embedding of tumour samples and their staining with H&E were kindly provided by Peter Stafford, Department of Zoology, TCD.

### Test Compounds

For the in vitro evaluation, all test compounds were dissolved in DMSO and then diluted in the appropriate medium or buffer to provide the required concentrations. The maximum final DMSO concentration was not >0.1%. Control solutions consisted of 0.1% DMSO and the appropriate medium or buffer. In each experiment a positive control was used. Fresh solutions were prepared prior to use.

### Tubulin Extraction and Purification

A Beckman Ultracentrifuge L8-60M equipped with a Ti 50.5 rotor was used for the ultracentrifugation steps. The procedure used is modified from Shelanski et al (Shelanski, Gaskin et al. 1973). The buffer was prepared with deionised water and the pH of the solution was adjusted to 6.6 by the drop-wise addition of 10M aq. NaOH. The buffer was stored at 4° C. prior to use.

### Tubulin Assembly-Disassembly Assay

Equipment: A Fluostar Optima 96 well plate reader equipped with a thermostat capability.

### Tubulin Assay

The tubulin assembly was followed by observing an increase in optical density of a tubulin solution at 350 nm at 37° C. Typically 1 ml of tubulin solution was diluted with EB (5 ml) and GTP solution (25 μl, 5 μl per ml of EB added) to the mixture. The mixture was vortexed for ten seconds in order to ensure the homogeneity of the mixture. 100 μl of the resultant solution was pipetted into a well in the 96 well plate. The increase in optical density of the tubulin solution was measured over a time course of ten minutes at 350 nm and 37° C. A difference in optical density of 0.1 to 0.15 indicated a tubulin concentration in the range of 2 to 3 mg/ml respectively. Protein concentrations higher or lower than this affected the sensitivity of the assay. Accordingly, adjustments were made to the quantity of buffer added in order to obtain a protein concentration within the desired range. Once the correct concentration was obtained, DMSO (1 μl) was pipetted into three wells in the 96 well plate. Tubulin solution (1000 was subsequently pipetted into each of the same three wells. The plate was immediately incubated at 37° C. and was subjected to orbital shaking for five seconds. The UV absorption was measured at 350 nm over a time course of ten minutes. The maximum slope of the resulting curve was measured. The slope of the blank represents the maximum rate of tubulin polymerisation.

### Cell Lines

Human umbilical vein endothelial cells (HUVECs) were obtained from TCS Cellworks, UK and cultured in endothelial cell basal medium (EBM) supplemented with growth factors and antibiotics (TCS Cellworks, UK). PC-3 human prostate adenocarcinoma cell line was purchased from LGC Standards, UK and cultured in F12K medium supplemented with 0.02 mg/ml sodium pyruvate, 10% FBS and 10 ml/L penicillin/streptomycin solution. PC-3 cell line was selected due to its high expression of APN. Cell and tissue cultures were kept at 37° C. in a humidified atmosphere containing 5% CO₂ at all times.

### Cell Maintenance and Sub-culture

Cell culture experiments were performed under a NuAire class II biological safety cabinet and ensured that aseptic techniques were followed at all times to prevent any contamination. HUVECs/PC-3 cells were culture in 75 cm² cell culture flasks using 15 ml of the appropriate complete medium. Cells were monitored daily using an Olympus C10(41 inverted microscope. Once the cells reached 70-80% confluence they were sub-cultured as following: the old medium was removed from the flasks, the cell layer was gently rinsed with 10 ml PBS, 3 ml of trypsin/EDTA solution was added in each flask and incubated at 37° C. until 90% of the cells rounded up (not longer than 5-10 minutes). The flasks were tapped to detach the cells and 5 ml of complete medium was added into the flasks and then transferred into a centrifuge tube. The flasks were rinsed with a further 5 ml complete medium to collect residual cells and added to the rest of the cells in the tube. Centrifugation took place for 5 minutes at 1000 rpm at 25° C. Following that, the supernatant was removed and 3 ml of fresh medium was added to gently mix the cells.

In order to reseed the cells, 15 ml of complete medium was added in each flask and pre-equilibrated in the CO₂ incubator for at least 30 minutes. The cell suspension was then sub-cultured in the new flasks at 1:4 or 1:6 seeding densities and returned to the incubator. The next day the cells were examined microscopically to ensure the reseeding was successful and pre-equilibrated fresh medium was replaced. Every 2-3 days the medium was changed.

### Cell Count

The estimation of cell densities was determined using a haemocytometer. Once the cells reached 70-80% confluence the same procedure was followed as described above. After the centrifugation step, the cell pellet was resuspended in 10 ml of complete medium and mixed gently. The haemocytometer was cleaned with 70% ethanol and the cover slip was affixed using gentle pressure and circular motions. If the phenomenon of Newton's rings was observed the coverslip was correctly placed on the haemocytometer. The haemocytometer was filled with cell suspension by resting the end of the pipette tip at the edge of the chambers, without overfilling them. The samples were allowed to be drawn out of the pipette by capillary action. Using a x4 objective of the microscope to focus on the grid lines of the haemocytometer the cells were counted in each of the 16 corner squares using a hand tally counter. Cells were included into the count only if they were within the square and any positioned on the left hand or bottom boundary line. Counting was carried until all 4 sets of 16 corner squares were counted and an average count was obtained using this formula (C₁+C₂+C₃+C₄)/4. The haemocytometer is designed so that the number of cells in on set of 16 corner squares is equivalent to the number of cells×10⁴/ml. Therefore, to calculate the number of cells/ml, the average count of cells was multiply by 10 (depending on the total volume of cell suspension) and then by 10⁴. In order to determine the required cell density in each experiment, the following formula was used:

C₁V₁=C₂V₂ were C=concentration and V=volume.

### Cell Proliferation Assay (MTT)

Cell growth inhibition was assessed using the MTT assay. 5×10⁴ cells/ml HUVECs or PC-3 cells (180 μl) were inoculated into each well of a 96-well plate and incubated overnight at 37° C. Compounds or control solutions (20 μl) were added into each well and the plates were incubated for 72 h. CA-4 was used as a positive control. After the incubation period, culture medium was removed and 200 μl of 0.5 mg/ml MTT solution in complete medium was added to each well. Following further 4 h incubation, the solution was removed from each well and 180 μl of DMSO was added to solubilise the formazan crystals resulting from the MTT conversion. Absorbance values for the resulting solutions were read at 584 nm on a microplate reader and cell survival was calculated as the absorbance of treated cells divided by the control. Each compound concentration was tested in quadruplicate and each experiment was performed three independent times. The percentage growth inhibition was determined using the formula below and the results were expressed in terms of IC₅₀ values.

% growth inhibition=100−[(mean Abs of test compound)/(mean Abs of control)]*100

Cell Cycle Analysis—G₂/M phase

Cell cycle arrest at the G₂/M phase was examined by PI-staining. 1×10⁶ cells/ml (1 ml) HUVECs or PC-3 cells were seeded in 6-well plates and incubated overnight at 37° C. Test compounds were diluted in complete medium. CA-4 was used as a positive control. Cells were treated with compounds (1 ml) for 24-h. Next, the cells were harvested by trypsinization and then washed with 3 ml of PBS by centrifugation at 1000 rpm at 25° C. for 5 minutes. Cell pellet was re-suspended first in 300 μl of PBS and then in 3 ml of 70% ethanol (in PBS). Cell suspension was kept at 4° C. until ready for analysis. Prior to analysis, cell suspension was centrifuged at 2000 rpm at 4° C. for 5 minutes and ensured that all the ethanol was removed from each tube. 10 mg/ml RNAse solution and 1 mg/ml PI solution were prepared in PBS. Cell pellet was re-suspended in 300 μl of PBS, whereas 25 μl of RNAse solution and 75 μl of PI solution were added in each sample and incubated at 37° C. for 30 minutes. Samples were analyzed using a Beckman Coulter (Dako) 2-laser CyAn ADP analyser provided by the Department of Biochemistry, TCD. Cell cycle analysis was obtained by examining the PI-stained DNA content, in particular focusing on the G₂/M phase. Each compound concentration was tested in duplicate and each experiment was performed three independent times. The results were expressed in terms of mean percentage of cells found to be present in the G₂/M phase.

### Immunofluorescence Staining of the Microtubule

The effect of the compounds on the microtubule disruption was examined by staining for α-tubulin. Sterile cover slips were placed in each well of a 6-well plate and 1×10⁶ cells/ml (1 ml) HUVECs were seeded and allowed to attach on the cover slips overnight at 37° C. The following day the medium was removed from the wells, replaced by 1 μM of test compounds diluted in medium and incubated for 30 minutes at 37° C. Next, the medium was removed and the cells were fixed in pre-cooled methanol (1 ml) at −20° C. for 30 minutes. Then, the cells were washed twice with 1 ml PBS (5 minutes each time). The primary monoclonal antibody for a-tubulin was diluted in PBS (1:200) and added (1 ml) in each well for 1 h at room temperature (RT). After that, the cells were washed three times with 1 ml PBS and incubated with 1 ml of secondary Alexa Fluor 488 antibody diluted in PBS (1:1000) for 30 minutes at RT in the dark. The cells were washed for a further three times with 1 ml PBS and allowed to air-dry for 10 minutes. The cover slips containing the attached cells were mounted on microscopy slides using ProLong® gold antifade reagent with DAPI (for nuclear staining) and stored at 4° C. until time for analysis. Images were taken using an Olympus FV1000 Point Scanning Confocal Microscope provided by the Department of Biochemistry, TCD. Experiments were performed in duplicate at two independent times. The results were presented as images of the microtubule disruption of endothelial cells.

### Analysis of Endothelial Cell Morphology

The effect of test compounds on endothelial cells' morphology was examined. 1×10⁶ cells/ml HUVECs (1 ml) were seeded into 6-well plates and incubated overnight at 37° C. The next day, the medium was removed and replaced by control solutions (0.1% DMSO in medium) and test compounds diluted in fresh medium. The cells were incubated for 40 minutes at 37° C. Following that, each well was washed with medium to remove detached cells and incubated with fresh medium for a further 1 h or 3 h at 37° C. Next, the medium was removed and 0.5% crystal violet solution was prepared in 20% ethanol and 1 ml was added in each well for 15 minutes at RT. The wells were then washed 3 times with d. water (5 minutes each time). Wells were allowed to air dry and cell morphology was examined under an Olympus CKX41 inverted microscope using a x4 objective. Images were captured with an E-330 SLR camera system 1 h and 3 h after the washout of compounds. Digital images were processed using Cell̂ A software (Mason Technology, Ireland). The experiments were performed in triplicate and the results were presented as images of the morphological changes of endothelial cells after 1 h and 3 h of drug washout.

### In Vitro Angiogenesis Assay

In order to analyse the effect of test compounds in a co-culture of human endothelial cells and fibroblasts, the AngioKit assay was used provided by TCS Cellworks, UK. The assay was a co-culture in 24-well plate format of HUVECs and matrix-producing cells at the earliest stages of tubule formation. Culture was maintained in endothelial growth medium (EGM) at 37° C. with 5% CO₂ humidified atmosphere. Controls and test compounds diluted in medium were added on day 1. Culture medium was changed every 2-3 days until day 12. CD31 staining kit was used in order to visualise the tubule formation. Mouse anti-human CD31 antibody was diluted in blocking buffer, added into each well and incubated for 1 h at 37° C. Then the wells were washed with the blocking buffer for 3 times (5 minutes each time). The secondary antibody (goat anti-mouse IgG alkaline phosphatise conjugate) was also diluted in blocking buffer, added in each well and the plate was incubated for a further 1 h at 37° C. After that, the wells were washed three times with d. water (5 minutes each time). The insoluble substrate was dissolved in d. water and added in each well. The plate was incubated at 37° C. for 10-15 minutes until the tubules developed a dark purple colour. Finally, the wells were washed three times with d. water (5 minutes each time) and left to air dry. Images were taken using an Olympus CKX41 inverted microscope and images were captured with an E-330 SLR camera system and processed using Cell̂ A software (Mason Technology, Ireland). The experiments were performed in duplicate. The tubule mean pixel density area in each image was estimated using Adobe Photoshop CS4 and results were presented as the mean pixel density area (pixels²) as well as images of the tubule formation.

### Aortic Ring Assay (Anti-Vascular Model)

The aortic ring assay is an ex vivo assay which allows the analyst to examine the inhibition/stimulation of angiogenesis as well as the breakdown of existing vasculature. Basement membrane matrix (matrigel) was stored at −20° C. Prior to use, matrigel vial was placed on ice and kept at 4° C. overnight in order to thaw. The matrigel was kept on ice at all times for the duration of the experiment. Aortic rings were obtained by excising the thoracic aorta of 5-10 weeks old Wistar male rats and removing the fibroadipose tissue from the aortic wall. The rings were obtained by cross-sectioning the aorta at ˜1 mm intervals, removed any blood clots by washing several times with EBM-2. Matrigel (80 μl) was placed uniformly in each well of a 12-well plate and the aortic rings were then placed individually in between the matrigel layer with the lumen perpendicular to the surface of the plate. The matrigel solution was allowed to solidify for 30-40 minutes at 37° C. and 1 ml EBM-2 medium (supplemented with 2% FBS and 1 mM streptomycin/penicillin solution) was added to each culture. The aortic rings were kept in a humidified CO₂ incubator at 37° C. and fresh medium was replaced three times a week starting from day 3. Once an established microvessel network was developed (day 7-9), test compounds diluted in EBM-2 medium (1 ml) were added to each well and incubated at 37° C. for 24 h. The cultures were monitored for microvessel breakdown over time and images were taken 1 h, 4 h and 24 h after treatment using an Olympus CKX41 inverted microscope. Images were captured with an E-330 SLR camera system and processed using Cell̂ A software. Each compound concentration was tested in duplicate. Results were presented as images showing the microvessel breakdown at each time point.

### APN Assay Buffer

For the APN enzyme and APN cell-based assay HEPES buffer (50 mM HEPES, 154 mM NaCl) was prepared weekly and kept at 4° C. The pH was adjusted to 7.3-7.4 prior to use.

### APN Expression of HUVECs and PC-3 Cells

In order to quantify the APN expression level on the surface of HUVECs and PC-3 cells an immunofluorescence flow cytometry protocol was followed. Cells were harvested by trypsinization and washed in ice-cold PBS by centrifugation at 1000 rpm at 25° C. for 5 minutes. A CD13 PE conjugated antibody (1:10) was added to 1×10⁶ cells/ml and kept on ice for 15 minutes in dark. The cells were then washed with 3 ml of PBS by centrifugation at 1000 rpm at 25° C. for 5 minutes and immediately analyzed by a Beckman Coulter (Dako) 2-laser CyAn ADP analyser provided by the Department of Biochemistry, TCD. APN/CD13 levels were estimated as median fluorescence intensity. The experiments were performed in triplicate at three independent times.

### APN Enzyme-Based Assay

APN enzyme activity was examined spectrophotometrically using L-leucine-p-nitroanilide as an APN substrate. Briefly, 50 μA of L-leucine-p-nitroanilide was added in each well of a 96-well plate to give a final concentration of 2 mM. Test compounds were diluted in HEPES buffer to give a range of concentrations and 100 μl were added into each well. Bestatin hydrochloride was used as a positive control. The reaction was initiated by adding 10 μl of leucine aminopeptidase microsomal to give a final concentration of 5 mUnits and the plate was incubated for 1 h at 37° C. The absorbance was measure at 405 nm using a FLUOstar OPTIMA microplate reader (BMG labtech). Each compound concentration was tested in quadruplicate and each experiment was performed three independent times. The percentage APN inhibition was determined using the formula below and where appropriate IC₅₀ values were obtained.

% APN inhibition=100−[(mean Abs of test compound)/(mean Abs of control)]*100

### 4.2.8 APN Cell-Based Assay

HUVECs/PC-3 cells' surface APN activity was estimated spectrophotometrically using L-leucine-p-nitroanilide as an APN substrate. 1×10⁶ cells/ml (150 μA) were seeded in 96-well plate and incubated overnight at 37° C. The following day cell medium was replaced by various concentrations of compounds diluted in HEPES buffer (100 μl). Bestatin hydrochloride was dissolved in HEPES buffer and used as a positive control. L-leucine-p-nitroanilide (50 μl) was added to each well to give a final concentration of 2 mM. Following 2 h incubation at 37° C., absorbance was measured at 405 nm using a FLUOstar OPTIMA microplate reader (BMG labtech). Each compound concentration was tested in quadruplicate and each experiment was performed at three independent times. The percentage APN inhibition was determined using the formula below and where appropriate, IC₅₀ values were obtained.

% APN inhibition=100−[(mean Abs of test compound)/(mean Abs of control)]*100

### 4.2.9 Inhibition of APN Expression

APN/CD13 expression of HUVECs and PC-3 cells was evaluated followed treatment with 30. 1×10⁶ cells/ml (1 ml) were seeded in 6-well plates and incubated overnight at 37° C. The following day cells were treated with test compounds diluted in complete medium for 2 h or 24 h. Bestatin hydrochloride was used as a positive control. Cells were harvested by trypsinization and washed in ice-cold PBS by centrifugation at 1000 rpm at 25° C. for 5 minutes. A CD13 PE conjugated antibody was added to the cells (10 μl/100 μl) and kept on ice for 15 minutes. The cells were then washed with 3 ml of PBS by centrifugation at 1000 rpm at 25° C. for 5 minutes and immediately analyzed by a Beckman Coulter (Dako) 2-laser CyAn ADP analyser provided by the Department of Biochemistry, TCD. APN/CD13 levels were estimated as median fluorescence intensity. Each compound concentration was tested in duplicate and each experiment was performed three independent times. The results were expressed as a percentage of the mean CD13 PE median fluorescence intensity.

### Statistical Analysis

Statistical analysis of the data was carried out using GraphPad Prism, version 5. Percentage inhibition values of each compound at each concentration are expressed as mean±SEM (standard error of mean). Where appropriate, IC₅₀ values were generated. In order to determine whether there was a significant difference between control and treated groups the Student's t-test was applied using GraphPad Prism.

### Results

### Tubulin Binding Data for Compound 1.

The tubulin binding data for Compound 1 was compared that of the comparator compound which was not functionalised at the Z-position ie CH₂ vs Carbonyl. The IC50 obtained for 1 was 1.05 μM R² 0.923 vs 6.7 μM R² 0.923. When compared to combretastatin A-4 a value of 1.45 μM R² 0.999 was obtained. The isomer of compound 1, namely 12 was inactive under the assay conditions used.

### Compounds 1 and 28

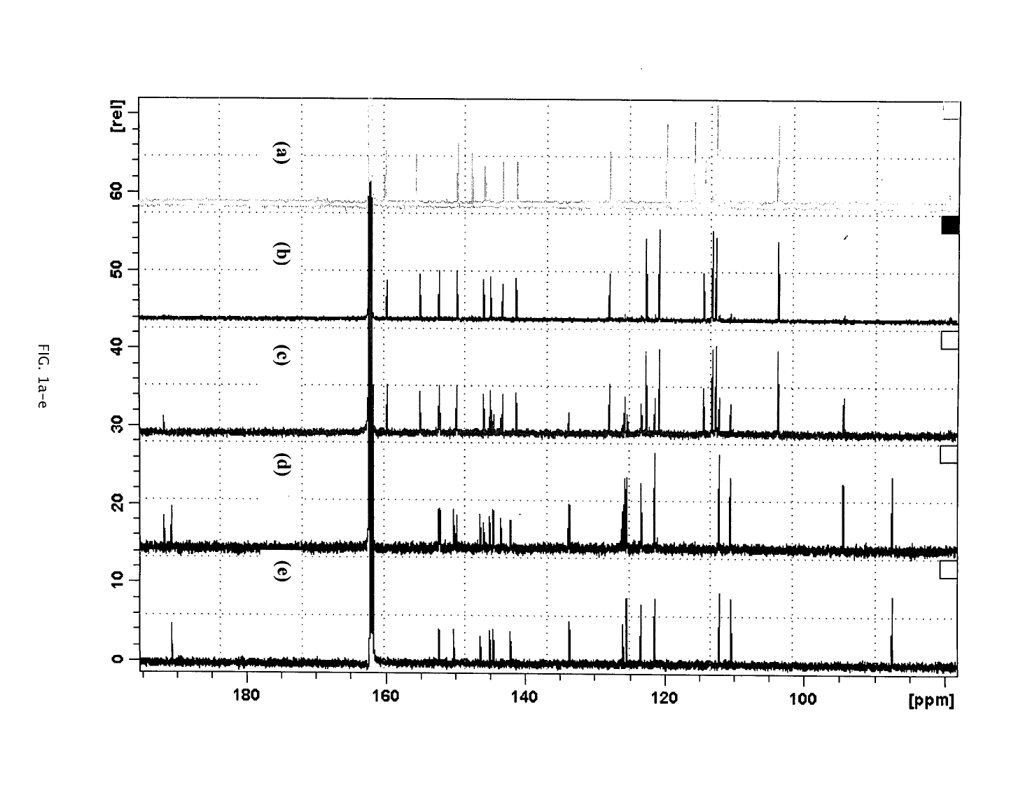

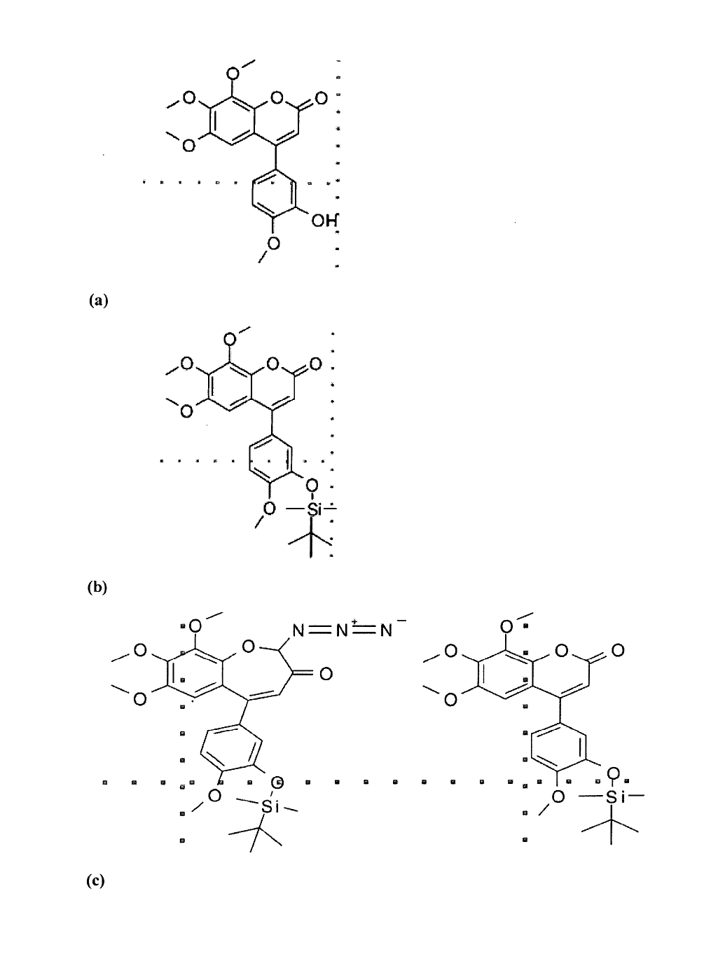

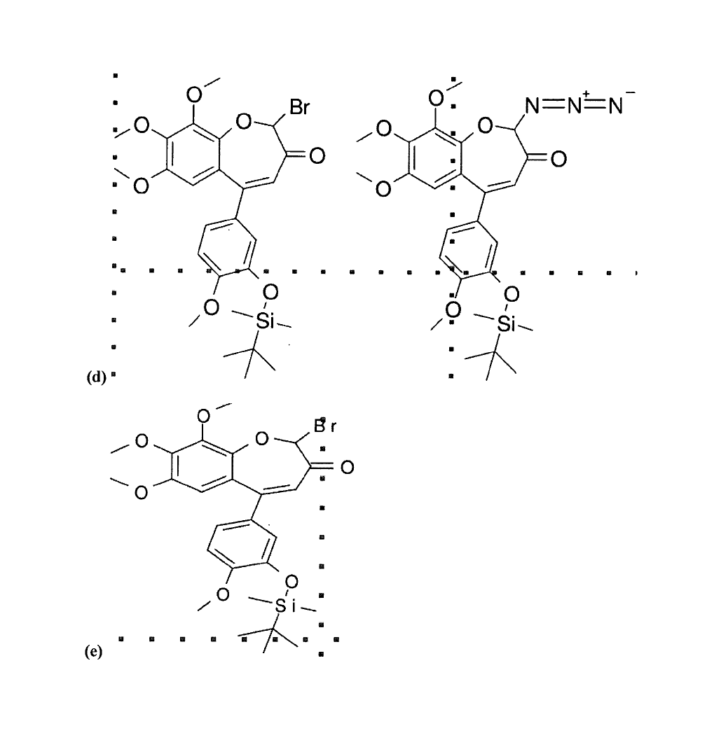

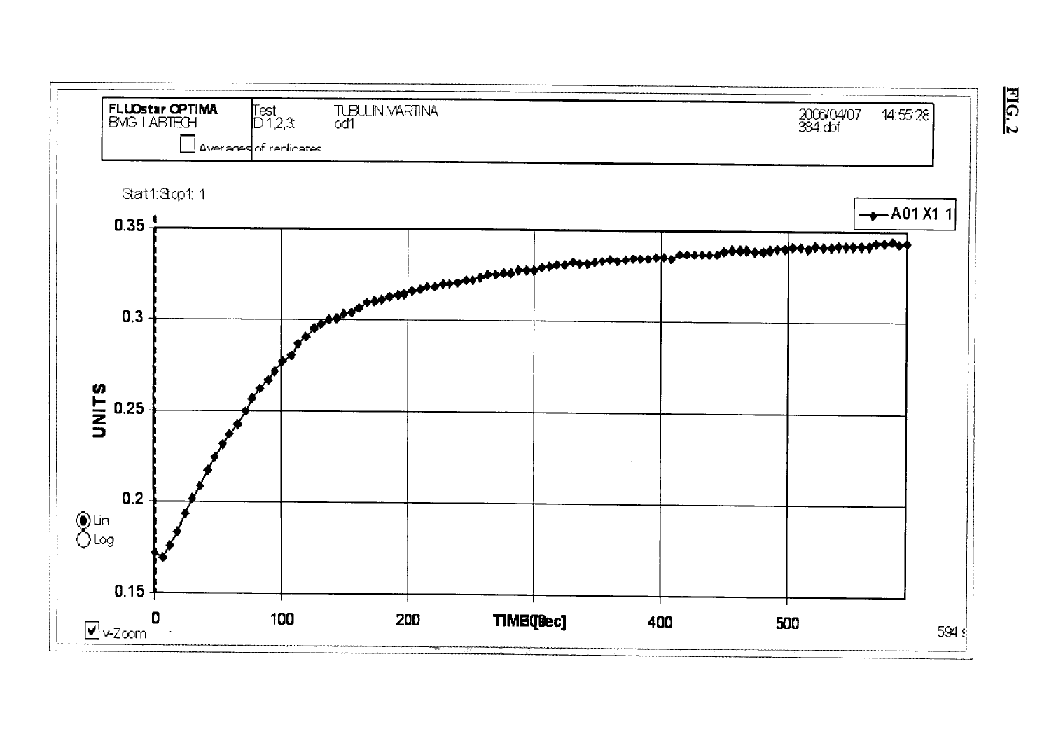

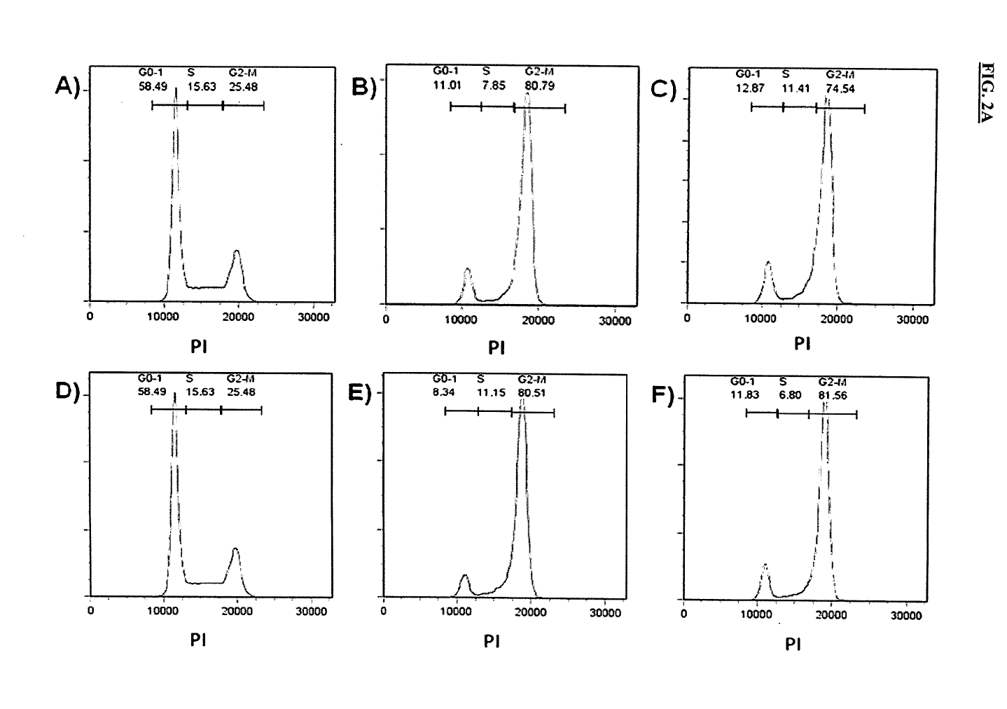

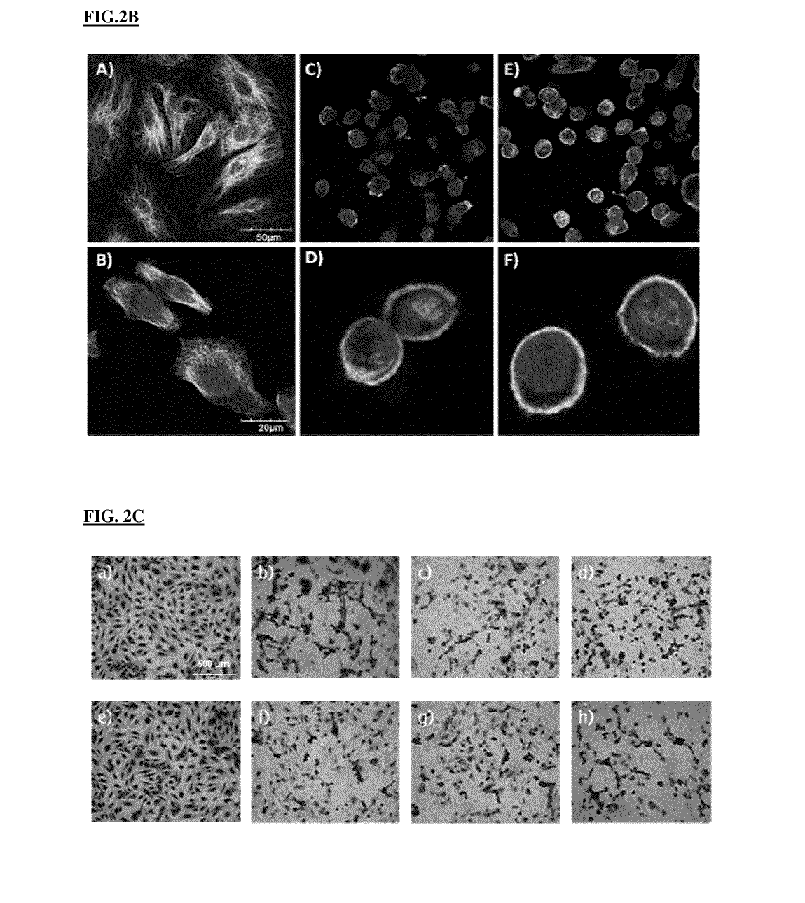

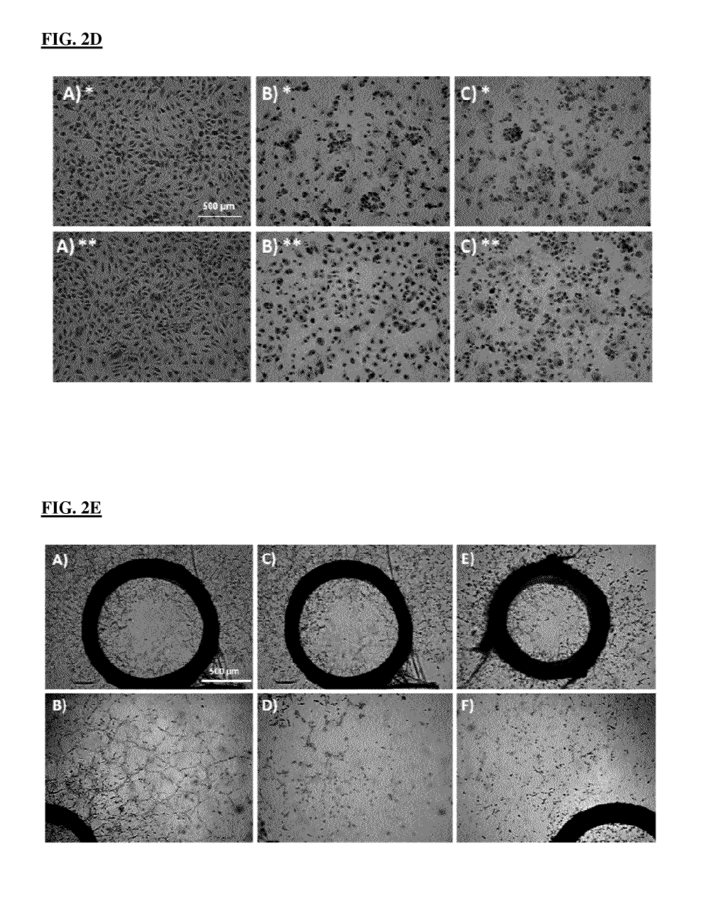

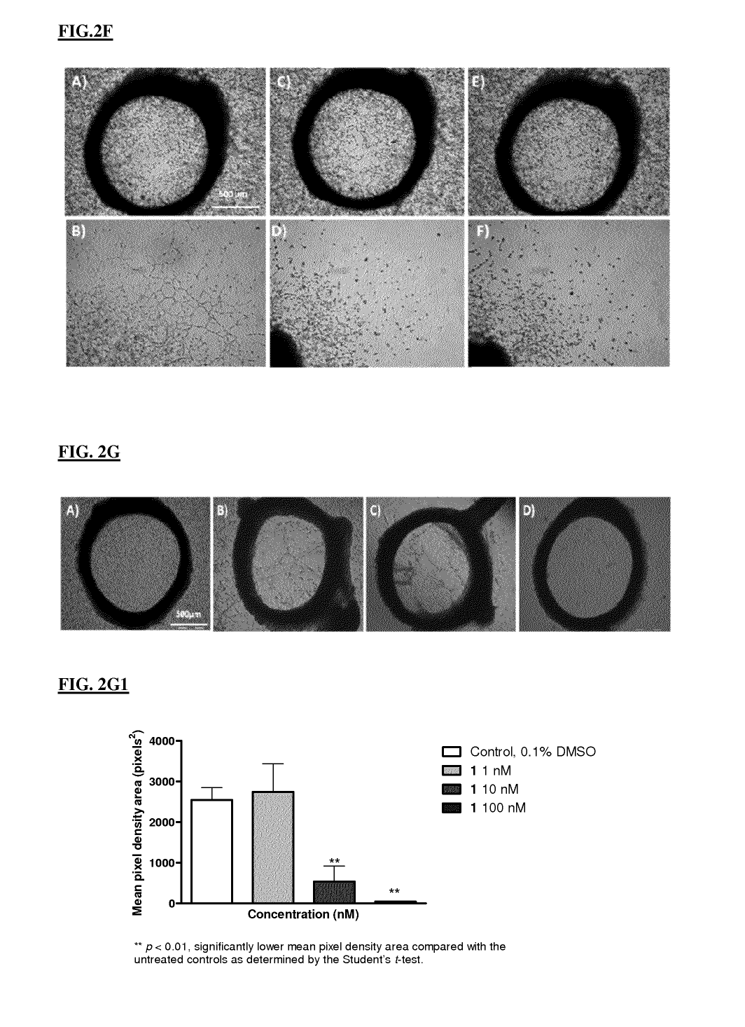

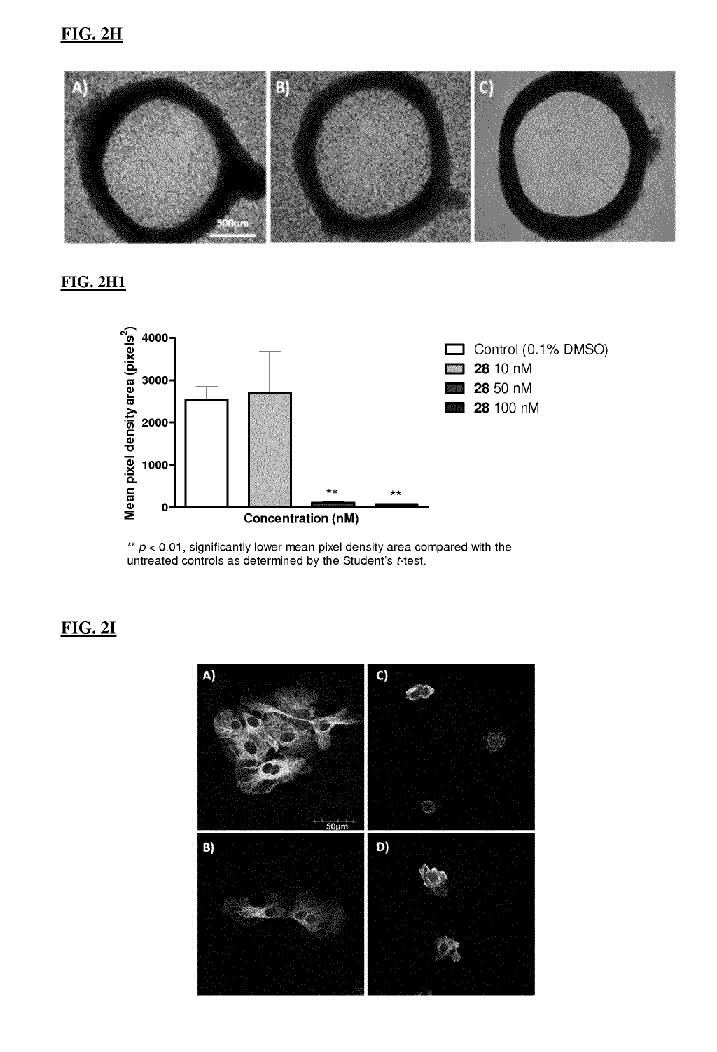

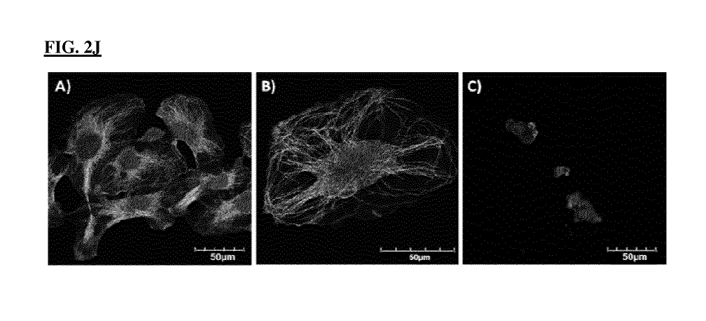

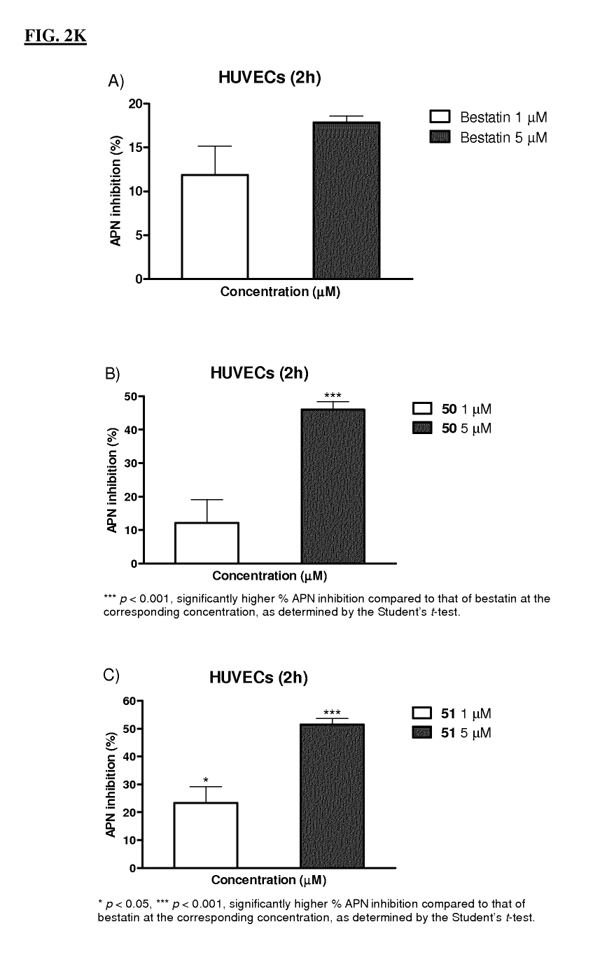

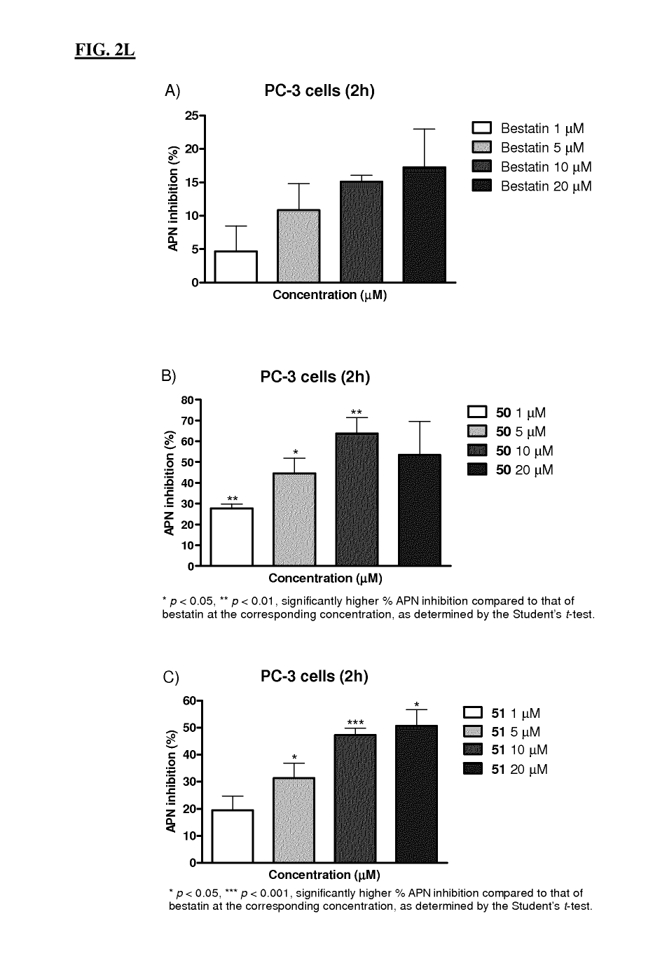

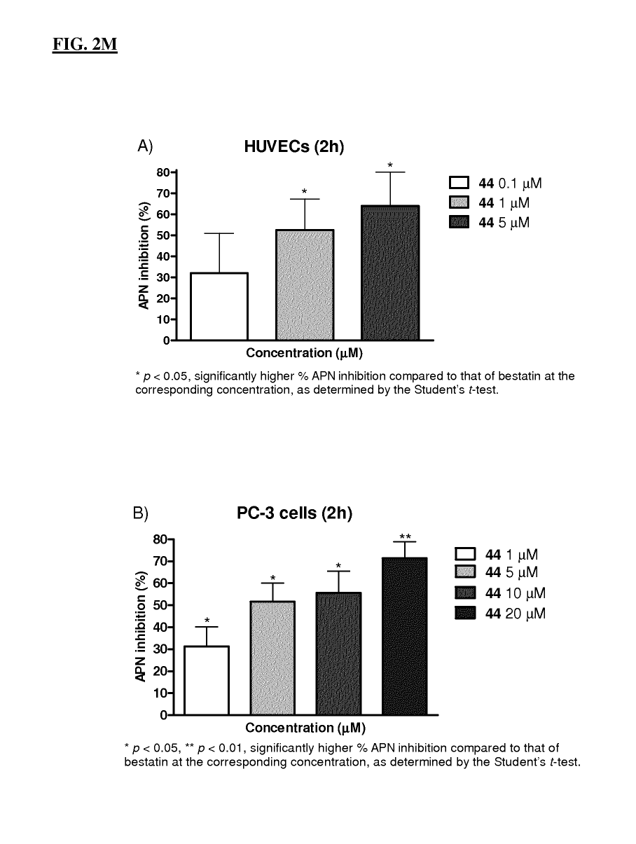

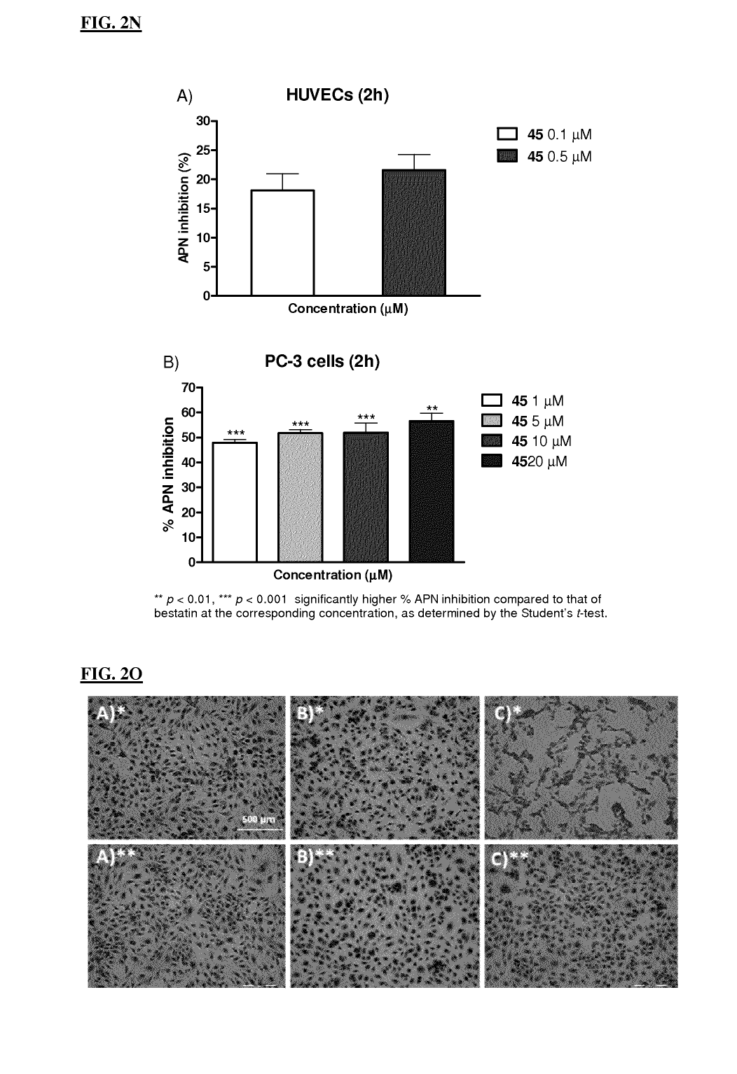

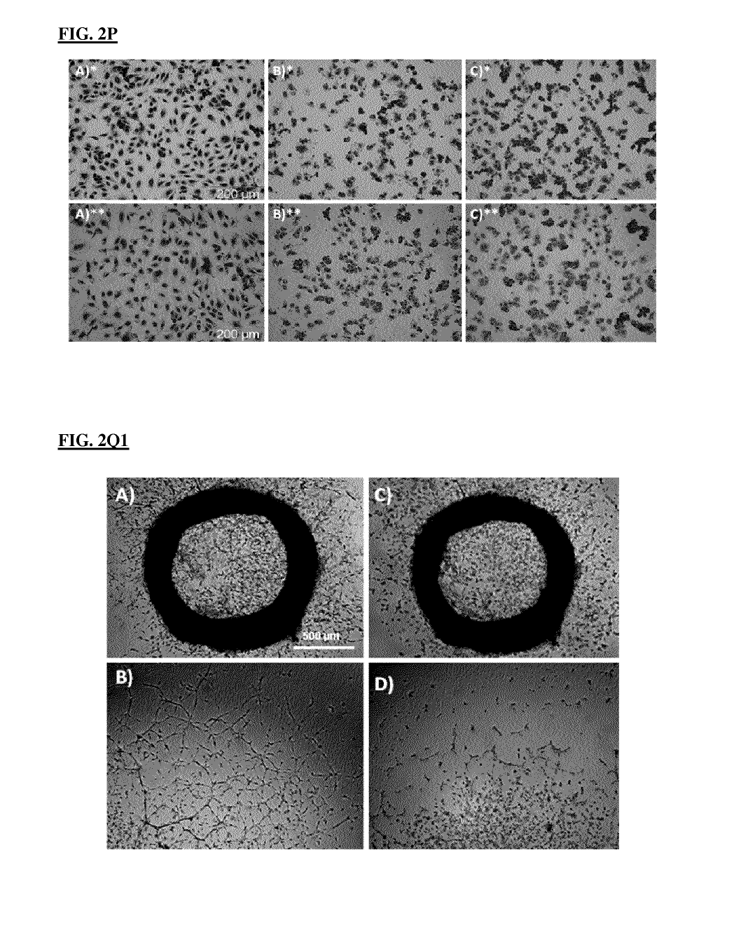

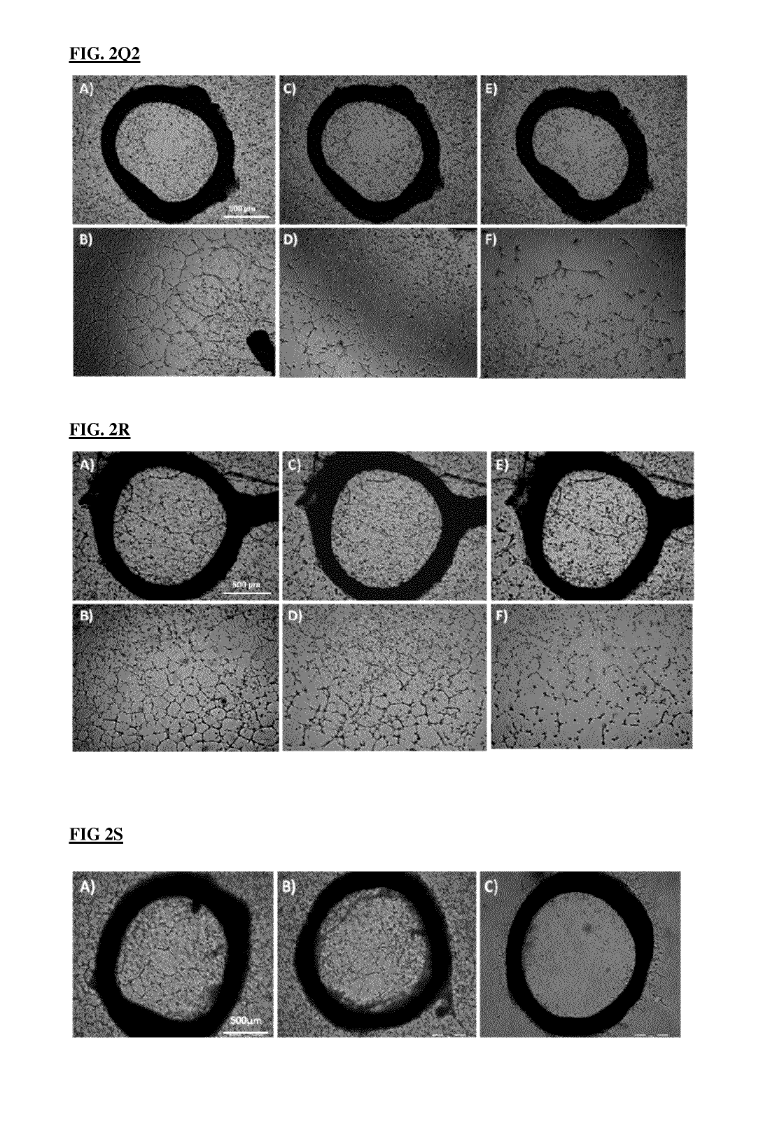

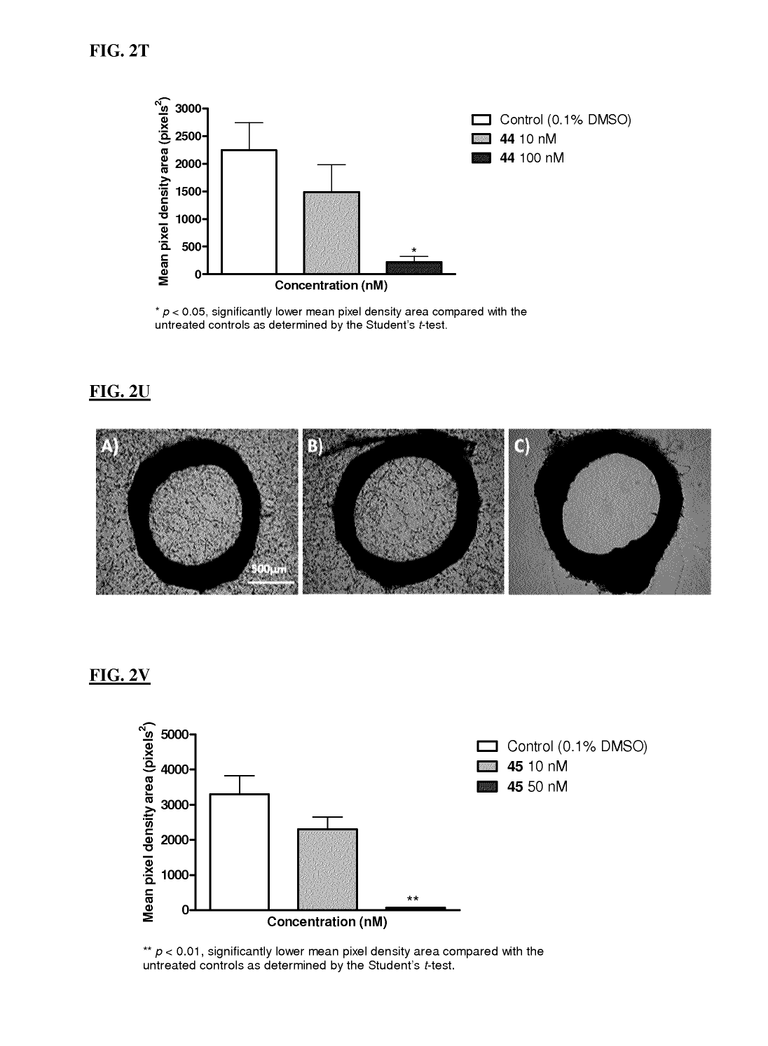

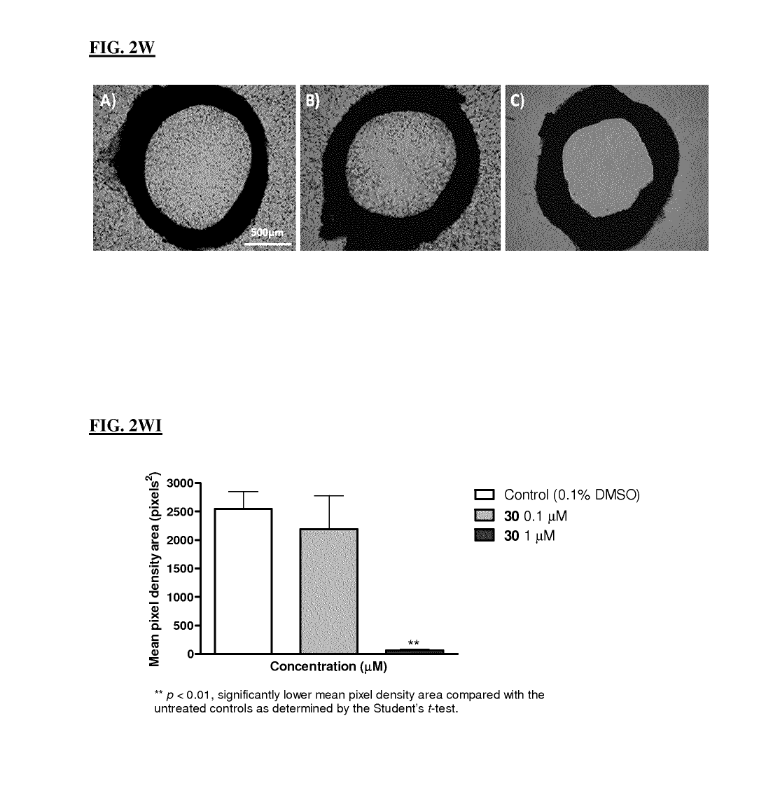

Cell cycle analysis was performed on HUVECs after 24 h treatment with 1 and 28. Treated cells were fixed, stained with PI and processed through flow cytometry. 1 exhibited a significant (p<0.01) effect on the cell cycle inducing an arrest at the G₂/M phase (at 0.5 μM 86.3±2.3% and at 1 μM 79.35±1.7%). Similarly, 0.5 μM and 1 μM of 28 demonstrated a significant (p<0.01) increase in the percentage of cells present in the G₂/M phase; 81.25±0.6% and 76.07±4.82% respectively, as shown in FIG. 2a.

### Compounds 1 and 28

The effect of 1 μM 1 and 28 on microtubule organization was determined. HUVECs were incubated with test compounds for 30 minutes and stained for α-tubulin (green) and nucleus (blue). Images were taken using a confocal microscope. Microtubule disruption was observed immediate after treatment with 1 and 28 and the level of disruption was the same as with both compounds (FIG. 2b). Microtubule fibres were absent in the treated cells.

### Effect on Morphology Compounds 1 and 28

The effect 1 and 28 on the endothelial cell morphology was evaluated. Both compounds demonstrated their activity at 0.1 μM by modifying the cells' structure and exhibited similar profile as CA-4 (FIGS. 2c and 2d). The reversibility of this effect was observed following 3 h of drug washout were the cells regained their shape (FIG. 2c).

### Anti-Vascular Effects of Compounds 1 and 28.

Compounds 1 and 28 were examined for their anti-vascular effect on existing microvessel network. Aortic rings were treated with 100 nM of either 1 or 28 and showed evident changes in the microvessels' structure after 4 h of treatment. This effect was time-dependent with the greater disruption after 24 h of treatment as shown in FIGS. 2e and 2f. These compounds showed greater anti-vascular activity over the comparator compounds without functionalisation on the B-ring.

### Compound 1 and 28

The anti-angiogenic effect of 1 and 28 was determined using the aortic ring assay. There was no inhibitory effect observed after treatment with 1 nM 1, however 10 nM and 100 nM significantly (p<0.01) inhibited the formation of microvessels and the response was dose-dependent (FIGS. 2g and 2h. The aortic rings treated with 10 nM 28 did not demonstrate a decrease in microvessel formation, but 50 nM and 100 nM were sufficient to cause complete growth inhibition (FIG. 2g and FIG. 2h).

44 was examined for its ability to interfere with the microtubule formation of HUVECs. Treatment with 1 μM of 44 for 30 minutes was able to induce microtubule disruption by reducing the level of stained α-tubulin as shown in FIG. 2i. Long microtubule fibres were present in the control treated cells, whereas in the 44 treated cells fibres were absent.

### Compound 45

The tubulin cytoskeleton of HUVECs was examined after treatment with 1 μM 45 for 30 minutes and then stained for α-tubulin and nucleus. The disruption of microtubule organisation by 45 was clear, as the amount of stained tubulin was dramatically reduced and there was no presence of long microtubule filaments (FIG. 2j).

The ability of 50 and 51 to inhibit the activity of the APN enzyme present on HUVECs and PC-3 cells was assessed in the APN cell-based assay. Firstly, the activity of test compounds and bestatin were examined using HUVECs. Bestatin's APN inhibitory effect was examined at 1 μM and 5 μM and determined to be 11.88±3.27% and 17.86±0.75 respectively. Both hybrids demonstrated satisfactory APN inhibition against HUVECs at concentrations below 10 μM. 50 exhibited 12.15±7% at 1 μM and 46±2.3% at 5 μM, whereas 51 showed 23.4±5.8% at 1 μM and 51.5±2.12% at 5 μM as shown in FIG. 2k. 50 and 51 revealed significantly (p<0.001) superior APN inhibition to that of bestatin at 5 μM in HUVECs by an almost 3-fold increase in activity.

Next, the effect of hybrids and bestatin on the APN inhibition of PC-3 cells was determined. Bestatin was examined at 1, 5, 10 and 20 μM and the percentage APN inhibition was established to be 10.84±3.24, 15.1±0.97, 17.3±5.72 and 36.11±0.9% respectively. Similarly, the activity of 50 and 51 was evaluated at 1, 5, 10 and 20 μM and the percentage inhibition was determined to be 27.76±2.1, 44.6±7.3, 63.7±7.68 and 53.43±16.14% for 44 and 31.37±5.48, 47.25±2.05, 50.72±6.02 and 61.9±0.99% for 51 respectively (FIG. 21). The inhibitory properties of both hybrids against the APN enzyme present on PC-3 cells were significantly proven to be greater to that of bestatin by more than 2-fold.

### Compound 44

44 was evaluated for its APN inhibitory properties against HUVECs and PC-3 cells at non-cytoxic concentrations. The effect of 44 on HUVECs was analysed at 0.1, 1 and 5 μM where the percentage APN inhibition was determined to be 31.9±19.0, 52.6±14.7 and 64±16.1% respectively (FIG. 2m A). 44 indicated improved activity against APN compared to that of bestatin by approximately 4- to 5-fold increases in activity. The APN inhibition of 44 was also established using PC-3 cells at 1, 5, 10 and 20 μM and estimated to be 31.3±8.9, 51.7±8.5, 55.7±9.8 and 71.4±7.5% respectively (FIG. 2m B). 44 activity was significantly superior to that of bestatin by a 2-fold increase.

### Compound 45

45 was assessed for its APN inhibitory properties in the APN cell-based assay. Due to its potent cytotoxic effect on HUVECs, 45 was only tested at 0.1 and 0.5 μM in the APN assay and the percentages APN inhibition were 18.1±2.9% and 21.6±2.2% respectively (FIG. 2n A). This result suggests that 45 has a superior activity to that of bestatin in the HUVECs-based APN assay. The percentages APN inhibition caused by 1, 5, 10 and 20 μM of 45 were determined using PC-3 cells and found to be 47.91±1.23, 51.71±1.38, 51.87±3.89 and 56.55±3.13, respectively (FIG. 2n B). 45 was significantly more potent APN inhibitor than bestatin in the PC-3 based assay.

### Morphololgy Effects of 44

44 was assessed for its ability to disrupt the endothelial cell morphology. HUVECs were treated with 0.1 and 1 μM 44 and visualised for any difference in their appearance (FIG. 2o). At 0.1 μM 44 a minor effect was observed in the endothelial cell morphology, however at 1 μM 44 cell monolayer was disturbed and cells' appearance was altered. Despite the drastic morphological changes, endothelial cells regain normality after 3 h of drug washout indicating a reversible effect.

The indirect anti-vascular effect of 45 on endothelial cell morphology was determined. HUVECs were incubated with 0.1 and 1 μM 45 and their appearance was examined. A severe effect on cell morphology was noted after treatment with 45 at both concentrations, where the cell monolayer seems disrupted and cells appeared to shrink and round up. Following a 3 h incubation period after drug washout, cells did not seem to regain their original shape, signifying 45 potency.

### Compound 44

44 was evaluated for its ability to interfere with established microvessel network. Incubation of the aortic rings with 0.1 μM did not result in a clear anti-vascular effect, and thus 1 μM was used. An apparent microvessel breakdown was observed at 1 μM 44 after a 24 h incubation period (FIG. 2q1).

### Compound 45

The anti-vascular effect of 45 was investigated in the aortic ring assay. Treatment of the ring cultures with 1 and 10 nM 45 did not demonstrate any obvious anti-vascular activity, whereas 50 nM was sufficient to disrupt the microvessel network even after 4 h of treatment, and the effect was more evident after 24 h (FIG. 2q2).

### Compound 30

30 was examined for any evidence of anti-vascular properties in the aortic ring assay. At 1 μM, 30 demonstrated satisfactory activity after 4 h of treatment and the effect was more evident following a 24 h incubation period, where a clear microvessel disruption was observed (FIG. 2r).

### 5.3.2.2 Compound 44

The anti-angiogenic properties of 44 were examined in the aortic ring assay. At 10 nM of 44 there was a slight decrease in the microvessel density of the aortic rings, although not statistically significant. Treatment with 100 nM of 44 demonstrated significant (p<0.05) reduction in the level of microvessel growth (FIGS. 2s and 2t).

### 5.3.2.3 Compound 45

The ability of 45 to inhibit angiogenesis was evaluated in the aortic ring assay. Microvessel density was insignificantly decreased at 10 nM of 45, whereas 50 nM caused almost complete inhibition (p<.0.01) of the microvessel growth (FIGS. 2u and 2v).

### Compound 30

The anti-angiogenic effect of 30 was determined in the aortic ring assay. Cultured rings treated with 0.1 μM 30 showed a negligible reduction in the microvessel area, whereas treatment with 1 μM 30 resulted in significantly (p<0.01) lower microvessel density compared to the control treated ring cultures.

The invention is not limited to the embodiment hereinbefore described which may be varied in construction and detail without departing from the spirit of the invention.

### REFERENCES

[1] Mosmann, T. Rapid colorimetric assay for cellular growth and survival: application to proliferation and cytotoxicity assays. J Immunol Methods. 1983, 65(1-2), 55-63.
[2] Rabinovitch, P. Introduction to Cell Cycle Analysis. Basics of DNA Cell Cycle Analysis.
[3] Lin, H. L.; Chiou, S. H.; Wu, C. W.; Lin, W. B.; Chen, L. H.; Yang, Y. P.; Tsai, M. L.; Uen, Y. H.; Liou, J. P. Chi, C. W. Combretastatin A4-induced differential cytotoxicity and reduced metastatic ability by inhibition of AKT function in human gastric cancer cells. J Pharmacol Exp Ther. 2007, 323(1), 365-373.
[4] Jung, H. J.; Shim, J. S.; Lee, H. B.; Kim, C. J.; Kuwano, T.; Ono, M. Kwon, H. J. Embellistatin, a microtubule polymerization inhibitor, inhibits angiogenesis both in vitro and in vivo. Biochem Biophys Res Commun. 2007, 353(2), 376-380.
[5] Arora, S.; Wang, X. I.; Keenan, S. M.; Andaya, C.; Zhang, Q.; Peng, Y. Welsh, W. J. Novel microtubule polymerization inhibitor with potent antiproliferative and antitumor activity. Cancer Res. 2009, 69(5), 1910-1915.
[6] Krishan, A. Rapid flow cytofluorometric analysis of mammalian cell cycle by propidium iodide staining. J. Cell Biol. 1975, 66(1), 188-193.
[7] Fulwyler, M. J. Electronic separation of biological cells by volume. Science. 1965, 150(3698), 910-911.
[8] Nihei, Y.; Suzuki, M.; Okano, A.; Tsuji, T.; Akiyama, Y.; Tsuruo, T.; Saito, S.; Hori, K. Sato, Y. Evaluation of antivascular and antimitotic effects of tubulin binding agents in solid tumor therapy. Jpn J Cancer Res. 1999, 90(12), 1387-1395.
[9] Joncour, A.; Liu, J. M.; Decor, A.; Thoret, S.; Wdzieczak-Bakala, J.; Bignon, J. Baudoin, O. Synthesis of anti-microtubule biaryls and preliminary evaluation as vascular-disrupting agents. ChemMedChem. 2008, 3(11), 1731-1739.
[10] Ahmed, B.; Van Eijk, L. I.; Bouma-Ter Steege, J. C.; Van Der Schaft, D. W.; Van

Esch, A. M.; Joosten-Achjanie, S. R.; Lambin, P.; Landuyt, W. Griffioen, A. W. Vascular targeting effect of combretastatin A-4 phosphate dominates the inherent angiogenesis inhibitory activity. Int J Cancer. 2003, 105(1), 20-25.
[11] Giuliani, N.; Colla, S.; Lazzaretti, M.; Sala, R.; Roti, G.; Mancini, C.; Bonomini, S.; Lunghi, P.; Hojden, M.; Genestreti, G.; Svaldi, M.; Coser, P.; Fattori, P. P.; Sammarelli, G.; Gazzola, G. C.; Bataille, R.; Almici, C.; Caramatti, C.; Mangoni, L. Rizzoli, V. Proangiogenic properties of human myeloma cells: production of angiopoietin-1 and its potential relationship to myeloma-induced angiogenesis. Blood. 2003, 102(2), 638-645.
[12] Nicosia, R. F. and Ottinetti, A. Growth of microvessels in serum-free matrix culture of rat aorta. A quantitative assay of angiogenesis in vitro. Lab Invest. 1990, 63(1), 115-122.
[13] Villaschi, S, and Nicosia, R. F. Angiogenic role of endogenous basic fibroblast growth factor released by rat aorta after injury. Am J Pathol. 1993, 143(1), 181-190.
[14] Nicosia, R. F.; Nicosia, S. V. Smith, M. Vascular endothelial growth factor, platelet-derived growth factor, and insulin-like growth factor-1 promote rat aortic angiogenesis in vitro. Am J Pathol. 1994, 145(5), 1023-1029.
[15] Nicosia, R. F.; Lin, Y. J.; Hazelton, D. Qian, X. Endogenous regulation of angiogenesis in the rat aorta model. Role of vascular endothelial growth factor. Am J Pathol. 1997, 151(5), 1379-1386.
[16] Zhu, W. H.; Maclntyre, A. Nicosia, R. F. Regulation of angiogenesis by vascular endothelial growth factor and angiopoietin-1 in the rat aorta model: distinct temporal patterns of intracellular signaling correlate with induction of angiogenic sprouting. Am J Pathol. 2002, 161(3), 823-830.
[17] Nicosia, R. F.; Bonanno, E. Villaschi, S. Large-vessel endothelium switches to a microvascular phenotype during angiogenesis in collagen gel culture of rat aorta. Atherosclerosis. 1992, 95(2-3), 191-199.
[18] Nicosia, R. F.; Tchao, R. Leighton, J. Angiogenesis-dependent tumor spread in reinforced fibrin clot culture. Cancer Res. 1983, 43(5), 2159-2166.
[19] Melzig, M. F. and Bormann, H. Betulinic acid inhibits aminopeptidase N activity. Planta Med. 1998, 64(7), 655-657.
[20] Albrecht, S.; Defoin, A.; Salomon, E.; Tarnus, C.; Wetterholm, A. Haeggstrom, J. Z. Synthesis and structure activity relationships of novel non-peptidic metallo-aminopeptidase inhibitors. Bioorg Med. Chem. 2006, 14(21), 7241-7257.
[21] Flipo, M.; Florent, I.; Grellier, P.; Sergheraert, C. Deprez-Poulain, R. Design, synthesis and antimalarial activity of novel, quinoline-based, zinc metallo-aminopeptidase inhibitors. Bioorg Med Chem. Lett. 2003, 13(16), 2659-2662.
[22] Andersson, L.; Isley, T. C. Wolfenden, R. alpha-aminoaldehydes: transition state analogue inhibitors of leucine aminopeptidase. Biochemistry. 1982, 21(17), 4177-4180.
[23] Lejczak, B.; Kafarski, P. Zygmunt, J. Inhibition of aminopeptidases by aminophosphonates. Biochemistry. 1989, 28(8), 3549-3555.
[24] Terauchi, M.; Kajiyama, H.; Shibata, K.; Ino, K.; Nawa, A.; Mizutani, S. Kikkawa, F.

Inhibition of APN/CD13 leads to suppressed progressive potential in ovarian carcinoma cells. BMC Cancer. 2007, 7, 140.
[25] Cui, S. X.; Qu, X. J.; Gao, Z. H.; Zhang, Y. S.; Zhang, X. F.; Zhao, C. R.; Xu, W. F.; Li, Q. B. Han, J. X. Targeting aminopeptidase N (APN/CD13) with cyclic-imide peptidomimetics derivative CIP-13F inhibits the growth of human ovarian carcinoma cells. Cancer Lett. 2010, 292(2), 153-162.
[26] Gao, J. J.; Gao, Z. H.; Zhao, C. R.; Yuan, Y.; Cui, S. X.; Zhang, X. F.; Cheng, Y. N.; Xu, W. F.; Tang, W. Qu, X. J. LYP, a novel bestatin derivative, inhibits cell growth and suppresses APN/CD13 activity in human ovarian carcinoma cells more potently than bestatin. Invest New Drugs. 2011, 29(4), 574-582.
[27] Bauvois, B.; Van Weyenbergh, J.; Rouillard, D. Wietzerbin, J. TGF-beta 1-stimulated adhesion of human mononuclear phagocytes to fibronectin and laminin is abolished by IFN-gamma: dependence on alpha 5 beta 1 and beta 2 integrins. Exp Cell Res. 1996, 222(1), 209-217.
[28] Jamur, M. C. and Oliver, C. Permeabilization of cell membranes. Methods Mol. Biol. 2010, 588, 63-66.
[29] Ezawa, K.; Minato, K. Dobashi, K. Induction of apoptosis by ubenimex (Bestatin) in human non-small-cell lung cancer cell lines. Biomed Pharmacother. 1996, 50(6-7), 283-289.
[30] Sekine, K.; Fujii, H.; Abe, F. Nishikawa, K. Augmentation of death ligand-induced apoptosis by aminopeptidase inhibitors in human solid tumor cell lines. Int J Cancer. 2001, 94(4), 485-491.
[31] Sekine, K.; Fujii, H. Abe, F. Induction of apoptosis by bestatin (ubenimex) in human leukemic cell lines. Leukemia. 1999, 13(5), 729-734.
[32] Lin, M.; He, J.; Cai, Z. Qian, W. [Aminopeptidase inhibitor Bestatin induces HL-60 cell apoptosis through activating caspase 3]. Zhonghua Xue Ye Xue Za Zhi. 2001, 22(7), 348-350.
[33] Kanthou, C.; Greco, O.; Stratford, A.; Cook, I.; Knight, R.; Benzakour, O. Tozer, G. The tubulin-binding agent combretastatin A-4-phosphate arrests endothelial cells in mitosis and induces mitotic cell death. Am J Pathol. 2004, 165(4), 1401-1411.
[34] Iyer, S.; Chaplin, D. J.; Rosenthal, D. S.; Boulares, A. H.; Li, L. Y. Smulson, M. E. Induction of apoptosis in proliferating human endothelial cells by the tumor-specific antiangiogenesis agent combretastatin A-4. Cancer Res. 1998, 58(20), 4510-4514.
[35] Woods, C. M.; Zhu, J.; McQueney, P. A.; Bollag, D. Lazarides, E. Taxol-induced mitotic block triggers rapid onset of a p53-independent apoptotic pathway. Mol. Med. 1995, 1/(5), 506-526.
[36] Jordan, M. A.; Wendell, K.; Gardiner, S.; Derry, W. B.; Copp, H. Wilson, L. Mitotic block induced in HeLa cells by low concentrations of paclitaxel (Taxol) results in abnormal mitotic exit and apoptotic cell death. Cancer Res. 1996, 56(4), 816-825.
[37] Nicoletti, I.; Migliorati, G.; Pagliacci, M. C.; Grignani, F. Riccardi, C. A rapid and simple method for measuring thymocyte apoptosis by propidium iodide staining and flow cytometry. J Immunol Methods. 1991, 139(2), 271-279.
[38] Riccardi, C. and Nicoletti, I. Analysis of apoptosis by propidium iodide staining and flow cytometry. Nat. Protoc. 2006, 1(3), 1458-1461.
[40] Lin, C. F.; Chen, C. L.; Chiang, C. W.; Jan, M. S.; Huang, W. C. Lin, Y. S. GSK-3beta acts downstream of PP2A and the PI 3-kinase-Akt pathway, and upstream of caspase-2 in ceramide-induced mitochondrial apoptosis. J Cell Sci. 2007, 120(Pt 16), 2935-2943.
[41] Chiu, W. H.; Luo, S. J.; Chen, C. L.; Cheng, J. H.; Hsieh, C. Y.; Wang, C. Y.; Huang, W. C.; Su, W. C. Lin, C. F. Vinca alkaloids cause aberrant ROS-mediated JNK activation, Mc1-1 downregulation, DNA damage, mitochondrial dysfunction, and apoptosis in lung adenocarcinoma cells. Biochem Pharmacol. 2012, 83(9), 1159-1171.
[42] Sanchez, A. M.; Sanchez, M. G.; Malagarie-Cazenave, S.; Olea, N. Diaz-Laviada, I. Induction of apoptosis in prostate tumor PC-3 cells and inhibition of xenograft prostate tumor growth by the vanilloid capsaicin. Apoptosis. 2006, 11(1), 89-99.

1-43. (canceled)

44. A compound of general formula IIA or IIIA 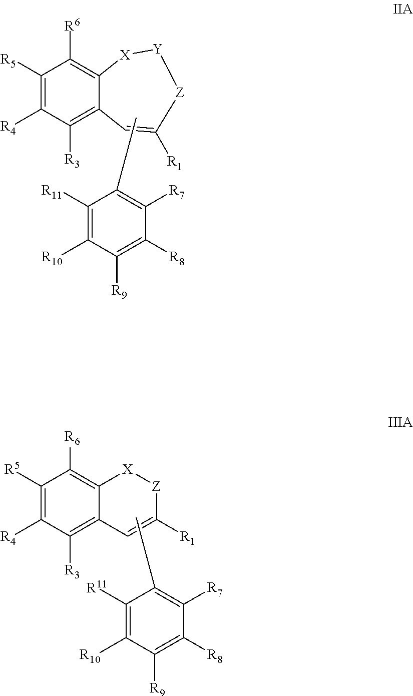
or a pharmaceutically acceptable salt thereof, wherein
X, Y and Z is each, independently, selected from the group consisting of a heteroatom, CH, CH₂, S═O, C═O, C═S, C(H)R, C(R)₂, N(R), C═NR, C═C(R)₂, C(L)W, C(H)LW, C(LW)₂, N(LW), C═N(LW), C═C(LW)₂, with the proviso that at least one of X, Y or Z is C═O, C═S, C═NR, C═C(R)₂, C(H)LW, C═N(LW), C═CH(LW)— L is absent or any linker typically selected from O, S or oxidised forms thereof, NH, CH₂, O-alkyl, CH₂O, CH₂NH, and CH₂NHCOCH₂;
W is a binding agent for a target that is preferentially expressed on vasculature undergoing angiogenesis, and not expressed on quiescent vasculature, or an anti-angiogenic agent;
R, R₁ and R₃ are each, independently, any substituent, typically selected from the groups consisting of H, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkoxycarbonylaminohydroxyl, aminocarbonyl, alkylthiocarbonyl, alkoxy, phosphate, phosphonato, phosphinato, cyano, amino including alkyl amino, dialkylamino, arylamino, diarylamino, and alkylarylamino, acylamino including alkylcarbonylamino, arylcarbonylamino, carbamoyl, and ureido, amidino, imino, sulfhydryl, alkylthio, arylthio, thiocarboxylate, sulphate, sulfonato, sulfamoyl, sulfonamido, nitro including nitrile, trifluoromethyl, isocyanate, isothiocyanate, azido, heterocycle, or a substituted or unsubstituted aromatic or heterocyclic ring structure in which the substituents (if included) are each, independently, selected from the groups consisting of W, (L)W, H, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkoxycarbonylaminohydroxyl aminocarbonyl, alkylthiocarbonyl, alkoxy, phosphate, phosphonato, phosphinato, cyano, amino including alkyl amino, dialkylamino, arylamino, diarylamino, and alkylarylamino, acylamino including alkylcarbonylamino, arylcarbonylamino, carbamoyl, and ureido, amidino, imino, sulfhydryl, alkylthio, arylthio, thiocarboxylate, sulphate, sulfonato, sulfamoyl, sulfonamido, nitro including nitrile, trifluoromethyl, isocyanate, isothiocyanate, azido, heterocycle;
R₄, R₅, and R₆ are each, independently, lower alkoxy substituents; and
R₇ to R₁₁ are each, independently, selected from the groups consisting of H, halogen, hydroxyl or derivatives thereof, amino or derivatives thereof alkoxy, phosphate, amidino, sulfhydryl or derivatives thereof, alkylthio and L(W).

45. A compound of claim 44 in which at least one of X, Y and Z is C═O.

46. A compound of claim 44 in which R₁ and R₃ are each independently selected from H and a halogen.

47. A compound of claim 44 in which R₈ and R₁₀ are each, independently, selected from H, OH, amino and L(W).

48. A compound according to claim 44 in which R₇ and R₁₁ are each, independently, selected from H, NH₂, lower alkoxy, alkylthio and OH.

49. A compound according to claim 44 in which R₉ is a lower alkoxy group in the para position.

50. A compound according to claim 44 in which R₁ and R₃ are each independently selected from H and a halogen, R₈ and R₁₀ are each, independently, selected from OH, amino and L(W), and R₇ and R₁₁ are each, independently, selected from H and OH.

51. A compound as claimed in claim 44 in which X is a heteroatom or CH₂.

52. A compound of claim 47 in which X is O, and at least one of Y or Z is C═O, C═S, C═NR, C═C(R)₂, C(H)LW, C═N(LW), C═CH(LW).

53. A compound as claimed in claim 44 in which W is selected from an APA substrate, an APA inhibitor, an APN substrate, an APN inhibitor, an alkaline phosphatase substrate, or an anti-angiogenic drug.

54. A compound as claimed in claim 44 in which at least one of X, Y and Z is selected from C(H)LW, C═N(LW), C═CH(LW), and in which W is selected from an APA or APN inhibitor.

55. A compound as claimed in claim 44 in which at least one of R₈ and R₁₀ is (L)W, in which L is absent or is O or NH and W is selected from an APA or APN inhibitor. [symbol] (L)W, in which L is absent or is O or NH and W is selected from an APA, APN or phosphatase substrate.

57. A compound as claimed in claim 44 in which at least one of R₇ to R₁₁ is a lower alkoxy group and at least one of R₇ to R₁₁ is selected from OH, NH₂, W or L(W).

58. A compound as claimed in claim 44 in which R₉ is a lower alkoxy group and R₈/R₁₀ is selected from H, OH, NH₂, W and L(W).
